SEQUENCE LISTING (226 sequences)

SEQ ID NO: 1 - DNA, 1491 bp, Artificial Sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtattagctctttctttccatgggttggcctcgccgcgtgggctgagggaaggactgtcctgggactggacaggcgggttatgggacctgaaaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 2 - DNA, 1302 bp, Artificial Sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgcggaaaaccgcgggataccgcgatgattgcgagctagtcgacaatcaagagggagaaaaggggattgtcgagagaggaaagaaggagctcaaatgcaggagagagggagaaagcatttgagaagaagggagagaggaacaactcgtggaggagagaaaagagacgagttgtgaagaagagagaggaggctttagctcgcttcgttttcatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaaggacctttgagaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagt

SEQ ID NO: 3 - DNA, 404 bp, Artificial Sequence
  Synthetic nucleic acid sequence
gtaagtattagctctttctttccatgggttggcctcgccgcgtgggctgagggaaggactgtcctgggactggacaggcgggttatgggacctgaaaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtacc

SEQ ID NO: 4 - DNA, 382 bp, Artificial Sequence
  Synthetic nucleic acid sequence
acttctaggcgcgccgcggaaaaccgcgggataccgcgatgattgcgagctagtcgacaatcaagagggagaaaaggggattgtcgagagaggaaagaaggagctcaaatgcaggagagagggagaaagcatttgagaagaagggagagaggaacaactcgtggaggagagaaaagagacgagttgtgaagaagagagaggaggctttagctcgcttcgttttcatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccag

SEQ ID NO: 5 - DNA, 489 bp, Artificial Sequence
  Synthetic nucleic acid sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaag

SEQ ID NO: 6 - DNA, 237 bp, Artificial Sequence
  Synthetic nucleic acid sequence
gtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaaggacctttga

SEQ ID NO: 7 - DNA, 382 bp, Artificial Sequence
  Synthetic nucleic acid sequence
acttctaggcgcgccgcggaaaaccgcgggataccgcgatgattgcgagctagggagagagaggggaaagaaaagagaaagaggaggaggaaagaggggagagaggggagggaaaggagagaagggaggaagggaagaaagaaagaagaggaaaagaggggaggaggaggagaaaggagaaaaaaagaagggaagggagaaaggctttagctcgcttcgttttcatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccag

SEQ ID NO: 8 - DNA, 301 bp, Artificial Sequence
  Synthetic nucleic acid sequence
gtaagtgtcccgcggaacattattataacgttgctcgaagatatcagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgactctaga

SEQ ID NO: 9 - DNA, 326 bp, Artificial Sequence
  Synthetic nucleic acid sequence
gctagcgtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatctccgcggaacattattataacgttgctcgaatactaactggtacctcttcttttttttttgatatctgcag

SEQ ID NO: 10 - DNA, 278 bp, Artificial Sequence
  Synthetic nucleic acid sequence
gttgcctttacttctggcgcgccaaaaggcgtgccagaagtaccgggctaataatgtttcgcggtcctcttaaatctgcctaaatacgtataaatttgatcgccctgaaaaagggcgatcaaagccctgaaaaagggcatacgtagccctgaaaaagggcaggcagagccctgaaaaagggcaagaggaccgcggaacattattagccgccaccatggacaggcgggttatgggacctgaaaatactaacaatcgattttttttccctttttttccag

SEQ ID NO: 11 - DNA, 190 bp, Artificial Sequence
  Synthetic nucleic acid sequence
acttctaggcgcgccgcggaaaaccgcgggatatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccag

SEQ ID NO: 12 - DNA, 459 bp, Artificial Sequence
  Synthetic nucleic acid sequence
gtaagtattagctctttctttccatgggttggcctcgccgcgtgggctgagggaaggactgtcctgggactggacaggcgggttatgggacctgaaaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 13 - DNA, 382 bp, Artificial Sequence
  Synthetic nucleic acid sequence
acttctaggcgcgccgcggaaaaccgcgggataccgcgatgattgcgagctagtcgacaatcaagagggagaaaaggggattgtcgagagaggaaagaaggagctcaaatgcaggagagagggagaaagcatttgagaagaagggagagaggaagaactcctggaggagagaaaagagaggagttctgaagaagagagaggaggctttagctcgcttcgttttcatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccag

SEQ ID NO: 14 - DNA, 372 bp, Artificial Sequence
  Synthetic nucleic acid sequence
gtaagtattaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 15 - DNA, 407 bp, Artificial Sequence
  Synthetic nucleic acid sequence
gtaagtattagctctttctttccatgggttggcctcgccgcgtgaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 16 - DNA, 378 bp, Artificial Sequence
  Synthetic nucleic acid sequence
gtaagtattagctctttctttccatgggttggcctcgccgcgtgggctgagggaaggactgtcctgggactggacaggcgggttatgggacctgaaaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcggtaccaataaaatatctttattttcattacatctgtgtgttggttttttgtgtgactagt

SEQ ID NO: 17 - DNA, 309 bp, Artificial Sequence
  Synthetic nucleic acid sequence
acttctaggcgcgccgcggaaaaccgcgggataccgcgatgattgcgagctagtcgacaatcaagagggagaaaaggggattgtcgagagaggaaagaaggagctcaaatgcaggagagagggagaaagcatttgagaagaagggagagaggaagaactcctggaggagagaaaagagaggagttctgaagaagagagaggaggctttagctcgcttcgttttcatcattattgcggccctgaaaaagggccgcttataacgttgctcgaattctactaacaatcgattttttttccctttttttccag

SEQ ID NO: 18 - DNA, 419 bp, Artificial Sequence
  Synthetic nucleic acid sequence
atatccttttagggcagagtgaagagttaggaggaaggtggttgggagagggatttccaggccttaggacatcatgacagatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 19 - DNA, 275 bp, Artificial Sequence
  Synthetic nucleic acid sequence
acttctaggcgcgccgcggaaaaccgcgggataccgcgatgattgcgagctagtcgacaatcaagagggagaaaaggggattgtcgagagaggaaagaaggagctcaaatgcaggagagagggagaaagcatttgagaagaagggagagaggaacaactcgtggaggagagaaaagagacgagttgtgaagaagagagaggaggctttagctcgcttcgttttcatcatttccaggccttaggacatcatgacatttttccttaactttgctcac

SEQ ID NO: 20 - DNA, 3975 bp, Artificial Sequence
  Synthetic nucleic acid sequence
acttctaggcgcgccgccaccatgggattcgtgcggcagattcagctgctgctgtggaagaactggaccctgcggaagcggcagaaaatcagattcgtggtggaactcgtgtggcccctgagcctgtttctggtgctgatctggctgcggaacgccaatcctctgtacagccaccacgagtgtcacttccccaacaaggccatgccttctgccggaatgctgccttggctgcagggcatcttctgcaacgtgaacaacccctgctttcaaagccccacacctggcgaaagccctggcatcgtgtccaactacaacaacagcatcctggccagagtgtaccgggacttccaagagctgctgatgaacgcccctgagtctcagcacctgggcagaatctggaccgagctgcacatcctgagccagttcatggacaccctgagaacacaccccgagagaatcgccggcaggggcatcagaatccgggacatcctgaaggacgaggaaaccctgacactgttcctcatcaagaacatcggcctgagcgacagcgtggtgtacctgctgatcaacagccaagtgcggcccgagcagtttgctcatggcgtgccagatctcgccctgaaggatatcgcctgttctgaggccctgctggaacggttcatcatcttcagccagcggagaggcgccaagaccgtcagatatgccctgtgcagtctgagccagggaaccctgcagtggatcgaggataccctgtacgccaacgtggacttcttcaagctgttccgggtgctgcccacactgctggattctcggtcccaaggcatcaacctgagaagctggggcggcatcctgtccgacatgagcccaagaatccaagagttcatccaccggcctagcatgcaggacctgctgtgggttaccagacctctgatgcagaacggcggacccgagacattcaccaagctgatgggcattctgagcgatctgctgtgcggctaccctgaaggcggaggatctagagtgctgagcttcaattggtacgaggacaacaactacaaggccttcctgggcatcgactccaccagaaaggaccccatctacagctacgaccggcggacaaccagcttctgcaatgccctgatccagagcctggaaagcaaccctctgaccaagatcgcttggagggccgccaaacctctgctgatgggaaagatcctgtacacccctgacagccctgccgccagaagaatcctgaagaacgccaacagcaccttcgaggaactggaacacgtgcgcaagctggtcaaggcctgggaagaagtgggacctcagatctggtacttcttcgacaatagcacccagatgaacatgatcagagacaccctgggcaaccctaccgtgaaggacttcctgaacagacagctgggcgaagagggcattaccgccgaggccatcctgaactttctgtacaagggccccagagagtcccaggccgacgacatggccaacttcgattggcgggacatcttcaacatcaccgacagaaccctgcggctggtcaaccagtacctggaatgcctggtgctggacaagttcgagagctacaacgacgagacacagctgacccagagagccctgtctctgctggaagagaatatgttctgggctggcgtggtgttccccgacatgtacccttggacaagcagcctgcctcctcacgtgaagtacaagatccggatggacatcgacgtggtcgaaaagaccaacaagatcaaggaccggtactgggacagcggccctagagctgatcccgtggaagattttcgctacatctggggcggattcgcatacctgcaggacatggtggaacagggaatcacacggtcccaggtgcaggctgaagctcctgtgggaatctacctgcagcagatgccttatccttgcttcgtggacgacagcttcatgatcatcctgaatcggtgcttccccatcttcatggtgctggcctggatctactccgtgtctatgaccgtgaagtccatcgtgctggaaaaagagctgcggctgaaagagacactgaagaaccagggcgtgtccaatgccgtgatctggtgcacctggtttctggacagcttctccattatgagcatgagcatctttctgctgacgatcttcatcatgcacggccggatcctgcactacagcgacccctttatcctcttcctgttcctgctggccttctccaccgctacaatcatgctgtgttttctgctgtccaccttcttctccaaagcctctctggccgctgcttgtagcggcgtgatctacttcaccctgtacctgcctcacatcctgtgcttcgcatggcaggacagaatgaccgccgagctgaagaaagctgtgtccctgctgagccctgtggcctttggctttggcaccgagtacctcgtcagatttgaggaacaaggactgggactgcagtggtccaacatcggcaatagccctacagagggcgacgagttcagcttcctgctgtctatgcaaatgatgctgctggacgccgccgtgtatggactgctggcttggtatctggaccaggtgttccctgccgattacggcactcctctgccttggtatttcctgctgcaagagagctactggctcggcggcgagggatgtagcaccagagaagaaagagccctggaaaagaccgagcctctgaccgaggaaacagaggaccctgaacacccagagggcatccacgatagctttttcgagagagaacaccccggctgggtgccaggcgtgtgtgtgaagaatctggtcaagatcttcgagccctgcggcagacctgccgtggacagactgaacatcaccttctacgagaaccagattaccgcctttctgggccacaacggcgctggcaagacaaccacactgagcatcctcaccggcctgctgcctccaacaagcggcacagttctcgttggcggcagagacatcgagacaagcctggatgccgtcagacagtccctgggcatgtgccctcagcacaacatcctgtttcaccacctgaccgtggccgagcacatgctgttttatgcccagctgaagggcaagagccaagaagaggctcagctggaaatggaagccatgctcgaggacaccggcctgcaccacaagagaaatgaggaagcccaggatctgagcggcggcatgcagagaaaactgagcgtggccattgccttcgtgggcgacgccaaggttgtgatcctggatgagcctacaagcggcgtggacccttacagcagaagatccatctgggatctgctgctgaagtacagaagcggccggaccatcatcatgagcacccaccacatggacgaggccgatctgctcggagacagaatcgccatcattgctcagggcagactgtactgcagcggcaccccactgtttctgaagaactgtttcggcaccggactgtatctgaccctcgtgcggaagatgaagaacatccagtctcagcggaagggcagcgagggcacctgtagctgttctagcaagggctttagcaccacctgtccagctcacgtggacgatctgacccctgaacaggtgctggatggcgacgtgaacgagctgatggacgtggtgctgcaccatgtgcctgaggccaagctggtggaatgcatcggccaggtaagtattagctctttctttccatgggttggcctcgccgcgtgggctgagggaaggactgtcctgggactggacaggcgggttatgggacctgaagcgataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtgactagt

SEQ ID NO: 21 - DNA, 3611 bp, Artificial Sequence
  Synthetic nucleic acid sequence
aggatttttgacctgctcgattgtccactgcgagcaggtcttttggagtcgggcgaggcggaagcccgactccttttggcatgcacgctagccgcgtcgtgcatgccttttatcgaattcgggttatgggaccagtgaaggctgagggaaggactgtcctgggactggacaggcgggttatgggacctgaaaatactaacaatcgattttttttccctttttttccaggaactgatttttctgctcccgaacaagaacttcaagcaccgggcctacgccagcctgttcagagagctggaagaaaccctggccgacctgggcctgtctagctttggcatcagcgacacccctctcgaagagatcttcctgaaagtgacagaggacagcgatagcggccctctgtttgctggcggagcacagcaaaagcgcgagaacgtgaaccctagacacccctgtctgggcccaagagagaaagccggacagacccctcaggacagcaatgtgtgctctcctggtgctcctgccgctcatcctgagggacaacctccacctgaacctgagtgtcctggacctcagctgaacaccggaacacagctggttctgcagcacgtgcaggctctgctcgtgaagagattccagcacaccatcagaagccacaaggactttctggcccagatcgtgctgcccgccacctttgtttttctggctctgatgctgagcatcgtgatccctccattcggcgagtaccccgctctgacactgcacccttggatctacggccagcagtacacctttttctccatggacgaacccggcagcgagcagttcacagtgctggctgatgtcctgctgaacaagcccggcttcggcaaccggtgtctgaaagaaggatggctgcctgagtacccttgcggcaacagcacaccttggaaaacccctagcgtgtcccctaacatcacccagctgttccaaaagcagaaatggacccaagtgaacccctctccatcctgccggtgctccacaagggaaaagctgaccatgctgcccgagtgtccagaaggcgctggcggacttcctccacctcagagaacacagagatccaccgagattctccaggacctgaccgaccggaatatcagcgacttcctggttaagacataccccgcactgatccggtccagcctgaagtccaagttctgggtcaacgaacagagatacggcggcatcagcatcggcggaaaactgcctgtggtgcctatcacaggcgaggcccttgtgggctttctgtccgatctggggagaatcatgaacgtgtccggcggacctatcaccagggaagccagcaaagagatccccgatttcctgaagcacctggaaaccgaggacaatatcaaagtgtggttcaacaacaaaggatggcacgccctcgtgtcttttctgaacgtggcccacaatgccatcctgcgggctagcctgcctaaggacagaagccctgaggaatacggcatcaccgtgatctcccagcctctgaatctgaccaaagagcagctgagcgagatcaccgtgctgaccacctctgtggatgctgtggtggccatctgcgtgatcttcagcatgagcttcgtgcccgcctccttcgtgctgtacctgattcaagagagagtgaacaagagcaagcacctccagttcatctccggggtgtccccaaccacctactgggtcaccaattttctgtgggacatcatgaactacagcgtgtcagccggcctggtcgtgggcatctttatcggctttcaaaagaaggcctacacgagccccgagaacctgcctgctttggttgctctgctgctcctgtatggctgggccgtgattcccatgatgtaccccgccagctttctgtttgacgtgcccagcacagcctacgtggccctgtcttgcgccaatctgttcatcggcatcaacagcagcgccatcacattcatcctggaactgttcgagaacaacaggaccctgctgcggttcaacgccgtgctgcggaaactgctgatcgtgttccctcacttctgtctcggccggggcctgatcgacctggctctgtctcaagccgtgaccgatgtgtacgccagatttggcgaggaacactccgccaatccattccactgggacctgatcggcaagaacctgttcgccatggtggtggaaggcgtcgtgtacttcctgctcactctgctggtgcagagacacttttttctgtcccaatggatcgccgagcctaccaaagaacccattgtggacgaggacgacgatgtggccgaggaaagacagagaatcatcaccggcggcaacaagaccgatatcctgagactgcacgagctgacaaagatctaccccggcacaagctccccagccgtggataggctttgtgtgggagttagacccggcgagtgctttggcctgctgggagttaatggcgccggaaagaccaccaccttcaagatgctgaccggcgacaccacagtgacaagcggagatgctacagtggccggcaagagcatcctgaccaacatcagcgaagtgcatcagaacatgggctactgccctcagttcgacgccatcgacgaactgctgacaggccgcgaacacctgtatctgtatgccagactgagaggcgtgcccgctgaagagatcgagaaggtggccaactggtccatcaagtctctgggcctgacagtgtacgccgactgtctggccggaacatacagcggaggaaacaagcggaagctgagcaccgccattgctctgatcggatgcccacctctggtcctgctggatgaacccaccaccggaatggatccccaggctagaagaatgctctggaacgtgatcgtgtctatcatccgcgagggcagagctgtggtgctgacctctcactccatggaagagtgcgaggctctgtgtacccggctggccattatggtcaagggcgccttcagatgcatgggcaccattcagcatctgaaaagcaagttcggcgacggctacatcgtgacaatgaagatcaagagccccaaggacgacctcctgcctgatctgaaccccgtggaacagttttttcagggcaacttccccggctccgtgcagcgggaaagacactataacatgctgcagtttcaggtgtcctcctccagcctggctcggatctttcaactgctgctctctcacaaggacagcctgctgattgaagagtacagcgtgacacagaccacactcgaccaggttttcgtgaacttcgccaagcagcagaccgagagccacgacctgcctctgcatcctcgggccgctggtgcctctagacaagctcaggacggcgctcgggctgactacaaagaccatgacggtgattataaagatcatgacatcgactataaggatgacgatgacaaatgaggtaccaattcctcacctgcgatctcgagctttatttgtgaaatttgtgatgctattgctttatttgtaaccattataagctgcaataaacaagttaacaacaacaattgcattcattttatgtttcaggttcagggggaggtgtgggaggttttttaaactag

SEQ ID NO: 22 - DNA, 3975 bp, Artificial Sequence
  Synthetic nucleic acid sequence
acttctaggcgcgccgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgccggaagcggagctactaacttcagcctgctgaagcaggctggagacgtggaggagaaccctggacctaggcgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtattagctctttctttccatgggttggcctcgccgcgtgggctgagggaaggactgtcctgggactggacaggcgggttatgggacctgaagcgataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtgactagt

SEQ ID NO: 23 - DNA, 3912 bp, Artificial Sequence
  Synthetic nucleic acid sequence
aggatttttgacctgctcgattgtccactgcgagcaggtcttttggagtcgggcgaggcggaagcccgactccttttggcatgcacgctagccgcgtcgtgcatgccttttatcttcgggttatgggaccagtgaaggctgagggaaggactgtcctgggactggacaggcgggttatgggacctgaaaatactaacaatcgattttttttccctttttttccaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaaggaccttggaagcggagctactaacttcagcctgctgaagcaggctggagacgtggaggagaaccctggacctatcacaaagaagcacacagcccacttctccaagaagggcgaagaggaaaacctggaaggcctgggcaatcagaccaagcagatcgtcgagaagtacgcctgcaccaccagaatcagccccaacacaagccagcagaacttcgtgacccagcggagcaaaagagccctgaagcagtttcggctgcccctggaagaaaccgagctggaaaagcggatcatcgtggacgacaccagcacacagtggtccaagaacatgaagcacttgacccctagcacactgacccagatcgactacaacgagaaagagaagggcgctatcacacagagcccactgagcgactgtctgaccagaagccacagcatccctcaggccaacagatcccctctgccaatcgccaaagtgtctagcttccccagcatcagacccatctacctgaccagagtgctgttccaggacaacagcagccatctgccagccgccagctaccggaagaaagatagcggcgtgcaagagtccagccactttctgcaaggcgctaagaagaacaatctgagcctggctattctgaccctggaaatgaccggcgatcagagagaagtcggctctctgggcaccagcgccacaaatagcgtgacctacaaaaaggtggaaaacaccgtgctgcctaagcctgacctgccaaagacaagcggcaaggtggaactgctgccaaaggtgcacatctaccagaaggacctgtttcctaccgagacaagcaacggctctcccggccatctggatctggtggaaggatctctgctgcagggaaccgagggcgccatcaagtggaacgaggccaatagacctggcaaggtgcccttcctgagagtggccacagagtctagcgccaagacaccctccaaactgctggatcccctggcctgggataaccactacggcactcagatccccaaagaggaatggaagtcccaagagaagtcccctgaaaagaccgccttcaagaagaaggacaccattctgtccctgaatgcctgcgagagcaaccacgccattgccgccatcaatgagggccagaacaagcccgagatcgaagtgacctgggccaagcagggaagaaccgagagactgtgctcccagaatcctcctgtgctgaagcggcaccagagagaaatcacccggaccacactgcagagcgaccaagaagagatcgattacgacgataccatcagcgtcgagatgaagaaagaagatttcgacatctacgacgaggacgagaatcagagccctcggagcttccagaagaaaaccaggcactactttattgccgccgtcgagcggctgtgggactacggaatgtctagctctcctcacgtgctgcggaatagagcccagtctggtagcgtgccccagttcaaaaaggtcgtgttccaagagttcaccgacggcagcttcacccagccactgtatagaggcgagctgaacgagcatctgggcctgctgggcccttatatcagagccgaagtggaagataacatcatggtcaccttccggaatcaggcctctcggccctacagcttctacagctccctgatctcctacgaagaggaccagagacagggcgcagagccccggaagaatttcgtgaagcccaacgagactaagacctacttttggaaggtgcagcaccatatggcccctacaaaggacgagttcgactgcaaagcctgggcctacttctccgatgtggacctcgagaaggatgtgcacagcggactcatcggcccactgcttgtgtgccacaccaacacactgaaccccgctcacggcagacaagtgacagtgcaagaattcgccctgtttttcaccatcttcgacgaaacgaagtcctggtacttcaccgaaaacatggaaagaaactgcagggccccttgcaacattcagatggaagatcccaccttcaaagagaactaccggttccacgccatcaacggctacatcatggacacactgcccggcctggttatggctcaggatcagagaatccggtggtatctgctgtccatgggctccaacgagaatatccactccatccacttctccggccacgtgttcaccgtgcggaaaaaagaagagtacaaaatggccctgtacaatctgtaccctggggtgttcgaaaccgttgagatgctgcctagcaaggccggaatttggagagtggaatgtctgattggagagcacctccacgccgggatgagcaccctgtttctggtgtactccaacaagtgtcagacccctctcggcatggcctctggccacattagagacttccagatcaccgccagcggacagtatggacagtgggcccctaaactggccagactgcactactccggcagcatcaatgcctggtccaccaaagagcctttcagctggatcaaagtggacctgctggctcccatgatcatccacggaatcaagacccagggcgccagacaaaagttcagcagcctgtacatcagccagttcatcatcatgtacagcctggacggaaagaagtggcagacctaccggggcaatagcaccggcacactgatggtgttcttcggcaacgtggactccagcggcattaagcacaacatcttcaaccctccaatcattgcccgatacatccggctgcaccccacacactacagcatcaggtctaccctgagaatggaactgatgggctgcgacctgaacagctgcagcatgcccctcggaatggaaagcaaggccatcagcgacgcccagatcacagcctctagctacttcaccaacatgttcgccacttggagcccctctaaggcccggcttcatctgcaaggcagaagcaacgcttggaggccccaagtgaacaaccccaaagaatggctgcaggtcgactttcagaaaaccatgaaagtgacaggcgtgaccacacagggcgtcaagtccctgctgacctctatgtacgtgaaagagtttctgatcagctccagccaggacggccaccagtggaccctgttcttccaaaacggcaaagtgaaagtgttccagggaaatcaggacagcttcacacccgtggtcaactccctggatcctccactgctgacaagatacctgcggattcaccctcagtcttgggtgcaccagattgccctgcggatggaagtgctgggctgtgaagctcaggacctctactgaggtaccaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactag

SEQ ID NO: 24 - DNA, 3828 bp, Artificial Sequence
  Synthetic nucleic acid sequence
acttctaggcgcgccgccaccatgtacccatacgatgttccagattacgcttatccttatgacgtgcctgactacgcctatccctacgacgtccccgactatgcagtgtacaagaaaaccctgttcgtggaattcaccgaccacctgttcaatatcgccaagcctcggcctccttggatgggactgctgggacctacaattcaggccgaggtgtacgacaccgtggtcatcaccctgaagaacatggccagccatcctgtgtctctgcacgccgtgggagtgtcttactggaaggcttctgagggcgccgagtacgacgatcagacaagccagagagagaaagaggacgacaaggttttccctggcggcagccacacctatgtctggcaagtcctgaaagaaaacggccctatggcctccgatcctctgtgcctgacatacagctacctgagccacgtggacctggtcaaggacctgaattctggcctgatcggagccctgctcgtgtgtagagaaggcagcctggccaaagagaaaacccagacactgcacaagttcatcctgctgttcgccgtgttcgacgagggcaagagctggcacagcgagacaaagaacagcctgatgcaggacagggatgccgcctctgctcgggcttggcctaagatgcacaccgtgaacggctacgtgaacagaagcctgcctggactgatcggctgccacagaaagtccgtgtactggcacgtgatcggcatgggcacaacacctgaggtgcacagcatctttctggaaggacacaccttcctcgtgcggaaccatagacaggccagcctggaaatcagccctatcaccttcctgaccgctcagaccctgctgatggatctgggccagtttctgctgttctgccacatcagctcccaccagcacgatggcatggaagcctacgtgaaggtggacagctgccccgaagaaccccagctgcggatgaagaacaacgaggaagccgaggactacgacgacgacctgaccgactctgagatggacgtcgtcagattcgacgacgataacagccccagcttcatccaaatcagaagcgtggccaagaagcaccccaagacctgggtgcactatatcgccgccgaggaagaggactgggattacgctcctctggtgctggcccctgacgacagaagctacaagagccagtacctgaacaacggccctcagcggatcggccggaagtataagaaagtgcggttcatggcctacaccgacgagacattcaagaccagagaggccatccagcacgagagcggaattctgggccctctgctgtatggcgaagtgggcgatacactgctgatcatcttcaagaaccaggccagcagaccctacaacatctaccctcacggcatcaccgatgtgcggcccctgtattctagaaggctgcccaagggcgtgaagcacctgaaggacttccctatcctgcctggcgagatcttcaagtacaagtggaccgtgaccgtggaagatggccccaccaagagcgaccctagatgtctgacacggtactacagcagcttcgtgaacatggaacgcgacctggccagcggcctgattggacctctgctgatctgctacaaagaaagcgtggaccagcggggcaaccagatcatgagcgacaagcggaacgtgatcctgtttagcgtgttcgatgagaaccggtcctggtatctgaccgagaacatccagcggtttctgcccaatcctgctggcgtgcagctggaagatcctgagttccaggcctccaacatcatgcactccatcaatggctatgtgttcgacagcctgcagctgagcgtgtgcctgcacgaagtggcctactggtacatcctgagcattggcgcccagaccgacttcctgtccgtgttcttttccggctacaccttcaagcacaagatggtgtacgaggataccctgacactgttcccattctccggcgagacagtgttcatgagcatggaaaaccccggcctgtggatcctgggctgtcacaacagcgacttccggaacagaggcatgacagccctgctgaaggtgtccagctgcgacaagaacaccggcgactactacgaggacagctatgaggacatcagcgcctacctgctgagcaagaacaatgccatcgagcccagaagcttcagccagaatagcagacacccctccaccagacagaagcagttcaacgccacaacaatccccgagaacgacatcgagaaaaccgatccttggtttgcccaccggacccctatgcctaagatccagaacgtgtcctccagcgatctgctgatgctcctgagacagagccctacacctcacggactgagcctgtccgatctgcaagaggccaaatacgaaaccttcagcgacgacccttctcctggcgccatcgacagcaacaatagcctgagcgagatgacccacttcagaccacagctgcaccacagcggcgacatggtgtttacacctgagagcggcctccagctgagactgaatgagaagctgggaaccaccgccgccaccgagctgaagaaactggacttcaaggtgtcctctaccagcaacaacctgatcagcacaatcccctccgacaacctggctgccggcaccgacaacacatcttctctgggcccacctagcatgcccgtgcactacgatagccagctggataccacactgttcggcaagaagtctagccctctgacagagtctggcggccctctgtctctgagcgaggaaaacaacgacagcaagctgctggaatccggcctgatgaacagccaagagtcctcctggggcaagaatgtgtccagcaccgagtccggcagactgttcaagggaaagagagcccacggacctgctctgctgaccaaggataacgccctgttcaaagtgtccatcagcctgctcaagaccaacaagacctccaacaactccgccaccaacagaaagacccacatcgacggccctagcctgctgatcgagaatagccctagcgtctggcagaatatcctggaaagcgacaccgagttcaagaaagtgacccctctgatccacgaccggatgctcatggacaagaacgccaccgctctgcggctgaaccacatgagcaacaagacaaccagcagcaagaatatggaaatggtgcagcagaagaaagagggccccattcctccagacgctcagaaccccgatatgagcttcttcaagatgctctttctgcccgagagcgcccggtggatccagagaacacacggcaagaactccctgaactccggccagggaccttctccaaagcagctggtttccctgggacctgagaagtccgtggaaggccagaacttcctgagcgaaaagaacaaagtggtcgtcggcaagggcgagttcaccaaggatgtgggcctgaaagagatggtctttcccagcagccggaacctgttcctgaccaacctggacaacctgcacgagaacaacacccacaatcaagagaagaagatccaagaggtaagtattagctctttctttccatgggttggcctcgccgcgtgggctgagggaaggactgtcctgggactggacaggcgggttatgggacctgaagcgataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtg

SEQ ID NO: 25 - DNA, 3802 bp, Artificial Sequence
  Synthetic nucleic acid sequence
aggatttttgacctgctcgattgtccactgcgagcaggtcttttggagtcgggcgaggcggaagcccgactccttttggcatgcacgctagccgcgtcgtgcatgccttttatcttcgggttatgggaccagtgaaggctgagggaaggactgtcctgggactggacaggcgggttatgggacctgaaaatactaacaatcgattttttttccctttttttccaggaaatcgaaaagaaagagacactcatccaagagaacgtggtgctgcctcagatccacacagtgaccggcaccaagaactttatgaagaatctgttcctgctgagtacccggcagaatgtggaaggcagctacgacggcgcttatgcccctgtgctgcaagacttcagatccctgaacgactccaccaatcggacaaagaagcacacagcccacttctccaagaagggcgaagaggaaaacctggaaggcctgggcaatcagaccaagcagatcgtcgagaagtacgcctgcaccaccagaatcagccccaacacaagccagcagaacttcgtgacccagcggagcaaaagagccctgaagcagtttcggctgcccctggaagaaaccgagctggaaaagcggatcatcgtggacgacaccagcacacagtggtccaagaacatgaagcacttgacccctagcacactgacccagatcgactacaacgagaaagagaagggcgctatcacacagagcccactgagcgactgtctgaccagaagccacagcatccctcaggccaacagatcccctctgccaatcgccaaagtgtctagcttccccagcatcagacccatctacctgaccagagtgctgttccaggacaacagcagccatctgccagccgccagctaccggaagaaagatagcggcgtgcaagagtccagccactttctgcaaggcgctaagaagaacaatctgagcctggctattctgaccctggaaatgaccggcgatcagagagaagtcggctctctgggcaccagcgccacaaatagcgtgacctacaaaaaggtggaaaacaccgtgctgcctaagcctgacctgccaaagacaagcggcaaggtggaactgctgccaaaggtgcacatctaccagaaggacctgtttcctaccgagacaagcaacggctctcccggccatctggatctggtggaaggatctctgctgcagggaaccgagggcgccatcaagtggaacgaggccaatagacctggcaaggtgcccttcctgagagtggccacagagtctagcgccaagacaccctccaaactgctggatcccctggcctgggataaccactacggcactcagatccccaaagaggaatggaagtcccaagagaagtcccctgaaaagaccgccttcaagaagaaggacaccattctgtccctgaatgcctgcgagagcaaccacgccattgccgccatcaatgagggccagaacaagcccgagatcgaagtgacctgggccaagcagggaagaaccgagagactgtgctcccagaatcctcctgtgctgaagcggcaccagagagaaatcacccggaccacactgcagagcgaccaagaagagatcgattacgacgataccatcagcgtcgagatgaagaaagaagatttcgacatctacgacgaggacgagaatcagagccctcggagcttccagaagaaaaccaggcactactttattgccgccgtcgagcggctgtgggactacggaatgtctagctctcctcacgtgctgcggaatagagcccagtctggtagcgtgccccagttcaaaaaggtcgtgttccaagagttcaccgacggcagcttcacccagccactgtatagaggcgagctgaacgagcatctgggcctgctgggcccttatatcagagccgaagtggaagataacatcatggtcaccttccggaatcaggcctctcggccctacagcttctacagctccctgatctcctacgaagaggaccagagacagggcgcagagccccggaagaatttcgtgaagcccaacgagactaagacctacttttggaaggtgcagcaccatatggcccctacaaaggacgagttcgactgcaaagcctgggcctacttctccgatgtggacctcgagaaggatgtgcacagcggactcatcggcccactgcttgtgtgccacaccaacacactgaaccccgctcacggcagacaagtgacagtgcaagaattcgccctgtttttcaccatcttcgacgaaacgaagtcctggtacttcaccgaaaacatggaaagaaactgcagggccccttgcaacattcagatggaagatcccaccttcaaagagaactaccggttccacgccatcaacggctacatcatggacacactgcccggcctggttatggctcaggatcagagaatccggtggtatctgctgtccatgggctccaacgagaatatccactccatccacttctccggccacgtgttcaccgtgcggaaaaaagaagagtacaaaatggccctgtacaatctgtaccctggggtgttcgaaaccgttgagatgctgcctagcaaggccggaatttggagagtggaatgtctgattggagagcacctccacgccgggatgagcaccctgtttctggtgtactccaacaagtgtcagacccctctcggcatggcctctggccacattagagacttccagatcaccgccagcggacagtatggacagtgggcccctaaactggccagactgcactactccggcagcatcaatgcctggtccaccaaagagcctttcagctggatcaaagtggacctgctggctcccatgatcatccacggaatcaagacccagggcgccagacaaaagttcagcagcctgtacatcagccagttcatcatcatgtacagcctggacggaaagaagtggcagacctaccggggcaatagcaccggcacactgatggtgttcttcggcaacgtggactccagcggcattaagcacaacatcttcaaccctccaatcattgcccgatacatccggctgcaccccacacactacagcatcaggtctaccctgagaatggaactgatgggctgcgacctgaacagctgcagcatgcccctcggaatggaaagcaaggccatcagcgacgcccagatcacagcctctagctacttcaccaacatgttcgccacttggagcccctctaaggcccggcttcatctgcaaggcagaagcaacgcttggaggccccaagtgaacaaccccaaagaatggctgcaggtcgactttcagaaaaccatgaaagtgacaggcgtgaccacacagggcgtcaagtccctgctgacctctatgtacgtgaaagagtttctgatcagctccagccaggacggccaccagtggaccctgttcttccaaaacggcaaagtgaaagtgttccagggaaatcaggacagcttcacacccgtggtcaactccctggatcctccactgctgacaagatacctgcggattcaccctcagtcttgggtgcaccagattgccctgcggatggaagtgctgggctgtgaagctcaggacctctactgaggtaccaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagt

SEQ ID NO: 26 - DNA, 10 bp, Artificial Sequence
  Synthetic nucleic acid sequence
tggggggagg

SEQ ID NO: 27 - DNA, 10 bp, Artificial Sequence
  Synthetic nucleic acid sequence
gtagtgaggg

SEQ ID NO: 28 - DNA, 10 bp, Artificial Sequence
  Synthetic nucleic acid sequence
gttggtggtt

SEQ ID NO: 29 - DNA, 10 bp, Artificial Sequence
  Synthetic nucleic acid sequence
agttgtggtt

SEQ ID NO: 30 - DNA, 10 bp, Artificial Sequence
  Synthetic nucleic acid sequence
gtattgggtc

SEQ ID NO: 31 - DNA, 10 bp, Artificial Sequence
  Synthetic nucleic acid sequence
agtgtgaggg

SEQ ID NO: 32 - DNA, 10 bp, Artificial Sequence
  Synthetic nucleic acid sequence
gggtaatggg

SEQ ID NO: 33 - DNA, 10 bp, Artificial Sequence
  Synthetic nucleic acid sequence
tcattggggt

SEQ ID NO: 34 - DNA, 10 bp, Artificial Sequence
  Synthetic nucleic acid sequence
ggtgggggtc

SEQ ID NO: 35 - DNA, 10 bp, Artificial Sequence
  Synthetic nucleic acid sequence
ggttttgttg

SEQ ID NO: 36 - DNA, 10 bp, Artificial Sequence
  Synthetic nucleic acid sequence
tatactcccg

SEQ ID NO: 37 - DNA, 10 bp, Artificial Sequence
  Synthetic nucleic acid sequence
gtattcgatc

SEQ ID NO: 38 - DNA, 10 bp, Artificial Sequence
  Synthetic nucleic acid sequence
gtagttccct

SEQ ID NO: 39 - DNA, 10 bp, Artificial Sequence
  Synthetic nucleic acid sequence
gttaatagta

SEQ ID NO: 40 - DNA, 10 bp, Artificial Sequence
  Synthetic nucleic acid sequence
tgctggttag

SEQ ID NO: 41 - DNA, 10 bp, Artificial Sequence
  Synthetic nucleic acid sequence
ataggtaacg

SEQ ID NO: 42 - DNA, 10 bp, Artificial Sequence
  Synthetic nucleic acid sequence
tctgaattgc

SEQ ID NO: 43 - DNA, 10 bp, Artificial Sequence
  Synthetic nucleic acid sequence
tctgggtttg

SEQ ID NO: 44 - DNA, 10 bp, Artificial Sequence
  Synthetic nucleic acid sequence
cattctcttt

SEQ ID NO: 45 - DNA, 10 bp, Artificial Sequence
  Synthetic nucleic acid sequence
gtattggtgt

SEQ ID NO: 46 - DNA, 10 bp, Artificial Sequence
  Synthetic nucleic acid sequence
tttagatttg

SEQ ID NO: 47 - DNA, 10 bp, Artificial Sequence
  Synthetic nucleic acid sequence
ataagtactg

SEQ ID NO: 48 - DNA, 10 bp, Artificial Sequence
  Synthetic nucleic acid sequence
tagtctatta

SEQ ID NO: 49 - DNA, 10 bp, Artificial Sequence
  Synthetic nucleic acid sequence
aggtattgca

SEQ ID NO: 50 - DNA, 10 bp, Artificial Sequence
  Synthetic nucleic acid sequence
gtagattacg

SEQ ID NO: 51 - DNA, 10 bp, Artificial Sequence
  Synthetic nucleic acid sequence
gggcgggtgc

SEQ ID NO: 52 - DNA, 10 bp, Artificial Sequence
  Synthetic nucleic acid sequence
cgtttacaat

SEQ ID NO: 53 - DNA, 11 bp, Artificial Sequence
  Synthetic nucleic acid sequence
gtacagggatg

SEQ ID NO: 54 - DNA, 10 bp, Artificial Sequence
  Synthetic nucleic acid sequence
aatcagggga

SEQ ID NO: 55 - DNA, 10 bp, Artificial Sequence
  Synthetic nucleic acid sequence
ggaggttttg

SEQ ID NO: 56 - DNA, 10 bp, Artificial Sequence
  Synthetic nucleic acid sequence
gtattccctg

SEQ ID NO: 57 - DNA, 10 bp, Artificial Sequence
  Synthetic nucleic acid sequence
tggtaagatc

SEQ ID NO: 58 - DNA, 10 bp, Artificial Sequence
  Synthetic nucleic acid sequence
gtagttaagt

SEQ ID NO: 59 - DNA, 10 bp, Artificial Sequence
  Synthetic nucleic acid sequence
gttggtttgg

SEQ ID NO: 60 - DNA, 10 bp, Artificial Sequence
  Synthetic nucleic acid sequence
gtatttactt

SEQ ID NO: 61 - DNA, 10 bp, Artificial Sequence
  Synthetic nucleic acid sequence
gtaacggggt

SEQ ID NO: 62 - DNA, 10 bp, Artificial Sequence
  Synthetic nucleic acid sequence
tttttttctg

SEQ ID NO: 63 - DNA, 10 bp, Artificial Sequence
  Synthetic nucleic acid sequence
ggggaaggga

SEQ ID NO: 64 - DNA, 10 bp, Artificial Sequence
  Synthetic nucleic acid sequence
ttaccccggt

SEQ ID NO: 65 - DNA, 10 bp, Artificial Sequence
  Synthetic nucleic acid sequence
gtattctatg

SEQ ID NO: 66 - DNA, 10 bp, Artificial Sequence
  Synthetic nucleic acid sequence
aggtattgtg

SEQ ID NO: 67 - DNA, 10 bp, Artificial Sequence
  Synthetic nucleic acid sequence
tttggggggg

SEQ ID NO: 68 - DNA, 10 bp, Artificial Sequence
  Synthetic nucleic acid sequence
gttgttagcg

SEQ ID NO: 69 - DNA, 10 bp, Artificial Sequence
  Synthetic nucleic acid sequence
ggtagttggg

SEQ ID NO: 70 - DNA, 10 bp, Artificial Sequence
  Synthetic nucleic acid sequence
ctaagtactg

SEQ ID NO: 71 - DNA, 10 bp, Artificial Sequence
  Synthetic nucleic acid sequence
aaccatcttc

SEQ ID NO: 72 - DNA, 10 bp, Artificial Sequence
  Synthetic nucleic acid sequence
gtacctgggt

SEQ ID NO: 73 - DNA, 10 bp, Artificial Sequence
  Synthetic nucleic acid sequence
gtatctcatt

SEQ ID NO: 74 - DNA, 10 bp, Artificial Sequence
  Synthetic nucleic acid sequence
aaataaaatt

SEQ ID NO: 75 - DNA, 10 bp, Artificial Sequence
  Synthetic nucleic acid sequence
ggtgggttat

SEQ ID NO: 76 - DNA, 10 bp, Artificial Sequence
  Synthetic nucleic acid sequence
taagggaggg

SEQ ID NO: 77 - DNA, 10 bp, Artificial Sequence
  Synthetic nucleic acid sequence
tatgggaggg

SEQ ID NO: 78 - DNA, 10 bp, Artificial Sequence
  Synthetic nucleic acid sequence
gatgggaggg

SEQ ID NO: 79 - DNA, 10 bp, Artificial Sequence
  Synthetic nucleic acid sequence
tggggggggt

SEQ ID NO: 80 - DNA, 10 bp, Artificial Sequence
  Synthetic nucleic acid sequence
ggggaagggg

SEQ ID NO: 81 - DNA, 10 bp, Artificial Sequence
  Synthetic nucleic acid sequence
tggtaagagg

SEQ ID NO: 82 - DNA, 10 bp, Artificial Sequence
  Synthetic nucleic acid sequence
gggttagggt

SEQ ID NO: 83 - DNA, 10 bp, Artificial Sequence
  Synthetic nucleic acid sequence
gtatcggggg

SEQ ID NO: 84 - DNA, 10 bp, Artificial Sequence
  Synthetic nucleic acid sequence
ggttttgctg

SEQ ID NO: 85 - DNA, 10 bp, Artificial Sequence
  Synthetic nucleic acid sequence
tgggggtgga

SEQ ID NO: 86 - DNA, 10 bp, Artificial Sequence
  Synthetic nucleic acid sequence
acttttagag

SEQ ID NO: 87 - DNA, 10 bp, Artificial Sequence
  Synthetic nucleic acid sequence
gtaacgggtt

SEQ ID NO: 88 - DNA, 10 bp, Artificial Sequence
  Synthetic nucleic acid sequence
gtttggggga

SEQ ID NO: 89 - DNA, 10 bp, Artificial Sequence
  Synthetic nucleic acid sequence
atttttagag

SEQ ID NO: 90 - DNA, 10 bp, Artificial Sequence
  Synthetic nucleic acid sequence
ttaaagtagg

SEQ ID NO: 91 - DNA, 10 bp, Artificial Sequence
  Synthetic nucleic acid sequence
gtattaatat

SEQ ID NO: 92 - DNA, 10 bp, Artificial Sequence
  Synthetic nucleic acid sequence
ggtttgggtg

SEQ ID NO: 93 - DNA, 10 bp, Artificial Sequence
  Synthetic nucleic acid sequence
tatgggaaag

SEQ ID NO: 94 - DNA, 10 bp, Artificial Sequence
  Synthetic nucleic acid sequence
ggttgggagg

SEQ ID NO: 95 - DNA, 10 bp, Artificial Sequence
  Synthetic nucleic acid sequence
gtatttagtg

SEQ ID NO: 96 - DNA, 10 bp, Artificial Sequence
  Synthetic nucleic acid sequence
gagttaaatg

SEQ ID NO: 97 - DNA, 10 bp, Artificial Sequence
  Synthetic nucleic acid sequence
ttgtaagttg

SEQ ID NO: 98 - DNA, 10 bp, Artificial Sequence
  Synthetic nucleic acid sequence
tgggggtagg

SEQ ID NO: 99 - DNA, 10 bp, Artificial Sequence
  Synthetic nucleic acid sequence
gttcttaggg

SEQ ID NO: 100 - DNA, 10 bp, Artificial Sequence
  Synthetic nucleic acid sequence
gtattctaag

SEQ ID NO: 101 - DNA, 10 bp, Artificial Sequence
  Synthetic nucleic acid sequence
ggaggttttg

SEQ ID NO: 102 - DNA, 10 bp, Artificial Sequence
  Synthetic nucleic acid sequence
agaatatgta

SEQ ID NO: 103 - DNA, 10 bp, Artificial Sequence
  Synthetic nucleic acid sequence
atctttcggg

SEQ ID NO: 104 - DNA, 10 bp, Artificial Sequence
  Synthetic nucleic acid sequence
ttgcattgaa

SEQ ID NO: 105 - DNA, 10 bp, Artificial Sequence
  Synthetic nucleic acid sequence
ggtgggattt

SEQ ID NO: 106 - DNA, 10 bp, Artificial Sequence
  Synthetic nucleic acid sequence
tttatctaat

SEQ ID NO: 107 - DNA, 10 bp, Artificial Sequence
  Synthetic nucleic acid sequence
gcgggtggtg

SEQ ID NO: 108 - DNA, 10 bp, Artificial Sequence
  Synthetic nucleic acid sequence
ggtttagata

SEQ ID NO: 109 - DNA, 10 bp, Artificial Sequence
  Synthetic nucleic acid sequence
tttatgcgtt

SEQ ID NO: 110 - DNA, 10 bp, Artificial Sequence
  Synthetic nucleic acid sequence
tgggtaaggc

SEQ ID NO: 111 - DNA, 10 bp, Artificial Sequence
  Synthetic nucleic acid sequence
gggggtggtc

SEQ ID NO: 112 - DNA, 10 bp, Artificial Sequence
  Synthetic nucleic acid sequence
gtagtatatt

SEQ ID NO: 113 - DNA, 10 bp, Artificial Sequence
  Synthetic nucleic acid sequence
ggaggtattt

SEQ ID NO: 114 - DNA, 10 bp, Artificial Sequence
  Synthetic nucleic acid sequence
gtattgtaag

SEQ ID NO: 115 - DNA, 10 bp, Artificial Sequence
  Synthetic nucleic acid sequence
tttacgggag

SEQ ID NO: 116 - DNA, 10 bp, Artificial Sequence
  Synthetic nucleic acid sequence
tagttctggg

SEQ ID NO: 117 - DNA, 10 bp, Artificial Sequence
  Synthetic nucleic acid sequence
ccacgtctat

SEQ ID NO: 118 - DNA, 10 bp, Artificial Sequence
  Synthetic nucleic acid sequence
agtgggtagg

SEQ ID NO: 119 - DNA, 10 bp, Artificial Sequence
  Synthetic nucleic acid sequence
caatttttac

SEQ ID NO: 120 - DNA, 10 bp, Artificial Sequence
  Synthetic nucleic acid sequence
ggtctggggg

SEQ ID NO: 121 - DNA, 10 bp, Artificial Sequence
  Synthetic nucleic acid sequence
atcaagattg

SEQ ID NO: 122 - DNA, 10 bp, Artificial Sequence
  Synthetic nucleic acid sequence
gttagctaaa

SEQ ID NO: 123 - DNA, 10 bp, Artificial Sequence
  Synthetic nucleic acid sequence
agtgtggggt

SEQ ID NO: 124 - DNA, 10 bp, Artificial Sequence
  Synthetic nucleic acid sequence
ggtatgtggg

SEQ ID NO: 125 - DNA, 10 bp, Artificial Sequence
  Synthetic nucleic acid sequence
gtagtgtggg

SEQ ID NO: 126 - DNA, 10 bp, Artificial Sequence
  Synthetic nucleic acid sequence
aggaggtgtt

SEQ ID NO: 127 - DNA, 10 bp, Artificial Sequence
  Synthetic nucleic acid sequence
gttggtaggt

SEQ ID NO: 128 - DNA, 10 bp, Artificial Sequence
  Synthetic nucleic acid sequence
gtaggtggtt

SEQ ID NO: 129 - DNA, 10 bp, Artificial Sequence
  Synthetic nucleic acid sequence
aggtgttggt

SEQ ID NO: 130 - DNA, 10 bp, Artificial Sequence
  Synthetic nucleic acid sequence
tatggttgtg

SEQ ID NO: 131 - DNA, 10 bp, Artificial Sequence
  Synthetic nucleic acid sequence
ttaggttagt

SEQ ID NO: 132 - DNA, 10 bp, Artificial Sequence
  Synthetic nucleic acid sequence
gattggagtt

SEQ ID NO: 133 - DNA, 10 bp, Artificial Sequence
  Synthetic nucleic acid sequence
gtagagtgga

SEQ ID NO: 134 - RNA, 24 nt, Artificial Sequence
  Synthetic nucleic acid sequence
cucuuucuuuuccauggguuggcu

SEQ ID NO: 135 - RNA, 24 nt, Artificial Sequence
  Synthetic nucleic acid sequence
ggcugagggaaggacuguccuggg

SEQ ID NO: 136 - RNA, 13 nt, Artificial Sequence
  Synthetic nucleic acid sequence
ggguuaugggacc

SEQ ID NO: 137 - RNA, 12 nt, Artificial Sequence
  Synthetic nucleic acid sequence
auauccuuuuua

SEQ ID NO: 138 - RNA, 12 nt, Artificial Sequence
  Synthetic nucleic acid sequence
guauccuuuuua

SEQ ID NO: 139 - RNA, 33 nt, Artificial Sequence
  Synthetic nucleic acid sequence
aggcuucggagcaaggaggcagcuccgaagccu

SEQ ID NO: 140 - RNA, 33 nt, Artificial Sequence
  Synthetic nucleic acid sequence
aggcuucggagcaagccuccagcuccgaagccu

SEQ ID NO: 141 - RNA, 29 nt, Artificial Sequence
  Synthetic nucleic acid sequence
gucgaggccgagcgggcaaaggccucgac

SEQ ID NO: 142 - RNA, 29 nt, Artificial Sequence
  Synthetic nucleic acid sequence
gucgaggccgagcccgcaaaggccucgac

SEQ ID NO: 143 - RNA, 10 nt, Artificial Sequence
  Synthetic nucleic acid sequence
  misc_feature (1)..(3): n is a, c, g, or u
  misc_feature (8)..(10): n is a, c, g, or u
nnnaggunnn

SEQ ID NO: 144 - RNA, 12 nt, Artificial Sequence
  Synthetic nucleic acid sequence
uuuuccuuaacu

SEQ ID NO: 145 - DNA, 1305 bp, Artificial Sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggtaagtattagctctttctttccatgggttggcctcgccgcgtgggctgagggaaggactgtcctgggactggacaggcgggttatgggacctgaaaagcggccctgaaaaagggccgcgatctgtagaaagcgagctagtgccggacagttagaggaaaaggggaagaactgtccgaaaaaagggggggaagacagtgactagaaagggaagggagaagtcactgtagaggggaaggaaaaggctagctagaggagaaggaaagaggctagctagcagaggagaaggaaaggcgccagcagttcggtgctatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 146 - DNA, 1543 bp, Artificial Sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgcggaaaaccgcgggatagcaccgaactgctggcgcctttccttctcctctgctagctagcctctttccttctcctctagctagccttttccttcccctctacagtgacttctcccttccctttctagtcactgtcttcccccccttttttcggacagttcttccccttttcctctaactgtccggcactagctcgctttctacagatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtattagctctttctttccatgggttggcctcgccgcgtgggctgagggaaggactgtcctgggactggacaggcgggttatgggacctgaaaagcggccctgaaaaagggccgcagcgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtgctaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 147 - DNA, 1571 bp, Artificial Sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccaccatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggtaagtattagctctttctttccatgggttggcctcgccgcgtgggctgagggaaggactgtcctgggactggacaggcgggttatgggacctgaaaagcggccctgaaaaagggccgcgatgaaaacgaagcgagctaaagcctcctctctcttcttcagaactcctctcttttctctcctccaggagttcttcctctctcccttcttctcaaatgctttctccctctctcctgcatttgagctccttctttcctctctcgacaatccccttttctccctcttgattgtcgactagctcgcaatcatcgcggtatcaaaaagcggtcaggcagctaaaccaaaaggtttagcaattgcctctgatgagtcgctgaaatgcgacgaaaaccgctttttggtaccaataaaatatctttattttcattacatctgtgtgttggttttttgtgtgactagt

SEQ ID NO: 148 - DNA, 1765 bp, Artificial Sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgcggaaaaccgcgggataccgcgatgattgcgagctagtcgacaatcaagagggagaaaaggggattgtcgagagaggaaagaaggagctcaaatgcaggagagagggagaaagcatttgagaagaagggagagaggaagaactcctggaggagagaaaagagaggagttctgaagaagagagaggaggctttagctcgcttcgttttcatcattattgcggccctgaaaaagggccgcttataacgttgctcgaattcgggttatgggaccagtgaaggctgagggaaggactgtcctgggactggacaggcgggttatgggacctgaaaatactaacaatcgattttttttccctttttttccaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctgagaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagt

SEQ ID NO: 149 - RNA, 10 nt, Artificial Sequence
  Synthetic nucleic acid sequence
aaagaaggaa

SEQ ID NO: 150 - RNA, 12 nt, Artificial Sequence
  Synthetic nucleic acid sequence
cuuucuuuucuu

SEQ ID NO: 151 - RNA, 11 nt, Artificial Sequence
  Synthetic nucleic acid sequence
  misc_feature (1)..(3): n is a, c, g, or u
  misc_feature (8)..(11): n is a, c, g, or u
nnnaggunnnn

SEQ ID NO: 152 - RNA, 11 nt, Artificial Sequence
  Synthetic nucleic acid sequence
  misc_feature (1)..(3): n is a, c, g, or u
  misc_feature (8)..(11): n is a, c, g, or u
nnnuggunnnn

SEQ ID NO: 153 - RNA, 11 nt, Artificial Sequence
  Synthetic nucleic acid sequence
  misc_feature (3)..(8): n is a, c, g, or u
gannnnnnaaa

SEQ ID NO: 154 - DNA, 12 bp, Artificial Sequence
  Synthetic nucleic acid sequence
gccgccaccatg

SEQ ID NO: 155 - DNA, 4311 bp, Artificial sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgccgccaccatggctctgatcgtgcacctgaaaaccgtgtccgagctgagaggcaagggcgacagaatcgccaaagtgaccttcagaggccagagcttctacagcagagtgctggaaaactgcgaaggcgtggccgacttcgacgagacattcagatggcctgtggccagcagcatcgacagaaacgaggtgctcgagatccagatcttcaactacagcaaggtgttcagcaacaagctgatcgggaccttctgcatggtgctgcagaaagtggtggaagagaaccgcgtggaagtgaccgacacactgatggacgacagcaacgccatcatcaagaccagcctgagcatggaagtgcgctaccaggccacagatggcacagtcggaccttgggacgatggcgatttcctgggagatgagagcctgcaagaggaaaaggacagccaagagacagacggcctgctgcctggctctcggcctagcacaagaatcagcggcgagaagtccttcagaagcaagggcagagaaaagaccaaaggcggcagagatggcgagcacaaggctggcagatctgtgttcagcgccatgaagctgggcaagaccagaagccacaaagaggaaccccagagacaggacgagccagccgttctggaaatggaagatctcgaccatctggccatccagctcggcgacggacttgaccctgattctgtgtctctggccagcgtgacagccctgacaagcaacgtgtccaacaagagaagcaagcccgacatcaagatggaacccagcgccggcagacccatggattaccaggtgtccatcaccgtgatcgaggccagacagctcgtgggcctgaacatggatcctgtcgtgtgtgtggaagtgggcgacgacaaaaagtacaccagcatgaaggaaagcaccaactgtccctactacaacgagtacttcgtgttcgacttccacgtgtccccagacgtgatgttcgacaagatcattaagatcagcgtgatccacagcaagaacctgctgagaagcggcacactcgtgggcagctttaagatggacgtgggcaccgtgtacagccagccagagcaccagtttcaccacaagtgggccatcctgagcgaccccgatgatatctctgctggcctgaagggctacgtgaagtgtgatgtggctgtcgtcggcaaaggcgacaacatcaagacaccccacaaggccaacgagactgacgaggacgatatcgagggcaacctgctgctgccagaaggcgtgccaccagaaagacagtgggccagattctatgtgaagatctacagagccgagggcctgcctagaatgaacacaagcctgatggccaacgtgaagaaggctttcatcggcgagaacaaggacctggtggacccctacgtccaggtgttcttcgctggacagaaaggcaagacctccgtgcagaagtccagctacgagcccctgtggaacgaacaggtggtgttcaccgatctgttccctccactgtgcaagagaatgaaggtgcagatccgggacagcgacaaagtgaacgatgtggccatcggcacccacttcatcgacctgagaaagatcagcaacgacggcgacaagggcttcctgcctacacttggacctgcctgggtcaacatgtacggcagcaccagaaactacaccctgctggacgagcaccaggacctgaacgaaggactcggagagggcgtgtccttccgggctagactgatgctgggactcgccgtggaaatcctggacacaagcaaccctgagctgaccagcagcacagaggtgcaggttgaacaggccacacctgtgtctgagagctgcaccggcagaatggaagagttcttcctgttcggcgccttcctggaagcctccatgatcgatagaaagaacggcgataagcccatcaccttcgaagtgaccatcggcaactacggcaacgaggtggacggcatgtctagacccctccggcctagaccaagaaaagagcccggcgacgaggaagaggtggacctgatccagaacagcagcgacgatgagggcgacgaagctggcgatctggcaagcgttagcagcacccctcctatgaggccccagatcaccgaccggaactactttcatctgccctacctggaaagaaagccctgcatctacatcaagagctggtggcctgaccagagaaggcggctgtacaacgctaacatcatggaccatatcgccgacaagctggaagagggactgaacgacgtccaagagatgatcaagaccgagaagtcttaccccgagagaaggctgaggggcgtgctcgaggaactgagctgtggatgccacagatttctgagcctgtccgacaaggaccagggcagaagcagcagaaccagactggatagagagcggctgaagtcctgcatgcgcgagctggaatctatgggccagcaggccaagagcctgagagcccaagtgaagagacacaccgtgcgggacaagctgagatcctgccagaacttcctgcagaagctgcggttcctggccgatgagcctcagcactctatccccgacgtgttcatctggatgatgagcaacaacaagaggatcgcctacgccagagtgcccagcaaggatctgctgtttagcatcgtggaagaggaactcggcaaggactgcgccaaagtcaagaccctgttcctgaagctgccaggcaagagaggcttcggctctgctggatggacagtgcaggctaagctggaactgtacctgtggctgggcctgagcaagcagagaaaggacttcctgtgcggcctgccttgcggcttcgaagaagtgaaggctgctcaaggcctgggcctgcacagcttccctccaatctctctggtgtacacaaagaagcaggccttccagctgagggcccacatgtaccaggctagatctctgttcgccgccgactctagcggcctgtctgatcctttcgctcgggtgttcttcatcaaccagagccagtgcaccgaggtgctgaacgagacactgtgtcctacctgggaccagatgctggtctttgacaacctcgagctgtacggcgaggctcacgaactgagagatgaccctcctatcatcgtcatcgagatctacgaccaggacagcatgggcaaagccgacttcatgggcagaaccttcgccaagcctctggtcaagatggccgacgaggcttactgccctcctcggttcccacctcagctcgagtactaccagatctaccggggctctgctacagccggcgatctgctggctgcttttgagctgctgcaaatcggccctagcggcaaggctgatctgcctccaatcaacggccctgtggacatggacagaggccccattatgcctgtgcctgtgggcatcagacccgtgctgagcaagtacagagtggaagtgctgttttggggcctgcgcgacctgaagagagtgaacctggctcaggtaagtattagctctttctttccatgggttggcctcgccgcgtgggctgagggaaggactgtcctgggactggacaggcgggttatgggacctgaagcgataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtg

SEQ ID NO: 156 - DNA, 3467 bp, Artificial sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttaggatttttgacctgctcgattgtccactgcgagcaggtcttttggagtcgggcgaggcggaagcccgactccttttggcatgcacgctagccgcgtcgtgcatgccttttatcttcgggttatgggaccagtgaaggctgagggaaggactgtcctgggactggacaggcgggttatgggacctgaaaatactaacaatcgattttttttccctttttttccaggtggacagacccagagtggatatcgagtgtgctggcaaaggggtgcagagcagcctgatccataactacaagaagaaccccaacttcaacaccctggtcaagtggttcgaagtggatctgcccgagaacgaactgctgcacccacctctgaacatcagagtggtggactgcagagccttcggcagatacaccctcgtgggatctcacgccgtgtctagcctgagaagattcatctacagacctccagacagaagcgcccctaactggaacacaacaggcgaggtggtggtgtccatggaacccgaggaacccgtgaagaaactggaaaccatggtcaagctggacgccacctccgatgctgtcgtgaaagtggacgtggccgaggacgagaaagagcgcaagaagaagaaaaagaagggccccagcgaggaacctgaagaggaagaacctgacgagagcatgctggactggtggtccaagtacttcgcctccatcgacacaatgaaggaacagctgagacagcacgagacaagcggcaccgacctcgaagagaaagaagagatggaatccgccgaaggactgaagggccctatgaagtccaaagagaagtctagggccgccaaagaagagaaaaaaaagaagaaccagtctcctggaccaggccagggatctgaggctcccgaaaagaaaaaggccaagatcgacgagctgaaggtgtaccccaaagagctggaaagcgagttcgacagcttcgaggactggctgcacaccttcaatctgctgagaggaaagacaggcgacgacgaggatggcagcactgaagaagagagaatcgtcggcagattcaagggcagcctgtgcgtgtacaaggtgccactgcctgaggacgtgtccagagaggctggctacgatcctacctacggcatgttccaaggcatccctagcaacgaccccatcaatgtgctcgtgcggatctatgtcgtgcgggccactgatctgcatcccgccgatatcaacggcaaggcagacccctatatcgctatcaagctggggaaaaccgacatcagggacaaagagaactacatcagcaagcagctgaaccccgtgttcggcaagagcttcgacatcgaggctagcttccccatggaatccatgctgaccgtggccgtgtacgactgggatctcgtgggaacagacgacctgatcggagagacaaagattgacctggaaaaccggttctactccaagcaccgggccacctgtggaatcgcccagacctactctatccacggctacaacatctggcgggaccccatgaagcctagccagatcctgaccaggctgtgcaaagaaggcaaggtcgacggccctcactttggacctcacggccgggtcagagtggccaacagagtgttcacaggcccctccgagatcgaggatgagaacggccagagaaagcccaccgatgagcatgtggctctgagcgctctgagacactgggaagatatccctagagtgggctgcagactggtgcccgagcacgtggaaacaagacccctgctgaacccagacaagcccggaatcgaacagggcagactcgaactgtgggtcgacatgttccctatggacatgcccgcacctggcacaccactggacatcagccctaggaagcccaagaaatacgagctgcgcgtgatcgtgtggaacaccgacgaagtggtgctggaagatgacgacttcttcaccggcgaaaagtccagcgacatcttcgtcagaggatggctgaagggacagcaagaggataagcaggacaccgacgtgcactaccacagccttacaggcgaaggcaactttaactggcgctacctgtttcctttcgactacctggccgccgaagagaagatcgtgatgtccaagaaagaatctatgttcagctgggacgagacagagtacaagatccccgccagactgaccctgcagatctgggatgccgatcacttcagcgccgacgactttctgggagccatcgagctggacctgaatagattccccagaggcgccaagaccgccaagcagtgcacaatggaaatggccactggcgaggtcgacgtgccactggtgtctatcttcaagcagaagcgcgtcaaaggctggtggcccctgctggctagaaacgagaacgacgagttcgagctgaccggaaaggtggaagccgagctgcatctgctgacagctgaagaggccgagaagaatcctgtgggcctcgctaggaatgagcccgatcctctggaaaagcccaacagacccgataccgccttcgtgtggtttctgaacccactgaagtccatcaagtacctgatctgtacccggtacaagtggctgattatcaagatcgtgctggccctgctggggctgctgatgcttgctctgttcctgtactccctgcctggctatatggtcaagaagctgctgggcgccggcgctcgggctgactacaaagaccatgacggtgattataaagatcatgacatcgactataaggatgacgatgacaaatgaggtaccaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 157 - DNA, 4392 bp, Artificial sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgccaccatggtcattctgcagcagggcgaccacgtgtggatggatctgagactgggccaagagttcgacgtgccaatcggcgccgtggtcaagctgtgtgattctggccaggtgcaagtcgtggacgacgaggataatgagcactggatcagccctcagaacgccacacacatcaagcctatgcaccccacatctgtgcacggcgtggaagatatgatccggctgggcgatctgaacgaggccggcatcctgagaaacctgctgatcagataccgggaccacctgatctacacctacaccggctctatcctggtggccgtgaatccctaccagctgctgagcatctacagccccgagcacatccggcagtacaccaacaagaaaatcggcgagatgcctcctcacatcttcgccattgccgacaactgctacttcaacatgaagcggaacagccgggaccagtgctgcatcatctctggcgaatctggcgccggaaagaccgagagcacaaagctgatcctgcagttcctggccgccatcagcggacagcactcttggattgagcagcaggtcctggaagccacacctattctggaagccttcggcaacgccaagaccatccggaacgacaacagcagcagattcggcaaatacatcgacatccacttcaacaagagaggcgccattgagggcgccaagatcgagcagtacctgctggaaaagtccagagtgtgcagacaggccctggacgagagaaactaccacgtgttctactgcatgctggaaggcatgagcgaggaccagaagaagaagctcggactcggccaggccagcgactacaattatctggccatgggcaactgcatcacatgcgagggcagagtggacagccaagagtacgccaacatccgcagcgccatgaaggtgctgatgttcaccgacaccgagaactgggagatcagcaaactgctggccgctatcctgcatctgggcaacctgcagtacgaggccagaaccttcgagaacctggatgcctgcgaggtgctgttctctccttccctggctaccgccgcctctctgctggaagtgaaccctcctgatctgatgagctgcctgaccagcagaaccctgatcaccagaggcgagacagtgtctacccctctgagcagagaacaggctctggatgtgcgggacgccttcgtgaagggcatctacggcagactgttcgtgtggatcgtggacaagatcaacgccgccatctacaagcctccaagccaggacgtgaagaacagcagaagatccatcggcctgctggacatcttcggcttcgagaatttcgccgtgaacagcttcgagcagctgtgcatcaacttcgccaacgagcacctccagcagttcttcgtgcggcacgtgttcaagctggaacaagaggaatacgacctggaatccatcgactggctgcacatcgagttcaccgataaccaggacgccctggacatgatcgccaacaagcccatgaacatcatcagcctgatcgacgaggaaagcaagttccccaagggcaccgataccaccatgctgcacaagctgaacagccagcacaaactgaatgccaactacatcccgcctaagaacaaccacgagacacagttcggcatcaaccacttcgccggcatcgtgtactacgaaacccagggctttctggaaaagaaccgggacaccctgcacggcgacatcattcagctggtgcacagcagccggaacaagttcatcaagcagatcttccaggccgacgtcgccatgggagccgagacaagaaagagaagccccacactgagcagccagttcaagcggagtctggaactgctgatgagaaccctgggagcctgccagcctttctttgtgcggtgcatcaagcccaacgagttcaagaaacccatgctgttcgaccggcacctgtgtgtgcggcagctgagatacagcggcatgatggaaaccatcaggattcggagagccggctatcccatccggtacagcttcgtggaattcgtcgagcggtacagagtgctgctgcctggcgtgaagcctgcctacaaacagggcgatctcagaggcacctgtcagagaatggccgaagccgtgctgggcacccatgacgattggcagatcggaaagacaaagatcttcctgaaggaccaccacgacatgctgctcgaggtggaaagagacaaggccatcaccgacagagtgatcctgctccagaaagtgatccggggcttcaaggacagaagcaatttcctgaagctgaagaatgccgccactctgatccagagacactggcggggacacaactgccggaagaactacggcctgatgaggctgggcttcctgagactgcaggccctgcacagaagcagaaagctgcaccagcagtacagactggcccggcagcggatcatccagtttcaagccagatgtcgggcctacctcgtgcgcaaggccttcagacatagactgtgggccgtgctgaccgtgcaggcctatgccagaggaatgattgcccgcagactgcaccagagactgagagccgagtatctgtggcggctggaagccgagaaaatgcggctggccgaggaagagaagctgcggaaagagatgagcgccaagaaggccaaagaagaggccgagcggaagcaccaagagagactggctcaactggccagagaggacgccgagagagagctgaaagagaaagaggccgccagacggaagaaagaactcctggaacagatggaacgggccagacacgagcccgtgaaccacagcgatatggtggataagatgttcggcttcctgggcacctctggcggactgcctggacaagaaggacaggcccctagcggctttgaggacctggaacgtgggagaagagaaatggtggaagaggatctggacgccgctctgcctctgcctgacgaggatgaagaagatctgagcgagtacaagttcgccaagtttgccgccacctactttcaaggcaccaccacacacagctacaccagaaggcctctgaagcagcccctgctgtaccacgatgatgagggcgatcaactggcagccctggccgtgtggattaccatcctcagattcatgggcgacctgcctgagcctaagtaccacaccgccatgtctgacggctccgagaagatccccgtgatgaccaagatctacgagactctgggcaagaaaacctacaagcgcgagctgcaggctctccaaggcgaaggcgaagctcaactgcctgagggccagaaaaagtcctctgtgcgccacaaactggtgcacctgacactgaagaagaaaagcaagctgacagaggaagtgaccaagcggctgcacgatggcgagtctacagtgcagggcaacagcatgctcgaggacagacccaccagcaacctggaaaaactgcacttcatcatcggcaacggaatcctgcggcctgctctgagggatgagatctactgccagatctccaagcagctgacacacaaccccagcaagagcagctacgccagaggctggattctggtaagtattagctctttctttccatgggttggcctcgccgcgtgggctgagggaaggactgtcctgggactggacaggcgggttatgggacctgaagcgataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtg

SEQ ID NO: 158 - DNA, 4055 bp, Artificial sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttaggatttttgacctgctcgattgtccactgcgagcaggtcttttggagtcgggcgaggcggaagcccgactccttttggcatgcacgctagccgcgtcgtgcatgccttttatcttcgggttatgggaccagtgaaggctgagggaaggactgtcctgggactggacaggcgggttatgggacctgaaaatactaacaatcgattttttttccctttttttccaggtgtctctgtgcgtgggctgtttcgccccaagcgagaagttcgtgaagtacctgaggaacttcatccacggcggacctccaggctacgccccttactgtgaagagaggctgagaaggacctttgtgaacggcacccggacacagcctccatcctggctggaactccaggccaccaagagcaaaaagcccatcatgctgcccgtgacctttatggatggcaccacaaagaccctgctgaccgatagcgccaccaccgccaaagagctgtgtaacgccctggctgacaagattagcctgaaggatagattcggcttcagcctgtacattgccctgttcgacaaggtgtccagcctcggctctggctctgaccatgtgatggatgccatcagccagtgcgagcagtatgccaaagaacagggcgcccaagagaggaacgctccttggcggctgttctttcggaaagaggtgttcaccccttggcacagccccagcgaagataacgtggccaccaatctgatctaccagcaagttgtgcggggcgtgaagttcggcgagtacagatgcgaaaaagaggacgatctggccgagctggcctctcagcagtactttgtggactacggcagcgagatgatcctggaacggctgctgaatctggtgcccacctacattcccgatcgggagatcaccccactgaaaaccctcgagaagtgggcccagctggccattgctgcccacaagaaaggcatctatgcccagcggagaacagacgcccagaaagtcaaagaggatgtcgttagctacgcccggttcaagtggcctctgctgtttagccggttctacgaggcctacaagttcagcggccccagtctgcccaagaacgatgtgatcgtggctgtgaactggaccggcgtgtacttcgtggatgagcaagaacaagtgctgcttgagctgagcttccccgagatcatggccgtgtccagctccagagaatgcagagtgtggctgagcctgggctgtagcgatctgggatgtgccgctcctcattctggatgggctggactgacaccagccggaccttgtagcccttgttggtcttgccggggggccaagacaacagcccctagctttaccctggccaccattaagggcgacgagtacaccttcaccagcagcaacgccgaggacatcagagatctggtcgtgaccttcctggaaggcctgcggaagcggagcaaatatgtggtggccctgcaggacaaccccaatcctgctggcgaggaatccggctttctgagctttgccaaaggcgacctgatcatcctggaccacgacaccggcgagcaagtgatgaatagcggctgggccaacggcatcaatgagcggacaaagcagcggggcgacttccctaccgatagcgtgtacgtgatgcccaccgtgaccatgcctccaagggaaatcgtggccctggtcaccatgacacccgaccagagacaggatgttgtgcggctgctgcagctgaggacagccgaaccagaagtgcgggccaagccttacacactggaagagttcagctacgactacttccggcctcctccaaagcacaccctgtctagagtgatggtgtccaaggccagaggcaaggataggctgtggtcccacacaagagagcccctgaaacaggcactgctgaaaaagctgctgggcagcgaggaactgagccaagaagcctgtctggcctttatcgccgtgctgaagtacatgggcgattacccctccaagcggaccagatccgtgaacgaactgaccgaccagattttcgagggcccactgaaggccgagcctctgaaagatgaggcctacgtgcagattctgaaacagctgaccgacaaccacatccgctacagcgaggaacgcggatgggaactgctgtggctgtgtaccggactgttcccacctagcaacattctgctgccccacgtgcagcggtttctgcagtctagaaagcactgccctctggccatcgattgcctgcagaggctgcaaaaggccctgagaaatggctcccggaagtaccctcctcacctggtggaagtggaagccatccagcacaagaccacacagatctttcacaaggtctacttccccgacgacacagacgaggcctttgaggtggaatcctctaccaaggccaaggacttctgccagaatatcgccaccaggctgctgctgaagtccagcgaaggctttagcctgtttgtgaagatcgccgacaaagtgctgagcgtgcccgagaacgacttctttttcgattttgtgcgccatctgaccgactggattaagaaggctagacccatcaaggatggcatcgtgcccagcctgacctatcaggtgttctttatgaagaagctgtggacgaccaccgtgcctggcaaggatcctatggccgacagcatcttccactactaccaagagctgcccaagtacctgcggggctaccacaagtgtaccagagaagaggtcctgcagctgggagccctgatctatagagtgaagtttgaagaggacaagagctacttccctagcatccccaagctgctgcgcgaactggttccccaggatctgatccggcaagtgtcccctgatgactggaagcggtctatcgtggcctactttaacaagcacgccggcaagagtaaagaggaagccaagctggcctttctgaagctcatctttaagtggcctaccttcggctccgccttcttcgaagtgaagcagaccaccgagcctaacttccctgagattctgctgatcgccatcaacaaatacggcgtgtccctgatcgatcccaagacaaaggacatcctgacaacacaccccttcaccaaaatcagcaactggtccagcggcaacacctacttccacatcaccatcggcaatctcgtgcggggctctaagctgctgtgtgaaaccagcctgggatacaagatggacgacctgctgacaagctacatctcccagatgctgaccgccatgagcaaacagagaggctctcggagcggcaagtggggcgctcgggctgactacaaagaccatgacggtgattataaagatcatgacatcgactataaggatgacgatgacaaatgaggtaccaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 159 - DNA, 4161 bp, Artificial sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgccaccatgaagagaaccgccgacggcagcgagttcgagagccctaagaaaaagcggaaggtggacaagaagtacagcatcggcctggctatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagaggacagccagacggcggtacaccagaagaaagaaccggatctgctacctgcaagagatcttcagcaacgagatggccaaggtggacgacagcttcttccaccggctggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgcccggctgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccaaatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggataagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatcggaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccaaaacggccgggatatgtatgtggaccaagagctggacatcaaccggctgtccgactacgatgtggacgctatcgtgccccagtcttttctgaaagacgactccatcgacaacaaggtcctgaccagaagcgacaagaaccggggcaagagcgataacgtgccctccgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaatccgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggacaagggcagagattttgccaccgtgcggaaagtgctgagcatgccccaagtgaatatcgtgaaaaagaccgaggtaagtattagctctttctttccatgggttggcctcgccgcgtgggctgagggaaggactgtcctgggactggacaggcgggttatgggacctgaagcgataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtg

SEQ ID NO: 160 - DNA, 3410 bp, Artificial sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttaggatttttgacctgctcgattgtccactgcgagcaggtcttttggagtcgggcgaggcggaagcccgactccttttggcatgcacgctagccgcgtcgtgcatgccttttatcttcgggttatgggaccagtgaaggctgagggaaggactgtcctgggactggacaggcgggttatgggacctgaaaatactaacaatcgattttttttccctttttttccaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatctccgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagctcggaggcgatagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttgaggtaccaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 161 - DNA, 4161 bp, Artificial sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgccaccatgaagagaaccgccgacggcagcgagttcgagagccctaagaaaaagcggaaggtggacaagaagtacagcatcggcctggacatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagaggacagccagacggcggtacaccagaagaaagaaccggatctgctacctgcaagagatcttcagcaacgagatggccaaggtggacgacagcttcttccaccggctggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgcccggctgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccaaatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggataagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatcggaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccaaaacggccgggatatgtatgtggaccaagagctggacatcaaccggctgtccgactacgatgtggacgctatcgtgccccagtcttttctgaaagacgactccatcgacaacaaggtcctgaccagaagcgacaagaaccggggcaagagcgataacgtgccctccgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaatccgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggacaagggcagagattttgccaccgtgcggaaagtgctgagcatgccccaagtgaatatcgtgaaaaagaccgaggtaagtattagctctttctttccatgggttggcctcgccgcgtgggctgagggaaggactgtcctgggactggacaggcgggttatgggacctgaagcgataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtg

SEQ ID NO: 162 - DNA, 3911 bp, Artificial sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttaggatttttgacctgctcgattgtccactgcgagcaggtcttttggagtcgggcgaggcggaagcccgactccttttggcatgcacgctagccgcgtcgtgcatgccttttatcttcgggttatgggaccagtgaaggctgagggaaggactgtcctgggactggacaggcgggttatgggacctgaaaatactaacaatcgattttttttccctttttttccaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatctccgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagctcggaggcgattctggcggatctagcggtggaagctctggctctgagacacctggcacaagcgagtctgccacacctgagtctagcggcggatcttcaggcggcagcagcaccctgaatatcgaggatgagtacagactgcacgagacaagcaaagaacccgacgtgtccctgggctctacctggctgtctgattttcctcaagcctgggccgaaacaggcggaatgggacttgctgttagacaggctcccctgatcattcccctgaaggccacaagcacccctgtgtccatcaagcagtaccccatgtctcaagaggcccggctgggaatcaagccccacattcagagactgctggaccagggcatcctggtgccttgtcaaagcccttggaatacccctctgctgcctgtgaagaagcccggcaccaacgactacagacccgtgcaggatctgcgcgaagtgaacaagagagtcgaggacattcaccccaccgtgcctaatccttacaacctgctgtctggcctgcctccttcccaccaatggtacacagtgctggacctgaaggatgccttcttctgcctgcggctgcaccctacaagccagcctctgtttgccttcgagtggcgggatccagagatgggcattagcggacagctgacctggaccagactgccccagggcttcaagaatagccccacactgttcaacgaggccctgcacagggacctcgccgactttagaattcagcaccccgacctgattctgctgcagtatgtggatgatctgctgctggccgctaccagcgagctggattgtcagcagggaacaagagccctgctgcagaccctgggcaatctgggctatagagcctctgccaagaaggcccagatttgccagaagcaagttaagtacctgggctacctgctcaaagaaggccagcgttggctgaccgaggccagaaaagaaaccgtgatgggccagcctacacctaagacacccagacagctgagagagttcctgggcaaagccggattctgcaggctgtttatccctggcttcgccgagatggctgcccctctgtatcctctgacaaagcccggaactctgttcaactggggcccagaccagcagaaagcctaccaagagatcaagcaggctctgctgacagcccctgctctgggactgcctgatctgaccaagcctttcgagctgttcgtggacgagaagcagggctatgccaagggcgtgctgacacagaaactcggcccttggagaaggcccgtggcttacctgagcaaaaagctggatcctgtggccgctggctggcctccttgtctgagaatggtggccgctatcgccgtgctgactaaggatgccggcaagctgacaatgggacagcctctggttattctggcccctcatgccgtggaagccctcgtgaaacagcctcctgatcggtggctgagcaacgccagaatgacccactaccaggcactgctgctcgacaccgacagagtgcaatttggccctgtggtggccctgaatccagccacattgctgcctctgcctgaggagggactgcagcacaactgcctcgatatcctggctgaggcccacggcacaagacccgatctgacagatcagccactgcctgacgccgaccacacctggtatacagatggcagctctctgctgcaagagggccagagaaaagctggcgccgctgtgaccacagagacagaagtgatttgggccaaagctctgcctgccggcacatctgcccaaagagccgaactgatcgcactgacacaggccctgaagatggccgagggcaagaaactgaacgtgtacaccgactccagatacgccttcgccaccgctcacatccacggcgaaatctacagacgcagaggatggctgaccagcgagggaaaagagattaagaacaaggacgagattctcgccctcctcaaggccctgttcctgcctaagcggctgagcatcatccactgtcctggccaccagaagggacactctgccgaggctagaggcaacagaatggccgatcaggctgccagaaaggccgccattaccgagacacccgataccagcacactgctgattgagaacagcagcccttccggcggctccaaaagaacagctgacggctccgagtttgagcccaaaaagaaacggaaagtgtgaggtaccaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 163 - DNA, 3159 bp, Artificial sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccaccatgaaacggacagccgacggaagcgagttcgagtcaccaaagaagaagcggaaagtcagcagtgaaaccggaccagtggcagtggacccaaccctgaggagacggattgagccccatgaatttgaagtgttctttgacccaagggagctgaggaaggagacatgcctgctgtacgagatcaagtggggcacaagccacaagatctggcgccacagctccaagaacaccacaaagcacgtggaagtgaatttcatcgagaagtttacctccgagcggcacttctgcccctctaccagctgttccatcacatggtttctgtcttggagcccttgcggcgagtgttccaaggccatcaccgagttcctgtctcagcaccctaacgtgaccctggtcatctacgtggcccggctgtatcaccacatggaccagcagaacaggcagggcctgcgcgatctggtgaattctggcgtgaccatccagatcatgacagccccagagtacgactattgctggcggaacttcgtgaattatccacctggcaaggaggcacactggccaagatacccacccctgtggatgaagctgtatgcactggagctgcacgcaggaatcctgggcctgcctccatgtctgaatatcctgcggagaaagcagccccagctgacatttttcaccattgctctgcaatcttgtcactatcagcggctgcctcctcatattctgtgggctaccggcctgaagtctggaggatctagcggaggatcctctggcagcgagacaccaggaacaagcgagtcagcaacaccagagagcagtggcggcagcagcggcggcagcgacaagaagtacagcatcggcctggccatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagaggacagccagacggcggtacaccagaagaaagaaccggatctgctacctgcaagagatcttcagcaacgagatggccaaggtggacgacagcttcttccaccggctggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgcccggctgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccaaatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggataagggcgccagcgctcagtccttcatcgagcggatgaccaacttcgataagaacctgcctaacgagaaggtaagtattagctctttctttccatgggttggcctcgccgcgtgggctgagggaaggactgtcctgggactggacaggcgggttatgggacctgaagcgataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtg

SEQ ID NO: 164 - DNA, 4115 bp, Artificial sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttaggatttttgacctgctcgattgtccactgcgagcaggtcttttggagtcgggcgaggcggaagcccgactccttttggcatgcacgctagccgcgtcgtgcatgccttttatcttcgggttatgggaccagtgaaggctgagggaaggactgtcctgggactggacaggcgggttatgggacctgaaaatactaacaatcgattttttttccctttttttccaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatcggaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccaaaacggccgggatatgtatgtggaccaagagctggacatcaaccggctgtccgactacgatgtggaccatatcgtgccccagtcttttctgaaagacgactccatcgacaacaaggtcctgaccagaagcgacaagaaccggggcaagagcgataacgtgccctccgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaatccgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggacaagggcagagattttgccaccgtgcggaaagtgctgagcatgccccaagtgaatatcgtgaaaaagaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatctccgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagctcggaggcgatagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaaccaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 165 - DNA, 2973 bp, Artificial sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccaccatgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagaggacagccagacggcggtacaccagaagaaagaaccggatctgctacctgcaagagatcttcagcaacgagatggccaaggtggacgacagcttcttccaccggctggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgcccggctgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccaaatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggataagggcgccagcgctcagtccttcatcgagcggatgaccaacttcgataagaacctgcctaacgagaaggtaagtattagctctttctttccatgggttggcctcgccgcgtgggctgagggaaggactgtcctgggactggacaggcgggttatgggacctgaagcgataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtg

SEQ ID NO: 166 - DNA, 3560 bp, Artificial sequence
  Synthetic nucleic acid sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttaggatttttgacctgctcgattgtccactgcgagcaggtcttttggagtcgggcgaggcggaagcccgactccttttggcatgcacgctagccgcgtcgtgcatgccttttatcttcgggttatgggaccagtgaaggctgagggaaggactgtcctgggactggacaggcgggttatgggacctgaaaatactaacaatcgattttttttccctttttttccaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatcggaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccaaaacggccgggatatgtatgtggaccaagagctggacatcaaccggctgtccgactacgatgtggaccatatcgtgccccagtcttttctgaaagacgactccatcgacaacaaggtcctgaccagaagcgacaagaaccggggcaagagcgataacgtgccctccgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacactaagtacgacgagaatgacaagctgatccgggaagtgaaagtgatcaccctgaagtccaagctggtgtccgatttccggaaggatttccagttctacaaagtgcgcgagatcaacaactaccatcacgcccacgacgcctacctgaatgccgttgttggaacagccctgatcaagaagtatcccaagctggaatccgagttcgtgtacggcgactacaaggtgtacgacgtgcggaagatgatcgccaagagcgagcaagagattggcaaggctaccgccaagtacttcttctacagcaacatcatgaactttttcaagaccgagattaccctggccaacggcgagatcagaaagcggcctctgatcgagacaaacggcgaaaccggcgagattgtgtgggacaagggcagagattttgccaccgtgcggaaagtgctgagcatgccccaagtgaatatcgtgaaaaagaccgaggtgcagacaggcggcttcagcaaagagtccattctgcccaagagaaacagcgataagctgatcgcccggaagaaggactgggaccctaagaagtacggcggcttcgatagccctaccgtggcctattctgtgctggtggtggccaaagtggaaaagggcaagtccaagaaactcaagagcgtgaaagagctgctggggatcaccatcatggaaagaagcagcttcgagaagaatcctatcgatttcctcgaggccaagggctacaaagaagtgaaaaaggacctgatcatcaagctccccaagtactccctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatctccgagttctccaagagagtgatcctggccgacgctaatctggacaaagtgctgtccgcctacaacaagcaccgggacaagcctatcagagagcaggccgagaatatcatccacctgtttaccctgaccaatctgggagcccctgccgccttcaagtactttgacaccaccatcgaccggaagcgctacaccagcaccaaagaggtgctggacgccacactgatccaccagtctatcaccggcctgtacgagacacggatcgacctgtctcagctcggaggcgattctggcggctcaaaaagaaccgccgacggcagcgaattcgagcccaagaagaagaggaaagtctaaggtaccaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 167 - DNA, 112 bp, Artificial sequence
  Synthetic nucleic acid sequence
gatttttgacctgctcgattgtccactgcgagcaggtcttttggagtcgggcgaggcggaagcccgactccttttggcatgcacgctagccgcgtcgtgcatgccttttatc

SEQ ID NO: 168 - DNA, 13 bp, Artificial sequence
  Synthetic nucleic acid sequence
gggttatgggacc

SEQ ID NO: 169 - DNA, 24 bp, Artificial sequence
  Synthetic nucleic acid sequence
ggctgagggaaggactgtcctggg

SEQ ID NO: 170 - DNA, 24 bp, Artificial sequence
  Synthetic nucleic acid sequence
ctctttctttccatgggttggcct

SEQ ID NO: 171 - DNA, 4463 bp, Artificial sequence
  Synthetic nucleic acid sequence
  misc_feature (4225)..(4294): n is a, c, g, or t
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgccggaagcggagctactaacttcagcctgctgaagcaggctggagacgtggaggagaaccctggacctaggcgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaagnnnnnnnnnnnnnnnnnnnnnnnnnnnnnnnnnnnnnnnnnnnnnnnnnnnnnnnnnnnnnnnnnnnnnngataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtg

SEQ ID NO: 172 - DNA, 3467 bp, Artificial sequence; identical to SEQ ID NO: 156
  Synthetic nucleic acid sequence

SEQ ID NO: 173 - DNA, 33 bp, Artificial sequence
  Synthetic nucleic acid sequence
gtaagtattgctttcatttttgtctttttttaa

SEQ ID NO: 174 - DNA, 30 bp, Artificial sequence
  Synthetic nucleic acid sequence
gtaagttcttgctttgttcaaactgtctat

SEQ ID NO: 175 - DNA, 27 bp, Artificial sequence
  Synthetic nucleic acid sequence
gtaagtattcttttgttcttcactcat

SEQ ID NO: 176 - DNA, 32 bp, Artificial sequence
  Synthetic nucleic acid sequence
gtaagtatttttttactcctcatttttactcc

SEQ ID NO: 177 - DNA, 36 bp, Artificial sequence
  Synthetic nucleic acid sequence
gtaagtatttttttacggttatattctcctttcccc

SEQ ID NO: 178 - DNA, 28 bp, Artificial sequence
  Synthetic nucleic acid sequence
gtaagtattttctgttgtttattttcag

SEQ ID NO: 179 - DNA, 39 bp, Artificial sequence
  Synthetic nucleic acid sequence
gtaagtattggggttgattatgtgtgggacggtgtaagg

SEQ ID NO: 180 - DNA, 35 bp, Artificial sequence
  Synthetic nucleic acid sequence
gtaagtatttcctctttctttccatgggttggcct

SEQ ID NO: 181 - DNA, 35 bp, Artificial sequence
  Synthetic nucleic acid sequence
gtaagtattaccagagattcgtagacctgcttgac

SEQ ID NO: 182 - DNA, 39 bp, Artificial sequence
  Synthetic nucleic acid sequence
tggggctgggcagagggttgaggggagagggtcctgggg

SEQ ID NO: 183 - DNA, 28 bp, Artificial sequence
  Synthetic nucleic acid sequence
tcatgggtgggttcattgggtgggttca

SEQ ID NO: 184 - DNA, 23 bp, Artificial sequence
  Synthetic nucleic acid sequence
tagggcgcagtagtccagggttt

SEQ ID NO: 185 - DNA, 30 bp, Artificial sequence
  Synthetic nucleic acid sequence
ttctctgtggggtggcattctctgctctct

SEQ ID NO: 186 - DNA, 29 bp, Artificial sequence
  Synthetic nucleic acid sequence
gggttatgggacctcagggataagggacc

SEQ ID NO: 187 - DNA, 15 bp, Artificial sequence
  Synthetic nucleic acid sequence
cggggatgggggtca

SEQ ID NO: 188 - DNA, 23 bp, Artificial sequence
  Synthetic nucleic acid sequence
tggggggaggtcatggggggagg

SEQ ID NO: 189 - DNA, 24 bp, Artificial sequence
  Synthetic nucleic acid sequence
gttggtggtttcatgttggtggtt

SEQ ID NO: 190 - DNA, 29 bp, Artificial sequence
  Synthetic nucleic acid sequence
gggtttcgggttttcaggtggtcgttggt

SEQ ID NO: 191 - DNA, 29 bp, Artificial sequence
  Synthetic nucleic acid sequence
ggtggtcgttggttcatttgggctattgg

SEQ ID NO: 192 - DNA, 29 bp, Artificial sequence
  Synthetic nucleic acid sequence
tttgggctattggtcaagggggcgagggg

SEQ ID NO: 193 - DNA, 29 bp, Artificial sequence
  Synthetic nucleic acid sequence
agggggcgaggggtcaggtattcggtatt

SEQ ID NO: 194 - DNA, 29 bp, Artificial sequence
  Synthetic nucleic acid sequence
ggtattcggtatttcaaggtaacaggtaa

SEQ ID NO: 195 - DNA, 29 bp, Artificial sequence
  Synthetic nucleic acid sequence
aggtaacaggtaatcagggtttcgggttt

SEQ ID NO: 196 - DNA, 29 bp, Artificial sequence
  Synthetic nucleic acid sequence
tcttacttttgtaaactttatggtttgtg

SEQ ID NO: 197 - DNA, 28 bp, Artificial sequence
  Synthetic nucleic acid sequence
cacgtattctcggtacggacgttacaga

SEQ ID NO: 198 - DNA, 13 bp, Artificial sequence
  Synthetic nucleic acid sequence
taagctggtatcc

SEQ ID NO: 199 - DNA, 34 bp, Artificial sequence
  Synthetic nucleic acid sequence
cactaactctttttccccccttttttttttacag

SEQ ID NO: 200 - DNA, 36 bp, Artificial sequence
  Synthetic nucleic acid sequence
tactaactctttcttttttcctttccttcttcacag

SEQ ID NO: 201 - DNA, 43 bp, Artificial sequence
  Synthetic nucleic acid sequence
cactaactctgtcatacttatcctgtcccttttttttccacag

SEQ ID NO: 202 - DNA, 45 bp, Artificial sequence
  Synthetic nucleic acid sequence
cactaactctctttctttttcttccctcctctcccccaactgcag

SEQ ID NO: 203 - DNA, 38 bp, Artificial sequence
  Synthetic nucleic acid sequence
cactaactcttttttttttttttttttttttacagcag

SEQ ID NO: 204 - DNA, 13 bp, Artificial sequence
  Synthetic nucleic acid sequence
taagctggtatcc

SEQ ID NO: 205 - DNA, 8 bp, Artificial Sequence
  branch point sequence
tactaaca

SEQ ID NO: 206 - DNA, 49 bp, Artificial Sequence
  polyadenylation signal
aataaaatatctttattttcattacatctgtgtgttggttttttgtgtg

SEQ ID NO: 207 - DNA, 4104 bp, Streptococcus pyogenes
  CDS (1)..(4104)
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 208 - protein, 1368 aa, Streptococcus pyogenes [sequence omitted]

SEQ ID NO: 209 - DNA, 4104 bp, Artificial Sequence
  dCas9
  CDS (1)..(4104)
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 210 - protein, 1368 aa, Artificial Sequence
  Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 211 - DNA, 2904 bp, Ruminococcus flavefaciens
  CDS (1)..(2904)
atgatcgaaaagaagaagtcatttgcaaagggcatgggagtaaaatcaacacttgtatccggttcaaaggtatacatgacgacgttcgcagaaggaagcgatgccagacttgaaaagatcgttgaaggcgattctatcagatctgtcaacgaaggagaagcgttctcagctgaaatggctgataagaatgcaggctacaagatcggtaacgcaaagttcagccacccaaagggctatgctgtagttgcaaacaaccccttatacaccggaccggtacagcaggatatgctcggtctgaaggaaacgcttgaaaagagatattttggagagtctgccgacggaaatgataatatctgtattcaggtcatccataatatcctcgatatcgaaaagatcctcgctgaatatataaccaatgctgcttatgcggtaaacaatatttccggtcttgataaggatatcatcggttttggtaagttcagtacggtctatacttatgatgagttcaaggatcctgaacatcacagagcagctttcaacaataacgataagttaattaatgccatcaaggcacagtatgatgaatttgacaatttccttgataatcctcgtctcggctactttggacaggcttttttcagtaaggaaggcagaaattacattatcaattacggcaacgagtgttatgatattcttgctttactcagcggattgcgtcactgggtagtacataataatgaggaagaatcaaggatttcccgtacatggctttataatctcgacaagaatcttgacaacgaatatatctctactctcaattatctgtatgatagaattacaaacgaattaacaaattccttctcaaagaatagtgcagccaacgtaaactatatcgctgaaacccttggtattaatcctgctgaatttgcagagcagtatttcagattcagtatcatgaaggaacagaagaatctcggtttcaatattactaagctgagagaagtaatgcttgacagaaaggatatgtctgagatccgtaaaaatcataaggtctttgattcaatccgtactaaggtctatactatgatggatttcgttatctacagatattacattgaagaggatgcaaaggttgctgctgccaacaagtctctgccggataacgaaaaaagcctcagtgaaaaggatatctttgttataaatctcagaggaagctttaacgatgatcagaaggatgccctttattatgatgaggccaatcgtatttggagaaagctcgaaaacattatgcacaatatcaaggaattcagaggcaataagacacgtgaatacaagaagaaggatgctccaagactccccagaattcttcctgccggaagggatgtttccgcgttctcaaagttgatgtacgctcttaccatgttccttgatggtaaggagatcaatgatcttctcaccacgctcatcaataagttcgataacatccagagtttcctcaaggtaatgcctcttatcggagtgaatgcaaagtttgttgaggaatatgccttcttcaaggacagcgcaaagattgctgacgaactcaggctgattaagagctttgccagaatgggagaacctatcgcagatgcaagacgtgctatgtatatcgatgctatcaggattctcggaacaaacctcagctatgatgagcttaaggcccttgccgatactttttcgcttgatgaaaacggcaacaagcttaagaagggcaagcacggcatgagaaacttcatcattaataatgtaatcagtaacaagcgcttccattatctcattcgttacggtgatcctgcacatctccatgagatcgccaagaatgaagctgttgtaaagttcgtcctcggcaggatagctgatatccagaagaagcagggacagaacggaaagaatcagatcgacaggtactatgagacctgtatcggcaaggacaagggcaagtctgtctccgaaaaggttgatgccctcacaaagattatcaccggtatgaactacgatcagttcgataagaagagaagcgttattgaggatactggaagagaaaacgctgagagagaaaagttcaagaagatcatcagcctctatcttactgtcatttatcacatccttaagaatattgttaatatcaatgcgcgttacgttatcggcttccattgcgttgagcgtgatgcacagctctataaggaaaagggctatgatatcaacctcaagaagctcgaagaaaaggggttttcatcagtcacaaagctgtgtgcaggtattgatgagactgctcctgacaagcgtaaggatgttgaaaaggaaatggctgagcgtgcaaaggaatctatcgatagccttgaatctgcaaatcctaagctttacgcaaactatatcaagtattctgacgagaagaaggctgaggaatttactagacagatcaaccgtgagaaggcaaagaccgctctgaatgcatatctcagaaatactaagtggaatgtgataatcagggaagatcttcttagaatcgataataagacatgtacgctctttagaaataaggccgttcatcttgaagttgcaagatatgttcatgcatatatcaacgatattgccgaagtaaacagctatttccagctttatcattacatcatgcagagaatcatcatgaacgaaagatatgaaaagtcttctggaaaggtaagcgaatacttcgatgctgtgaacgatgaaaagaagtacaacgacaggcttctgaagctgttgtgcgttccatttggttactgcatcccgagattcaagaatctctccattgaagctttgttcgacaggaacgaagcagctaagtttgacaaggaaaagaagaaagtatcaggtaattcatag

SEQ ID NO: 212 - protein, 967 aa, Ruminococcus flavefaciens
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 213 - DNA, 2769 bp, Artificial Sequence
  Ruminococcus_bicirculans
  CDS (1)..(2769)
atggcaaaaaagaataaaatgaagcctagagagctgcgtgaggctcagaaaaaagccagacagctcaaagcggctgagataaataataacgctgctcctgcgatcgctgccatgcctgctgcagaggtcattgcacctgtggcagagaagaaaaaatcctccgtaaaggcggcaggaatgaagtctattcttgtcagcgaaaataaaatgtacataacctctttcggcaagggcaattctgctgtgcttgaatatgaggtggacaataatgactacaacaaaactcagctttcttcaaaggacaacagcaatatcgagcttggtgatgtaaacgaggtaaacatcactttttcaagcaagcatggctttgggagcggagtggagataaatacttcaaaccctactcacagaagcggtgaaagctcgcctgtaagaggggatatgctggggcttaaatcggagcttgaaaagcgctttttcggcaaaacttttgatgataatatacatatccagcttatttacaacattctggatatcgaaaagatacttgcggtgtatgtaacgaatatcgtttatgcgctgaacaatatgcttggtataaaggattctgaaagttatgatgattttatggggtatctttctgcaagaaatacttatgaagtttttactcaccctgacaaaagcaatctttccgataaggtaaagggtaatatcaagaaaagccttagcaagtttaatgacttgctgaaaactaagcgccttggctatttcggccttgaagagccaaagacaaaagacacaagagcttcggaagcatacaaaaagcgtgtttatcatatgcttgcaattgtggggcagataagacagtgtgtttttcatgataaatcgggtgcaaaaagatttgacctttacagttttattaacaatattgatcccgaatacagagatactcttgactatcttgttgaggagcgtttaaagtccataaacaaggactttatcgagggtaacaaggtcaatatcagcctgcttattgatatgatgaaaggctatgaggctgatgatatcatacgcctttattacgatttcattgtgcttaaatctcagaaaaatctcggcttttctatcaaaaagcttcgtgagaaaatgctggaggaatacggtttcagatttaaggacaagcaatatgactctgtgcgctcaaagatgtacaagcttatggatttcctgcttttctgcaactactacagaaatgacgttgccgcaggcgaagctcttgtgcgtaaactgcgtttttcaatgaccgatgatgaaaaagaggggatatatgctgatgaagcggcaaagctttggggcaaattcaggaatgattttgaaaatatcgccgaccacatgaacggtgacgttatcaaggagcttggcaaggctgacatggattttgatgagaaaattcttgacagtgaaaagaagaatgcgtctgaccttttgtatttctccaaaatgatatatatgctcacatattttcttgacggcaaggagataaacgatcttcttacaacgcttatcagcaagtttgataacatcaaggagtttttgaagataatgaaaagctctgctgttgatgttgagtgtgagcttacggcgggctacaagctgttcaatgacagccagaggataaccaacgagctttttatcgtaaagaacattgcttccatgagaaagcctgcggcttcagcgaagcttacgatgttccgtgacgcactgactatactcggtatagacgacaatatcacggacgataggataagcgagattctaaaacttaaagaaaaaggcaagggcatacatggtctgagaaattttataacaaacaatgttatcgagtcctctcggtttgtataccttatcaagtatgcgaacgctcagaagataagagaagtggctaaggatgagaaagttgtcatgtttgttcttgggggtatccctgacacgcagatagagcgttattacaagagttgtgtggagtttcctgacatgaatagttctttggaagcaaagcgcagtgagcttgcgagaatgataaagaacatcagctttgatgatttcaaaaatgtgaaacagcaggcaaagggcagagaaaacgtggctaaggagagggcaaaggctgttatcgggctttatcttacggtcatgtatctgctggtgaaaaatcttgtgaatgtcaatgcaaggtatgttattgcgatacactgccttgaacgtgattttgggctgtataaggagataattcctgagttggcttcaaagaacttgaaaaatgactacaggatactttcacagacgctttgtgaactttgtgatgatcgtaatgagtcgtcgaatttgttcttgaaaaagaacaagcggctgcgcaagtgcgttgaagttgatatcaataatgcagacagcagcatgacaagaaaataccgcaactgtattgctcatcttactgtagttcgtgaactgaaagaatacataggagatattcgtacagtggattcttacttctccatttatcattatgttatgcagcgttgtatcacgaaaaggggagatgacacaaagcaagaagagaaaataaagtatgaggacgatcttttaaaaaatcacggctatacgaaagactttgtaaaggctctcaactcgccgtttggatacaacattccgaggtttaaaaatctttcaattgagcagttgtttgacagaaatgaatatcttactgaaaagtag

SEQ ID NO: 214 - protein, 922 aa, Artificial Sequence
  Synthetic Construct
MAKKNKMKPRELREAQKKARQLKAAEINNNAAPAIAAMPAAEVIAPVAEKKKSSVKAAGMKSILVSENKMYITSFGKGNSAVLEYEVDNNDYNKTQLSSKDNSNIELGDVNEVNITFSSKHGFGSGVEINTSNPTHRSGESSPVRGDMLGLKSELEKRFFGKTFDDNIHIQLIYNILDIEKILAVYVTNIVYALNNMLGIKDSESYDDFMGYLSARNTYEVFTHPDKSNLSDKVKGNIKKSLSKFNDLLKTKRLGYFGLEEPKTKDTRASEAYKKRVYHMLAIVGQIRQCVFHDKSGAKRFDLYSFINNIDPEYRDTLDYLVEERLKSINKDFIEGNKVNISLLIDMMKGYEADDIIRLYYDFIVLKSQKNLGFSIKKLREKMLEEYGFRFKDKQYDSVRSKMYKLMDFLLFCNYYRNDVAAGEALVRKLRFSMTDDEKEGIYADEAAKLWGKFRNDFENIADHMNGDVIKELGKADMDFDEKILDSEKKNASDLLYFSKMIYMLTYFLDGKEINDLLTTLISKFDNIKEFLKIMKSSAVDVECELTAGYKLFNDSQRITNELFIVKNIASMRKPAASAKLTMFRDALTILGIDDNITDDRISEILKLKEKGKGIHGLRNFITNNVIESSRFVYLIKYANAQKIREVAKDEKVVMFVLGGIPDTQIERYYKSCVEFPDMNSSLEAKRSELARMIKNISFDDFKNVKQQAKGRENVAKERAKAVIGLYLTVMYLLVKNLVNVNARYVIAIHCLERDFGLYKEIIPELASKNLKNDYRILSQTLCELCDDRNESSNLFLKKNKRLRKCVEVDINNADSSMTRKYRNCIAHLTVVRELKEYIGDIRTVDSYFSIYHYVMQRCITKRGDDTKQEEKIKYEDDLLKNHGYTKDFVKALNSPFGYNIPRFKNLSIEQLFDRNEYLTEK

SEQ ID NO: 215 - protein, 954 aa, Artificial Sequence
  Eubacterium siraeum with mutated HEPN domain
MGKKIHARDLREQRKTDRTEKFADQNKKREAERAVPKKDAAVSVKSVSSVSSKKDNVTKSMAKAAGVKSVFAVGNTVYMTSFGRGNDAVLEQKIVDTSHEPLNIDDPAYQLNVVTMNGYSVTGHRGETVSAVTDNPLRRFNGRKKDEPEQSVPTDMLCLKPTLEKKFFGKEFDDNIHIQLIYNILDIEKILAVYSTNAIYALNNMSADENIENSDFFMKRTTDETFDDFEKKKESTNSREKADFDAFEKFIGNYRLAYFADAFYVNKKNPKGKAKNVLREDKELYSVLTLIGKLAHWCVASEEGRAEFWLYKLDELKDDFKNVLDVVYNRPVEEINNRFIENNKVNIQILGSVYKNTDIAELVRSYYEFLITKKYKNMGFSIKKLRESMLEGKGYADKEYDSVRNKLYQMTDFILYTGYINEDSDRADDLVNTLRSSLKEDDKTTVYCKEADYLWKKYRESIREVADALDGDNIKKLSKSNIEIQEDKLRKCFISYADSVSEFTKLIYLLTRFLSGKEINDLVTTLINKFDNIRSFLEIMDELGLDRTFTAEYSFFEGSTKYLAELVELNSFVKSCSFDINAKRTMYRDALDILGIESDKTEEDIEKMIDNILQIDANGDKKLKKNNGLRNFIASNVIDSNRFKYLVRYGNPKKIRETAKCKPAVRFVLNEIPDAQIERYYEACCPKNTALCSANKRREKLADMIAEIKFENFSDAGNYQKANVTSRTSEAEIKRKNQAIIRLYLTVMYIMLKNLVNVNARYVIAFHCVERDTKLYAESGLEVGNIEKNKTNLTMAVMGVKLENGIIKTEFDKSFAENAANRYLRNARWYKLILDNLKKSERAVVNEFANTVCALNAIRNININIKEIKEVENYFALYHYLIQKHLENRFADKKVERDTGDFISKLEEHKTYCKDFVKAYCTPFGYNLVRYKNLTIDGLFDKNYPGKDDSDEQK

SEQ ID NO: 216 - protein, 919 aa, Artificial Sequence
  Uncultured Ruminococcus sp. with mutated HEPN site
MAKKNKMKPRELREAQKKARQLKAAEINNNAAPAIAAMPAAEVIAPAAEKKKSSVKAAGMKSILVSENKMYITSFGKGNSAVLEYEVDNNDYNQTQLSSKDNSNIQLGGVNEVNITFSSKHGFESGVEINTSNPTHRSGESSPVRGDMLGLKSELEKRFFGKTFDDNIHIQLIYNILDIEKILAVYVTNIVYALNNMLGVKGSESHDDFIGYLSTNNIYDVFIDPDNSSLSDDKKANVRKSLSKFNALLKTKRLGYFGLEEPKTKDNRVSQAYKKRVYHMLAIVGQIAQCVFADKSGAKRFDLYSFINNIDPEYRDTLDYLVEERLKSINKDFIEDNKVNISLLIDMMKGYEADDIIRLYYDFIVLKSQKNLGFSIKKLREKMLDEYGFRFKDKQYDSVRSKMYKLMDFLLFCNYYRNDIAAGESLVRKLRFSMTDDEKEGIYADEAAKLWGKFRNDFENIADHMNGDVIKELGKADMDFDEKILDSEKKNASDLLYFSKMIYMLTYFLDGKEINDLLTTLISKFDNIKEFLKIMKSSAVDVECELTAGYKLFNDSQRITNELFIVKNIASMRKPAASAKLTMFRDALTILGIDDKITDDRISGILKLKEKGKGIHGLRNFITNNVIESSRFVYLIKYANAQKIREVAKNEKVVMFVLGGIPDTQIERYYKSCVEFPDMNSSLGVKRSELARMIKNISFDDFKNVKQQAKGRENVAKERAKAVIGLYLTVMYLLVKNLVNVNARYVIAIHCLERDFGLYKEIIPELASKNLKNDYRILSQTLCELCDKSPNLFLKKNERLRKCVEVDINNADSSMTRKYANCIAALTVVRELKEYIGDICTVDSYFSIYHYVMQRCITKRENDTKQEEKIKYEDDLLKNHGYTKDFVKALNSPFGYNIPRFKNLSIEQLFDRNEYLTEK

SEQ ID NO: 217 - protein, 6 aa, Artificial Sequence
  native HEPN domain
  MISC_FEATURE (2)..(5): X is any amino acid
RXXXXH

SEQ ID NO: 218 - DNA, 33 bp, Artificial Sequence
  NLS signal
  CDS (1)..(33)
agccccaagaagaagagaaaggtggaggccagc

SEQ ID NO: 219 - protein, 11 aa, Artificial Sequence
  Synthetic Construct
SPKKKRKVEAS

SEQ ID NO: 220 - DNA, 33 bp, Artificial Sequence
  NLS signa
  CDS (1)..(33)
ggacctaagaaaaagaggaaggtggcggccgct

SEQ ID NO: 221 - protein, 11 aa, Artificial Sequence
  Synthetic Construct
GPKKKRKVAAA

SEQ ID NO: 222 - protein, 1020 aa, Artificial Sequence
  contig e-k87_11092736
MKRQKTFAKRIGIKSTVAYGQGKYAITTFGKGSKAEIAVRSADPPEETLPTESDATLSIHAKFAKAGRDGREFKCGDVDETRIHTSRSEYESLISNPAESPREDYLGLKGTLERKFFGDEYPKDNLRIQIIYSILDIQKILGLYVEDILHFVDGLQDEPEDLVGLGLGDEKMQKLLSKALPYMGFFGSTDVFKVTKKREERAAADEHNAKVFRALGAIRQKLAHFKWKESLAIFGANANMPIRFFQGATGGRQLWNDVIAPLWKKRIERVRKSFLSNSAKNLWVLYQVFKDDTDEKKKARARQYYHFSVLKEGKNLGFNLTKTREYFLDKFFPIFHSSAPDVKRKVDTFRSKFYAILDFIIYEASVSVANSGQMGKVAPWKGAIDNALVKLREAPDEEAKEKIYNVLAASIRNDSLFLRLKSACDKFGAEQNRPVFPNELRNNRDIRNVRSEWLEATQDVDAAAFVQLIAFLCNFLEGKEINELVTALIKKFEGIQALIDLLRNLEGVDSIRFENEFALFNDDKGNMAGRIARQLRLLASVGKMKPDMTDAKRVLYKSALEILGAPPDEVSDEWLAENILLDKSNNDYQKAKKTVNPFRNYIAKNVITSRSFYYLVRYAKPTAVRKLMSNPKIVRYVLKRLPEKQVASYYSAIWTQSESNSNEMVKLIEMIDRLTTEIAGFSFAVLKDKKDSIVSASRESRAVNLEVERLKKLTTLYMSIAYIAVKSLVKVNARYFIAYSALERDLYFFNEKYGEEFRLHFIPYELNGKTCQFEYLAILKYYLARDEETLKRKCEICEEIKVGCEKHKKNANPPYEYDQEWIDKKKALNSERKACERRLHFSTHWAQYATKRDENMAKHPQKWYDILASHYDELLALQATGWLATQARNDAEHLNPVNEFDVYIEDLRRYPEGTPKNKDYHIGSYFEIYHYIRQRAYLEEVLAKRKEYRDSGSFTDEQLDKLQKILDDIRARGSYDKNLLKLEYLPFAYNLPRYKNLTTEALFDDDSVSGKKRVAEWRER

SEQ ID NO: 223 - RNA, 36 nt, Ruminococcus flavefaciens
caaguaaaccccuaccaacuggucgggguuugaaac

SEQ ID NO: 224 - RNA, 36 nt, Artificial Sequence
  Ruminococcus_bicirculans
cuacuacacuggugcgaauuugcacuagucuaaaac

SEQ ID NO: 225 - DNA, 3965 bp, Artificial Sequence
  AAV2 transfer plasmid encoding N-terminal Abe8e with two sgRNA expression cassettes
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggaaaaaaagcaccgactcggtgccactttttcaagttgataacggactagccttattttaacttgctatttctagctctaaaacgctgcagaacaggagataaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcacctaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttacttctaggcgcgccaccatgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaattctgttggctgggccgtgatcaccgacgagtacaaggtgcccagcaagaaattcaaggtgctgggcaacaccgaccggcacagcatcaagaagaatctgatcggcgccctgctgttcgactctggcgaaacagccgaagccaccagactgaagaggacagccagacggcggtacaccagaagaaagaaccggatctgctacctgcaagagatcttcagcaacgagatggccaaggtggacgacagcttcttccaccggctggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgcccggctgagcaagagcagacggctggaaaacctgatcgctcagctgcccggcgagaagaagaatggcctgttcggcaacctgattgccctgagcctgggcctgacacctaacttcaagagcaacttcgacctggccgaggacgccaaactgcagctgtccaaggacacctacgacgacgacctggacaatctgctggcccagatcggcgatcagtacgccgacttgtttctggccgccaagaacctgtccgacgccatcctgctgagcgacatcctgagagtgaacaccgagatcacaaaggcccctctgagcgcctctatgatcaagagatacgacgagcaccaccaggatctgaccctgctgaaggccctcgttagacagcagctgcctgagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgcggaagcagcggaccttcgacaatggctctatccctcaccaaatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaggaagtggtggataagggcgccagcgctcagtccttcatcgagcggatgaccaacttcgataagaacctgcctaacgagaaggtaagtattagctctttctttccatgggttggcctcgccgcgtgggctgagggaaggactgtcctgggactggacaggcgggttatgggacctgaagcgataaaaggcatgcacgtttgcggctacgtgcatgccaaaaggagtcgggcttgcctccgtgcccgactccaaaagacctgctcgaggaggtggacgagcaggtcaaaaatccgggtaccaataaaatatctttattttcattacatctgtgtgttggttttttgtgtgactagttgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcgtataagaactgtatgagaccacggttatctcctgttctgcagcgttttagagctagaaatagcaagttaaaataaggctagtccgttatcaacttgaaaaagtggcaccgagtcggtgctttttttactagtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 226 - DNA, 4551 bp, Artificial Sequence
  AAV2 transfer plasmid encoding C-terminal Abe8e with two sgRNA expression cassettes
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggaaaaaaagcaccgactcggtgccactttttcaagttgataacggactagccttattttaacttgctatttctagctctaaaacgctgcagaacaggagataaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcacctaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagcttggatgttgcctttaggatttttgacctgctcgattgtccactgcgagcaggtcttttggagtcgggcgaggcggaagcccgactccttttggcatgcacgctagccgcgtcgtgcatgccttttatcttcgggttatgggaccagtgaaggctgagggaaggactgtcctgggactggacaggcgggttatgggacctgaaaatactaacaatcgattttttttccctttttttccaggtgctgcccaagcacagcctgctgtacgagtacttcaccgtgtacaacgagctgaccaaagtgaaatacgtgaccgagggaatgagaaagcccgcctttctgagcggcgagcagaaaaaggccattgtggatctgctgttcaagaccaaccggaaagtgaccgtgaagcagctgaaagaggactacttcaagaaaatcgagtgcttcgacagcgtggaaatcagcggcgtggaagatcggttcaatgccagcctgggcacataccacgacctgctgaaaattatcaaggacaaggacttcctggacaacgaagagaacgaggacatcctggaagatatcgtgctgaccctgacactgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaatcggaacttcatgcagctgatccacgacgacagcctgacctttaaagaggatatccagaaagcccaggtgtccggccagggcgattctctgcatgagcacattgccaacctggccggctctcccgccattaagaagggcattctgcagacagtgaaggtggtggacgagctggtcaaagtcatgggcagacacaagcccgagaacatcgtgatcgaaatggccagagagaaccagaccacacagaagggccagaagaacagccgcgagagaatgaagcggatcgaagagggcatcaaagagctgggcagccagatcctgaaagaacaccccgtggaaaacacccagctgcagaacgagaagctgtacctgtactacctccaaaacggccgggatatgtatgtggaccaagagctggacatcaaccggctgtccgactacgatgtggaccatatcgtgccccagtcttttctgaaagacgactccatcgacaacaaggtcctgaccagaagcgacaagaaccggggcaagagcgataacgtgccctccgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcaggcccaagaggaacagcgataagctgatcgccagaaagaaggactgggaccctaagaagtacggcggcttcgtcagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccagattcctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctagggccttcaagtactttgacaccaccatcgaccggaaggtgtacaggagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaaggtaccaattcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaactagttgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcgtataagaactgtatgagaccacggttatctcctgttctgcagcgttttagagctagaaatagcaagttaaaataaggctagtccgttatcaacttgaaaaagtggcaccgagtcggtgctttttttactagtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

(19) United States
(12) Patent Application Publication
(10) Pub. No.: US 2024/0216544 A1
(43) Pub. Date: Jul. 4, 2024

(54) METHODS AND COMPOSITIONS FOR EXPRESSION OF EDITING PROTEINS

(71) Applicant: Salk Institute for Biological Studies, La Jolla, CA (US)

(72) Inventors: Lukas Christoph Bachmann, San Diego, CA (US); Samuel Lawrence Pfaff, San Diego, CA (US)

(73) Assignee: Salk Institute for Biological Studies, La Jolla, CA (US)

(21) Appl. No.: 18/289,166

(22) PCT Filed: May 16, 2022
(86) PCT No.: PCT/US2022/029459

§ 371 Date: Nov. 1, 2023

Related U.S. Application Data
(60) Provisional application No. 63/189,048, filed on May 14, 2021.

(51) Int. Cl. A61K 48/00; A61K 31/7105; A61K 38/46; C12N 9/22; C12N 15/11

(52) U.S. Cl. CPC A61K 48/0066; A61K 31/7105; A61K 38/465; A61K 48/0033; A61K 48/0058; C12N 9/22; C12N 15/11; C12N 2310/20; C12N 2800/80

(57) ABSTRACT

Provided herein are compositions and systems for reconstitution of RNA molecules, including methods for using these molecules. For example, such molecules can be used to deliver a protein coding sequence over two or more viral vectors (such as AAVs), resulting in reconstitution of the full-length protein in a cell. Such methods can be used to deliver a protein involved in editing a nucleic acid molecule, for example to treat a genetic disease or cancer.

### CROSS-REFERENCE TO RELATED APPLICATION

This application claims priority to U.S. Provisional Application No. 63/189,048 filed May 14, 2021, herein incorporated by reference in its entirety.

### FIELD

The present disclosure provides systems, kits, compositions, and methods that allow for joining of two or more RNA molecules, allowing expression of a full-length protein, such as a protein involved in gene editing such as a Cas nuclease, or catalytically inactive forms of a Cas nuclease.

### BACKGROUND

Gene therapy is a promising method for treating genetic diseases caused by loss-of-function mutations. Replacement genes are typically reintroduced into target cells using vectors such as AAV because the virus is generally safe and efficient at entering cells. However, in the case of AAV it is difficult to encapsulate more than about 5000 nucleotides using conventional capsids. Since the length of genes that encode large proteins often exceed the packaging constraints of AAV, many genetic diseases remain untreatable. Strategies to overcome this limitation have been explored in the past, but proved inefficient, led to expression of high levels of potentially toxic truncated protein, or both. Safe, high efficiency strategies for delivery of large proteins to treat disease are needed.

### SUMMARY

Provided herein are compositions for expressing a target protein, such as a protein used to edit a nucleic acid sequence (such as target DNA or RNA, such as a gene). Included are compositions and methods for expressing a nucleic acid editing protein produced from two or more synthetic nucleic acid molecules introduced individually to the same cell. Using this strategy, a full-length nucleic acid editing protein and one or more guide RNAs can be provided to the same cell, resulting in targeted nucleic acid editing. The cell may be in need of targeted nucleic acid editing to repair a mutation, e.g., in an essential gene. In one example, the composition includes (a) a first RNA molecule, the first RNA molecule comprising from 5′ to 3′: (i) a coding sequence for an N-terminal portion of the nucleic acid editing protein; (ii) a splice donor; and (iii) a first dimerization domain; and (b) a second RNA molecule, the second RNA molecule comprising from 5′ to 3′: (i) a second dimerization domain, wherein the second dimerization domain binds to the first dimerization domain; (ii) a branch point sequence; (iii) a polypyrimidine tract; (iv) a splice acceptor; and (v) a coding sequence for a C-terminal portion of the nucleic acid editing protein. In some examples, such a composition further includes one or more of (c) a third RNA molecule comprising at least one first guide RNA (gRNA) specific for a first target nucleic acid molecule, wherein the at least one first gRNA directs the nucleic acid editing protein to a target editing site on the first target nucleic acid molecule; (d) a fourth RNA molecule comprising at least one second gRNA specific for (i) the first target nucleic acid molecule, wherein the at least one second gRNA directs the nucleic acid editing protein to the same or different target editing site on the first nucleic acid molecule, or (ii) a second target nucleic acid molecule, wherein the at least one second gRNA directs the nucleic acid editing protein to a target editing site on the second target nucleic acid molecule; (e) a fifth RNA molecule comprising at least one third gRNA specific for (i) the first target nucleic acid molecule, wherein the at least one third gRNA directs the nucleic acid editing protein to the same or different target editing site on the first nucleic acid molecule as the first and second gRNA, (ii) the second target nucleic acid molecule, wherein the at least one third gRNA directs the nucleic acid editing protein to the same or different target editing site on the second nucleic acid molecule as the second gRNA, or (iii) a third target nucleic acid molecule, wherein the at least one third gRNA directs the nucleic acid editing protein to a target editing site on the third target nucleic acid molecule; and/or (f), a sixth RNA molecule comprising at least one fourth gRNA specific for (i) the first target nucleic acid molecule, wherein the at least one fourth gRNA directs the nucleic acid editing protein to the same or different target editing site on the first nucleic acid molecule as the first, second, and third gRNA, (ii) the second target nucleic acid molecule, wherein the at least one fourth gRNA directs the nucleic acid editing protein to the same or different target editing site on the second nucleic acid molecule as the second and third gRNA, (iii) the third target nucleic acid molecule, wherein the at least one fourth gRNA directs the nucleic acid editing protein to the same or different target editing site on the third target nucleic acid molecule as the third gRNA or (iv) a fourth target nucleic acid molecule, wherein the at least one fourth gRNA directs the nucleic acid editing protein to a target editing site on the fourth target nucleic acid molecule.

In one example, the composition includes (a) a first RNA molecule, the RNA molecule comprising from 5′ to 3′: (i) a coding sequence for an N-terminal portion of the nucleic acid editing protein; (ii) a splice donor, and (iii) a first dimerization domain; and (b) a second RNA molecule, the RNA molecule comprising from 5′ to 3′: (i) a second dimerization domain, wherein the second dimerization domain binds to the first dimerization domain; (ii) a branch point sequence; (iii) a polypyrimidine tract; (iv) a splice acceptor, and (v) a coding sequence for a C-terminal portion of the nucleic acid editing protein. In some examples, such a composition further includes one or more of (c) third and fourth RNA molecules comprising at least one first crisprRNA (crRNA) specific for a first target nucleic acid molecule, and at least one first tracrRNA, respectively, wherein the at least one first crRNA and at least one first tracrRNA direct the nucleic acid editing protein to a target editing site on the first target nucleic acid molecule; (d) fifth and sixth RNA molecules comprising at least one second crRNA specific for (i) the first target nucleic acid molecule, and at least one second tracrRNA, respectively, wherein the at least one second crRNA and at least one second tracrRNA direct the nucleic acid editing protein to the same or different target editing site on the first nucleic acid molecule as the first crRNA and the first tracrRNA, and the second crRNA and the second tracrRNA, or (ii) a second target nucleic acid molecule, wherein the at least one second crRNA and at least one second tracrRNA direct the nucleic acid editing protein to a target editing site on the second target nucleic acid molecule; (e) seventh and eighth RNA molecules comprising at least one third cRNA specific for (i) the first target nucleic acid molecule, and at least one third tracrRNA, respectively, wherein the at least one third crRNA and the at least one third tracrRNA direct the nucleic acid editing protein to the same or different target editing site on the first nucleic acid molecule as the first crRNA and the first tracrRNA, and the second crRNA and the second tracrRNA, (ii) the second target nucleic acid molecule, wherein the at least one third crRNA and the at least one third tracrRNA direct the nucleic acid editing protein to the same or different target editing site on the second nucleic acid molecule as the second crRNA and the second tracrRNA, or (iii) a third target nucleic acid molecule, wherein the at least one third crRNA and the at least one third tracrRNA direct the nucleic acid editing protein to a target editing site on the third target nucleic acid molecule; and/or (f), ninth and tenth RNA molecules comprising at least one fourth crRNA specific for (i) the first target nucleic acid molecule, and at least one fourth tracrRNA, respectively, wherein the at least one fourth crRNA and the at least one fourth tracrRNA direct the nucleic acid editing protein to the same or different target editing site on the first nucleic acid molecule as the first crRNA and the first tracrRNA, the second crRNA and the second tracrRNA, and the third crRNA and the third tracrRNA, (ii) the second target nucleic acid molecule, wherein the at least one fourth crRNA and the at least one fourth tracrRNA direct the nucleic acid editing protein to the same or different target editing site on the second nucleic acid molecule as the second crRNA and the second tracrRNA, and the third crRNA and the third tracrRNA, (iii) the third target nucleic acid molecule, wherein the at least one fourth crRNA and the at least one fourth tracrRNA direct the nucleic acid editing protein to the same or different target editing site on the third target nucleic acid molecule as the third crRNA and the third tracrRNA, or (iv) a fourth target nucleic acid molecule, wherein the at least one fourth crRNA and the at least one fourth tracrRNA direct the nucleic acid editing protein to a target editing site on the fourth target nucleic acid molecule.

In some examples, the first and second dimerization domains bind by direct binding, indirect binding, or both.

In some examples, the dimerization domains are kissing loop domains or hypodiverse domains.

In some examples, the first and/or second RNA molecule comprise at least one splice enhancer.

Also provided are compositions for expressing a target protein. Such compositions can include (i) a first synthetic DNA molecule encoding the RNA molecules of (a) and (c) or (a), (c) and (d); and (ii) a second synthetic DNA molecule encoding the RNA molecules of (b) and (e) or (b), (e) and (f). In some examples, the first synthetic DNA molecule comprises (i) a first promoter operably linked to a sequence encoding the first RNA molecule; and (b) a second promoter operably linked to a sequence encoding the second RNA molecule.

Also provided are systems for expressing a nucleic acid editing protein comprising the described compositions.

Also provided are methods of using the disclosed systems or the RNAs encoded by the systems to express a nucleic acid editing protein in a cell, for example in combination with appropriate guide nucleic acid molecules that hybridize to a target nucleic acid molecule. Such a method can include introducing the system into a cell, and expressing the synthetic first and second RNA molecules in the same cell. In some examples, the cell is in a subject, and the method treats a disease in the subject such as a genetic disease caused by a mutation in a target DNA or RNA (e.g., gene). In some examples the genetic disease is Duchenne Muscular Dystrophy, Hemophilia A, Stargardt's Disease, or Usher Syndrome.

The foregoing and other objects and features of the disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts a schematic of vector designs (left) and RNA interactions and splicing (right). Left: 5′ trans-splice (trsp) DNA vector: Open arrows are two opposing promoters. RFP coding domain and 3′UTR with poly adenylation elements are expressed opposite from the N-terminal portion of YFP (n-yfp), followed by a splice donor sequence (SD), a downstream intronic splicing enhancer (DISE), and two intronic splicing enhancers (2×ISE), a binding domain (BD, also referred to as dimerization domain), and a stable stem loop BoxB element (boxB), a self-cleaving hammerhead ribozyme (HHrz), ending with a 3′ UTR containing poly adenylation elements. The n-yfp segment has a small intron inserted (white segment within n-yfp). 3′ trsp DNA vector: Open arrows are two opposing promoters. BFP coding domain and 3′UTR with poly adenylation elements are expressed opposite from complementary binding domain (anti-BD, also referred to as dimerization domain), followed by three intronic splicing enhancer sequences (3×ISE), a branch point (BP), a polypyrimidine tract (PPT), a splice acceptor sequence (SA), the c-terminal proton of the YFP coding sequence, ending with a 3′ UTR containing poly adenylation elements. Right: pre-mRNA interactions (5′ trsp-RNA+3′ trsp-RNA) and trans-splicing to generate an mRNA encoding YFP protein are shown.

FIG. 1B depicts transfection of only the N-terminal expression plasmid does not lead to YFP fluorescence.

FIG. 1C depicts transfection of only the C-terminal expression plasmid does not lead to YFP fluorescence.

FIG. 1D depicts expression of N-terminal and C-terminal fragments without binding domains shows low levels of YFP induction.

FIG. 1E depicts rationally designed dimerization/binding domain in a looped configuration (hypodiverse sequence consisting of either all pyrimidines or all purines that are interrupted by complementary sequences that form double stranded stem structures).

FIG. 1F depicts 3D rendering of the “looped” dimerization domain configuration.

FIG. 1G depicts negative control with no binding domain on the C-terminal half.

FIG. 1H depicts negative control with no binding domain on the N-terminal half.

FIG. 1I depicts matching binding domains in a looped configuration on both N- and C-terminal half shows strong YFP induction in 90% of the cells.

FIGS. 1J-1N depict data equivalent to that in FIGS. 1E-II for a configuration of a binding domain with a 150 nucleotide hypodiverse sequence comprised exclusively of pyrimidine (or alternatively exclusively purine) containing sequence resulting in a fully open configuration.

FIG. 1J depicts a 150 nucleotide hypodiverse pyrimidine sequence resulting in a fully open configuration for complimentary base pairing.

FIG. 1K depicts a 3D rendering of the 150 nucleotide hypodiverse pyrimidine sequence from (1J).

FIG. 1L depicts a control HEK293T cell transfection with the C-terminal-YFP encoding construct lacking a complimentary hypodiverse binding domain. Few transfected cells express YFP.

FIG. 1M depicts a control HEK293T cell transfection with the N-terminal-YFP encoding construct lacking a complimentary hypodiverse binding domain. Few transfected cells express YFP.

FIG. 1N depicts a HEK293T cell transfection with N-terminal-YFP and C-terminal-YFP constructs that both have complimentary hypodiverse dimerization binding domains. Many cells express YFP at high levels.

FIG. 1O depicts representative fluorescence images for cells shown in FIG. 1G. The positive markers for transfection (RFP+BFP) are expressed, but YFP protein is not reconstituted efficiently.

FIG. 1P depicts representative fluorescence images for cells shown in FIG. 1L. The positive markers for transfection (RFP+BFP) are expressed, and YFP protein is reconstituted at high levels in cells that are both RFP and BFP double positive.

FIG. 1Q depicts a comparison of conditions shown in FIG. 1D, FIGS. 1G-1I, and FIGS. 1L-1N. N: no binding domain, Loop: looped hypodiverse binding domain configuration, Lin: linear hypodiverse configuration.

FIG. 2A depicts schematic of vector designs. The protein coding sequence of a yellow fluorescent protein (YFP) is split into an N-terminal, a middle fragment (m-yfp) and a C-terminal fragment. The junction of RNAs encoding the n and m fragments is joined by a looped design binding domain (BD1) and the junction between m and c fragments is joined by a looped binding domain (BD2). The pyrimidine (Y) and purine (R) sequences are arranged in such a way as to avoid self-circularization of the m-fragment and avoid direct recombination of the N- and C-fragment. The N-terminal fragment is co-expressed with red fluorescent protein as a transfection control, the C-terminal fragment is coexpressed with blue fluorescent protein as a transfection control. Promoter sequences are indicated with open arrows. Splice donor (SD) and splice acceptor (SA) sites are indicated. Intronic splicing elements including splice enhancers, polypyrimidine tracts and branch points are included, analogous to the elements used upstream (5′) of the SA and downstream (3′) of the SD in FIG. 1A.

FIG. 2B depicts human cell line transfection of plasmids I+II+III (see FIG. 2A) efficiently reconstituting high level YFP expression in 80% of the transfected cells.

FIG. 2C depicts representative fluorescent image of expression of the n and m fragment (plasmid I+II, see FIG. 2A) shows no yfp fluorescence (negative control).

FIG. 2D depicts representative fluorescent image of expression of the m and c fragment (plasmid I+mI, see FIG. 2A) shows no yfp fluorescence (negative control).

FIG. 2E depicts representative fluorescent image showing that strong YFP fluorescence is induced by co-transfection of all three fragments (plasmid I+II+III, see FIG. 2A).

FIGS. 3A-3D depict efficient reconstitution of yellow fluorescent protein (YFP) from two fragments (SEQ ID NOS: 1 and 2) expressed from two AAV2/8s after systemic administration in the newborn (P3) mouse pup. (A) depicts AAV 1 encoding the n-terminal half fragment of YFP, and AAV 2 encoding the c-terminal half fragment. AAV 1+AAV 2 were mixed at equal titer and injected intravenously into mice. Tissue sample were collected 3 weeks following injection. (B) depicts YFP fluorescence in the liver of the juvenile mouse at the time of sacrifice (green). Uninjected liver is shown for comparison (control: no YFP detected). DRAQ5 nuclear stain is shown in magenta for context. (C) depicts strong YFP fluorescence in the heart muscle at the time of sacrifice (green). Top panels show macroscopic view and red autofluorescence for context (in magenta). Bottom panel shows cross-section with DRAQ5 nuclear stain for context (in magenta). Uninjected mouse heart lacking YFP is shown for control. (D) depicts strong YFP fluorescence in the skeletal muscles of the leg at the time of sacrifice. Uninjected mouse legs are shown for comparison (negative control, no YFP detected). Top panels show macroscopic view with red autofluorescence in magenta. Bottom panel shows microscopic image of a cross-section through the leg. Bottom panel shows DRAQ5 nuclear stain in magenta for context.

FIGS. 4A-4B depict efficient reconstitution of yellow fluorescent protein (YFP) from three fragments (SEQ ID NOS: 145, 146 and 2, respectively) in the mouse tibialis anterior muscle after intramuscular injection of three AAV2/8 in the newborn (P3) mouse pup. (A) depicts a schematic of three AAV particles with separate N-, M-, and C-terminal fragments of YFP (analogous to FIG. 2A). (B) Shows strong YFP fluorescence in a longitudinal section of the tibialis anterior muscle of a mouse injected with all three viral particles. DRAQ5 nuclear stain is shown in magenta for context.

FIGS. 5A-5F depict efficient reconstitution of yellow fluorescent protein (YFP) from two and from three fragments in adult mouse tibialis anterior muscle. (A) depicts N-terminal and C-terminal halves of YFP coding sequence are equipped with synthetic RNA-dimerization and recombination domains. (B) depicts two AAV transfer plasmids expressing these two fragments were electroporated transcutaneously into adult mouse tibialis anterior (TA) muscle and strong fluorescence was detected at 5 days post electroporation. (C) depicts no fluorescence was detectable in contralateral non-injected TA. (D) depicts n-terminal, middle, and c-terminal YFP coding sequence are equipped with synthetic RNA-dimerization and recombination domains linking each fragment to its adjacent fragment(s). (E) depicts transcutaneous electroporation of three AAV transfer plasmids expressing these three fragments. Strong YFP fluorescence is detected indicating efficient reconstitution of YFP from three fragments. (F) depicts fluorescence in contralateral non-injected TA. Fluorescent channel is overlaid onto grey scale photographs for context.

FIG. 6A is a schematic drawing providing an exemplary system for the disclosed RNA recombination methods, using two nucleic acid molecules 110, 150, wherein the target protein is divided into two portions and each portion is encoded by a different nucleic acid molecule. In some examples, the nucleic acid molecules 110, 150, of the system are DNA, and include promoters 112, 152. In some examples, the nucleic acid molecules 110, 150, of the system are RNA, and thus lack the promoters 112, 152. Drawing not to scale.

FIG. 6B is a schematic drawing providing an exemplary dimerization domain (e.g., 122, 154 of FIG. 6A) that includes hypodiverse sequences interspersed with sequences that can form a stem, which results in local RNA loops that are open and available for basepairing in the absence of pseudoknot formation. Drawing not to scale.

FIG. 6C is a schematic drawing showing the interaction and hybridization (base pairing) between a pre-mRNA dimerization domain 122 of molecule 110 (FIG. 6A) and a pre-mRNA dimerization domain 154 of molecule 150 (FIG. 6A) allows the spliceosome components to recombine N-terminal coding sequence 114 and C-terminal coding sequence 164. This results in the 3′ end of the N-terminal protein coding sequence 114 fusing to the 5′ end of the C terminal protein sequence 164, and a seamless junction between the N- and C-terminal portions. Drawing not to scale.

FIG. 6D is a schematic drawing providing an exemplary system for the disclosed RNA recombination methods, using three nucleic acid molecules 110, 200, 150, wherein the target protein is divided into three portions (N-terminal, middle, C-terminal) and each portion is encoded by a different nucleic acid molecule. Prior to transcription, nucleic acid molecules 110, 150, 200 of the system are DNA, and include promoters 112, 152, 202. Following transcription, nucleic acid molecules 110, 150, 200 of the system are RNA, and thus lack the promoters 112, 152, 202. Drawing not to scale.

FIG. 6E is a schematic drawing showing the interaction and hybridization (base pairing) between dimerization domain 122 of molecule 110 (FIG. 6D) and dimerization domain 204 of molecule 200 (FIG. 6D), and between dimerization domain 226 of molecule 200 (FIG. 6D) and dimerization domain 154 of molecule 150 (FIG. 6D), allows the spliceosome components to recombine N-terminal coding sequence 114, middle coding sequence 216, and C-terminal coding sequence 164. This results in the 3′ end of the N terminal coding sequence 114 fusing to the 5′ end of the middle protein sequence 216, and the 3′ end of the middle coding sequence 216 fusing to the 5′ end of the C-terminal sequence 216, and a seamless junction between the N-, middle, and C-terminal portions. In some examples, for example following transcription, the elements shown are RNA. Drawing not to scale.

FIG. 6F is a schematic drawing providing an exemplary system for the disclosed RNA recombination methods, using two nucleic acid molecules 110, 150, wherein the target protein is divided into two portions and each portion is encoded by a different nucleic acid molecule. In this example, the DNA has been transcribed into RNA, such that nucleic acid molecules 110, 150, of the system are RNA, and thus lack the promoters 112, 152 present in the DNA (see FIG. 6A). Drawing not to scale.

FIG. 6G is a schematic drawing providing an exemplary composition or system for the disclosed RNA recombination methods, using two DNA molecules 110, 150, wherein the nucleic acid editing protein is divided into two portions 114, 164 and each portion is encoded by a different DNA molecule 110, 150 respectively. In some examples, promoters 112, 152 drive expression of each coding sequence 114, 164.

The system in this example includes one or more guide nucleic acid molecules (e.g., gRNA or gRNA coding sequence) 140, 141, 171, 172 which are specific for one or more target nucleic acid molecules. However, such guide nucleic acid molecules 140, 141, 171, 172 are optional, and in some examples instead of being provided as part of molecules 110, 150, are provided separately, for example as part of a separate vector. This example shows optional guide nucleic acid molecules 140, 141, 171, 172 near the 5′ and 3′-ends of nucleic acid molecules 110, 150, but the disclosure is not limited to such locations. If present, expression of one or more guide nucleic acid molecules 140, 141, 171, 172 can be driven by promoters 142, 143, 173, 174. The system can optionally include a parvovirus inverted terminal repeat (ITR) 176, 177, 178, 179 at each 5′ and 3′-end of molecules 110, 150. Although this figure shows an embodiment where molecules 110, 150 are DNA, in some examples (for example following transcription), the nucleic acid molecules 110, 150 of the system are RNA, and thus lack the guide nucleic acid molecules 140, 141, 171, 172, promoters 112, 152, 142, 143, 173, 174 and parvovirus ITR 176, 177, 178, 179. Drawing not to scale.

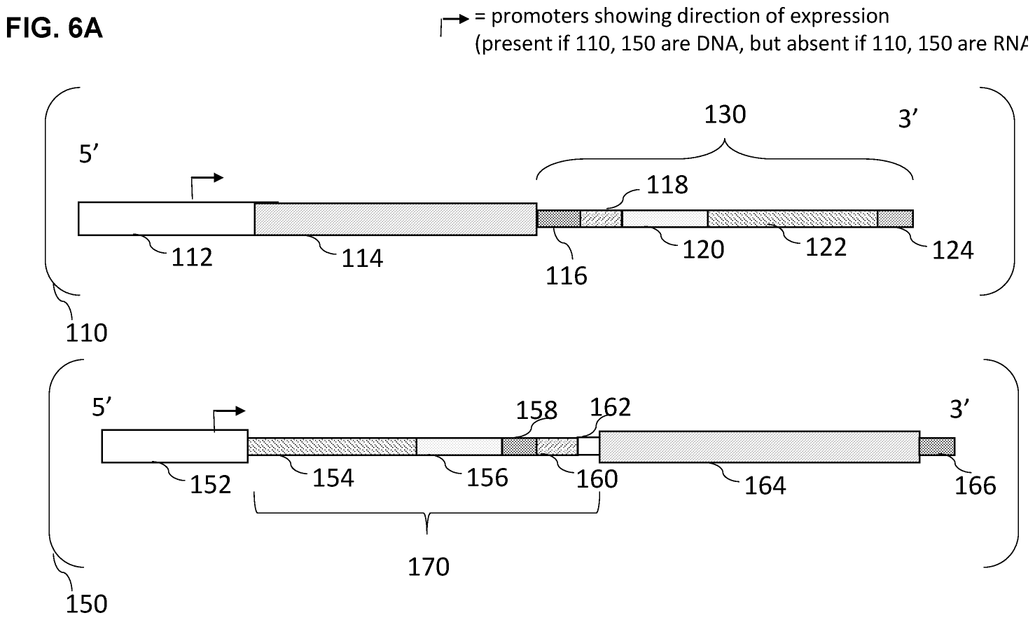

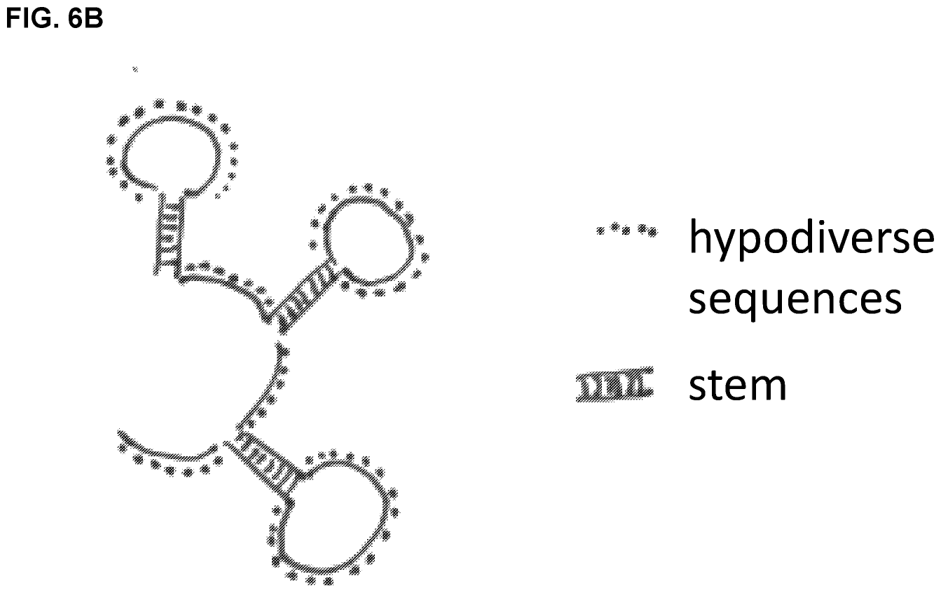

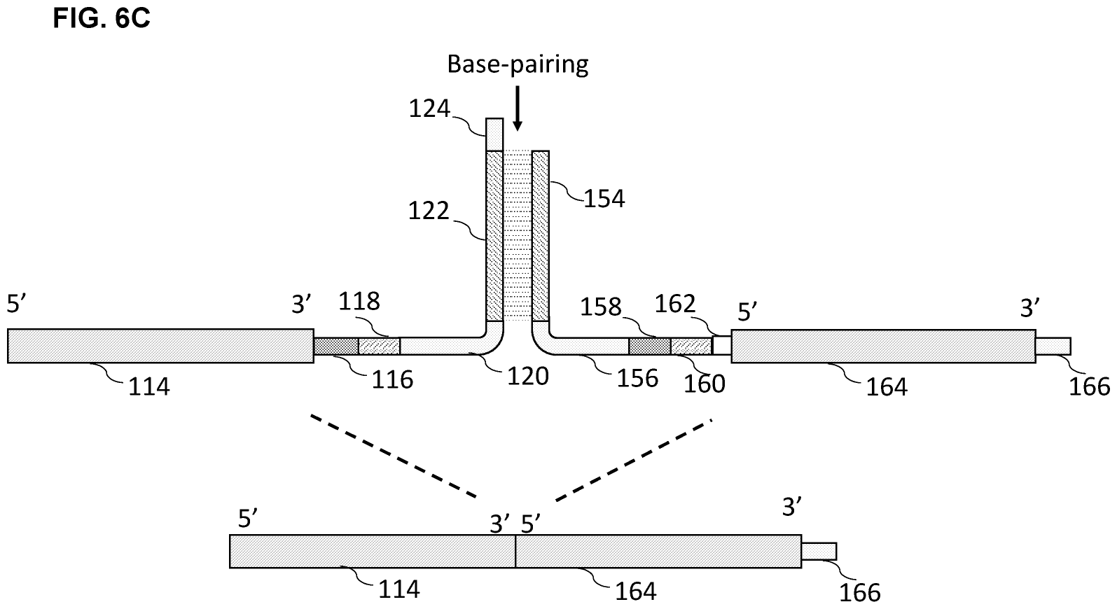

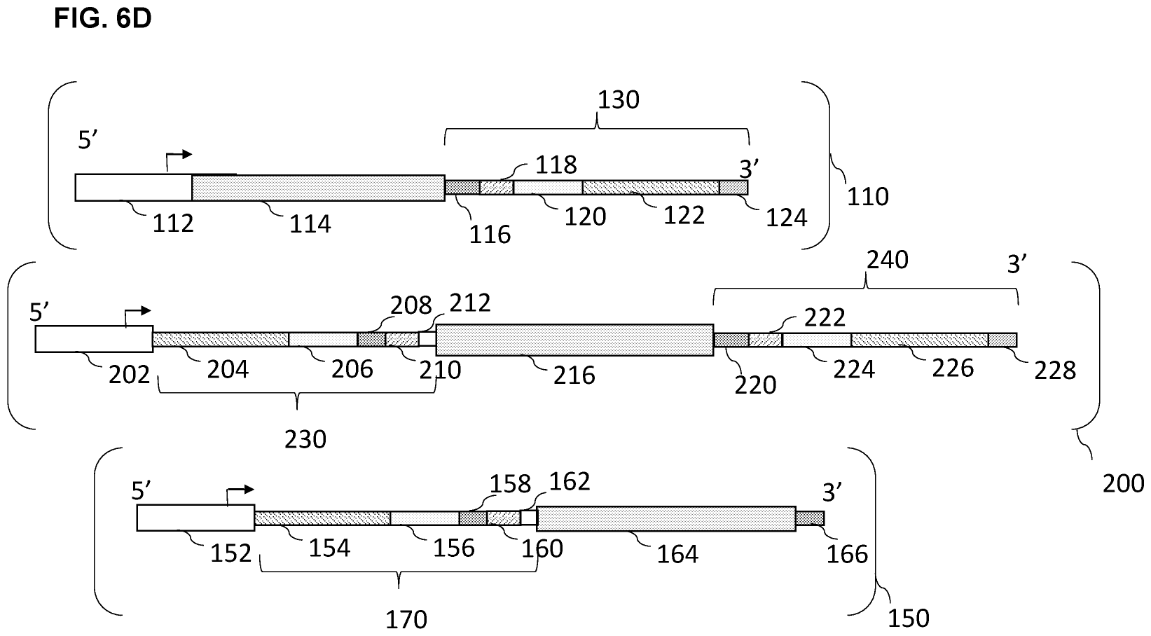

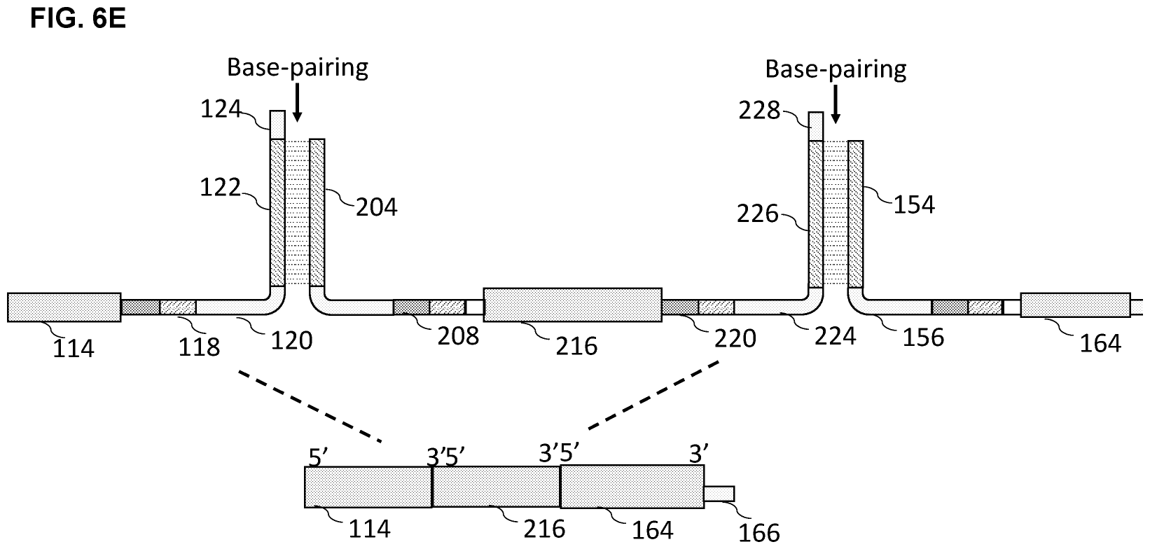

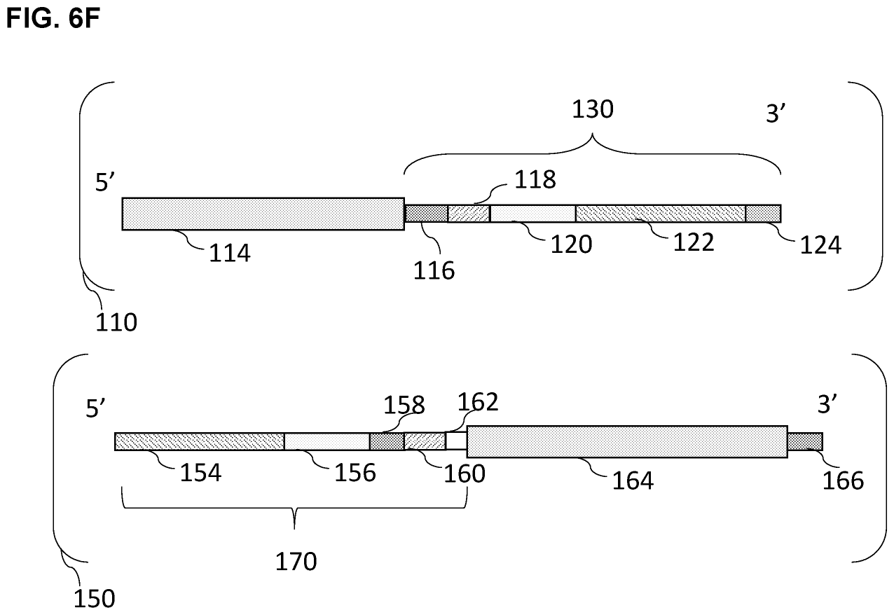

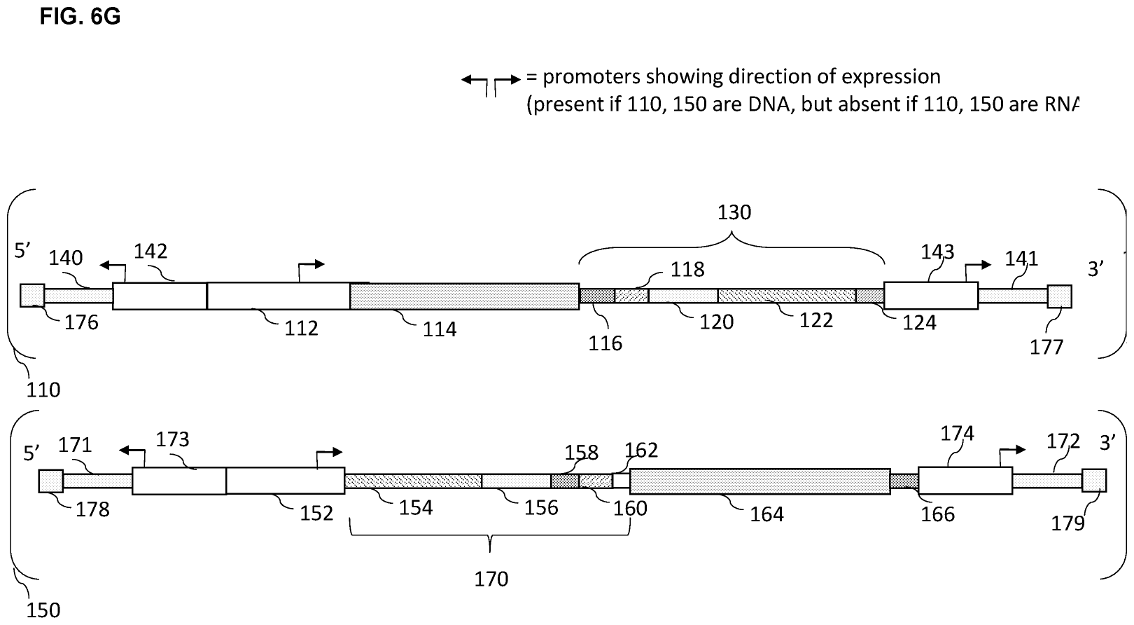

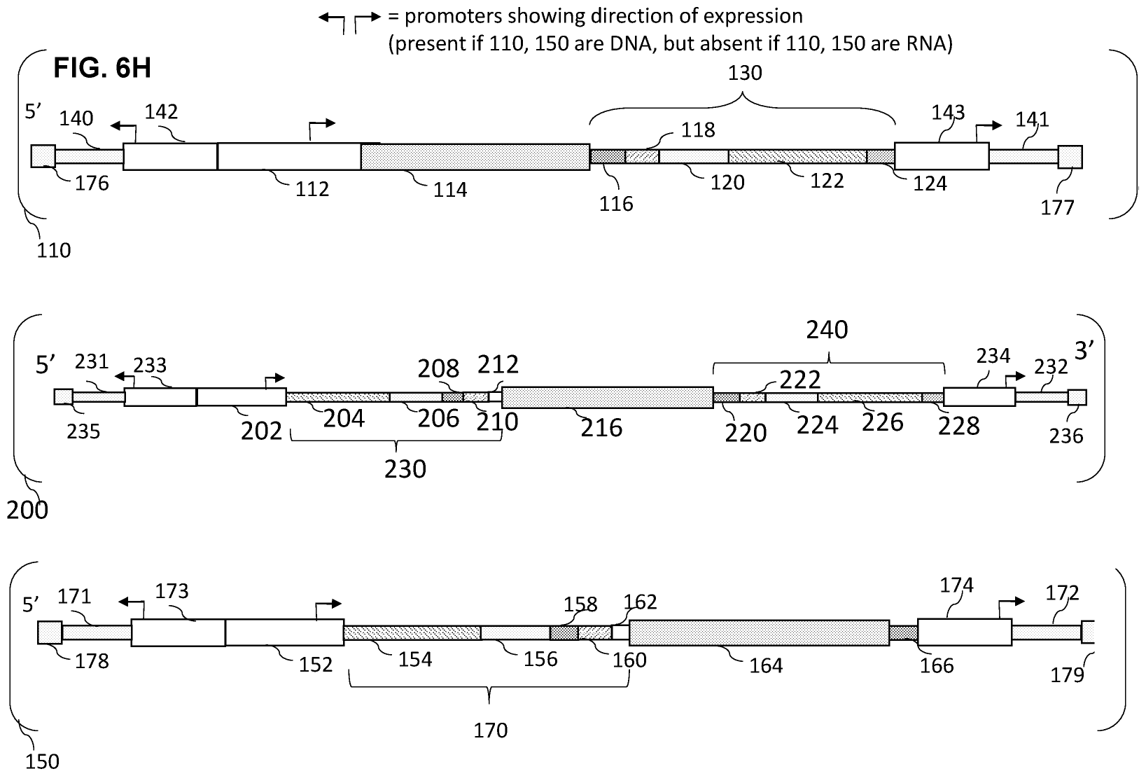

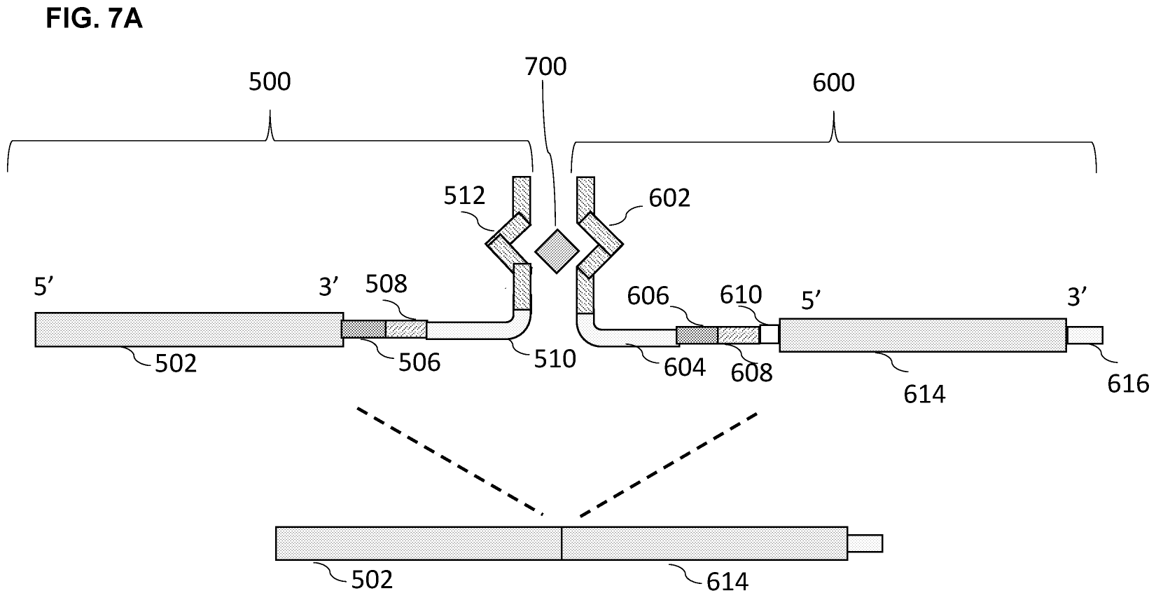

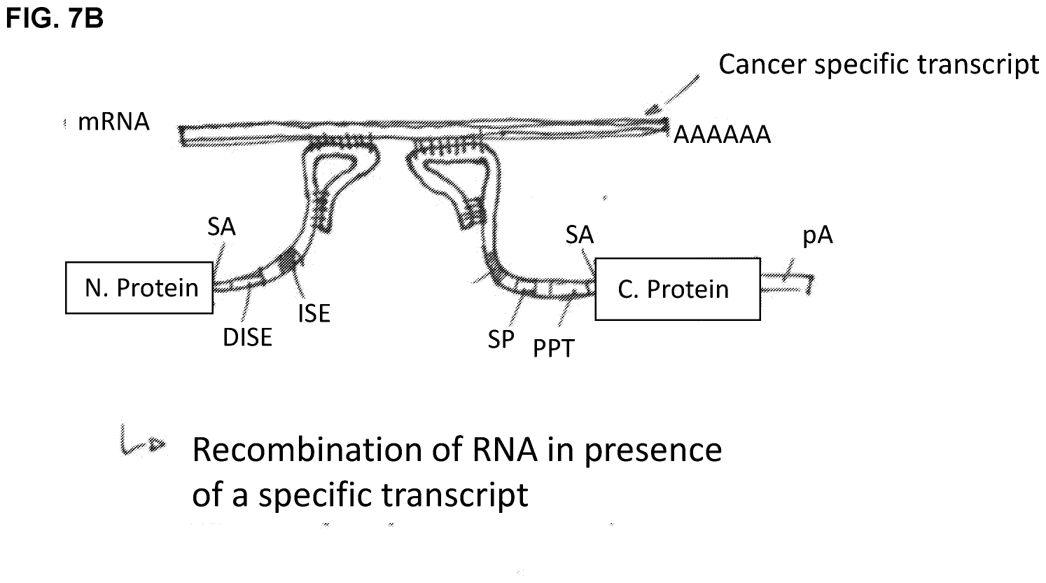

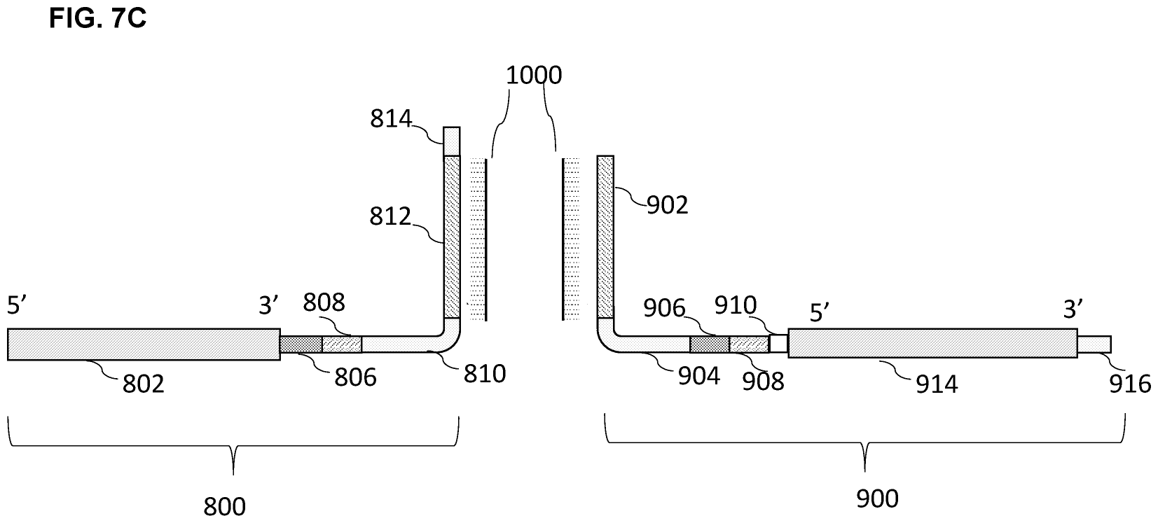

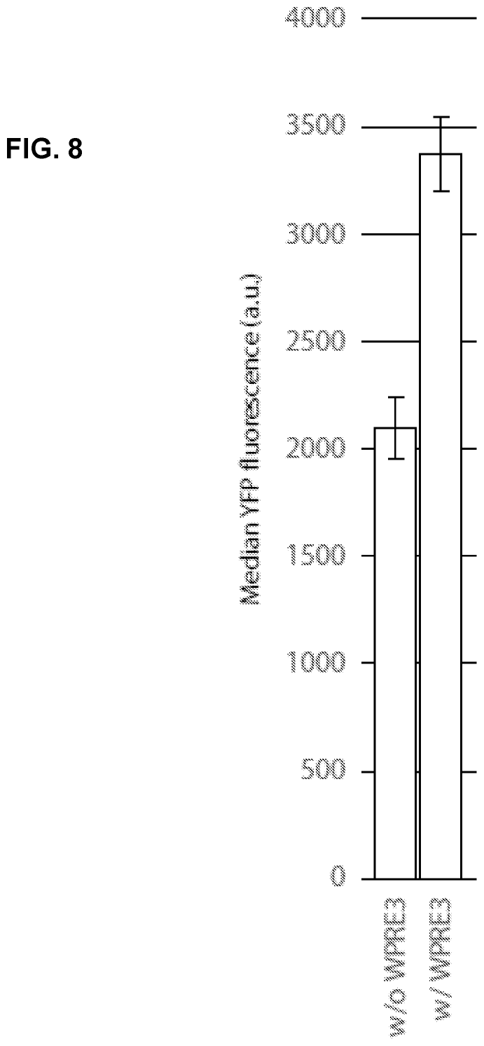

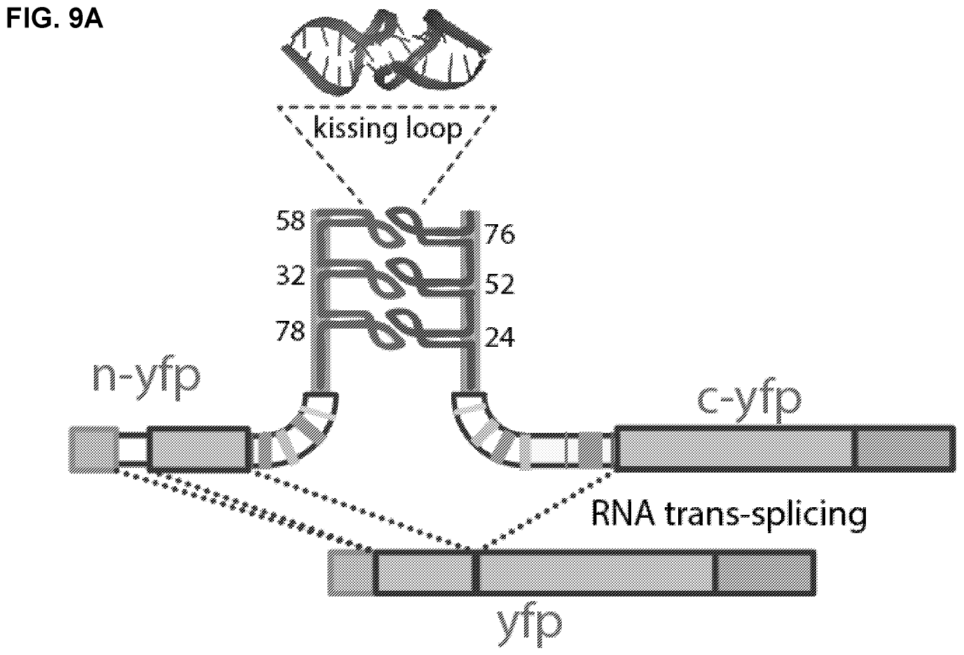

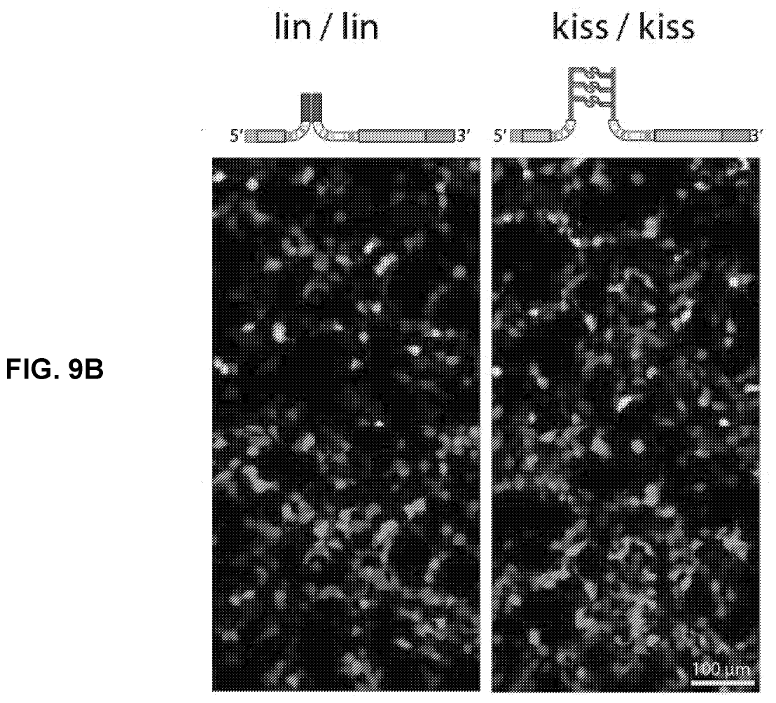

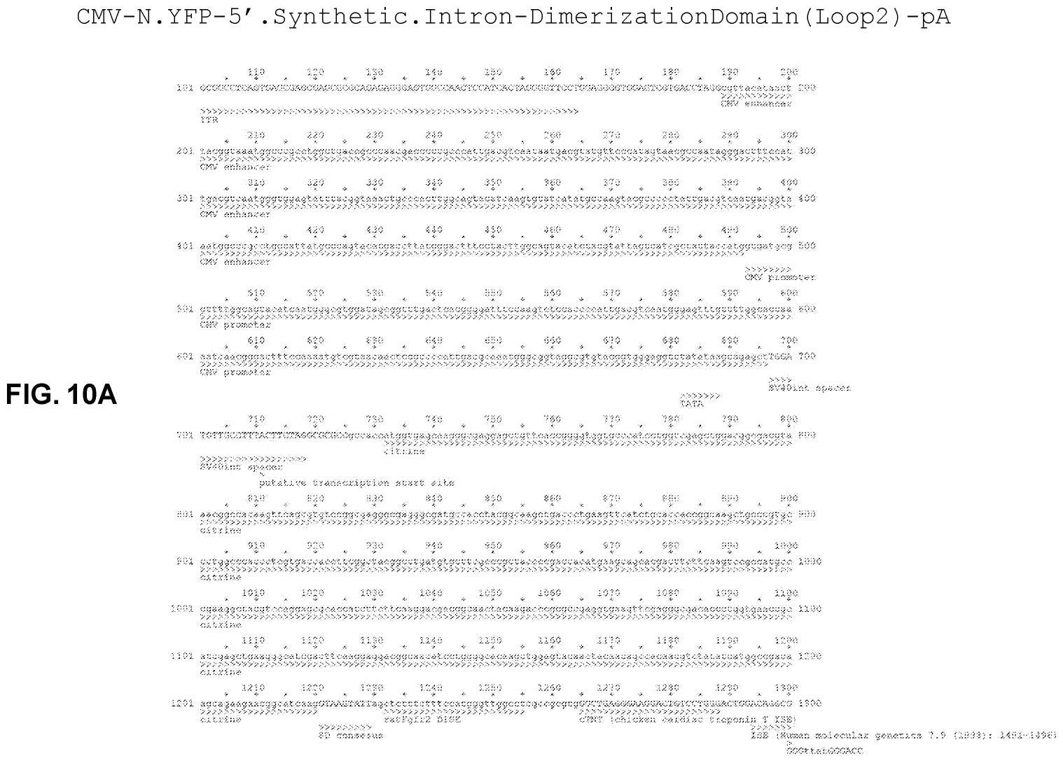

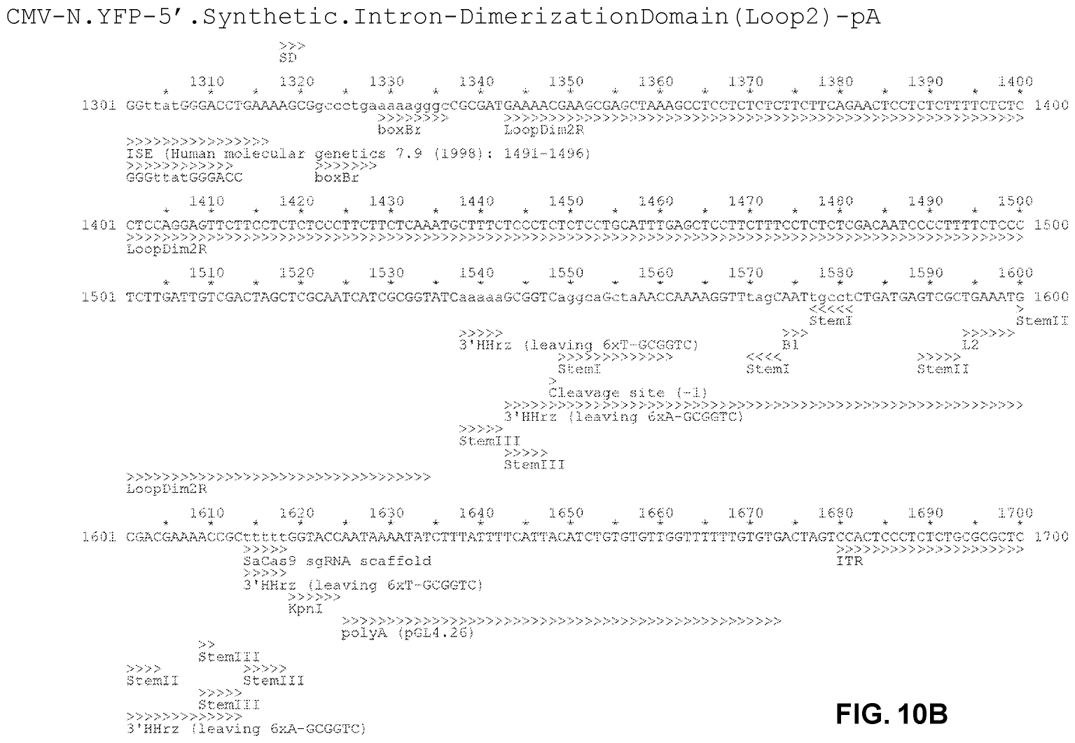

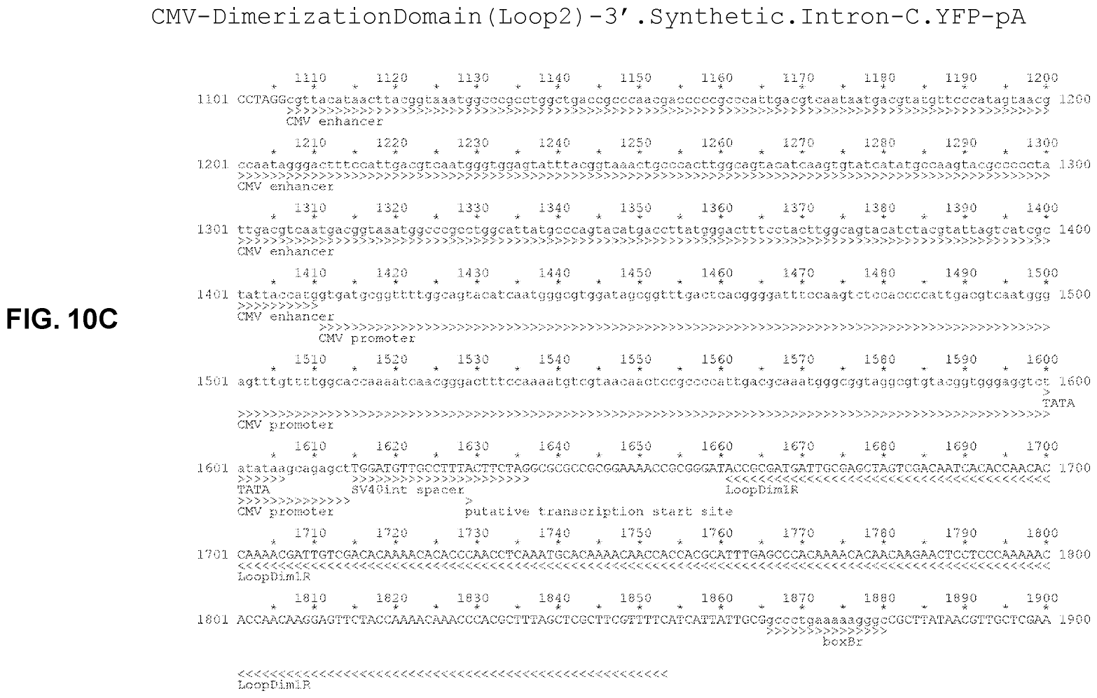

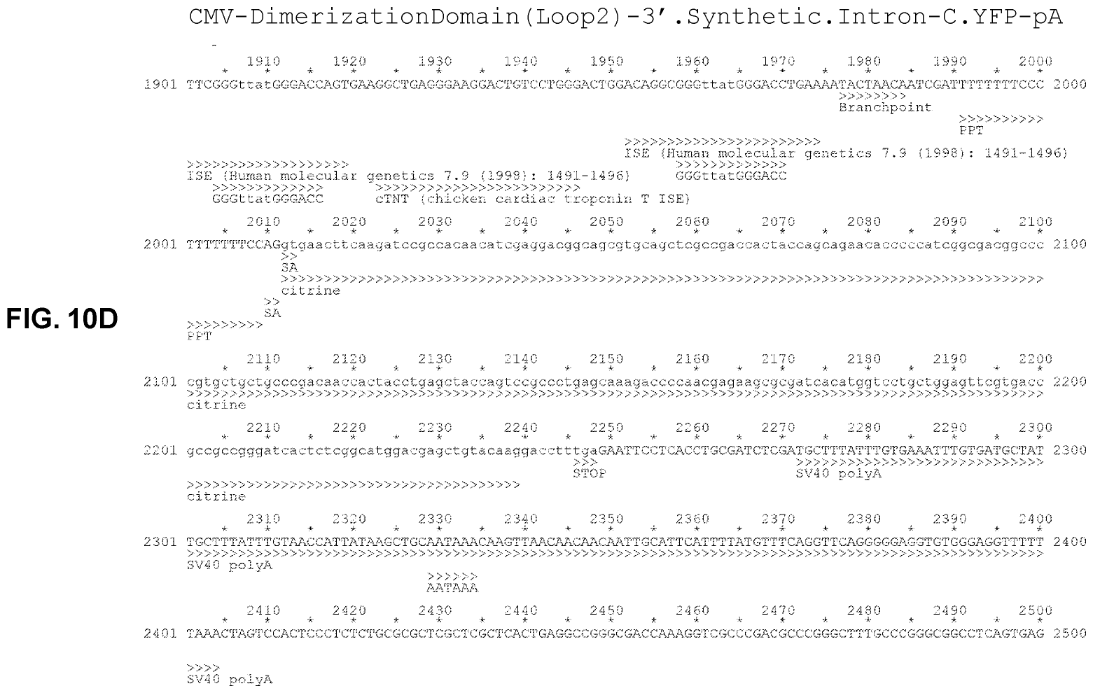

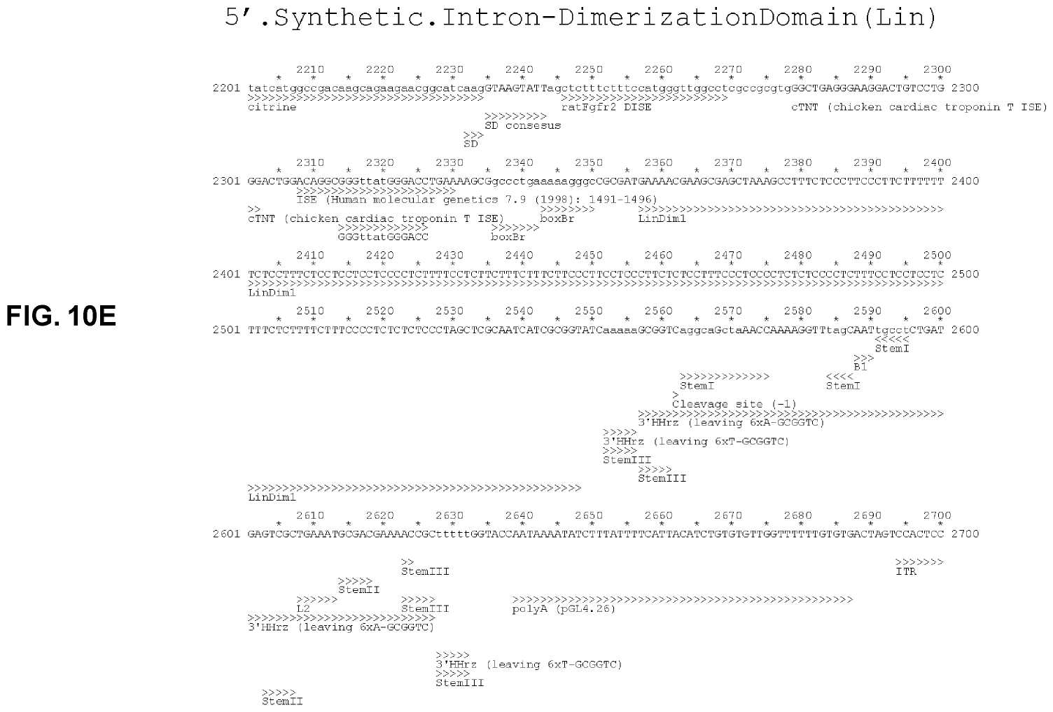

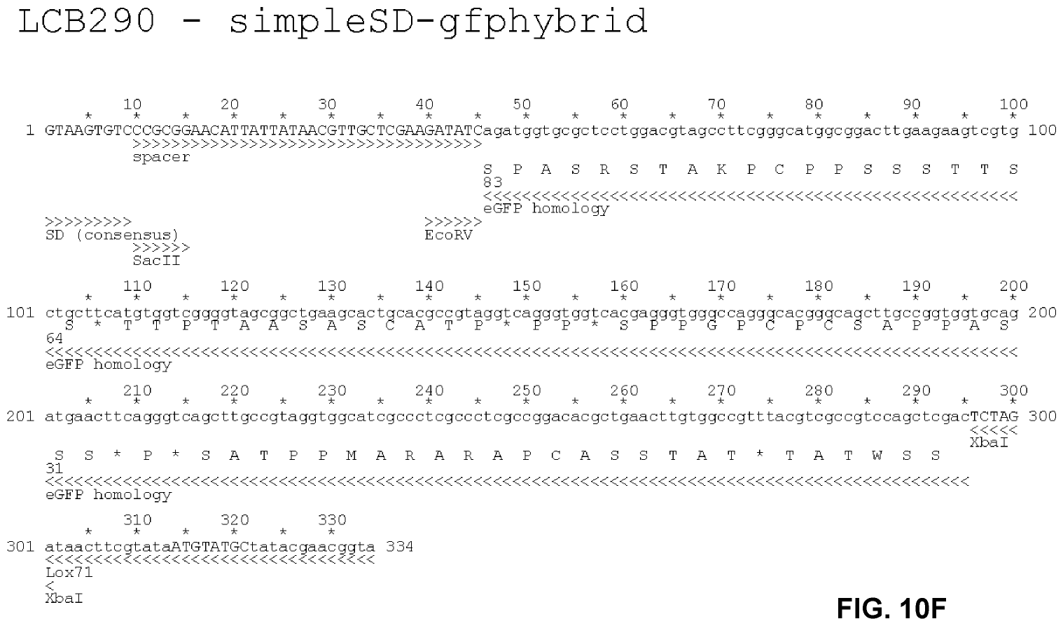

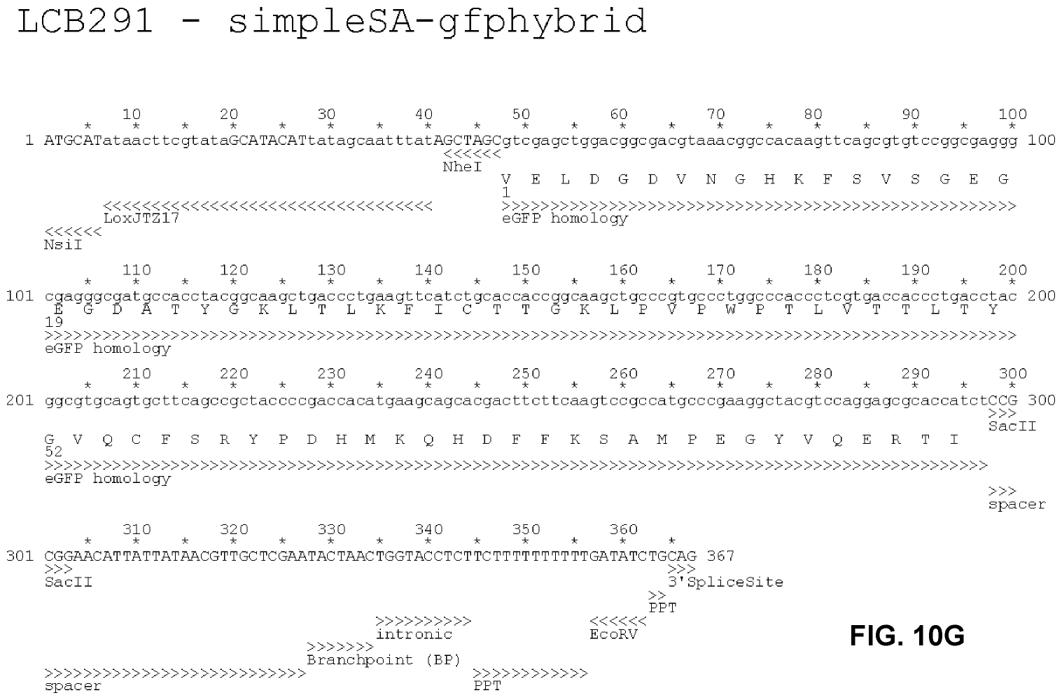

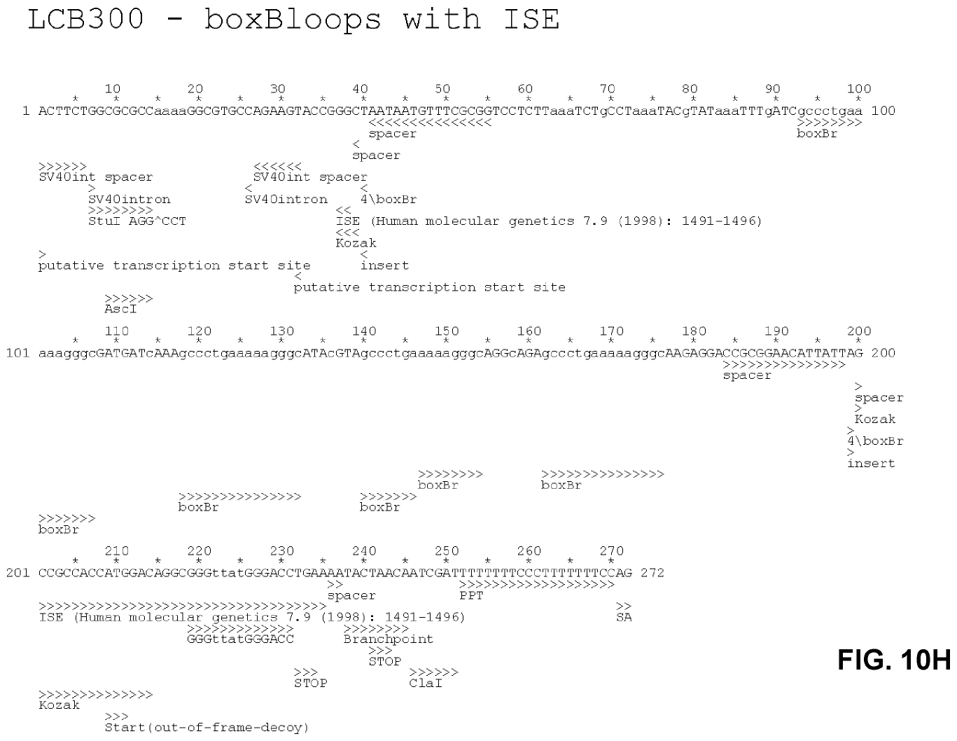

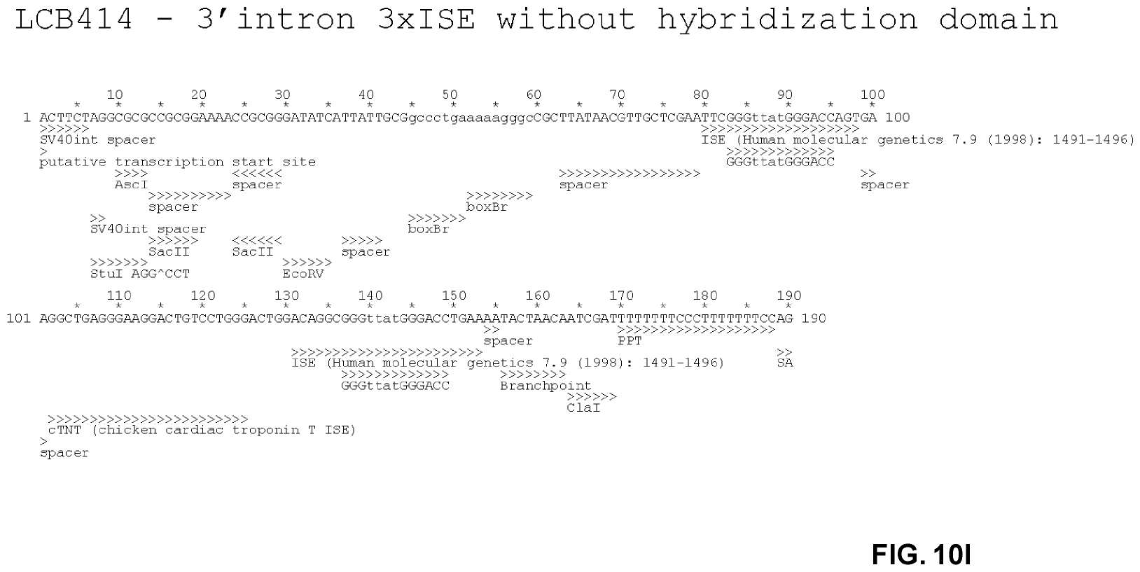

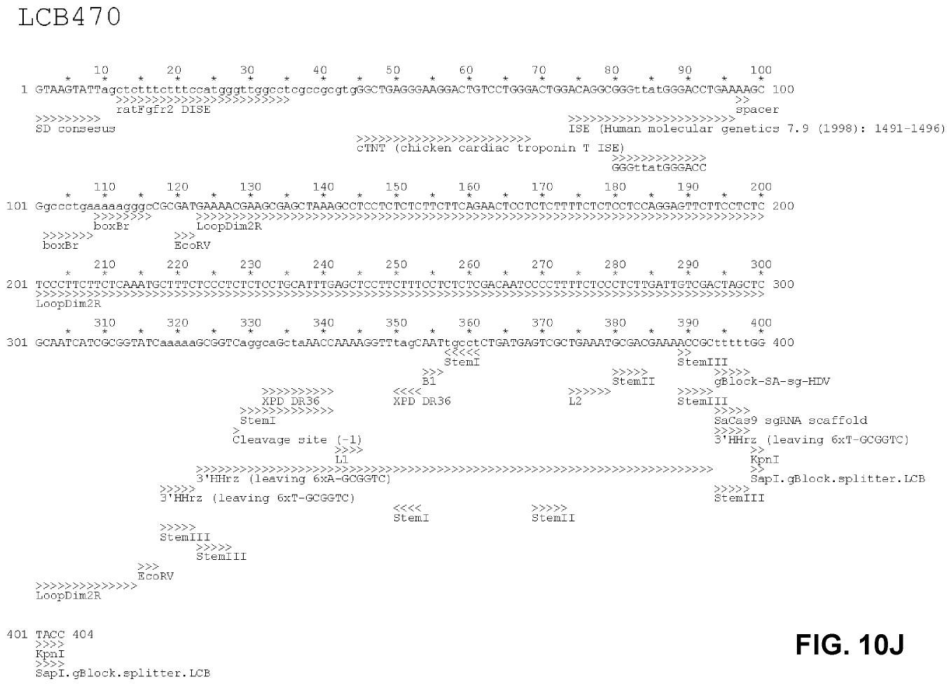

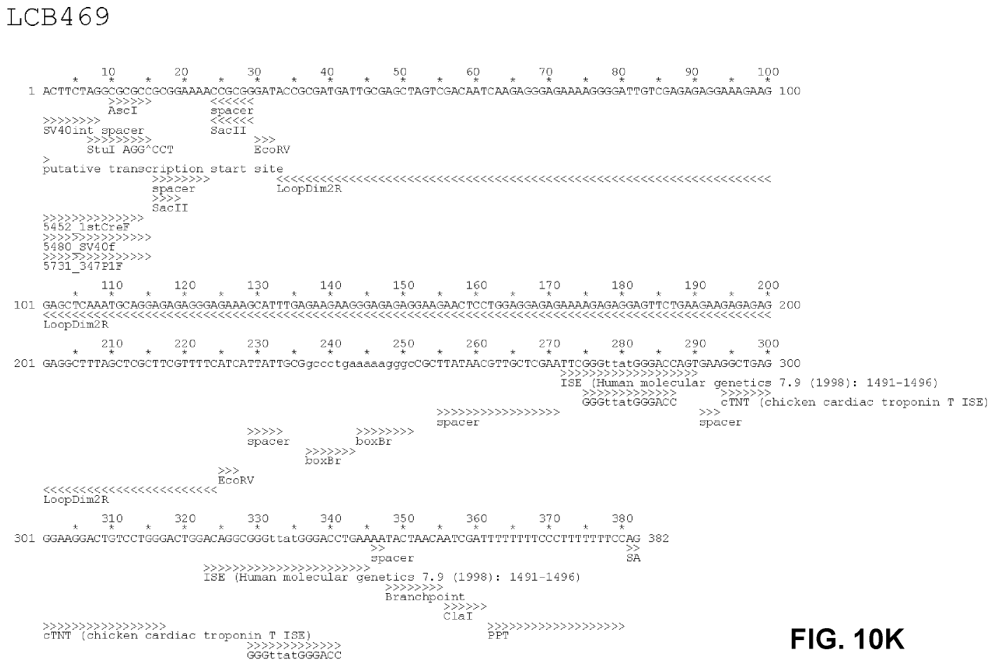

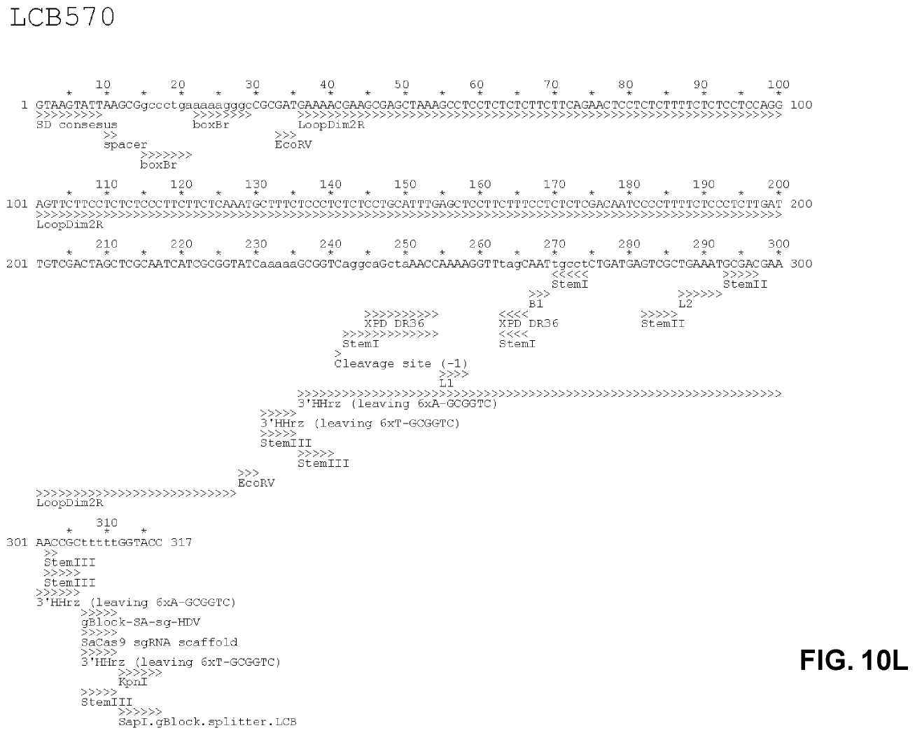

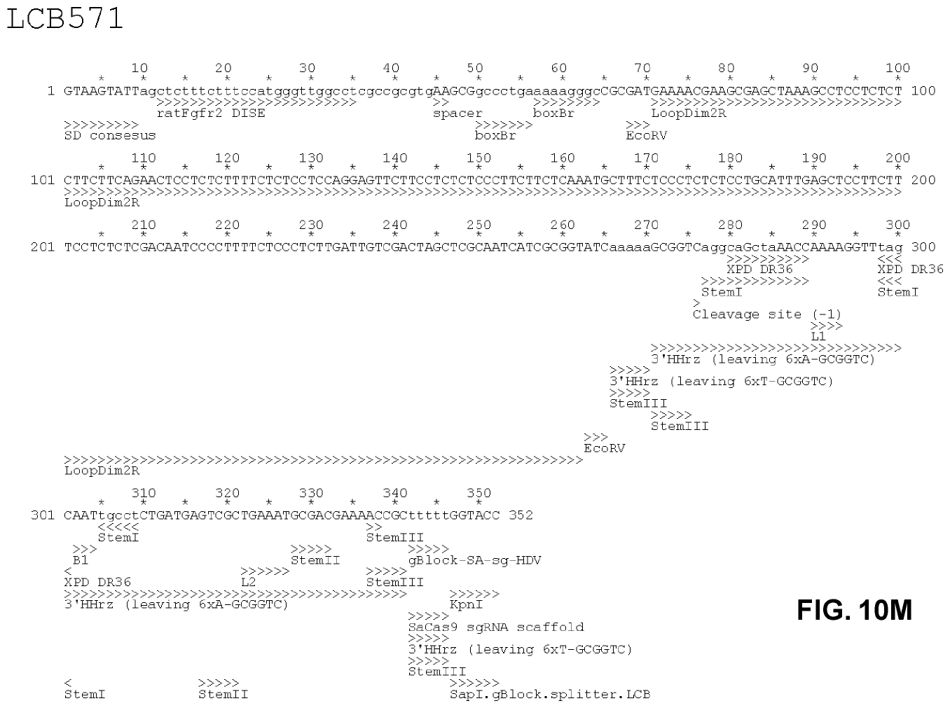

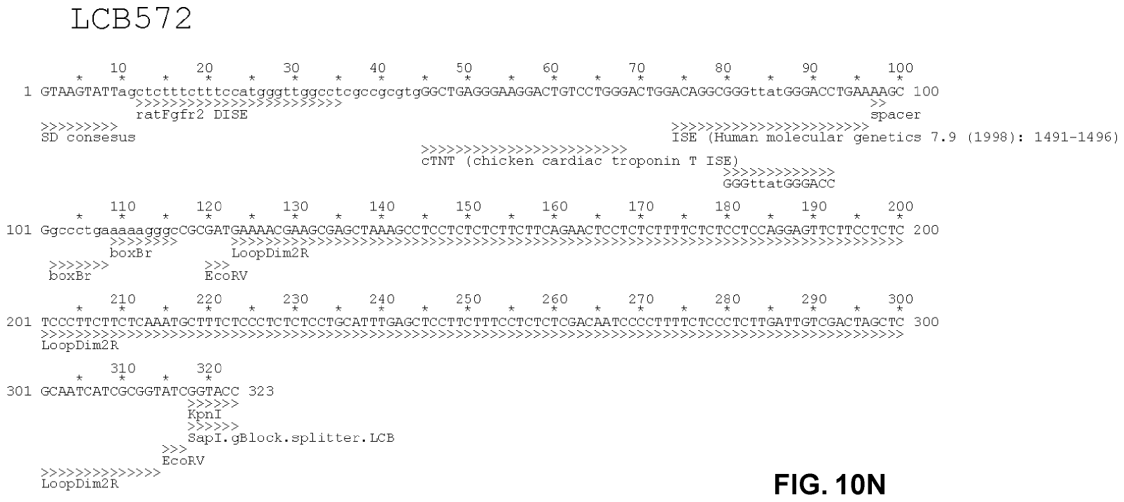

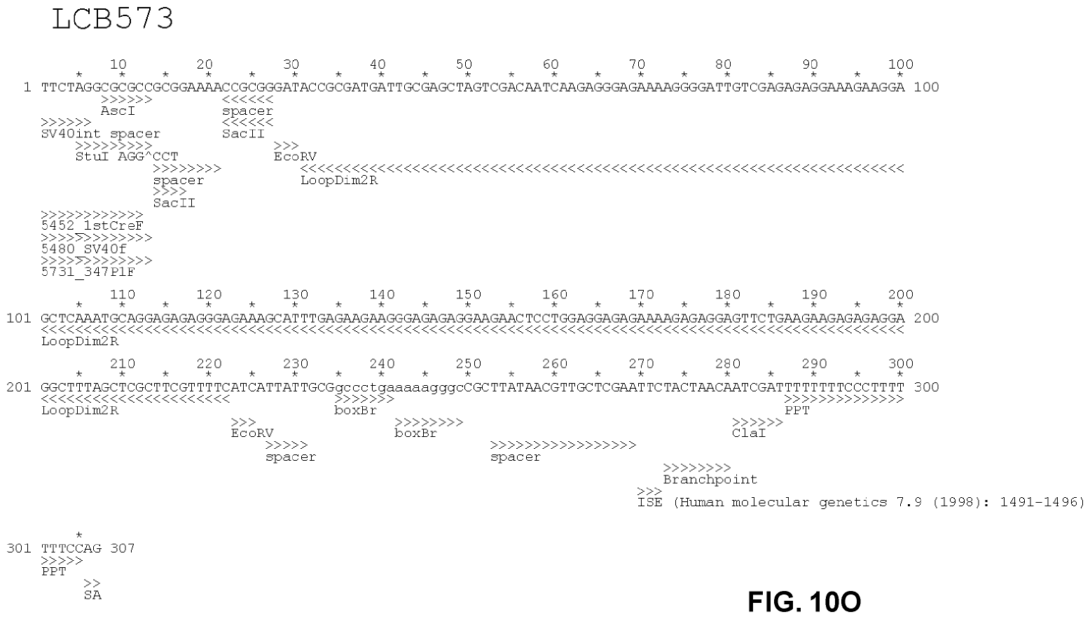

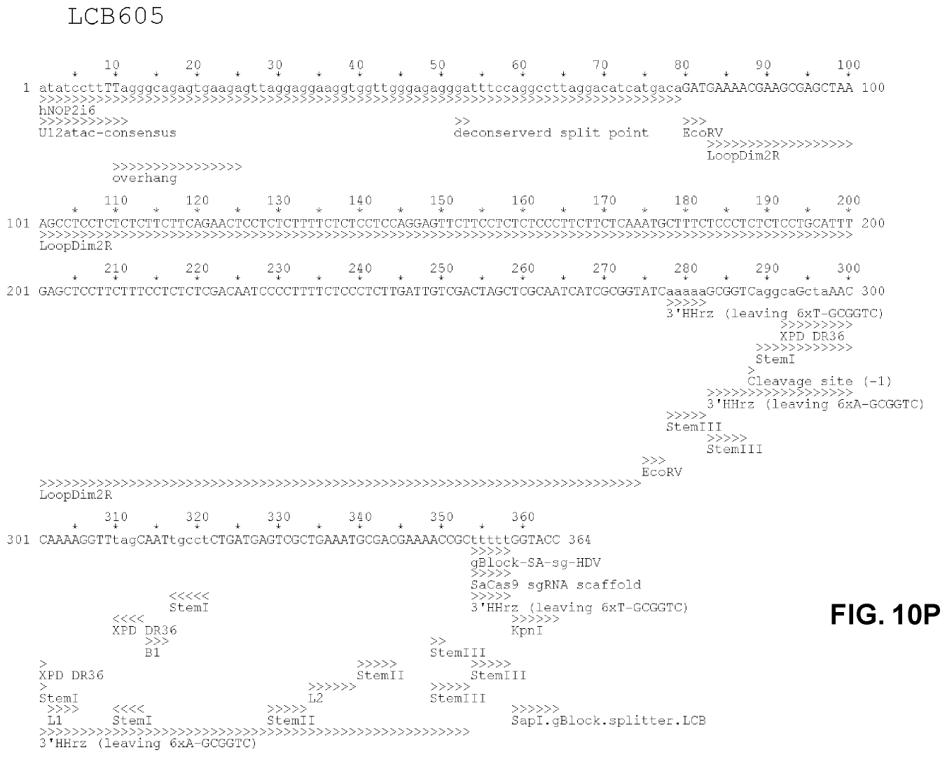

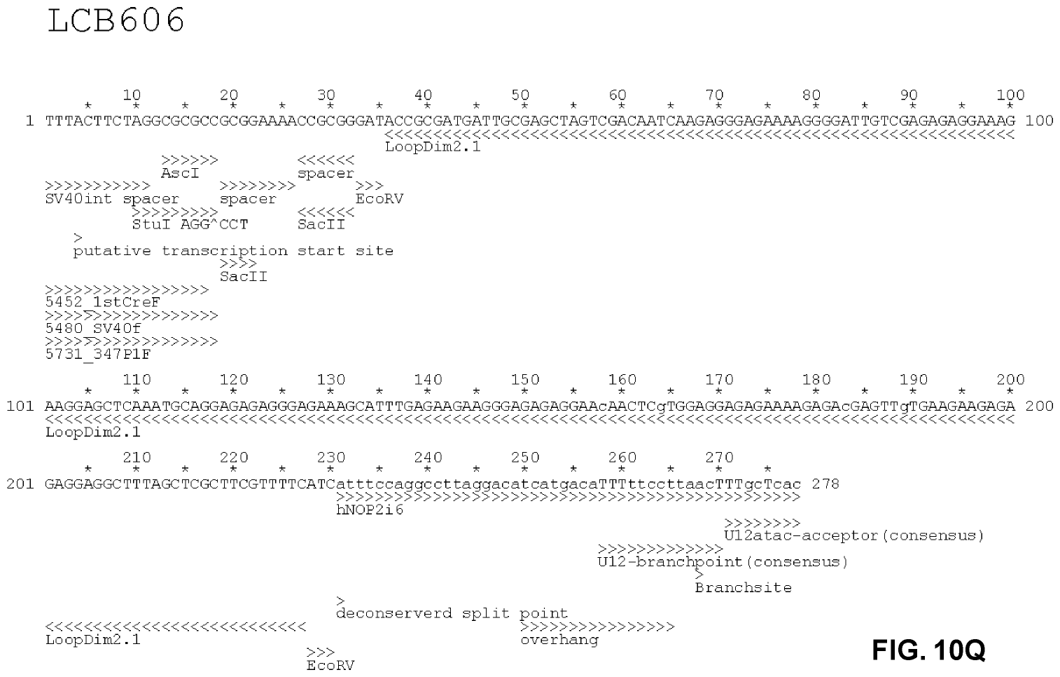

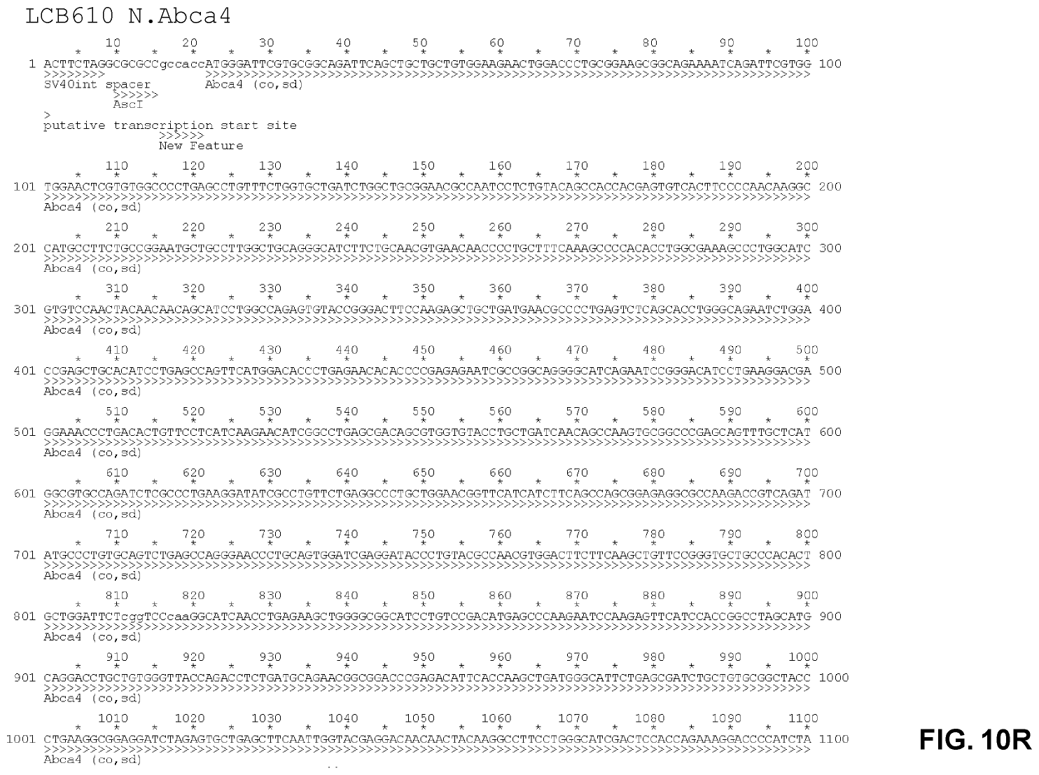

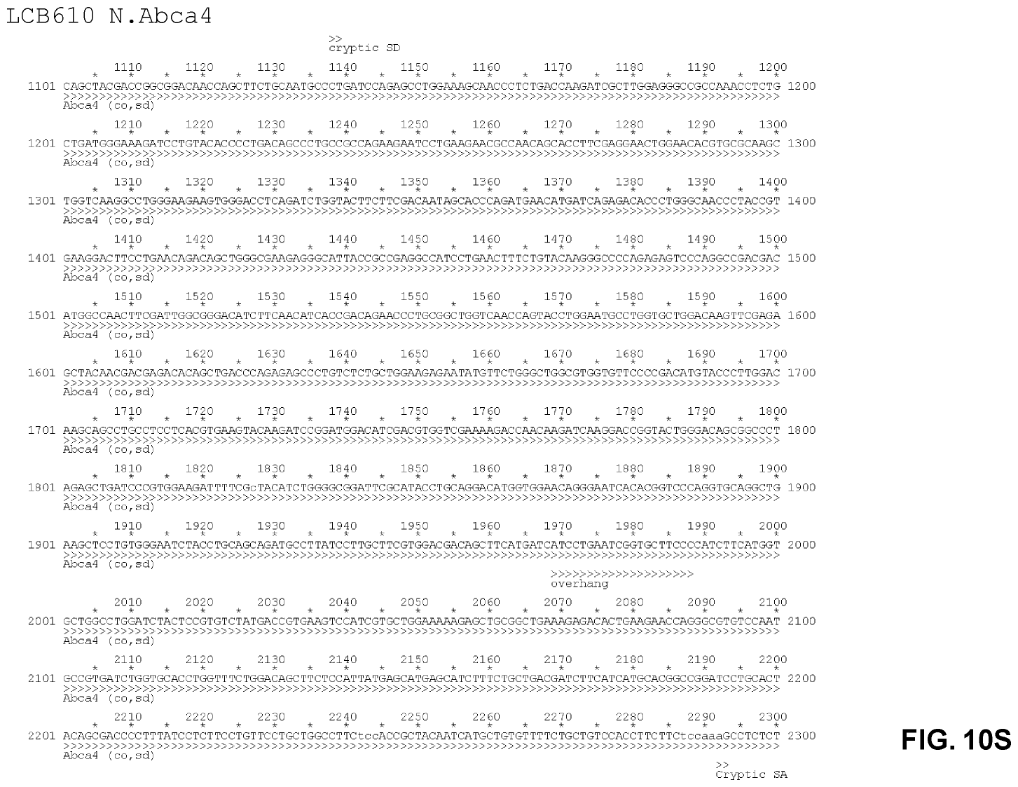

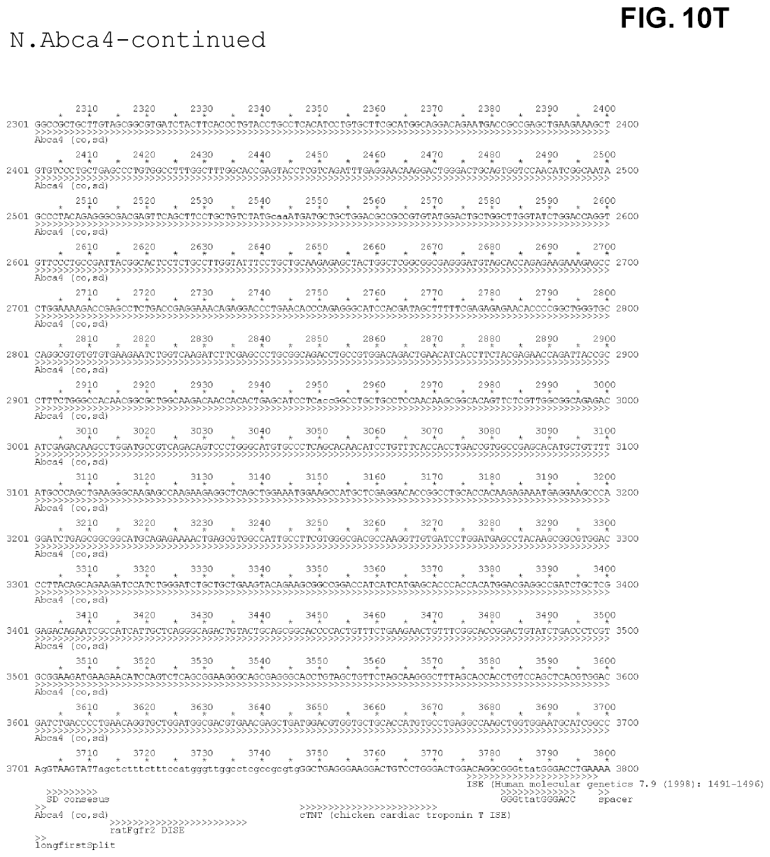

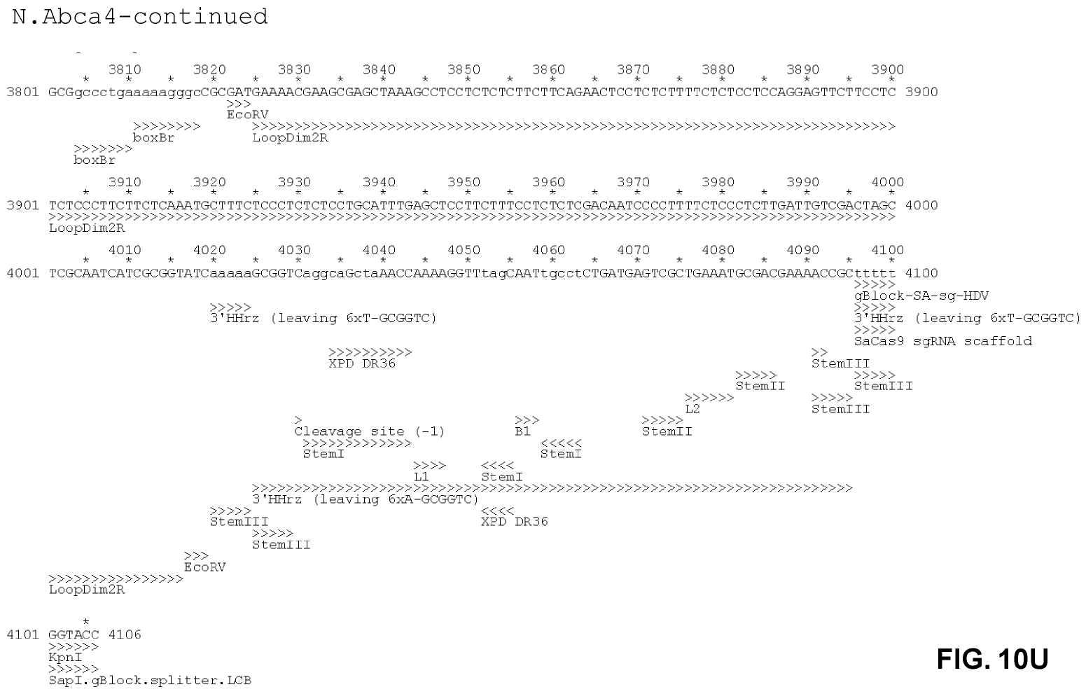

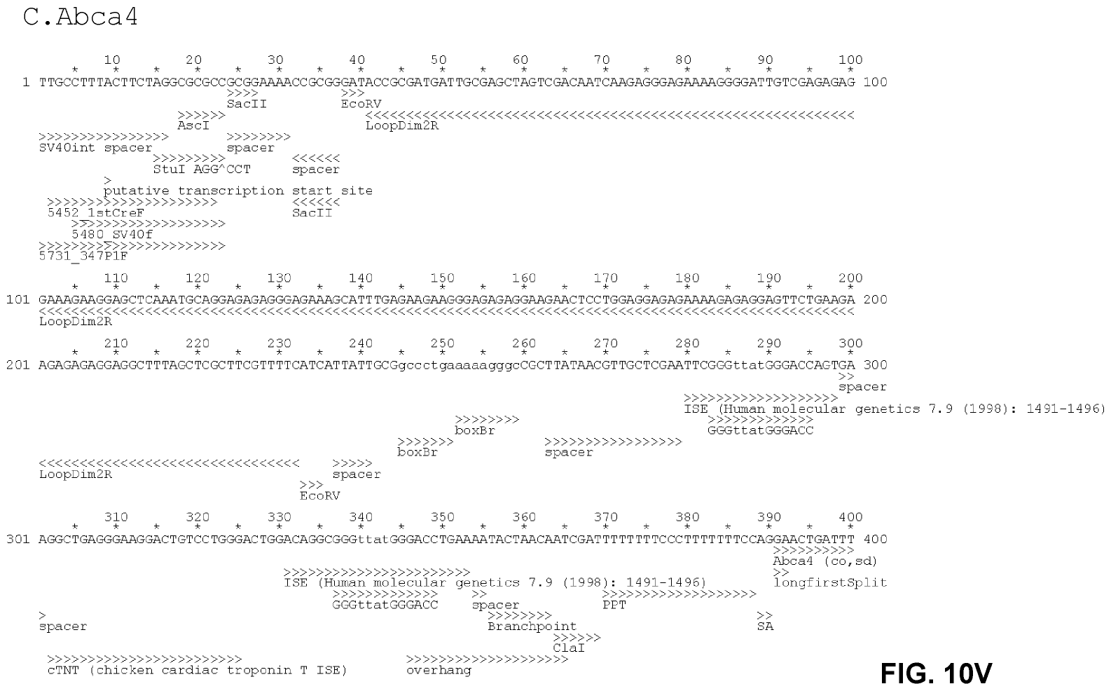

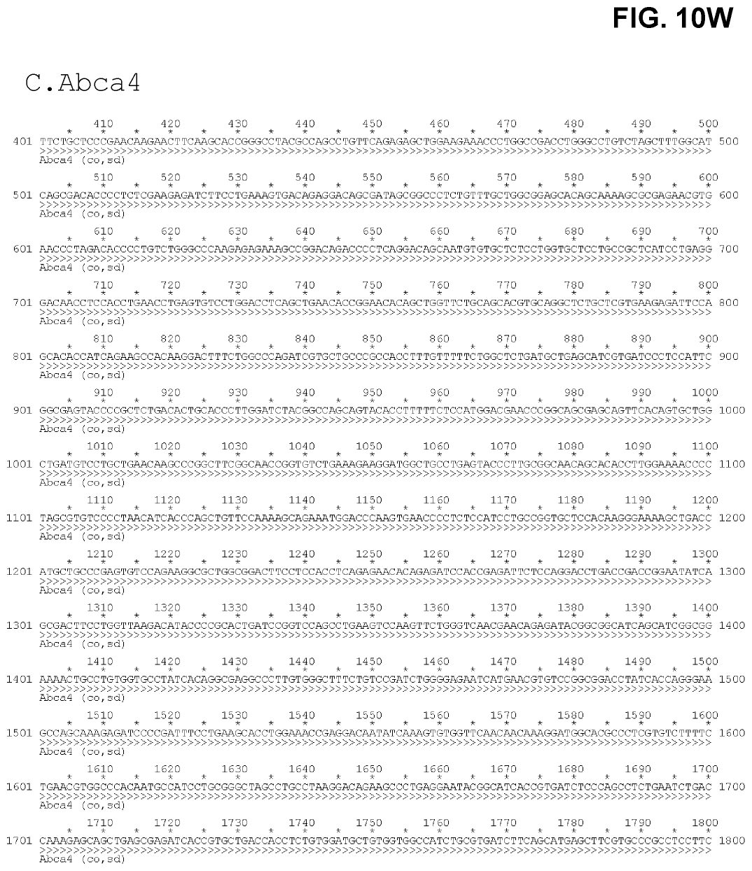

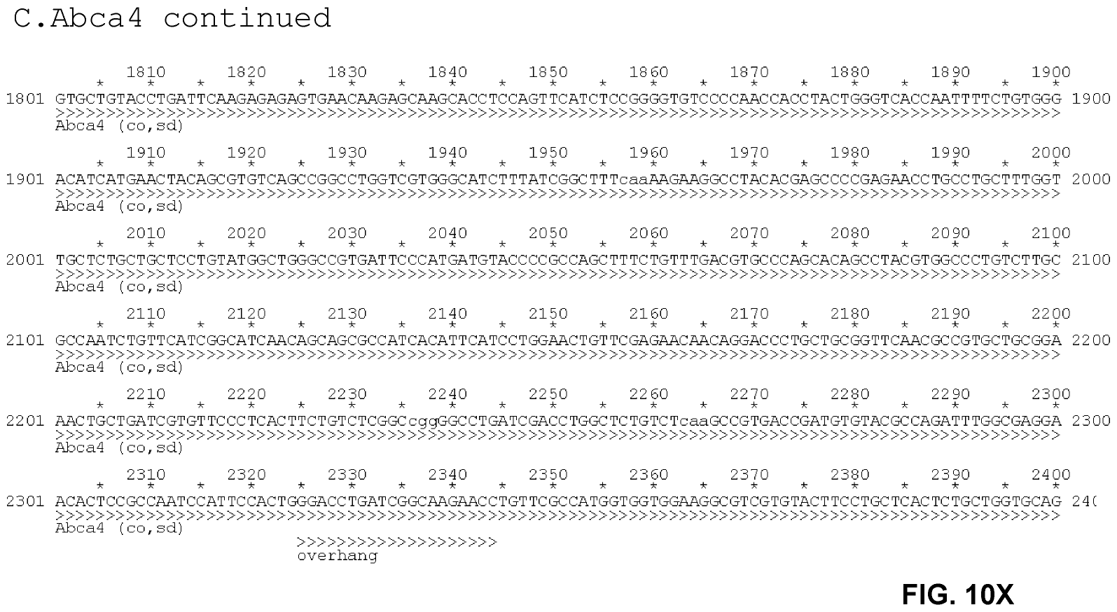

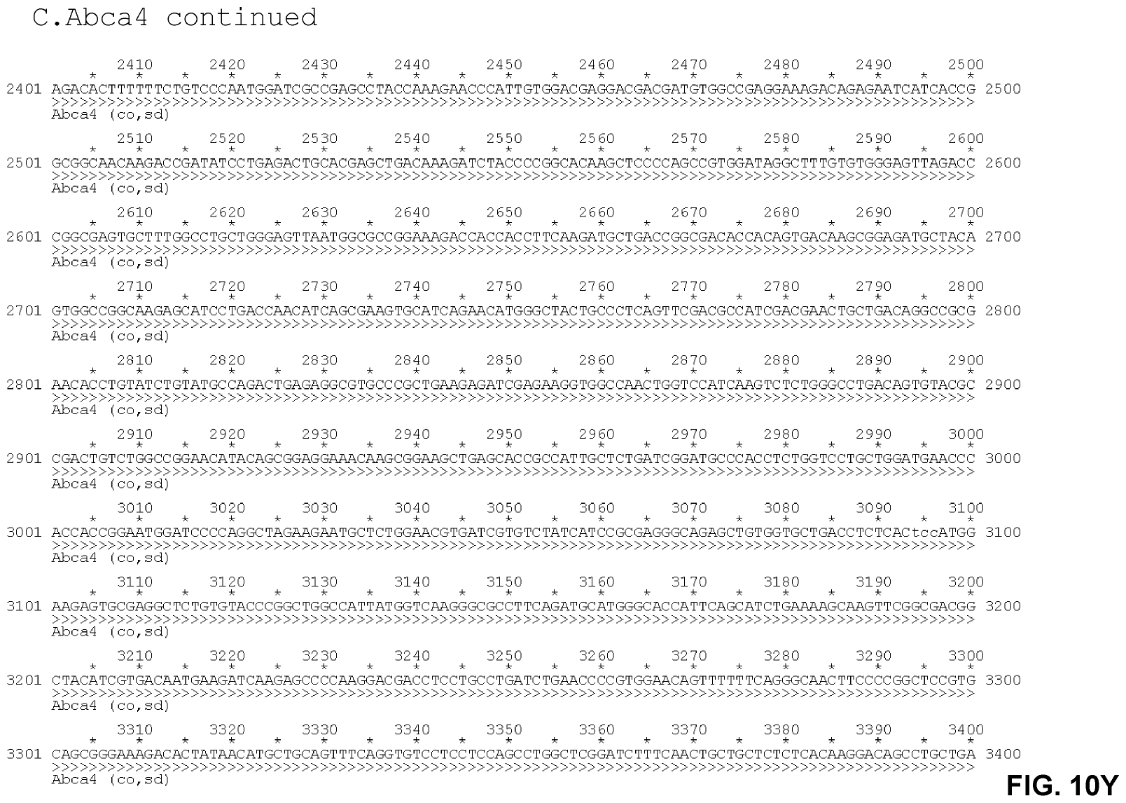

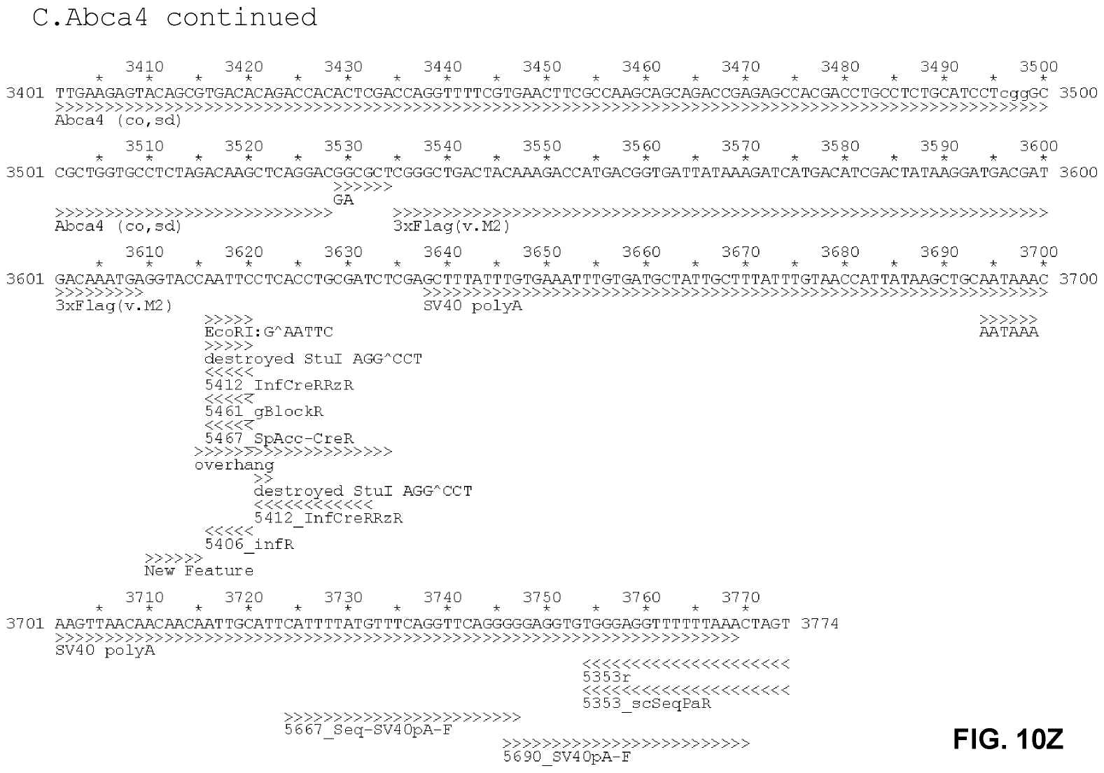

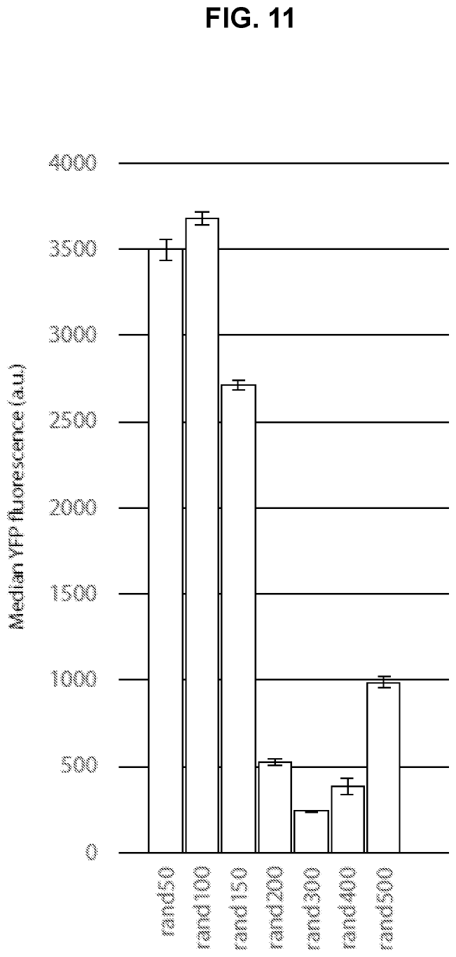

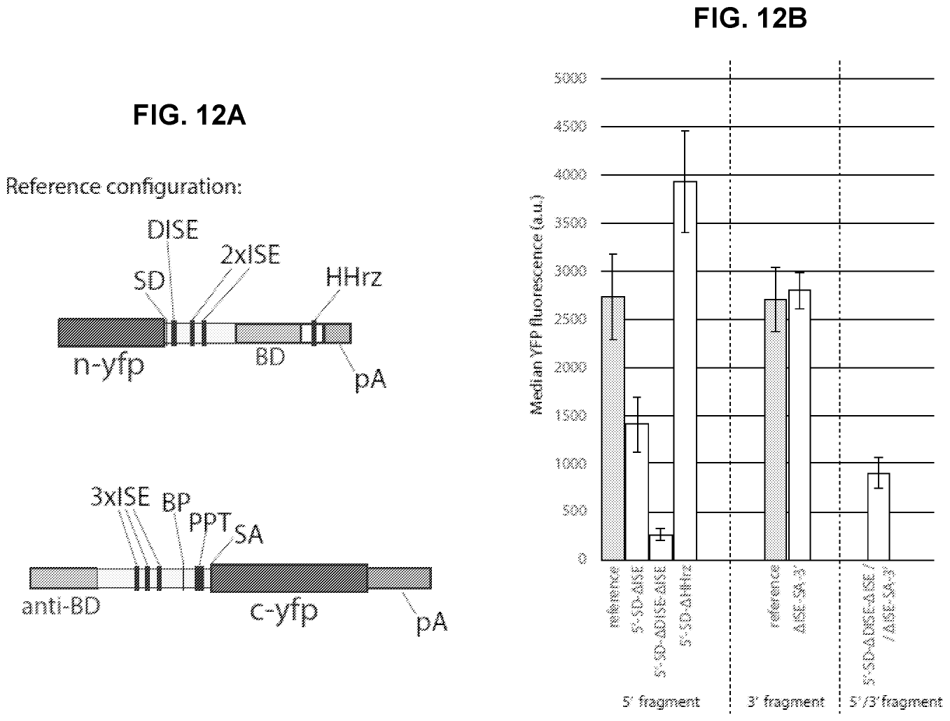

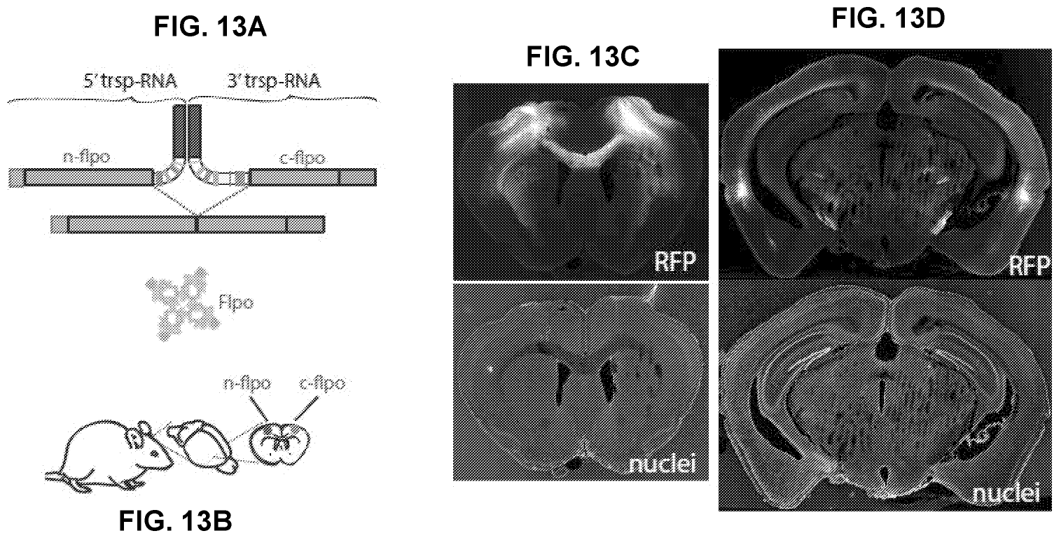

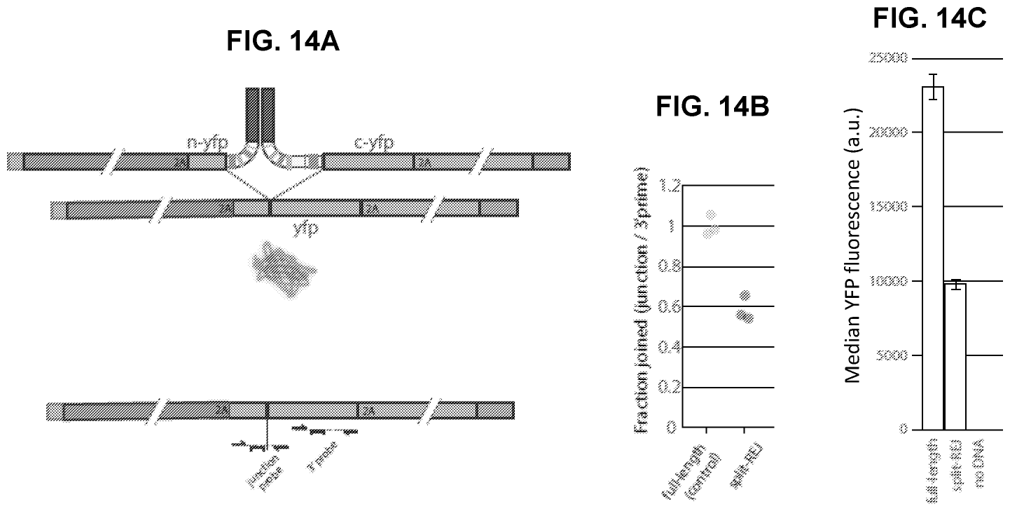

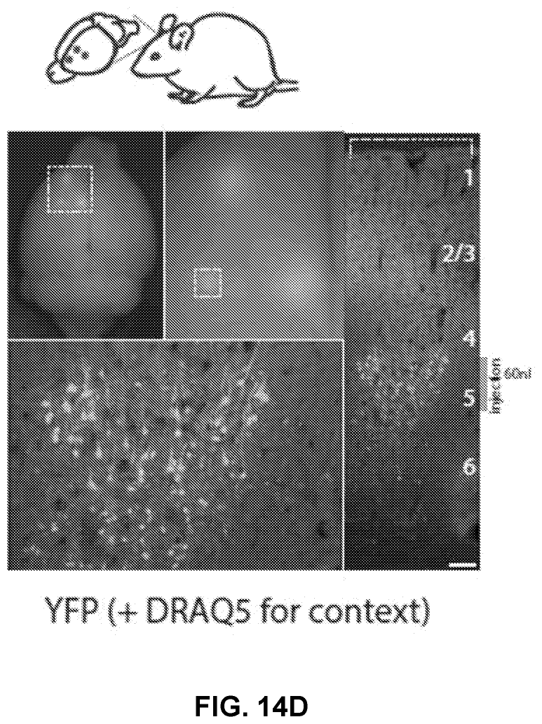

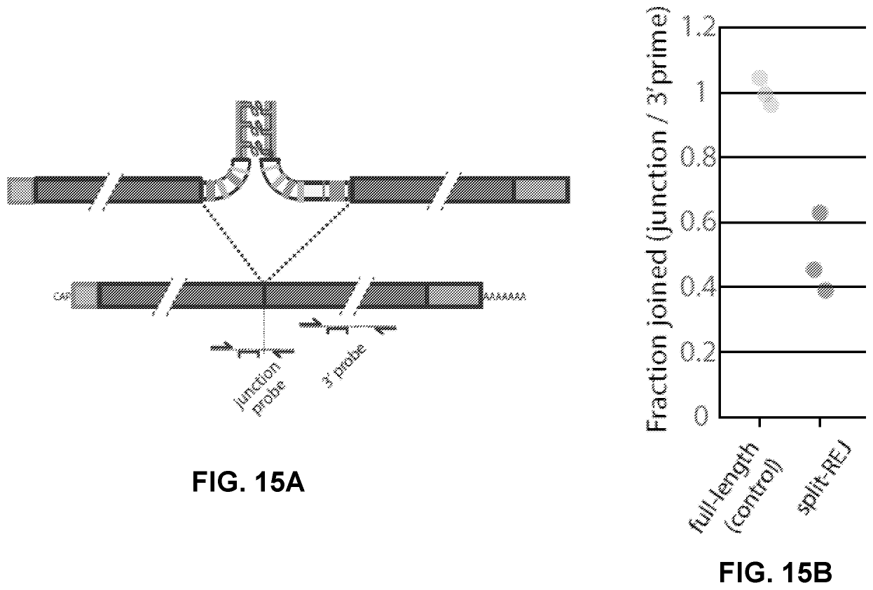

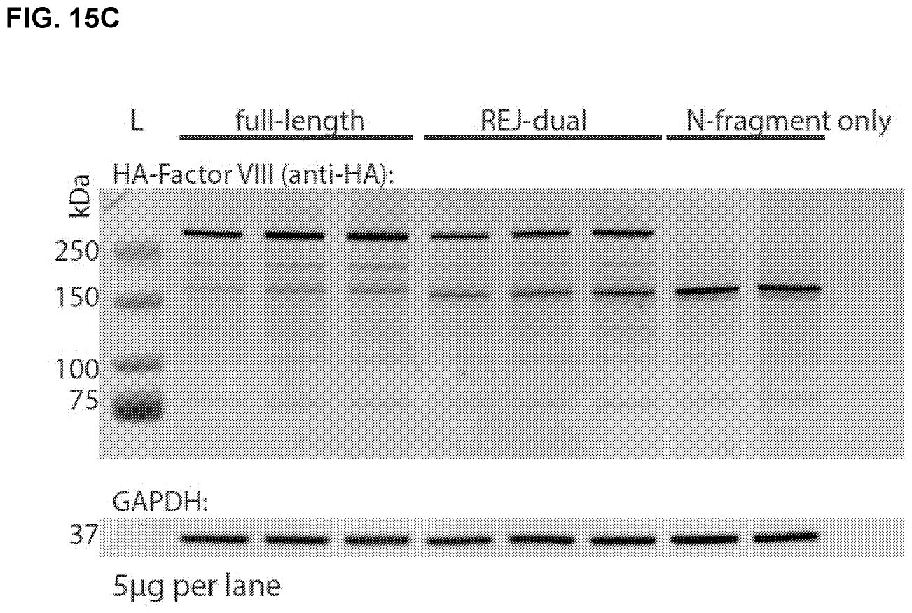

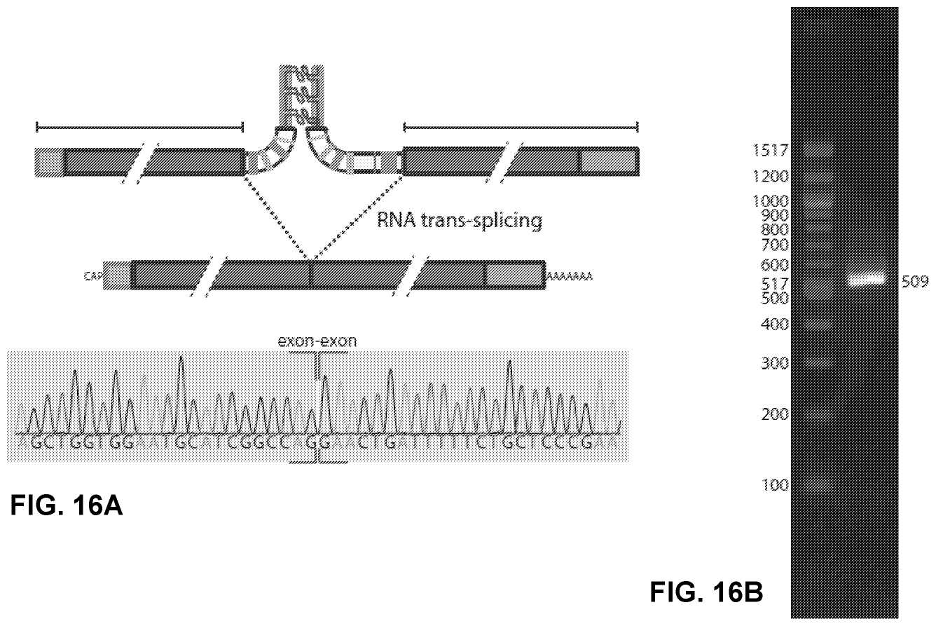

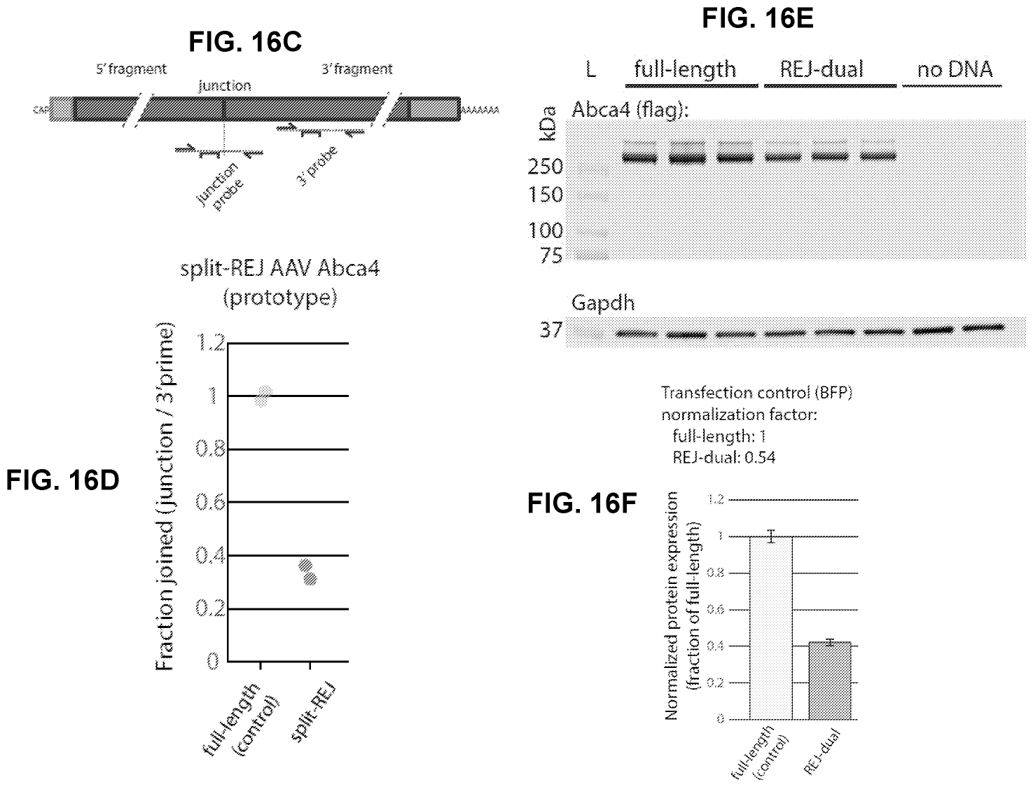

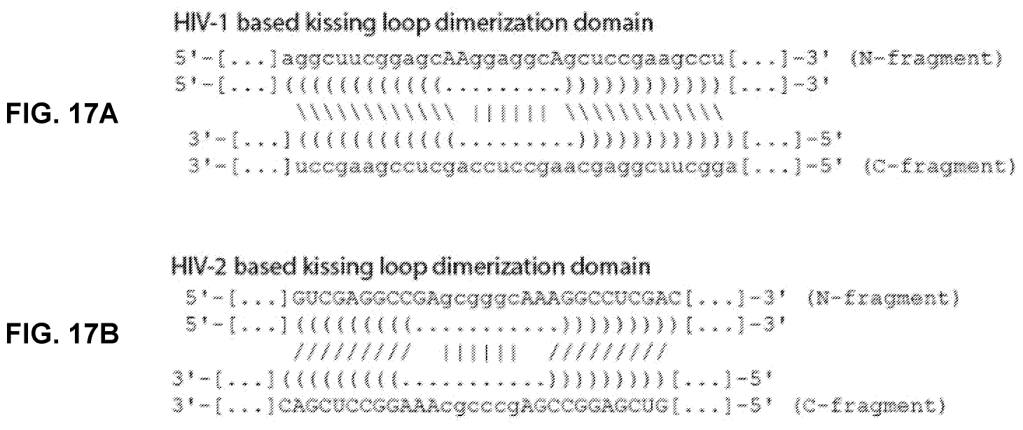

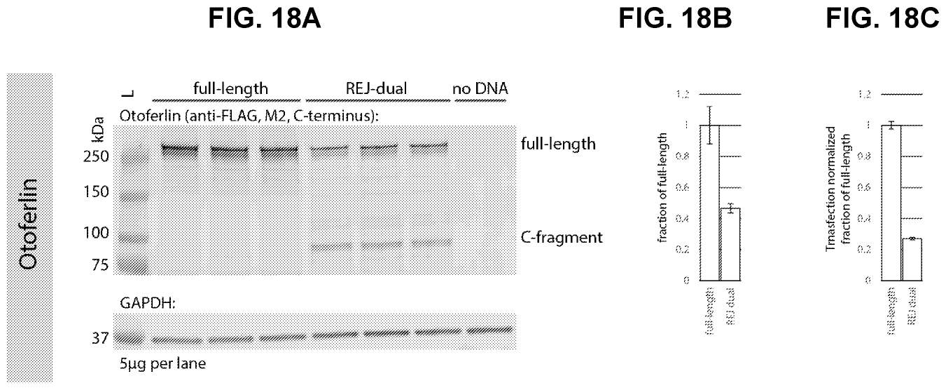

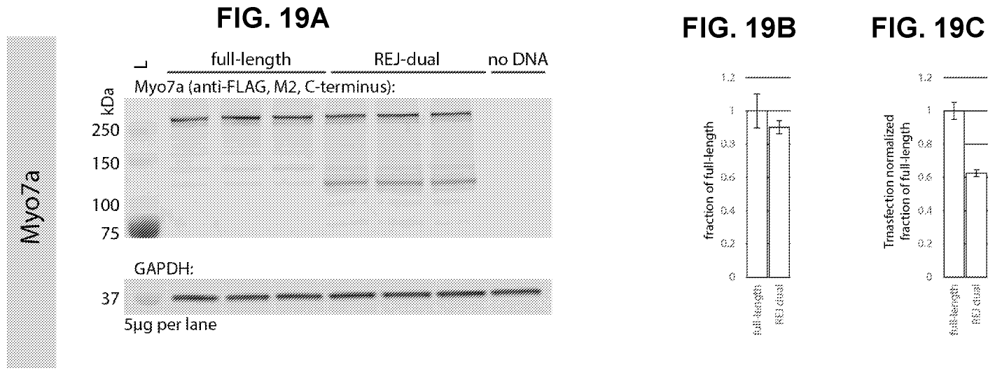

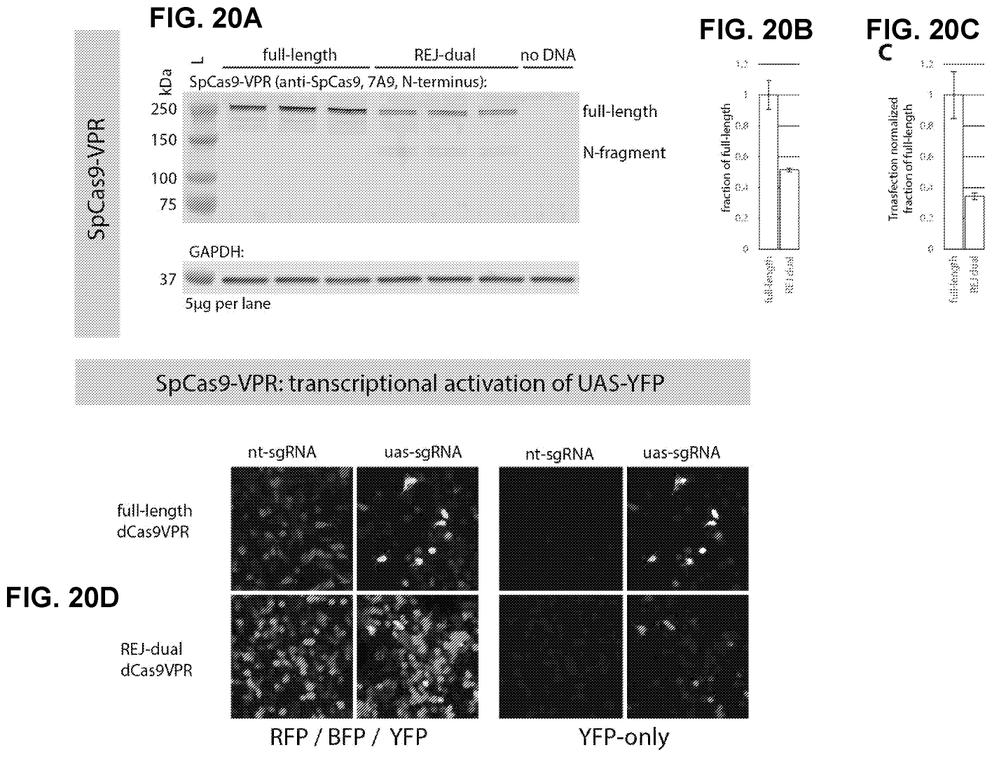

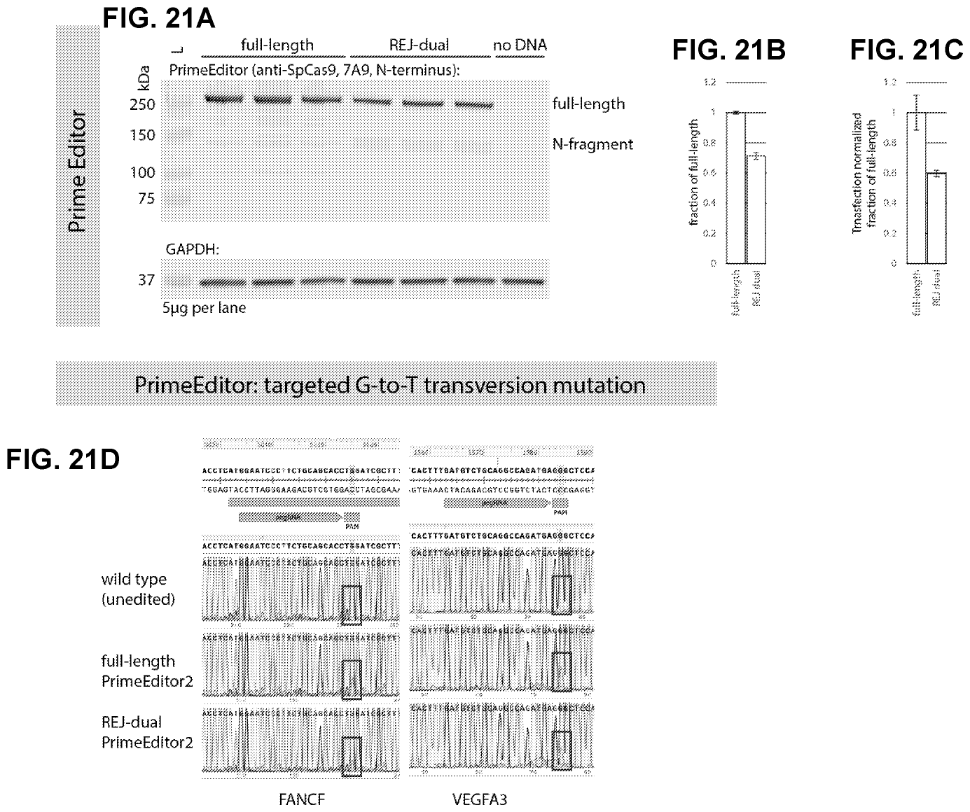

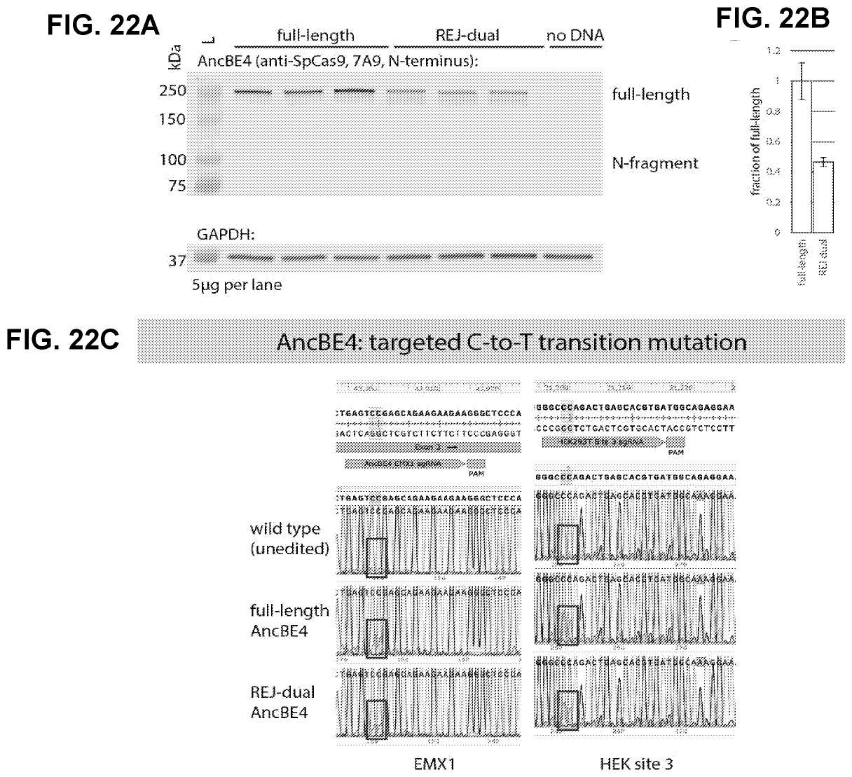

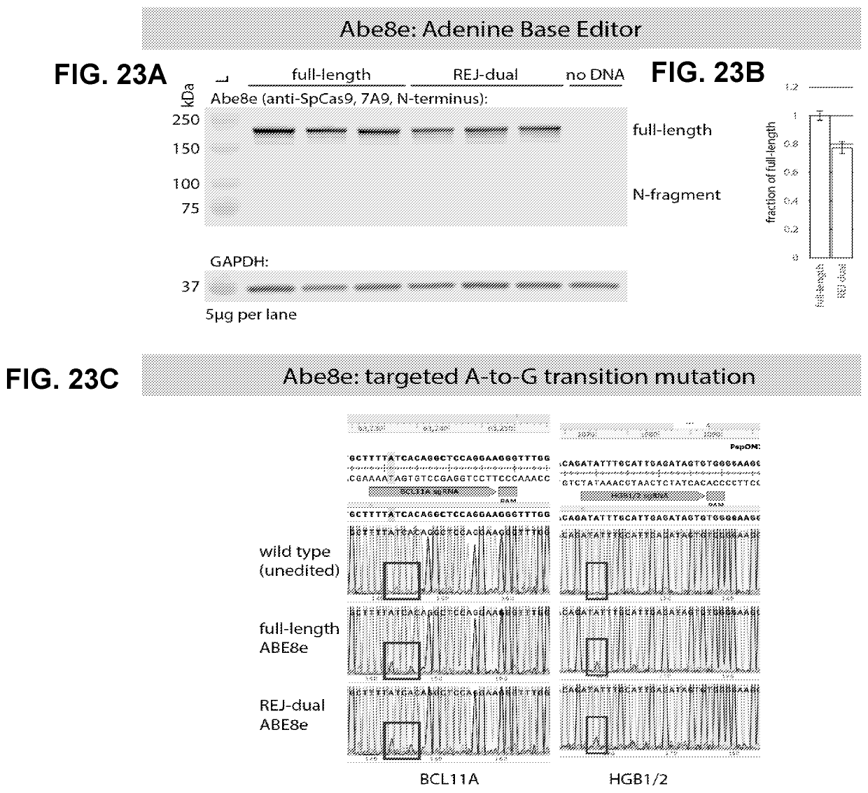

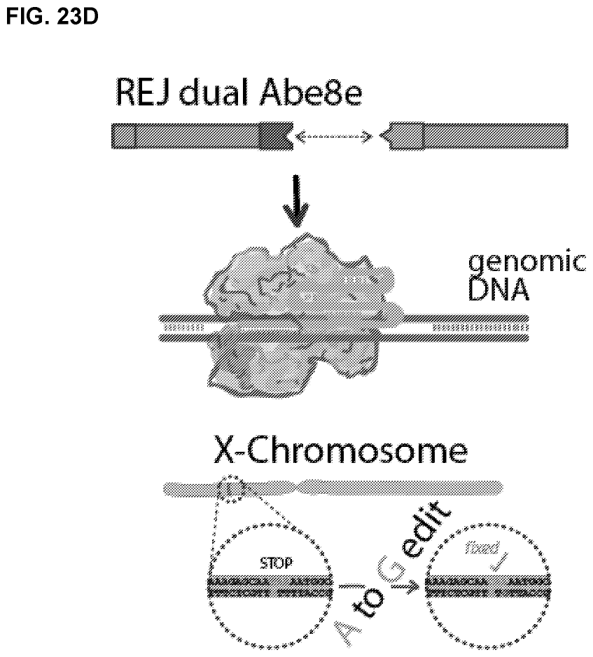

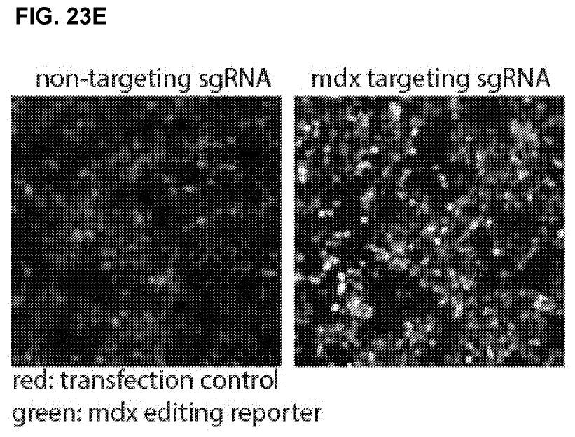

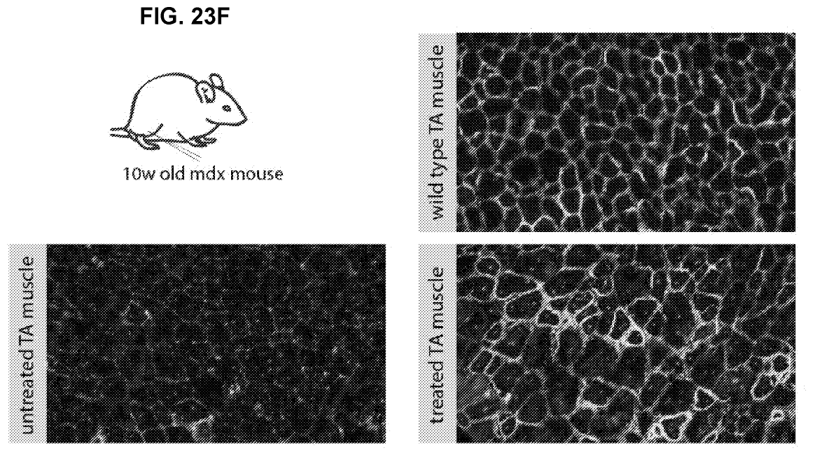

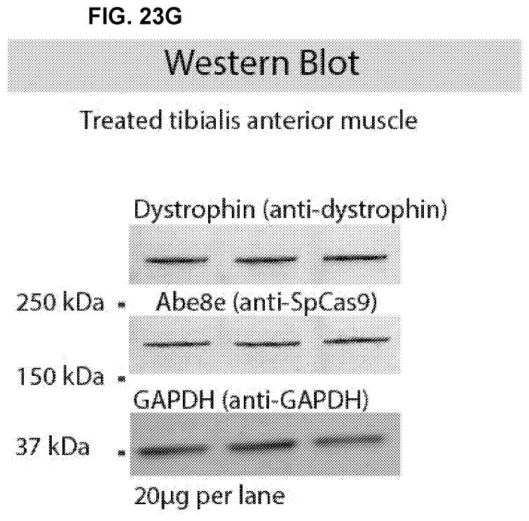

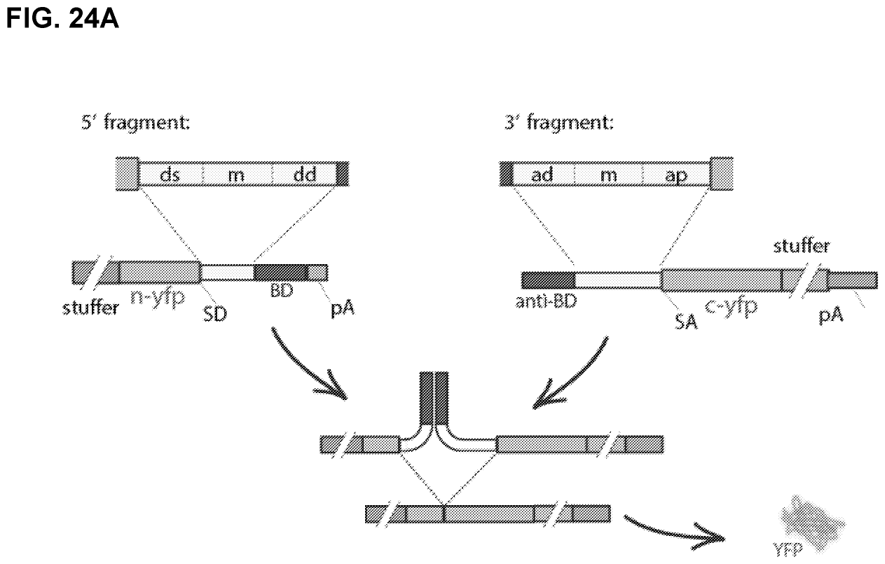

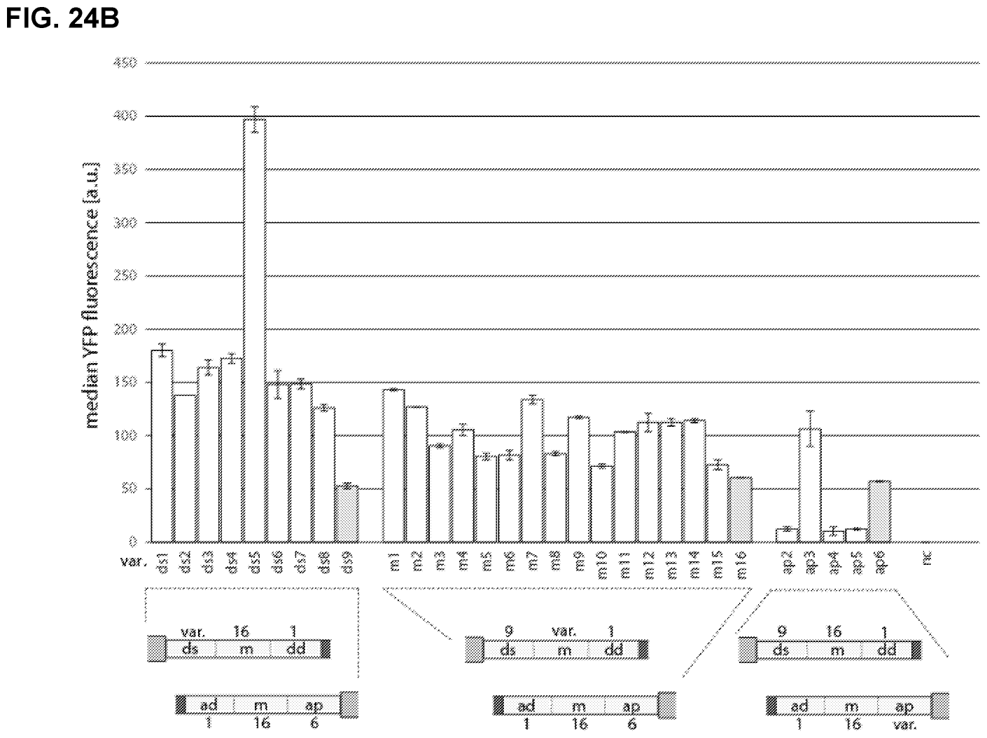

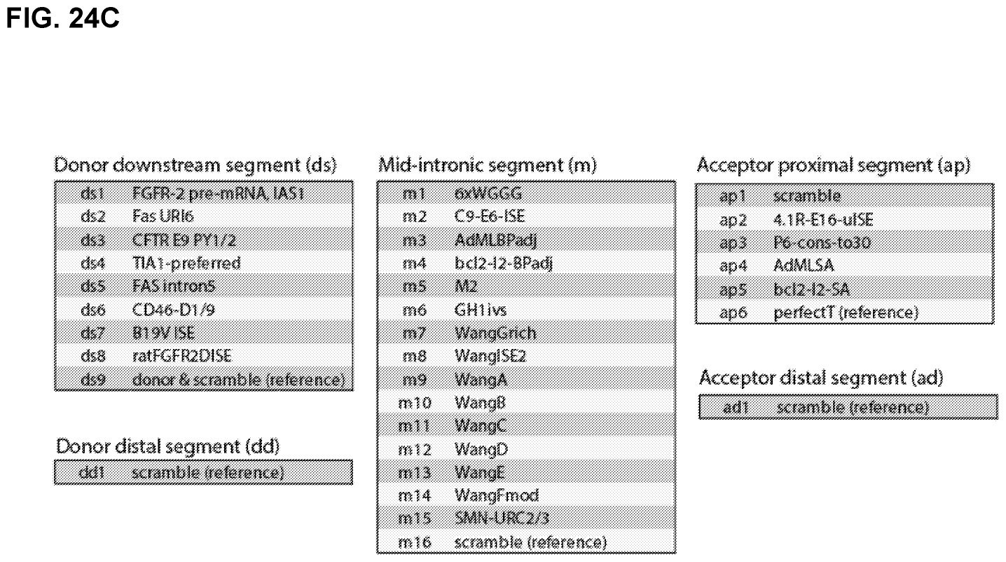

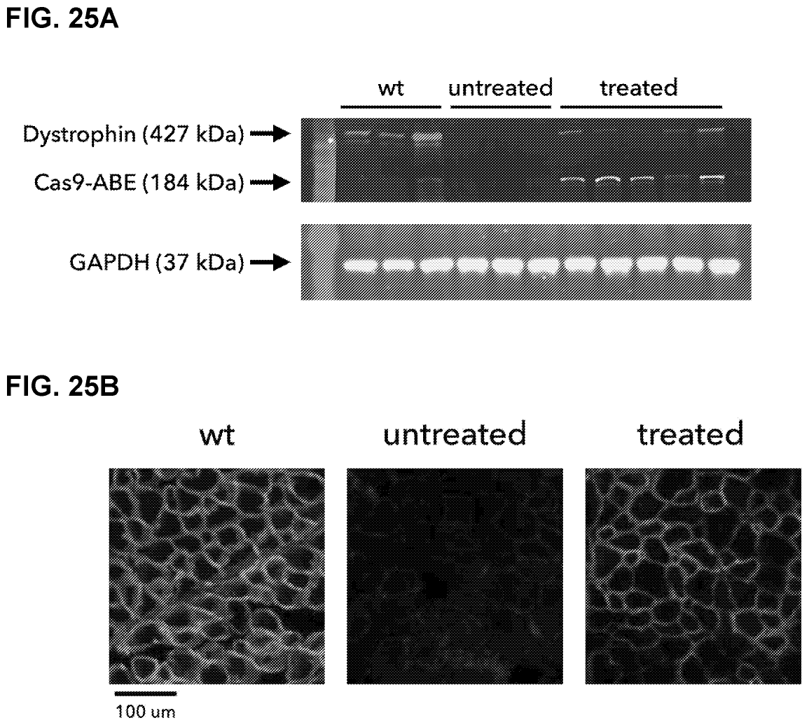

FIG. 6H is a schematic drawing providing an exemplary system or composition for the disclosed RNA recombination methods, using three DNA molecules 110, 200, 150, wherein the nucleic acid editing protein is divided into three portions (N-terminal, middle, C-terminal, 114, 216, 154, respectively) and each portion is encoded by a different nucleic acid molecule 110, 200, 150, respectively. In some examples, promoters 112, 202, 152 drive expression of each coding sequence 114, 216, 164. The system in this example includes one or more guide nucleic acid molecules (e.g., gRNA or gRNA coding sequence) 140, 141, 231, 232, 171, 172 which are specific for one or more target nucleic acid molecules. However, such guide nucleic acid molecules 140, 141, 231, 232, 171, 172 are optional, and in some examples instead of being provided as part of molecules 110, 200, 150, are provided separately, for example as part of a separate vector. This example shows optional guide nucleic acid molecules 140, 141, 231, 232, 171, 172 near the 5′ and 3′-ends of nucleic acid molecules 110, 200, 150, but the disclosure is not limited to such locations. If present, expression of one or more guide nucleic acid molecules 140, 141, 231, 232, 171, 172 can be driven by promoters 142, 143, 233, 234, 173, 174. The system can optionally include a parvovirus inverted terminal repeat (ITR) 176, 177, 235, 236, 178, 179 at each 5′ and 3′-end of molecules 110, 200, 150.

Although this figure shows an embodiment where molecules 110, 150, 200 of the system are DNA, following transcription, nucleic acid molecules 110, 150, 200 of the system are RNA, and thus lack the guide nucleic acid molecules 140, 141, 171, 172, 321, 232 promoters 112, 152, 202, 142, 143, 233, 234, 173, 174 and parvovirus ITR 176, 177, 235, 236, 178, 179. Drawing not to scale.

FIG. 7A is a schematic drawing providing an exemplary system for the disclosed RNA recombination methods, that like FIG. 6A uses two nucleic acid molecules 500, 600, but the dimerization domains are aptamers 512, 602, that recognize the same target molecule 700. In some examples, for example following transcription, the elements shown are RNA. Drawing not to scale.

FIG. 7B is a schematic drawing providing an exemplary system for the disclosed RNA recombination methods, that, related to FIG. 7A, uses dimerization domains that recognize the same target molecule. Here, the target recognized by the dimerization domain is a specific RNA molecule (instead of molecule 700 in FIG. 7A, e.g., protein or small molecule). Each domain recognizes a different portion of an mRNA molecule only expressed in target cells (i.e., cells where target protein expression is desired), such as a cancer-specific transcript. In some examples, for example following transcription, the elements shown are RNA. Drawing not to scale.

FIG. 7C is a schematic drawing providing an exemplary system for the disclosed RNA recombination methods, that like FIGS. 6A and 7A, uses two nucleic acid molecules 800, 900, and shows the dimerization domains 812, 902 hybridizing to an oligonucleotide 1000 that prevents the dimerization domains from interacting with one another, and therefore prevents or reduces recombination of the N-terminal coding sequence 802 and C-terminal coding sequence 914. In some examples, for example following transcription, the elements shown are RNA. Drawing not to scale.

FIG. 8 is a bar graph comparing reconstitution of YFP protein expression in the presence (w/) or absence (w/o) of a WPRE3 sequence in the 3′ untranslated region. N=3 replicates per sample are shown.

FIG. 9A is a schematic drawing providing an example for the use of dimerization domain (e.g., 122, 154 of FIG. 6A) that includes kissing loop interaction for high affinity dimerization. Using the teachings provided herein, one will appreciate that any of the disclosed coding portions (e.g., YFP) can be replaced with other target protein coding sequences. Drawing not to scale.

FIG. 9B depicts RFP, BFP, and YFP signal in HEK293T cells transfected with both halves of the split YFP. Equipped with either a linear dimerization domain adhering to the hypodiverse design principle or a structured dimerization domain designed for kissing loop-loop interactions. Strong yellow fluorescent signal indicates efficient reconstitution.

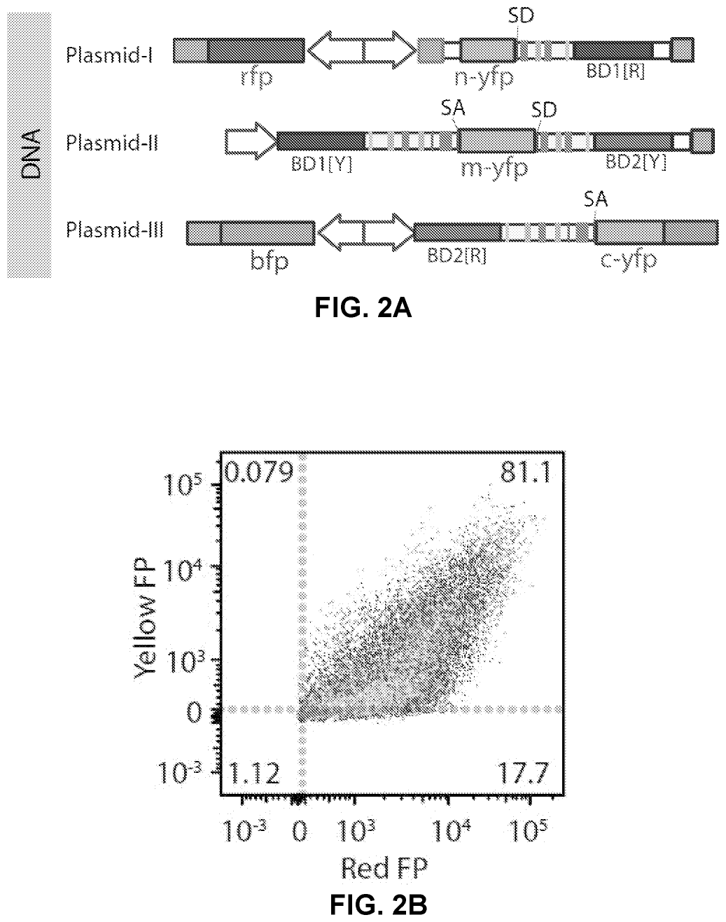

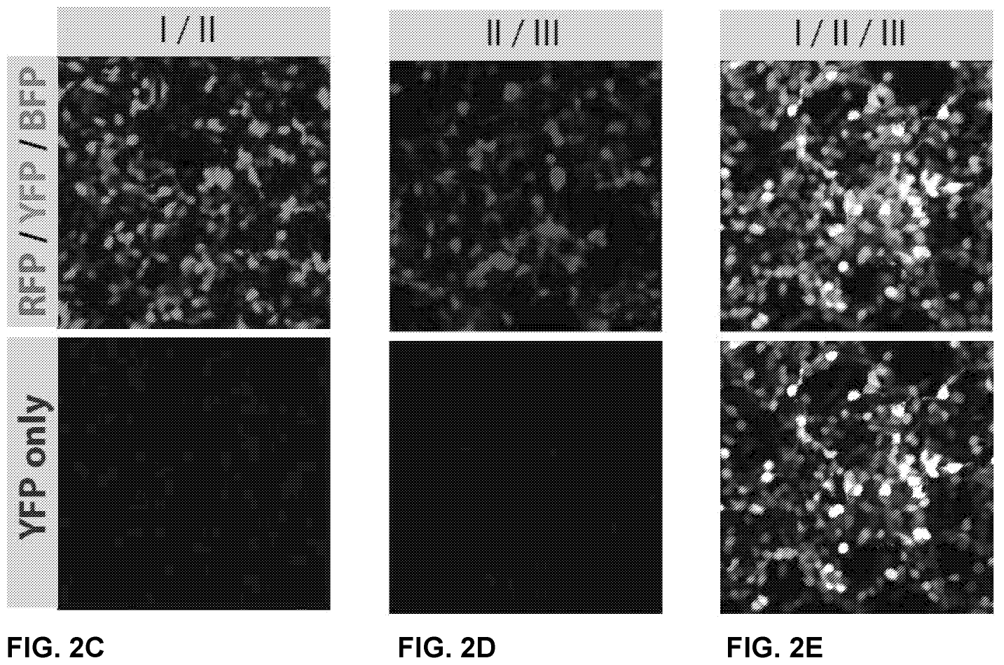

FIGS. 10A-10Z are exemplary synthetic nucleic acid molecules that can be used with the systems and methods. In some examples, a synthetic nucleic acid molecule as at least 80%, at least 85%, at least 90%, at least 95%, at least 98%, at last 99% or 100% sequence identity to the sequence of any one of SEQ ID NOS: 1 (FIGS. 10A-10B), 2 (FIGS. 10C-10E), 7 (FIG. 10E), 8 (FIG. 10F), 9 (FIG. 10G), 10 (FIG. 10H), 11 (FIG. 10I), 12 (FIG. 10J), 13 (FIG. 10K), 14 (FIG. 10L), 15 (FIG. 10M), 16 (FIG. 10N), 17 (FIG. 10O), 18 (FIG. 10P), 19 (FIG. 10Q), 20 (FIGS. 10R-10U), and 21 (FIGS. 10V-10Z), but with a different target protein coding sequence. Thus an intronic region used with any of the systems or methods provided herein can have at least 80%, at least 85%, at least 90%, at least 95%, at least 98%, at last 99% or 100% sequence identity to any intronic sequence of SEQ ID NOS: 1, 2, 3, 4, 7, 8, 9, 10, 11, 12, 13, 14, 15, 16, 17, 18, 19, 20, or 21. For example, FIGS. 10A-D show exemplary (A,B) first (SEQ ID NO: 1) and (C,D) second (SEQ ID NO: 2) synthetic molecules that can be used to express full-length YFP, while SEQ ID NO: 3 and 4 provide the corresponding synthetic intron portion without the YFP coding portion. In some examples, a synthetic intron sequence has at least 80%, at least 85%, at least 90%, at least 95%, at least 98%, at last 99% or 100% sequence identity to SEQ ID NO: 3 or 4. Thus, the coding sequence portion of any synthetic molecule provided herein (e.g., nt 544 to 1032 of SEQ ID NO: 1 and nt 905 to 1141 of SEQ ID NO: 2), can be replaced with another coding sequence portion.

FIG. 11 is a bar graph showing the reconstitution efficiency of different length random complimentary base-pairing binding domains (50 bp, 100 bp, 150 bp, 200 bp, 300 bp, 400 bp, and 500 bp).

YFP median fluorescence intensity is compared between cells with matching RFP and BFP transfection levels. n=3 samples per condition. n=3 samples per condition.

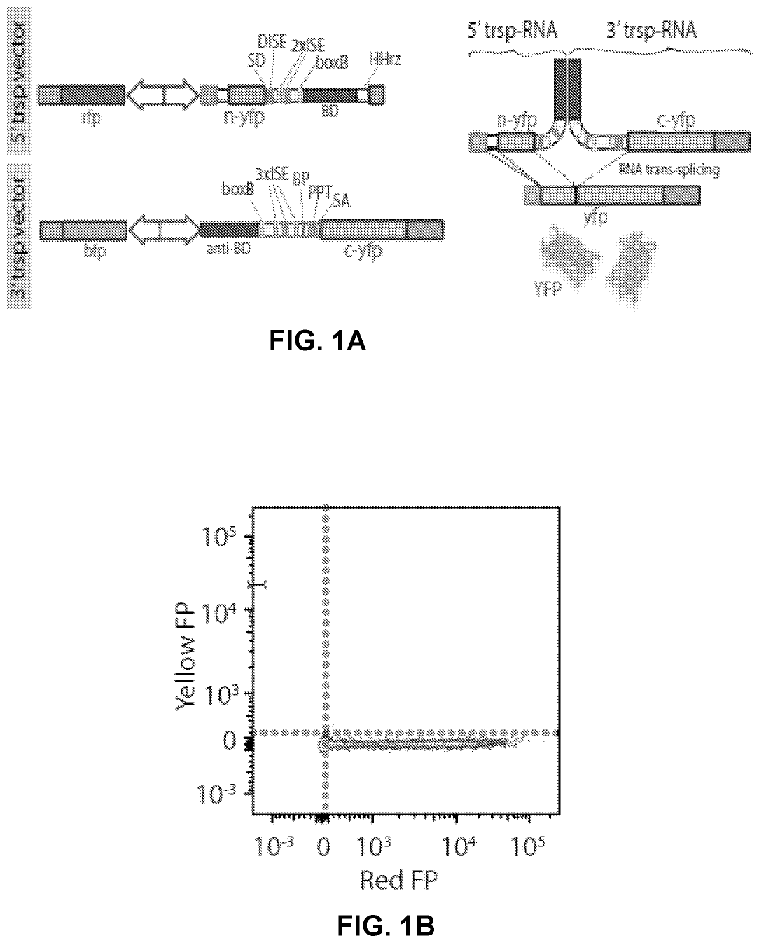

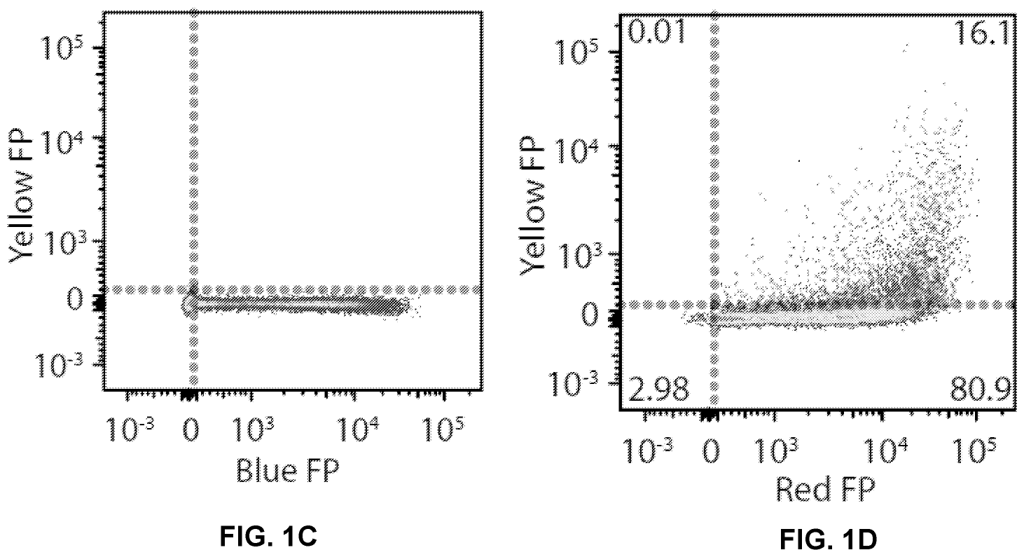

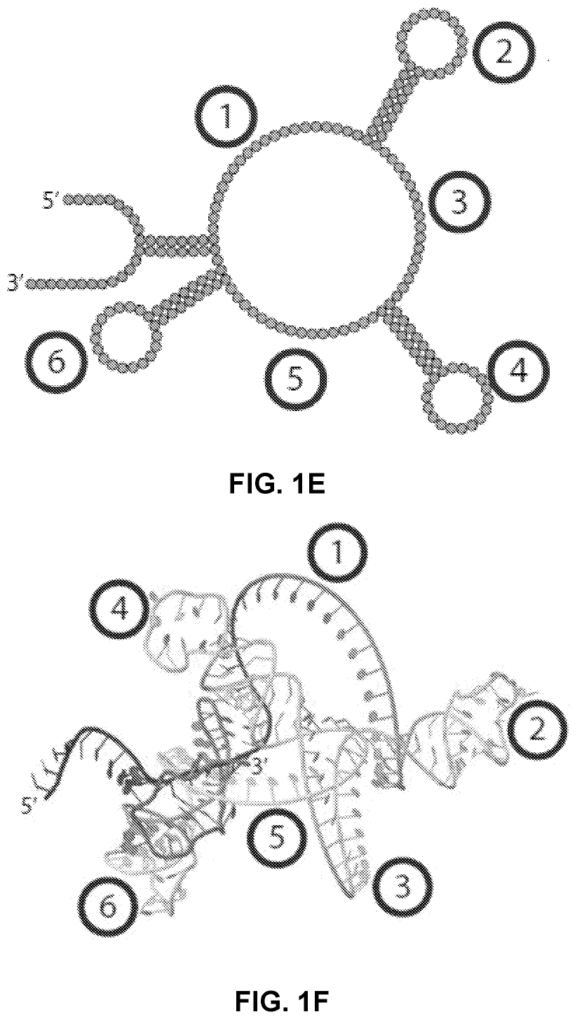

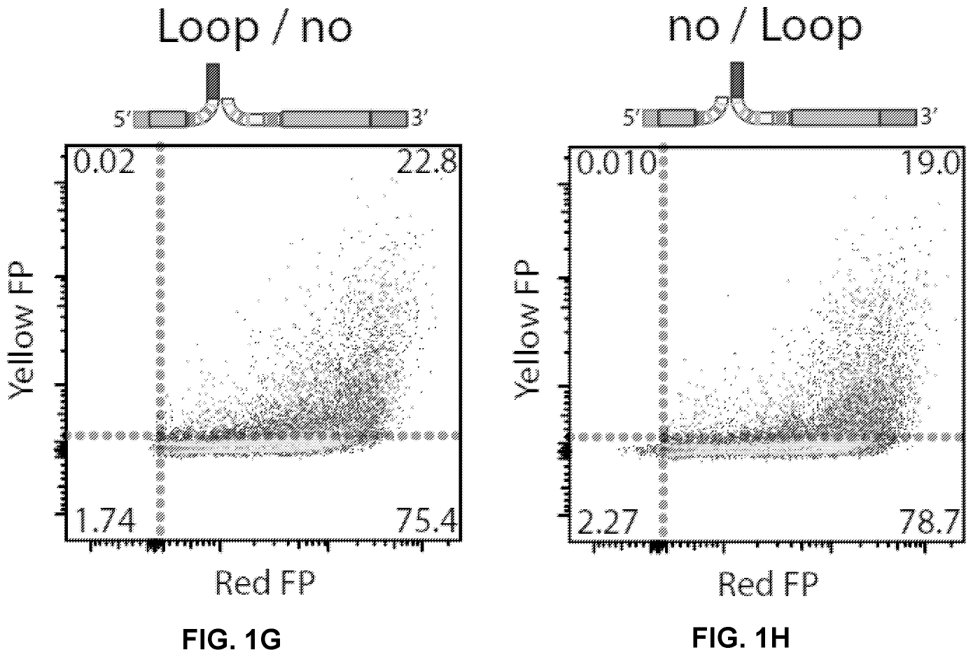

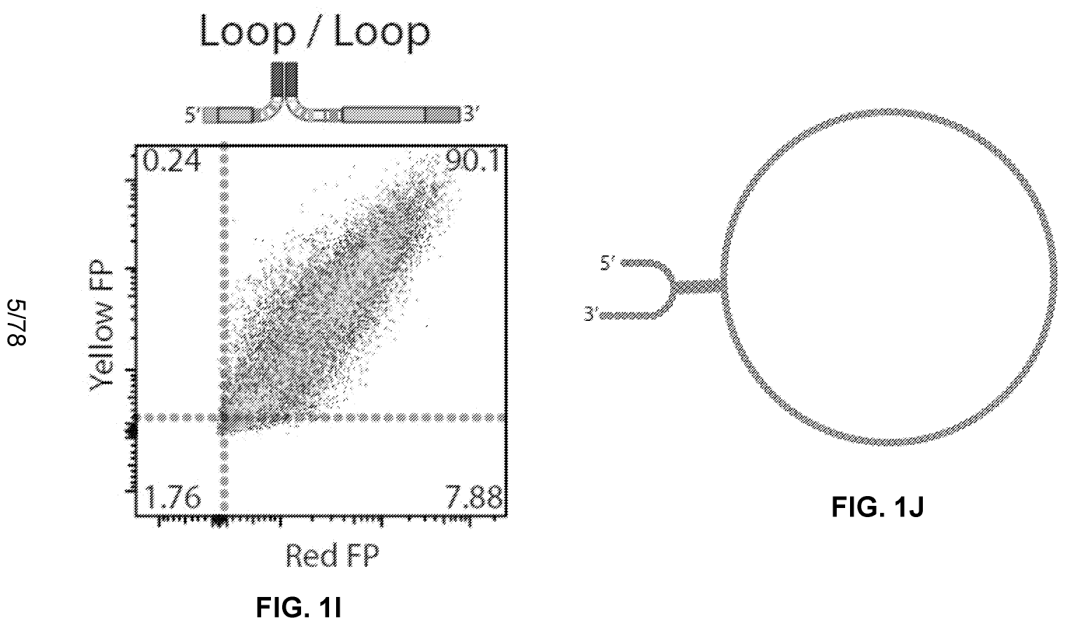

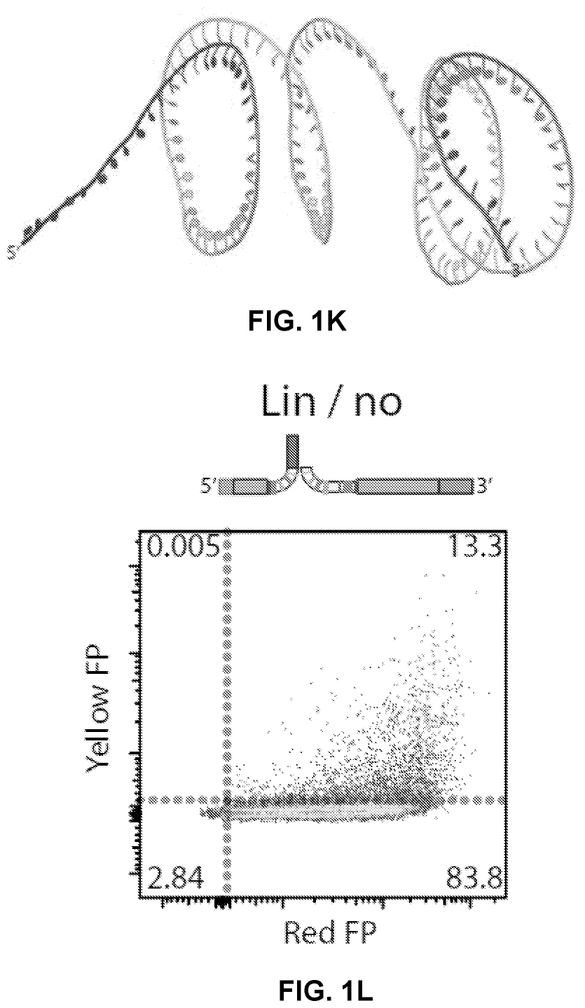

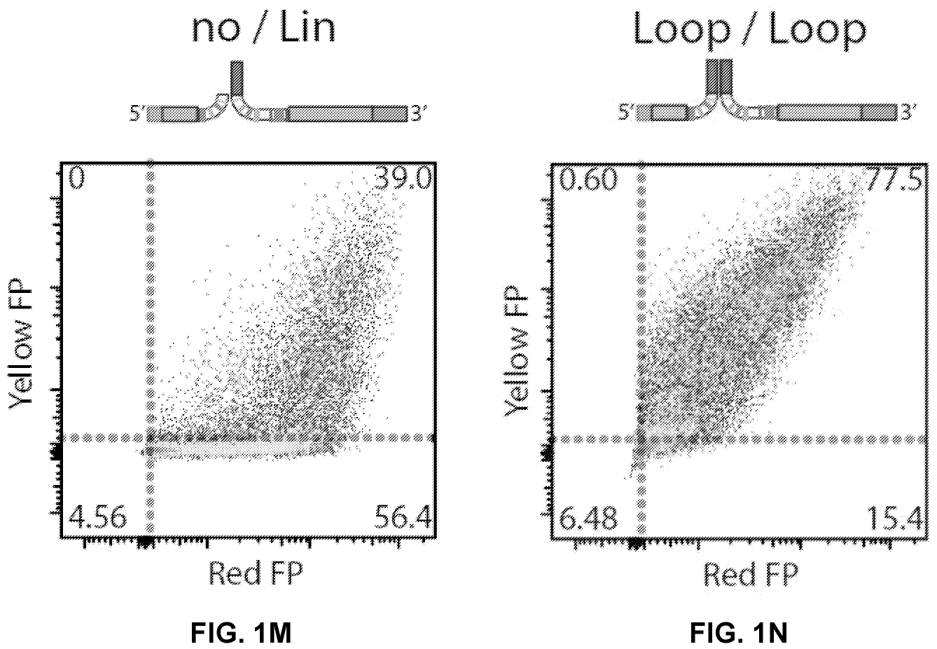

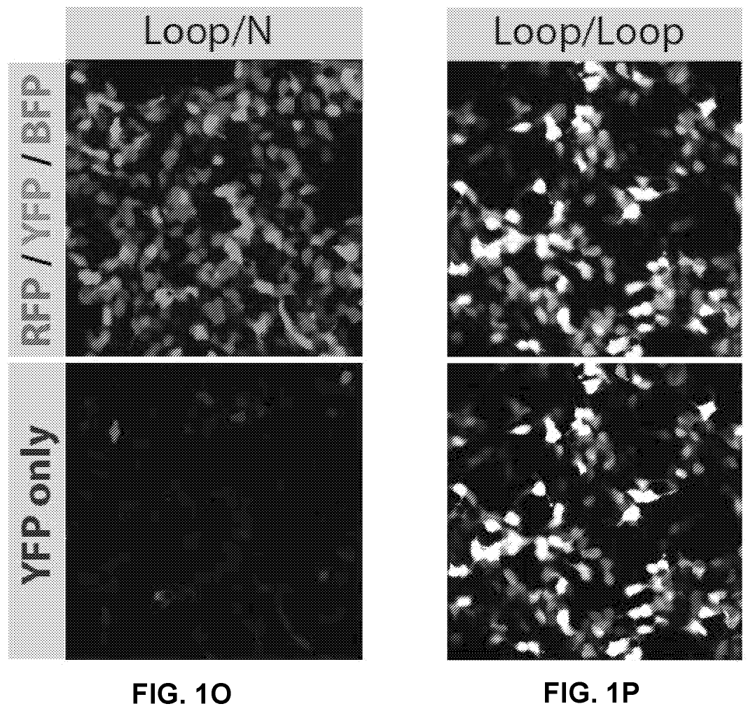

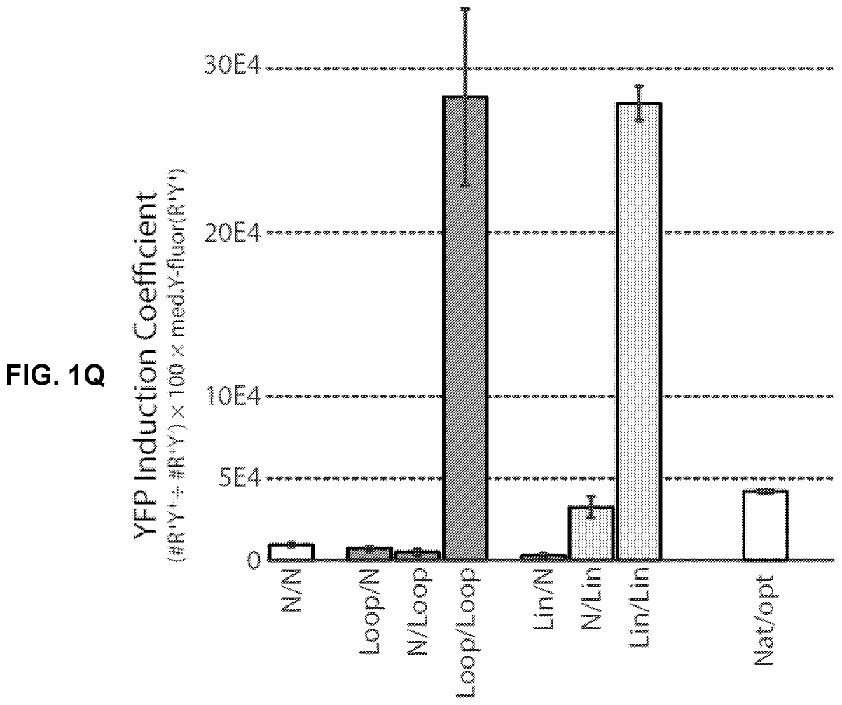

FIGS. 12A-12B show that inclusion of a splice enhancer into the synthetic intron increases the reconstitution efficiency. FIG. 12A is a schematic drawing of the 5′-N and 3′-C-terminal constructs used (SEQ ID NO: 1 and 2). (see FIG. 1A for abbreviations). FIG. 12B is a bar graph showing the resulting YFP fluorescence following transfection of SEQ ID NO: 1 and 2 into cells, or various truncations thereof. n=3 samples per condition.

FIGS. 13A-13D shows midline-crossing cortical neuron tracing by reconstitution of full-length flp recombinase (Flpo) from two fragments (SEQ ID NOS: 147 and 148). (A) Schematic representation of the 5′- and 3′-sequences used to reconstitute flpo (analogous to constructs in FIG. 12A) (B) Schematic representation of a flp-reporter mouse line injected with the N- and C-flpo encoding AAV virus injected into left and right regions of the cortex, respectively. (C and D) show neuronal cell body and axon labeling of cortical neurons that project to the contralateral hemisphere of the brain and therefore were infected by both the N-flpo and C-flpo viruses. Hoechst staining (nuclei) is shown for context.

FIGS. 14A-14D show expression of oversized cargo (i.e. proteins encoded by long RNAs) in cell culture and in vivo in the mouse primary motor cortex. (A) Schematic representation of the 5′- and 3′-sequences used to reconstitute YFP, which include long stuffer sequences (uninterrupted open reading frames; SEQ ID NOS: 22 and 23, respectively). (B) Quantitative real-time PCR analysis of reconstitution efficiency of the oversize YFP constructs in HEK 293t cells. N=3 per condition. (C) Reconstituted YFP protein expression from full-length oversized YFP expression and split-REJ expression assessed by flow cytometry of transiently transfected HEK 293t cells. Median yellow fluorescence intensity is compared between cell populations with equal transfection control (blue and red) fluorescence for the different conditions. Y-axis shows median yellow fluorescence intensity [a.u.]. N=3 per condition. (D) Schematic of injections into mouse primary motor cortex, and images of brain tissue 10 days following injection, showing successful reconstitution of a long (2401 aa) YFP protein in vivo.

FIGS. 15A-15C show efficient reconstitution of full-length human coagulation factor VIII (FVIII) with N-terminal HA tag (substituting the N-terminal signal peptide) (2317 aa). (A) Schematic representation of the 5′- and 3′-sequences used to reconstitute FVIII (SEQ ID NOS: 24 and 25, respectively). (B) PCR amplification of the junction. (C) Western blot showing expression of FVIII. Lanes 1-3: expression of full-length FVIII (290 kDa band shows full length, unprocessed FVIII). Lanes 4-6: expression of reconstituted FVIII (band at 290 kDa shows successfully reconstituted FVIII). Lanes 7 and 8: expression of the N-terminus only shows absence of full-length FVIII band at 290 kDa. For all lanes: Expected proteolytic processing products are observed ranging from −75 kDa to −210 kDa. FVIII is probed for using a mouse anti-HA primary antibody. All lanes were loaded with 5 micrograms of cleared cell protein extract. GAPDH (rabbit anti-GAPDH) is probed for as loading control.

FIGS. 16A-16F show efficient reconstitution of full-length human Abca4 with C-terminal FLAG-tag (2300 aa). (A) Schematic representation of the 5′- and 3′-sequences used to reconstitute Abca4 (SEQ ID NOS: 20 and 21, respectively), and a Sanger sequencing trace across the junction. (B) PCR amplification of the junction. (C) Schematic representation of the probes used to assay recombination of the 5′- and 3′-fragments. (D) PCR quantification of reconstitution efficiency after two days of expression in HEK 293t cells. N=2 per condition. (E) Western blot showing expression of Abca4. Lanes 1-3: expression of full-length Abca4 (˜260 kDa band shows full length Abca4). Lanes 4-6: expression of reconstituted Abca4 (band at 260 kDa shows successfully reconstituted Abca4). Lanes 7 and 8: no transfection control (i.e., HEK 293t lysate only) shows absence of any signal. Abca4 is probed for using a mouse anti-FLAG primary antibody. All lanes were loaded with 5 micrograms of cleared cell protein extract. GAPDH (rabbit anti-GAPDH) is probed for as loading control. (F) Quantification of the western blot in (E) normalized for differential BFP concentration. Data is shown as normalized to the average of full-length expression control.

FIGS. 17A and 17B provide (A) HIV-1 based kissing loop dimerization domain (N-fragment, SEQ ID NO: 139, C-fragment SEQ ID NO: 140); and (B) HIV-2 based kissing loop dimerization domain (N-fragment, SEQ ID NO: 141, C-fragment SEQ ID NO: 142).

FIGS. 18A-18C show efficient reconstitution of full-length murine Otof with C-terminal FLAG-tag (2019 aa). The DNA sequences of the 5′ and 3′ molecules used are shown in SEQ ID NOS: 155 and 156. (A) Western blot showing expression of Otof. Lanes 1-3: expression of full-length Otof (˜250 kDa band shows full length Otof). Lanes 4-6: expression of reconstituted Otof (band at 250k Da shows successfully reconstituted Otof). Lane 7: no transfection control (i.e., HEK 293t lysate only) shows absence of any signal. Otof is probed for using a mouse anti-FLAG primary antibody. All lanes were loaded with 5 micrograms of cleared cell protein extract. GAPDH (rabbit anti-GAPDH) is probed for as loading control. (B) Raw quantification of the western blot and (C) normalized for differential BFP concentration. Data is shown as normalized to the average of full-length expression control.

FIGS. 19A-19C show efficient reconstitution of full-length human Myo7a with C-terminal FLAG-tag (2243 aa). The DNA sequences of the 5′ and 3′ molecules used are shown in SEQ ID NOS: 157 and 158. (A) Western blot showing expression of Myo7a. Lanes 1-3: expression of full-length Myo7a (˜270 kDa band shows full length Myo7a). Lanes 4-6: expression of reconstituted Myo7a (band at 270 k Da shows successfully reconstituted Myo7a). Lane 7: no transfection control (i.e., HEK 293t lysate only) shows absence of any signal. Myo7a is probed for using a mouse anti-FLAG primary antibody. All lanes were loaded with 5 micrograms of cleared cell protein extract. GAPDH (rabbit anti-GAPDH) is probed for as loading control. (B) Raw quantification of the western blot and (C) normalized for differential BFP concentration. Data is shown as normalized to the average of full-length expression control.

FIGS. 20A-20D show efficient reconstitution of full-length DCas9-VPR (1951 aa). The DNA sequences of the 5′ and 3′ molecules used are shown in SEQ ID NOS: 159 and 160. (A) Western blot showing expression of DCas9-VPR. Lanes 1-3: expression of full-length DCas9-VPR (˜250 kDa band shows full length DCas9-VPR). Lanes 4-6: expression of reconstituted DCas9-VPR (band at 250 kDa shows successfully reconstituted DCas9-VPR). Lane 7: no transfection control (i.e., HEK 293t lysate only) shows absence of any signal. DCas9-VPR is probed for using a mouse anti-Cas9 primary antibody. All lanes were loaded with 5 micrograms of cleared cell protein extract. GAPDH (rabbit anti-GAPDH) is probed for as loading control. (B) Raw quantification of the western blot and (C) normalized for differential BFP concentration. Data is shown as normalized to the average of full-length expression control. (D) Example of transcriptional activation of a YFP expressing plasmid in HEK 293t cells. Full-length (upper panels) or two-way split REJ-dual dCas9-VPR (lower panels) is transiently transfected together with non-targeting guide RNA (left panels) or UAS-targeting guide RNA (right panels) expressing plasmids. All cells are also transfected with a UAS-YFP plasmid that is transcriptionally inactive until dCas9-VPR is targeted to the upstream region of a minimal promoter which results in expression of yellow fluorescent protein. Red fluorescent protein (RFP) is expressed with the N-terminal fragment of dCas9-VPR, Blue fluorescent protein (BFP) is expressed with the full-length dCas9-VPR or the C-terminal fragment of dCas9-VPR, respectively.

RFP and BFP serve as transfection control. Upon expression of both full-length as well as two-way split dCas9-VPR paired with a UAS-targeting guide RNA, yellow fluorescent protein expression is observed, confirming functionality of the reconstituted full-length protein.

FIGS. 21A-21D show efficient reconstitution of full-length humanized Prime Editor (2118 aa). The DNA sequences of the 5′ and 3′ molecules used are shown in SEQ ID NOS: 161 and 162. (A) Western blot showing expression of Prime Editor. Lanes 1-3: expression of full-length Prime Editor (˜260 kDa band shows full length Prime Editor). Lanes 4-6: expression of reconstituted Prime Editor (band at 260k Da shows successfully reconstituted Prime Editor). Lane 7: no transfection control (i.e., HEK 293tlysate only) shows absence of any signal. Prime Editor is probed for using a mouse anti-Cas9 primary antibody. All lanes were loaded with 5 micrograms of cleared cell protein extract. GAPDH (rabbit anti-GAPDH) is probed for as loading control. (B) Raw quantification of the western blot and (C) normalized for differential BFP concentration. Data is shown as normalized to the average of full-length expression control. (D) Shows Prime Editor induced G to T transversion mutations induced in the FANCF and the VEGFA3 loci of HEK293t cells. The top panel shows the sequence context for the FANCF and VEGFA3 loci respectively.

The grey arrow indicates the sequence targeted by the prime editor guide RNA (pegRNA). The protospacer adjacent motif (PAM) is indicated with a grey box. The G that is targeted for transversion to T is highlighted in the sequence. Genomic loci are sequenced using Sanger sequence in three conditions. The top panel shows a representative sanger trace for unedited wild type condition. The second from the top panel shows a representative sanger trace that represents the full-length expressed prime editor construct. The area highlighted with the black box shows the appearance of a T band in the sanger sequence, indicative of successful incorporation of the edit in a portion of the cells. The lowest panels show representative sanger traces for cells edited with a two-way split reconstituted prime editor. The appearance of a T trace (black box) demonstrates functionality of the prime editor when reconstituted from two fragments.

FIGS. 22A-22C show efficient reconstitution of full-length humanized Cytosine Base Editor (AncBE4) (1854 aa). The DNA sequences of the 5′ and 3′ molecules used are shown in SEQ ID NOS: 163 and 164. (A) Western blot showing expression of AncBE4. Lanes 1-3: expression of full-length AncBE4 (˜230 kDa band shows full length AncBE4). Lanes 4-6: expression of reconstituted AncBE4 (band at 230 k Da shows successfully reconstituted AncBE4). Lane 7: no transfection control (i.e., HEK 293t lysate only) shows absence of any signal. AncBE4 is probed for using a mouse anti-Cas9 primary antibody. All lanes were loaded with 5 micrograms of cleared cell protein extract. GAPDH (rabbit anti-GAPDH) is probed for as loading control. (B) Raw quantification of the western blot. Data is shown as normalized to the average of full-length expression control. (C) Shows AncBE4 induced C to T transition mutations induced in the EMX1 and the HEK site 3 loci of HEK293t cells. The top panel shows the sequence context for the EMX1 and HEK site 3 loci respectively. The grey arrow indicates the sequence targeted by the AncBE4 gRNA. The protospacer adjacent motif (PAM) is indicated with a grey box. The Cs that are targeted for transition to T are highlighted in the sequence. Genomic loci are sequenced using Sanger sequence in three conditions. The top panel shows a representative sanger trace for unedited wild type condition. The second from the top panel shows a representative sanger trace that represents the full-length expressed AncBE4 construct. The area highlighted with the black box shows the appearance of a T band in the sanger sequence, indicative of successful incorporation of the edit in a portion of the cells. The lowest panels show representative sanger traces for cells edited with a two-way split reconstituted AncBE4. The appearance of a T trace (black box) demonstrates functionality of the AncBE4 when reconstituted from two fragments.

FIGS. 23A-23C show efficient reconstitution of full-length humanized Adenine Base Editor (ABE8e) (1606 aa) (e.g., SEQ ID NOS: 225, 226). The DNA sequences of the 5′ and 3′ molecules used are shown in SEQ ID NOS: 165 and 166. (A) Western blot showing expression of ABE8e. Lanes 1-3: expression of full-length ABE8e (˜230 kDa band shows full length ABE8e). Lanes 4-6: expression of reconstituted ABE8e (band at 230 k Da shows successfully reconstituted ABE8e). Lane 7: no transfection control (i.e., HEK 293t lysate only) shows absence of any signal. ABE8e is probed for using a mouse anti-Cas9 primary antibody. All lanes were loaded with 5 micrograms of cleared cell protein extract. GAPDH (rabbit anti-GAPDH) is probed for as loading control. (B) Raw quantification of the western blot. Data is shown as normalized to the average of full-length expression control. (C) Shows ABE8e induced A to G transition mutations induced in the BCL11A and the HGB1/2 loci of HEK293t cells. The top panel shows the sequence context for the BCL11A and HGB1/2 loci respectively. The grey arrow indicates the sequence targeted by the ABE8e guide RNA (gRNA). The protospacer adjacent motif (PAM) is indicated with a grey box. The As that are targeted for transition to G are highlighted in the sequence. Genomic loci are sequenced using Sanger sequence in three conditions. The top panel shows a representative sanger trace for unedited wild type condition. The second from the top panel shows a representative sanger trace that represents the full-length expressed ABE8e construct. The area highlighted with the black box shows the appearance of a G band in the sanger sequence, indicative of successful incorporation of the edit in a portion of the cells. The lowest panels show representative sanger traces for cells edited with a two-way split reconstituted ABE8e. The appearance of a G trace (black box) demonstrates functionality of the ABE8e when reconstituted from two fragments.

FIG. 23D-G show efficient reconstitution of full-length humanized Adenine Base Editor (ABE8e) (1606 aa) to correct a premature stop codon in the mdx mouse model for Duchenne muscular dystrophy. (D) Schematic drawing of the experiment. The ABE8e base editor is split into two fragments, the coding DNA of which are individually packaged into two separate adeno-associated virus capsids. CRISPR gRNAs are designed to target the locus of the premature stop-codon such that the A to G conversion converts the TAA stop codon into a CAA codon. (E) A yellow fluorescent protein (YFP) is split into two coding fragments that are connected with a short stretch of the mdx coding sequence surrounding the premature stop codon. The first half of the YFP is non-fluorescent if the open reading frame is terminated by the premature TAA stop codon. Stop codon correction results in translation of the full YFP sequence which is rendered fluorescent (this construct is referred to as YFP-editing-reporter). The left panel shows absence of YFP fluorescence when HEK293T cells were transfected with the YFP-editing-reporter (which co-expresses a red fluorescent transfection control), and the N-terminal ABE8e vector, and the C-terminal ABE8e vector, and a non-targeting gRNA. The right panel shows expression of YFP fluorescence in a high percentage of cells that are cotransfected with the YFP-editing-reporter (which co-expresses a red fluorescent transfection control), and the N-terminal ABE8e vector, and the C-terminal ABE8e vector, and a mdx locus targeting gRNA. (F) Shows in vivo editing of the mdx premature stop codon resulting in expression of dystrophin in treated muscle. A male mdx mutant mouse was injected with a mixture of N- and C-terminal ABE8e packaged in adeno-associated virus 8 vectors (5E10 viral genomes per vector for a total of 1×10¹¹ viral genomes per muscle). The virus genomes contain two gRNA expression cassettes (composed of a RNA polymerase III promoter and the gRNA sequence) in each of the two genomes. The virus mix was injected intra muscularly into the tibialis anterior muscle. Top right panel shows dystrophin staining in a wild-type tibialis anterior muscle cross section for reference. The bottom left panel shows untreated tibialis anterior muscle tissue. The bottom right panel shows expression of dystrophin in a tibialis anterior muscle that was injected with the two adeno-associated viruses. (G) Shows dystrophin expression (top panel), ABE8e expression (middle panel), and a GAPDH loading control (bottom panel) of tibialis anterior muscle treated with the adeno-associated virus mix for expression of ABE8e. This illustrates the rescue of dystrophin expression using the ABE8e base editor.

FIGS. 24A-24C Influence of downstream intronic splicing enhancers (DISE) and intronic splicing enhancers (ISE) and acceptor sequences on the efficiency of RNA end joining. (A) Schematic depiction of screen setup. The 5′ fragment is an RNA molecule which is transcribed from a DNA construct using the human CMV promoter and enhancer. The RNA molecule produced contains a long stuffer open reading frame to simulate large cargo size. This stuffer sequence ends in a 2A self-cleaving peptide sequence and is followed by the coding region for the 5′ fragment of a Yellow Fluorescent Protein (n-yfp). The 5′ fragment of yfp ends in a splice donor site (SD). This splice donor site is followed by the 5′ intronic portion of the RNA end joining module. For the purpose of determining the impact of DISE and ISE sequences on RNA end joining reaction efficiency, the 5′ intronic portion is subdivided into three fragments: from 5′ to 3′: ds: downstream segment; m: mid intronic segment; dd: donor distal segment. The 5′ intronic portion is followed by a trimodal kissing loop RNA dimerization domain. The message is terminated with a short poly adenylation signal. The overall length of this 5′ RNA molecule is ˜4 kb to simulate a large cargo reconstitution scenario. The 3′ fragment is an RNA molecule which is transcribed from a DNA construct using the human CMV promoter and enhancer. The 3′ fragment starts with a trimodal kissing loop RNA dimerization domain that is complementary to the one on the 5′ fragment encoding RNA molecule. The dimerization domain is followed by the 3′ intronic portion of the RNA end joining module. This 3′ intronic portion is subdivided into three segments: ad: acceptor distal segment; m: mid-intronic segment; ap: acceptor proximal segment. The acceptor proximal segment contains variations of the branch point and polypyrimidine tracts which are both essential for the spliceosome mediated RNA joining reaction. The splice acceptor (SA) site is followed by the 3′ yfp coding sequence which is followed by a self-cleaving 2A sequence that is followed by a long stuffer open reading frame. The message is terminated by an SV40 poly adenylation signal. The overall length of the 3′ RNA molecule is ˜4 kb to simulate a large cargo reconstitution scenario. The association of the two RNA molecules (the 5′ fragment and the 3′ fragment) is mediated by the trimodal kissing loop RNA dimerization domain, the recruitment of the spliceosome and the RNA end joining reaction are mediated by the intronic segments. Successful RNA end joining results in reconstitution of the yfp open reading frame and subsequent translation of YFP. (B) Median YFP fluorescence intensity as determined by flow cytometry is shown for a number of intron configurations. In the first grouping (bars 1 to 9) a selection of potential downstream intronic splicing enhancer sequences were paired with a consensus splice donor site (GTAAGTATT in the DNA construct and GUAAGUAUU in the RNA sequence), shown in bars 1-8. These are compared to a consensus splice donor that is followed by scrambled sequence composed of equal parts of all four bases (ds9). In the second grouping, m1-m16 a selection of potential intronic splicing enhancers was compared to a scrambled sequence (m16). In the last grouping a selection of potential strong branch point, polypyrimidine tract, and splice acceptors were compared. The reference constructs were composed of scrambled sequence in all non-variable positions with a consensus donor followed by scrambled sequence in the ds position and a consensus splice acceptor sequence (where the whole polypyrimidine tract is composed of Ts in the DNA construct and Us in the RNA fragment respectively). (C) Listing of different DISE, ISE, and splice acceptor elements used.

FIGS. 25A-25B show the in vivo expression of a base editor in mouse muscle, and the correction of a dystrophin gene mutation using the disclosed methods. (A) Western blots showing expression of dystrophin, Cas9-ABE, and GAPDH in muscle extracts from wildtype (wt), untreated mdx-4 cv mice, and treated mdx-4cv mice. (B) Immunohistochemistry analysis of dystrophin expression in muscle in wildtype (wt), untreated mdx-4cv mice, and treated mdx-4cv mice.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on May 16, 2022, 299 KB, which is incorporated by reference herein. In the accompanying sequence listing:

SEQ ID NOS: 1 and 2 are N- and C-terminal sequences, respectively, used to express full-length YFP. SEQ ID NO: 1, CMV promoter nt 1 to 543, YFP coding sequence nt 544 to 1032, synthetic intron nt 1033 to 1436, and untranslated poly A region nt 1437 to 1491. SEQ ID NO: 2, CMV promoter nt 1 to 522, synthetic intron nt 523 to 904, YFP coding sequence nt 905 to 1141, and nt 1142 to 1302 is the untranslated poly A region.

SEQ ID NOS: 3 and 4 are 5′- and 3′-intronic sequences, respectively, that can be used to express a desired full-length protein, wherein a N-terminal portion of the full-length protein can be added at nt 1 of SEQ ID NO: 3, and C-terminal portion of the full-length protein can be added at nt 382 of SEQ ID NO: 4.

SEQ ID NOS: 5 and 6 are N- and C-terminal coding sequences, respectively, used to express full-length YFP.

SEQ ID NO: 7 is an exemplary synthetic intron dimerization domain (FIG. 10E).

SEQ ID NO: 8 is an exemplary synthetic intron without intronic splicing enhancers (FIG. 10F).

SEQ ID NO: 9 is an exemplary synthetic intron without intronic splicing enhancers (FIG. 10G).

SEQ ID NO: 10 is an exemplary synthetic intron without intronic splicing enhancers (FIG. 10H).

SEQ ID NO: 11 is an exemplary synthetic intron without binding domain (FIG. 10I).

SEQ ID NO: 12 is an exemplary synthetic intron with dimerization domain (FIG. 10I).

SEQ ID NO: 13 is an exemplary synthetic intron with dimerization domain (FIG. 10K).

SEQ ID NO: 14 is an exemplary synthetic intron without intronic splicing enhancers (FIG. 10L).

SEQ ID NO: 15 is an exemplary synthetic intron with DISE only (FIG. 10M).

SEQ ID NO: 16 is an exemplary synthetic intron without HHrz (FIG. 10N).

SEQ ID NO: 17 is an exemplary synthetic intron without intronic splicing enhancers (FIG. 10O).

SEQ ID NO: 18 is an exemplary U12 dependent intron with binding domain (FIG. 10P).

SEQ ID NO: 19 is an exemplary U12 dependent intron with binding domain (FIG. 10Q).

SEQ ID NOS: 20 and 21 are the N- and C-terminal DNA sequences, respectively, used to express RNAs (pre-mRNAs) resulting in full-length Abca4. In SEQ ID NO: 20, the sequence corresponding to the N-terminal Abca4 coding region is at nt 22 to 3702, and nt 3703 to 3912 is the synthetic intron, and 3921 to 3969 is the untranslated poly A region. SEQ ID NO: 20 also comprises a splice donor at nt 3703-3711, a Rat FGFR2 DISE at nt 3714-3737, a cTNT intronic splicing enhancer at nt 3747-3770, an M2 intronic splicing enhancer at nt 3782-3794, and a kissing loop dimerization domain at nt 3801-3975. In SEQ ID NO: 21, nt 1 to 228 is the synthetic intron, nt 229 to 3366 is the C-terminal Abca4 coding region, 3367 to 3447 is the FLAG epitope tag, and nt 3476 to 3607 is the untranslated poly A region (signal). SEQ ID NO: 21 also comprises a kissing loop dimerization domain at nt 3-114, an M2 intronic splicing enhancer at nt 121-133, a cTNT intronic splicing enhancer at nt 140-163, an M2 intronic splicing enhancer at nt 175-187, a Branch Point Motif at nt 194-201, a poly pyrimidine tract at nt 207-226, and a splice acceptor at nt 228.

SEQ ID NOS: 22 and 23 are the N- and C-terminal DNA sequences, respectively, used to express RNAs (pre-mRNAs) resulting in a long full-length YFP, wherein each includes splice enhancers. In SEQ ID NO: 22, the N-terminal YFP coding region is nt 22 to 3702, nt 3703 to 3912 is the synthetic intron, and 3921 to 3969 is the untranslated poly A region. SEQ ID NO: 22 also comprises a splice donor at nt 3703-3711, a Rat FGFR2 DISE at nt 3714-3737, a cTNT intronic splicing enhancer at nt 3747-3770, an M2 intronic splicing enhancer at 3782-3794, and a kissing loop dimerization domain at 3801-3975. In SEQ ID NO: 23, nt 1 to 225 is the synthetic intron, nt 226 to 3747 C-terminal YFP coding region, nt 3748 to 3912 is the untranslated poly A region. SEQ ID NO: 23 comprises a kissing loop dimerization domain at nt 3-114, an M2 intronic splicing enhancer at nt 118-130, a cTNT intronic splicing enhancer at nt 137-160, a M2 intronic splicing enhancer at nt 172-184, a Branch Point Motif at nt 191-198, a poly pyrimidine tract at nt 204-223, and a splice acceptor at nt 225.

SEQ ID NOS: 24 and 25 are the N- and C-terminal sequences, respectively, used to express RNAs (pre-mRNAs) resulting in full-length human Factor VIII. In SEQ ID NO: 24, N-terminal FVIII coding region with N-terminal HA epitope tag nt are at nt 22 to 3561, nt 3562 to 3771 is the synthetic intron, and nt 3780 to 3828 is the untranslated poly A region. SEQ ID NO: 24 also comprises a splice donor at nt 3562-3570, a Rat FGFR2 DISE at nt 3573-35%, a cTNT intronic splicing enhancer at nt 3606-3629, an M2 intronic splicing enhancer at nt 3641-3653, and a kissing loop dimerization domain at nt 3660-3834. In SEQ ID NO: 25, nt 1 to 225 is the synthetic intron, nt 226 to 3636 is the C-terminal FVIII coding region, and nt 3665 to 3797 is the untranslated poly A region. SEQ ID NO: 25 also comprises a splice donor at nt 3703-3711, a Rat FGFR2 DISE at nt 3714-3737, a cTNT intronic splicing enhancer at nt 3747-3770, an M2 intronic splicing enhancer at 3782-3794, and a kissing loop dimerization domain at nt 3801-3975.

SEQ ID NOS: 26-136 are exemplary splicing enhancers that can be used with the systems provided herein (e.g., 118, 120, 156 of FIG. 6A).

SEQ ID NOS: 137 and 138 are exemplary splice donor sequences.

SEQ ID NOS: 139 and 140 are the N- and C-fragment respectively, of an HIV-1 based kissing loop dimerization domain.

SEQ ID NOS: 141 and 142 are the N- and C-fragment, respectively, of an HIV-2 based kissing loop dimerization domain.

SEQ ID NO: 143 is an exemplary cryptic splice acceptor sequence.

SEQ ID NO: 144 is an exemplary branch point consensus sequence.

SEQ ID NOS: 145 and 146 are the N- and middle sequences, respectively, used to express a full-length YFP, along with SEQ ID NO: 2 (C-terminal fragment). In SEQ ID NO: 145, nt 1 to 543 is the CMV promoter sequence, nt 544 to 849 N-terminal YFP coding region, and nt 850 to 1305 is the synthetic intron. In SEQ ID NO: 146, nt 1 to 522 is the CMV promoter sequence, nt 523 to 901 is the synthetic intron, nt 902 to 1084 is the middle YFP coding region, and nt 1085 to 1543 is the untranslated poly A region.

SEQ ID NOS: 147 and 148 are the 5′ and 3′-synthetic sequences, respectively, used to express a full-length Flpo. In SEQ ID NO: 147, nt 1 to 540 is the CMV promoter sequence, nt 541 to 1112 N-terminal Flpo coding region, and nt 1113 to 1571 is the synthetic intron. In SEQ ID NO: 148, nt 1 to 522 is the CMV promoter sequence, nt 523 to 904 is the synthetic intron, nt 905 to 1604 is the C-terminal Flpo coding region, nt 1605 to 1765 is the untranslated poly A region.

SEQ ID NOS: 149 and 150 are exemplary hypodiverse sequences.

SEQ ID NOS: 151 and 152 are exemplary splice donor consensus sequences.

SEQ ID NO: 153 is an exemplary kissing loop based on the HIV-2 kissing loop dimerization domain (SEQ ID NOS: 141 and 142, FIG. 17B).

SEQ ID NO: 154 is an exemplary Kozak enhanced start codon.

SEQ ID NOS: 155 and 156 are exemplary constructs that can be used to express a murine Otof coding sequence in vivo. SEQ ID NO: 155 is used to produce the N-terminal Otof RNA. It comprises a human CMV enhancer and promoter at nt 1-522, a putative transcription start site at nt 523, and a poly adenylation signal at nt 4263-4311. It encodes the N-terminal Otof RNA elements as follows: 5′ untranslated region including Kozak sequence nt 523-546; 5′ Otoferlin coding sequence nt 547-4044; 5′ synthetic intron sequence nt 4045-4142; 5′ trimodal kissing loop dimerization domain nt 4143-4254; and linker at nt 4255-4262. SEQ ID NO: 155 is used to produce the C-terminal Otof RNA. It comprises a human CMV enhancer and promoter at nt 1-522, a putative transcription start site at nt 523, and a poly adenylation signal at nt 3335-3467. It encodes the C-terminal Otof RNA elements as follows: 3′ trimodal kissing loop dimerization domain nt 525-636; 3′ synthetic intron sequence nt 637-747; 3′ Otoferlin coding sequence nt 748-3225; C-terminal 3×Flag tag nt 3226-3306; and linker at nt 3307-3334.

SEQ ID NOS: 157 and 158 are exemplary constructs that can be used to express a human MYOSIN VIIA (Myo7a) coding sequence in vivo. SEQ ID NO: 157 is used to produce the N-terminal Myo7a RNA. It comprises a human CMV enhancer and promoter at nt 1-522, a putative transcription start site at nt 523, and poly adenylation signal at nt 4344-4392. It encodes the N-terminal Myo7A RNA elements as follows: 5′ untranslated region including Kozak sequence nt 523-543; 5′ Myo7a coding sequence nt 544-4125; 5′ synthetic intron sequence nt 4126-4223; 5′ trimodal kissing loop dimerization domain nt 4224-4335; and linker at nt 4336-4343. SEQ ID NO: 158 is used to produce the C-terminal Myo7a RNA. It comprises a human CMV enhancer and promoter at nt 1-522, a putative transcription start site at nt 523, and a poly adenylation signal at nt 3923-4055. It encodes the C-terminal Myo7a RNA elements as follows: 3′ trimodal kissing loop dimerization domain nt 525-636; 3′ synthetic intron sequence nt 637-747; 3′ Myo7a coding sequence nt 748-3813; C-terminal 3×Flag tag nt 3814-3894; and linker at nt 3895-3922.

SEQ ID NOS: 159 and 160 are exemplary constructs that can be used to express a full-length enzymatically dead Cas9 fused to a VPR transcriptional activator domain (dCas9-VPR) coding sequence in vivo. SEQ ID NO 159 is used to produce the N-terminal DCas9-VPR RNA. It comprises a human CMV enhancer and promoter at nt 1-522, a putative transcription start site at nt 523, and poly adenylation signal at nt 4112-4161. It encodes the N-terminal DCas9-VPR RNA elements as follows: 5′ untranslated region including Kozak sequence nt 523-543; 5′ DCas9-VPR coding sequence nt 544-3894; 5′ synthetic intron sequence nt 3895-3992; 5′ trimodal kissing loop dimerization domain nt 3993-4104; and linker nt 4105-4112. SEQ ID NO: 160 is used to produce the C-terminal DCas9-VPR RNA. It comprises a human CMV enhancer and promoter at nt 1-522, a putative transcription start site at nt 523, and poly adenylation signal zt nt 3278-3410. It encodes the C-terminal DCas9-VPR RNA elements as follows: 3′ trimodal kissing loop dimerization domain nt 525-636; 3′ synthetic intron sequence nt 637-747; 3′ DCas9-VPR coding sequence nt 748-3249; and linker at nt 3250-3277.

SEQ ID NOS: 161 and 162 are exemplary constructs that can be used to express a full-length humanized Cas9 Prime Editor (Prime Editor) coding sequence in vivo. SEQ ID NO: 161 encodes the N-terminal Prime Editor sequence as follows: Human CMV enhancer and promoter nt 1-522; putative transcription start site nt 523; 5′ untranslated region including Kozak sequence nt 523-543; 5′ Prime Editor coding sequence nt 544-3894; 5′ synthetic intron sequence nt 3895-3992; 5′ trimodal kissing loop dimerization domain nt 3993-4104; linker nt 4105-4112; poly adenylation signal nt 4112-4161. SEQ ID NO: 162 encodes the C-terminal Prime Editor sequence as follows: Human CMV enhancer and promoter nt 1-522; putative transcription start site nt 523; 3′ trimodal kissing loop dimerization domain nt 525-636; 3′ synthetic intron sequence nt 637-747; 3′ Prime Editor coding sequence nt 748-3750; linker nt 3751-3778; poly adenylation signal nt 3779-3911.

SEQ ID NOS: 163 and 164 are exemplary constructs that can be used to express a full-length humanized Cytosine Base Editor (AncBE4) coding sequence in vivo. SEQ ID NO: 163 encodes the N-terminal AncBE4 sequence as follows: Human CMV enhancer and promoter nt 1-522; putative transcription start site nt 523; 5′ untranslated region including Kozak sequence nt 523-540; 5′ AncBE4 coding sequence nt 541-2892; 5′ synthetic intron sequence nt 2893-2990; 5′ trimodal kissing loop dimerization domain nt 2991-3102; linker nt 3103-3110; poly adenylation signal nt 3111-3159. SEQ ID NO: 164 encodes the C-terminal AncBE4 sequence as follows: Human CMV enhancer and promoter nt 1-522; putative transcription start site nt 523; 3′ trimodal kissing loop dimerization domain nt 525-636; 3′ synthetic intron sequence nt 637-747; 3′ AncBE4 coding sequence nt 748-3957; linker nt 3958-3982; poly adenylation signal nt 3983-4115.

SEQ ID NOS: 165 and 166 are exemplary constructs that can be used to express a full-length humanized Adenine Base Editor (ABE8e) coding sequence in vivo. SEQ ID NO: 165 encodes the N-terminal ABE8e sequence as follows: Human CMV enhancer and promoter nt 1-522; putative transcription start site nt 523; 5′ untranslated region including Kozak sequence nt 523-540; 5′ ABE8e coding sequence nt 541-2706; 5′ synthetic intron sequence nt 2707-2804; 5′ trimodal kissing loop dimerization domain nt 2805-2916; linker nt 2917-2924; poly adenylation signal nt 2925-2973. SEQ ID NO: 166 encodes the C-terminal Abe8e sequence as follows: human CMV enhancer and promoter nt 1-522; putative transcription start site nt 523; 3′ trimodal kissing loop dimerization domain nt 525-636; 3′ synthetic intron sequence nt 637-747; 3′ ABE8e coding sequence nt 748-3399; linker nt 3400-3427; poly adenylation signal nt 3428-3560.

SEQ ID NO: 167 is an exemplary
kissing loop domain
(GATTTTTGACCTGCTCGATTGTCCACTGC
GAGCAGGTCTTTTGGAGTCGGGCGAGGCGG
AAGCCCGACTCCTTTTGGCATGCACGCTAG
CCGCGTCGTGCATGCCTTTTATC).
SEQ ID NO: 168 is an exemplary ISE, M2
(GGGTTATGGGACC).
SEQ ID NO: 169 is an exemplary ISE,
cTNT (GGCTGAGGGAAGGACTGTCCTGGG).
SEQ ID NO: 170 is an exemplary DISE,
Rat FGFR2 (CTCTTTCTTTCCATGGGTTGGCCT).

SEQ ID NOS: 171 and 172 are exemplary constructs that can be used to express a full-length YFP coding sequence. SEQ ID NO: 171 encodes the N-terminal YFP sequence as follows: Human CMV enhancer and promoter nt 1-522; putative transcription start site nt 523; 5′ untranslated region including Kozak sequence nt 523-543; 5′ Stuffer open reading frame nt 544-3654; self cleaving 2A sequence nt 3655-3729; 5′ yellow fluorescent protein segment nt 3730-4224; 5′ synthetic intron sequence (variable) nt 4225-4294; 5′ trimodal kissing loop dimerization domain (uppercase): 4295-4406; linker nt 4407-4414; poly adenylation signal nt 4415-4463. SEQ ID NO: 172 encodes the C-terminal YFP sequence as follows: Name: 3′ intron screening split YFP; Human CMV enhancer and promoter nt 1-522; Putative transcription start site nt 523; 3′ trimodal kissing loop dimerization domain nt 525-636; 3′ synthetic intron sequence (variable) nt 637-706; 3′ yfp coding sequence nt 707-940; self-cleaving 2A sequence nt 941-1006; 3′ stuffer open reading frame nt 1007-4228; linker nt 4229-4265; poly adenylation signal nt 4257-4388.

SEQ ID NOS: 173-180 are exemplary intronic splicing enhancer sequences.

SEQ ID NO: 181 is a scrambled sequence.

SEQ ID NOS: 182-1% are exemplary intronic splicing enhancer sequences.

SEQ ID NO: 197-198 are scrambled sequences.

SEQ ID NOS: 199-203 are exemplary intronic splicing enhancer sequences.

SEQ ID NO: 204 is a scrambled sequence.

SEQ ID NO: 205 is an exemplary
branch point sequence (TACTAACA).
SEQ ID NO: 206 is an exemplary
polyadenylation signal
AATAAAATATCTTTATTTTCATTACATCTG
TGTGTTGGTTTTTTGTGTG.

SEQ ID NOS: 207 and 208 are an exemplary Cas9 coding sequence and protein sequence, respectively.

SEQ ID NOS: 209 and 210 are an exemplary dCas9 coding sequence and protein sequence, respectively.

SEQ ID NOS: 211 and 212 are exemplary Cas13d nucleic acid and amino acid sequences, respectively.

SEQ ID NOS: 213 and 214 are exemplary Cas13d nucleic acid and amino acid sequences, respectively.

SEQ ID NOS: 215 and 216 are exemplary dead Cas13d (e.g., catalytically inactive) amino acid sequences.

SEQ ID NO: 217 is an exemplary native HEPN domain RXXXXH.

SEQ ID NOS: 218-221 are exemplary nuclear localization signal coding and protein sequences.

SEQ ID NO: 222 is an exemplary Cas13d protein sequence.

SEQ ID NO: 223 is an exemplary DR sequence that can be included in a gRNA sequence and used with the Cas13d protein of SEQ ID NO: 212 for RNA editing.

SEQ ID NO: 224 is an exemplary DR sequence that can be included in a gRNA sequence and used with the Cas13d protein of SEQ ID NO: for RNA editing.

SEQ ID NOS: 225 and 226 are exemplary constructs that can be used to express a full-length humanized Adenine Base Editor (ABE8e) coding sequence in vivo. SEQ ID NO: 225 encodes the N-terminal ABE8e sequence and comprises two gRNA expression cassettes as follows: nt 1-141 AAV2 Inverted Terminal Repeat; nt 159-255 CRISPR gRNA (reverse orientation), nt 256-504 human U6 RNA polymerase III promoter (reverse orientation), nt 512-1019 CMV promoter, nt 1034-1051 5′ untranslated region, nt 1052-3217 N-terminal ABE8e editor, nt 3218-3315 synthetic intron sequence, nt 3316-3427 Dimerization Domain, nt 3436-3485 poly adenylation signal, nt 3492-3706 H1 polymerase III promoter, nt 3707-3803 CRISPR gRNA, and nt 3825-3965 AAV2 Inverted Terminal Repeat. SEQ ID NO: 225 comprises SEQ ID NO: 165 and added gRNA expression cassettes. SEQ ID NO: 226 encodes the C-terminal ABE8e sequence and comprises two gRNA expression cassettes as follows: nt 1-141 AAV2 Inverted Terminal Repeat, nt 159-255 CRISPR gRNA (reverse orientation), nt 256-504 human U6 RNA polymerase III promoter (reverse orientation), nt 512-1019 CMV promoter, nt 1036-1147 Dimerization Domain, nt 1259-3910 3′ ABE8e coding sequence, nt 3939-4069 poly adenylation signal, nt 4078-4292 H1 polymerase III promoter, nt 4293-4389 CRISPR gRNA, and nt 4411-4551 AAV2 Inverted Terminal Repeat.

SEQ ID NO: 226 comprises SEQ ID NO: 166 and added gRNA expression cassettes.

### DETAILED DESCRIPTION

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Lid., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a nucleic acid molecule” means “including a nucleic acid molecule” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, and GenBank Accession Nos., are herein incorporated by reference in their entireties.

In order to facilitate review of the various embodiments of the disclosure, the following explanations of specific terms are provided: Administration: To provide or give a subject an agent, such as a therapeutic nucleic acid molecule provided herein (such as one encoding one or more portions of a nucleic acid editor protein, gRNA, or both), or other therapeutic agent, by any effective route. Exemplary routes of administration include, but are not limited to, injection (such as subcutaneous, intramuscular, intradermal, intraperitoneal, intrathecal, intratumoral, intraosseous, and intravenous), transdermal, intranasal, and inhalation routes. Administration can be systemic or local.

Aptamer: Nucleic acid molecules (such as DNA or RNA) that bind a specific target agent or molecule with high affinity and specificity. Aptamers can be used in the disclosed nucleic acid molecules as a dimerization domain. In one example, two aptamers can bind to each other, e.g., by standard basepairing, non-canonical base pair interactions, non-base pairing interactions, or a combination thereof, to mediate dimerization. In one example, aptamers allow RNA dimerization (and subsequent recombination) only in the presence of one or more targets recognized by the aptamer. Aptamers have been obtained through a combinatorial selection process called systematic evolution of ligands by exponential enrichment (SELEX) (see for example Ellington et al., Nature 1990, 346, 818-822; Tuerk and Gold Science 1990, 249, 505-510; Liu et al., Chem. Rev. 2009, 109, 1948-1998; Shamah et al., Acc. Chem. Res. 2008, 41, 130-138; Famulok, et al., Chem. Rev. 2007, 107, 3715-3743; Manimala et al., Recent Dev. Nucleic Acids Res. 2004, 1, 207-231; Famulok et al., Acc. Chem. Res. 2000, 33, 591-599; Hesselberth, et al., Rev. Mol. Biotech. 2000, 74, 15-25; Wilson et al., Annu. Rev. Biochem. 1999, 68, 611-647; Morris et al., Proc. Natl. Acad. Sci. U.S.A. 1998, 95, 2902-2907). In such a process, DNA or RNA molecules that are capable of binding a target molecule of interest are selected from a nucleic acid library consisting of 10′4-10” different sequences through iterative steps of selection, amplification and mutation. The affinity of the aptamers towards their targets can rival that of antibodies, with dissociation constants in as low as the picomolar range (Morris et al., Proc. Natl. Acad. Sci. U.S.A. 1998, 95, 2902-2907; Green et al., Biochemistry 1996, 35, 14413-14424).

Aptamers that are specific to a wide range of targets from small organic molecules such as adenosine, to proteins such as thrombin, and even viruses and cells have been identified (Liu et al., Chem. Rev. 2009, 109, 1948-1998; Lee et al., Nucleic Acids Res. 2004, 32, D95-D100; Navani and Li, Curr. Opin. Chem. Biol. 2006, 10, 272-281; Song et al., TrAC, Trends Anal. Chem. 2008, 27, 108-117). For example, aptamers are available that recognize metal ions such as Zn(II) (Ciesiolka et al., RNA 1: 538-550, 1995) and Ni(II) (Hofmann et al., RNA, 3:1289-1300, 1997); nucleotides such as adenosine triphosphate (ATP) (Huizenga and Szostak, Biochemistry, 34:656-665, 1995); and guanine (Kiga et al., Nucleic Acids Res., 26:1755-60, 1998); co-factors such as NAD (Kiga et al., Nucleic Acids Res., 26:1755-60, 1998) and flavin (Lauhon and Szostak, J. Am. Chem. Soc., 117:1246-57, 1995); antibiotics such as viomycin (Wallis et al., Chem. Biol. 4: 357-366, 1997) and streptomycin (Wallace and Schroeder, RNA 4:112-123, 1998); proteins such as HIV reverse transcriptase (Chaloin et al., Nucleic Acids Res., 30:4001-8, 2002) and hepatitis C virus RNA-dependent RNA polymerase (Biroccio et al., J. Virol. 76:3688-96, 2002); toxins such as cholera whole toxin and staphylococcal enterotoxin B (Bruno and Kiel, BioTechniques, 32: pp. 178-180 and 182-183, 2002); and bacterial spores such as the anthrax (Bruno and Kiel, Biosensors & Bioelectronics, 14:457-464, 1999).

Binding: An association between two substances or molecules, such as the hybridization of one nucleic acid molecule to another (or itself), such as between two dimerization domains, or the binding of an aptamer to its target. An oligonucleotide molecule binds or stably binds to another nucleic acid molecule if there are a sufficient number of complementary base pairs between the oligonucleotide molecule and the target nucleic acid to permit detection of that binding. In some examples, binding between nucleic acid molecules may occur directly. In some examples, binding between nucleic acid molecules may occur indirectly, e.g., through an intermediate molecule. Either direct binding or indirect binding may occur by standard base pairing, by non-canonical base pair interactions, by non-base pair interactions, or a combination thereof. Non-canonical base pair interactions may occur by any means of stabilization known to those of skill in the art, including but not limited to Hoogsteen base pairs and wobble base pairs. Non-base pair interactions can include binding through an intermediate molecule. In some examples, direct binding is between kissing loop dimerization domains. In some examples, direct binding is between hypodiverse dimerization domains. In some examples, direct binding is between aptamer regions. In some examples, direct binding between aptamer regions involves non-canonical base pair interactions. In some examples, direct binding between aptamer regions involves standard base pairing and non-canonical base pair interactions. In some examples, indirect binding occurs through a nucleic acid bridge. In some examples the nucleic acid bridge is an mRNA. A nonlimiting example of a nucleic acid bridge is depicted in FIG. 7B. In some examples, indirect binding occurs through an aptamer molecule. A nonlimiting example of indirect binding through an aptamer molecule is depicted in FIG. 7A. In some embodiments, indirect binding through an aptamer molecule involves non-base pair interactions between the aptamer molecule and the binding regions. In some embodiments, indirect binding through an aptamer molecule involves non-base pair interactions between the aptamer molecule and the binding regions, and base pairing interactions between the binding regions.

C-terminal portion: A region of a protein sequence that includes a contiguous stretch of amino acids that begins at or near the C-terminal residue of the protein. A C-terminal portion of the protein can be defined by a contiguous stretch of amino acids (e.g., a number of amino acid residues).

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

Cas9: An RNA-guided DNA endonuclease enzyme that that participates in the CRISPR-Cas immune defense against prokaryotic viruses. Cas9 has two active cutting sites (HNH and RuvC), one for each strand of the double helix. An exemplary native Cas9 sequence from S. pyogenes is shown in SEQ ID NO: 206.

Catalytically inactive (deactivated or dead) Cas9 (dCas9) proteins, which have reduced or abolished endonuclease activity but still binds to dsDNA, are also encompassed by this disclosure. In some examples, a dCas9 includes one or more mutations in the RuvC and HNH nuclease domains, such as one or more of the following point mutations: D10A, E762A, D839A, H840A, N854A, N863A, and D986A (e.g., based on numbering in SEQ ID NO: 208). An exemplary dCas9 sequence with D1OA and H840A substitutions is shown in SEQ ID NO: 210. In one example, the dCas9 protein has mutations D10A, H840A, D839A, and N863A (see, e.g., Esvelt et al, Nat. Meth. 10:1116-21, 2013).

Cas9 and dCas9 sequences are publicly available. For example, GenBank® Accession Nos. nucleotides 796693 . . . 800799 of CP012045.1 and nucleotides 1100046 . . . 1104152 of CP014139.1 disclose Cas9 nucleic acids, and GenBank® Accession Nos. NP_269215.1, AMA70685.1, and AKP81606.1 disclose Cas9 proteins. In some examples, a deactivated form of Cas9 (dCas9) is nuclease deficient (e.g., those shown in GenBank® Accession Nos. AKA60242.1 and KR011748.1). Activatable Cas9 proteins are provided in US Publication No. 2018-0073002-A1. In certain examples, a Cas9 or dCas9 used in the disclosed compositions or methods has at least 80% sequence identity, for example at least 85%, at least 90%, at least 95%, at least 96%, at least 97%, at least 98%, at least 99% sequence identity, or 100% to such sequences (such as SEQ ID NOS: 207, 208, 209, and 210), and retains the ability to be used in the disclosed compositions and methods (e.g., can be encoded by two or more separate molecules of the present disclosure, and subsequently recombined using the REJ methods provided herein).

Cas13d: An RNA-guided RNA endonuclease enzyme that can cut or bind RNA. Cas13d proteins specifically recognize direct repeat (DR) sequences present in gRNA having a particular secondary structure. Cas13d proteins include one or two HEPN domains. Native HEPN domains include the sequence RXXXXH (SEQ ID NO: 217), wherein X is any amino acid. A catalytically inactive, or “dead” Cas13d, which include mutated HEPN domain(s) and thus cannot cut RNA, but can process gRNA, are also encompassed by this disclosure (e.g., see SEQ ID NOS: 215 and 216). Such a dead Cas13d (dCas13d) can be targeted to cis-elements of pre-mRNA to manipulate alternative splicing.

Exemplary native and variant Cas13d protein sequences are provided in WO 2019/040664, U.S. Pat. Nos. 10,876,101 and 10,392,616 (all herein incorporated by reference in their entireties), as well as herein as SEQ ID NOS: 212, 214, 215, 216, and 222.

In one example, a full length (non-truncated) Cas13d protein is between 870-1080 amino acids long. In one example, the Cas13d protein is derived from a genome sequence of a bacterium from the Order Clostridiales or a metagenomic sequence. In one example, the corresponding DR sequence of a Cas13d protein is located at the 5′ end of the spacer sequence in the molecule that includes the Cas13d gRNA. In one example, the DR sequence in the Cas13d gRNA is truncated at the 5′ end relative to the DR sequence in the unprocessed Cas13d guide array transcript (such as truncated by at least 1 nt, at least 2 nt, at least 3 nt, at least 4 nt, at least 5 nt, such as 1-3 nt, 3-6 nt, 5-7 nt, or 5-10 nt). In one example, the DR sequence in the Cas13d gRNA is truncated by 5-7 nt at the 5′ end by the Cas13d protein. In one example, the Cas13d protein can cut a target RNA flanked at the 3′ end of the spacer-target duplex by any of a, U, G or C ribonucleotide and flanked at the 5′ end by any of a, U, G or C ribonucleotide.

In one example, a Cas13d protein has at least 80%, at least 85%, at least 90%, at least 91%, at least 92%, at least 95%, at least 96%, at least 97%, at least 98% or at least 99% sequence identity to SEQ ID NO: 212, 214, 215, 216, or 222. In one example, a Cas13d coding sequence encodes a Cas13d protein having at least 80%, at least 85%, at least 90%, at least 91%, at least 92%, at least 95%, at least 96%, at least 97%, at least 98% or at least 99% sequence identity to SEQ ID NO: 212, 214, 215, 216, or 222. In one example, a Cas13d coding sequence has at least 80%, at least 85%, at least 90%, at least 91%, at least 92%, at least 95%, at least %%, at least 97%, at least 98% or at least 99% sequence identity to SEQ ID NO: 211 or 213.

Complementarity: The ability of a nucleic acid to form hydrogen bond(s) with another nucleic acid sequence by either traditional Watson-Crick base pairing or other non-traditional types. A percent complementarity indicates the percentage of residues in a nucleic acid molecule (e.g., target DNA or RNA) which can form hydrogen bonds (e.g., Watson-Crick base pairing) with a second nucleic acid sequence, such as a gRNA (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. Thus, in some examples, a first dimerization domain and a second dimerization domain have perfect complementary to one another (e.g., 100%). In other examples, a first dimerization domain and a second dimerization domain ae substantially complementary to one another (e.g., at least 80%).

Contact: Placement in direct physical association, including a solid or a liquid form. Contacting can occur in vitro or ex vivo, for example, by adding a reagent to a sample (such as one containing cells), or in vivo by administering to a subject.

CRISPR/Cas system: A prokaryotic immune system that confers resistance to foreign genetic elements, such as plasmids and phages, and provides a form of acquired immunity. The system includes a Cas nuclease (e.g., Cas9, Cas13d) and a guide RNA (gRNA) that specifically binds to the target RNA or DNA and directs the Cas nuclease to a target site. The disclosed compositions, systems, and methods can be used to express a Cas nuclease from two or more different DNA molecules, which in some examples further encode one or more gRNAs to regulate gene expression, for example to increase or decrease expression of a target nucleic acid molecule, and/or to edit a sequence of a target nucleic acid molecule (for example to repair one or more mutations associated with a disease, such as a substitution, insertion or deletion).

Dead guide RNA (dgRNA): A guide RNA (gRNA) that can guide wild-type Cas nuclease (e.g., Cas9) to a target nucleic acid, but does not induce double strand DNA breaks. The shortened gRNAs contain shortened targeting sequences of about 14 to 15 nucleotides, whereas non-dead gRNAs contain targeting sequences of about 20 nucleotides. dgRNAs are further described, for example, in Dahlman et al. (2015) Nat. Biotechnol. 33:1159-1161; Kiani et al. (2015) Nat. Methods, 12:1051-1054; and Hsin-Kai Liao et al. (2017) Cell, 171:1495-1507, all herein incorporated by reference in their entirety. In some examples, the dgRNA is an RNA molecule (for example, when expressed in a cell). In some examples, the dgRNA is encoded by a DNA molecule (for example, when in a vector, such as a viral vector).

DNA Editing: A type of genetic engineering in which a DNA molecule (or nucleotides of the DNA) is inserted, deleted or replaced in a cell or organism using a nucleases (such as Cas9, Cas13d or dead versions thereof), which create site-specific strand breaks at desired locations in the DNA. The induced breaks are repaired resulting in targeted mutations or repairs. CRISPR/Cas methods, for example using the REJ systems provided herein to express a Cas nuclease, can be used to edit the sequence of one or more target DNAs, such as one associated with cancer (e.g., breast cancer, colon cancer, lung cancer, prostate cancer, melanoma), infectious disease (such as HIV, hepatitis, HPV, and West Nile virus), or neurodegenerative disorder (e.g., Huntington's disease or ALS). For example, DNA editing can be used to treat a disease or viral infection.

DNA insertion site: A site of the DNA that is targeted for, or has undergone, insertion of an exogenous polynucleotide. The disclosed methods include use of a nucleic acid editor expressed from two or more nucleic acid molecules provided herein, which can be used to target a DNA for manipulation at a DNA insertion site.

Downregulated or knocked down: When used in reference to the expression of a molecule, such as a target nucleic acid or protein, refers to any process which results in a decrease in production of the target nucleic acid or protein, but in some examples not complete elimination of the target RNA product or target nucleic acid function. In one example, downregulation or knock down does not result in complete elimination of detectable target nucleic acid/protein expression or activity. In some examples, downregulation or knock down of a target nucleic acid includes processes that decrease translation of the target RNA and thus can decrease the presence of corresponding proteins. The disclosed system can be used to downregulate any target nucleic acid/protein of interest.

Downregulation or knock down includes any detectable decrease in the target nucleic acid/protein. In certain examples, detectable target nucleic acid/protein in a cell or cell free system decreases by at least 10%, at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such an amount of target nucleic acid/protein detected in a corresponding untreated cell or sample). In one example, a control is a relative amount of expression in a normal cell (e.g., a non-recombinant cell that does not include a nucleic acid molecule for RNA recombination provided herein).

Effective amount: The amount of an agent (such as a system providing multiple vectors, each encoding a different portion of a nucleic acid editing protein, such as a Cas9 or Cas13d protein, for example in combination with an effective amount of one or more gRNAs that can hybridize to the nucleic acid target) that is sufficient to effect beneficial or desired results. An effective amount also can refer to an amount of correctly joined RNA or nucleic acid editing protein produced that is sufficient to effect beneficial or desired results, for example in combination with one or more gRNAs that can hybridize to the nucleic acid target.

An effective amount (also referred to as a therapeutically effective amount) may vary depending upon one or more of: the subject and disease condition being treated, the weight and age of the subject, the severity of the disease condition, the manner of administration and the like, which can be determined by one of ordinary skill in the art. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In one embodiment, an “effective amount” of two or more synthetic nucleic acid molecules provided herein, sufficient to treat a disease, such as a genetic disease or cancer. In one embodiment, an “effective amount” of two or more synthetic nucleic acid molecules provided herein is amount sufficient to increase the survival time of a treated patient, for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase the survival time of a treated patient, for example by at least 6 months, at least 9 months, at least 1 year, at least 1.5 years, at least 2 years, at least 2.5 years, at least 3 years, at least 4 years, at least 5 years, at least 10 years, at least 12 years, at least 15 years, or at least 20 years (as compared to no administration of the two or more synthetic nucleic acid molecules provided herein). In one embodiment, an “effective amount” of two or more synthetic nucleic acid molecules provided herein is an amount sufficient to increase mobility of a treated patient (such as a DMD patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase mobility of a treated patient (such as a DMD patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase cognitive ability of a treated patient (such as a DMD patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase respiratory function of a treated patient (such as a DMD patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase blood clotting of a treated patient (such as a hemophilia patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase vision of a treated patient (such as a Usher or Stargardt patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein). In one embodiment, an “effective amount” of two or more synthetic nucleic acid molecules provided herein is an amount sufficient to increase hearing of a treated patient (such as a Usher patient), for example by at least 10%, at least 20%, at least 25%, at least 50%, at least 70%, at least 75%, at least 80%, at least 90%, at least 95%, at least 99%, at least 100%, at least 200%, at least 300%, at least 400%, at least 500%, or at least 600% (as compared to no administration of the two or more synthetic nucleic acid molecules provided herein).

In one embodiment, an “effective amount” of two or more synthetic nucleic acid molecules provided herein is an amount sufficient to reduce calf muscle size of a treated DMD patient, for example by at least 10%, at least 20%, at least 25%, at least 50%, at least 70%, at least 75%, at least 80%, at least 90%, or at least 95% (as compared to no administration of the two or more synthetic nucleic acid molecules provided herein). In one embodiment, an “effective amount” of two or more synthetic nucleic acid molecules provided herein is an amount sufficient to reduce cardiomyopathy muscle size of a treated DMD patient, for example by at least 10%, at least 20%, at least 25%, at least 50%, at least 70%, at least 75%, at least 80%, at least 90%, or at least 95% (as compared to no administration of the two or more synthetic nucleic acid molecules provided herein). In some examples, combinations of these effects are achieved.

Guide RNA (gRNA): A synthetic nucleic acid sequence used to direct a Cas nuclease (or dead Cas nuclease) protein to a target nucleic acid sequence, such as a target DNA (e.g., genomic sequence) or target RNA sequence. gRNA molecules include, whether as part of a single nucleic acid molecule or divided into two or more nucleic acid molecules, (1) a portion with sequence complementarity to the target nucleic acid (such as at least 80%, at least 90%, at least 95%, or 100% sequence complementarity, and (2) a portion with secondary structure that binds to the Cas nuclease. Thus, one can change the target nucleic acid of the Cas protein by simply changing the target sequence present in the gRNA (See CRISPR-Cas9 Structures and Mechanisms. Fuguo Jiang and Jennifer A. Doudna, Annual Review of Biophysics, 46:1, 505-529 (2017)). In some examples, the gRNA is an RNA molecule (for example, when expressed in a cell). In some examples, a gRNA is encoded by a DNA molecule (for example, when part of a vector, such as a viral vector). A gRNA can include modified bases or chemical modifications (e.g., see Latorre et al., Angewandte Chemie 55:3548-50, 2016).

In some examples, a gRNA includes two or more MS2-binding loop sequences, which can be modified from the native MS2-binding loop sequence to increase GC content and/or shorten repetitive content. In some examples, the gRNA is modified to increase GC content and/or shorten repetitive content.

In some examples, the gRNA is a dead guide RNA (dgRNA). Increasing GC content and/or shortening the repetitive content of the gRNA can be used to convert an gRNA into a dgRNA, that is, a guide nucleic acid molecule that can direct a Cas nuclease to a target nucleic acid sequence, but does not induce a DNA double strand break (or RNA single strand break).

In one example, a gRNA directs a Cas DNA nuclease (such as Cas9) to a target DNA. In one such example, the gRNA includes from 5′ to 3′ (1) a CRISPR RNA (crRNA) region that includes a sequence designed to hybridize to a target DNA sequence (and in some examples edit the target DNA sequence) and a region that hybridizes with trans-activating crRNA (tracrRNA), and (2) a scaffold sequence (tracrRNA) necessary for Cas-binding. Thus, a gRNA can combine a crRNA and a tracrRNA into a single RNA transcript (referred to in the art as a single guide RNA, sgRNA; for simplicity, encompassed by the term “gRNA” herein). In this example, a region of the crRNA hybridizes with tracrRNA to form a unique dual-RNA hybrid structure that binds Cas endonuclease proteins and guides the protein to a target DNA molecule. In some examples, the cRNA and tracrRNA are two separate RNA molecules (e.g., 2 piece gRNA; for simplicity, also encompassed by the term “gRNA” herein). In some examples, a protospacer adjacent motif (PAM) immediately follows the site of the target DNA to be edited (e.g., Cas9 cleavage site about 3 nt upstream of PAM).

In another example, a gRNA directs a Cas RNA nuclease (such as Cas13d) to a target RNA. In one such example, the gRNA includes from 5′ to 3′ (1) a crRNA containing a direct repeat (DR) region and (2) a spacer, for example for Cas13a, Cas13c, and Cas 13d nucleases. In one example includes about 36 nt of DR followed by about 28-32 nt of spacer sequence. In another such example, the gRNA includes from 5′ to 3′ (1) a spacer and (2) a crRNA containing a DR region, for example for Cas13b nuclease. In some examples, the gRNA is processed (truncated/modified) by a Cas RNA nuclease or other RNases into the shorter “mature” form. The DR is the constant portion of the gRNA, containing secondary structure which facilitates interaction between the Cas RNA nuclease protein and the gRNA. The spacer portion is the variable portion of the gRNA, and includes a sequence designed to hybridize to a target RNA sequence (and in some examples edit the target RNA sequence). In some examples, the full length spacer is about 28-32 nt (such as 30-32 nt) long while the mature (processed) spacer is about 14-30 nt.

Hybridization: Hybridization of a nucleic acid occurs when two nucleic acid molecules undergo an amount of hydrogen bonding to each other. The stringency of hybridization can vary according to the environmental conditions surrounding the nucleic acids, the nature of the hybridization method, and the composition and length of the nucleic acids used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N Y, 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of nucleic acid is hybridized to its complementary strand.

Increase or Decrease: A statistically significant positive or negative change, respectively, in quantity from a control value (such as a value representing no therapeutic agent, such as no administration of the two or more synthetic nucleic acid molecules provided herein). An increase is a positive change, such as an increase at least 50%, at least 100%, at least 200%, at least 300%, at least 400% or at least 500% as compared to the control value. A decrease is a negative change, such as a decrease of at least 20%, at least 25%, at least 50%, at least 75%, at least 80%, at least 90%, at least 95%, at least 98%, at least 99%, or at least 100% decrease as compared to a control value. In some examples the decrease is less than 100%, such as a decrease of no more than 90%, no more than 95%, or no more than 99%.

Isolated: An “isolated” biological component (such as a nucleic acid molecule or a protein) has been substantially separated, produced apart from, or purified away from other biological components in the cell or tissue of an organism in which the component occurs, such as other cells (e.g., RBCs), chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

Kissing loop/kissing stem loop: An RNA structure that forms when bases between two hairpin loops form pair interactions. These intermolecular “kissing interactions” occur when the unpaired nucleotides in one hairpin loop, base pair with the unpaired nucleotides in another hairpin loop to form a stable interaction complex. See FIG. 9A for an example.

N-terminal portion: A region of a protein sequence that includes a contiguous stretch of amino acids that begins at the N-terminal residue of the protein. An N-terminal portion of the protein can be defined by a contiguous stretch of amino acids (e.g., a number of amino acid residues).

Non-naturally occurring, synthetic, or engineered: Terms used herein as interchangeably and indicate the involvement of the hand of man. The terms, when referring to nucleic acid molecules or polypeptides indicate that the nucleic acid molecule or the polypeptide is at least substantially free from at least one other component with which they are naturally associated in nature and as found in nature. In addition, the terms can indicate that the nucleic acid molecules or polypeptides have a sequence not found in nature.

Nucleic ad molecule: A deoxyribonucleotide (DNA) or ribonucleotide (RNA) polymer, which can include natural nucleotides/ribonucleotides and/or analogues of natural nucleotides/ribonucleotides that hybridize to nucleic acid molecules in a manner similar to naturally occurring nucleotides. A nucleic acid molecule can be a single stranded (ss) DNA or RNA molecule or a double stranded (ds) nucleic acid molecule. RNA or mRNA as used herein may refer to a pre-mRNA molecule, or a mature RNA transcript.

A pre-mRNA molecule comprises sequences to be removed by processing, e.g., intron sequences removed by splicing following binding of the dimerization domains described herein. Nucleic acid molecules described herein can be DNA molecules from which an RNA is transcribed from a promoter on the DNA, e.g., in the context of a DNA expression vector.

Operably linked: A first nucleic acid sequence is operably linked with a second nucleic acid sequence when the first nucleic acid sequence is placed in a functional relationship with the second nucleic acid sequence. For instance, a promoter sequence is operably linked to a nucleic acid sequence if the promoter affects the expression of the nucleic acid sequence, for example, the promoter effects transcription of a pre-mRNA, which when spliced may result in expression of a protein (such as a portion of a nucleic acid editing protein coding sequence).

Pharmaceutically acceptable carriers: The pharmaceutically acceptable carriers useful in this disclosure are conventional. Remington's Pharmaceutical Sciences, by E. W. Martin, Mack Publishing Co., Easton, PA, 15th Edition (1975), describes compositions and formulations suitable for pharmaceutical delivery of a therapeutic agent, such as a nucleic acid molecule disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide, peptide and protein: Refer to polymers of amino acids of any length. The polymer may be linear or branched, it may include modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. In one example, a protein is nucleic acid editing protein, such as Cas9, Cas13d, or a zinc finger nuclease. In one example, a protein is one associated with disease, such as a genetic disease (e.g., sees Table 1-4). In one example, a protein is a therapeutic protein, such as one used in the treatment of a disease, such as cancer. In one example a protein is at least 50 as in length, at least 100 aa in length, at least 500 aa in length, at least 1000 aa in length, at least 1500 aa in length, such as at least 2000 aa, at least 2500 aa, at least 3000 aa, or at least 5000 aa.

Polypyrimidine tract: A region of pre-messenger RNA (mRNA) that promotes the assembly of the spliceosome, the protein complex specialized for carrying out RNA splicing during the process of post-transcriptional modification. This tract can be primarily pyrimidine nucleotides, such as uracil, and in some examples is 15-20 base pairs long, located about 5-40 base pairs before the 3′ end of the intron to be spliced.

Promoter/Enhancer: An array of nucleic acid control sequences which direct transcription of a nucleic acid sequence. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. In some examples a promoter sequence+its corresponding coding sequence is larger than the capacity for an AAV. In some examples a promoter sequence of a target protein is at least 3500 nt, at least 4000 nt, at least 5000 nt, or even at least 6000 nt.

A “constitutive promoter” is a promoter that is continuously active and is not subject to regulation by external signals or molecules. In contrast, the activity of an “inducible promoter” is regulated by an external signal or molecule (for example, a transcription factor). Both constitutive and inducible promoters can be used in the methods and systems provided herein (see e.g., Bitter et al., Methods in Enzymology 153:516-544, 1987). A tissue-specific promoter can be used in the methods and systems provided herein, for example to direct expression primarily in a desired tissue or cell of interest, such as muscle, neuron, bone, skin, blood, specific organs (e.g., liver, pancreas), or particular cell types (e.g., lymphocytes). In some examples, a promoter used herein is endogenous to the target protein expressed. In some examples, a promoter used herein is exogenous to the target protein expressed.

Also included are promoter elements which are sufficient to render promoter-dependent gene expression controllable for cell-type specific, tissue-specific, or inducible by external signals or agents; such elements may be located in the 5′ or 3′ regions of the gene. Promoters produced by recombinant DNA or synthetic techniques can also be used to provide for transcription of the nucleic acid sequences.

Exemplary promoters that can be used with the methods and systems provided herein include, but are not limited to an SV40 promoter, cytomegalovirus (CMV) promoter (optionally with the CMV enhancer), a pol II promoter (e.g., U6 and H1 promoters), a pol II promoter (e.g., the retroviral Rous sarcoma virus (RSV) LTR promoter (optionally with the RSV enhancer), the dihydrofolate reductase promoter, the β-actin promoter, the phosphoglycerol kinase (PGK) promoter, and the EFIα promoter).

Recombinant: A recombinant nucleic acid molecule or protein sequence is one that has a sequence that is not naturally occurring or has a sequence that is made by an artificial combination of two otherwise separated segments of sequence (e.g., a viral vector that includes a portion of a nucleic acid editing proteincoding sequence, such as about a third, half, or two-thirds of a coding sequence). This artificial combination can be accomplished by, for example, chemical synthesis or the artificial manipulation of isolated segments of nucleic acids, such as by genetic engineering techniques. Similarly, a recombinant or transgenic cell is one that contains a recombinant nucleic acid molecule.

RNA Editing: A type of genetic engineering in which a RNA molecule (or ribonucleotides of the RNA) is inserted, deleted or replaced in a cell or organism using engineered nucleases (such as the Cas13d and dCas13d proteins), which create site-specific strand breaks at desired locations in the RNA. The induced breaks are repaired resulting in targeted mutations or repairs. CRISPR/Cas methods, for example using the REJ systems provided herein to express a Cas nuclease, can be used to edit the sequence of one or more target RNAs, such as one associated with cancer (e.g., breast cancer, colon cancer, lung cancer, prostate cancer, melanoma), infectious disease (such as HIV, hepatitis, HPV, and West Nile virus), or neurodegenerative disorder (e.g., Huntington's disease or ALS). For example, RNA editing can be used to treat a disease or viral infection.

RNA Insertion site: A site of the RNA that is targeted for, or has undergone, insertion of an exogenous polynucleotide or polyribonucleotide. The disclosed methods include use of a nucleic acid editor expressed from two or more nucleic acid molecules provided herein, which can be used to target a RNA for manipulation at an RNA insertion site.

Sequence identity: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Variants of a native protein or coding sequence (such as a DMD, factor 8, factor 9, or ABCA4 sequence) are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. These sequence identity ranges are provided for guidance only; it is possible that strongly significant homologs could be obtained that fall outside of the ranges provided.

Variants of the disclosed nucleic acid sequences (such as synthetic intron sequences and coding sequences) are typically characterized by possession of at least about 80%, at least 90%, at least 95%, at least 96%, at least 97%, at least 98% or at least 99% sequence identity counted over the full length alignment with the nucleic acid sequence using the NCBI Blast 2.0, gapped blastn set to default parameters. One of skill in the art will appreciate that these sequence identity ranges are provided for guidance only; it is possible that functional sequences could be obtained that fall outside of the ranges provided.

Subject: A mammal, for example a human. Mammals include, but are not limited to, murines, simians, humans, farm animals, sport animals, and pets. In one embodiment, the subject is a non-human mammalian subject, such as a monkey or other non-human primate, mouse, rat, rabbit, pig, goat, sheep, dolphin, dog, cat, horse, or cow. In some examples, the subject is a laboratory animal/organism, such as a mouse, rabbit, or rat. In some examples, the subject treated using the methods disclosed herein is a human.

In some examples, the subject has genetic disease, such as one listed in Tables 1-4, that can be treated using the methods disclosed herein. In some examples, the subject treated using the methods disclosed herein is a human subject having a genetic disease. In some examples, the subject treated using the methods disclosed herein is a human subject having cancer. In some examples, the subject treated using the methods disclosed herein is a human subject having an infection, such as a bacterial or viral infection.

Target nucleic acid: A nucleic acid molecule, such as a DNA or RNA sequence, such as a gene, having a sequence that is to be altered and/or whose expression is to be modulated. In some examples, a target nucleic acid molecule is one nucleic acid molecule, two or more portions of the same nucleic acid molecule (e.g., same RNA or gene), two or more different nucleic acid molecules (e.g., two different genes or two different RNAs), or two or more portions of the two or more different nucleic acid molecules. The target nucleic acid molecule can include one or more target editing sites, that is a regions of the target nucleic acid molecule (such as one or more nucleotides or ribonucleotides, such as at least 10, at least 15, at least 20, or at least 30 consecutive nucleotides or ribonucleotides of the target) to be altered, such as substituted, deleted, or where an insertion is to be made. In some examples, a target nucleic acid molecule is one whose expression is to be modulated, such as an increase or decrease in expression of the gene product (e.g., protein). In one example, a target nucleic acid molecule is a DNA, RNA, or gene whose activated expression is desired. In one example, a target nucleic acid molecule is a DNA, RNA, or gene whose reduced or abolished expression is desired. In one example, a target nucleic acid molecule is a DNA, RNA, or gene having one or more point mutations that results in disease (such as one listed in Tables 1-4). In some examples, a targeting sequence (for example of a gRNA) has complementarity to the target gene/nucleic acid. In other examples, a targeting sequence (for example of a gRNA) has complementarity to a promoter and/or regulatory element of a target nucleic acid molecule. In some examples, the target nucleic acid sequence is DNA, and is present immediately adjacent to a Protospacer Adjacent Motif (PAM). In some examples, the target nucleic acid sequence is unique as compared to other nucleic acid sequences in the cell.

Targeting sequence: The portion of a gRNA having complementarity with a target nucleic acid sequence. In some examples, the targeting sequence has complementarity to a promoter or regulatory element of a target nucleic acid whose activated or repressed expression is desired. In some examples, the targeting sequence of a gRNA is about 14-30 nt and has sufficient complementarity with a target nucleic acid sequence to hybridize with the target sequence and direct sequence-specific binding of a Cas nuclease to the target nucleic acid sequence. In some embodiments, the degree of complementarity between a targeting sequence of an gRNA and its corresponding target nucleic acid, when optimally aligned using a suitable alignment algorithm, is about or more than about 50%, 60%, 75%, 80%, 85%, 90%, 95%, 97.5%, 98%, 99%, or more. In some embodiments, the degree of complementarity is 100%. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

Therapeutic agent: Refers to one or more molecules or compounds that confer some beneficial effect upon administration to a subject. The disclosed synthetic nucleic acid molecules and systems provided herein are therapeutic agents. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

Transcriptional activator: A protein or protein domain that increases transcription of a nucleic acid molecule, such as a gene. Such proteins can be used in the compositions, systems and methods provided herein. Such proteins and proteins domains can have a DNA binding domain and a domain for activation of transcription. These activators can be introduced into the system through attachment to a Cas nuclease or gRNA. Examples of such activators include VP64, p65, myogenic differentiation 1 (MyoDI), heat shock transcription factor (HSF) 1, RTA, CBP, SET7/9, or any combination thereof (such as p65 and HSF1).

Transduced, Transformed and Traufected: A virus or vector “transduces” a cell when it transfers nucleic acid molecules into a cell. A cell is “transformed” or “transfected” by a nucleic acid transduced into the cell when the nucleic acid becomes stably replicated by the cell, either by incorporation of the nucleic acid into the cellular genome, or by episomal replication.

These terms encompasses all techniques by which a nucleic acid molecule can be introduced into such a cell, including transfection with viral vectors, transformation with plasmid vectors, and introduction of naked DNA by electroporation, lipofection, particle gun acceleration and other methods in the art. In some example the method is a chemical method (e.g., calcium-phosphate transfection), physical method (e.g., electroporation, microinjection, particle bombardment), fusion (e.g., liposomes), receptor-mediated endocytosis (e.g., DNA-protein complexes, viral envelope/capsid-DNA complexes) and biological infection by viruses such as recombinant viruses (Wolff, J. A., ed, Gene Therapeutics, Birkhauser, Boston, USA, 1994). Methods for the introduction of nucleic acid molecules into cells are known (e.g., see U.S. Pat. No. 6,110,743). These methods can be used to transduce a cell with the disclosed nucleic acid molecules.

Transgene: An exogenous gene, for example supplied by a vector, such as AAV. In one example, a transgene encodes a portion of a nucleic acid editing protein, such as about a third, half, or two-thirds of a nucleic acid editing protein, for example operably linked to a promoter sequence. In one example, a transgene includes a portion of a Cas nuclease coding sequence, such as about a third, half, or two-thirds of a Cas nucleasecoding sequence, for example operably linked to a promoter sequence.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, blood and other clinical tests, and the like. In some examples, treatment with the disclosed methods results in a decrease in the number or severity of symptoms associated with a genetic disease, such as increasing the survival time of a treated patient with the genetic disease.

In some examples, treatment with the disclosed methods results in a decrease in the number or severity of symptoms associated with DMD or other genetic disease, such as increasing survival, increasing the mobility (e.g., walking, climbing), improving cognitive ability, reducing calf muscle size, reduce cardiomyopathy, improving vision, improving hearing, improving blood clotting, or improve respiratory function. In some examples, combinations of these effects are achieved.

Tumor, neoplasla, malignancy or cancer: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Exemplary tumors, such as cancers, that can be treated with the disclosed methods and systems include solid tumors, such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, bepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, and lymphatic tumors (including B-cell and T-cell malignant lymphoma). In one example, the tumor is an adenocarcinoma.

The methods and systems can also be used to treat liquid tumors, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), lymphomas (such as Hodgkin's lymphoma and non-Hodgkin's lymphoma), and myelomas).

Upregulated: When used in reference to the expression of a molecule, such as a target nucleic acid/protein, refers to any process which results in an increase in production of the target nucleic acid/protein. In some examples, upregulation or activation of a target RNA includes processes that increase translation of the target RNA and thus can increase the presence of corresponding proteins. The upregulated molecule may be a target nucleic acid or protein that is expressed from the nucleic acid molecules of the composition and methods described herein, e.g., a nucleic acid editor protein produced from recombined transcripts in a REJ split system; an edited target nucleic acid and/or the resulting protein produced therefrom; or a representative marker, surrogate, or functional indicator of a target nucleic acid or protein or an edited target nucleic acid and/or the resulting protein.

Upregulation includes any detectable increase in target nucleic acid/protein. In certain examples, detectable target nucleic acid/protein expression in a cell or cell free system increases by at least 20%, at least 30%, at least 40%, at least 50%, at least 60%, at least 70%, at least 75%, at least 80%, at least 90%, at least 95%, at least 100%, at least 200%, at least 400%, or at least 500% as compared to a control. A control may be an amount of target nucleic acid/protein detected in a corresponding sample not treated with a nucleic acid molecule provided herein. In one example, a control is a relative amount of expression in a normal cell (e.g., a non-recombinant cell that does not include a system provided herein). A control can be compared with: a target nucleic acid or protein that is expressed from the nucleic acid molecules of the composition and methods described herein, e.g., a nucleic acid editor protein produced from recombined transcripts in a REJ split system; an edited target nucleic acid and/or the resulting protein produced therefrom; or a representative marker, surrogate, or functional indicator of a target nucleic acid or protein or an edited target nucleic acid and/or the resulting protein. A control used for comparison may be any appropriate control as determined by one of skill in the art. A positive control may be an amount of a corresponding mRNA and/or protein produced from a full-length construct. A negative control may be an amount of a corresponding mRNA and/or protein produced from an empty or otherwise defective construct.

A control can be compared with a target nucleic acid or protein produced in a cell in the presence of a nucleic acid editor protein expressed from the nucleic acid molecules using the compositions and methods provided herein. As described herein, when a nucleic acid editing protein is expressed in a cell from the nucleic acid molecules using the present compositions and methods, the recombined nucleic acid editing protein may edit its target nucleic acid. The edited nucleic acid and/or a protein produced therefrom may be compared to a positive control, which may be an amount of corresponding nucleic acid, e.g., mRNA, and/or protein produced from the target nucleic acid in a normal cell (e.g., a non-recombinant “normal” cell that does not have the target nucleic acid in need of editing and that does not include a system provided herein).

The edited nucleic acid and/or a protein produced therefrom may be compared to a negative control, which may be an amount of corresponding nucleic acid, e.g., mRNA, and/or protein produced from the target nucleic acid in an untreated cell (e.g., a non-recombinant “mutant” cell having the target nucleic acid in need of editing and that does not include a system provided herein). The detectable target nucleic acid and/or protein expression relative to the detectable positive control target nucleic acid and/or protein expression (e.g., cell or cell free system), respectively, may be 20% to 500% of the expression in a positive control.

The detectable target nucleic acid and/or protein expression in a cell or cell free system relative to expression in a positive control may be about 20% to about 500%. The detectable target nucleic acid and/or protein expression in a cell or cell free system relative to expression in a positive control may be about 20% to about 30%, about 20% to about 40%, about 20% to about 50%, about 20% to about 60%, about 20% to about 70%, about 20% to about 90%, about 20% to about 95%, about 20% to about 100%, about 20% to about 150%, about 20% to about 200%, about 20% to about 500%, about 30% to about 40%, about 30% to about 50%, about 30% to about 60%, about 30% to about 70%, about 30% to about 90%, about 30% to about 95%, about 30% to about 100%, about 30% to about 150%, about 30% to about 200%, about 30% to about 500%, about 40% to about 50%, about 40% to about 60%, about 40% to about 70%, about 40% to about 90%, about 40% to about 95%, about 40% to about 100%, about 40% to about 150%, about 40% to about 200%, about 40% to about 500%, about 50% to about 60%, about 50% to about 70%, about 50% to about 90%, about 50% to about 95%, about 50% to about 100%, about 50% to about 150%, about 50% to about 200%, about 50% to about 500%, about 60% to about 70%, about 60% to about 90%, about 60% to about 95%, about 60% to about 100%, about 60% to about 150%, about 60% to about 200%, about 60% to about 500%, about 70% to about 90%, about 70% to about 95%, about 70% to about 100%, about 70% to about 150%, about 70% to about 200%, about 70% to about 500%, about 90% to about 95%, about 90% to about 100%, about 90% to about 150%, about 90% to about 200%, about 90% to about 500%, about 95% to about 100%, about 95% to about 150%, about 95% to about 200%, about 95% to about 500%, about 100% to about 150%, about 100% to about 200%, about 100% to about 500%, about 150% to about 200%, about 150% to about 500%, or about 200% to about 500%. The detectable target nucleic acid and/or protein expression in a cell or cell free system relative to expression in a positive control may be about 20%, about 30%, about 40%, about 50%, about 60%, about 70%, about 90%, about 95%, about 100%, about 150%, about 200%, or about 500%. The detectable target nucleic acid and/or protein expression in a cell or cell free system relative to expression of a positive control may be at least about 20%, about 30%, about 40%, about 50%, about 60%, about 70%, about 90%, about 95%, about 100%, about 150%, or about 200%. The detectable target nucleic acid and/or protein expression in a cell or cell free system relative to expression in a positive control may be at most about 30%, about 40%, about 50%, about 60%, about 70%, about 90%, about 95%, about 100%, about 150%, about 200%, or about 500%.

The edited nucleic acid and/or a protein produced therefrom may be compared to a negative control, which may be an amount of corresponding nucleic acid, e.g., mRNA, and/or protein produced from the target nucleic acid in an untreated cell (e.g., a non-recombinant “mutant” cell having the target nucleic acid in need of editing and that does not include a system provided herein). The detectable target nucleic acid and/or protein expression relative to the detectable negative control target nucleic acid and/or protein expression, respectively, may be increased by 20% to 500%. The detectable target nucleic acid/protein expression in a cell or cell free system may increase relative to a negative control by about 20% to about 30%, about 20% to about 40%, about 20% to about 50%, about 20% to about 60%, about 20% to about 70%, about 20% to about 90%, about 20% to about 95%, about 20% to about 100%, about 20% to about 150%, about 20% to about 200%, about 20% to about 500%, about 30% to about 40%, about 30% to about 50%, about 30% to about 60%, about 30% to about 70%, about 30% to about 90%, about 30% to about 95%, about 30% to about 100%, about 30% to about 150%, about 30% to about 200%, about 30% to about 500%, about 40% to about 50%, about 40% to about 60%, about 40% to about 70%, about 40% to about 90%, about 40% to about 95%, about 40% to about 100%, about 40% to about 150%, about 40% to about 200%, about 40% to about 500%, about 50% to about 60%, about 50% to about 70%, about 50% to about 90%, about 50% to about 95%, about 50% to about 100%, about 50% to about 150%, about 50% to about 200%, about 50% to about 500%, about 60% to about 70%, about 60% to about 90%, about 60% to about 95%, about 60% to about 100%, about 60% to about 150%, about 60% to about 200%, about 60% to about 500%, about 70% to about 90%, about 70% to about 95%, about 70% to about 100%, about 70% to about 150%, about 70% to about 200%, about 70% to about 500%, about 90% to about 95%, about 90% to about 100%, about 90% to about 150%, about 90% to about 200%, about 90% to about 500%, about 95% to about 100%, about 95% to about 150%, about 95% to about 200%, about 95% to about 500%, about 100% to about 150%, about 100% to about 200%, about 100% to about 500%, about 150% to about 200%, about 150% to about 500%, or about 200% to about 500%. The detectable target nucleic acid/protein expression in a cell or cell free system may increase relative to a negative control by about 20%, about 30%, about 40%, about 50%, about 60%, about 70%, about 90%, about 95%, about 100%, about 150%, about 200%, or about 500%. The detectable target nucleic acid/protein expression in a cell or cell free system may increase relative to a negative control by at least about 20%, about 30%, about 40%, about 50%, about 60%, about 70%, about 90%, about 95%, about 100%, about 150%, or about 200%. The detectable target nucleic acid/protein expression in a cell or cell free system may increase relative to a negative control by at most about 30%, about 40%, about 50%, about 60%, about 70%, about 90%, about 95%, about 100%, about 150%, about 200%, or about 500%.

Under conditions sufficient for: A phrase that is used to describe any environment that permits a desired activity. In one example the desired activity is increased expression or activity of a protein needed to treat a disease. In one example the desired activity is decreased expression or activity of a protein needed to treat a disease. In one example the desired activity is expression of a corrected protein sequence needed to treat a disease. In one example the desired activity is treatment of or slowing the progression of a genetic disease such as DMD (or other genetic disease listed in Tables 1-4) in vivo, for example using the disclosed methods and systems.

Vector: A nucleic acid molecule into which a foreign nucleic acid molecule can be introduced without disrupting the ability of the vector to replicate and/or integrate in a host cell. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides.

A vector can include nucleic acid sequences that permit it to replicate in a host cell, such as an origin of replication. A vector can also include one or more selectable marker genes and other genetic elements. An integrating vector is capable of integrating itself into a host nucleic acid. An expression vector is a vector that contains the necessary regulatory sequences to allow transcription and translation of inserted gene or genes.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector.

In some embodiments, the vector is an AAV, such as AAV serotypes AAV9 or AAVrh.10. In some embodiments, the vector is one that can penetrate the blood-brain barrier, for example following intravenous administration. The adeno-associated virus serotype rh.10 (AAV.rh10) vector partially penetrates the blood-brain barrier, providing high levels and spread of transgene expression.

### II. Overview of Several Embodiments

One approach to curing patients who suffer from genetic diseases is gene editing therapy (generally referred to as gene therapy). In such an approach, the defective gene is replaced by an intact version of it, delivered through e.g., a viral vector, which achieves sustained expression from months to years. Although adeno associated viruses (AAVs) have been used for clinical gene replacement therapy, they have a limited packaging capacity (e.g., about less than 5 kb). Thus, strategies to overcome this packaging limitation are needed to achieve gene replacement of genes that exceed the about 5 kb size limit. For example some promoters alone, coding sequences alone, or the combined promoter+coding sequence, exceed the about 5 kb size limit of an AAV. Thus, such proteins encoded by such promoters and coding sequences can be expressed using the disclosed systems.

Several methods of nucleic acid editing, such as editing of target DNA or RNA molecules, such as gene editing, can be used to upregulate or downregulate expression of a target, as well as correct mutations in a target. Examples of such methods include CRISPR/Cas methods of editing DNA (e.g., using Cas9 or dCas9 DNA endonucleases), CRISPR/Cas methods of editing RNA (e.g., using Cas13d or dCas13d RNA endonucleases), zinc finger nuclease methods of genome editing (e.g., using zinc finger nucleases which include a zinc finger DNA-binding domain and a DNA-cleavage domain), and transcription activator-like effector nucleases (TALENs) based methods of genome editing, (e.g., using transcription activator-like effector nuclease (TALEN) proteins). All of such methods rely on the use of a nucleic acid editing protein, which is a nuclease that can insert, delete, and/or transverse a target nucleic acid sequence (such as a target DNA or RNA sequence) in a cell. Thus, a nucleic acid editing protein can effect the insertion, deletion, and/or substitution of one or more selected nucleotides or ribonucleoties in a target DNA or RNA sequence. However, as discussed above, the cargo limitations of vectors, such as AAV, can make it difficult to produce adequate levels of nucleic acid editing proteins (and in some examples also corresponding gRNAs) to treat disease.

Splicing mediated recombination of two RNA molecules using naturally occurring intron sequences for one or both of the RNA fragments is inefficient. First, these natural intron sequences are sequences from naturally occurring introns and are comprised of a mix of all four RNA nucleotides. Such sequences tend to fold up into structures that can obstruct trans-interaction by forming strong intramolecular base pairs rather than being available for intermolecular interactions. Second, these naturally occurring intron sequences have not evolved to strongly attract the spliceosome components, since exon rather than introns drive the exon definition in higher eukaryotes. These two limitations of previous strategies are addressed herein by designing synthetic intronic sequences that are not found in nature. These synthetic sequences contain elements that strongly attract and stimulate spliceosome recruitment on the one hand while minimizing the secondary structure (and in some examples other structure, such as tertiary structure) that obstructs bringing the two RNA fragments together.

The inventors developed a novel nucleic acid based element that can be used to efficiently reconstitute the coding sequence of large genes from multiple serial fragments. The compositions, systems and method provided herein allow reconstitution of a full-length RNA from the multiple serial fragments. Reconstitution of a full-length RNA, e.g., a messenger RNA transcript, in turn leads to production of the full-length “reconstituted” protein. The disclosed methods and systems differ from prior methods. The disclosed highly efficient synthetic introns utilize an optimal arrangement of RNA elements (or DNA encoding these elements) that efficiently drive the RNA splicing reaction between non-covalently linked RNAs (pre-mRNAs). The method/system is a significant advancement over previous attempts to harness trans-splicing because it generates high levels of functional nucleic acid editing protein that more closely approximate the therapeutic levels of a nucleic acid editing protein to edits a target nucleic acid molecule to treat genetic diseases. The innovation is based on selecting non-natural RNA domains that inherently are incapable of forming strong cis-binding interactions that interfere with trans-interactions with a second RNA having a complementary strand (also having inherently low cis-binding capacity). Intermolecular interactions between binding regions of partnered dimerization domains, e.g., a first dimerization domain and a second dimerization domain, a second dimerization domain and a third dimerization domain, a third dimerization domain and a fourth dimerization domain, a fourth dimerization domain and a fifth dimerization domain, or a fifth dimerization domain and a sixth dimerization domain, may be stronger than intramolecular interactions between a binding region on a single dimerization domain with other sequences in the same dimerization domain. For example, a single stranded kissing loop structure in a kissing loop first dimerization domain may more strongly bind to, hybridize, or associate with its complementary kissing loop structure on a second kissing loop dimerization domain than to other sequences on the same dimerization domain. It is understood that there can be regions within the same dimerization domain that bind more strongly intramolecularly than intermolecularly, for example the stem of a stem-loop structure. The resulting dimerization domains comprise stably available single-stranded binding regions that bind selectively and efficiently to their intended target, i.e., a complementary binding region on a partner dimerization domain. This strategy allows unprecedented reconstitution efficiencies of transcripts synthesized from separate templates, resulting in high levels of target protein or therapeutic nucleic acid production. Many examples of such optimized synthetic nucleic acid molecules and methods for their use are provided herein. These optimized dimerization domains and/or synthetic introns can include non-natural sequences (e.g., sequences not found in human cells and/or not found in another biological system) used in combination with optimized motifs that facilitate RNA splicing (including splice donor, splice acceptor, splice enhancer, and splice branch point sequences). A synthetic nucleic acid can be a non-natural nucleic acid sequence, e.g., a sequence not found in human cells and/or not found in another biological system). By optimizing the trans-dimerization of the RNA strands in the context of the appropriate RNA motifs that mediate efficient splicing, it is demonstrated herein for the first time that two or three different RNAs can be precisely and efficiently covalently linked in the same cell producing high levels of functional nucleic acid editing proteins in vivo and in vitro. Unlike the “hybrid” approach that provides an inefficient combination at the DNA level via DNA recombination that is ultimately followed by RNA splicing in cis to excise the DNA recombination site from the mature transcript, the disclosed method/system promotes a more efficient reaction in which two protein coding RNA fragments are joined together on the pre-mRNA level with less risk of producing recombination products that encode non-functional and/or deleterious products.

The data demonstrate that by using efficient synthetic RNA-dimerization and recombination domains (sRdR domains, also referred to as RNA end-joining (REJ) domains), a nucleic acid editing protein can be efficiently produced by reconstitution of its full-length mRNA from two separate gene fragments expressed from two separate nucleic acid constructs in the same cell. A desired guide RNA, e.g., a gRNA, can be expressed from one of the two constructs, or from a different construct. The disclosed methods and systems can be used to reconstitute transcripts encoding large genes like ABE8e, in order to edit any target nucleic acid to treat any genetic disease. Based on these observations, any genetic disease can be treated, such as ones benefiting from expression of a nucleic acid editing protein (e.g., see disorders listed in Tables 1-4). Other diseases that can be treated using nucleic acid editing proteins include cancer and infectious diseases (such as a bacterial or viral infection). Other applications include research and biotechnology applications.

In some embodiments, the disclosure provides methods for using the nucleic acid editing compositions and systems of the disclosure for treating a subject in need thereof by altering a target nucleic acid sequence in a cell of the subject. Uses for nucleic acid editing are described in the literature, e.g., in U.S. Pat. App. No. 2020/392473, “Novel CRISPR enzymes and systems,” incorporated herein by reference in its entirety.

In some embodiments, the compositions, systems, and methods described herein are used for treating a subject having a disease or disorder by repairing a nucleic acid mutation that causes defective or decreased production of a protein or another gene product, e.g., an RNA. In some embodiments, the defective protein or gene product has reduced activity, is nonfunctional, or is toxic. In some embodiments, the mutation is in a coding or noncoding region of the gene. In some embodiments, the disease or disorder is caused by a mutation in a coding region of a gene that results in a nonfunctional or otherwise defective gene product, and repair of the mutation restores the expression of the functional gene product. In some embodiments, the disease or disorder is caused by a mutation in a regulatory region of a gene, for example, a promoter region or splicing element, and repair of the mutation restores the normal or a desirable level expression of the gene product by upregulation or downregulation. In some embodiments, mutations can be introduced to alter splicing to skip exons, thereby to restoring a normal or desirable level of a functional gene product. In some embodiments, the level of functional gene product is modulated in comparison to a control, e.g., an untreated control.

In some embodiments, a disease gene having a loss-of-function mutation is a disease gene listed in Table 1, reproduced from Table 2 of Chen and Altman, 2017, “Opportunities for developing therapies for rare genetic diseases: focus on gain of function and allostery,” Orphanet Journal of Rare Diseases 12:61, incorporated herein by reference. In some embodiments, the nucleic acid editing compositions, systems, and methods described herein are used to treat a disease listed in Table 1.

TABLE 1
Genetic Diseases Resulting from Loss-of-Function Mutations

| Disease name | [symbol] | Gene/Protein | [symbol] |
|---|---|---|---|
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |

[symbol]

disease names, [symbol] identifiers, mutated genes and known [symbol] are listed. [symbol] are [symbol] from the [symbol] The rest of potential candidates can be found in Additional file [symbol] Table [symbol] [symbol]
indicates data missing or illegible when filed

In some embodiments, the compositions and methods described herein are useful for treating a subject having a disease or disorder by introducing a nucleic acid mutation to disrupt an undesirable genomic sequence and/or downregulate the production of an undesirable gene product in a cell of the subject. In some embodiments, the production of the undesirable genomic sequence and/or undesirable gene product is downregulated by altering a coding region or a noncoding region. In some embodiments, one or more regulatory sequence (e.g., a promoter or enhancer) is altered. In some embodiments, sequences that modulate splicing or other aspects of RNA processing (e.g., poly-adenylation, subcellular localization, or half-life) are altered to disrupt the undesirable genomic sequence and/or downregulate the level of an undesirable gene product. In some embodiments the coding region of the undesirable genomic sequence is altered to introduce a mutation, e.g., a deletion, missense mutation, frameshift mutation, or stop codon, resulting in downregulation of an activity of the undesirable genomic sequence and/or downregulation of production of an undesirable gene product. In some embodiments, the level of the activity and/or gene product is downregulated in comparison to a control, e.g., an untreated control. In some embodiments, an undesirable genomic sequence for targeting using the nucleic acid editing compositions, systems, and methods described herein can include any described in the literature, e.g., an oncogene or a disease gene, having a gain-of function mutation. In some embodiments, the oncogene is any listed in Table 2. In some embodiments, the nucleic acid editing compositions, systems, and methods described herein are used to treat a cancer listed in Table 2.

TABLE 2
Oncogenes

| Oncogene | Function/Activation | Cancer* |
|---|---|---|
| ABL1 | Promotes cell growth through tyrosine | Chronic myelogenous leukemia |
| | kinase activity | |
| AFF4/MLLT11 | Fusion affects the MLLT11 | Acute leukemias |
| | transcription factor/methyltransferase. | |
| | MLLT11 is also called HRX, ALL1 | |
| | and HTRX1 | |
| AKT2 | Encodes a protein-serine/threonine | Ovarian cancer |
| | kinase | |
| ALK | Encodes a receptor tyrosine kinase | Lymphomas |
| ALK/NPM | Translocation creates fusion protein | Large cell lymphomas |
| | with nucleophosmin(npm) | |
| RUNX1 (AML1) | Encodes a transcription factor | Acute myeloid leukemia |
| RUNX1/MTG8(ETO) | New fusion protein created by | Acute leukemias |
| | translocation | |
| AXL | Encodes a receptor tyrosine kinase | Hematopoietic cancers |
| BCL-2, 3, 6 | Block apoptosis (programmed cell | B-cell lymphomas and leukemias |
| | death) | |
| BCR/ABL | New protein created by fusion of bcr | Chronic myelogenous and acute |
| | and abl triggers unregulated cell | lymphotic leukemia |
| | growth | |
| MYC (c-MYC) | Transcription factor that promotes cell | Leukemia; breast, stomach, lung, |
| | proliferation and DNA synthesis | cervical, and colon carcinomas; |
| | | neuroblastomas and glioblastomas |
| MCF2 (DBL) | Guanine nucleotide exchange factor | Diffuse B-cell lymphoma |
| DEK/NUP214 | New protein created by fusion | Acute myeloid leukemia |
| TCF3/PBX1 | New protein created by fusion | Acute pre B-cell leukemia; TCF3 also |
| | | called E2A |
| EGFR | Cell surface receptor that triggers cell | Squamous cell carcinoma, |
| | growth through tyrosine kinase activity | glioblastomas, lung cancer |
| MLLT11 | Fusion protein created by a | Acute leukemias |
| | translocation t(11; 19). | |
| ERG/FUS | Fusion protein created by t(16:21) | Myeloid leukemia |
| | translocation. The erg protein is a | |
| | transcription factor. | |
| ERBB2 | Cell surface receptor that triggers cell | Breast, salivary gland, and ovarian |
| | growth through tyrosine kinase | carcinomas |
| | activity; also known as HER2 or neu | |
| ETS1 | Transcription factor | Lymphoma |
| EWSR1/FLI1 | Fusion protein created by t(11:22) | Ewing Sarcoma |
| | translocation. | |
| CSF1R | Tyrosine kinase | Sarcoma |
| FOS | Transcription factor for API | Osteosarcoma |
| FES | Tyrosine kinase | Sarcoma |
| GLI1 | Transcription factor | Glioblastoma |
| GNAS (GSP) | Membrane associated G protein | Thyroid carcinoma |
| HER2/neu | Overexpression of signaling kinase due | Breast and cervical carcinomas |
| | to gene amplification | |
| TLX1 | Transcription factor; aka HOX11 | Acute T-cell leukemia |
| FGF4 | Encodes fibroblast growth factor; aka | Breast and squamous cell carcinomas |
| | HST1 | |
| IL3 | Cell signaling molecule | Acute pre B-cell leukemia |
| FGF3 (INT-2) | Encodes a fibroblast growth factor | Breast and squamous cell carcinomas |
| JUN | Transcription factor for API | Sarcoma |
| KIT | Tyrosine kinase | Sarcoma |
| FGF4 (KS3) | Herpes virus encoded growth factor | Kaposi's sarcoma |
| K-SAM | Fibroblast growth factor receptor | Stomach carcinomas |
| AKAP13 | Guanine nucleotide exchange factor; | Myeloid leukemias |
| | aka LBC | |
| LCK | Tyrosine kinase | T-cell lymphoma |
| LMO1, LMO2 | Transcription factors | T-cell lymphoma |
| MYCL | Transcription factor | Lung carcinomas |
| LYL1 | Transcription factor | Acute T-cell leukemia |
| NFKB2 | Transcription factor. Also called LYT- | B-cell lymphoma |
| | 10 | |
| NFKB2/Cα1 | Fusion protein formed by | |
| | the (10; 14)(q24; q32) translocation of | |
| | NFKB2 next to the C alpha 1 | |
| | immunoglobulin locus. | |
| MAS1 | Angiotensin receptor | Mammary carcinoma |
| MDM2 | Encodes a protein that inhibits and | Sarcomas |
| | leads to the degradation of p53 | |
| MLLT11 | Transcription factor/methyltransferase | Acute myeloid leukemia |
| | (also called HRX and ALL1) | |
| MOS | Serine/threonine kinase | Lung cancer |
| RUNX1T1 | Fusion of transcription repressor to | Acute leukemias |
| | factor to a transcription factor. Also | |
| | known as MTG8 and AML1-MTG8 | |
| MYB | Transcription factor | Colon carcinoma and leukemias |
| MYH11/CBFB | New protein created by fusion of | Acute myeloid leukemia |
| | transcription factors via an inversion in | |
| | chromosome 16. | |
| NEU | Tyrosine kinase. Also called ERBB2 | Glioblastomas, and squamous cell |
| | or HER2 | carcinomas |
| MYCN | Cell proliferation and DNA synthesis | Neuroblastomas, retinoblastomas, and |
| | | lung carcinomas |
| MCF2L (OST) | Guanine nucleotide exchange factor | Osteosarcomas |
| PAX-5 | Transcription factor | Lympho-plasmacytoid B-cell lymphoma |
| PBX1/E2A | Fusion protein formed via t(1:19) | Acute pre B-cell leukemia |
| | translocation. Transcription factor | |
| PIM1 | Serine/threonine kinase | T-cell lymphoma |
| CCND1 | Encodes cyclin D1. Involved in cell | Breast and squamous cell carcinomas |
| | cycle regulation. Also called PRAD1 | |
| RAF1 | Serine/threonine kinase | Many cancer types |
| RARA/PML | Fusion protein caused by t(15:17) | Acute premyelocytic leukemia |
| | translocation. Retinoic acid receptor. | |
| HRAS | G-protein. Signal transduction. | Bladder carcinoma |
| KRAS | G-protein. Signal transduction | Lung, ovarian, and bladder carcinoma |
| NRAS | G-protein. Signal transduction | Breast carcinoma |
| REL/NRG | Fusion protein formed by deletion in | B-cell lymphoma |
| | chromosome 2. Transcription factor. | |
| RET | Cell surface receptor. Tyrosine kinase | Thyroid carcinomas, multiple endocrine |
| | | neoplasia type 2 |
| RHOM1, RHOM2 | Transcription factors aka LMO1 and | Acute T-cell leukemia |
| | LMO2 | |
| ROS1 | Tyrosine kinase | Sarcoma |
| SKI | Transcription factor | Carcinomas |
| SIS (aka PDGFB) | Growth factor | Glioma, fibrosarcoma |
| SET/CAN | Fusion protein formed by | Acute myeloid leukemia |
| | rearrangement of chromosome 9. | |
| | Protein localization | |
| SRC | Tyrosine kinase | Sarcomas |
| TAL1, TAL2 | Transcription factor. TAL1 is also | Acute T-cell leukemia |
| | called SCL | |
| NOTCH1 (TAN1) | Altered form of Notch (a cellular | Acute T-cell leukemia |
| | receptor) formed by t(7:9) translocation | |
| TIAM1 | Guanine nucleotide exchange factor | T-lymphoma |
| TSC2 | GTPase activator | Renal and brain tumors |
| NTRK1 | Receptor tyrosine kinase | Colon and thyroid carcinomas |

In some embodiments, a disease gene having a gain-of-function mutation is a neurodegenerative disease gene. In some embodiments, the neurodegenerative disease gene is any listed in Table 3, reproduced from Table 1 of Chen and Altman, 2017. In some embodiments, the nucleic acid editing compositions, systems, and methods described herein are used to treat a neurodegenerative disease listed in Table 3.

TABLE 3
Genetic Diseases Resulting from Gain-of-Function Mutations

| Type | Diseases | [symbol] | Gene/Protein |
|---|---|---|---|
| Overactivation | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| Gene duplication | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| [symbol] | [symbol] | [symbol] | [symbol] |
| | [symbol] | [symbol] | [symbol] |
| | [symbol] | [symbol] | [symbol] |
| | [symbol] | [symbol] | [symbol] |

Corresponding disease names, [symbol] identifiers, and mutated genes are listed. Diseases are grouped by the molecular mechanism as indicated in the type column. The rest of potential candidates can be found in Additional file [symbol] Table [symbol] [symbol]
indicates data missing or illegible when filed

To address some of the limitations with existing strategies for reconstitution of fragmented genes from multiple AAVs, provided herein is a system that serially aligns and recombines two or more individual synthetic RNA molecules in the target cell. Each individual synthetic RNA molecule includes a synthetic intron sequence, containing a dimerization domain and elements needed for RNA splicing, which upon binding of dimerization domains to one another in the correct order, mediates efficient RNA recombination of individual fragments. In one example, reconstitution of a coding sequence from two fragments is achieved by appending a first synthetic intron (A) to the 3′ end of the N-terminal coding fragment and a complimentary second synthetic domain (A′) to the 5′ end of the C-terminal coding fragment. The two RNAs are recombined by a cell's intrinsic RNA splicing machinery (i.e., the spliceosome machinery). The synthetic intron domains contain two functional elements: (1) a dimerization domain to mediate base pairing between the two halves that are to be recombined and (2) a domain optimized to efficiently recruit the splicing machinery to mediate efficient reconstitution of the two RNA molecules. The synthetic intron domain can include elements to prevent unspliced RNA from encoding protein. In some examples, a synthetic intron includes a sequence having at least 50% at least 60%, at least 70%, at least 75%, 80%, at least 85%, at least 90%, at least 95%, at least 98%, at least 99%, or 100% sequence identity to any synthetic intron provided in SEQ ID NOS: 159, 160, 161, 162, 163, 164, 165 and 166 (e.g., see FIGS. 10A-10Z). In some examples, a synthetic intron is an RNA molecule encoded by a sequence having at least 50% at least 60%, at least 70%, at least 75%, 80%, at least 85%, at least 90%, at least 95%, at least 98%, at least 99%, or 100% sequence identity to any synthetic intron provided in SEQ ID NOS: 159, 160, 161, 162, 163, 164, 165, and 166, but without the provided promoter sequence). One skilled in the art will appreciate that any of the molecules provided in SEQ ID NOS: 159, 160, 161, 162, 163, 164, 165, and 166 can be modified to replace the protein coding portions (e.g., 114 and 164 of FIG. 6A) with another nucleic acid editing protein coding sequence of interest (e.g., YFP coding sequence of SEQ ID NO: 1, 2, 22 or 23 can be replaced with a Cas9 or Cas13d protein coding sequence). Thus, also provided herein are synthetic intron molecules having at least 80%, at least 85%, at least 90%, at least 95%, at least 98%, at least 99%, or 100% sequence identity to any synthetic intron portion provided in SEQ ID NO: 159, 160, 161, 162, 163, 164, 165, and 166. Also provided are synthetic intron RNA molecules encoded by a sequence having at least 50% at least 60%, at least 70%, at least 75%, 80%, at least 85%, at least 90%, at least 95%, at least 98%, at least 99%, or 100% sequence identity to any synthetic intron provided in SEQ ID NOS: 159, 160, 161, 162, 163, 164, 165, and 166, but without the provided promoter sequence).

Exemplary dimerization domains were bioinformatically selected to minimize/optimize their internal secondary/tertiary structure. The dimerization domains tested contained long stretches of low diversity nucleotide sequences to avoid intramolecular annealing. By avoiding intramolecular annealing, these dimerization domains are present in an open configuration and therefore are available for pairing with the corresponding complementary dimerization domain sequence. The synthetic intron domains contain intronic splice enhancing elements which lead to efficient recruitment of the splicing machinery.

The disclosed RNA molecules are designed to have at least an open and available single-stranded region that is available to bind to the complementary dimerization domain to allow efficient splicing and recombination of the RNAs. In some examples, this is achieved by utilizing only purines or only pyrimidines for the binding domains. Due to the inability of purines to pair with themselves (and pyrimidines likewise) these stretches of RNA have an open predicted structure.

RNA molecules are present as a single strand in the cells. Being single stranded they are inherently prone to hybridize to themselves and thereby form strong secondary and tertiary structures. The most stable base pairs will be G with C, A with U, and the G with U wobble pair. Thermodynamically, the pairing of two bases is favored over an open configuration. To design efficient synthetic nucleic acid molecules, two dimerization domains having complementarity to one another are present in an open configuration such that the dimerization domains are available for inter-molecular base pairing. To avoid intra-molecular base pairing in between other parts of the synthetic nucleic acid molecules, a long stretch of non-diverse sequences containing incompatible bases can be included. For example, a long stretch of pyrimidines (i.e., C and T) or purines (i.e., A and G) can be present in the synthetic nucleic acid molecules. Pyrimidines cannot form canonical base pairs with other pyrimidines, purines cannot form canonical base pairs with other purines. Such a stretch of purines or pyrimidines can range from a couple bases to a couple hundreds of bases. Since these stretches cannot intra-molecularly bind, they are available for inter-molecular base pairing with a complementary fragment. For example, the synthetic nucleic acid molecules A and A′ may be configured with A containing a pyrimidine stretch (e.g., 5′-CCUU ( . . . ) CCUU-3′) and A′ containing the complementary purine sequence (e.g., 5′-AAGG ( . . . ) AAGG-3′).

The disclosed synthetic nucleic acid molecules (e.g., RNA or DNA encoding the RNA) are designed to minimize any off-target binding to incorrect sites in the genome. Off target binding can be reduced by altering the sequence of the nucleic acid molecule.

The same design principle, that is the use of hypodiverse stretches of RNA bases to achieve open synthetic nucleic acid configurations, can be extended to using stretches of single bases e.g. using a series of Gs that would base pair with a series of Cs and a series of As that would base pair with a series of Us, in the dimerization domains.

To increase recombination of two or more synthetic nucleic acid molecules, the following methods can be used. RNA splicing depends on the recruitment of spliceosome components to the 5′ end of the intron (the splice donor site) and the 3′ end of the intron (the splice acceptor site, with its associated branch point sequence and the polypyrimidine tract). Different ribonucleoproteins are recruited to the intron through base pairing of protein associated small nuclear RNA (snRNA) with intronic sequences. By placing perfect match consensus sequences into the RNA dimerization and recombination domains, the recruitment of spliceosome components can be facilitated which in turn enhances the efficiency of spliceosome mediated recombination. Previously characterized intronic splice enhancer sequences can recruit additional splicing promoting factors that are referred to as intronic splice enhancers.

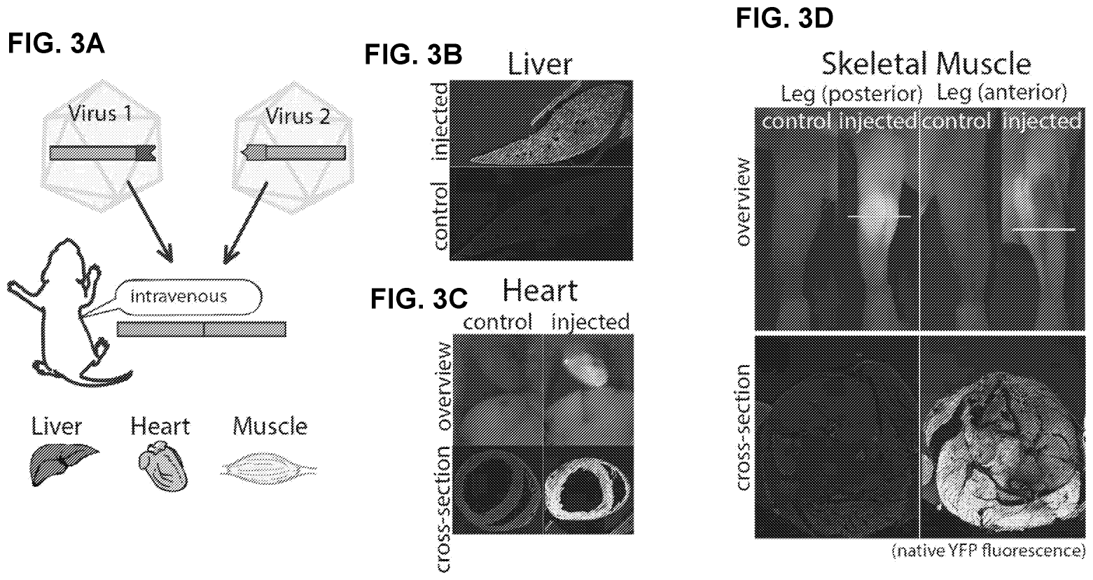

In some examples, instead of using naturally occurring RNA sequences for the RNA splicing sequences, consensus sequences are used. For example, consensus sequences can be used for any of the sequences that are involved in splicing, including splice donor, splice acceptor, splice enhancer and splice branch point sequences. With these synthetic nucleic acid molecules, two (or more) RNA molecules can be serially joined together in a cell ex vivo, in vitro, or in vivo. Outside of the encoded synthetic intronic domains, synthetic nucleic acid molecules can include any promoter and coding sequence. For example, two synthetic nucleic acid molecules could carry two halves of a single nucleic acid editing gene. This was tested in vitro and in vivo by reconstituting two halves of a yellow fluorescent protein (YFP), and was shown to be efficient (see FIGS. 3A-3D).

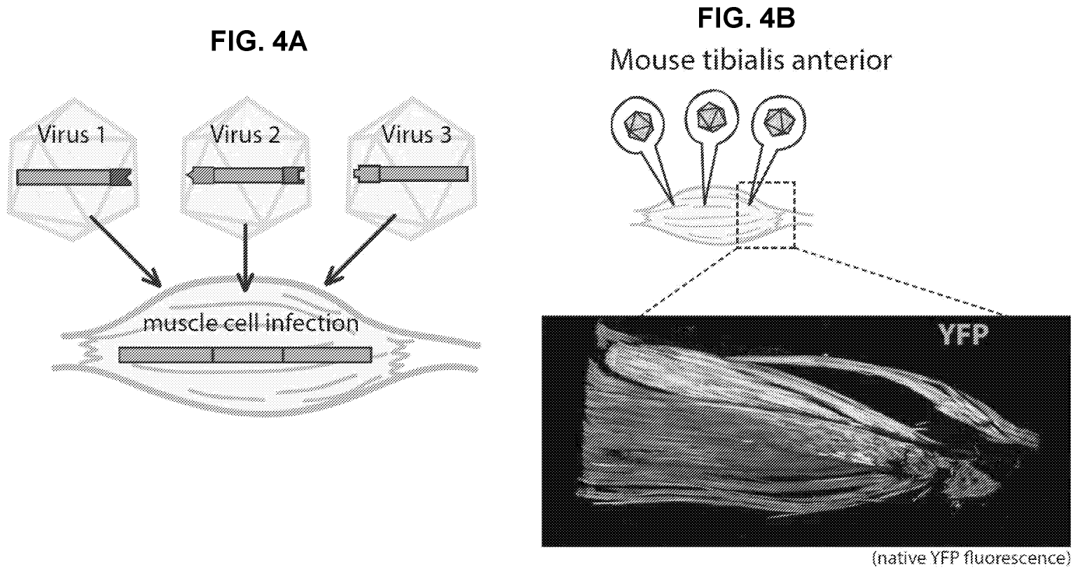

The modular nature of the synthetic nucleic acid molecules allowed for testing the efficiency of achieving serial recombination (i.e., >2) of multiple RNA fragments using a combinatorial set of optimized complimentary dimerization domains (FIGS. 4A-4B). A three-way split yellow fluorescent protein was efficiently reconstituted and expressed at high levels in >80% of transfected cells.

These results demonstrate that a single RNA molecule can be reconstituted from at least three different synthetic nucleic acid molecules, such as when expression of a nucleic acid editing protein that has a promoter and/or a coding sequence that is too long to fit into a single gene therapy vector such as AAV.

In some examples, the synthetic nucleic acid molecules, e.g., synthetic DNA molecules, of the inventive compositions, systems, kits, and methods, are produced by transcription of an RNA virus genome by reverse transcriptase.

The disclosed system allows for the efficient RNA recombination between individual fragments. In some examples, reconstitution (i.e., splicing or recombination) efficiency achieved using the compositions, systems or methods of the disclosure is determined using any suitable method known to one of skill in the art. In some examples, reconstitution efficiency is represented by a measure of correctly joined RNA relative to a control RNA, or a measure of full-length nucleic acid editing protein or protein activity relative to that of a control protein. In some examples the control RNA is the unjoined RNA, wherein reconstitution efficiency is represented by a measure of joined RNA relative to unjoined RNA. This measurement can be made by detecting and comparing junction RNA and the unjoined 3′ RNA species 3′ (e.g., junction RNA: 3′ RNA). In some examples wherein more than two RNAs are joined, joining at either or all junctions are evaluated. In some examples, reconstitution efficiency is represented by a measure of full-length or active nucleic acid editing protein relative to a protein fragment or inactive protein.

In some examples, the reconstitution, recombination or splicing efficiency (a measure of the correct joining of the two or more different coding sequences present on different RNA molecules, and/or the production of the desired full-length protein) is about 10% to about 100%. The synthetic intron domain can include elements to prevent unspliced RNA from encoding protein. In some examples, the reconstitution efficiency is about 10% to about 15%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 10% to about 100%, about 15% to about 20%, about 15% to about 25%, about 15% to about 30%, about 15% to about 40%, about 15% to about 50%, about 15% to about 60%, about 15% to about 70%, about 15% to about 80%, about 15% to about 90%, about 15% to about 100%, about 20% to about 25%, about 20% to about 30%, about 20% to about 40%, about 20% to about 50%, about 20% to about 60%, about 20% to about 70%, about 20% to about 80%, about 20% to about 90%, about 20% to about 100%, about 25% to about 30%, about 25% to about 40%, about 25% to about 50%, about 25% to about 60%, about 25% to about 70%, about 25% to about 80%, about 25% to about 90%, about 25% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some examples, the reconstitution efficiency is about 10%, about 15%, about 20%, about 25%, about 30%, about 40%, about 50%, about 60%, about 70%, about 80%, about 90%, or about 100%. In some examples, the reconstitution efficiency is at least about 10%, about 15%, about 20%, about 25%, about 30%, about 40%, about 50%, about 60%, about 70%, about 80%, or about 90%. In some examples, the reconstitution efficiency is at most about 15%, about 20%, about 25%, about 30%, about 40%, about 50%, about 60%, about 70%, about 80%, about 90%, or about 100%.

In some examples, the reconstitution, recombination or splicing efficiency (in this example a measure of the correct joining of two different nucleic acid editing protein coding sequences present on different RNA molecules, and/or the production of the desired nucleic acid editing full-length protein, wherein the two different nucleic acid editing protein coding sequences encode a transcript of about 3200 nt to 9000 nt, such as about 4000 to 9000 nt, about 4400 to 9000 nt, about 3200 to 4000 nt, about 3200 to 3600 nt, for example about 4500 nt, about 4000 nt, about 3800 nt, about 3600 nt, or about 3200 nt), is about 10% to about 100%. In some examples, the reconstitution efficiency using a two-part system is about 10% to about 15%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 10% to about 100%, about 15% to about 20%, about 15% to about 25%, about 15% to about 30%, about 15% to about 40%, about 15% to about 50%, about 15% to about 60%, about 15% to about 70%, about 15% to about 80%, about 15% to about 90%, about 15% to about 100%, about 20% to about 25%, about 20% to about 30%, about 20% to about 40%, about 20% to about 50%, about 20% to about 60%, about 20% to about 70%, about 20% to about 80%, about 20% to about 90%, about 20% to about 100%, about 25% to about 30%, about 25% to about 40%, about 25% to about 50%, about 25% to about 60%, about 25% to about 70%, about 25% to about 80%, about 25% to about 90%, about 25% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some examples, the reconstitution efficiency is about 10%, about 15%, about 20%, about 25%, about 30%, about 40%, about 50%, about 60%, about 70%, about 80%, about 90%, or about 100%. In some examples, the reconstitution efficiency is at least about 10%, about 15%, about 20%, about 25%, about 30%, about 40%, about 50%, about 60%, about 70%, about 80%, or about 90%. In some examples, the reconstitution efficiency is at most about 15%, about 20%, about 25%, about 30%, about 40%, about 50%, about 60%, about 70%, about 80%, about 90%, or about 100%.

In some examples, the reconstitution, recombination or splicing efficiency (in this example a measure of the correct joining of the two different nucleic acid editing protein coding sequences present on different RNA molecules, and/or the production of the desired full-length nucleic acid editing protein, wherein the two different nucleic acid editing protein coding sequences encode a transcript of about 4000 nt), is about 40% to about 60%, such as about 40% to about 50%, about 42% to about 47%, for example about 45%.

In some examples, the reconstitution, recombination or splicing efficiency (in this example a measure of the correct joining of the two different nucleic acid editing protein coding sequences present on different RNA molecules, and/or the production of the desired nucleic acid editing full-length protein, wherein the two different nucleic acid editing protein coding sequences encode a transcript of about 3800 nt), is about 40% to about 60%, such as about 40% to about 50%, about 42% to about 47%, for example about 45%.

In some examples, the reconstitution, recombination or splicing efficiency (in this example a measure of the correct joining of the two different nucleic acid editing protein coding sequences present on different RNA molecules, and/or the production of the desired nucleic acid editing full-length protein, wherein the two different nucleic acid editing protein coding sequences encode a transcript of about 3600 nt), is about 25% to about 50%, such as about 30% to about 40%, for example about 35%.

In some examples, the reconstitution, recombination or splicing efficiency (in this example a measure of the correct joining of the two different nucleic acid editing protein coding sequences present on different RNA molecules, and/or the production of the desired nucleic acid editing full-length protein, wherein the two different nucleic acid editing protein coding sequences encode a transcript of about 3200 nt), is about 25% to about 50%, such as about 30% to about 40%, for example about 35%.

In some examples, the reconstitution, recombination or splicing efficiency (in this example a measure of the correct joining of three different nucleic acid editing protein coding sequences present on different RNA molecules, and/or the production of the desired full-length nucleic acid editing protein, wherein the three different nucleic acid editing protein coding sequences encode a transcript of about 3200 nt to about 13,500 nt, such as about 4000 nt to about 5,000 nt, about 4000 nt to about 13,500 nt, about 6000 nt to about 12,000 nt, about 6000 nt to about 10,000 nt, or about 8000 nt to about 12,000 nt, for example up to about 13,500 nt), is about 10% to about 100%. In some examples, the reconstitution efficiency using a three-part system is about 10% to about 15%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 10% to about 100%, about 15% to about 20%, about 15% to about 25%, about 15% to about 30%, about 15% to about 40%, about 15% to about 50%, about 15% to about 60%, about 15% to about 70%, about 15% to about 80%, about 15% to about 90%, about 15% to about 100%, about 20% to about 25%, about 20% to about 30%, about 20% to about 40%, about 20% to about 50%, about 20% to about 60%, about 20% to about 70%, about 20% to about 80%, about 20% to about 90%, about 20% to about 100%, about 25% to about 30%, about 25% to about 40%, about 25% to about 50%, about 25% to about 60%, about 25% to about 70%, about 25% to about 80%, about 25% to about 90%, about 25% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some examples, the reconstitution efficiency is about 10%, about 15%, about 20%, about 25%, about 30%, about 40%, about 50%, about 60%, about 70%, about 80%, about 90%, or about 100%. In some examples, the reconstitution efficiency is at least about 10%, about 15%, about 20%, about 25%, about 30%, about 40%, about 50%, about 60%, about 70%, about 80%, or about 90%. In some examples, the reconstitution efficiency is at most about 15%, about 20%, about 25%, about 30%, about 40%, about 50%, about 60%, about 70%, about 80%, about 90%, or about 100%.

In some examples, the reconstitution, recombination or splicing efficiency (in this example a measure of the correct joining of four different nucleic acid editing protein coding sequences present on different RNA molecules, and/or the production of the desired full-length nucleic acid editing protein, wherein the four different nucleic acid editing protein coding sequences encode a transcript of about 3200 nt to about 18,000 nt, such as about 4000 nt to about 18,000 nt, about 4000 nt to about 5,000 nt, about 10,000 nt to about 18,000 nt, about 15,000 nt to about 18,000 nt, or about 12,000 nt to about 15,000 nt, for example up to about 18,000 nt), is about 10% to about 100%. In some examples, the reconstitution efficiency using a four-part system is about 10% to about 15%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 10% to about 100%, about 15% to about 20%, about 15% to about 25%, about 15% to about 30%, about 15% to about 40%, about 15% to about 50%, about 15% to about 60%, about 15% to about 70%, about 15% to about 80%, about 15% to about 90%, about 15% to about 100%, about 20% to about 25%, about 20% to about 30%, about 20% to about 40%, about 20% to about 50%, about 20% to about 60%, about 20% to about 70%, about 20% to about 80%, about 20% to about 90%, about 20% to about 100%, about 25% to about 30%, about 25% to about 40%, about 25% to about 50%, about 25% to about 60%, about 25% to about 70%, about 25% to about 80%, about 25% to about 90%, about 25% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some examples, the reconstitution efficiency is about 10%, about 15%, about 20%, about 25%, about 30%, about 40%, about 50%, about 60%, about 70%, about 80%, about 90%, or about 100%. In some examples, the reconstitution efficiency is at least about 10%, about 15%, about 20%, about 25%, about 30%, about 40%, about 50%, about 60%, about 70%, about 80%, or about 90%. In some examples, the reconstitution efficiency is at most about 15%, about 20%, about 25%, about 30%, about 40%, about 50%, about 60%, about 70%, about 80%, about 90%, or about 100%. In some examples, the compositions, systems or methods of the disclosure are evaluated by determining an RNA or protein production level using any suitable method known to one of skill in the art.

In some examples, the RNA production level is represented by a measure of correctly joined RNA relative to a control RNA, or a measure of full-length protein relative to a control. In some examples the control RNA is a corresponding mutant RNA or an endogenous RNA. For example, the ratio of the amount of joined RNA to the amount of mutant or endogenous RNA produced in the transfected cell is compared with same ratio in nontransfected cells, to determine the production level of the correctly joined RNA. In some examples, the ratio of the amount of the correctly joined RNA, full-length protein, or the protein activity, to the amount of the control RNA, or the amount or activity of the control protein, are compared.

In some examples, the RNA production level achieved is 5% to 100%. In some examples, the RNA production level achieved is about 5% to about 100%. In some examples, the RNA production level achieved is about 5% to about 10%, about 5% to about 20%, about 5% to about 25%, about 5% to about 30%, about 5% to about 40%, about 5% to about 50%, about 5% to about 60%, about 5% to about 70%, about 5% to about 80%, about 5% to about 90%, about 5% to about 100%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 10% to about 100%, about 20% to about 25%, about 20% to about 30%, about 20% to about 40%, about 20% to about 50%, about 20% to about 60%, about 20% to about 70%, about 20% to about 80%, about 20% to about 90%, about 20% to about 100%, about 25% to about 30%, about 25% to about 40%, about 25% to about 50%, about 25% to about 60%, about 25% to about 70%, about 25% to about 80%, about 25% to about 90%, about 25% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some examples, the RNA production level achieved is about 5%, about 10%, about 20%, about 25%, about 30%, about 40%, about 50%, about 60%, about 70%, about 80%, about 90%, or about 100%. In some examples, the RNA production level achieved is at least about 5%, about 10%, about 20%, about 25%, about 30%, about 40%, about 50%, about 60%, about 70%, about 80%, or about 90%. In some examples, the RNA production level achieved is at most about 10%, about 20%, about 25%, about 30%, about 40%, about 50%, about 60%, about 70%, about 80%, about 90%, or about 100%.

In some examples, the protein production level is represented by a measure of the amount of full-length nucleic acid editing protein or nucleic acid editing protein activity relative to that of a control protein.

In some examples the control protein is a corresponding mutant protein or an endogenous protein. For example, the ratio of the amount of full-length nucleic acid editing protein or protein activity to the amount of mutant or endogenous protein produced in the transfected cell is compared with same ratio in nontransfected cells. In some examples, the control protein is the full-length nucleic acid editing protein produced in, e.g., a cell that is engineered to express a control full-length protein (wherein the cell is not transfected with the inventive constructs) or a non-transfected cell from a normal subject that expresses a control full-length protein, and the protein production level is determined by measuring the amount or activity of the nucleic acid editing protein in the transfected cell and comparing it to that of the control protein. In some examples, the control protein is a mutant form of the protein, produced in a cell that is transfected or nontransfected with the construct, and the amount of full-length protein or protein activity is compared with that of the control protein to determine the protein production level. In some examples, the amount of full-length protein or protein activity is compared with that of an endogenous, or housekeeping, protein to determine the protein production level.

In some examples, the protein production level achieved is about 1% to about 100%. In some examples, the protein production level achieved is about 10% to about 100%. In some examples, the protein production level achieved is about 10% to about 20%, about 10% to about 30%, about 10% to about 40%, about 10% to about 50%, about 10% to about 60%, about 10% to about 70%, about 10% to about 75%, about 10% to about 80%, about 10% to about 85%, about 10% to about 90%, about 10% to about 100%, about 20% to about 30%, about 20% to about 40%, about 20% to about 50%, about 20% to about 60%, about 20% to about 70%, about 20% to about 75%, about 20% to about 80%, about 20% to about 85%, about 20% to about 90%, about 20% to about 100%, about 30% to about 40%, about 30% to about 50%, about 30% to about 60%, about 30% to about 70%, about 30% to about 75%, about 30% to about 80%, about 30% to about 85%, about 30% to about 90%, about 30% to about 100%, about 40% to about 50%, about 40% to about 60%, about 40% to about 70%, about 40% to about 75%, about 40% to about 80%, about 40% to about 85%, about 40% to about 90%, about 40% to about 100%, about 50% to about 60%, about 50% to about 70%, about 50% to about 75%, about 50% to about 80%, about 50% to about 85%, about 50% to about 90%, about 50% to about 100%, about 60% to about 70%, about 60% to about 75%, about 60% to about 80%, about 60% to about 85%, about 60% to about 90%, about 60% to about 100%, about 70% to about 75%, about 70% to about 80%, about 70% to about 85%, about 70% to about 90%, about 70% to about 100%, about 75% to about 80%, about 75% to about 85%, about 75% to about 90%, about 75% to about 100%, about 80% to about 85%, about 80% to about 90%, about 80% to about 100%, about 85% to about 90%, about 85% to about 100%, or about 90% to about 100%. In some examples, the protein production level achieved is about 10%, about 20%, about 30%, about 40%, about 50%, about 60%, about 70%, about 75%, about 80%, about 85%, about 90%, or about 100%. In some examples, the protein production level achieved is at least about 10%, about 20%, about 30%, about 40%, about 50%, about 60%, about 70%, about 75%, about 80%, about 85%, or about 90%. In some examples, the protein production level achieved is at most about 20%, about 30%, about 40%, about 50%, about 60%, about 70%, about 75%, about 80%, about 85%, about 90%, or about 100%.

In some examples, the protein activity level achieved is about 50% to about 100%. In some examples, the protein activity level achieved is about 50% to about 100%. In some examples, the protein activity level achieved is about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to about 100%, about 55% to about 60%, about 55% to about 65%, about 55% to about 70%, about 55% to about 75%, about 55% to about 80%, about 55% to about 85%, about 55% to about 90%, about 55% to about 95%, about 55% to about 100%, about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some examples, the protein activity level achieved is about 50%, about 55%, about 60%, about 65%, about 70%, about 75%, about 80%, about 85%, about 90%, about 95%, or about 100%. In some examples, the protein activity level achieved is at least about 50%, about 55%, about 60%, about 65%, about 70%, about 75%, about 80%, about 85%, about 90%, or about 95%. In some examples, the protein activity level achieved is at most about 55%, about 60%, about 65%, about 70%, about 75%, about 80%, about 85%, about 90%, about 95%, or about 100%.

In some examples, the amount of correctly joined RNA or full-length nucleic acid editing protein produced in a cell, for example in combination with expression of one or more gRNAs, is sufficient to ameliorate or cure a condition or disease in a subject, as understood by one of skill in the art for the particular condition or disease. In some examples, the amount of correctly joined RNA or full-length nucleic acid editing protein (for example in combination with expression of one or more gRNAs) produced in a cell is an effective amount. In some examples, this amount is equivalent to about 50% to 100% the amount of the RNA or protein produced in a normal cell. In some examples, this amount is equivalent to about 40% to about 100% the amount of the RNA or protein produced in a normal cell. In some examples, this amount is equivalent to about 40% to about 45%, about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 100%, about 45% to about 50%, about 45% to about 55%, about 45% to about 60%, about 45% to about 65%, about 45% to about 70%, about 45% to about 75%, about 45% to about 80%, about 45% to about 85%, about 45% to about 90%, about 45% to about 100%, about 50% to about 55%, about 50% to about 60%, about 50% to about 65%, about 50% to about 70%, about 50% to about 75%, about 50% to about 80%, about 50% to about 85%, about 50% to about 90%, about 50% to about 100%, about 55% to about 60%, about 55% to about 65%, about 55% to about 70%, about 55% to about 75%, about 55% to about 80%, about 55% to about 85%, about 55% to about 90%, about 55% to about 100%, about 60% to about 65%, about 60% to about 70%, about 60% to about 75%, about 60% to about 80%, about 60% to about 85%, about 60% to about 90%, about 60% to about 100%, about 65% to about 70%, about 65% to about 75%, about 65% to about 80%, about 65% to about 85%, about 65% to about 90%, about 65% to about 100%, about 70% to about 75%, about 70% to about 80%, about 70% to about 85%, about 70% to about 90%, about 70% to about 100%, about 75% to about 80%, about 75% to about 85%, about 75% to about 90%, about 75% to about 100%, about 80% to about 85%, about 80% to about 90%, about 80% to about 100%, about 85% to about 90%, about 85% to about 100%, or about 90% to about 100% the amount of the RNA or protein produced in a normal cell. In some examples, this amount is equivalent to about 40%, about 45%, about 50%, about 55%, about 60%, about 65%, about 70%, about 75%, about 80%, about 85%, about 90%, or about 100% the amount of the RNA or protein produced in a normal cell. In some examples, this amount is equivalent to about at least about 40%, about 45%, about 50%, about 55%, about 60%, about 65%, about 70%, about 75%, about 80%, about 85%, or about 90% the amount of the RNA or protein produced in a normal cell. In some examples, this amount is equivalent to about at most about 45%, about 50%, about 55%, about 60%, about 65%, about 70%, about 75%, about 80%, about 85%, about 90%, or about 100% the amount of the RNA or protein produced in a normal cell.

The measurements of RNA or protein used to determine recombination efficiency or production level can be made by any suitable method known to those of skill in the art. In some examples, recombination efficiency or production level is determined by measuring an amount of functional protein expressed, for example by Western blotting. In some examples, recombination efficiency or production level is determined by measuring the RNA transcript, for example using two probe based quantitative real-time PCR. For example, the first assay spans a sequence fully contained in the 3′ exonic coding sequence (labelled 3′ probe). The second assay spans the junction between the 5′ and the 3′ exonic coding sequence (labelled junction probe). Reconstitution efficiency can be calculated as the ratio of (junction probe count)/(3′ probe count). “Reconstitution efficiency,” “recombination efficiency,” and “splicing efficiency” are used interchangeably herein.

The level of expression of a reconstituted protein, or the level of successful editing of a nucleic acid sequence (and, e.g., production of an encoded protein) achieved using the compositions and methods provided herein may be evaluated based on an indirect measurement, e.g., level of a representative marker, surrogate, or functional indicator. Any such marker known to those of skill in the art may be used for evaluating the level of a protein reconstituted or edited according to the present disclosure, and compared with a control accordingly. For example, the formation of Dystrophin-glycoprotein complex (DGC) or a subcomplex thereof can be representative of restoration of dystrophin function. See, e.g., Gao and McNally, 2015, “The Dystrophin Complex: structure, function and implications for therapy” Compr Physiol. 2015 5(3): 1223-39, and Omairi, et al., 2019, “Regulation of the dystrophin associated glycoprotein complex composition by the metabolic properties of muscle fibres,” Scientific Reports (2019) 9:2770, each |incorporated herein by reference in its entirety.

In some examples, a dimerization domain is about 20 to about 1000 nt, or about 50 to about 160 nt, or about 50 to about 500 nt, or about 50 to 1000 nt, wherein reconstitution efficiency results in production of an effective amount of correctly joined RNA or full-length nucleic acid editing protein. In some examples, a dimerization domain is about 50 to about 160 nt, wherein reconstitution efficiency results in production of an effective amount of correctly joined RNA or full-length nucleic acid editing protein.

Achieving efficient recombination between multiple RNA molecules allows for packaging and delivery of transgenes into AAVs, which exceed the packaging limit of a single AAV. AAV packaging limits represent a major hurdle for gene therapy approaches for diseases caused by the absence/defect of large genes. One application of this system is expression of a nucleic acid editing protein and one or more gRNAs specific for the target gene, using viral vectors with restricted packaging capacity. Disease and genes include but are not limited to (Disease (gene, OMIM gene identifier)): 1) Duchenne muscular dystrophy and Becker muscular dystrophy (dystrophin, OMIM:300377); 2) Dysferlinopathies (Dysferlin, OMIM:603009); 3) Cystic fibrosis (CFTR, OMIM:602421); 4) Usher's Syndrome 1B (Myosin VILA, OMIM:276903); 5) Stargardt disease 1 (ABCA4, OMIM:601691); 6) Hemophilia A (Coagulation Factor VIII, OMIM:300841); 7) Von Willebrand disease (von Willebrand Factor, OMIM:613160); 8) Marfan Syndrome (Fibrillin 1, OMIM:134797); 9) Von Recklinghausen disease (neurofibromatosis-1, OMIM:162200), and hearing loss (OTOF, OMIM: 603681). In some embodiments, the target nucleic acid is in a gene that (when wild-type) encodes a protein selected from Dystrophin; Dysferlin; Myosin VIIA; Fibrillin 1; Neurofibromatosis-1; β-globin chain of hemoglobin; Clotting factor I; Clotting factor II; Clotting factor II; Clotting factor IV; Clotting factor V; Clotting factor VI; Clotting factor VII; Clotting factor VIII; Clotting factor IX; Clotting factor X; Clotting factor XI; Clotting factor XII; Clotting factor XIII; HBAI; HBA2; HBB; HBD; von Willebrand factor; MTHFR; FANCA; FANCC; FANCD2; FANCG; FANCJ; ADAMTS13; Factor V Leiden Prothrombin; IL-2RG, JAK3, IL-2 receptor gamma chain; IIr4 receptor gamma chain; IL-7 receptor gamma chain; IL-9 receptor gamma chain; IL-15 receptor gamma chain; IL-21 receptor gamma chain; RAG1; RAG2; CXCR4; IL7 receptor; ADA; PNP; WAS; CYBA, CYBB, NCF1, NCF2, NCF4; Beta-2 integrin; C-C chemokine receptor type 5 (CCR5), MSRB1; CSCR4; P17; PSIP1; CCR5; DMD; G6Pase; CEP290; ABCA4; MAGT1; arylsulfatase A (ARSA); ABCD1; IDS; IDUA; IDUA; SGSH; NAGLU; HGSNAT; GNS; GALNS; GLB1; ARSB; GUSB; HYAL1; MAN2B1; SMPD1; NPC1; NPC2; CFTR; PKD-1; PDK-2; PDK-3; HEXA; GBA; IHIT; NF-1; NF2; APOB; LDLR; LDLRAPI; PCSK9; BCR-ABL; ASXL1; RUNX2; EPHA1; PD-1; Androgen receptor; E6; E7; CD; NGF; ARSA; MBP; WASP; AADC; CLN2; ASPA; GAN; MT-ND4; SGSH; SUMF1; GAD; NTRN; TH; CHI; GDNF; GAA; SMN; and thymidine kinase. Others are provided in Tables 1-4. Expression of the nucleic acid editing protein, and for example or more gRNAs specific for the target nucleic acid, can be expressed using the disclosed systems provided herein, for example to treat genomic point mutations or activate or overexpress genes. Delivery of a nucleic acid editing protein can be achieved by splitting it into multiple fragments using the approach provided herein.

Additional applications of the disclosed methods and systems include intersectional gene delivery for targeted gene expression. One can make use of differential infection/expression patterns of two viruses encoding a fragmented gene. The reconstituted protein will get expressed in an overlapping population of cells that represents the intersection of what either virus would express in on its own. Examples for such an application may include: (1) delivery of two halves (or three thirds, or other portions) of a protein using retrogradely transported viral vectors from two (or more) projection targets to label bifurcating dual projection neurons, (2) delivery of one fragment under the control of a promoter that is active in population A and the second fragment from a promoter active in population B to specifically tag/manipulate the A∪B population, (3) delivery of the first half of a protein with a viral vector that has a tropism for population A and the second half with a viral vector that has a tropism for population B to specifically tag/manipulate the A∪B population. Or, combinations of these approaches.

In one example the dimerization domains are aptamer sequences, for example to facilitate dimerization in the presence of a (a) small molecular trigger recognized by the aptamers, or a (b) protein that is present in the cell binding to the two halves and therefore stimulating dimerization.

In some embodiments the RNA-RNA interactions necessary for end-joining can be controlled positively or negatively by other nucleotides such as (a) an antisense oligonucleotide sequence with homology to the two halves (ssDNA triggered dimerization). In such an example, an antisense oligonucleotide having a complementary sequence to both halves bridges the two molecules together, thus facilitating spliceosome mediated recombination of the two molecules, (b) an antisense oligonucleotide sequence with homology to one of the two joining-RNAs could occlude RNA-dimerization of the two molecules and serve as an off-switch for gene expression, or (c) an endogenous cellular RNA with homology to the two halves (RNA triggered dimerization). In such an example, a cellular RNA (e.g., mRNA or retroelement) having a complementary sequence to both halves bridges the two molecules together, thus facilitating spliceosome mediated recombination of the two molecules.

These molecule, protein, or RNA mediated interactions allow for controllable/fine tuned gene expression levels: Through titrating in molecules that interact with the binding domains (e.g., antisense oligonucleotides, small molecules, endogenous cellular RNAs), dimerization efficiency between the two halves can be modulated to regulate expression levels independent of promoter activity. Such an installment can be used if a narrow range of protein expression levels are needed.

### III. Systems

Provided herein is a system that can be used to recombine two or more RNA molecules, such as at least two, at least three, at least four, or at least five different RNA molecules (such as 2. 3, 4, 5, 6, 7, 8, 9 or different RNA molecules) using synthetic introns containing dimerization sequences. Unlike fragmentation and reconstitution of two fragments at the protein level, the disclosed approach does not require extensive protein engineering to find a suitable split point. Reconstitution on an RNA level allows for seamless joining of two fragments of a protein. The disclosed methods and systems allow for large genes (and corresponding proteins), such as those greater than about 4.5 kb, at least 5 kb, at least 5.5 kb, at least 6 kb, at least kb, at least 8 kb, at least 8 kb, at least 10 kb, at least 13.5 kb, or at least 18 kb, to be divided into two or more fragments or portions, which can each be introduced into a cell or subject via separate vectors, such as multiple AAV. In some examples, the disclosed methods and systems allow for nucleic acid editing genes (and corresponding proteins, such as a Cas nuclease), such as those at least greater than about 2 kb, at least 2.5 kb, at least 3 kb, at least 3.5 kb, or at least 4 kb, to be divided into two or more fragments or portions, which can each be introduced into a cell or subject via separate vectors, such as multiple AAV, wherein such vectors can further include one or more gRNA coding sequences specific for one or more target nucleic acid molecules (e.g., specific for one or more target sites to be edited, such as deletion, insertion, or substitution of one or more nucleotides or ribonucleotides). In some examples, multiple copies of the same one or more gRNA coding sequences are present, for example to increase the number of gRNA molecules expressed from the vector. In one example, the system includes two portions for recombining two RNA molecules, for example wherein the target protein is encoded by at least about 4500 nt to about 9000 nt, such as 4000 nt to 5000 nt. In one example, the system includes three portions for recombining three RNA molecules, for example wherein the nucleic acid editing protein is encoded by up to about 13,500 nt, such as about 2000 nt to about 13,500 nt or 3000 nt to 5000 nt. In one example, the system includes four portions for recombining four RNA molecules, for example wherein the nucleic acid editing protein is encoded by up to about 18,000 nt, such as about 2000 nt to about 18,000 nt or 2000 nt to 5000 nt. This helps to overcome the limited space available in vectors. In some examples, an endogenous promoter length limits the capability of its corresponding gene to be expressed in an AAV. In some examples, a coding sequence length limits its capability to be expressed in an AAV. In some examples, an endogenous promoter length and its coding sequence length limits their capability to be expressed together in an AAV. The disclosed systems can be used to express such long sequences that have been previously difficult to express in AAV. The disclosed systems can also be used to express numerous copies of one or more gRNAs, for example in combination with a nucleic acid editing protein, as the amount of gRNAs can be rate limiting. In some examples, the disclosed DNAs and systems express at least 2 gRNAs, at least 3 gRNAs, at least 4 gRNAs, at least 5 gRNAs, at least 10 gRNAs, at least 20 gRNAs, at least 50 gRNAs, at least 75 gRNAs, at least 100 gRNAs, at least 200 gRNAs, at least 500 gRNAs, or at least 1000 gRNAs, wherein the gRNAs can target the same nucleic acid molecule and the same target site, target the same nucleic acid molecule and two or more target sites within the same target nucleic acid molecule, target two or more different nucleic acid molecules (such as 1 or more target sites within each different target nucleic acid molecule), or combinations thereof.

In some examples, the one or more gRNAs target a nucleic acid molecule, such as a gene, associated with disease, such as a monogenic disease, recessive genetic disease, a disease caused by a mutation in a gene. Examples of such diseases include, but are not limited to, hemophilia A (caused by mutations in the F8 gene, 7 kb coding region, also referred to as Coagulation Factor VIII), hemophilia B (caused by mutations in the F9 gene), Duchenne muscular dystrophy (caused by mutations in the dystrophin gene, 11 kb coding region), sickle cell anima (caused by mutation in beta globin domain of hemoglobin, which has a promoter of about 3.5 kb), Stargardt disease (caused by mutations in the ABCA4 gene, 6.9 kb coding region), Usher syndrome (caused by a mutation in MYO7A, 7 kb coding region, resulting in hearing loss and visual impairment). In one example, the gene is one caused by a point mutation in a gene.

In one example, the one or more gRNAs target a nucleic acid molecule, such as a gene, to treat a disease, such as a cancer, such as a cancer of the breast, lung, prostate, liver, kidney, brain, bone, ovary, uterus, skin, or colon.

In some examples, an RNA sequence encoding the target nucleic acid editor and used in the disclosed methods and systems are codon optimized for expression in a target organism or cell, such as codon optimized for expression in a human, canine, pig, feline, mouse, or rat cell. Thus, in some examples, the RNA coding sequence includes preferred codons (e.g., does not include rare codons with low utilization). Codon optimization can be performed by identifying abundant tRNA levels in the target organism or cells. In some examples, an RNA sequence encoding the protein is de-enriched for cryptic splice donor and acceptor sites to maximize an RNA recombination reaction.

In some examples, a nucleic acid editing protein is divided into two portions, such as about two equal halves (or other proportions, such as portion A expressing about ⅓ and portion B expressing about ⅔, or portion A expressing about ¼ and portion B expressing about ¾, etc.). However, it is not required that each portion be the same number of nucleotides (or encode the same number of amino acids). In such an example, the method can use two synthetic nucleic acid molecules (e.g., RNA or DNA encoding such RNA), one which includes a coding sequence for an N-terminal portion of the protein, and another which includes a coding sequence for a C-terminal portion of the protein. Based on this foundation, one skilled in the art will appreciate that in addition to dividing a protein into two fragments or portions, nucleic acid editing proteins can be divided or split into more than two fragments, such as three fragments. The design principle of the intronic sequences of three RNA molecules is similar to that of the two, but instead a different pair of dimerization domains for one of the two junctions is utilized. Thus, for example, an N-terminal protein coding sequence is followed by an intronic sequence with a specific binding domain (e.g., first dimerization sequence), the middle coding sequence includes an intronic sequence with a complementary sequence to the first dimerization sequence (second dimerization sequence). The middle coding fragment is followed by another intronic fragment with another dimerization sequence (third dimerization sequence, different from the second dimerization sequence). The third fragment includes the C-terminal coding sequence of the protein, and includes an intronic region with a dimerization sequence (fourth dimerization sequence) complementary to the third dimerization sequence. In the use of more than one middle portion, the two middle portions may be referred to as a middle portion and a first middle portion, or as a first middle portion and a second middle portion, or as a first middle portion, a second middle portion and a third middle portion, etc., in a way understood to distinguish the respective portions.

In one example, a nucleic acid editing protein is divided into an N-terminal portion and a C-terminal portion (e.g., divided in roughly half, or unequal apportionment, such as ⅓ and ⅔ or ¼ and ¾), which can be reconstituted using the disclosed systems and methods. Referring to FIGS. 6A and 6G, in such an example, the system includes at least two synthetic nucleic acid molecules 110, 150. Each nucleic acid molecule 110, 150 can be composed of DNA or RNA (if RNA, corresponding promoters 112, 152 are absent). In some examples, each of 110, 150 is about at least 100 nucleotides/ribonucleotides (nt) in length, such as at least 200, at least 300, at least 500, at least 1000, at least 2000, at least 3000, at least 4000, at least 5000, at least 6000, at least 7000, at least 8000 nt, at least 10,000 nt, such as 200 to 10,000 nt, 200 to 8000 nt, 500 to 5000 nt, 800 to 3000 nt, 1000 to 300 nt, or 200 to 1000 nt. The molecules 110, 150 can include natural and/or non-natural nucleotides or ribonucleotides.

Molecule 110 is the 5′-located molecule of the system, as it includes a splice donor 116. In embodiments where molecule 110 is DNA, it includes a promoter 112 operably linked to a sequence encoding an RNA molecule, the RNA molecule comprising from 5′ to 3′: a coding sequence for an N-terminal portion of the target protein 114, wherein the coding sequence for an N-terminal portion of the target protein 114 comprises a splice junction at a 3′-end of the target protein coding sequence, SD 116, optional DISE 118, optional ISE 120, dimerization domain 122, and optional polyadenylation sequence 124. Any promoter 112 (or enhancer) can be used, such as one that utilizes RNA polymerase II, such as a constitutive or inducible promoter. In some examples, promoter 112 is a tissue-specific promoter, such as one constitutively active in muscle tissue (such as skeletal or cardiac), optical tissue (such as retinal tissue), inner ear tissue, liver tissue, pancreatic tissue, lung tissue, skin tissue, bone, or kidney tissue. In some examples, promoter 112 is a cell-specific promoter, such as one constitutively active in a cancer cell, or a normal cell. In some examples, promoter 112 is an endogenous promoter of the protein expressed, and in some example is long (e.g., at least 2500 nt, at least 3000 nt, at least 4000 nt, at least 5000 nt, or at least 7500 nt). In some examples, promoter 112 is at least about 50 nucleotides (nt) in length, such as at least 100, at least 200, at least 300, at least 500, at least 1000, at least 2000, at least 3000, at least 4000, at least 5000, at least 6000, at least 7000, at least 8000 nt, at least 9000 nt, or at least 10,000 nt, such as 50 to 10,000 nt, 100 to 5000 nt, 500 to 5000 nt, or 50 to 1000 nt in length. In some examples, molecule 110 is DNA, and is at least 200, at least 300, at least 500, at least 800, at least 1000, at least 2000, at least 3000, at least 4000, at least 5000, at least 6000, at least 7000, or at least 8000 nt, such as 200 to 10,000 nt, 200 to 8000 nt, 500 to 5000 nt, 800 to 3000 nt, 1000 to 300 nt, or 200 to 1000 nt in length. As shown in FIG. 6F, in embodiments where molecule 110 is RNA, for example after transcription of the DNA into RNA, molecule 110 does not include promoter 112, and 114 is the RNA encoded by the coding sequence for an N-terminal portion of the nucleic acid editing protein. In some examples, molecule 110 is RNA, does not include promoter 112, and is at least 200, at least 300, at least 500, at least 1000, at least 2000, at least 3000, at least 4000, at least 5000, at least 6000, at least 7000, or at least 8000 nt, such as 200 to 10,000 nt, 200 to 8000 nt, 500 to 5000 nt, 800 to 3000 nt, 1000 to 300 nt, or 200 to 1000 nt in length. The molecule 110 (with or without promoter 112) can include natural and/or non-natural nucleotides or ribonucleotides.

The splice junction around the 3′ end of the N-terminal coding sequence (or RNA sequence encoded thereby) 114 can match the consensus sequence found in the target cell or organism into which molecules 110, 150 are introduced. In humans the splice junction sequence is AG (adenine-guanine) or UG (uracil-guanine) at position −1 and −2 of the 5′ splice site for U2-dependent introns or AG, UG, CU (cytosine-uracil), or UU for U12-dependent introns. Thus, in some examples, the splice junction is 2 nt in length, and the 3′ end of the N-terminal coding portion 114 is AG, UG, CU or UU. In some examples a DNA molecule encoding a portion of a nucleic acid editing protein comprises sequences that encode parts of multiple splice junctions, e.g., at the 3′ end of the DNA molecule encoding the N-terminal portion of the nucleic acid editing protein, and at the 5′ end of the DNA molecule encoding the C-terminal portion of the nucleic acid editing protein.

The remaining 3′-terminal portion of molecule 110 is intronic, 130. In some examples, intronic sequence 130 is about at least 10 nt, such as at least 20 nt, at least 50 nt, at least 100 nt, at least 250 nt, at least 250 nt, at least 300 nt, at least 400 nt, or at least 500 nt in length, such as 20 to 500, 20 to 250, 20 to 100, 50 to 100, or 50 to 200 nt in length. Immediately following N-terminal coding sequence (or RNA encoded thereby) 114 is a splice donor (SD) 116 (such as a SD consensus sequence, such as a SD human consensus sequence). Thus SD 116 of intronic sequence 130 is 3′ to N-terminal coding sequence 114. SD 116 forms a recognition sequence for the spliceosome components to bind to the RNA molecule. The sequence of SD 116 can be a SD consensus sequence found in the target cell or organism into which molecules 110, 150 are introduced. In some examples, SD 116 is at least 2 nt, such as at least 5 nt, or at least 10 nt in length, such as 2 to 10, 2 to 8, 2 to 5 or 5 to 10 nt. The SD 116 can be used to recruit U2 or U12 dependent splicing machinery. In one example, U2 dependent splicing is used in human cells, and the SD 116 sequence includes or is GUAAGUAUU. In one example, U12 dependent splicing is used in human cells, and the SD 116 sequence includes or is AUAUCCUUUUUA (SEQ ID NO: 137) or GUAUCCUUUUUA (SEQ ID NO: 138). Throughout, it is understood that RNA sequences can be described using nucleotides A, G, U and C, and that DNA sequences can be described using nucleotides A, G, T and C. It is also understood that sequences described herein as comprised by a DNA molecule that is transcribed to form one or more RNA molecules, and sequences described as comprised by an RNA molecule that may be translated (i.e., a protein coding sequence) or not translated (e.g., a gRNA), are the sequences with the intended function as can be recognized by one of skill in the art. For example, a gRNA sequence present in a DNA molecule of the disclosure has a sequence that is functional following its transcription. As another example, a target protein coding sequence present in a DNA molecule of the disclosure has a sequence that can be translated into the target protein from mRNA that is transcribed from the DNA. A sequence referred to as encoded by the DNA or RNA molecule, or comprised by the DNA or RNA molecule, is one that results in the intended, functional product, as understood by one of skill in the art reading the present disclosure.

Intronic sequence 130 optionally includes one or both of a set of splicing enhancer sequences referred to as downstream intronic splice enhancer (DISE) 118 and intronic splice enhancer (ISE) 120, which stimulate action (e.g., increase activity) of the spliceosome. In some examples, intronic sequence 130 includes at least two splicing enhancer sequences, such as at least 3, at least 4, or at least 5 splicing enhancer sequences. Exemplary splicing enhancer sequences include DISE 118 and ISE 120. In some examples, inclusion of one or more splicing enhancer sequences 118, 120 in intronic sequence 130 increases splicing efficiency by at least 20%, at least 30%, at least 40%, at least 50%, at least 75%, at least 80%, at least 90% or at least 95%. Exemplary splicing enhancer sequences that can be used are provided in SEQ ID NOS: 26-136, 151, and 152, as well as GGGTTT, GGTGGT, TTPGGG, GAGGGG, GGTATT, GTAACG, GGGGGTAGG, GGAGGGTIT, GGGTGGTGT TTCAT, CCATIT, TITTAAA, TGCAT, TGCATG, TGTGTT, CTAAC, TCTCT, TCTGT, TCTIT, TGCATG, CTAAC, CTGCT, TAACC, AGCTT, TTCATTA, GTTAG, TTTTGC, ACTAAT, ATGTIT, CTCTG, GGG, GGG(N)2-4GGG, TGGG, YCAY, UGCAUG, or 3×(G₃₋₆N₁₋₇). In some examples, if DISE 118 is present, can be at least 3 nt, at least 4 nt, at least 5 nt, at least 10 nt, at least 25 nt, at least 50 nt, at least 75 nt, or at least 100 nt in length, such as 3 to 10, 3 to 11, 4 to 11, 5 to 11, 10 to 50, 5 to 100, 10 to 25, 10 to 20, or 20 to 75 nt, the sequence of DISE 118 is or comprises CUCUUUCUUUTCCAUGGGUUGGCU (SEQ ID NO: 134), TGCATG, CTAAC, CTGCT, TAACC, AGCIT, TTCATTA, GTTAG, TITrGC, ACTAAT, ATGTTT or CTCTG. In some examples, if ISE 120 is present, it can be about at least 3 nt, at least 4 nt, at least 5 nt, at least 10 nt, such as at least 20 nt, at least 25 nt, at least 30 nt, at least 40 nt, or at least 50 nt in length, such as 3 to 10, 3 to 11, 4 to 11, 5 to 11, to 50, 20 to 25, 10 to 25, 10 to 20, or 20 to 40 nt in length. In one example, the sequence of ISE 120 is or comprises GGCUGAGGGAAGGACUGUCCUGGG (SEQ ID NO: 135), GGGUUAUGGGACC (SEQ ID NO: 136), TTCAT, CCATIT, TTITAAA, TGCAT, TGCATG, TGTGTT, CTAAC, TCTCT, TCTGT, or TCTIT. In some examples, intronic sequence 130 includes at least two, at least 3, or at least 4 ISEs 120. In some examples, ISE 120 is or comprises at least one sequence having at least 80%, at least 90%, at least 95%, at least 96%, at least 97%, at least 98%, at least 99% or 100% sequence identity to SEQ NO: 173, 174, 175, 176, 177, 178, 179, 180, 182, 183, 184, 185, 186, 187, 188, 189, 190, 191, 192, 193, 194, 195, 196, 199, 200, 201, 202, or 203, such as at least 2, at least 3 of such sequences, such as 1, 2, 3, 4 or 5 of such sequences. In some examples, DISE 118 is or comprises at least one sequence having at least 80%, at least 90%, at least 95%, at least 96%, at least 97%, at least 98%, at least 99% or 100% sequence identity to SEQ NO: 173, 174, 175, 176, 177, 178, 179, 180, 182, 183, 184, 185, 186, 187, 188, 189, 190, 191, 192, 193, 194, 195, 196, 199, 200, 201, 202, or 203, such as at least 2, at least 3 of such sequences, such as 1, 2, 3, 4 or of such sequences.

The SD 116 (and if present also enhancer sequences 118, 120) is followed 3′ by a dimerization domain 122 used to bring the N-terminal coding sequence (or RNA encoded thereby) 114, and C-terminal coding sequence 154 to be combined, together. Intronic sequence 130 portion of molecule 110 can optionally include at the 3′-end a polyadenylation site 124, which terminates transcription of that fragment. In some examples, polyadenylation sequence 124 is a polyA sequence of at least 15 As, such as 15 to 30 or to 20 As.

In some examples, first dimerization domain 122 (and second dimerization domain 154 of molecule 150) includes a plurality of unpaired nucleotides (that is, unpaired within the structure of the molecule 110 itself). Having unpaired nucleotides in the dimerization domain allows the 5′ (or first) dimerization domain 122 and the 3′ (or second) dimerization domain 154 to interact through base pairing. Through this interaction, molecules 110 and 150 are kept in proximity which prompts the spliceosome to recombine the two molecules by joining the N-terminal coding region (or RNA encoded thereby) 114 and the C terminal coding region (or RNA encoded thereby) 164.

In one example, dimerization domain 122 (and 154) includes “hypodiverse sequences,” which contain a limited diversity of nucleotides and are thus unlikely to form stem loops with themselves in the secondary structure of each molecule 110, 150. Such a hypodiverse dimerization domain 122 (and 154) can be a relatively open configuration, independent of the sequences of the DNA encoding the N- and C-terminus of the protein (or RNA encoded thereby) 114, 164. This allows the nucleotides of the first dimerization domain 122 to be available to form base pairs with the corresponding second dimerization domain 154 of molecule 150, allowing subsequent joining of the N-terminal coding sequence (or RNA encoded thereby) 114 and C-terminal coding sequence (or RNA encoded thereby) 164. In some examples, first and second dimerization domain 122, 154 includes hypodiverse sequences interspersed with sequences that can form a stem, which results in local RNA loops that are open and available for basepairing in the absence of pseudoknot formation (FIG. 6B). Exemplary hypodiverse sequences include a repeated series of Us (such as 30 to 500 Us), a repeated series of As (such as 30 to 500 As), a repeated series of Gs (such as 30 to 500 Gs), a repeated series of Cs (such as 30 to 500 Cs), a mixture containing only As and Gs (such as 30 to 500 As and Gs, e.g., AAAGAAGGAA ( . . . ) (SEQ ID NO: 149) which can be repeated), a mixture containing only Cs and Us (such as 30 to 500 Cs and Us, e.g., CUUUCUUUUCUU ( . . . ) (SEQ ID NO: 150) which can be repeated). Other exemplary hypodiverse sequences include complementary sequences that form helices flanked by hypodiverse sequences.

In some examples, first and second dimerization domain 122, 154 only include purines or only include pyrimidines. In one example, the first dimerization domain 122 only includes purines, while the second dimerization domain 154 only includes pyrimidines. In another example, the first dimerization domain 122 only includes pyrimidines, while the second dimerization domain 154 only includes purines.

Due to the inability of purines to pair with themselves (and pyrimidines likewise) these stretches of RNA have an open predicted structure.

In some examples, first and second dimerization domain 122, 154 do not include cryptic splice acceptors that could compete with RNA recombination, such as sequences similar to the splice donor consensus sequence NNNAGGUNNNN (SEQ ID NO: 151) or NNNUGGUNNNN (SEQ ID NO: 152) (wherein N refers to any nucleotide). In some examples, first dimerization domain 122 is no more than 1000 nt, such as no more than 750 nt, or more than 500 nt, such as 6 to 1000 nt, 10 to 1000 nt, 20 to 1000 nt, 30 to 1000 nt, 30 to 750 nt, 30 to 500 nt, 50 to 500 nt, 50 to 100 nt, or 100 to 250 nt. In some examples, first dimerization domain 122 is greater than 50 nt, such as at least 51 nt, at least 100 nt, at least 150 nt, at least 161 nt, or at least 170 nt, such as 51 to 159 nt, 51 to 150 nt, 51 to 120 nt, 51 to 100 nt, or 51 to 70 nt. In some examples, first dimerization domain 122 is greater than 160 nt, such as at least 161 nt, at least 170 nt, at least 180 nt, at least 200 nt, at least 300 nt, at least 400 nt, at least 500 nt, at least 600 nt, at least 700 nt, at least 800 nt, at least 900 nt, or at least 1000 nt, such as 161 to 100 nt, 161 to 500 nt, 161 to 300 nt, 161 to 200 nt, or 161 to 170 nt. In some examples, first dimerization domain 122 is less than 50 nt, such 6 to 49 nt, 6 to 45 nt, 6 to 40 nt, 6 to 30 nt, 6 to 20 nt, or 6 to 10 nt.

In some examples, a dimerization domain is 20 to 160 nt, 50-500 nt, or 500-1000 nt. In some examples, a dimerization domain is about 20 nt to about 160 nt. In some examples, a dimerization domain is about 20 nt to about 40 nt, about 20 nt to about 50 nt, about 20 nt to about 70 nt, about 20 nt to about 90 nt, about 20 nt to about 100 nt, about 20 nt to about 110 nt, about 20 nt to about 120 nt, about 20 nt to about 130 nt, about 20 nt to about 140 nt, about 20 nt to about 150 nt, about 20 nt to about 160 nt, about 40 nt to about 50 nt, about 40 nt to about 70 nt, about 40 nt to about 90 nt, about 40 nt to about 100 nt, about 40 nt to about 110 nt, about 40 nt to about 120 nt, about 40 nt to about 130 nt, about 40 nt to about 140 nt, about 40 nt to about 150 nt, about 40 nt to about 160 nt, about 50 nt to about 70 nt, about 50 nt to about 90 nt, about 50 nt to about 100 nt, about 50 nt to about 110 nt, about 50 nt to about 120 nt, about 50 nt to about 130 nt, about 50 nt to about 140 nt, about 50 nt to about 150 nt, about 50 nt to about 160 nt, about 70 nt to about 90 nt, about 70 nt to about 100 nt, about 70 nt to about 110 nt, about 70 nt to about 120 nt, about 70 nt to about 130 nt, about 70 nt to about 140 nt, about 70 nt to about 150 nt, about 70 nt to about 160 nt, about 90 nt to about 100 nt, about 90 nt to about 110 nt, about 90 nt to about 120 nt, about 90 nt to about 130 nt, about 90 nt to about 140 nt, about 90 nt to about 150 nt, about 90 nt to about 160 nt, about 100 nt to about 110 nt, about 100 nt to about 120 nt, about 100 nt to about 130 nt, about 100 nt to about 140 nt, about 100 nt to about 150 nt, about 100 nt to about 160 nt, about 110 nt to about 120 nt, about 110 nt to about 130 nt, about 110 nt to about 140 nt, about 110 nt to about 150 nt, about 110 nt to about 160 nt, about 120 nt to about 130 nt, about 120 nt to about 140 nt, about 120 nt to about 150 nt, about 120 nt to about 160 nt, about 130 nt to about 140 nt, about 130 nt to about 150 nt, about 130 nt to about 160 nt, about 140 nt to about 150 nt, about 140 nt to about 160 nt, or about 150 nt to about 160 nt. In some examples, a dimerization domain is about 20 nt, about nt, about 50 nt, about 70 nt, about 90 nt, about 100 nt, about 110 nt, about 120 nt, about 130 nt, about 140 nt, about 150 nt, or about 160 nt. In some examples, a dimerization domain is at least about 20 nt, about 40 nt, about 50 nt, about 70 nt, about 90 nt, about 100 nt, about 110 nt, about 120 nt, about 130 nt, about 140 nt, or about 150 nt. In some examples, a dimerization domain is at most about 40 nt, about 50 nt, about 70 nt, about 90 nt, about 100 nt, about 110 nt, about 120 nt, about 130 nt, about 140 nt, about 150 nt, or about 160 nt.

In some examples, a dimerization domain is about 50 nt to about 500 nt. In some examples, a dimerization domain is about 50 nt to about 100 nt, about 50 nt to about 150 nt, about 50 nt to about 200 nt, about 50 nt to about 250 nt, about 50 nt to about 300 nt, about 50 nt to about 350 nt, about 50 nt to about 400 nt, about 50 nt to about 500 nt, about 100 nt to about 150 nt, about 100 nt to about 200 nt, about 100 nt to about 250 nt, about 100 nt to about 300 nt, about 100 nt to about 350 nt, about 100 nt to about 400 nt, about 100 nt to about 500 nt, about 150 nt to about 200 nt, about 150 nt to about 250 nt, about 150 nt to about 300 nt, about 150 nt to about 350 nt, about 150 nt to about 400 nt, about 150 nt to about 500 nt, about 200 nt to about 250 nt, about 200 nt to about 300 nt, about 200 nt to about 350 nt, about 200 nt to about 400 nt, about 200 nt to about 500 nt, about 250 nt to about 300 nt, about 250 nt to about 350 nt, about 250 nt to about 400 nt, about 250 nt to about 500 nt, about 300 nt to about 350 nt, about 300 nt to about 400 nt, about 300 nt to about 500 nt, about 350 nt to about 400 nt, about 350 nt to about 500 nt, or about 400 nt to about 500 nt. In some examples, a dimerization domain is about 50 nt, about 100 nt, about 150 nt, about 200 nt, about 250 nt, about 300 nt, about 350 nt, about 400 nt, or about 500 nt. In some examples, a dimerization domain is at least about 50 nt, about 100 nt, about 150 nt, about 200 nt, about 250 nt, about 300 nt, about 350 nt, or about 400 nt. In some examples, a dimerization domain is at most about 100 nt, about 150 nt, about 200 nt, about 250 nt, about 300 nt, about 350 nt, about 400 nt, or about 500 nt.

In some examples, the sequence of first and second dimerization domains 122 and 154 are determined by in silico structure prediction screening (e.g., RNA folding structure prediction is used to screen a library of possible dimerization domain sequences; sequences with a large proportion of unpaired nucleotides in both the dimerization domain and the corresponding anti-dimerization domain are selected), hypodiverse nucleotide design (e.g., dimerization domain designed to include a stretch of hypodiverse sequence, such as a repeat sequence of only U, only A, only C, only G, only R (G and A), or only Y (U and C), the sequence cannot fold onto itself), or empirical screening (e.g., a library of dimerization domains and corresponding anti-dimerization domains are synthesized and screened for maximal recombination efficiency).

In some examples, the sequence of first and second dimerization domains 122, 154 are designed to contain complementary RNA hairpin structures (also called stem loops) that can form strong kissing loop interactions with their counter parts. In some examples, kissing loops are used when three or more dimerization domains are used to join three or more portions of a coding sequence, such as four or more or five or more dimerization domains, such as 3, 4, 5, 6, 7, 8, 9 or 10 dimerization domains (e.g., FIG. 6E).

Each hairpin loop (or stem loop) of a kissing loop is composed of at least two complementary sequences (e.g., form a stem) separated by a region of non-complementary sequence (e.g., form a loop). In some examples, a dimerization domain can be composed of 1 or more (such as at least 2, at least 3, at least 4, or at least 5, such as 2, 3, 4, 5, 6, 7, 8, 9, 10, 11, 12, 13, 14, 15, 16, 17, 18, 19, or 20) loops. In some examples with multiple loops, all or some of the loops can be repeated. In some examples with multiple loops, all or some loops can be different In some examples, each complementary sequence is about 4 to 100 nt, which are separated by a loop of about 3 to 20 nt. Base-pairing between the two complementary sequences results in a helix (or stem), for example of at least 4 bp, at least 5 bp, at least 10 bp, at least 20 bp, at least 30 bp, at least 40 bp, at least 50 bp, at least 75 bp, at least 90 bp, or at least 100 bp, such as 4 to 100 bp, 5 to 75 bp, or to 50 bp. In some examples, the loop portion is at least 3 nt, at least 5 nt, at least 10 nt, at least 15 nt, or at least 20 nt, such as 3 to 20 nt, 5 to 15 nt or 5 to 10 nt, wherein the loop is not base paired. Complementary sequences between two hairpin loops result in base pairing, and generation of a kissing loop/kissing stem loop interaction. In some examples, the complementary sequences between the two hairpin loops occurs between at least 3 nucleotides of one loop with at least 3 nucleotides of a second loop, such as at least 4, at least 5, at least 6, at least 7, at least 8, at least 9, at least 10, at least 11, at least 12, at least 13, at least 14, at least 15, at least 16, at least 17, at least 19, or at least 20 nt (such as 3, 4, 5, 6, 7, 8, 9, 10, 11, 12, 13, 14, 15, 16, 17, 18, 19 or 20) of the first loop, with at least 4, at least 5, at least 6, at least 7, at least 8, at least 9, at least 10, at least 11, at least 12, at least 13, at least 14, at least 15, at least 16, at least 17, at least 19, or at least 20 nt (such as 3, 4, 5, 6, 7, 8, 9, 10, 11, 12, 13, 14, 15, 16, 17, 18, 19 or 20) of the second loop. In some examples, the complementary sequences between the two hairpin loops occurs between at least 15%, at least 20%, at least 30%, at least 40%, at least 50%, at least 60%, at least 70%, at least 80%, at least 90%, at least 95%, or 100% of the total loop sequence.

In some instances, the stems of the kissing loops are chosen to base pair in trans between the two RNA molecules. In such an example, after forming a kissing loop interaction of one hairpin loop on one molecule with another hairpin loop on a second molecule, the respective stem (or helix) regions of the initial hairpin loops can base pair in trans between the two RNA molecules through strand replacement/invasion and extended duplex formation. In some examples, within the initial loop sequence, up to about 85% of nucleotides can remain unpaired after extended duplex formation (e.g., about 15% of the nt are paired between the two loops). In some examples, the kissing loop is based on the HIV-1 DIS loop (SEQ ID NOS: 139 and 140, FIG. 17A), and includes two A nucleotides on the 5′ side of 6 nucleotides of complementary sequence, followed by one A nucleotide on the 3′ side (e.g., AANNNNNNA where N can be any of A, U, G, or C). In some examples, the kissing loop is based on the HIV-2 kissing loop dimerization domain (SEQ ID NOS: 141 and 142, FIG. 17B), and includes a G and an A nucleotide on the 5′ side of six nucleotides of complementary sequence followed by three A nucleotides on the 3′ side (e.g., GANNNNNNAAA (SEQ ID NO: 153) where N can be A, U, G, or C).

In one configuration, extended duplex formation is favored by inclusion of mismatches in the initial stems that result in higher percentage of matching in the extended duplex. Thus, in some examples, the helix or stem region of a hairpin loop can contain up to 30% of base pairs that are not paired initially (e.g., no more than 30%, no more than 20%, no more than 15%, no more than 10%, no more than 5%, or no more than 1%, such as 1 to 30%, 5 to 30%, 10 to 30%, or 25 to 30% of base pairs are not paired initially). These regions of non-pairing can form bulges, mismatches, or internal loops.

In addition to an interaction of two hairpin loops (kissing loop interaction), other forms of loop interactions can be utilized for the first and second dimerization domains 122, 154. In one example the loops are bulges, where one strand of a base paired helix contains one or more nucleotides that bulge out from the stem structure. Exemplary bulges are at least 1 nt, at least 2 nt, at least 3 nt, at least 4 nt, at least 5 nt, at least 10 nt or at least 20 nt, such as 1 to 20 nt, 1 to 15 nt, 1 to 10 nt, or 5 to 10 nt, or 1, 2, 3, 4, 5, 6, 7, 8, 9, 10, 11, 12, 13, 14, 15, 16, 17, 18, 19 or 20 nt. In one example the loops are internal loops, for example, where 1 or more nucleotides in a helix are mismatched, resulting in a helix interrupted by an internal loop at the positions of mismatch. In some examples the helix is at least 4 nt on each of the strands (e.g., at least 5 nt, at least 10 nt, at least 20 nt, at least 30 nt, at least 40 nt, at least 50 nt, at least 75 nt, at least 90 nt, or at least 100 nt, such as 4 to 100 nt, 5 to 75 nt, or 10 to 50 nt. such as 4 to 100 nt), on either side of the internal loop that is at least 1 nt (e.g., at least 2 nt, at least 3 nt, at least 4 nt, at least 5 nt, at least 10 nt or at least 20 nt, such as 1 to 20 nt, 1 to 15 nt, 1 to 10 nt, or 5 to 10 nt, or 1, 2, 3, 4, 5, 6, 7, 8, 9, 10, 11, 12, 13, 14, 15, 16, 17, 18, 19 or 20 nt on each of the strands). In one example the loops are multi-branched loops, wherein three helices or stems from a triangle with one or more unpaired nucleotides connecting the three helices. In some examples, each of the helices is at least 4 bp (e.g., at least 5 bp, at least 10 bp, at least 20 bp, at least 30 bp, at least 40 bp, at least 50 bp, at least 75 bp, at least 90 bp, or at least 100 bp, such as 4 to 100 bp, 5 to 75 bp, or 10 to 50 bp), and the unpaired nucleotides that form the triangle are at least 3 nt (e.g., at least 4 nt, at least 5 nt, at least 10 nt, at least 20, at least 15, at least 30, at least 40, at least 50, or at least 60 nt, such as 3 to 60 nt, 3 to 30 nt, 3 to 25 nt, or 5 to 20 nt, such as 1, 2, 3, 4, 5, 6, 7, 8, 9, 10, 11, 12, 13, 14, 15, 16, 17, 18, 19, 2, 25, 30, 35, 40, 45, 50, 55 or 60 nucleotides). A kissing interaction can occur between any two of these types of loops (e.g., between two or more binding domains that each include one or more helices). In some examples, helices within one dimerization domain (e.g., first dimerization domain 122) have a direct counterpart in the other binding domain (e.g., second dimerization domain 154) to allow for extended duplex formation after initial loop kissing interaction. In some examples, dimerization domains containing helices to generate loops, form a single kissing stem loop upon interaction between the two or more dimerization domains (e.g., 122, 154 of FIG. 6A). In some examples, dimerization domains containing helices form multiple loops for kissing loop interactions upon interaction between the two or more dimerization domains (e.g., 122, 154 of FIG. 6A). In some examples, one or more dimerization domains (e.g., 122 of FIG. 6A) contain helices destabilized by the inclusion of bulges, single base bulges, mismatches or internal loops, or G-U wobble pairs, but match to the other binding domain (e.g., 154 of FIG. 6A), to favor extended duplex formation after initial kissing/pairing. In some examples, one or more dimerization domains (e.g., 122 of FIGS. 6A, 6G) contain destabilized helices, which when stabilized (e.g., theophylline switch kissing loop) expose a loop that can interact with a second dimerization domain (e.g., 122 of FIGS. 6A, 6G) via loop-loop interactions (e.g., kissing/pairing).

In some examples these stem loops contain at least 10 nt, such as at least 20 nt, at least 25 nt, at least 50 nt, at least 75 nt, or at least 100 nt in length, such as 10 to 50, 20 to 25, 10 to 100, 10 to 20, or 20 to 40 nt in length. Each dimerization domain can contain at least 1 individual stem loop, such as at least 2, at least 5, at least 10, at least 15, or at least 20, such as 1 to 20, 2 to 5 or 1 to 10 individual stem loops.

In some examples, 3 to 10 portions of a coding sequence are joined by 2 to 9 kissing loops, e.g., 3 portions are joined by 2 kissing loops, 4 portions are joined by 3 kissing loops, etc., wherein each of the 2 to 9 kissing loops are different. In some examples, a kissing loop comprises multiple stem loops, e.g., 2 to 20 stem loops. In some examples, each of the multiple stem loops in the kissing loop are the same. In some examples, each of the multiple stem loops in the kissing loop are different. In some examples, a dimerization domain comprises 1 to 20 stem loops. In some examples, a dimerization domain comprises 1 stem loop to 20 stem loops. In some examples, a dimerization domain comprises 1 stem loop to 2 stem loops, 1 stem loop to 3 stem loops, 1 stem loop to 4 stem loops, 1 stem loop to 5 stem loops, 1 stem loop to 6 stem loops, 1 stem loop to 7 stem loops, 1 stem loop to 8 stem loops, 1 stem loop to 9 stem loops, 1 stem loop to 10 stem loops, 1 stem loop to 15 stem loops, 1 stem loop to 20 stem loops, 2 stem loops to 3 stem loops, 2 stem loops to 4 stem loops, 2 stem loops to 5 stem loops, 2 stem loops to 6 stem loops, 2 stem loops to 7 stem loops, 2 stem loops to 8 stem loops, 2 stem loops to 9 stem loops, 2 stem loops to 10 stem loops, 2 stem loops to 15 stem loops, 2 stem loops to 20 stem loops, 3 stem loops to 4 stem loops, 3 stem loops to 5 stem loops, 3 stem loops to 6 stem loops, 3 stem loops to 7 stem loops, 3 stem loops to 8 stem loops, 3 stem loops to 9 stem loops, 3 stem loops to 10 stem loops, 3 stem loops to 15 stem loops, 3 stem loops to 20 stem loops, 4 stem loops to 5 stem loops, 4 stem loops to 6 stem loops, 4 stem loops to 7 stem loops, 4 stem loops to 8 stem loops, 4 stem loops to 9 stem loops, 4 stem loops to 10 stem loops, 4 stem loops to 15 stem loops, 4 stem loops to 20 stem loops, 5 stem loops to 6 stem loops, 5 stem loops to 7 stem loops, 5 stem loops to 8 stem loops, 5 stem loops to 9 stem loops, 5 stem loops to 10 stem loops, 5 stem loops to 15 stem loops, 5 stem loops to 20 stem loops, 6 stem loops to 7 stem loops, 6 stem loops to 8 stem loops, 6 stem loops to 9 stem loops, 6 stem loops to 10 stem loops, 6 stem loops to 15 stem loops, 6 stem loops to 20 stem loops, 7 stem loops to 8 stem loops, 7 stem loops to 9 stem loops, 7 stem loops to 10 stem loops, 7 stem loops to 15 stem loops, 7 stem loops to 20 stem loops, 8 stem loops to 9 stem loops, 8 stem loops to 10 stem loops, 8 stem loops to 15 stem loops, 8 stem loops to 20 stem loops, 9 stem loops to 10 stem loops, 9 stem loops to stem loops, 9 stem loops to 20 stem loops, 10 stem loops to 15 stem loops, 10 stem loops to 20 stem loops, or 15 stem loops to 20 stem loops. In some examples, a dimerization domain comprises 1 stem loop, 2 stem loops, 3 stem loops, 4 stem loops, 5 stem loops, 6 stem loops, 7 stem loops, 8 stem loops, 9 stem loops, stem loops, 15 stem loops, or 20 stem loops. In some examples, a dimerization domain comprises at least 1 stemloop, 2 stemloops, 3 stemloops, 4 stem loops, 5 stemloops, 6 stemloops, 7 stemloops, 8 stem loops, 9 stem loops, 10 stem loops, or 15 stem loops. In some examples, a dimerization domain comprises at most 2 stem loops, 3 stem loops, 4 stem loops, 5 stem loops, 6 stem loops, 7 stem loops, 8 stem loops, 9 stem loops, 10 stem loops, 15 stem loops, or 20 stem loops.

Other mechanisms can be used to allow the two or more dimerization domains (e.g., 122, 154 of FIGS. 6A, 6G) to bind or interact with one another sufficient for recombination of the coding sequences to occur. In some examples, the two or more dimerization domains (e.g., 122, 154 of FIGS. 6A, 6G) are nucleic acid aptamers (such as RNA aptamers) that can interact with one another, for example through a non-base pairing interaction, or can bind to a common molecule (e.g., protein, ATP, metal ion, co-factor, or synthetic ligand). In some examples, two or more dimerization domains (e.g. 122, 154 of FIGS. 6A, 6G) do not hybridize to one another, but can both (or all) hybridize to the same bridge nucleic acid molecule. In some examples, such a bridge nucleic acid molecule can be exogenously provided to the cells, tissues, or organism. In some examples, such a bridge nucleic acid molecule can be a DNA or RNA sequence inside the cell, such as a transcript or genomic locus. In some examples, the two or more dimerization domains (e.g., 122, 154 of FIGS. 6A, 6G) are sequences that can interact with one another, for example through a non-base pairing interaction.

Molecule 150 is the 3′-located molecule, and includes a splice acceptor (SA) 162 and a second dimerization domain 154. In embodiments where molecule 150 is DNA, it includes a second promoter 152 followed by intronic sequence 170. Promoter 152 can be is operably linked to intronic sequence 170. Any promoter 152 can be used, such as a constitutive or inducible promoter. In some examples, promoter 152 is a tissue-specific promoter, such as one constitutively active in muscle tissue (such as skeletal or cardiac), optical tissue (such as retinal tissue), inner ear tissue, liver tissue, pancreatic tissue, lung tissue, skin tissue, bone, or kidney tissue. In some examples, promoter 112 is a cell-specific promoter, such as one constitutively active in a cancer cell, or a normal cell. In some examples, promoter 112 is an endogenous promoter of the target protein expressed, and in some examples is long (e.g., at least 2500 nt, at least 3000 nt, at least 4000 nt, at least 5000 nt, or at least 7500 nt). In some examples, promoter 112 is at least about 50 nucleotides (nt) in length, such as at least 100, at least 200, at least 300, at least 500, at least 1000, at least 2000, at least 3000, at least 4000, at least 5000, at least 6000, at least 7000, at least 8000 nt, at least 9000 nt, or at least 10,000 nt, such as 50 to 10,000 nt, 100 to 5000 nt, 500 to 5000 nt, or 50 to 1000 nt in length. In some examples promoter 112 and promoter 152 are the same promoter. In other examples, promoter 112 and promoter 152 are the different promoters. In some examples, molecule 150 is DNA, and is at least 200, at least 300, at least 500, at least 1000, at least 2000, at least 3000, at least 4000, at least 5000, at least 6000, at least 7000, or at least 8000 nt, such as 200 to 10,000 nt, 200 to 8000 nt, 500 to 5000 nt, or 200 to 1000 nt in length. As shown in FIG. 6F, in embodiments where molecule 150 is RNA, for example after expression of the DNA into RNA, molecule 150 no longer includes promoter 152, and 164 is the RNA encoded by the coding sequence for a C-terminal portion of the nucleic acid editing protein. In some examples, molecule 150 is RNA, does not include promoter 152, and is at least 200, at least 300, at least 500, at least 1000, at least 2000, at least 3000, at least 4000, at least 5000, at least 6000, at least 7000, or at least 8000 nt, such as 200 to 10,000 nt, 200 to 8000 nt, 500 to 5000 nt, or 200 to 1000 nt in length. Molecule 150 (with or without promoter 152) can include natural and/or non-natural nucleotides or ribonucleotides.

The intronic sequence 170 includes a second dimerization domain 154, optional ISE 156, branching point 158, polypyrimidine tract 160, followed by a splice acceptor sequence 162. In some examples, intronic sequence 130 is about at least 10 nt, such as at least 20 nt, at least 30 nt, at least 50 nt, at least 100 nt, at least 250 nt, at least 250 nt, at least 300 nt, at least 400 nt, or at least 500 nt in length, such as 20 to 500, 20 to 250, 20 to 100, 50 to 100, 30 to 500, or 50 to 200 nt in length.

Second dimerization domain 154 has a sequence that is the reverse complement of first dimerization domain 122 sequence of molecule 110. Thus, same design features and considerations of first dimerization domain 122 discussed above also apply to second dimerization domain 154. For example, in some examples the second dimerization domain 154 contains a stem loop that can form a kissing loop interaction the first dimerization domain 122. In some examples, second dimerization domain 154 does not include cryptic splice acceptors (e.g., NNNAGGUNNN; SEQ ID NO: 143) that could compete with RNA recombination.

In some example, second dimerization domain 154 has a hypodiverse sequence. In some examples, second dimerization domain 154 is no more than 1000 nt, such as no more than 750 nt, or more than 500 nt, such as to 1000 nt, 30 to 750 nt, 30 to 500 nt, 50 to 500 nt, 50 to 100 nt, or 100 to 250 nt. In some examples, second dimerization domain 154 is greater than 50 nt, such as at least 51 nt, at least 100 nt, at least 150 nt, at least 161 nt, or at least 170 nt, such as 51 to 159 nt, 51 to 150 nt, 51 to 120 nt, 51 to 100 nt, or 51 to 70 nt. In some examples, second dimerization domain 154 is greater than 160 nt, such as at least 161 nt, at least 170 nt, at least 180 nt, at least 200 nt, at least 300 nt, at least 400 nt, at least 500 nt, at least 600 nt, at least 700 nt, at least 800 nt, at least 900 nt, or at least 1000 nt, such as 161 to 100 nt, 161 to 500 nt, 161 to 300 nt, 161 to 200 nt, or 161 to 170 nt. In some examples, second dimerization domain 154 is less than 50 nt, such 6 to 49 nt, 6 to 45 nt, 6 to 40 nt, 6 to 30 nt, 6 to 20 nt, or 6 to 10 nt, 3′- to second dimerization domain 154 is an optional ISE 156, branch point sequence 158 (such as a branch point consensus sequence), polypyrimidine tract 160, followed by a splice acceptor sequence 162. ISE 156, like ISE 120 and DISE 118 of molecule 110, stimulates the spliceosome to catalyze the recombination reaction. In some examples, intronic sequence 150 includes at least two ISE 156, such as at least 3, at least 4, or at least 5 ISEs 156. Exemplary splicing enhancer sequences include ISE 156. In some examples, inclusion of one or more splicing enhancer sequences 156 in intronic sequence 150 increases recombination or splicing efficiency by at least 10%, at least 20%, at least 30%, at least 40%, or at least 50%. Exemplary splicing enhancer sequences that can be used are provided in SEQ ID NOS: 26-136, 151, and 152, as well as GGGTTT, GGTGGT, TITGGG, GAGGGG, GGTATT, GTAACG, GGGGGTAGG, GGAGGGTIT, GGGTGGTGT TTCAT, CCATT, TITTAAA, TGCAT, TGCATG, TGTGTT, CTAAC, TCTCT, TCTGT, TCTIT, TGCATG, CTAAC, CTGCT, TAACC, AGCIT, TTCATTA, GTTAG, TITTGC, ACTAAT, ATGTIT, CTCTG, GGG, GGG(N)2-4GGG, TGGG, YCAY, UGCAUG, or 3×(G₃₋₆N₁₋₇). In some examples, if ISE 156 is present, it can be about least 3 nt, at least 4 nt, at least 5 nt, at least nt, such as at least 20 nt, at least 25 nt, at least 30 nt, at least 40 nt, or at least 50 nt in length, such as 3 to 10, 3 to 11, 4 to 11, 5 to 11, 10 to 50, 20 to 25, 10 to 25, 10 to 20, or 20 to 40 nt in length. In one example, the sequence of ISE 156 is or comprises GGCUGAGGGAAGGACUGUCCUGGG (SEQ ID NO: 135), GGGUUAUGGGACC (SEQ ID NO: 136), TTCAT, CCATIT, TITTAAA, TGCAT, TGCATG, TGTGTT, CTAAC, TCTCT, TCTGT, or TCTIT. In some examples ISE 120 and ISE 156 are the same sequence. In other examples, ISE 120 and ISE 156 are the different sequences. 3′- to second dimerization domain 154 (and ISE 156 if present) is branch point sequence 158 (such as a branch point consensus sequence), a polypyrimidine tract 160, followed by a splice acceptor sequence 162 (such as a splice acceptor consensus sequence). The sequence of branch point 158 is based on the consensus sequence of the species of the target cell or organism. For example, for human splicing, the consensus sequence can include or be YUNAY. Thus, a sequence that it uses can be CUAAC for U2-dependent introns, or for U12-dependent introns UUUUCCUUAACU (SEQ ID NO: 144).

Polypyrimidine tract 160 includes C, U, or both C and U nucleotides, such as CnUy, wherein n+y is greater than or equal to 10 nucleotides, and can include nucleotides −3 to −22 relative to the 3′-splice junction. In some examples, polypyrimidine tract 160 includes at least 80% Y nucleotides (i.e., U, C, or both U and C). In some examples, polypyrimidine tract 160 is a polyC or polyU sequence. In some examples, polypyrimidine tract 160 is a polyU sequence of at least 15 Us, such as 15 to 30 or 15 to 20 Us. Branch point 158 and polypyrimidine tract 160 are essential splicing components. The sequence of SA 162 can be based on the consensus sequence of the species of the target cell or organism. For example, in humans, the SA sequence can be AG in positions −1 and −2 relative to the 3′-splice site for U2-dependent introns and AC or AG for U12-dependent introns. Thus, in some examples, SA 162 can be 2 nt in length, such as AG or AC.

Immediately following SA 162 is an exonic sequence which includes a DNA sequence encoding a C-terminal portion of a target protein 164 having a splice junction at its 5′end. The splice junction at the 5′end of DNA sequence encoding a C-terminal portion of a nucleic acid editing protein 164, that can match the consensus sequence found in the target cell or organism into which molecules 110, 150 are introduced. In some examples splice junction can be GA or GU at positon+1 and +2 of the 3′ splice site for U2-dependent introns or GU or AU for U12-dependent introns. Thus, in some examples, the splice junction is 2 nt in length, and the 5′ end of the C-terminal coding portion 164 is GA, GU, or AU.

The exonic sequence following intronic portion 170 of molecule 150 includes a second coding portion (e.g., half) of the nucleic acid editing protein, e.g., the C terminal fragment 164, and optional polyadenylation sequence 166. Thus, molecule 150 includes sequence 164 encoding a C-terminal portion of a nucleic acid editing protein. The 3′-end of molecule 150 optionally includes a polyadenylation sequence 166, which promotes the assembly of the spliceosome. In some examples, polyadenylation sequence 166 is a polyA sequence of at least 15 As, such as 15 to 30 or 15 to 20 As. In some examples polyadenylation sequence 166 and polyadenylation sequence 124 are the same sequence. In other examples, polyadenylation sequence 166 and polyadenylation sequence 124 are the different sequences.

In some examples, the N-terminal coding region 114 and/or the C terminal coding region 164 is a native coding sequence. For example, the coding sequence is one that is found in the cell or organism into which the disclosed system is introduced. (e.g., a human coding sequence when introduced into a human cell or subject). In some examples, the N-terminal coding region 114 and/or the C terminal coding region 164 is codon optimized relative to a native coding sequence, for example to maximize tRNA availability, or to de-enrich for cryptic splice sites (e.g., to reduce or avoid incorrect splicing and promote the correct junction formation). In some examples, a portion of the N-terminal coding region 114 and/or the C terminal coding region 164 is codon optimized relative to a native coding sequence, for example the about 200 nt adjacent to each junction (e.g., the 3′-end of 114, and the 5′end of 164) can be codon optimized or altered to contain exonic splice enhancer sites (ESE) (which would bind SR proteins). For example, the coding sequence can be one not found in the cell or organism into which the disclosed system is introduced (e.g., a human coding sequence when introduced into a mouse cell or subject).

In some examples, the N-terminal coding region 114 and/or the C terminal coding region 164 include an intron that is either natural or synthetic in nature and contains both a splice donor and acceptor site. For example, an intron embedded inside the to the coding sequence to be expressed can be included upstream (e.g., about 200 nt upstream) of sequence 116, inside the N-terminal coding region 114, an intron embedded inside the coding sequence to be expressed can be included downstream (e.g., about 200 nt downstream) of the sequence 162 and inside the C-terminal coding region 164, or both. Inclusion of such introns can be used to stimulate splicing machinery attachment to the trans-splicing intron donor and acceptor. In some examples, such (stimulatory-) introns could be derived from the host in which 110 and 150 are expressed. In some examples, such (stimulatory-) introns could be derived from other organisms, or viral in origin, or synthetic in origin.

In some examples, inclusion of a sequence to stabilize the molecule 150 (e.g., placed between 164 and 166 in the 3′ untranslated region of 150 in FIG. 6A) can increase expression efficiency of the recombined product by at least 25%, at least 30%, at least 40%, at least 50%, at least 60%, or at least 75%, such as 25 to 95%, 25 to 75%, 25 to 60%, 25 to 50%, 40 to 95%, 40 to 60%, or 50 to 60%. In some examples, woodchuck post-transcriptional regulatory element (WPRE) or truncations thereof (e.g., WPRE3) are included in the 3′-UTR as a stabilizing element to enhance recombined product expression efficiency. In some example a WPRE sequence has at least 80%, at least 85%, at least 90%, at least 95%, or 100% sequence identity to nt 1093 to 1684 of GenBank accession no. J04514 or to the 247 bp sequence of WPRE3.

As shown in FIG. 6G, in some examples, the system or DNA used to express the nucleic acid editing protein can further includes one or more gRNA coding sequences 140, 141, 171, 172. Each gRNA includes a first portion that specifically hybridizes to a target nucleic acid molecule (which in some examples is at least 15 nt, at least 16 nt, at least 17 nt, at least 18 nt, at least 19 nt, at least 20 nt, at least 25 nt, at least 30 nt, at least 35 nt, at least 40 nt, such as 15-50 nt, 15-40 nt, 15-30 nt, 15-25 nt, 28-32 nt, 25-35 nt, 17-24 nt, or 17-20 nt, such as about 20 nt), and a second portion that binds to the nucleic acid editing protein (which in some examples is at least 20 nt, at least 30 nt, at least 40 nt, at least 50 nt, at least 60 nt, at least 70 nt, at least 75 nt, at least 80 nt, at least 90 nt, or at least 100 nt, such as 20-200 nt, 25-150 nt, 30-100 nt, 15-30 nt, 30-75 nt, or 75-100 nt). In some examples, the portion of the gRNA that specifically hybridizes to a target nucleic acid molecule has a GC content of about 40-80%. In some examples, one gRNA coding sequence 140, 141, 171, and 172 is at least about 60 nt, at least about 75 nt, at least about 80 nt, at least about 90 nt, at least about 100 nt, at least about 110 nt, or at least about 120 nt, such as 60-300 nt, 60-200 nt, 80-200 nt, 90-200 nt, 100-150 nt, or 100-120 nt. Expression of each gRNA 140, 141, 171, 172 can be driven by a promoter 142, 143, 173, 174, respectively, operably linked to the gRNA. In one example, each promoter 142, 143, 173, 174 is the same. However, the promoter for each guide nucleic acid molecule 140, 141, 171, 172 can differ. In some examples, the promoter is a polymerase III promoter, such as a human or mouse U6 or H1 promoter. Once expressed, a resulting gRNA will form a complex with the expressed nucleic acid editor, allowing editing of a nucleic acid molecule to which the gRNA hybridizes. Thus, in some examples, the system or RNA compositions are as shown in FIG. 6C, showing the joined N-terminal coding sequence 114 and C-terminal coding sequence 164, wherein the system or RNA could include one or more additional RNA molecules, namely on or more gRNA molecules (expressed from each gRNA 140, 141, 171, 172). In some examples, a system or RNA composition of FIG. 6C includes the two RNA molecules shown hybridized to one another, and further includes one or more of (a) a third RNA molecule comprising at least one first gRNA specific for a first target nucleic acid molecule, wherein the at least one first gRNA directs the nucleic acid editing protein to a target editing site on the first target nucleic acid molecule; (b), a fourth RNA molecule comprising at least one second gRNA specific for (i) the first target nucleic acid molecule, wherein the at least one second gRNA directs the nucleic acid editing protein to the same or different target editing site on the first nucleic acid molecule, or (i) a second target nucleic acid molecule, wherein the at least one second gRNA directs the nucleic acid editing protein to a target editing site on the second target nucleic acid molecule; (c) a fifth RNA molecule comprising at least one third gRNA specific for (i) the first target nucleic acid molecule, wherein the at least one third gRNA directs the nucleic acid editing protein to the same or different target editing site on the first nucleic acid molecule as the first and second gRNA, (ii) the second target nucleic acid molecule, wherein the at least one third gRNA directs the nucleic acid editing protein to the same or different target editing site on the second nucleic acid molecule as the second gRNA, or (iii) a third target nucleic acid molecule, wherein the at least one third gRNA directs the nucleic acid editing protein to a target editing site on the third target nucleic acid molecule; and (d) a sixth RNA molecule comprising at least one fourth gRNA specific for (i) the first target nucleic acid molecule, wherein the at least one fourth gRNA directs the nucleic acid editing protein to the same or different target editing site on the first nucleic acid molecule as the first, second, and third gRNA, (ii) the second target nucleic acid molecule, wherein the at least one fourth gRNA directs the nucleic acid editing protein to the same or different target editing site on the second nucleic acid molecule as the second and third gRNA, (iii) the third target nucleic acid molecule, wherein the at least one fourth gRNA directs the nucleic acid editing protein to the same or different target editing site on the third target nucleic acid molecule as the third gRNA or (iv) a fourth target nucleic acid molecule, wherein the at least one fourth gRNA directs the nucleic acid editing protein to a target editing site on the fourth target nucleic acid molecule.

Although the one or more gRNA coding sequences 140, 141, 171, 172 are shown in FIG. 6G near the 5′ and 3′-ends of each molecule 110, 150, the gRNA coding sequences 140, 141, 171, 172 can be located elsewhere in within each molecule 110, 150. In addition, the promoter-gRNA coding sequences can be in the forward or reverse orientation relative to the direction of expression of the N- and C-terminal coding sequences 114, 164. Thus, in some examples molecule 110 includes (a) one or more promoter/gRNA coding sequences (e.g., 142/140) upstream of the N terminal coding sequence 114, (b) one or more promoter/gRNA coding sequences (e.g., 143/141) downstream of the N terminal coding sequence 114, or (c) both (a) and (b), and in some examples molecule 140 includes (d) one or more promoter/gRNA coding sequences (e.g., 173/171) upstream of the C terminal coding sequence 164, (e) one or more promoter/gRNA coding sequences (e.g., 174/172) downstream of the C terminal coding sequence 164, or both (d) and (e).

FIG. 6G exemplifies the presence of four gRNAs 140, 141, 171, 172. However, a system or DNA can include fewer or more than four gRNAs. In some examples, a system, DNA, or RNA includes at least 1, at least 2, at least 3, at least 4, at least 5, at least 6, at least 7, at least 8, at least 9, at least 10, at least 20, at least 25, at least 50, at least 100, at least 500, or at least 500 gRNA sequences or coding sequences. The gRNAs of a system, DNA or RNA composition can (1) target the same nucleic acid molecule (e.g., gene) and the same target editing site of the target nucleic acid; (2) target the same nucleic acid molecule (e.g., gene) and different target editing sites of the target nucleic acid; (3) target different nucleic acid molecules (e.g., genes); or (4) any combination thereof. In some examples, one or more of gRNA coding sequence 140, 141, 171, 172 is a cassette including two or more gRNAs, allowing expression of a greater amount of gRNAs. In some examples, a cassette encodes at least two gRNAs, at least 3 gRNAs, at least 4 gRNAs, at least 5 gRNAs, at least 10 gRNAs, at least 20 gRNAs, at least 50 gRNAs, at least 100 gRNAs, or at least 500 gRNAs, wherein the sequence of each gRNA coding sequence in a cassette can be the same or different.

FIG. 6G exemplifies four gRNAs coding sequences 140, 141, 171, 172 as part of molecules 110, 150. However, in one example, one or more gRNA coding sequences 140, 141, 171, 172 are not part of molecules 110, 150, but are instead expressed from one or more separate DNA synthetic molecules, such as one or more other vectors. Thus, in some system and DNA embodiments, additional synthetic DNA molecules are provided (e.g., in addition to molecules 110, 150), which encode one or more gRNAs. In one example, the system or DNA further includes at least one additional synthetic DNA encoding a first gRNA operably linked to a promoter, such as a synthetic DNA encoding one or more gRNAs operably linked to a promoter.

FIG. 6G exemplifies the presence of 5′- and 3′-end parvovirus inverted terminal repeats (ITR) 176, 177, 178, 179 on each molecule 110, 150. A parvovirus 1TR includes an original of replication and is used to help AAV replicate. Such 1TRs can be used with a parvoviral packaging plasmid, such as AAV.

However, sequences 176, 177, 178, 179 are optional. In one example, a parvovirus ITR is an adeno-associated virus (AAV) ITR.

As shown in FIG. 6C, interaction and hybridization (base pairing) between first dimerization domain 122 of molecule 110 and second dimerization domain 154 of molecule 150, allows the spliceosome components to recombine N-terminal coding sequence 114 and C-terminal coding sequence 164.

Specifically, the 3′ end of the N terminal protein coding sequence 114 is fused to the 5′ end of the C terminal protein sequence 164 as a seamless junction between the two portions. In examples where the system or DNA composition includes gRNA coding sequences, such as exemplified in FIG. 6G, the same interaction and hybridization occurs to recombine N-terminal coding sequence 114 and C-terminal coding sequence 164, allowing for expression of a functional nucleic acid editing protein. The gRNAs expressed from 140, 141, 171, and 172 (whether as part of molecules 110, 150 or expressed from one or more different DNA molecules) would be present in the cell where expression occurred and form a complex with the expressed nucleic acid editing protein (for example via interactions with the tracrRNA and DR sequences of the gRNAs) to permit nucleic acid editing of a target nucleic acid molecule.

FIGS. 6D and 6H show a schematic of a system wherein a nucleic acid editing protein is divided into three portions, an N-terminal, middle, and C-terminal portion (wherein each portion can be similar or different in size). One skilled in the art will appreciate that a nucleic acid editing protein can thus be divided into any number of desired segments or portions, and an appropriate number of molecules designed using the information provided herein. In such an example, the system includes at least three synthetic nucleic acid molecules 110, 200, and 150, wherein molecule 110 includes molecule 114 which encodes the N-terminal portion of the protein, molecule 200 includes molecule 216 which encodes the middle portion of the protein, and molecule 150 includes molecule 164 which encodes the C-terminal portion of the protein. Each nucleic acid molecule 110, 200, 150 can be composed of DNA, and following translation, can be RNA with promoters 112, 202, 152 absent. In some examples, each of 110, 200, 150 (with or without promoters 112, 202, 152) is at least about 100 nucleotides/ribonucleotides (nt) in length, such as at least 200, at least 300, at least 500, at least 1000, at least 2000, at least 3000, at least 4000, at least 5000, at least 6000, at least 7000, or at least 8000 nt, such as 200 to 10,000 nt, 200 to 8000 nt, 500 to 5000 nt, or 200 to 1000 nt. The molecules 110, 150, 200 (with or without promoters 112, 202, 152) can include natural and/or non-natural nucleotides or ribonucleotides. In addition to using two (or more) orthogonal dimerization domains, one of the two introns can be a U2-type intron and the second intron can be a U12-type intron. Splice donor and acceptors of U2 and U12 dependent introns show minimal cross reactivity since the consensus recognition sequences between the two types of introns are different. Both strategies (i.e., the orthogonal dimerization domains, and the U2 vs U12 type introns) promote recombination of the three fragments in the correct order (e.g., to avoid the first fragment to directly join up to the last fragment and to avoid the middle fragment circularizing onto itself).

Molecule 110 of FIGS. 6D and 6H includes the same features disclosed above for FIGS. 1A and 6G, namely a promoter 112 operably linked to a sequence encoding an RNA molecule, the RNA molecule comprising from 5′ to 3′: a coding sequence for an N-terminal portion of the target protein 114, wherein the coding sequence for an N-terminal portion of the target protein 114 comprises a splice junction at a 3′-end of the target protein coding sequence, SD 116, optional DISE 118, optional ISE 120, dimerization domain 122, and optional polyadenylation sequence 124, but wherein first dimerization domain 122 has reverse complementary to third dimerization domain 204 of molecule 200. As shown in FIG. 6F, in embodiments where molecule 110 is RNA, for example after expression of the DNA into RNA, molecule 110 does not include promoter 112, and 114 is the RNA encoded by the coding sequence for an N-terminal portion of the target protein. Molecule 110 (with or without promoter 112) can include natural and/or non-natural nucleotides or ribonucleotides.

Molecule 150 of FIGS. 6D and 6H includes the same features disclosed above for FIGS. 1A and 6G, namely promoter 152 operably linked to a sequence encoding an RNA molecule, the RNA molecule comprising from 5′ to 3′: a second dimerization domain 154, optional ISE 156, a branch point sequence 158, a polypyrimidine tract 160, a splice acceptor (SA) 162; and a coding sequence for a C-terminal portion of the target protein 164, wherein the coding sequence for the C-terminal portion of the target protein comprises a splice junction at a 5′-end of the target protein coding sequence, and optionally polyadenylation sequence 166. The second dimerization domain 154 has reverse complementary to fourth dimerization domain 226 of molecule 200. Molecule 150 (with or without promoter 152) can include natural and/or non-natural nucleotides or ribonucleotides.

Molecule 200 allows for the joining of the N- and C-terminal coding regions 114, 164, by providing dimerization domains having reverse complementarity to dimerization domains 122, 154 of molecule 110 and molecule 150, respectively. Molecule 200 includes features from both molecule 110 and molecule 150, including two intronic sequences 230, 240. Specifically, in embodiments where molecule 200 is DNA, molecule 220 includes promoter 210 (which can be the same or different than promoter 112 and/or 152) operably linked to a sequence encoding an RNA molecule, the RNA molecule comprising from 5′ to 3′: third dimerization domain 204 (which is the reverse complement to first dimerization domain 122 of molecule 110 in FIG. 6D), optional ISE 206, branch point 208, polypyrimidine tract 210, SA 212, a coding sequence for a middle portion of the target protein 216, wherein the coding sequence for the middle portion of the target protein 216 comprises a splice junction at a 5′-end of the target protein coding sequence and a splice junction at a 3′-end of the target protein coding sequence, SD 220, optional DISE 222, optional ISE 224, fourth dimerization domain 226 (which is the reverse complement to fourth dimerization domain 154 of molecule 150 in FIG. 6D), and optional polyadenylation sequence 228. In some examples, molecule 220 is DNA, and is at least 200, at least 300, at least 500, at least 1000, at least 2000, at least 3000, at least 4000, at least 5000, at least 6000, at least 7000, or at least 8000 nt, such as 200 to 10,000 nt, 200 to 8000 nt, 500 to 5000 nt, or 200 to 1000 nt in length. In embodiments where molecule 200 is RNA, for example after expression of the DNA into RNA, molecule 200 no longer includes promoter 202, and 216 is the RNA encoded by the coding sequence for a middle portion of the target protein. In some examples, molecule 200 is RNA, does not include promoter 202, and is at least 200, at least 300, at least 500, at least 1000, at least 2000, at least 3000, at least 4000, at least 5000, at least 6000, at least 7000, or at least 8000 nt, such as 200 to 10,000 nt, 200 to 8000 nt, 500 to 5000 nt, or 200 to 1000 nt in length. The molecule 200 (with or without promoter 202) can include natural and/or non-natural nucleotides or ribonucleotides.

As shown in FIG. 6H, in some examples, the system or DNA used to express the nucleic acid editing protein can further includes one or more gRNA coding sequences 140, 141, 171, 172, 231, 232.

Each gRNA includes a first portion that specifically hybridizes to a target nucleic acid molecule (which in some examples is at least 15 nt, at least 16 nt, at least 17 nt, at least 18 nt, at least 19 nt, at least 20 nt, at least nt, at least 30 nt, at least 35 nt, at least 40 nt, such as 15-50 nt, 15-40 nt, 15-30 nt, 15-25 nt, 28-32 nt, 25-nt, 17-24 nt, or 17-20 nt, such as about 20 nt), and a second portion that binds to the nucleic acid editing protein (which in some examples is at least 20 nt, at least 30 nt, at least 40 nt, at least 50 nt, at least 60 nt, at least 70 nt, at least 75 nt, at least 80 nt, at least 90 nt, or at least 100 nt, such as 20-200 nt, 25-150 nt, 30-100 nt, 15-30 nt, 30-75 nt, or 75-100 nt). In some examples, the portion of the gRNA that specifically hybridizes to a target nucleic acid molecule has a GC content of about 40-80%. In some examples, one gRNA coding sequence 140, 141, 171, 172, 231, 232 is at least about 60 nt, at least about 75 nt, at least about 80 nt, at least about 90 nt, at least about 100 nt, at least about 110 nt, or at least about 120 nt, such as 60-300 nt, 60-200 nt, 80-200 nt, 90-200 nt, 100-150 nt, or 100-120 nt. Expression of each gRNA 140, 141, 171, 172, 231, 232 can be driven by a promoter 142, 143, 173, 174, 233, 234 respectively, operably linked to the gRNA. In one example, each promoter 142, 143, 173, 174, 233, 234 is the same. However, the promoter for each guide nucleic acid molecule 140, 141, 171, 172, 233, 234 can differ. In some examples, promoter 142, 143, 173, 174, 233, 234 is a polymerase III promoter, such as a human or mouse U6 or H1 promoter. Once expressed, a resulting gRNA will form a complex with the expressed nucleic acid editor, allowing editing of a nucleic acid molecule to which the gRNA hybridizes. Thus, in some examples, the system or RNA compositions are as shown in FIG. 6E, showing the joined N-terminal coding sequence 114, middle coding sequence 216, and C-terminal coding sequence 164, wherein the system or RNA could include one or more additional RNA molecules, namely on or more gRNA molecules (expressed from each gRNA 140, 141, 171, 172, 231, 232). In some examples, a system or RNA composition of FIG. 6E includes the three RNA molecules shown hybridized to one another, and further includes one or more of (a) a fourth RNA molecule comprising at least one first gRNA specific for a first target nucleic acid molecule, wherein the at least one first gRNA directs the nucleic acid editing protein to a target editing site on the first target nucleic acid molecule; (b), a fifth RNA molecule comprising at least one second gRNA specific for (i) the first target nucleic acid molecule, wherein the at least one second gRNA directs the nucleic acid editing protein to the same or different target editing site on the first nucleic acid molecule, or (i) a second target nucleic acid molecule, wherein the at least one second gRNA directs the nucleic acid editing protein to a target editing site on the second target nucleic acid molecule; (c) a sixth RNA molecule comprising at least one third gRNA specific for (i) the first target nucleic acid molecule, wherein the at least one third gRNA directs the nucleic acid editing protein to the same or different target editing site on the first nucleic acid molecule as the first and second gRNA, (ii) the second target nucleic acid molecule, wherein the at least one third gRNA directs the nucleic acid editing protein to the same or different target editing site on the second nucleic acid molecule as the second gRNA, or (iii) a third target nucleic acid molecule, wherein the at least one third gRNA directs the nucleic acid editing protein to a target editing site on the third target nucleic acid molecule; (d) a seventh RNA molecule comprising at least one fourth gRNA specific for (i) the first target nucleic acid molecule, wherein the at least one fourth gRNA directs the nucleic acid editing protein to the same or different target editing site on the first nucleic acid molecule as the first, second, and third gRNA, (ii) the second target nucleic acid molecule, wherein the at least one fourth gRNA directs the nucleic acid editing protein to the same or different target editing site on the second nucleic acid molecule as the second and third gRNA, (iii) the third target nucleic acid molecule, wherein the at least one fourth gRNA directs the nucleic acid editing protein to the same or different target editing site on the third target nucleic acid molecule as the third gRNA or (iv) a fourth target nucleic acid molecule, wherein the at least one fourth gRNA directs the nucleic acid editing protein to a target editing site on the fourth target nucleic acid molecule; (e) a seventh RNA molecule comprising at least one fifth gRNA and (f) an eighth RNA molecule comprising at least one sixth gRNA.

Although the one or more gRNA coding sequences 140, 141, 171, 172, 231, 232 are shown in FIG. 6H near the 5′ and 3′-ends of each molecule 110, 150, the gRNA coding sequences 140, 141, 171, 172 can be located elsewhere in within each molecule 110, 150. In addition, the promoter-gRNA coding sequences can be in the forward or reverse orientation relative to the direction of expression of the N-terminal, middle, and C-terminal coding sequences 114, 216, 164. In addition, the promoter-gRNA coding sequences can be in the forward or reverse orientation relative to the direction of expression of the N-terminal, middle, and C-terminal coding sequences 114, 216, 164. Thus, in some examples molecule 110 includes (a) one or more promoter/gRNA coding sequences (e.g., 142/140) upstream of the N terminal coding sequence 114, (b) one or more promoter/gRNA coding sequences (e.g., 143/141) downstream of the N terminal coding sequence 114, or (c) both (a) and (b), in some examples molecule 140 includes (d) one or more promoter/gRNA coding sequences (e.g., 173/171) upstream of the C terminal coding sequence 164, (e) one or more promoter/gRNA coding sequences (e.g., 174/172) downstream of the C terminal coding sequence 164, or (f) both (d) and (e), and in some examples molecule 200 includes (g) one or more promoter/gRNA coding sequences (e.g., 233/231) upstream of the middle coding sequence 216, (h) one or more promoter/gRNA coding sequences (e.g., 234/232) downstream of the middle coding sequence 216, or (i) both (g) and (h).

FIG. 6H exemplifies the presence of six gRNAs 140, 141, 171, 172, 231, 232. However, a system or DNA can include fewer or more than six gRNAs. In some examples, a system, DNA, or RNA includes at least 1, at least 2, at least 3, at least 4, at least 5, at least 6, at least 7, at least 8, at least 9, at least 10, at least 20, at least 25, at least 50, at least 100, at least 500, or at least 500 gRNA sequences or coding sequences.

The gRNAs of a system, DNA or RNA composition can (1) target the same nucleic acid molecule (e.g., gene) and the same target editing site of the target nucleic acid; (2) target the same nucleic acid molecule (e.g., gene) and different target editing sites of the target nucleic acid; (3) target different nucleic acid molecules (e.g., genes); or (4) any combination thereof. In some examples, one or more of gRNA coding sequence 140, 141, 171, 172, 231, 232 is a cassette including two or more gRNAs, allowing expression of a greater amount of gRNAs. In some examples, a cassette encodes at least two gRNAs, at least 3 gRNAs, at least 4 gRNAs, at least 5 gRNAs, at least 10 gRNAs, at least 20 gRNAs, at least 50 gRNAs, at least 100 gRNAs, or at least 500 gRNAs, wherein the sequence of each gRNA coding sequence in a cassette can be the same or different.

FIG. 6H exemplifies four gRNAs coding sequences 140, 141, 171, 172, 231, 232 as part of molecules 110, 150, 200. However, in one example, one or more gRNA coding sequences 140, 141, 171, 172, 231, 232 are not part of molecules 110, 150, 200 but are instead expressed from one or more separate DNA synthetic molecules, such as one or more other vectors. Thus, in some system and DNA embodiments, additional synthetic DNA molecules are provided (e.g., in addition to molecules 110, 150, 200), which encode one or more gRNAs. In one example, the system or DNA further includes at least one additional synthetic DNA encoding a first gRNA operably linked to a promoter, such as a synthetic DNA encoding one or more gRNAs operably linked to a promoter.

FIG. 6H exemplifies the presence of 5′- and 3′-end parvovirus inverted terminal repeats (ITR) 176, 177, 178, 179, 235, 236 on each molecule 110, 200, 150. Such ITRs can be used with a parvoviral packaging plasmid, such as AAV. However, sequences 176, 177, 178, 179, 235, 236 are optional.

As shown in FIG. 6E, interaction and hybridization (base pairing) between first dimerization domain 122 of molecule 110 and third dimerization domain 204 of molecule 200, and interaction and hybridization (base pairing) between fourth dimerization domain 226 of molecule 200 and second dimerization domain 154 of molecule 150, allows the spliceosome components to recombine N-terminal coding sequence 114, middle coding sequence 216, and C-terminal coding sequence 164. Specifically, the 3′ end of the N terminal protein coding sequence 114 is fused to the 5′ end of the middle protein sequence 216, and the 3′ end of middle protein sequence 216, is fused to the 5′ end of the C-terminal protein sequence 164 as a seamless junction between the three portions. In examples where the system or DNA composition includes gRNA coding sequences, such as exemplified in FIG. 6H, the same interaction and hybridization occurs to recombine N-terminal coding sequence 114, middle coding sequence 216, and C-terminal coding sequence 164, allowing for expression of a functional nucleic acid editing protein. The gRNAs expressed from 140, 141, 171, 172, 231, 232 (whether as part of molecules 110, 150, 200 or expressed from one or more different DNA molecules) would be present in the cell where expression occurred and form a complex with the expressed nucleic acid editing protein (for example via interactions with the tracrRNA and DR sequences of the gRNAs) to permit nucleic acid editing of a target nucleic acid molecule.

Alternative dimerization domains are shown in FIGS. 7A-7B and 9A. That is, as an alternative to using dimerization domains that hybridize to one another (e.g., 112 to 204, 226 to 154, FIGS. 6C, 6E), in one example aptamer sequences are used. As shown in FIG. 7A, in both synthetic nucleic acid molecules 500, 600, aptamer sequences 512, 602 are used instead of the dimerization domains, and the aptamers come together via their interaction with a target (such as adenosine, dopamine, or caffeine). In such an example, the aptamer sequence 512, 602 of each molecule 500, 600 can be the same, or even be different sequences. Molecule 500 of FIG. 7A includes the same features disclosed above for molecule 110 of FIGS. 6A and 6G, which when DNA includes a promoter operably linked to a sequence encoding an RNA molecule, the RNA molecule comprising from 5′ to 3′: a coding sequence for an N-terminal portion of the nucleic acid editing protein 502, wherein the coding sequence for an N-terminal portion of the target protein 502 comprises a splice junction at a 3′-end of the nucleic acid editing protein coding sequence, SD 506, optional DISE 508, optional ISE 510, a first aptamer 512 instead of a first dimerization domain, and optional polyadenylation sequence. In embodiments when molecule 500 is RNA, for example when transcribed from the DNA molecule, molecule 500 does not include a promoter (e.g., as shown in FIG. 7A). Similarly, molecule 600 of FIG. 7A includes the same features disclosed above for molecule 150 of FIGS. 6A and 6G, which when DNA includes a promoter operably linked to a sequence encoding an RNA molecule, the RNA molecule comprising from 5′ to 3′: aptamer 602 instead of second dimerization domain 154, optional ISE 604, branch point 606, polypyrimidine tract 608, SA 610, DNA encoding a C-terminal portion of a target protein 614 with a splice junction at its 5′-end, and optional polyadenylation sequence 616. In embodiments when molecule 600 is RNA, for example when transcribed from the DNA molecule, molecule 500 does not include a promoter (e.g., as shown in FIG. 7A). Interaction of the two aptamers 512, 602, with each other or molecule 700 allows the spliceosome components to recombine N-terminal coding sequence 502 and C-terminal coding sequence 614. Specifically, the 3′ end of the N terminal protein coding sequence 502 is fused to the 5′ end of the C terminal protein sequence 614 as a seamless junction between the two portions.

Molecules 500 and 600 can include natural and/or non-natural nucleotides or ribonucleotides.

In some examples, aptamer sequences 512, 602 recognize (e.g., specifically bind) the same target 700 (FIG. 7A), or can even recognize different targets (wherein a synthetic molecule is also administered with the system provided herein, which includes each molecule specifically recognized by each aptamer, or the part of the molecule recognized by the aptamer, such as a caffeine/dopamine hybrid molecule). Exemplary targets recognized by aptamers include cellular proteins, small molecules, exogenous proteins, or an RNA molecule.

FIG. 7B shows an example similar to FIG. 7A. The dimerization domains (512, 602 FIG. 7A) recognize an RNA molecule. In the example shown in FIG. 7B, each domain recognizes a different portion of an mRNA molecule only expressed in target cells (cells where nucleic acid editing protein expression is desired), such as a cancer-specific transcript. In such an example, the coding sequences comprised by the RNAs (502, 614 of FIG. 7A) only recombine in the presence of the specific RNA molecule recognized by the dimerization domains. Here, the nucleic acid editing protein would only be expressed in cancer cells, not normal cells. Such a system allows for control of the nucleic acid editing protein expression.

FIG. 7C provides an exemplary “off-switch” example. Here, the hybridization/binding of dimerization domains 812, 902(which are reverse complements of one another) of synthetic nucleic acid molecules 800, 900 can be reduced by providing an anti-binding domain oligonucleotide (e.g., RNA or DNA) 1000 (which can be two different anti-binding domain oligonucleotides 1000, one that is the reverse complement of 812, and one that is the reverse complement of 912) that competes for the binding/hybridization. Anti-binding domain oligonucleotide 1000 can thus act as an “off-switch” for reconstitution of the protein encoded by N- and C-terminal coding portions 802 and 914, respectively. Molecule 800 of FIG. 7C includes the same features disclosed above for a molecule 110 of FIGS. 6A and 6G that is an RNA molecule (and thus lacks a promoter), which RNA molecule comprises from 5′ to 3′: a coding sequence for an N-terminal portion of the nucleic acid editing protein 802, wherein the coding sequence for an N-terminal portion of the nucleic acid editing protein 802 comprises a splice junction at a 3′-end of the target protein coding sequence, SD 806, optional DISE 808, optional ISE 810, dimerization domain 812, and optional polyadenylation sequence 814. Similarly, molecule 900 of FIG. 7C includes the same features disclosed above for a molecule 150 of FIGS. 6A and 6G that is an RNA molecule (and thus lacks a promoter), which RNA molecule comprises from 5′ to 3′: anti-dimerization domain 902, optional ISE 904, branch point 906, polypyrimidine tract 908, SA 910, RNA encoding a C-terminal portion of a nucleic acid editing protein 914, and optional polyadenylation sequence 916. The two dimerization domains 812, 902 cannot interact/hybridize to each other in the presence of the anti-binding domain oligonucleotides 1000, and therefore prevents or reduces recombination of the N-terminal coding sequence 802 and C-terminal coding sequence 914. Molecules 800 and 900 can include natural and/or non-natural nucleotides or ribonucleotides.

FIG. 9A provides an exemplary dimerization domain that uses kissing loop interactions instead of reverse complementary sequence hybridization for dimerization. Kissing loop interactions are formed when the bases in the loops of two RNA hairpins form interacting pairs between two RNA molecules. The molecule on the left hand side labelled with n-yfp represents an RNA molecule that encodes the n-terminal fragment of yfp, linked to a synthetic intron that contains a splice donor site, a downstream intronic splicing enhancer element, and two intronic splicing enhancer elements. The dimerization domain this molecule contains three RNA hairpin loops that each are composed of a stem (where the RNA hybridizes onto itself) and a loop (in which the RNA is not hybridized to itself). In this example, the dimerization domain contains three stem and loop elements (also referred to as hairpin loops) and is referred to as a trimodal kissing loop dimerization domain. The molecule on the right hand side labelled with c-yfp represents an RNA molecule that encodes the c terminal portion of yfp. From 5′ to 3′ this molecule is composed of a trimodal kissing loop dimerization domain that contains a set of three hairpin loops. The loop portions can form kissing loop interactions with the corresponding loops on the complementary n-yfp molecule. The trimodal kissing loop dimerization domain is followed by a synthetic intron sequence that contains three intronic splicing enhancer sequences, a branch point sequence, a polypyrimidine tract, and a splice acceptor site. The synthetic intron sequence is followed by the c-terminal yfp coding sequence, which is followed by a 3′ untranslated region that contains a poly adenylation signal. At the top of the figure, a representative 3-dimensional rendering of a kissing loop interaction is shown. This rendering illustrates how the kinked form of the hairpin loop exposes the loop residues towards the outside which renders them available for the kissing loop interaction.

Upon association of the two molecules, the spliceosome mediates a trans-splicing reaction which results in the joining of the n-terminal and the c-terminal ypf coding sequence which then allows for expression of the full-length fluorescent protein.

Although FIGS. 6A-6C, 6F, 6G, 7A, 7B, 7C and 9A show embodiments where a system uses two synthetic nucleic acid molecules are used (i.e., the nucleic acid editing protein coding sequence is split between two synthetic nucleic acid molecules), one skilled in the art will appreciate that such embodiments can be used similarly with more than two synthetic nucleic acid molecules, such as three, four, five, six, seven, eight, nine, or 10 synthetic nucleic acid molecules using the teachings herein.

In some examples, the system includes a nucleic acid molecule that suppresses expression of un-assembled/un-recombined fragments. In such an example, if the two or more portions of a full-length coding sequence (e.g., 114 of 110, 164 of 150 of FIGS. 6A and 6G, respectively), did not recombine, the nucleic acid molecule would suppress expression of each portion of a full-length coding sequence that was not recombined into a full-length nucleic acid editing protein. For example, such a suppressive nucleic acid molecule can destabilize the RNA once outside the nucleus, prevent translation, stimulate translation from a shifted start codon, contain microRNA target sites, or contain protein degron or destabilization domains that when translated suppress the protein activity or flag it for degradation.

In one example, destabilization of the un-recombined RNA molecule is achieved by including a self-cleaving RNA sequence (e.g., Hammerhead ribozyme or HDV ribozyme) into the synthetic intron, for example at any position within intronic sequence 130 of FIG. 6A, 6F, or 6G. In one example, cleaving the RNA molecule leads to a loss of the RNA stabilizing poly A tail, which can suppress expression of an un-recombined protein from open reading frame 114 of FIG. 6A, 6F, or 6G. In one example, a self-cleaving RNA sequence is included at any position within s intronic sequence 170 of FIG. 6A, 6F, or 6G to cleave off the 5′ terminal CAP which in one example can lead to reduced expression of an open reading frame that includes parts or the whole of coding sequence 164 of FIG. 6A, 6F, or 6G. In one example self-cleaving RNA sequences are substituted with an RNA cleaving enzyme target site, such as a Csy4 target site.

In some examples, a suppressive nucleic acid molecule includes a start codon (ATG) or a Kozak enhanced start codon (GCCGCCACCATG (SEQ ID NO: 154) or GCCACCATG or ACCATG) at any position within intronic sequence 170 of FIG. 6A, 6F, or 6G that directs translation of an open reading frame that is shifted −1, −2, +1, or +2 nucleotides relative to the open reading frame sequence 164 of FIG. 6A, 6F, or 6G. In one example, un-assembled fragment expression is reduced or suppressed by using this decoy start codon strategy to direct translation away from the to be suppressed open reading frame of sequence 164 of FIG. 6A, 6F, or 6G.

In some examples, a suppressive nucleic acid molecule includes one or more micro RNA target sites at any position within intronic sequence 130 of FIG. 6A, 6F, or 6G, and/or at any position within intronic sequence 170 of FIG. 6A or 6F. If a particular molecule (e.g., 110 or 150 in FIG. 6A, 6F, or 6G) is exported from the nucleus, it becomes subject to micro RNA/small hairpin RNA dependent degradation which can suppress unintended un-joined fragment expression by degrading/suppressing un-joined RNA that was exported from the nucleus. In one example, such a micro RNA target sequence can be complementary to a micro RNA known to be expressed in the cell, or tissue, or animal into which the molecules 110 and 150 of FIG. 6A, 6F, or 6G are introduced. In one example, this micro RNA target sequence is complementary to a sequence that is introduced into the cell, or tissue, or animal. In one example, such a microRNA can be expressed from an RNA-polymerase III dependent promoter in the form of a small hairpin RNA. In one example, such a microRNA can be expressed from an RNA polymerase II dependent promoter and embedded in a micro RNA processing loop (e.g., mir30 scaffold).

In some examples, destabilization of the un-recombined protein product from an open reading frame (e.g., 114 in FIGS. 6A-6H) can be achieved by depleting stop codon occurrence in intronic sequence 130 of FIG. 6A, 6F, or 6G and an additional inclusion of an RNA sequence coding for an in frame protein signal that can flag a protein for degradation (e.g., a degron sequence) that is placed at any position within intronic sequence 130 of FIG. 6A, 6F, or 6G and which is in frame with the open reading frame that is extended out from sequence 114 of FIG. 6A, 6F, or 6G. In one example a degron sequence can be that of a PEST sequence, or that of the CLI degron sequence. Degron sequences used can employ proteasome-dependent, proteasome-independent, ubiquitin-dependent, or ubiquitin-independent pathways. In one example, un-recombined protein destabilization is enhanced by inclusion of several of the same or different degron sequences.

In some examples, destabilization of the un-recombined protein product from open reading frame sequence 164 in any of FIGS. 6A-6H is achieved by introduction of a start codon (ATG) followed by a degron sequence at any position within intronic sequence 170 in F any of FIGS. 6A-6H which is in frame with an open reading frame within sequence 164 in any of FIGS. 6A-6H. In this example, the degron sequence will be N-terminally joined to the un-recombined protein fragment that will be suppressed by being flagged for degradation.

### IV. Exemplary Nucleic Acid Editors

As shown in FIGS. 6A-6H, the disclosed RNA recombination methods, which use two (or more) nucleic acid molecules 110, 200, 150, can be used to express a nucleic acid editing protein. As shown in FIGS. 6G and 6H, in some examples, nucleic acid molecules 110, 200, 150 further include one or more gRNA coding sequences (e.g., 140, 141, 231, 323, 171, 172), whose expression can be driven by a promoter (e.g., 142, 143, 322, 234, 173, 174). However, in some examples, nucleic acid molecules 110, 200, 150 do not include an gRNA coding sequence, and instead, such gRNA coding sequences (and promoters) can be provided on one or more other synthetic DNA molecules (such as one or more vectors). In some examples, a system, DNA composition, or RNA composition provided herein includes two or more gRNA molecules or coding sequences, which in some examples can be part of one or more cassettes. In some examples, a system, DNA composition, or RNA composition provided herein includes two or more gRNA molecules or coding sequences, such as 2, 3, 4, 5, 6, 7, 8, 9 or 10 gRNA molecules or coding sequences, wherein each gRNA can be the same (e.g., multiple copies of the same gRNA), or different (e.g., a gRNA for each particular target), or combinations thereof (e.g., multiple gRNA for multiple targets).

Proteins having the ability to edit a nucleic acid sequence can be used to edit a DNA or RNA sequence, such as a gene sequence. In some examples, such a protein is used to (a) delete or remove one or more nucleotides or ribonucleotides from a target nucleic acid molecule (b) insert one or more nucleotides or ribonucleotides from a target nucleic acid molecule, (c) substitute one or more nucleotides or ribonucleotides in a target nucleic acid molecule, or combinations of (a), (b) and (c). In particular examples, a nucleic acid editing protein is a nuclease, such as an RNA guided nuclease. In one example, a nucleic acid editing protein is a Cas nuclease. Exemplary Cas nucleases include those that can edit a DNA molecule (such as a genomic sequence), such as Cas9 and dCas9, as well as those that can edit RNA, such as Cas13d and dCas13d. Other exemplary nucleic acid editing proteins include TALENS, and zinc finger nucleases. In some examples, the nucleic acid editing protein is a fusion protein, which includes another peptide or protein. In one example, the nucleic acid editor coding sequence (e.g., 114, 216, 164) is codon optimized for mammalian or human cells.

In one example, the nucleic acid editor includes a Cas nuclease for editing DNA, such as a Cas9 from Streptococcus pyogenes (SpCas9), Cas9 from Staphylococcus aureus (SaCas9), Cas9 from Streptococcus thermophilus (StCas9), Cas9 from Neisseria meningitidis (NmCas9), Cas9 from Francisella novicida (FnCas9), Cas9 from Campylobacter jejuni (CjCas9), CasX, CasY, Cas12a (Cpf1), Cas12b (C2c1), Cas14a. In one example, the nucleic acid editor is a Cas nuclease for editing DNA, such as a Cas9 nickase, high-fidelity Cas9 (SpCas9-HF-1), eSPCas9, HypaCas9, FokI-fused dCas9, xCas9, SpRY, or SpG. In one example, the nucleic acid editor is a catalytically dead Cas9 (dCas9). In one example, the nucleic acid editor is a fusion protein that includes any of these Cas nucleases for editing DNA, and at least one other peptide or protein (e.g., a cytosine base editor, an adenine base editor, or both). In some examples, the Cas nuclease is at the N- or C-terminus of the fusion protein.

In one example, the nucleic acid editor is a Cas nuclease for editing RNA, such as Cas13a, Cas13b, Cas13c and Cas13d. In one example, the nucleic acid editor is a catalytically dead Cas13a (dCas13a), Cas13b (dCas13b, such as dPspCas13b), Cas13c (dCas13c) or Cas13d (dCas13d). In one example, the nucleic acid editor is a fusion protein that includes any of these Cas nucleases for editing DNA, and at least one other peptide or protein (e.g., a cytosine base editor, an adenine base editor, or both). In some examples, the Cas nuclease is at the N- or C-terminus of the fusion protein.

In one example, the nucleic acid editor is a fusion protein that includes an inactive/dead Cas enzyme (such as dCas9 or dCas13) and epigenetic modifier, such as p300, LSD1, MQ1, and TET1, for programmable epigenome-engineering. In some examples, the Cas nuclease is at the N- or C-terminus of the fusion protein.

Thus, in some examples, the nucleic acid editor is divided into two (or more) portions, wherein each portion is encoded by a different nucleic acid molecule 110, 200, 150. For example, if the protein is divided into two sections (e.g., in half or roughly in half) an N-terminal portion of the nucleic acid editor 114 can be encoded by nucleic acid molecule 110, while the C-terminal portion of the nucleic acid editor 164 can be encoded by nucleic acid molecule 150. In an example where the system or composition is DNA, one or more promoters 112, 152 can be included to drive expression of the coding sequences 114, 164. If the system or composition is RNA, the one or more promoters 112, 202, 152, the gRNA coding sequences 140, 141, 231, 232, 171, 172 and their corresponding promoters 142, 143, 233, 234, 173, 174, and the TRs 176, 177, 235, 236, 176, 179 are absent. In some examples, the nucleic acid editor protein (which may be part of a fusion protein) is at least 500 amino acids, at least 600 aa, at least 700 aa, at least 800 aa, at least 900 aa, at least 1000 aa, at least 1100 aa, at least 1200 aa, or at least 1300aa.

In some examples, the DNA or RNA to be edited by the disclosed methods includes one or more mutations, such as one or more nucleotide substitutions, deletions or additions, which can be edited to include the native/non-mutated sequence. In one example, such methods are used to edit a DNA or RNA sequence to modulate (i.e., upregulate or downregulate) expression of a target DNA or RNA in a cell, such as a human cell, such as one in a subject. In one example, multiple targets are edited simultaneously or contemporaneously (such as at least two different targets, for example by using multiple gRNAs, such as 2, 3, 4, 5, 6, 7, 8, 9 or 10 different targets). In some examples, multiple targets are in the same gene. In other examples, multiple targets are in different genes. Examples of targets are provided in Tables 1-4.

A. CRISPR/Cas9 or CRISPR/dCas9 In some examples, the N- and C-terminal coding portion 114, 164 of molecules 110, 150 (and if three molecules are used, middle coding portion 216 of 200) encodes a Cas nuclease to edits a DNA sequence, such as a gene sequence. In some examples N- and C-terminal coding portion 114, 164 of molecules 110, 150 (and if three molecules are used, middle coding portion 216 of 200) encode a Cas9 protein (e.g., SEQ ID NO: 208) or a dCas9 protein (e.g., SEQ ID NO: 210). In such examples, a DNA composition including 110, 150 can further include one or more gRNA coding sequences. The gRNA is designed based on the particular Cas9 or dCas9 protein encoded by 114, 164 (or 114, 216, 164), and is used to direct a Cas9 or a dCas9 protein to a target nucleic acid sequence. The gRNA in examples for DNA editing include a crispr RNA (crRNA) having a portion complementary to the target DNA (e.g., at least 10 nt, at least 12 nt, at least 13 nt, at least 14, nt, at least 15 nt, at least 16 nt, at least 17 nt, at least 18 nt, at least nt, or at least 25 nt, such as 14-30 nt, such as 17-20 nt) and a tracrRNA (binding scaffold for the Cas nuclease). Changing the targeting sequence within the crRNA portion of the gRNA allows targeting of any DNA of interest. In some examples, the gRNA is a dead guide RNA (dgRNA). In some examples, the use of a Cas9 or dCas9 protein requires the presence of a PAM sequence at the target locus/sequence in the DNA to be edited. One or more gRNAs can be encoded on molecule 110, molecule 150, or both, for example expressed from a promoter. In some examples, one or more gRNAs can be encoded by one or more additional DNA molecules, such as a different vectors.

In some examples, a native or wild-type Cas9 sequence is used (e.g., SEQ ID NOS: 207 and 208). In one example, a mutated Cas9 sequence is used (e.g., SEQ ID NOS: 209 and 210). In one example a mutated “nickase” version of Cas9 is used (D10A mutant of the Cas9 nuclease enzyme), which generates a single-strand DNA break, instead of a ds break. In one example a catalytically inactive Cas9 (dCas9) is used to knockdown gene expression by interfering with transcription of a target nucleic acid molecule. The dCas9 can be fused to an additional repressor peptide. A catalytically inactive Cas9 (dCas9) fused to an activator peptide can activate or increase gene expression (for example to treat a genetic disorder in which upregulation of a target gene is desired).

In one example, a Cas9 protein encoded by 114, 164 or 114, 216, 164, or a Cas9 portion of a fusion protein encoded by 114, 164 or 114, 216, 164, has at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 208, and retains DNA endonuclease activity. In one example, a Cas9 coding sequence of 114, 164 or 114, 216, 164, or the Cas9 coding sequence portion of a fusion protein encoded by 114, 164 or 114, 216, 164, has at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 207, and encodes a protein having DNA endonuclease activity.

In one example, a dCas9 protein encoded by 114, 164 or 114, 216, 164, or a dCas9 portion of a fusion protein encoded by 114, 164 or 114, 216, 164, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 210, and has reduced or no DNA endonuclease activity but can bind to dsDNA, and in some examples includes one or more D10A, E762A, D839A, H840A, N854A, N863A, and D986A substitutions. In one example, a dCas9 coding sequence of 114, 164 or 114, 216, 164, or a dCas9 coding sequence portion of a fusion protein encoded by 114, 164 or 114, 216, 164, has at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 209, and encodes a protein with has reduced or no DNA endonuclease activity but can bind to dsDNA, and in some examples encodes a protein having one or more D10A, E762A, D839A, H840A, N854A, N863A, and D986A substitutions.

In some examples, the Cas9 or dCas9 encoded by 114, 164 or 114, 216, 164, is a fusion protein, which further includes another protein or peptide, for example at the N- or C-terminus of Cas9 or dCas9, or anywhere within Cas9 or dCas9.

A Cas9 or dCas9 encoded by a composition provided herein can further include one or more nuclear localization signals (NLSs). Thus, in some examples an NLS-Cas9 or NLS-dDas9 fusion protein is encoded. Exemplary NLS sequences include SPKKKRKVEAS (SEQ ID NO: 218; e.g., encoded by AGCCCCAAGAAgAAGAGaAAGGTGGAGGCCAGC, SEQ ID NO: 219) and GPKKKRKVAAA (SV40 large T antigen NLS, SEQ ID NO: 220; e.g., encoded by ggacctaagaaaaagaggaaggtggcggccgct, SEQ ID NO: 221). In some examples, the NLS is at the N-terminus of the Cas9 or dCas9 protein. In some examples, the NLS is at the C-terminus of the Cas9 or dCas9 protein. In some examples, an NLS-Cas9 or NLS-dDas9 fusion protein includes two or more NLSs, for example at the N-terminus and at the C-terminus of the Cas9/dCas9 protein. In some examples, the NLS is within of the Cas9/dCas9 protein.

In one example, the Cas9 or dCas9 fusion protein includes a transcriptional activation domain, such as VP64, P65, MyoD1, HSF1, RTA, CBP, SET7/9, or any combination thereof. Thus, in some examples the 114, 164 or 114, 216, 164, encodes a fusion protein that includes Cas9 or dCas9 and one or more of VP64, P65, MyoD1, HSF1, RTA, CBP, and SET7/9. In one example, the fusion protein is dCas9-VP64 (which can include one or more copies of VP64, such as 1, 2, 3, 4, or 5 VP64 proteins). In one example, the fusion protein is dCas9-VP64-P65-Rta (VPR). In one example, the fusion protein is dCas9-CBP. CBP is a histone acetyltransferase domain. The presence of a transcriptional activation domain can be used to activate transcription of a target gene. In some examples, Cas9 or dCas9 does not further include a transcriptional activation domain, such as VP64, P65, MyoD1, HSF1, RTA, CBP, SET7/9, or any combination thereof.

In some examples, the Cas9 or dCas9 encoded by 114, 164 or 114, 216, 164, is a fusion protein, which further includes a base editor (BE), such as a cytosine base editor (CBE) or adenine base editor (ABE). CBEs mediate a C to T change (or a G to A change on the opposite strand). ABEs make an A to G change (or a T to C change on the opposite strand).

In some examples, the Cas9 or dCas9 encoded by 114, 164 or 114, 216, 164, is a fusion protein, which further includes a cytosine base editor (CBE), which corrects T•A to C•G point mutations in DNA. An exemplary CBE is cytidine deaminase (such as one from sea lamprey [AID], CDA1, or APOBEC3G). These fusions convert cytosine to uracil without cutting DNA. Uracil is then subsequently converted to thymine through DNA replication or repair. Fusing an inhibitor of uracil DNA glycosylase (UGI) to dCas9 prevents base excision repair which changes the U back to a C mutation. In one example, a Cas nickase-cytidine deaminase fusion protein (BE3) is used, which nicks the unmodified DNA strand so that it appears “newly synthesized” to the cell. Thus, the cell repairs the DNA using the U-containing strand as a template, copying the base edit. In one example, the fusion protein is high fidelity Cas9 variant HF-Cas9 fused to cytidine deaminase (HF-BE3). In one example, a Cas nickase-cytidine deaminase fusion protein (Target-AID) is used. In one example, fusion protein HF-Cas9-BE3 is used. In one example, fusion protein BE4, BE4max, AncBE4 or AncBE4max is used. In one example, the fusion protein includes a cytidine deaminase fused to an impaired form of Cas9 (D10A nickase) tethered to one (BE3) or two (BE4i monomers of uracil glycosylase inhibitor (UGI), which s enables the conversion of C•G base pairs to T•A base pair in human genomic DNA, through the formation of a uracil intermediate. In one example, the fusion protein includes BE4 with either RrA3F, AmAPOBEC1, SsAPOBEC3B, or PpAPOBEC1. In one example, the fusion protein includes BE4 with either RrA3F [wt, F130L], AmAPOBEC1, SsAPOBEC3B [wt, R54Q], or PpAPOBEC1 [wt, H122A, R33A].

In one example, the Cas9 or dCas9 encoded by 114, 164 or 114, 216, 164, is a fusion protein, which further includes an adenine base editor (ABE), which can convert adenine to inosine, resulting in the conversion of A•T to G•C in genomic DNA. Exemplary ABEs include ABE 6.3, 7.8, 7.9 and 7.10, as well as ABEmax, ABE8s (e.g., ABE8e(TadA-8e V106W), see Richter et al., Nature biotech. 38:883-91, 2020)).

In one example, the Cas9 or dCas9 encoded by 114, 164 or 114, 216, 164, is a fusion protein, which further includes an ABE and a CBE, such as SPACE (using miniABEmax-V82G and Target-AID to Cas9), and a fusion of both cytidine and adenosine deaminases with a Cas9 nickase.

In one example, the Cas9 or dCas9 encoded by 114, 164 or 114, 216, 164, is a fusion protein, which further includes a reverse transcriptase. In one example, the dCas9 is dCas9 H840A nickase. In some such examples, the gRNA used is a prime editing gRNA (pegRNA) which is longer than a typical gRNA. The pegRNA includes an extended gRNA containing a primer binding site (PBS) and a reverse transcriptase (RT) template sequence.

In one example, the Cas9 or dCas9 encoded by 114, 164 or 114, 216, 164, is a fusion protein, which further includes bacteriophage protein Gam (for example to the N-terminus of Cas9 or dCas9, which can further include a cytidine deaminase (e.g., AID, CDA1, or APOBEC3G)).

In some examples, instead of using a Cas9 nuclease, a CRISPR RNA-guided Fok1 nuclease is used (e.g., see Tsai et al., Nature Biotechnol. 32:569-76, 2014). Thus, in some examples 114, 164 or 114, 216, 164, include a Fok1 coding sequence. Dimeric RNA-guided FokI nucleases (RFNs) can recognize extended sequences and edit endogenous genes in human cells. RFN cleavage activity depends on the binding of two guide RNAs (gRNAs) to DNA with a defined spacing and orientation.

### B. CRISPR/Cas13d or CRISPR/dCas13d

In some examples, the N- and C-terminal coding portion 114, 164 of molecules 110, 150 (and if three molecules are used, middle coding portion 216 of 200) encodes a Cas13d protein (e.g., SEQ ID NOS: 212, 214, 222) to edit an RNA sequence. In some examples N- and C-terminal coding portion 114, 164 of molecules 110, 150 (and if three molecules are used, middle coding portion 216 of 200) encode a dead Cas13d (dCas13d) protein (e.g., SEQ ID NO: 216) to edit an RNA sequence. In such examples, a DNA composition including 110, 150 can further include one or more gRNA coding sequences. The gRNA is designed based on the particular Cas13d or dCas13 protein encoded by 114, 164 (or 114, 216, 164), and is used to direct a Cas13d or dCas13 protein to a target nucleic acid sequence. The gRNA in examples for DNA editing include a (1) crispr RNA (crRNA) containing a direct repeat (DR) region having secondary structure which facilitates interaction between the Cas13d or dCas13d and the gRNA (e.g., at least 10 nt, at least 12 nt, at least 13 nt, at least 14, nt, at least 15 nt, at least 16 nt, at least 17 nt, at least 18 nt, at least 20 nt, at least 25 nt, at least 30 nt, or at least 36 nt, such as 20-40 nt, such as 30-36 nt) and (2) a spacer having a portion complementary to the target RNA (e.g., at least 10 nt, at least 12 nt, at least 13 nt, at least 14, nt, at least 15 nt, at least 16 nt, at least 17 nt, at least 18 nt, at least 20 nt, at least 25 nt, at least 30 nt, or at least 32 nt, such as 20-40 nt, 30-32 nt, or 25-35 nt). In some examples, the gRNA (or DNA encoding such) includes a constant direct repeat DR at its 5′ end and a variable spacer at its 3′ end. In some examples, unprocessed gRNA is 36 nt of DR followed by 30-32 nt of spacer sequence. Following its expression, the gRNA is processed (truncated/modified) by Cas13d or dCas13 or other RNases into its shorter “mature” form. Changing the targeting sequence within the spacer portion of the gRNA allows targeting of any RNA of interest. In some examples, the use of a Cas13d or dCas13d protein negates the requirement for the presence of a PAM sequence at the target locus/sequence in the RNA to be edited. One or more gRNAs can be encoded on molecule 110, molecule 150, or both, for example expressed from a promoter. In some examples, one or more gRNAs can be encoded by one or more additional DNA molecules, such as a different vectors. The DR sequence depends on the Cas13d protein. For example, if Cas13d shown in SEQ ID NO 212 is used, the DR sequence of the gRNA can be 5′CAAGUAAACCCCUACCAACUGGUCGGGGUUUGAAAC 3′ (SEQ ID NO: 223; underline indicates sequence of the predicted mature form, a spacer having about 10-40 nt or 14-30 nt complementary to the target RNA can be added at the 3′ end of the DR). For example, if Cas13d shown in SEQ ID NO 214 is used, the DR sequence of the gRNA can be 5′CUACUACACUGGUGCGAAUUUGCACUAGUCUAAAAC 3′ (SEQ ID NO: 224; underline indicates sequence of the predicted mature form, a spacer having about 10-40 nt or 14-30 nt complementary to the target RNA can be added at the 3′ end of the DR).

In some examples, a native or wild-type or native Cas13d sequence is used (e.g., SEQ ID NOS: 207 and 208). In one example, a mutated Cas9 sequence is used (e.g., SEQ ID NOS: 212, 214 and 222). In one example a mutated version of Cas13d is used, such as a dead Cas 13d (a catalytically inactive for of Cas13 having one or both mutated HEPN domain(s) and thus cannot cut RNA, but can process gRNA, see SEQ ID NO: 216).

In one example, a Cas13d protein encoded by 114, 164 or 114, 216, 164, or a dCas13d portion of a fusion protein encoded by 114, 164 or 114, 216, 164, has at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 212, 214, or 222, and retains RNA endonuclease activity. In one example, a Cas13d coding sequence of 114, 164 or 114, 216, 164, or the Cas13d coding sequence portion of a fusion protein encoded by 114, 164 or 114, 216, 164, has at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 211 or 213, and encodes a protein having RNA endonuclease activity.

In one example, a dCas13d protein encoded by 114, 164 or 114, 216, 164, or a dCas13d portion of a fusion protein encoded by 114, 164 or 114, 216, 164, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 216, and is catalytically inactive but can process to gRNA, and in some examples includes mutations in one or both HEPN domains. In one example, a dCas13d coding sequence of 114, 164 or 114, 216, 164, or a dCas13d coding sequence portion of a fusion protein encoded by 114, 164 or 114, 216, 164, has at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 215, and encodes a protein that is catalytically inactive but can process to gRNA, and in some examples encodes mutations in one or both HEPN domains.

In some examples, the Cas13d or dCas13d encoded by 114, 164 or 114, 216, 164, is a fusion protein, which further includes another protein or peptide, for example at the N- or C-terminus of as13d or dCas13d, or anywhere within as13d or dCas13d.

A Cas13d or dCas13d encoded by a composition provided herein can further include one or more nuclear localization signals (NLSs). Thus, in some examples an NLS-Cas13d or NLS-dDas13d fusion protein is encoded. Exemplary NLS sequences include SPKKKRKVEAS (SEQ ID NO: 218; e.g., encoded by AGCCCCAAGAAgAAGAGaAAGGTGGAGGCCAGC, SEQ ID NO: 219) and GPKKKRKVAAA (SV40 large T antigen NLS, SEQ ID NO: 220; e.g., encoded by ggacctaagaaaaagaggaaggtggcggccgct, SEQ ID NO: 221). In some examples, the NLS is at the N-terminus of the as13d or dCas13d protein. In some examples, the NLS is at the C-terminus of the as13d or dCas13d protein. In some examples, an NLS-Cas13d or NLS-dDas13d fusion protein includes two or more NLSs, for example at the N-terminus and at the C-terminus of the as13d or dCas13d protein. In some examples, the NLS is within of the Cas13d or dCas13d protein.

In some examples, the Cas13d or dCas13d encoded by 114, 164 or 114, 216, 164, is a fusion protein, which further includes a base editor (BE), such as an RNA base editor, such as ADAR (adenosine deaminase acting on RNA). This protein converts adenine to inosine.

### C. Zinc-Finger Nucleases (ZFNs) and Transcription Activator-Like Effector Nucleases (TALENs)

Zinc-finger nucleases (ZFNs) and transcription activator-like effector nucleases (TALENs) are chimeric nucleases composed of programmable, sequence-specific DNA-binding modules linked to a nonspecific DNA cleavage domain (such as Fok1 nuclease). ZFNs and TALENs enable a broad range of genetic modifications by inducing DNA double-strand breaks that stimulate error-prone nonhomologous end joining or homology-directed repair at specific genomic locations (for review see Gaj et al., Trends Biotechnol. 31:397-405, 2013). Zinc-finger proteins and TALEs can be fused to enzymatic domains, such as site-specific nucleases, recombinases and transposases, which catalyze DNA integration, excision, and inversion. For applications that require targeted gene addition, recombinase and transposase activity is marked by the insertion of donor DNA into the genome, thereby enabling off-target effects to be monitored directly.

In some examples, the N- and C-terminal coding portion 114, 164 of molecules 110, 150 (and if three molecules are used, middle coding portion 216 of 200) encodes at least one (such as 1 or 2) ZFN protein or ZFN fusion proteins to edit a DNA sequence, such as a gene sequence. In such an example, gRNA sequences are not included (e.g., 140, 141, 142, 143, 171, 172, 173, and 174 are omitted).

In zinc finger nuclease based engineering, a ZFN is used to cut genomic DNA at a desired location. Two ZFNs are used, with each containing two functional domains. The first is a DNA-binding domain comprised of a chain of at least two (such as 2, 3, 4, 5 or 6) zinc finger modules, each recognizing a unique hexamer (6 bp) sequence of DNA. Two-finger modules are stitched together to form a Zinc Finger Protein, each with specificity of ≥24 bp. The second is a DNA-cleaving domain that includes a Fok1 endonuclease (such as the catalytic domain). When the DNA-binding and DNA-cleaving domains are fused together, a highly-specific pair of ‘genomic scissors’ are created. This permits editing of a genome, for example to downregulate or upregulate a gene. For example, expression of two ZFNs in a cell using the disclosed systems and compositions results in the ZFN pair recognizing and heterodimerizing around the target site. The ZFN pair makes a double strand break and then dissociates from the target DNA. If a corresponding repair template is co-transfected with the ZFN pair, this will result in repair of the target gene (e.g., repair a deletion, insertion, substitution, or other genetic alteration) by homologous recombination. Thus, the repair template can be designed to repair a mutated gene or upregulate gene expression or activity. If no corresponding repair template is co-transfected with the ZFN pair, this will result in disruption of the target gene (e.g., downregulation due to mutations introduced by nonhomologus end joining).

Several approaches can be used to design specific zinc finger nucleases for the target nucleic acid(s), such as a gene listed in Tables 1-4 (or other gene associated with the disorders provided herein). The most widespread involves combining zinc-finger units with known specificities (modular assembly). Various selection techniques, using bacteria, yeast or mammal cells have been developed to identify the combinations that offer the best specificity and the best cell tolerance. A zinc finger DNA binding domain is a protein domain that binds DNA in a sequence-specific manner through one or more zinc fingers, which are regions of amino acid sequence within the binding domain whose structure is stabilized through coordination of a zinc ion. Zinc finger binding domains, for example the recognition helix of a zinc finger, can be “engineered” to bind to a predetermined nucleotide sequence (e.g., engineer it to bind to CXCR4 or other target). Rational criteria for design of zinc finger binding domains include application of substitution rules and computerized algorithms for processing information in a database storing information of existing ZFN pair designs and binding data, see for example U.S. Pat. Nos. 5,789,538; 5,925,523; 6,007,988; 6,013,453; 6,140,081; 6,200,759; 6,453,242; and 6,534,261; and PCT Publication Nos. WO 95/19431; WO 96/06166; WO 98/53057; WO 98/53058; WO 98/53059; WO 98/53060; WO 98/54311; WO 00/27878; WO 01/60970; WO 01/88197; WO 02/016536; WO 02/099084 and WO 03/016496.

In some examples, two different sets (pairs) of ZFNs are used, for example such that one set is specific for one target nucleic acid molecule, while the other set is specific for a second target nucleic acid molecule. One skilled in the art will appreciate that more than two different sets of ZFNs can be used (e.g., at least 2, at least 3, at least 4, or at least 5 different sets, such as 2, 3, 4, 5, 6, 7, 8, 9, or 10 different sets). In one example, multiple combinations of 110, 150 (or 110, 150, 200) of molecules are used, each wherein combination expresses one ZFN.

In some examples N- and C-terminal coding portion 114, 164 of molecules 110, 150 (and if three molecules are used, middle coding portion 216 of 200) encode a TALEN or TALEN fusion protein to edit a DNA sequence, such as a gene sequence. Methods for designing TALENs (e.g., see Bogdanove and Voytas, Science. 333(6051):1843-6, 2011; Cermak et al., Nucleic Acids Res. 39:e82, 2011; Sander et al., Nat Biotechnol. 29(8):697-8, 2011) and performing TALEN-mediated gene targeting (e.g., see Hockenmeyer et al., Nat Biotechnol 29: 731-734) can be applied to the present disclosure.

In the TALEN method, the TALE DNA binding domains, which can be designed to bind any desired DNA sequence (such as a gene in Tables 1-4), come from TAL effectors, DNA-binding proteins excreted by certain bacteria that infect plants (Xanthomonas). These are combined with a DNA cleavage domain. The DNA binding domain contains a repeated highly conserved 33-35 amino acid sequence with the exception of the 12th and 13th amino acids. These two locations are highly variable (Repeat Variable Diresidue, RVD) and have specific nucleotide recognition. This relationship between amino acid sequence and DNA recognition has allowed for the engineering of specific DNA binding domains by selecting a combination of repeat segments containing the appropriate RVDs. The non-specific DNA cleavage domain, for example from the end of a FokI endonuclease, can be used to construct hybrid nucleases. The FokI domain functions as a dimer, so that using two constructs with unique DNA binding domains for sites in the target genome with proper orientation and spacing allows excellent specificity. Both the number of amino acid residues between the TALE DNA binding domain and the FokI cleavage domain and the number of bases between the two individual TALEN binding sites can be varied.

TALENs are used in a similar way to design zinc finger nucleases. TALE specificity is determined by two hypervariable amino acids known as the repeat-variable diresidues (RVDs). Like zinc-fingers, modular TALE repeats are linked together to recognize contiguous DNA sequences. However in contrast to zinc finger proteins, there is no re-engineering of the linkage between repeats necessary to construct long arrays of TALEs with the ability to address single sites in the genome.

In some examples, two different sets (pairs) of TALENs are used, for example such that one set is specific for one target nucleic acid molecule, while the other set is specific for a second target nucleic acid molecule. One skilled in the art will appreciate that more than two different sets of ZFNs can be used (e.g., at least 2, at least 3, at least 4, or at least 5 different sets, such as 2, 3, 4, 5, 6, 7, 8, 9, or 10 different sets). In one example, multiple combinations of 110, 150 (or 110, 150, 200) of molecules are used, wherein each combination expresses one TALEN.

The compositions and methods of the present disclosure can be used to achieve the expression of a full-length nucleic acid editor protein in vivo, for example to treat genomic point mutations. For example, a first RNA molecule comprises a first portion of the nucleic acid editor protein coding sequence that is appended to a first synthetic RNA dimerization and recombination domain (that is an intron and binding domain). This molecule is expressed from a first vector/plasmid. A second RNA molecule comprises a second portion of the nucleic protein coding sequence appended to the complementary second RNA dimerization and recombination domain and is expressed from a second vector/plasmid. One or both of these DNA vectors/plasmids (first and second DNA molecules) can contain a gRNA/guideRNA/gRNA expression cassette composed of an RNA polymerase III promoter and the gRNA/guideRNA/gRNA sequence as illustrated for example in FIG. 6G. For example, a composition of the present disclosure may include 1-8 gRNA/guideRNA/gRNA expression cassettes. The number of gRNA/guideRNA/gRNA expression cassettes in a composition of the present disclosure may be 1 to 2, 1 to 3, 1 to 4, 1 to 5, 1 to 6, 1 to 7, 1 to 8, 2 to 3, 2 to 4, 2 to 5, 2 to 6, 2 to 7, 2 to 8, 3 to 4, 3 to 5, 3 to 6, 3 to 7, 3 to 8, 4 to 5, 4 to 6, 4 to 7, 4 to 8, 5 to 6, 5 to 7, 5 to 8, 6 to 7, 6 to 8, or 7 to 8. The number of gRNA/guideRNA/gRNA expression cassettes in a composition of the present disclosure may be 1, 2, 3, 4, 5, 6, 7, or 8. The number of gRNA/guideRNA/gRNA expression cassettes in a composition of the present disclosure may be at least 1, 2, 3, 4, 5, 6, or 7. The number of gRNA/guideRNA/gRNA expression cassettes in a composition of the present disclosure may be at most 2, 3, 4, 5, 6, 7, or 8. When the two RNA molecules are expressed in the same target cell, the two portions of the nucleic acid editor protein coding transcript are recombined to form the full-length nucleic acid editor protein transcript which is then translated into protein.

A first DNA molecule may comprise the following sequences, from 5′ to 3′: An AAV Inverted Terminal Repeat; a gRNA sequence (reverse orientation); a promoter (reverse orientation); a promoter; a 5′ untranslated region; an N-terminal portion of a nucleic acid editor coding sequence; a synthetic intron sequence comprising and/or overlapping with a first Dimerization Domain; a poly adenylation signal sequence; a promoter; a gRNA sequence; and an AAV Inverted Terminal Repeat. A second DNA molecule may comprise the following sequences, from 5′ to 3′: An AAV Inverted Terminal Repeat; a gRNA sequence (reverse orientation); a promoter (reverse orientation), a promoter, a synthetic intron sequence comprising and/or overlapping with a second Dimerization Domain; a C-terminal portion of the nucleic acid editor coding sequence; a poly adenylation signal sequence; a promoter; a gRNA sequence; and an AAV Inverted Terminal Repeat.

A first DNA molecule may comprise the following sequences, from 5′ to 3′: An AAV Inverted Terminal Repeat; a gRNA sequence (reverse orientation); an RNA polymerase III promoter (reverse orientation); a promoter; a 5′ untranslated region; an N-terminal portion of a nucleic acid editor coding sequence; a synthetic intron sequence comprising and/or overlapping with a first Dimerization Domain; a poly adenylation signal sequence; an RNA polymerase III promoter; a gRNA sequence; and an AAV Inverted Terminal Repeat. A second DNA molecule may comprise the following sequences, from 5′ to 3′: An AAV Inverted Terminal Repeat; a gRNA sequence (reverse orientation); an RNA polymerase III promoter (reverse orientation), a promoter, a synthetic intron sequence comprising and/or overlapping with a second Dimerization Domain; a C-terminal portion of the nucleic acid editor coding sequence; a poly adenylation signal sequence; an RNA polymerase III promoter; a gRNA sequence; and an AAV Inverted Terminal Repeat.

A first DNA molecule may comprise the following sequences, from 5′ to 3′: An AAV2 Inverted Terminal Repeat; a gRNA sequence (reverse orientation); an RNA polymerase III promoter (reverse orientation); a promoter; a 5′ untranslated region; an N-terminal portion of a nucleic acid editor coding sequence; a synthetic intron sequence comprising and/or overlapping with a first Dimerization Domain; a poly adenylation signal sequence; an RNA polymerase III promoter; a gRNA sequence; and an AAV2 Inverted Terminal Repeat. A second DNA molecule may comprise the following sequences, from 5′ to 3′: An AAV2 Inverted Terminal Repeat; a gRNA sequence (reverse orientation); an RNA polymerase III promoter (reverse orientation), a promoter, a synthetic intron sequence comprising and/or overlapping with a second Dimerization Domain; a C-terminal portion of the nucleic acid editor coding sequence; a poly adenylation signal sequence; an RNA polymerase III promoter; a gRNA sequence; and an AAV2 Inverted Terminal Repeat.

A first DNA molecule may comprise the following sequences, from 5′ to 3′: An AAV2 Inverted Terminal Repeat; a gRNA sequence (reverse orientation); an RNA polymerase III promoter (reverse orientation); a CMV promoter; a 5′untranslated region; an N-terminal portion of a nucleic acid editor coding sequence; a synthetic intron sequence comprising and/or overlapping with a first Dimerization Domain; a poly adenylation signal sequence; an H1 RNA polymerase III promoter, a gRNA sequence; and an AAV2 Inverted Terminal Repeat. A second DNA molecule may comprise the following sequences, from 5′ to 3′: An AAV2 Inverted Terminal Repeat; a gRNA sequence (reverse orientation); a human U6 RNA polymerase III promoter (reverse orientation), a CMV promoter; a synthetic intron sequence comprising and/or overlapping with a second Dimerization Domain; a C-terminal portion of the nucleic acid editor coding sequence; a poly adenylation signal sequence; an H1 RNA polymerase III promoter; a gRNA sequence; and an AAV2 Inverted Terminal Repeat.

In some embodiments, the first DNA molecule comprises a sequence that encodes a first RNA molecule encoding an N-terminal portion of dCas9-VPR, an N-terminal portion of Prime Editor, an N-terminal portion of AncBE4, or an N-terminal portion of ABE8e, and the second DNA molecule comprises a sequence that encodes a first RNA molecule encoding an C-terminal portion of dCas9-VPR, a C-terminal portion of Prime Editor, an C-terminal portion of AncBE4, or a C-terminal portion of ABE8e, respectively.

In some embodiments, the sequence of the first DNA molecule that encodes the first RNA molecule encoding the N-terminal portion of the dCas9-VPR protein comprises SEQ ID NO: 159. In some embodiments, the sequence has a percent sequence identity to SEQ ID NO: 159 of about 80% to about 100%. In some embodiments, the sequence has a % sequence identity to SEQ ID NO: 159 of about 80 to about 100. In some embodiments, the sequence has a % sequence identity to SEQ ID NO: 159 of about 80 to about 85, about 80 to about 90, about 80 to about 95, about 80 to about 96, about 80 to about 97, about 80 to about 98, about 80 to about 99, about 80 to about 100, about 85 to about 90, about 85 to about 95, about 85 to about 96, about 85 to about 97, about 85 to about 98, about 85 to about 99, about 85 to about 100, about 90 to about 95, about 90 to about 96, about 90 to about 97, about 90 to about 98, about 90 to about 99, about 90 to about 100, about 95 to about 96, about 95 to about 97, about 95 to about 98, about 95 to about 99, about 95 to about 100, about 96 to about 97, about 96 to about 98, about 96 to about 99, about 96 to about 100, about 97 to about 98, about 97 to about 99, about 97 to about 100, about 98 to about 99, about 98 to about 100, or about 99 to about 100. In some embodiments, the sequence has a % sequence identity to SEQ ID NO: 159 of about 80, about 85, about 90, about 95, about 96, about 97, about 98, about 99, or about 100.

In some embodiments, the sequence has a % sequence identity to SEQ ID NO: 159 of at least about 80, about 85, about 90, about 95, about 96, about 97, about 98, or about 99. In some embodiments, the sequence has a % sequence identity to SEQ ID NO: 159 of at most about 85, about 90, about 95, about 96, about 97, about 98, about 99, or about 100.

In some embodiments, the sequence of the second DNA molecule that encodes the first RNA molecule encoding the C-terminal portion of the dCas9-VPR protein comprises SEQ ID NO: 160. In some embodiments, the sequence has a percent sequence identity to SEQ ID NO: 160 of about 80% to about 100%. In some embodiments, the sequence has a % sequence identity to SEQ ID NO: 160 of about 80 to about 100. In some embodiments, the sequence has a % sequence identity to SEQ ID NO: 160 of about 80 to about 85, about 80 to about 90, about 80 to about 95, about 80 to about 96, about 80 to about 97, about 80 to about 98, about 80 to about 99, about 80 to about 100, about 85 to about 90, about 85 to about 95, about 85 to about 96, about 85 to about 97, about 85 to about 98, about 85 to about 99, about 85 to about 100, about 90 to about 95, about 90 to about 96, about 90 to about 97, about 90 to about 98, about 90 to about 99, about 90 to about 100, about 95 to about 96, about 95 to about 97, about 95 to about 98, about 95 to about 99, about 95 to about 100, about 96 to about 97, about 96 to about 98, about 96 to about 99, about 96 to about 100, about 97 to about 98, about 97 to about 99, about 97 to about 100, about 98 to about 99, about 98 to about 100, or about 99 to about 100. In some embodiments, the sequence has a % sequence identity to SEQ ID NO: 160 of about 80, about 85, about 90, about 95, about 96, about 97, about 98, about 99, or about 100. In some embodiments, the sequence has a % sequence identity to SEQ ID NO: 160 of at least about 80, about 85, about 90, about 95, about 96, about 97, about 98, or about 99. In some embodiments, the sequence has a % sequence identity to SEQ ID NO: 160 of at most about 85, about 90, about 95, about 96, about 97, about 98, about 99, or about 100.

In some embodiments, the sequence that encodes the RNA encoding the N-terminal portion of the Prime Editor protein comprises SEQ ID NO: 161. In some embodiments, the sequence has a percent sequence identity to SEQ ID NO: 161 of about 80% to about 100%. In some embodiments, the sequence has a % sequence identity to SEQ ID NO: 161 of about 80 to about 100. In some embodiments, the sequence has a % sequence identity to SEQ ID NO: 161 of about 80 to about 85, about 80 to about 90, about 80 to about 0.99. 95, about 80 to about %, about 80 to about 97, about 80 to about 98, about 80 to about 99, about 80 to about 100, about 85 to about 90, about 85 to about 95, about 85 to about %, about 85 to about 97, about 85 to about 98, about 85 to about 99, about 85 to about 100, about 90 to about 95, about 90 to about %, about 90 to about 97, about 90 to about 98, about 90 to about 99, about 90 to about 100, about 95 to about %, about 95 to about 97, about 95 to about 98, about 95 to about 99, about 95 to about 100, about % to about 97, about % to about 98, about % to about 99, about % to about 100, about 97 to about 98, about 97 to about 99, about 97 to about 100, about 98 to about 99, about 98 to about 100, or about 99 to about 100. In some embodiments, the sequence has a % sequence identity to SEQ ID NO: 161 of about 80, about 85, about 90, about 95, about 96, about 97, about 98, about 99, or about 100. In some embodiments, the sequence has a % sequence identity to SEQ ID NO: 161 of at least about 80, about 85, about 90, about 95, about 96, about 97, about 98, or about 99. In some embodiments, the sequence has a % sequence identity to SEQ ID NO: 161 of at most about 85, about 90, about 95, about 96, about 97, about 98, about 99, or about 100.

In some embodiments, the sequence that encodes the RNA encoding the C-terminal portion of the Prime Editor protein comprises SEQ ID NO: 162. In some embodiments, the sequence has a percent sequence identity to SEQ ID NO: 162 of about 80% to about 100%. In some embodiments, the sequence has a % sequence identity to SEQ ID NO: 162 of about 80 to about 100. In some embodiments, the sequence has a % sequence identity to SEQ ID NO: 162 of about 80 to about 85, about 80 to about 90, about 80 to about 95, about 80 to about 96, about 80 to about 97, about 80 to about 98, about 80 to about 99, about 80 to about 100, about 85 to about 90, about 85 to about 95, about 85 to about 96, about 85 to about 97, about 85 to about 98, about 85 to about 99, about 85 to about 100, about 90 to about 95, about 90 to about 96, about 90 to about 97, about 90 to about 98, about 90 to about 99, about 90 to about 100, about 95 to about 96, about 95 to about 97, about 95 to about 98, about 95 to about 99, about 95 to about 100, about 96 to about 97, about 96 to about 98, about 96 to about 99, about 96 to about 100, about 97 to about 98, about 97 to about 99, about 97 to about 100, about 98 to about 99, about 98 to about 100, or about 99 to about 100. In some embodiments, the sequence has a % sequence identity to SEQ ID NO: 162 of about 80, about 85, about 90, about 95, about 96, about 97, about 98, about 99, or about 100. In some embodiments, the sequence has a % sequence identity to SEQ ID NO: 162 of at least about 80, about 85, about 90, about 95, about 96, about 97, about 98, or about 99. In some embodiments, the sequence has a % sequence identity to SEQ ID NO: 162 of at most about 85, about 90, about 95, about 96, about 97, about 98, about 99, or about 100.

In some embodiments, the sequence that encodes the RNA encoding the N-terminal portion of the AncBE4 protein comprises SEQ ID NO: 163. In some embodiments, the sequence has a percent sequence identity to SEQ ID NO: 163 of about 80% to about 100%. In some embodiments, the sequence has a % sequence identity to SEQ ID NO: 163 of about 80 to about 100. In some embodiments, the sequence has a % sequence identity to SEQ ID NO: 163 of about 80 to about 85, about 80 to about 90, about 80 to about 95, about 80 to about 96, about 80 to about 97, about 80 to about 98, about 80 to about 99, about 80 to about 100, about 85 to about 90, about 85 to about 95, about 85 to about 96, about 85 to about 97, about 85 to about 98, about 85 to about 99, about 85 to about 100, about 90 to about 95, about 90 to about 96, about 90 to about 97, about 90 to about 98, about 90 to about 99, about 90 to about 100, about 95 to about %, about 95 to about 97, about 95 to about 98, about 95 to about 99, about 95 to about 100, about % to about 97, about % to about 98, about % to about 99, about % to about 100, about 97 to about 98, about 97 to about 99, about 97 to about 100, about 98 to about 99, about 98 to about 100, or about 99 to about 100. In some embodiments, the sequence has a % sequence identity to SEQ ID NO: 163 of about 80, about 85, about 90, about 95, about 96, about 97, about 98, about 99, or about 100. In some embodiments, the sequence has a % sequence identity to SEQ ID NO: 163 of at least about 80, about 85, about 90, about 95, about %, about 97, about 98, or about 99. In some embodiments, the sequence has a % sequence identity to SEQ ID NO: 163 of at most about 85, about 90, about 95, about %, about 97, about 98, about 99, or about 100.

In some embodiments, the sequence that encodes the RNA encoding the C-terminal portion of the AncBE4 protein comprises SEQ ID NO: 164. In some embodiments, the sequence has a percent sequence identity to SEQ ID NO: 164 of about 80% to about 100%. In some embodiments, the sequence has a % sequence identity to SEQ ID NO: 164 of about 80 to about 100. In some embodiments, the sequence has a % sequence identity to SEQ ID NO: 164 of about 80 to about 85, about 80 to about 90, about 80 to about 95, about 80 to about %, about 80 to about 97, about 80 to about 98, about 80 to about 99, about 80 to about 100, about 85 to about 90, about 85 to about 95, about 85 to about %, about 85 to about 97, about 85 to about 98, about 85 to about 99, about 85 to about 100, about 90 to about 95, about 90 to about 96, about 90 to about 97, about 90 to about 98, about 90 to about 99, about 90 to about 100, about 95 to about %, about 95 to about 97, about 95 to about 98, about 95 to about 99, about 95 to about 100, about % to about 97, about % to about 98, about % to about 99, about % to about 100, about 97 to about 98, about 97 to about 99, about 97 to about 100, about 98 to about 99, about 98 to about 100, or about 99 to about 100. In some embodiments, the sequence has a % sequence identity to SEQ ID NO: 164 of about 80, about 85, about 90, about 95, about 96, about 97, about 98, about 99, or about 100. In some embodiments, the sequence has a % sequence identity to SEQ ID NO: 164 of at least about 80, about 85, about 90, about 95, about %, about 97, about 98, or about 99. In some embodiments, the sequence has a % sequence identity to SEQ ID NO: 164 of at most about 85, about 90, about 95, about 96, about 97, about 98, about 99, or about 100.

In some embodiments, the sequence that encodes the RNA encoding the N-terminal portion of the ABE8e protein comprises SEQ ID NO: 165. In some embodiments, the sequence has a percent sequence identity to SEQ ID NO: 165 of about 80% to about 100%. In some embodiments, the sequence has a % sequence identity to SEQ ID NO: 165 of about 80 to about 100. In some embodiments, the sequence has a % sequence identity to SEQ ID NO: 165 of about 80 to about 85, about 80 to about 90, about 80 to about 95, about 80 to about %, about 80 to about 97, about 80 to about 98, about 80 to about 99, about 80 to about 100, about 85 to about 90, about 85 to about 95, about 85 to about %, about 85 to about 97, about 85 to about 98, about 85 to about 99, about 85 to about 100, about 90 to about 95, about 90 to about %, about 90 to about 97, about 90 to about 98, about 90 to about 99, about 90 to about 100, about 95 to about %, about 95 to about 97, about 95 to about 98, about 95 to about 99, about 95 to about 100, about % to about 97, about % to about 98, about % to about 99, about % to about 100, about 97 to about 98, about 97 to about 99, about 97 to about 100, about 98 to about 99, about 98 to about 100, or about 99 to about 100. In some embodiments, the sequence has a % sequence identity to SEQ ID NO: 165 of about 80, about 85, about 90, about 95, about 96, about 97, about 98, about 99, or about 100. In some embodiments, the sequence has a % sequence identity to SEQ ID NO: 165 of at least about 80, about 85, about 90, about 95, about 96, about 97, about 98, or about 99. In some embodiments, the sequence has a % sequence identity to SEQ ID NO: 165 of at most about 85, about 90, about 95, about 96, about 97, about 98, about 99, or about 100.

In some embodiments, the sequence that encodes the RNA encoding the C-terminal portion of the ABE8e protein comprises SEQ ID NO: 166. In some embodiments, the sequence has a percent sequence identity to SEQ ID NO: 166 of about 80% to about 100%. In some embodiments, the sequence has a % sequence identity to SEQ ID NO: 166 of about 80 to about 100. In some embodiments, the sequence has a % sequence identity to SEQ ID NO: 166 of about 80 to about 85, about 80 to about 90, about 80 to about 95, about 80 to about 96, about 80 to about 97, about 80 to about 98, about 80 to about 99, about 80 to about 100, about 85 to about 90, about 85 to about 95, about 85 to about 96, about 85 to about 97, about 85 to about 98, about 85 to about 99, about 85 to about 100, about 90 to about 95, about 90 to about 96, about 90 to about 97, about 90 to about 98, about 90 to about 99, about 90 to about 100, about 95 to about 96, about 95 to about 97, about 95 to about 98, about 95 to about 99, about 95 to about 100, about 96 to about 97, about 96 to about 98, about 96 to about 99, about 96 to about 100, about 97 to about 98, about 97 to about 99, about 97 to about 100, about 98 to about 99, about 98 to about 100, or about 99 to about 100. In some embodiments, the sequence has a % sequence identity to SEQ ID NO: 166 of about 80, about 85, about 90, about 95, about 96, about 97, about 98, about 99, or about 100. In some embodiments, the sequence has a % sequence identity to SEQ ID NO: 166 of at least about 80, about 85, about 90, about 95, about 96, about 97, about 98, or about 99. In some embodiments, the sequence has a % sequence identity to SEQ ID NO: 166 of at most about 85, about 90, about 95, about 96, about 97, about 98, about 99, or about 100.

The sequence of the first DNA molecule, that encodes a first RNA molecule encoding an N-terminal portion of the ABESe protein, and additionally comprises at least one gRNA/guideRNA/gRNA expression cassette, may comprise SEQ ID NO: 225. In some embodiments, the sequence has a % sequence identity to SEQ ID NO: 225 of about 80 to about 100. In some embodiments, the sequence has a % sequence identity to SEQ ID NO: 225 of about 80 to about 85, about 80 to about 90, about 80 to about 95, about 80 to about 96, about 80 to about 97, about 80 to about 98, about 80 to about 99, about 80 to about 100, about 85 to about 90, about 85 to about 95, about 85 to about 96, about 85 to about 97, about 85 to about 98, about 85 to about 99, about 85 to about 100, about 90 to about 95, about 90 to about 96, about 90 to about 97, about 90 to about 98, about 90 to about 99, about 90 to about 100, about 95 to about 96, about 95 to about 97, about 95 to about 98, about 95 to about 99, about 95 to about 100, about 96 to about 97, about 96 to about 98, about 96 to about 99, about 96 to about 100, about 97 to about 98, about 97 to about 99, about 97 to about 100, about 98 to about 99, about 98 to about 100, or about 99 to about 100. In some embodiments, the sequence has a % sequence identity to SEQ ID NO: 225 of about 80, about 85, about 90, about 95, about 96, about 97, about 98, about 99, or about 100. In some embodiments, the sequence has a % sequence identity to SEQ ID NO: 225 of at least about 80, about 85, about 90, about 95, about %, about 97, about 98, or about 99. In some embodiments, the sequence has a % sequence identity to SEQ ID NO: 225 of at most about 85, about 90, about 95, about %, about 97, about 98, about 99, or about 100.

The sequence of the second DNA molecule, that encodes a second RNA molecule encoding an N-terminal portion of the ABE8e protein, and additionally comprises at least one gRNA/guideRNA/gRNA expression cassette, may comprise SEQ ID NO: 226. In some embodiments, the sequence has a % sequence identity to SEQ ID NO: 226 of about 80 to about 100. In some embodiments, the sequence has a % sequence identity to SEQ ID NO: 226 of about 80 to about 85, about 80 to about 90, about 80 to about 95, about 80 to about %, about 80 to about 97, about 80 to about 98, about 80 to about 99, about 80 to about 100, about 85 to about 90, about 85 to about 95, about 85 to about %, about 85 to about 97, about 85 to about 98, about 85 to about 99, about 85 to about 100, about 90 to about 95, about 90 to about 96, about 90 to about 97, about 90 to about 98, about 90 to about 99, about 90 to about 100, about 95 to about %, about 95 to about 97, about 95 to about 98, about 95 to about 99, about 95 to about 100, about % to about 97, about % to about 98, about 96 to about 99, about % to about 100, about 97 to about 98, about 97 to about 99, about 97 to about 100, about 98 to about 99, about 98 to about 100, or about 99 to about 100. In some embodiments, the sequence has a % sequence identity to SEQ ID NO: 226 of about 80, about 85, about 90, about 95, about %, about 97, about 98, about 99, or about 100. In some embodiments, the sequence has a % sequence identity to SEQ ID NO: 226 of at least about 80, about 85, about 90, about 95, about %, about 97, about 98, or about 99. In some embodiments, the sequence has a % sequence identity to SEQ ID NO: 226 of at most about 85, about 90, about 95, about 96, about 97, about 98, about 99, or about 100.

### IV. Compositions and Kits

Compositions and kits are provided that include two or more of the synthetic nucleic acid molecules provided herein, wherein the two or more (such as 2, 3, 4, 5, 6, 7, 8, 9 or 10) synthetic nucleic acid molecule encode a full-length protein when recombined. In some examples, the two or more of the synthetic nucleic acid molecules provided herein are DNA. In some examples, the two or more of the synthetic nucleic acid molecules provided herein are RNA, and do not include promoter sequences. In one example, the composition or kit includes two of the synthetic nucleic acid molecules provided herein, wherein each of the two synthetic nucleic acid molecules encodes a different portion of a nucleic acid editing protein (i.e., N-terminal and C-terminal, wherein the whole coding sequence is generated when recombination between the two molecules occurs. In one example, the composition or kit includes three of the synthetic nucleic acid molecules provided herein, wherein each of the three synthetic nucleic acid molecules encodes a different portion of a nucleic acid editing protein (i.e., N-terminal, middle, and C-terminal, wherein the whole coding sequence is generated when recombination between the three molecules occurs), such as Cas9, a dCas9, a Cas13d, or dCas13d (or a fusion protein of any one of these). In one example, the composition or kit includes four or more of the synthetic nucleic acid molecules provided herein, wherein each of the four of more synthetic nucleic acid molecules encodes a different portion of a nucleic acid editing protein (i.e., N-terminal, first middle, second middle (and optionally additional middle), and C-terminal, wherein the whole coding sequence is generated when recombination between the four or more synthetic nucleic acid molecules occurs). In one example, the composition or kit includes two or more sets of two or more of the synthetic nucleic acid molecules provided herein, wherein each set of synthetic nucleic acid molecules encodes a different nucleic acid editing protein. In some examples, the compositions and kits further include a nucleic acid molecule containing one or more gRNAs (such as a cassette containing multiple gRNAs), or a nucleic acid molecule encoding one or more gRNA coding sequences (for example encoding a cassette containing multiple gRNAs), which can be operably linked to a promoter.

In one example, each synthetic nucleic acid molecule in the composition or kit is part of a vector, such as AAV or other gene therapy vector. In one example, the composition or kit includes a cell, such as a bacterial cell or eukaryotic cell, that includes two or more disclosed synthetic nucleic acid molecules, wherein the synthetic nucleic acid molecules encode a full-length nucleic acid editing protein protein when recombined.

Such compositions can include a pharmaceutically acceptable carrier (e.g., saline, water, glycerol, DMSO, or PBS). In some examples, the composition is a liquid, lyophilized powder, or cryopreserved.

In some examples, the kit includes a delivery system (e.g., liposome, a particle, an exosome, or a microvesicle) to direct cell type specific uptake/enhance endosomal escape/enable blood-brain barrier crossing etc. In some examples, the kits further include cell culture or growth media, such as media appropriate for growing bacterial, plant, insect, or mammalian cells. In some examples, such parts of a kit are in separate containers. Exemplary containers include plastic or glass vials or tubes.

In some examples, each of two or more the synthetic nucleic acid molecules provided herein are in separate containers. In some examples, each of two or more sets of two or more of the synthetic nucleic acid molecules provided herein are in separate containers.

### V. Methods of Treatment

The disclosed methods and systems can be used to express any nucleic acid editing protein of interest, for example when a protein is too large to be expressed by a therapeutic virus (e.g., AAV) or when a complete gene sequence (e.g., endogenous promoter+coding sequence) is too large to be expressed by a therapeutic virus (e.g., AAV). In such cases, the coding sequence of the nucleic acid editing protein protein may be divided into two or more portions using the disclosed systems, and recombined in the correct order, allowing for the protein to be expressed when and where desired. The compositions and systems can further include one or more gRNAs (or nucleic acid molecules encoding one or more gRNAs operably linked to a promoter), which target a nucleic acid molecule to be edited (for example to treat a disease listed in any of Tables 1-4).

The subject to be treated can be any mammal, such as one with a monogenetic disorder, such as one listed in Tables 1-4. In one example, the subject has cancer. Thus, humans, cats, pigs, rats, mice, cows, goats, and dogs, can be treated with the disclosed methods. In some examples, the subject is a human infant less than 6 months of age. In some examples, the subject is a human infant less than 1 year of age. In some examples, the subject is a human juvenile. In some examples, the subject is a human adult at least 18 years of age. In some examples, the subject is female. In some examples, the subject is male.

The two or more synthetic nucleic acid molecules provided herein used to treat a subject can be matched to the subject treated. Thus, for example, if the subject to be treated is a dog, a dog coding sequence for the nucleic acid editing protein can be used and the intronic sequence can be optimized for expression in dog cells, and if the subject to be treated is a human, a human coding sequence for the nucleic acid editing protein can be used and the intronic sequence can be optimized for expression in human cells.

The two or more synthetic nucleic acid molecules provided herein can be administered as part of a vector, such as an adeno-associated vector (AAV), for example AAV serotype rh.10. In some examples, vectors (e.g., AAV) including one of the two or more synthetic nucleic acid molecules provided herein are administered systemically, such as intravenously. Thus, if a coding sequence is divided between two synthetic nucleic acid molecules provided herein, two AAV's are administered, each AAV including one of the two synthetic nucleic acid molecules provided herein.

A therapeutically effective amount of two or more synthetic nucleic acid molecules provided herein is administered, for example in AAVs. In some examples, the two or more synthetic nucleic acid molecules provided herein when part of a viral vector (e.g., AAV) is administered at a dose of at least 1×10¹⁰ genome copies (gc), at least 1×10¹¹ gc, at least 2×10¹¹ gc, at least 1×10¹² gc, at least 2×10¹¹ gc, at least 1×10¹³ gc, at least 2×10¹³ gc per subject, or at least 1×10¹⁴ gc per subject, such as 2×10¹⁰ gc per subject, 2×10¹¹ gc per subject, 2×10¹² gc per subject, 2×10¹³ gc per subject, or 2×10¹⁴ gc per subject. In some examples, the two or more synthetic nucleic acid molecules provided herein when part of a viral vector (e.g., AAV) is administered at a dose of at least 1×10¹⁰ gc/kg, at least 5×10¹⁰ gc/kg, at least 1×10¹¹ gc/kg, at least 5×10¹¹ gc/kg, at least 1×10¹² gc/kg, at least 5×10¹² gc/kg, at least 1×10¹³ gc/kg, or at least 4×10¹³ gc/kg, such as 4×10¹⁰ gc/kg, 4×10¹¹ gc/kg, 4×10¹² gc/kg, or 4×10¹³ gc/kg.

In some examples, the two or more synthetic nucleic acid molecules provided herein as part of a viral vector (e.g., AAV) is administered at a dose of about 1×10¹⁰ genome copies (gc) to about 1×10¹⁴ per subject, or per treatment area of a subject. In some embodiments, the dose administered per subject, or per treatment area of a subject is about 1×10¹⁰ gc to about 1×10¹⁴ gc. In some embodiments, the dose administered per subject, or per treatment area of a subject is about 1×10¹⁰ gc to about 1×10¹¹ gc, about 1×10¹⁰ gc to about 1×10¹² gc, about 1×10¹⁰ gc to about 1×10¹³ gc, about 1×10¹⁰ to about 1×10¹⁴ gc, about 1×10¹¹ gc to about 1×10¹² gc, about 1×10¹¹ gc to about 1×10¹³ gc, about 1×10¹¹ gc to about 1×10¹⁴ gc, about 1×10¹² gc to about 1×10¹³gc, about 1×10¹² gc to about 1×10¹¹ gc, or about 13 gc to about 1×10¹⁰ gc. In some embodiments, the dose administered per subject, or per treatment area of a subject is about 1×10¹⁰ gc, about 1×10¹¹ gc, about 1×10¹²gc, about 1×10¹³ gc, or about 1×10¹⁴ gc. In some embodiments, the dose administered per subject, or per treatment area of a subject is at least about 1×10¹⁰ gc, about 1×10¹¹ gc, about 1×10¹² gc, or about 1×10¹³gc. In some embodiments, the dose administered per subject, or per treatment area of a subject is at most about 1×10¹¹ gc, about 1×10¹² gc, about 1×10¹³ gc, or about 1×10¹⁴ gc. In some embodiments, administration of a therapeutically effective amount of a system of the disclosure restores expression of a target nucleic acid and/or production of protein from the target nucleic acid when compared with a control (e.g., untreated). In some embodiments, administration of a therapeutically effective amount of a system of the disclosure, comprising a dose of about 1×10¹⁰ gc to about 1×10¹⁴ gc, including about 1×10¹⁰ gc to about 1×10¹¹, including about 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 1×10¹¹ gc per subject or per treatment area of the subject, restores expression of a target nucleic acid and/or production of protein from the target nucleic acid when compared with a control (e.g., untreated). In some embodiments, the restored expression compared with control expression is about 20% to 100%, or greater. In some embodiments, the restored expression compared with control expression is about 20% to about 100%. In some embodiments, the restored expression compared with control expression is about 20% to about 30%, about 20% to about 40%, about 20% to about 50%, about 20% to about 60%, about 20% to about 70%, about 20% to about 75%, about 20% to about 80%, about 20% to about 85%, about 20% to about 90%, about 20% to about 95%, about 20% to about 100%, about 30% to about 40%, about 30% to about 50%, about 30% to about 60%, about 30% to about 70%, about 30% to about 75%, about 30% to about 80%, about 30% to about 85%, about 30% to about 90%, about 30% to about 95%, about 30% to about 100%, about 40% to about 50%, about 40% to about 60%, about 40% to about 70%, about 40% to about 75%, about 40% to about 80%, about 40% to about 85%, about 40% to about 90%, about 40% to about 95%, about 40% to about 100%, about 50% to about 60%, about 50% to about 70%, about 50% to about 75%, about 50% to about 80%, about 50% to about 85%, about 50% to about 90%, about 50% to about 95%, about 50% to about 100%, about 60% to about 70%, about 60% to about 75%, about 60% to about 80%, about 60% to about 85%, about 60% to about 90%, about 60% to about 95%, about 60% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the restored expression compared with control expression is about 20%, about 30%, about 40%, about 50%, about 60%, about 70%, about 75%, about 80%, about 85%, about 90%, about 95%, or about 100%. In some embodiments, the restored expression compared with control expression is at least about 20%, about 30%, about 40%, about 50%, about 60%, about 70%, about 75%, about 80%, about 85%, about 90%, or about 95%. In some embodiments, the restored expression compared with control expression is at most about 30%, about 40%, about 50%, about 60%, about 70%, about 75%, about 80%, about 85%, about 90%, about 95%, or about 100%. In some embodiments, the restored protein is dystrophin and the treatment area is a muscle or muscle group. In some embodiments, the muscle or muscle group is the tibialis anterior (TA) hindlimb muscle of a test animal, e.g., a mouse. In some embodiments, administration is by injection (e.g., subcutaneous, intramuscular, intradermal, intraperitoneal, intrathecal, intratumoral, intraosseous, or intravenous).

In some embodiments, administration of a therapeutically effective amount of a system of the disclosure reduces expression of a target nucleic acid and/or production of protein from the target nucleic acid when compared with a control (e.g., untreated). In some embodiments, administration of a therapeutically effective amount of a system of the disclosure, comprising a dose of about 1×10¹⁰ gc to about 1×10¹⁴ gc, including about 1×10¹⁰ gc to about 1×10¹¹, including about 1×10¹⁰, 2×10¹⁰, 3×10¹⁰, 4×10¹⁰, 5×10¹⁰, 6×10¹⁰, 7×10¹⁰, 8×10¹⁰, 9 ×10¹⁰, or 1×10¹¹ gc per subject or per treatment area of the subject, reduces expression of a target nucleic acid and/or production of protein from the target nucleic acid when compared with a control (e.g., untreated). In some embodiments, the reduced expression compared with control expression is about 20% to 100%. In some embodiments, the reduced expression compared with control expression is about 20% to about 100%. In some embodiments, the reduced expression compared with control expression is about 20% to about 30%, about 20% to about 40%, about 20% to about 50%, about 20% to about 60%, about 20% to about 70%, about 20% to about 75%, about 20% to about 80%, about 20% to about 85%, about 20% to about 90%, about 20% to about 95%, about 20% to about 100%, about 30% to about 40%, about 30% to about 50%, about 30% to about 60%, about 30% to about 70%, about 30% to about 75%, about 30% to about 80%, about 30% to about 85%, about 30% to about 90%, about 30% to about 95%, about 30% to about 100%, about 40% to about 50%, about 40% to about 60%, about 40% to about 70%, about 40% to about 75%, about 40% to about 80%, about 40% to about 85%, about 40% to about 90%, about 40% to about 95%, about 40% to about 100%, about 50% to about 60%, about 50% to about 70%, about 50% to about 75%, about 50% to about 80%, about 50% to about 85%, about 50% to about 90%, about 50% to about 95%, about 50% to about 100%, about 60% to about 70%, about 60% to about 75%, about 60% to about 80%, about 60% to about 85%, about 60% to about 90%, about 60% to about 95%, about 60% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In some embodiments, the reduced expression compared with control expression is about 20%, about 30%, about 40%, about 50%, about 60%, about 70%, about 75%, about 80%, about 85%, about 90%, about 95%, or about 100%. In some embodiments, the reduced expression compared with control expression is at least about 20%, about 30%, about 40%, about 50%, about 60%, about 70%, about 75%, about 80%, about 85%, about 90%, or about 95%. In some embodiments, the reduced expression compared with control expression is at most about 30%, about 40%, about 50%, about 60%, about 70%, about 75%, about 80%, about 85%, about 90%, about 95%, or about 100%.

If adverse symptoms develop, such as AAV-capsid specific T cells in the blood, corticosteroids can be administered (e.g., see Nathwani et al., N Engl J Med. 365(25):2357-65, 2011).

Diseases that can be treated with the disclosed methods include any genetic disease of the blood (e.g. sickle cell disease, primary immunodeficiency diseases), HIV (such as HIV-1), and hematologic malignancies or cancers. Examples of primary imnmunodeficiency diseases and their corresponding mutations include those listed in Al-Herz et al. (Frontiers in Immunology, volume 5, article 162, Apr. 22, 2014, herein incorporated by reference in its entirety). Hematologic malignancies or cancers are those tumors that affect blood, bone marrow, and lymph nodes. Examples include leukemia (e.g., acute lymphoblastic leukemia, acute myelogenous leukemia, chronic lymphocytic leukemia, chronic myelogenous leukemia, acute monocytic leukemia), lymphoma (e.g., Hodgkin's lymphoma and non-Hodgkin's lymphoma), and myeloma. In some examples, the disease is a monogenetic disease. Tables 1 provides a list of exemplary disorders and genes that can be targeted by the disclosed systems and methods. Additional examples are provided here: rarediseases.info.nigov/diseasesdisases-by-category/lcongenital-and-genetic-diseases (list herein incorporated by reference). Any genetic disease caused by a lack of protein (e.g., recessive mutation) or an insufficiency of protein can benefit from the disclosed systems and methods. In cases where the coding region of the gene is relatively small, the disclosed systems and methods are useful to add regulatory sequences, such as tissue specific promoters or specific non-coding RNA segments, to direct gene expression to the appropriate cell types at the appropriate levels.

TABLE 4
Exemplary disorders and corresponding mutations

| Disease | Gene | Mutation |
|---|---|---|
| Blood cell disorder | | |
| sickle cell anemia | β-globin chain of | SNP (A to T) that gives rise to |
| | hemoglobin | point mutation (Glu −> Val at 6^{th} |
| | | aa) |
| hemophilia | any of clotting factors I | |
| | through XIII | |
| hemophilia A | clotting factor VIII | large deletions, insertions, |
| | | inversions, and point mutations |
| hemophilia B | clotting factor IX | |
| Alpha-Thalassemia | HBA1 or HBA2 | Mutation or a deletion in |
| | | chromosome 16 p |
| Beta-Thalassemia | HBB | Mutations in chromosome 11 |
| Delta-Thalassemia | HBD | mutation |
| von Willebrand Disease | von Willebrand factor | mutations or deletion |
| pernicious anemia | MTHFR | |
| Fanconi anemia | FANCA, FANCC, | FANCA: c.3788_3790del |
| | FANCD2, FANCG, | (p.Phe1263del); |
| | FANCJ | c.1115_1118delTTGG |
| | | (p.Val372fs); Exon 12-17del; |
| | | Exon 12-31del; c.295C > T |
| | | (p.Gln99X) |
| | | FANCC: c.711 + 4A > T (originally |
| | | reported as IVS4 + 4A > T); |
| | | c.67delG (originally reported as |
| | | 322delG) |
| | | FANCD2: c.1948-16T > G |
| | | FANCG; c.313G > T |
| | | (p.Glu105X); c.1077-2A > G; |
| | | c.1480 + 1G > C; c.307 + 1G > C; |
| | | c.1794_1803del (p.Trp599fs); |
| | | c.637_643del (p.Tyr213fs) |
| | | FANCJ: c.2392C > T |
| | | (p.Arg798X) |
| Thrombocytopenic purpura | ADAMTS13 | Missense and nonsense mutations |
| thrombophilia | Factor V Leiden | Mutation in the F5 gene at |
| | Prothrombin | position 1691 |
| | | Prothrombin G20210A |
| Primary Immunodeficiency | | |
| Diseases | | |
| T-B+ SCID | IL-2RG, JAK3, defect in | |
| | gamma chain of receptors | |
| | for IL-2, -4, -7, -9, -15 and - | |
| | 21 | |
| T-B- SCID | RAG1, RAG2 | |
| WHIM syndrome | CXCR4 | heterozygous mutations (e.g., in |
| | | the carboxy-terminus); carboxy- |
| | | terminus truncation (e.g., 10-19 |
| | | residues) |
| Other Primary immune | | |
| deficiency (PID) syndromes | | |
| IL-7 receptor severe combined | IL7 receptor | |
| immune deficiency (SCID) | | |
| Adenosine deaminase deficiency | ADA | |
| (ADA) SCID | | |
| Purine nucleoside phosphorylase | PNP | |
| (PNP) deficiency | | |
| Wiskott-Aldrich syndrome | WAS | More than 300 mutations |
| (WAS) | | identified |
| Chronic granulomatous disease | CYBA, CYBB, NCF1, | |
| (CGD) | NCF2, or NCF4 | |
| Leukocyte adhesion deficiency | Beta-2 integrin | |
| (LAD) | | |
| HIV | C-C chemokine receptor | Deletion of 32 bp in CCR5 |
| | type 5 (CCR5), | |
| | MSRB1 | |
| | HIV long terminal repeats | |
| | CSCR4 | |
| | P17 | |
| | PSIP1 | |
| Duchenne muscular dystrophy | CCR5 | |
| | DMD | |
| Glycogen storage disease type IA | G6Pase | |
| Retinal Dystrophy | CEP290 | C2991 + 1655A > G |
| | ABCA4 | 5196 + 1216C > A; |
| | | 5196 + 1056A > G; |
| | | 5196 + 1159G > A; |
| | | 5196 + 1137G > A; |
| | | 938-619A > G; 4539 + 2064C > T |
| X-linked immunodeficiency with | MAGT1 | |
| magnesium defect, Epstein-Barr | | |
| virus infection, and neoplasia | | |
| (XMEN) | | |
| MonoGenetic Disorders | | |
| Metachromatic leukodystrophy | arylsulfatase A (ARSA) | |
| (MLD) | | |
| Adrenoleukodystrophy (ALD) | ABCD1 | |
| Mucopolysaccaridoses (MPS) | IDS | |
| disorders | IDUA | |
| Hunter syndrome | IDUA | |
| Hurler syndrome | SGSH, NAGLU, | |
| Scheie syndrome | HGSNAT, GNS | |
| Sanfilippo syndrome A, B, C, and | GALNS | |
| D | GLB1 | |
| Morquio syndrome A Morquio | ARSB | |
| syndrome B | GUSB | |
| Maroteaux-Lamy syndrome | HYAL1 | |
| Sly syndrome | | |
| Natowicz syndrome | | |
| Alpha manosidosis | MAN2B1 | |
| Nieman Pick disease types A, B, | SMPD1, NPC1, NPC2 | |
| and C | | |
| Cystic fibrosis | cystic fibrosis | ΔF508 |
| | transmembrane | |
| | conductance regulator | |
| | (CFTR) | |
| Polycystic kidney disease | PKD-1, PDK-2, PDK-3 | |
| Tay Sachs Disease | HEXA | 1278insTATC |
| Gaucher disease | GBA | |
| Huntington's disease | HTT | CAG repeat |
| Neurofibromatosis types 1 and 2 | NF-1 and NF2 | CGA −> UGA −> Arg1306Term in |
| | | NF1 |
| Familial hypercholesterolemia | APOB, LDLR, LDLRAP1, | |
| | and PCSK9 | |
| Cancers | | |
| Chronic myeloid leukemia (CML) | BCR-ABL | fusion |
| | ASXL1 | |
| Acute myeloid leukemia (AML) | Chromosome 11q23 or | translocation |
| | t(9; 11) | |
| Osteosarcoma | RUNX2 | |
| Colorectal cancer | EPHA1 | |
| Gastric cancer, melanoma | PD-1 | |
| Prostate cancer | Androgen receptor | |
| Cervical cancer | E6, E7 | |
| Glioblastoma | CD | |
| Neurological disorders | | |
| Alzheimer's disease | NGF | |
| Metahchromatic leukodystrophy | ARSA | |
| Multiple sclerosis | MBP | |
| Wiskott-Aldrich syndrome | WASP | |
| X-linked adrenoleukodystrophy | ABCD1 | |
| AACD deficiency | AADC | |
| Batten disease | CLN2 | |
| Canavan disease | ASPA | |
| Giant axonal neuropathy | GAN | |
| Leber's hereditary optic | MT-ND4 | |
| neuropathy | | |
| MPS IIIA | SGSH, SUMF1 | |
| Parkinson's disease | GAD, NTRN, TH, AADC, | |
| | CH1, GDNF, AADC | |
| Pompe disease | GAA | |
| Spinal muscular atrophy type 1 | SMN | |

Using the disclosed methods and systems can be used to treat any of the disorders listed in Table 1, or other known genetic disorder. The disclosed methods can also be used to treat other disorders, such as a cancer that can benefit from expression of a therapeutic protein in a cancer cell, such as a toxin or thymidine kinase. If the subject is administered two or more synthetic molecules provided herein that express a full-length thymidine kinase, the subject is also administered ganciclovir. Treatment does not require 100% removal of all characteristics of the disorder, but can be a reduction in such. Although specific examples are provided below, based on this teaching one will understand that symptoms of other disorders can be similarly affected. For example, the disclosed methods can be used to increase expression of a protein that is not expressed or has reduced expression by the subject, or decrease expression of a protein that is undesirably expressed or has reduced expression by the subject. For example, the disclosed methods can be used to treat or reduce the undesirable effects of a genetic disease.

For example, the disclosed methods and systems can treat or reduce the undesirable effects of sickle cell disease by expressing a full-length wild-type β-globin chain of hemoglobin. In one example the disclosed methods reduce the symptoms of sickle-cell disease in the recipient subject (such as one or more of, presence of sickle cells in the blood, pain, ischemia, necrosis, anemia, vaso-occlusive crisis, aplastic crisis, splenic sequestration crisis, and haemolytic crisis) for example a reduction of at least 10%, at least 20%, at least 50%, at least 70%, or at least 90% (as compared to no administration of the therapeutic nucleic acid molecule). In one example the disclosed methods decrease the number of sickle cells in the recipient subject, for example a decrease of at least 10%, at least 20%, at least 50%, at least 70%, at least 90%, or at least 95% (as compared to no administration of the therapeutic nucleic acid molecule).

For example, the disclosed methods and systems can treat or reduce the undesirable effects of thrombophilia by expressing a full-length wild-type factor V Leiden or prothrombin gene. In one example the disclosed methods reduce the symptoms of thrombophilia in the recipie7 nt subject (such as one or more of, thrombosis, such as deep vein thrombosis, pulmonary embolism, venous thromboembolism, swelling, chest pain, palpitations) for example a reduction of at least 10%, at least 20%, at least 50%, at least 70%, or at least 90% (as compared to no administration of the therapeutic nucleic acid molecule). In one example the disclosed methods decrease the activity of coagulation factors in the recipient subject, for example a decrease of at least 10%, at least 20%, at least 50%, at least 70%, at least 90%, or at least 95% (as compared to no administration of the therapeutic nucleic acid molecule).

For example, the disclosed methods and systems can treat or reduce the undesirable effects of CD40 ligand deficiency by expressing a full-length wild-type CD40 ligand gene. In one example the disclosed methods reduce the symptoms of CD40 ligand deficiency in the recipient subject (such as one or more of, elevate serum IgM, low serum levels of other immunoglobulins, opportunistic infections, autoimmunity and malignancies) for example a reduction of at least 10%, at least 20%, at least 50%, at least 70%, or at least 90% (as compared to no administration of the therapeutic nucleic acid molecule s). In one example the disclosed methods increase the amount or activity of CD40 ligand deficiency in the recipient subject, for example an increase of at least 10%, at least 20%, at least 50%, at least 70%, at least 90%, at least 100%, at least 200% or at least 500% (as compared to no administration of the therapeutic nucleic acid molecule).

For example, the disclosed methods can be used to treat or reduce the undesirable effects of a primary immunodeficiency disease resulting from a genetic defect. For example, the disclosed methods and systems (which can use two or more synthetic nucleic acid molecules to express a functional protein missing or defective in the subject, for example using AAV) can treat or reduce the undesirable effects of a primary immunodeficiency disease. In one example the disclosed methods reduce the symptoms of a primary immunodeficiency disease in the recipient subject (such as one or more of, a bacterial infection, fungal infection, viral infection, parasitic infection, lymph gland swelling, spleen enlargement, wounds, and weight loss) for example a reduction of at least 10%, at least 20%, at least 50%, at least 70%, or at least 90% (as compared to no administration of the therapeutic nucleic acid molecule). In one example the disclosed methods increase the number of immune cells (such as T cells, such as CD8 cells) in the recipient subject with a primary immune deficiency disorder, for example an increase of at least 10%, at least 20%, at least 50%, at least 70%, at least 90%, at least 95%, at least 100%, at least 200%, at least 300%, at least 400%, or at least 500% (as compared to no administration of the therapeutic nucleic acid molecule). In one example the disclosed methods reduce the number of infections ((such as bacterial, viral, fungal, or combinations thereof) in the recipient subject over a set period of time (such as over 1 year) with a primary immune deficiency disorder, for example a decrease of at least 10%, at least 20%, at least 50%, at least 70%, at least 90%, or at least 95%, (as compared to no administration of the therapeutic nucleic acid molecule).

For example, the disclosed methods can be used to treat or reduce the undesirable effects of a monogenetic disorder. For example, the disclosed methods (which can use two or more synthetic nucleic acid molecules to express a functional protein missing or defective in the subject, for example using AAV) can treat or reduce the undesirable effects of a monogenetic disorder. In one example the disclosed methods reduce the symptoms of a monogenetic disorder in the recipient subject, for example a reduction of at least 10%, at least 20%, at least 50%, at least 70%, or at least 90% (as compared to no administration of the therapeutic nucleic acid molecule). In one example the disclosed methods increase the amount of normal protein not normally expressed by the recipient subject with a monogenetic disorder, for example an increase of at least 10%, at least 20%, at least 50%, at least 70%, at least 90%, at least 95%, at least 100%, at least 200%, at least 300%, at least 400%, or at least 500% (as compared to no administration of the therapeutic nucleic acid molecule).

For example, the disclosed methods can be used to treat or reduce the undesirable effects of a hematological malignancy in the recipient subject. In one example the disclosed methods reduce the number of abnormal white blood cells (such as B cells) in the recipient subject (such as a subject with leukemia), for example a reduction of at least 10%, at least 20%, at least 50%, at least 70%, or at least 90% (as compared to no administration of the disclosed therapies). In one example, administration of the disclosed therapies can be used to treat or reduce the undesirable effects of a lymphoma, such as reduce the size of the lymphoma, volume of the lymphoma, rate of growth of the lymphoma, metastasis of the lymphoma, for example a reduction of at least 10%, at least 20%, at least 50%, at least 70%, or at least 90% (as compared to no administration of the disclosed therapies). In one example, administration of disclosed therapies can be used to treat or reduce the undesirable effects of multiple myeloma, such as reduce the number of abnormal plasma cells in the recipient subject, for example a reduction of at least 10%, at least 20%, at least 50%, at least 70%, or at least 90% (as compared to no administration of the disclosed therapies).

For example, the disclosed methods can be used to treat or reduce the undesirable effects of a malignancy, such as one that results from a genetic defect in the recipient subject. In one example the disclosed methods reduce the number of cancer cells, the size of a tumor, the volume of a tumor, or the number of metastases, in the recipient subject (such as a subject with a cancer listed herein), for example a reduction of at least 10%, at least 20%, at least 50%, at least 70%, or at least 90% (as compared to no administration of the disclosed therapies). In one example, administration of the disclosed therapies can be used to treat or reduce the undesirable effects of a lymphoma, such as reduce the size of the tumor, volume of the tumor, rate of growth of the cancer, metastasis of the cancer, for example a reduction of at least 10%, at least 20%, at least 50%, at least 70%, or at least 90% (as compared to no administration of the disclosed therapies).

For example, the disclosed methods can be used to treat or reduce the undesirable effects of a neurological disease that results from a genetic defect in the recipient subject. In one example the disclosed methods increase neurological function in the recipient subject (such as a subject with a neurological disease listed above), for example an increase of at least 10%, at least 20%, at least 50%, at least 70%, at least 90%, at least 100%, at least 200%, at least 300%, at least 400%, or at least 500% (as compared to no administration of the disclosed therapies).

### VI. Exemplary Embodiments

1. A composition for expressing a nucleic acid editing protein, comprising:
(a) a first RNA molecule comprising from 5′ to 3′:
   (i) a coding sequence for an N-terminal portion of the nucleic acid editing protein;
   (ii) a splice donor; and
   (iii) a first dimerization domain; and
(b) a second RNA molecule comprising from 5′ to 3′:
   (i) a second dimerization domain, wherein the second dimerization domain binds to the first dimerization domain;
   (ii) a branch point sequence;
   (iii) a polypyrimidine tract;
   (iv) a splice acceptor; and
   (v) a coding sequence for a C-terminal portion of the nucleic acid editing protein;
(c) optionally, a third RNA molecule comprising at least one first guide RNA (gRNA) specific for a first target nucleic acid molecule, wherein the at least one first gRNA directs the nucleic acid editing protein to a target editing site on the first target nucleic acid molecule;
(d) optionally, a fourth RNA molecule comprising at least one second gRNA specific for (i) the first target nucleic acid molecule, wherein the at least one second gRNA directs the nucleic acid editing protein to the same or different target editing site on the first nucleic acid molecule, or (ii) a second target nucleic acid molecule, wherein the at least one second gRNA directs the nucleic acid editing protein to a target editing site on the second target nucleic acid molecule;
(e) optionally, a fifth RNA molecule comprising at least one third gRNA specific for (i) the first target nucleic acid molecule, wherein the at least one third gRNA directs the nucleic acid editing protein to the same or different target editing site on the first nucleic acid molecule as the first and second gRNA, (ii) the second target nucleic acid molecule, wherein the at least one third gRNA directs the nucleic acid editing protein to the same or different target editing site on the second nucleic acid molecule as the second gRNA, or (iii) a third target nucleic acid molecule, wherein the at least one third gRNA directs the nucleic acid editing protein to a target editing site on the third target nucleic acid molecule; and
(f) optionally, a sixth RNA molecule comprising at least one fourth gRNA specific for (i) the first target nucleic acid molecule, wherein the at least one fourth gRNA directs the nucleic acid editing protein to the same or different target editing site on the first nucleic acid molecule as the first, second, and third gRNA, (ii) the second target nucleic acid molecule, wherein the at least one fourth gRNA directs the nucleic acid editing protein to the same or different target editing site on the second nucleic acid molecule as the second and third gRNA, (iii) the third target nucleic acid molecule, wherein the at least one fourth gRNA directs the nucleic acid editing protein to the same or different target editing site on the third target nucleic acid molecule as the third gRNA or (iv) a fourth target nucleic acid molecule, wherein the at least one fourth gRNA directs the nucleic acid editing protein to a target editing site on the fourth target nucleic acid molecule.

2. The composition of embodiment 1, wherein the first and second dimerization domains bind by direct binding, indirect binding, or a combination thereof.

3. The composition of embodiment 2, wherein direct binding or indirect binding comprises basepairing interactions, non-canonical base pairing interactions, non-base pairing interactions, or a combination thereof.

4. The composition of embodiment 2 or 3, wherein direct binding comprises base pairing interactions between kissing loops or hypodiverse regions.

5. The composition of embodiment 2 or 3, wherein direct binding comprises non-canonical basepairing interactions, non-canonical base pairing interactions, non-base pairing interactions, or a combination thereof, between aptamer regions.

6. The composition of embodiment 2 or 3, wherein indirect binding comprises basepairing interactions through a nucleic acid bridge.

7. The composition of embodiment 2, wherein indirect binding comprises non-base pairing interactions between an aptamer and an aptamer target, or between two aptamers.

8. The composition of any one of embodiments 1 to 7, wherein the first or second dimerization domain does not comprise a cryptic splice acceptor.

9. The composition of any one of embodiments 1 to 8, wherein the dimerization domains are directly binding or indirectly binding aptamer sequence dimerization domains.

10. The composition of any one of embodiments 1 to 9, wherein the dimerization domains are kissing loop interaction domains.

11. The composition of any one of embodiments 1 to 10, wherein the target editing site is part of a target nucleic acid molecule or a regulatory region of a target nucleic acid molecule associated with disease.

12. The composition of embodiment 11, wherein the disease is a monogenic disease.

13. The composition of any one of embodiments 1 to 12, wherein the first, second, third, and/or fourth target nucleic acid molecule comprises one or more point mutations that results in the disease.

14. The composition of any one of embodiments 11 to 13, wherein the disease and the first, second, third, and/or fourth target nucleic acid molecule are one listed in Tables 1-4.

15. The composition of any one of embodiments 1 to 14, wherein the first RNA molecule further comprises one or both of a downstream intronic splice enhancer (DISE) 3′ to the splice donor and 5′ to the first dimerization domain, an intronic splice enhancer (ISE) 3′ to the splice donor and 5′ to the first dimerization domain; and/or
the second RNA molecule further comprises one or both of an ISE 3′ to the second dimerization domain and 5′ to the branch point sequence, and a DISE 3′ to the splice donor and 5′ to the dimerization domain;
or any combination thereof.

16. The composition of any one of embodiments 1 to 15, wherein
the first RNA molecule further comprises a self-cleaving RNA sequence or an RNA-cleaving enzyme target sequence positioned anywhere 3′ to the splice donor such that it cleaves off the 3′ located polyadenylated tail to decrease or suppress protein fragment expression from a non-recombined RNA molecule;
the second RNA molecule further comprises a self-cleaving RNA sequence or an RNA-cleaving enzyme target sequence positioned anywhere 5′ to the branch point sequence such that it cleaves off the 5′ located RNA cap to decrease or suppress protein fragment expression from a non-recombined RNA molecule;
the second RNA molecule further comprises a start codon anywhere 5′ to the branch point sequence that is shifted relative to the open reading frame 3′ of the splice acceptor to decrease or suppress translation of a nucleic acid editing protein fragment from a non-recombined RNA molecule;
the first RNA molecule further comprises a micro RNA target site anywhere 3′ to the splice donor such that an un-joined RNA fragment undergoes micro RNA dependent degradation once outside the nucleus;
the second RNA molecule further comprises a micro RNA target site anywhere 3′ to the coding sequence such that an un-joined RNA fragment undergoes micro RNA dependent degradation once outside the nucleus;
the first RNA molecule further comprises a sequence encoding a degron protein degradation tag anywhere 3′ to the splice donor such that it is in frame with the nucleic acid editing protein open reading frame 5′ of the splice donor site such that an un-joined protein fragment is tagged for degradation;
the second RNA molecule further comprises a start codon and an in-frame degron protein degradation tag anywhere 5′ to the branch point sequence such that it is in frame with the nucleic acid editing protein open reading frame 3′ of the splice acceptor site such that an un-joined protein fragment is tagged for degradation;
or any combination thereof.

17. The composition of any one of embodiments 1-16, wherein the nucleic acid editing protein comprises a Cas nuclease, zinc finger nuclease, or transcription activator-like effector nuclease.

18. The composition of any one of embodiments 1-17, wherein the first, second, third, and/or fourth target nucleic acid molecule are target DNA molecules, and the at least one first, second, third, and/or fourth gRNA comprises a crRNA and tracrRNA

19. The composition of embodiment 18, wherein the nucleic acid editing protein comprises Cas9 or dead Cas9 (dCas9).

20. The composition of embodiment 19, wherein
the Cas9 protein comprises at least 80%, at least 85%, at least 90%, at least 95%, at least 96%, at least 97%, at least 98%, at least 99%, or 100% sequence identity to SEQ ID NO: 208, and can function as an RNA-guided DNA endonuclease;
the Cas9 protein is encoded by a sequence comprising at least 80%, at least 85%, at least 90%, at least 95%, at least 96%, at least 97%, at least 98%, at least 99%, or 100% sequence identity to SEQ ID NO: 207, and encodes an RNA-guided DNA endonuclease;
the dCas9 protein comprises at least 80%, at least 85%, at least 90%, at least 95%, at least 96%, at least 97%, at least 98%, at least 99%, or 100% sequence identity to SEQ ID NO: 210, and is catalytically inactive;
the dCas9 protein is encoded by a sequence comprising at least 80%, at least 85%, at least 90%, at least 95%, at least 96%, at least 97%, at least 98%, at least 99%, or 100% sequence identity to SEQ ID NO: 209, and encodes a protein that is catalytically inactive.

21. The composition of embodiment 19 or 20, wherein the Cas9 or dCas9 is part of a fusion protein.

22. The composition of embodiment 21, wherein the fusion protein comprises Cas9 or dCas9 and one or more of:
a transcriptional activation domain, such as VP64, P65, MyoD1, HSF1, RTA, CBP, SET7/9, or any combination thereof;
a cytosine base editor (CBE), such as one from sea lamprey [AID], CDA1, or APOBEC3G;
bacteriophage protein Gam; and
an adenine base editor (ABE), such as ABE 6.3, 7.8, 7.9, 7.10, ABEmax, ABE8s, such as ABE8e(TadA-8e V106W).

23. The composition of any one of embodiments 1-17, wherein the first, second, third, and/or fourth target nucleic acid molecule are target RNA, and the at least one first, second, third, and/or fourth gRNA comprises one or more direct repeats and one or more spacers.

24. The composition of embodiment 23, wherein the nucleic acid editing protein comprises Cas13a, Cas13b, Cas13c, Cas13d or dead Cas13d (dCas13d).

25. The composition of embodiment 24, wherein
the Cas13d protein comprises at least 80%, at least 85%, at least 90%, at least 95%, at least 96%, at least 97%, at least 98%, at least 99%, or 100% sequence identity to SEQ ID NO: 212, 215, or 222, and can function as an RNA-guided RNA endonuclease;
the Cas13d protein is encoded by a sequence comprising at least 80%, at least 85%, at least 90%, at least 95%, at least 96%, at least 97%, at least 98%, at least 99%, or 100% sequence identity to SEQ ID NO: 211 or 213, and encodes an RNA-guided RNA endonuclease;
the dCas13d protein comprises at least 80%, at least 85%, at least 90%, at least 95%, at least 96%, at least 97%, at least 98%, at least 99%, or 100% sequence identity to SEQ ID NO: 215 or 216, and is catalytically inactive;
the dCas13d protein is encoded by a sequence that encodes a protein comprising at least 80%, at least 85%, at least 90%, at least 95%, at least 96%, at least 97%, at least 98%, at least 99%, or 100% sequence identity to SEQ ID NO: 215 or 216, and encodes a protein that is catalytically inactive.

26. The composition of embodiment 24 or 25, wherein the Cas13a, Cas13b, Cas13c, Cas13d or dCas13d is part of a fusion protein.

27. The composition of embodiment 26, wherein the fusion protein comprises Cas13a, Cas13b, Cas13c, Cas13d or dCas13d and one or more of:
a transcriptional activation domain, such as VP64, P65, MyoDI, HSF1, RTA, CBP, SET7/9, or any combination thereof;
a cytosine base editor (CBE), such as one from sea lamprey [AID], CDA1, or APOBEC3G;
bacteriophage protein Gam: and
an adenine base editor (ABE), such as ABE 6.3, 7.8, 7.9, 7.10, ABEmax, ABE8s, such as ABE8e(TadA-8e V106W).

28. The composition of any one of embodiments 1-27, wherein the at least one first, second, third, and/or fourth gRNA comprise multiple copies of each of the first, second, third, and/or fourth gRNA.

29. The composition of any one of embodiments 1-27, further comprising a parvovirus inverted terminal repeat (ITR) at the 5′ and the 3′-end of each of the first RNA molecule and the second RNA molecule.

30. A DNA molecule composition for expressing the composition of any one of embodiments 1-29, comprising:
(i) a first synthetic DNA molecule encoding the RNA molecules of (a), and optionally (c), (d), or both (c) and (d); and
(ii) a second synthetic DNA molecule encoding the RNA molecule of (b), and optionally and (e), (f), or both (e) and (f).

31. The DNA molecule of embodiment 30, further comprising:
(a) a first promoter operably linked to a DNA sequence encoding the N-terminal portion of the nucleic acid editing protein;
(b) a second promoter operably linked to a DNA sequence encoding the C-terminal portion of the nucleic acid editing protein
(c) if the first synthetic DNA molecule includes a DNA molecule encoding the RNA molecule of (c), a third promoter operably linked to a sequence encoding the at least one first gRNA;
(d) if the first synthetic DNA molecule includes a DNA molecule encoding the RNA molecule of (d), a fourth promoter operably linked to a sequence encoding the at least one second gRNA;
(e) if the second synthetic DNA molecule includes a DNA molecule encoding the RNA molecule of (e), a fifth promoter operably linked to a sequence encoding the at least one third gRNA; and
(f) if the second synthetic DNA molecule includes a DNA molecule encoding the RNA molecule of (d), a sixth promoter operably linked to a sequence encoding the at least one fourth gRNA

32. The composition of embodiment 31, wherein each promoter is independently selected.

33. The composition of embodiment 31 or 32, wherein:
the first and second promoter are the same promoter;
the first and second promoter are different promoters,
the third, fourth, fifth and sixth promoters are the same promoter;
the third, fourth, fifth and sixth promoters are different promoters, or combinations thereof.

34. The composition of any one of embodiments 30 to 32, wherein each of the first and second promoters is independently selected from: a constitutive promoter; a tissue-specific promoter; and a promoter endogenous to the nucleic acid editing protein.

35. The composition of any one of embodiments 30 to 34, wherein each of the third, fourth, fifth and sixth promoters is a polymerase III promoter, such as a U6 or H1 promoter.

36. A system for expressing a nucleic acid editing protein comprising a composition of any one of embodiments 30 to 35.

37. The system of embodiment 36, wherein when the system is introduced into a cell the RNA molecules are produced and recombine in the proper order, resulting in a full-length coding sequence of the nucleic acid editing protein.

38. The system of embodiment 36 or 37, wherein each of the synthetic first and second RNA molecules are transcribed from a separate viral vector.

39. The system of embodiment 38, wherein the viral vector is AAV.

40. The system of any one of embodiments 36 to 39, wherein each of the synthetic DNA molecules has a size independently selected from: about 2500 nt to about 5000 nt, 2,500 nt to about 2,750 nt, about 2,500 nt to about 3,000 nt, about 2,500 nt to about 3,250 nt, about 2,500 nt to about 3,500 nt, about 2,500 nt to about 3,750 nt, about 2,500 nt to about 4,000 nt, about 2,500 nt to about 4,250 nt, about 2,500 nt to about 4,500 nt, about 2,500 nt to about 4,750 nt, about 2,500 nt to about 5,000 nt, about 2,750 nt to about 3,000 nt, about 2,750 nt to about 3,250 nt, about 2,750 nt to about 3,500 nt, about 2,750 nt to about 3,750 nt, about 2,750 nt to about 4,000 nt, about 2,750 nt to about 4,250 nt, about 2,750 nt to about 4,500 nt, about 2,750 nt to about 4,750 nt, about 2,750 nt to about 5,000 nt, about 3,000 nt to about 3,250 nt, about 3,000 nt to about 3,500 nt, about 3,000 nt to about 3,750 nt, about 3,000 nt to about 4,000 nt, about 3,000 nt to about 4,250 nt, about 3,000 nt to about 4,500 nt, about 3,000 nt to about 4,750 nt, about 3,000 nt to about 5,000 nt, about 3,250 nt to about 3,500 nt, about 3,250 nt to about 3,750 nt, about 3,250 nt to about 4,000 nt, about 3,250 nt to about 4,250 nt, about 3,250 nt to about 4,500 nt, about 3,250 nt to about 4,750 nt, about 3,250 nt to about 5,000 nt, about 3,500 nt to about 3,750 nt, about 3,500 nt to about 4,000 nt, about 3,500 nt to about 4,250 nt, about 3,500 nt to about 4,500 nt, about 3,500 nt to about 4,750 nt, about 3,500 nt to about 5,000 nt, about 3,750 nt to about 4,000 nt, about 3,750 nt to about 4,250 nt, about 3,750 nt to about 4,500 nt, about 3,750 nt to about 4,750 nt, about 3,750 nt to about 5,000 nt, about 4,000 nt to about 4,250 nt, about 4,000 nt to about 4,500 nt, about 4,000 nt to about 4,750 nt, about 4,000 nt to about 5,000 nt, about 4,250 nt to about 4,500 nt, about 4,250 nt to about 4,750 nt, about 4,250 nt to about 5,000 nt, about 4,500 nt to about 4,750 nt, about 4,500 nt to about 5,000 nt, about 4,750 nt to about 5,000 nt, about 2,500 nt, about 2,750 nt, about 3,000 nt, about 3,250 nt, about 3,500 nt, about 3,750 nt, about 4,000 nt, about 4,250 nt, about 4,500 nt, about 4,750 nt, and about 5,000 nt.

41. The system of any one of embodiments 36 to 40, wherein the coding sequence for an N-terminal portion of the nucleic acid editing protein, or a C-terminal portion of the nucleic acid editing protein encoded by a synthetic DNA molecule of the system each has a size independently selected from: about 2500 to 4500 nt, about 2,500 nt to about 2,750 nt, about 2,500 nt to about 3,000 nt, about 2,500 nt to about 3,250 nt, about 2,500 nt to about 3,500 nt, about 2,500 nt to about 3,750 nt, about 2,500 nt to about 4,000 nt, about 2,500 nt to about 4,250 nt, about 2,500 nt to about 4,500 nt, about 2,750 nt to about 3,000 nt, about 2,750 nt to about 3,250 nt, about 2,750 nt to about 3,500 nt, about 2,750 nt to about 3,750 nt, about 2,750 nt to about 4,000 nt, about 2,750 nt to about 4,250 nt, about 2,750 nt to about 4,500 nt, about 3,000 nt to about 3,250 nt, about 3,000 nt to about 3,500 nt, about 3,000 nt to about 3,750 nt, about 3,000 nt to about 4,000 nt, about 3,000 nt to about 4,250 nt, about 3,000 nt to about 4,500 nt, about 3,250 nt to about 3,500 nt, about 3,250 nt to about 3,750 nt, about 3,250 nt to about 4,000 nt, about 3,250 nt to about 4,250 nt, about 3,250 nt to about 4,500 nt, about 3,500 nt to about 3,750 nt, about 3,500 nt to about 4,000 nt, about 3,500 nt to about 4,250 nt, about 3,500 nt to about 4,500 nt, about 3,750 nt to about 4,000 nt, about 3,750 nt to about 4,250 nt, about 3,750 nt to about 4,500 nt, about 4,000 nt to about 4,250 nt, about 4,000 nt to about 4,500 nt, about 4,250 nt to about 4,500 nt, about 2,500 nt, about 2,750 nt, about 3,000 nt, about 3,250 nt, about 3,500 nt, about 3,750 nt, about 4,000 nt, about 4,250 nt, and about 4,500 nt.

42. The system of any one of embodiments 36 to 41, wherein any one or both of the RNA molecules encoded by the synthetic DNA molecules of the system, respectively, has a size independently selected from: about 2500 to 4500 nt, about 2,500 nt to about 2,750 nt, about 2,500 nt to about 3,000 nt, about 2,500 nt to about 3,250 nt, about 2,500 nt to about 3,500 nt, about 2,500 nt to about 3,750 nt, about 2,500 nt to about 4,000 nt, about 2,500 nt to about 4,250 nt, about 2,500 nt to about 4,500 nt, about 2,750 nt to about 3,000 nt, about 2,750 nt to about 3,250 nt, about 2,750 nt to about 3,500 nt, about 2,750 nt to about 3,750 nt, about 2,750 nt to about 4,000 nt, about 2,750 nt to about 4,250 nt, about 2,750 nt to about 4,500 nt, about 3,000 nt to about 3,250 nt, about 3,000 nt to about 3,500 nt, about 3,000 nt to about 3,750 nt, about 3,000 nt to about 4,000 nt, about 3,000 nt to about 4,250 nt, about 3,000 nt to about 4,500 nt, about 3,250 nt to about 3,500 nt, about 3,250 nt to about 3,750 nt, about 3,250 nt to about 4,000 nt, about 3,250 nt to about 4,250 nt, about 3,250 nt to about 4,500 nt, about 3,500 nt to about 3,750 nt, about 3,500 nt to about 4,000 nt, about 3,500 nt to about 4,250 nt, about 3,500 nt to about 4,500 nt, about 3,750 nt to about 4,000 nt, about 3,750 nt to about 4,250 nt, about 3,750 nt to about 4,500 nt, about 4,000 nt to about 4,250 nt, about 4,000 nt to about 4,500 nt, about 4,250 nt to about 4,500 nt, about 2,500 nt, about 2,750 nt, about 3,000 nt, about 3,250 nt, about 3,500 nt, about 3,750 nt, about 4,000 nt, about 4,250 nt, and about 4,500 nt.

43. The system of any one of embodiments 36 to 42, wherein the system comprises a composition of any one of embodiments 30 to 35:
the synthetic DNA molecules have a total size selected from about 5000 nt to about 10,000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,000 nt to about 8,500 nt, about 5,000 nt to about 9,000 nt, about 5,000 nt to about 9,500 nt, about 5,000 nt to about 10,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 5,500 nt to about 8,500 nt, about 5,500 nt to about 9,000 nt, about 5,500 nt to about 9,500 nt, about 5,500 nt to about 10,000 nt, about 6,000 nt to about 6,500 nt, about 6,000 nt to about 7,000 nt, about 6,000 nt to about 7,500 nt, about 6,000 nt to about 8,000 nt, about 6,000 nt to about 8,500 nt, about 6,000 nt to about 9,000 nt, about 6,000 nt to about 9,500 nt, about 6,000 nt to about 10,000 nt, about 6,500 nt to about 7,000 nt, about 6,500 nt to about 7,500 nt, about 6,500 nt to about 8,000 nt, about 6,500 nt to about 8,500 nt, about 6,500 nt to about 9,000 nt, about 6,500 nt to about 9,500 nt, about 6,500 nt to about 10,000 nt, about 7,000 nt to about 7,500 nt, about 7,000 nt to about 8,000 nt, about 7,000 nt to about 8,500 nt, about 7,000 nt to about 9,000 nt, about 7,000 nt to about 9,500 nt, about 7,000 nt to about 10,000 nt, about 7,500 nt to about 8,000 nt, about 7,500 nt to about 8,500 nt, about 7,500 nt to about 9,000 nt, about 7,500 nt to about 9,500 nt, about 7,500 nt to about 10,000 nt, about 8,000 nt to about 8,500 nt, about 8,000 nt to about 9,000 nt, about 8,000 nt to about 9,500 nt, about 8,000 nt to about 10,000 nt, about 8,500 nt to about 9,000 nt, about 8,500 nt to about 9,500 nt, about 8,500 nt to about 10,000 nt, about 9,000 nt to about 9,500 nt, about 9,000 nt to about 10,000 nt, about 9,500 nt to about 10,000 nt, about 5,000 nt, about 5,500 nt, about 6,000 nt, about 6,500 nt, about 7,000 nt, about 7,500 nt, about 8,000 nt, about 8,500 nt, about 9,000 nt, about 9,500 nt, and about 10,000 nt;
the total nucleic acid editing protein coding sequence is selected from about 2000 nt to about 8000 nt, about 2,000 nt to about 3,000 nt, about 2,000 nt to about 3,500 nt, about 2,000 nt to about 4,000 nt, about 2,000 nt to about 4,500 nt, about 2,000 nt to about 5,000 nt, about 2,000 nt to about 5,500 nt, about 2,000 nt to about 6,000 nt, about 2,000 nt to about 6,500 nt, about 2,000 nt to about 7,000 nt, about 2,000 nt to about 7,500 nt, about 2,000 nt to about 8,000 nt, about 3,000 nt to about 3,500 nt, about 3,000 nt to about 4,000 nt, about 3,000 nt to about 4,500 nt, about 3,000 nt to about 5,000 nt, about 3,000 nt to about 5,500 nt, about 3,000 nt to about 6,000 nt, about 3,000 nt to about 6,500 nt, about 3,000 nt to about 7,000 nt, about 3,000 nt to about 7,500 nt, about 3,000 nt to about 8,000 nt, about 3,500 nt to about 4,000 nt, about 3,500 nt to about 4,500 nt, about 3,500 nt to about 5,000 nt, about 3,500 nt to about 5,500 nt, about 3,500 nt to about 6,000 nt, about 3,500 nt to about 6,500 nt, about 3,500 nt to about 7,000 nt, about 3,500 nt to about 7,500 nt, about 3,500 nt to about 8,000 nt, about 4,000 nt to about 4,500 nt, about 4,000 nt to about 5,000 nt, about 4,000 nt to about 5,500 nt, about 4,000 nt to about 6,000 nt, about 4,000 nt to about 6,500 nt, about 4,000 nt to about 7,000 nt, about 4,000 nt to about 7,500 nt, about 4,000 nt to about 8,000 nt, about 4,500 nt to about 5,000 nt, about 4,500 nt to about 5,500 nt, about 4,500 nt to about 6,000 nt, about 4,500 nt to about 6,500 nt, about 4,500 nt to about 7,000 nt, about 4,500 nt to about 7,500 nt, about 4,500 nt to about 8,000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 6,000 nt to about 6,500 nt, about 6,000 nt to about 7,000 nt, about 6,000 nt to about 7,500 nt, about 6,000 nt to about 8,000 nt, about 6,500 nt to about 7,000 nt, about 6,500 nt to about 7,500 nt, about 6,500 nt to about 8,000 nt, about 7,000 nt to about 7,500 nt, about 7,000 nt to about 8,000 nt, or about 7,500 nt to about 8,000 nt, the total target protein coding sequence is about 2,000 nt, about 3,000 nt, about 3,500 nt, about 4,000 nt, about 4,500 nt, about 5,000 nt, about 5,500 nt, about 6,000 nt, about 6,500 nt, about 7,000 nt, about 7,500 nt, and about 8,000 nt; and/or
the summed size of the RNA molecules encoded by the two synthetic DNA molecules is selected from about 5,000 nt to about 9000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,000 nt to about 8,500 nt, about 5,000 nt to about 9,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 5,500 nt to about 8,500 nt, about 5,500 nt to about 9,000 nt, about 6,000 nt to about 6,500 nt, about 6,000 nt to about 7,000 nt, about 6,000 nt to about 7,500 nt, about 6,000 nt to about 8,000 nt, about 6,000 nt to about 8,500 nt, about 6,000 nt to about 9,000 nt, about 6,500 nt to about 7,000 nt, about 6,500 nt to about 7,500 nt, about 6,500 nt to about 8,000 nt, about 6,500 nt to about 8,500 nt, about 6,500 nt to about 9,000 nt, about 7,000 nt to about 7,500 nt, about 7,000 nt to about 8,000 nt, about 7,000 nt to about 8,500 nt, about 7,000 nt to about 9,000 nt, about 7,500 nt to about 8,000 nt, about 7,500 nt to about 8,500 nt, about 7,500 nt to about 9,000 nt, about 8,000 nt to about 8,500 nt, about 8,000 nt to about 9,000 nt, about 8,500 nt to about 9,000 nt, about 5,000 nt, about 5,500 nt, about 6,000 nt, about 6,500 nt, about 7,000 nt, about 7,500 nt, about 8,000 nt, about 8,500 nt, and about 9,000 nt.

44. The system of any one of embodiments 36 to 43, wherein the first dimerization domain and the second dimerization domain are each no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150, 200, 250, 300, 400, or 500 nt; and the system has a recombination efficiency of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

45. The system of any one of embodiments 36 to 44, wherein each dimerization domain is no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150, 200, 250, 300, 400, or 500 nt; and the system has a recombination efficiency of at least 20%, at least 30%, at least 40%, at least 50%, at least 60%, at least 70%, at least 75%, at least 80%, at least 90%, or about 100%.

46. The system of any one of embodiments 36 to 45, wherein the RNA recombination efficiency is about 10% to about 100%, about 10% to about 20%, about 10% to about 30%, about 10% to about 35%, about 10% to about 40%, about 10% to about 45%, about 10% to about 50%, about 10% to about 55%, about 10% to about 60%, about 10% to about 70%, about 10% to about 80%, about 10% to about 90%, about 20% to about 30%, about 20% to about 35%, about 20% to about 40%, about 20% to about 45%, about 20% to about 50%, about 20% to about 55%, about 20% to about 60%, about 20% to about 70%, about 20% to about 80%, about 20% to about 90%, about 30% to about 35%, about 30% to about 40%, about 30% to about 45%, about 30% to about 50%, about 30% to about 55%, about 30% to about 60%, about 30% to about 70%, about 30% to about 80%, about 30% to about 90%, about 35% to about 40%, about 35% to about 45%, about 35% to about 50%, about 35% to about 55%, about 35% to about 60%, about 35% to about 70%, about 35% to about 80%, about 35% to about 90%, about 40% to about 45%, about 40% to about 50%, about 40% to about 55%, about 40% to about 60%, about 40% to about 70%, about 40% to about 80%, about 40% to about 90%, about 45% to about 50%, about 45% to about 55%, about 45% to about 60%, about 45% to about 70%, about 45% to about 80%, about 45% to about 90%, about 50% to about 55%, about 50% to about 60%, about 50% to about 70%, about 50% to about 80%, about 50% to about 90%, about 55% to about 60%, about 55% to about 70%, about 55% to about 80%, about 55% to about 90%, about 60% to about 70%, about 60% to about 80%, about 60% to about 90%, about 70% to about 80%, about 70% to about 90%, about 80% to about 90%, about 10%, about 20%, about 30%, about 35%, about 40%, about 45%, about 50%, about 55%, about 60%, about 70%, about 80%, about 90%, about 95%, or about 100%.

47. A composition comprising a system of any one of embodiments 36 to 46.

48. The composition of embodiment 47, wherein the composition comprises first, second, third and optionally fourth RNA molecules, each encoding at least a portion of a nucleic acid editing protein.

49. A kit comprising the system of any one of embodiments 36 to 46, or composition of any one of embodiments 47 and 48, wherein any of the synthetic first, second, third and fourth nucleic acid molecules can be in separate containers, and optionally further comprising a buffer such as a pharmaceutically acceptable carrier.

50. A method of expressing a nucleic acid editing protein in a cell, comprising:
introducing the system of any one of embodiments 36 to 46, or a composition of embodiment 47 or 48, into a cell, and expressing the first and second RNA molecules in the cell, wherein the nucleic acid editing protein is produced in the cell.

51. The method of embodiment 50, wherein the cell is in a subject, and introducing comprises administering a therapeutically effective amount the system to the subject.

52. The method of embodiment 51, wherein the method treats a genetic disease caused by a mutation in a target nucleic acid molecule in the subject, wherein the method results in expression of the nucleic acid editing protein and optionally the first and second gRNAs and correction of the mutation in the subject.

53. The method of embodiment 52, wherein
the genetic disease is one resulting from loss of function mutation, and the at least one first, second, third and/or fourth gRNA comprise a sequence that targets a nucleic acid listed in Table 1, and the method treats the corresponding disease listed in Table 1;
the target nucleic acid is an oncogene, and least one first, second, third and/or fourth gRNA comprise a sequence that targets a an oncogene in Table 2 and the method treat the corresponding cancer listed in Table 2;
the genetic disease is one resulting from gain of function mutation, and the at least one first, second, third and/or fourth gRNA comprise a sequence that targets a nucleic acid listed in Table 3, and the method treats the corresponding disease listed in Table 3; or the genetic disease is one listed in Table 4, wherein at least one first, second, third and/or fourth gRNA comprise a sequence targets a nucleic acid listed in Table 4, and the method treats the corresponding disease listed in Table 4.

54. A system of any one of embodiments 36 to 46, a composition of any one of embodiments 1 to 35, 47 and 48, or a method of any one of embodiments 50 to 53, wherein one or both of the first and second RNA molecules comprise at least 80%, at least 85%, at least 90%, at least 95%, at least 98%, at least 99%, or 100% sequence identity to a synthetic intron provided in any one of SEQ ID NOS: 159, 160, 161, 162, 163, 164, 165, 166, 225, and 226.

55. A system of any one of embodiments 36 to 46 and 54, a composition of any one of embodiments 1 to 35, 47 and 48, or a method of any one of embodiments 50 to 53, wherein one or both of the first and second RNA molecules comprise a synthetic intron selected from the RNA encoded by SEQ ID NO: 159, 160, 161, 162, 163, 164, 165, 166, 225, or 226.

56. A system of any of embodiments 36 to 46, 54, and 55, a composition of any one of embodiments 1 to 16, 32 and 33, or a method of any one of embodiments 35 to 38, wherein one or both of the first and second RNA molecules further comprise a portion of a protein coding sequence.

57. A system of any of embodiments 36 to 46 and 54 to 56, a composition of any one of embodiments 1 to 35, 47 and 48, or a method of any one of embodiments 50 to 53, wherein the portion of the protein coding sequence comprises an N-terminal half, an N-terminal portion, a C-terminal half, or a C-terminal portion, of the protein coding sequence.

57. A system of any one of embodiments 36 to 46 and 54 to 56, a composition of any one of embodiments 1 to 35, 47 and 48, or a method of any one of embodiments 50 to 53, comprising: (a) a first RNA molecule, the RNA molecule comprising from 5′ to 3′: (i) at least one first gRNA, (ii) a coding sequence for an N-terminal portion of the nucleic acid editing protein; (iii) a splice donor; (ii-2) a DISE, an ISE, or both; (iv) a first dimerization domain; and (v) at least one second gRNA, and (b) a second RNA molecule, the RNA molecule comprising from 5′ to 3′: (i) at least one third gRNA, (ii) a second dimerization domain, wherein the second dimerization domain binds to the first dimerization domain; (i-2) at least one ISE sequences; (iii) a branch point sequence; (ii) a polypyrimidine tract; (v) a splice acceptor; (vi) a coding sequence for a C-terminal portion of the nucleic acid editing protein, and (vii) at least one fourth gRNA.

58. A system of any one of embodiments 36 to 46 and 54 to 57, a composition of any one of embodiments 1 to 35, 47 and 48, or a method of any one of embodiments 50 to 53, comprising: (a) a first RNA molecule, the RNA molecule comprising from 5′ to 3′: (i) at least one first gRNA, (ii) a coding sequence for an N-terminal portion of the nucleic acid editing protein; (iii) a splice donor; (ii-2) a DISE, an ISE, and an ISE; and (iv) a first dimerization domain; and (v) at least one second gRNA, and (b) a second RNA molecule, the RNA molecule comprising from 5′ to 3′: (i) at least one third gRNA, (ii) a second dimerization domain, wherein the second dimerization domain binds to the first dimerization domain; (i-2) three ISE sequences; (iii) a branch point sequence; (iv) a polypyrimidine tract; (v) a splice acceptor; (vi) a coding sequence for a C-terminal portion of the nucleic acid editing protein and (vii) at least one fourth gRNA.

59. The composition of any one of embodiments 1 to 35, wherein any one or two of the first and second RNA molecules each has a size independently selected from: about 2500 to 4500 nt, about 2,500 nt to about 2,750 nt, about 2,500 nt to about 3,000 nt, about 2,500 nt to about 3,250 nt, about 2,500 nt to about 3,500 nt, about 2,500 nt to about 3,750 nt, about 2,500 nt to about 4,000 nt, about 2,500 nt to about 4,250 nt, about 2,500 nt to about 4,500 nt, about 2,750 nt to about 3,000 nt, about 2,750 nt to about 3,250 nt, about 2,750 nt to about 3,500 nt, about 2,750 nt to about 3,750 nt, about 2,750 nt to about 4,000 nt, about 2,750 nt to about 4,250 nt, about 2,750 nt to about 4,500 nt, about 3,000 nt to about 3,250 nt, about 3,000 nt to about 3,500 nt, about 3,000 nt to about 3,750 nt, about 3,000 nt to about 4,000 nt, about 3,000 nt to about 4,250 nt, about 3,000 nt to about 4,500 nt, about 3,250 nt to about 3,500 nt, about 3,250 nt to about 3,750 nt, about 3,250 nt to about 4,000 nt, about 3,250 nt to about 4,250 nt, about 3,250 nt to about 4,500 nt, about 3,500 nt to about 3,750 nt, about 3,500 nt to about 4,000 nt, about 3,500 nt to about 4,250 nt, about 3,500 nt to about 4,500 nt, about 3,750 nt to about 4,000 nt, about 3,750 nt to about 4,250 nt, about 3,750 nt to about 4,500 nt, about 4,000 nt to about 4,250 nt, about 4,000 nt to about 4,500 nt, about 4,250 nt to about 4,500 nt, about 2,500 nt, about 2,750 nt, about 3,000 nt, about 3,250 nt, about 3,500 nt, about 3,750 nt, about 4,000 nt, about 4,250 nt, and about 4,500 nt.

60. The composition of any one of embodiments 1 to 35, wherein:
the total nucleic acid editing protein coding sequence size is about 2000 nt to about 8000 nt, about 2,000 nt to about 3,000 nt, about 2,000 nt to about 3,500 nt, about 2,000 nt to about 4,000 nt, about 2,000 nt to about 4,500 nt, about 2,000 nt to about 5,000 nt, about 2,000 nt to about 5,500 nt, about 2,000 nt to about 6,000 nt, about 2,000 nt to about 6,500 nt, about 2,000 nt to about 7,000 nt, about 2,000 nt to about 7,500 nt, about 2,000 nt to about 8,000 nt, about 3,000 nt to about 3,500 nt, about 3,000 nt to about 4,000 nt, about 3,000 nt to about 4,500 nt, about 3,000 nt to about 5,000 nt, about 3,000 nt to about 5,500 nt, about 3,000 nt to about 6,000 nt, about 3,000 nt to about 6,500 nt, about 3,000 nt to about 7,000 nt, about 3,000 nt to about 7,500 nt, about 3,000 nt to about 8,000 nt, about 3,500 nt to about 4,000 nt, about 3,500 nt to about 4,500 nt, about 3,500 nt to about 5,000 nt, about 3,500 nt to about 5,500 nt, about 3,500 nt to about 6,000 nt, about 3,500 nt to about 6,500 nt, about 3,500 nt to about 7,000 nt, about 3,500 nt to about 7,500 nt, about 3,500 nt to about 8,000 nt, about 4,000 nt to about 4,500 nt, about 4,000 nt to about 5,000 nt, about 4,000 nt to about 5,500 nt, about 4,000 nt to about 6,000 nt, about 4,000 nt to about 6,500 nt, about 4,000 nt to about 7,000 nt, about 4,000 nt to about 7,500 nt, about 4,000 nt to about 8,000 nt, about 4,500 nt to about 5,000 nt, about 4,500 nt to about 5,500 nt, about 4,500 nt to about 6,000 nt, about 4,500 nt to about 6,500 nt, about 4,500 nt to about 7,000 nt, about 4,500 nt to about 7,500 nt, about 4,500 nt to about 8,000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 6,000 nt to about 6,500 nt, about 6,000 nt to about 7,000 nt, about 6,000 nt to about 7,500 nt, about 6,000 nt to about 8,000 nt, about 6,500 nt to about 7,000 nt, about 6,500 nt to about 7,500 nt, about 6,500 nt to about 8,000 nt, about 7,000 nt to about 7,500 nt, about 7,000 nt to about 8,000 nt, about 7,500 nt to about 8,000 nt, about 2,000 nt, about 3,000 nt, about 3,500 nt, about 4,000 nt, about 4,500 nt, about 5,000 nt, about 5,500 nt, about 6,000 nt, about 6,500 nt, about 7,000 nt, about 7,500 nt, or about 8,000 nt; and/or
the summed size of the two RNA molecules is about 5,000 nt to about 9000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,000 nt to about 8,500 nt, about 5,000 nt to about 9,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 5,500 nt to about 8,500 nt, about 5,500 nt to about 9,000 nt, about 6,000 nt to about 6,500 nt, about 6,000 nt to about 7,000 nt, about 6,000 nt to about 7,500 nt, about 6,000 nt to about 8,000 nt, about 6,000 nt to about 8,500 nt, about 6,000 nt to about 9,000 nt, about 6,500 nt to about 7,000 nt, about 6,500 nt to about 7,500 nt, about 6,500 nt to about 8,000 nt, about 6,500 nt to about 8,500 nt, about 6,500 nt to about 9,000 nt, about 7,000 nt to about 7,500 nt, about 7,000 nt to about 8,000 nt, about 7,000 nt to about 8,500 nt, about 7,000 nt to about 9,000 nt, about 7,500 nt to about 8,000 nt, about 7,500 nt to about 8,500 nt, about 7,500 nt to about 9,000 nt, about 8,000 nt to about 8,500 nt, about 8,000 nt to about 9,000 nt, about 8,500 nt to about 9,000 nt, about 5,000 nt, about 5,500 nt, about 6,000 nt, about 6,500 nt, about 7,000 nt, about 7,500 nt, about 8,000 nt, about 8,500 nt, or about 9,000 nt.

61. The composition of any one of embodiments 1 to 35, wherein the first dimerization domain and the second dimerization domain are each no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150, 200, 250, 300, 400, or 500 nt; and the system has a recombination efficiency of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

62. The composition of any one of embodiments 1 to 35, wherein each dimerization domain is no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150. 200, 250, 300, 400, or 500 nt; and the system has a recombination efficiency of at least 20%, at least 30%, at least 40%, at least 50%, at least 60%, at least 70%, at least 75%, at least 80%, or at least 90%.

63. The composition of any one of embodiments 1 to 35, wherein the RNA recombination efficiency is about 10% to about 100%, about 10% to about 20%, about 10% to about 30%, about 10% to about 35%, about 10% to about 40%, about 10% to about 45%, about 10% to about 50%, about 10% to about 55%, about 10% to about 60%, about 10% to about 70%, about 10% to about 80%, about 10% to about 90%, about 20% to about 30%, about 20% to about 35%, about 20% to about 40%, about 20% to about 45%, about 20% to about 50%, about 20% to about 55%, about 20% to about 60%, about 20% to about 70%, about 20% to about 80%, about 20% to about 90%, about 30% to about 35%, about 30% to about 40%, about 30% to about 45%, about 30% to about 50%, about 30% to about 55%, about 30% to about 60%, about 30% to about 70%, about 30% to about 80%, about 30% to about 90%, about 35% to about 40%, about 35% to about 45%, about 35% to about 50%, about 35% to about 55%, about 35% to about 60%, about 35% to about 70%, about 35% to about 80%, about 35% to about 90%, about 40% to about 45%, about 40% to about 50%, about 40% to about 55%, about 40% to about 60%, about 40% to about 70%, about 40% to about 80%, about 40% to about 90%, about 45% to about 50%, about 45% to about 55%, about 45% to about 60%, about 45% to about 70%, about 45% to about 80%, about 45% to about 90%, about 50% to about 55%, about 50% to about 60%, about 50% to about 70%, about 50% to about 80%, about 50% to about 90%, about 55% to about 60%, about 55% to about 70%, about 55% to about 80%, about 55% to about 90%, about 60% to about 70%, about 60% to about 80%, about 60% to about 90%, about 70% to about 80%, about 70% to about 90%, about 80% to about 90%, about 10%, about 20%, about 30%, about 35%, about 40%, about 45%, about 50%, about 55%, about 60%, about 70%, about 80%, about 90%, about 95%, or about 100%.

64. The composition of any one of embodiments 1 to 35, wherein:
(a) the first and second RNA molecules are each about 2500 nt to 4500 nt;
(b) the total nucleci acid editing protein coding sequence size is about 2000 nt to about 8000 nt; and/or
(c) the summed size of the two RNA molecules is about 5,000 nt to about 9000 nt;
and the RNA recombination efficiency is about 10% to about 100%.

65. The composition of any one of embodiments 1 to 29, further comprising a one or more additional RNA molecules comprising one or more gRNAs specific for a one or more target nucleic acid molecules.

66. The composition of any one of embodiments 30 to 35, further comprising a one or more additional DNA molecules encoding one or more gRNAs specific for a one or more target nucleic acid molecules.

### Treatment of Duchenne Muscular Dystrophy (DMD)

Duchenne muscular dystrophy (DMD, MIM:310200) is a lethal hereditary disease characterized by progressive muscle weakness and degeneration. As the disease progresses, degenerating muscle fibres are replaced by fat and fibrotic tissue. DMD is rooted in deficiency of the gene dystrophin (MIM:300377). The dystrophin gene spans a region of 22 kbp, and is prone to mutations. Thus, DMD can in some cases sporadically manifest even in patients without a familial history of the disease-causing mutation. DMD is one of four conditions known as dystrophinopathies. The other three diseases that belong to this group are Becker Muscular dystrophy (BMD, a mild form of DMD); an intermediate clinical presentation between DMD and BMD; and DMD-associated dilated cardiomyopathy (heart-disease) with little or no clinical skeletal, or voluntary, muscle disease. Thus, in some examples a patient with DMD, BMD, an intermediate clinical presentation between DMD and BMD: or DMD-associated dilated cardiomyopathy (heart-disease) with little or no clinical skeletal, or voluntary, muscle disease, is treated with the disclosed systems and methods.

The disclosed methods and systems can be used to treat the monogenic cause of DMD by expressing one or more gRNAs which target a dystrophin mutation. Current methods of expressing dystrophin from a single AAV utilize shortened/truncated versions of dystrophin (micro-dystrophin and mini-dystrophin). Several of these truncated dystrophin delivery therapies are being tested in Phase I/I clinical trials (NCT03362502, NCT0428935, NCT03368742, NCT03375164). Although these truncated versions of dystrophin may ameliorate the worst consequences of dystrophin deficiency in DMD, they are not expected to have full functionality when compared to full-length dystrophin as the truncated versions are missing key domains in the rod and hinge region of the full-length protein. The disclosed methods and systems alleviate the size restriction of the transgenic payload of AAV by using “multiplexed” AAV combinations, because multiple AAV viruses can efficiently infect the same cell when introduced at high multiplicity of infection (MOI, i.e., high titer).

Thus, in some examples, a composition that includes two or more AAVs, each containing one of a set of disclosed synthetic molecules, is administered (e.g., i.v.) to a DMD subject in a therapeutically effective amount, such as a set that includes two, three, four or five different synthetic RNA molecules (each in a different AAV), which when recombined, result in a full-length nucleic acid editor protein coding sequence, wherein the composition further includes one or more gRNAs which target the dystrophin mutation.

### VII. Exemplary Embodiments-2

1. A system for expressing a target protein, comprising (a) a first synthetic nucleic acid molecule comprising a first promoter operably linked to a sequence encoding an RNA molecule, the RNA molecule comprising from 5′ to 3′: a coding sequence for an N-terminal portion of the target protein; a splice donor; and a first dimerization domain; and (b) a second synthetic nucleic acid molecule comprising a second promoter operably linked to a sequence encoding an RNA molecule, the RNA molecule comprising from 5′ to 3′: a second dimerization domain, wherein the second dimerization domain binds to the first dimerization domain; a branch point sequence; a polypyrimidine tract; a splice acceptor; and a coding sequence for a C-terminal portion of the target protein.

2. A system for expressing a target protein, comprising: (a) a first synthetic nucleic acid molecule comprising a first promoter operably linked to a sequence encoding an RNA molecule, the RNA molecule comprising from 5′ to 3′: a coding sequence for an N-terminal portion of the target protein; a splice donor; and a first dimerization domain; and (b) a second synthetic nucleic acid molecule comprising a second promoter operably linked to a sequence encoding an RNA molecule, the RNA molecule comprising from 5′ to 3′: a second dimerization domain, wherein the second dimerization domain binds to the first dimerization domain; a branch point sequence; a polypyrimidine tract; a splice acceptor; and a coding sequence for a middle portion of the target protein; a second splice donor; and a third dimerization domain; and (c) a third synthetic nucleic acid molecule comprising a third promoter operably linked to a sequence encoding an RNA molecule, the RNA molecule comprising from 5′ to 3′: a fourth dimerization domain, wherein the fourth dimerization domain binds to the third dimerization domain; a branch point sequence; a polypyrimidine tract; a splice acceptor; and a coding sequence for a C-terminal portion of the target protein.

3. A system for expressing a target protein, comprising (a) a first synthetic nucleic acid molecule comprising a first promoter operably linked to a sequence encoding an RNA molecule, the RNA molecule comprising from 5′ to 3′: a coding sequence for an N-terminal portion of the target protein, a splice donor, and a first dimerization domain; (b) a second synthetic nucleic acid molecule comprising a second promoter operably linked to a sequence encoding an RNA molecule, the RNA molecule comprising from 5′ to 3′: a second dimerization domain, wherein the second dimerization domain binds to the first dimerization domain; a branch point sequence; a polypyrimidine tract; a splice acceptor; and a coding sequence for a middle portion of the target protein; a second splice donor; and a third dimerization domain; and (c) a third synthetic nucleic acid molecule comprising a third promoter operably linked to a sequence encoding an RNA molecule, the RNA molecule comprising from 5′ to 3′: a fourth dimerization domain, wherein the fourth dimerization domain binds to the third dimerization domain; a branch point sequence; a polypyrimidine tract; a splice acceptor; and a coding sequence for a first middle portion of the target protein; a second splice donor; and a fifth dimerization domain; and (c) a fourth synthetic nucleic acid molecule comprising a fourth promoter operably linked to a sequence encoding an RNA molecule, the RNA molecule comprising from 5′ to 3′: a sixth dimerization domain, wherein the sixth dimerization domain binds to the fifth dimerization domain; a branch point sequence; a polypyrimidine tract; a splice acceptor, and a coding sequence for a C-terminal portion of the target protein.

4. The system of any one of embodiments 1 to 3, wherein each promoter is independently selected.

5. The system of any one of embodiments 1 to 4, wherein:
the first and second promoter are the same promoter;
the first and second promoter are different promoters;
the first, second, and third promoters are the same promoter;
the first, second, and third promoters are different promoters;
the first, second, third, and fourth promoters are the same promoter; or
the first, second, third and fourth promoters are different promoters.

6. The system of any one of embodiments 1 to 5, wherein each of the first, second, third, and fourth promoter is independently selected from: a constitutive promoter; a tissue-specific promoter; and a promoter endogenous to the target protein.

7. The system of any one of embodiments 1 to 6, wherein the first and second dimerization domains, third and fourth dimerization domains, and/or fifth and sixth dimerization domains, bind by direct binding, indirect binding, or a combination thereof.

8. The composition of claim 7, wherein direct binding or indirect binding comprises basepairing interactions, non-canonical base pairing interactions, non-base pairing interactions, or a combination thereof.

9. The composition of claim 7 or 8, wherein direct binding comprises base pairing interactions between kissing loops or hypodiverse regions.

10. The composition of claim 7 or 8, wherein direct binding comprises non-canonical basepairing interactions, non-canonical base pairing interactions, non-base pairing interactions, or a combination thereof, between aptamer regions.

11. The composition of claim 7 or 8, wherein indirect binding comprises basepairing interactions through a nucleic acid bridge.

12. The composition of claim 7 or 8, wherein indirect binding comprises non-base pairing interactions between an aptamer and an aptamer target, or between two aptamers.

13. The system of any one of embodiments 1 to 12, wherein the first, second, third, fourth, fifth and/or sixth dimerization domain does not comprise a cryptic splice acceptor.

14. The system of any one of embodiments 1 to 13, comprising at least one pair of directly binding or indirectly binding aptamer sequence dimerization domains.

15. The system of any one of embodiments 1 to 14, comprising at least one pair of kissing loop interaction dimerization domains.

16. The system of any one of embodiments 1 to 15, wherein the target protein is a protein associated with disease, or a therapeutic protein.

17. The system of embodiment 16, wherein the disease is a monogenic disease.

18. The system of embodiment 17, wherein the therapeutic protein is a toxin.

19. The system of any one of embodiments 16 to 18, wherein the disease and the target protein are one listed in Table 1.

20. The system of any one of embodiments 1 to 19, wherein the first, second, third, and/or fourth synthetic nucleic acid molecule further comprises a polyadenylation sequence at a 3′-end of the first, second, third, or fourth synthetic nucleic acid molecule.

21. The system of any one of embodiments 1 or 4 to 20, wherein the
the first synthetic nucleic acid molecule further comprises one or both of a downstream intronic splice enhancer (DISE) 3′ to the splice donor and 5′ to the first dimerization domain, an intronic splice enhancer (ISE) 3′ to the splice donor and 5′ to the first dimerization domain; and/or
the second synthetic nucleic acid molecule further comprises one or both of an ISE 3′ to the second dimerization domain and 5′ to the branch point sequence, and a DISE 3′ to the splice donor and 5′ to the dimerization domain;
and any combination thereof.

22. The system of any one of embodiments 2 or 4 to 20, wherein the
the first synthetic nucleic acid molecule further comprises a DISE 3′ to the first splice donor and 5′ to the first dimerization domain, an ISE 3′ to the first splice donor and 5′ to the first dimerization domain, or both a DISE and ISE;
the second synthetic nucleic acid molecule further comprises an ISE 3′ to the second dimerization domain and 5′ to the first branch point sequence, a DISE 3′ to the second splice donor and 5′ to the second dimerization domain, an ISE 3′ to the second splice donor and 5′ to the third dimerization domain, or combinations thereof; and/or
the third synthetic nucleic acid molecule further comprises an ISE 3′ to the fourth dimerization domain and 5′ to the second branch point sequence;
and any combination thereof.

23. The system of any one of embodiments 3 to 20, wherein
the first synthetic nucleic acid molecule further comprises a DISE 3′ to the first splice donor and 5′ to the first dimerization domain, an ISE 3′ to the first splice donor and 5′ to the first dimerization domain, or both a DISE and ISE;
the second synthetic nucleic acid molecule further comprises an ISE 3′ to the second dimerization domain and 5′ to the first branch point sequence, a DISE 3′ to the second splice donor and 5′ to the second dimerization domain, an ISE 3′ to the second splice donor and 5′ to the third dimerization domain, or combinations thereof;
the third synthetic nucleic acid molecule further comprises ISE 3′ to the fourth dimerization domain and 5′ to the second branch point sequence; and/or
the fourth synthetic nucleic acid molecule further comprises a ISE 3′ to the fifth dimerization domain and 5′ to the third branch point sequence, a DISE 3′ to the third splice donor and 5′ to the fifth dimerization domain, an ISE 3′ to the third splice donor and 5′ to the sixth dimerization domain, or combinations thereof;
and any combination thereof.

24. The system of any one of embodiments 1 to 23, wherein when the system is introduced into a cell the RNA molecules are produced and recombine in the proper order, resulting in a full-length coding sequence of the target protein.

25. The system of any one of embodiments 1 to 24, wherein each of the synthetic first, second, third and fourth nucleic acid molecules are part of a separate viral vector.

26. The system of embodiment 25, wherein the viral vector is AAV.

27. The system of any one of embodiments 1 to 26, wherein
the first and/or third synthetic nucleic acid molecule further comprises a self-cleaving RNA sequence or an RNA-cleaving enzyme target sequence positioned anywhere 3′ to the splice donor such that it cleaves off the 3′ located polyadenylated tail to decrease or suppress protein fragment expression from a non-recombined RNA molecule;
the second and/or fourth synthetic nucleic acid molecule further comprises a self-cleaving RNA sequence or an RNA-cleaving enzyme target sequence positioned anywhere 5′ to the branch point sequence such that it cleaves off the 5′ located RNA cap to decrease or suppress protein fragment expression from a non-recombined RNA molecule;
the second and/or fourth synthetic nucleic acid molecule further comprises a start codon anywhere 5′ to the branch point sequence that is shifted relative to the open reading frame 3′ of the splice acceptor to decrease or suppress translation of a target protein fragment from a non-recombined RNA molecule; the first and/or third synthetic nucleic acid molecule further comprises a micro RNA target site anywhere 3′ to the splice donor such that an un-joined RNA fragment undergoes micro RNA dependent degradation once outside the nucleus;
the second and/or fourth synthetic nucleic acid molecule further comprises a micro RNA target site anywhere 3′ to the coding sequence such that an un-joined RNA fragment undergoes micro RNA dependent degradation once outside the nucleus;
the first and/or third synthetic nucleic acid molecule further comprises a sequence encoding a degron protein degradation tag anywhere 3′ to the splice donor such that it is in frame with the target protein open reading frame 5′ of the splice donor site such that an un-joined protein fragment is tagged for degradation;
the second and/or fourth synthetic nucleic acid molecule further comprises a start codon and an in frame degron protein degradation tag anywhere 5′ to the branch point sequence such that it is in frame with the target protein open reading frame 3′ of the splice acceptor site such that an un-joined protein fragment is tagged for degradation;
or combinations thereof.

28. The system of any one of embodiments 1 to 27, wherein any one, two, three, or four synthetic nucleic acid molecules of the system each has a size independently selected from: about 2500 nt to about 5000 nt, 2,500 nt to about 2,750 nt, about 2,500 nt to about 3,000 nt, about 2,500 nt to about 3,250 nt, about 2,500 nt to about 3,500 nt, about 2,500 nt to about 3,750 nt, about 2,500 nt to about 4,000 nt, about 2,500 nt to about 4,250 nt, about 2,500 nt to about 4,500 nt, about 2,500 nt to about 4,750 nt, about 2,500 nt to about 5,000 nt, about 2,750 nt to about 3,000 nt, about 2,750 nt to about 3,250 nt, about 2,750 nt to about 3,500 nt, about 2,750 nt to about 3,750 nt, about 2,750 nt to about 4,000 nt, about 2,750 nt to about 4,250 nt, about 2,750 nt to about 4,500 nt, about 2,750 nt to about 4,750 nt, about 2,750 nt to about 5,000 nt, about 3,000 nt to about 3,250 nt, about 3,000 nt to about 3,500 nt, about 3,000 nt to about 3,750 nt, about 3,000 nt to about 4,000 nt, about 3,000 nt to about 4,250 nt, about 3,000 nt to about 4,500 nt, about 3,000 nt to about 4,750 nt, about 3,000 nt to about 5,000 nt, about 3,250 nt to about 3,500 nt, about 3,250 nt to about 3,750 nt, about 3,250 nt to about 4,000 nt, about 3,250 nt to about 4,250 nt, about 3,250 nt to about 4,500 nt, about 3,250 nt to about 4,750 nt, about 3,250 nt to about 5,000 nt, about 3,500 nt to about 3,750 nt, about 3,500 nt to about 4,000 nt, about 3,500 nt to about 4,250 nt, about 3,500 nt to about 4,500 nt, about 3,500 nt to about 4,750 nt, about 3,500 nt to about 5,000 nt, about 3,750 nt to about 4,000 nt, about 3,750 nt to about 4,250 nt, about 3,750 nt to about 4,500 nt, about 3,750 nt to about 4,750 nt, about 3,750 nt to about 5,000 nt, about 4,000 nt to about 4,250 nt, about 4,000 nt to about 4,500 nt, about 4,000 nt to about 4,750 nt, about 4,000 nt to about 5,000 nt, about 4,250 nt to about 4,500 nt, about 4,250 nt to about 4,750 nt, about 4,250 nt to about 5,000 nt, about 4,500 nt to about 4,750 nt, about 4,500 nt to about 5,000 nt, about 4,750 nt to about 5,000 nt, about 2,500 nt, about 2,750 nt, about 3,000 nt, about 3,250 nt, about 3,500 nt, about 3,750 nt, about 4,000 nt, about 4,250 nt, about 4,500 nt, about 4,750 nt, and about 5,000 nt.

29. The system of any one of embodiments 1 to 28, wherein the coding sequence for an N-terminal portion of the target protein, a middle portion of the target protein, or a C-terminal portion of the target protein encoded by a synthetic nucleic acid molecule of the system each has a size independently selected from: about 1000 nt to about 4000 nt, about 1,000 nt to about 1,500 nt, about 1,000 nt to about 2,000 nt, about 1,000 nt to about 2,500 nt, about 1,000 nt to about 3,000 nt, about 1,000 nt to about 3,500 nt, about 1,000 nt to about 4,000 nt, about 1,500 nt to about 2,000 nt, about 1,500 nt to about 2,500 nt, about 1,500 nt to about 3,000 nt, about 1,500 nt to about 3,500 nt, about 1,500 nt to about 4,000 nt, about 2,000 nt to about 2,500 nt, about 2,000 nt to about 3,000 nt, about 2,000 nt to about 3,500 nt, about 2,000 nt to about 4,000 nt, about 2,500 nt to about 3,000 nt, about 2,500 nt to about 3,500 nt, about 2,500 nt to about 4,000 nt, about 3,000 nt to about 3,500 nt, about 3,000 nt to about 4,000 nt, about 3,500 nt to about 4,000 nt, about 1,000 nt, about 1,500 nt, about 2,000 nt, about 2,500 nt, about 3,000 nt, about 3,500 nt, and about 4,000 nt.

30. The system of any one of embodiments 1 to 29, wherein any one, two, three, or four RNAs encoded by any of the one, two, three, or four synthetic nucleic acid molecules of the system, respectively, each has a size independently selected from: about 2500 to 4500 nt, about 2,500 nt to about 2,750 nt, about 2,500 nt to about 3,000 nt, about 2,500 nt to about 3,250 nt, about 2,500 nt to about 3,500 nt, about 2,500 nt to about 3,750 nt, about 2,500 nt to about 4,000 nt, about 2,500 nt to about 4,250 nt, about 2,500 nt to about 4,500 nt, about 2,750 nt to about 3,000 nt, about 2,750 nt to about 3,250 nt, about 2,750 nt to about 3,500 nt, about 2,750 nt to about 3,750 nt, about 2,750 nt to about 4,000 nt, about 2,750 nt to about 4,250 nt, about 2,750 nt to about 4,500 nt, about 3,000 nt to about 3,250 nt, about 3,000 nt to about 3,500 nt, about 3,000 nt to about 3,750 nt, about 3,000 nt to about 4,000 nt, about 3,000 nt to about 4,250 nt, about 3,000 nt to about 4,500 nt, about 3,250 nt to about 3,500 nt, about 3,250 nt to about 3,750 nt, about 3,250 nt to about 4,000 nt, about 3,250 nt to about 4,250 nt, about 3,250 nt to about 4,500 nt, about 3,500 nt to about 3,750 nt, about 3,500 nt to about 4,000 nt, about 3,500 nt to about 4,250 nt, about 3,500 nt to about 4,500 nt, about 3,750 nt to about 4,000 nt, about 3,750 nt to about 4,250 nt, about 3,750 nt to about 4,500 nt, about 4,000 nt to about 4,250 nt, about 4,000 nt to about 4,500 nt, about 4,250 nt to about 4,500 nt, about 2,500 nt, about 2,750 nt, about 3,000 nt, about 3,250 nt, about 3,500 nt, about 3,750 nt, about 4,000 nt, about 4,250 nt, and about 4,500 nt. 31. The system of any one of embodiments 1 and 4 to 30, wherein:
the synthetic nucleic acid molecules have a total size selected from about 5000 nt to about 10,000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,000 nt to about 8,500 nt, about 5,000 nt to about 9,000 nt, about 5,000 nt to about 9,500 nt, about 5,000 nt to about 10,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 5,500 nt to about 8,500 nt, about 5,500 nt to about 9,000 nt, about 5,500 nt to about 9,500 nt, about 5,500 nt to about 10,000 nt, about 6,000 nt to about 6,500 nt, about 6,000 nt to about 7,000 nt, about 6,000 nt to about 7,500 nt, about 6,000 nt to about 8,000 nt, about 6,000 nt to about 8,500 nt, about 6,000 nt to about 9,000 nt, about 6,000 nt to about 9,500 nt, about 6,000 nt to about 10,000 nt, about 6,500 nt to about 7,000 nt, about 6,500 nt to about 7,500 nt, about 6,500 nt to about 8,000 nt, about 6,500 nt to about 8,500 nt, about 6,500 nt to about 9,000 nt, about 6,500 nt to about 9,500 nt, about 6,500 nt to about 10,000 nt, about 7,000 nt to about 7,500 nt, about 7,000 nt to about 8,000 nt, about 7,000 nt to about 8,500 nt, about 7,000 nt to about 9,000 nt, about 7,000 nt to about 9,500 nt, about 7,000 nt to about 10,000 nt, about 7,500 nt to about 8,000 nt, about 7,500 nt to about 8,500 nt, about 7,500 nt to about 9,000 nt, about 7,500 nt to about 9,500 nt, about 7,500 nt to about 10,000 nt, about 8,000 nt to about 8,500 nt, about 8,000 nt to about 9,000 nt, about 8,000 nt to about 9,500 nt, about 8,000 nt to about 10,000 nt, about 8,500 nt to about 9,000 nt, about 8,500 nt to about 9,500 nt, about 8,500 nt to about 10,000 nt, about 9,000 nt to about 9,500 nt, about 9,000 nt to about 10,000 nt, about 9,500 nt to about 10,000 nt, about 5,000 nt, about 5,500 nt, about 6,000 nt, about 6,500 nt, about 7,000 nt, about 7,500 nt, about 8,000 nt, about 8,500 nt, about 9,000 nt, about 9,500 nt, and about 10,000 nt;
the total target protein coding sequence is selected from about 2000 nt to about 8000 nt, about 2,000 nt to about 3,000 nt, about 2,000 nt to about 3,500 nt, about 2,000 nt to about 4,000 nt, about 2,000 nt to about 4,500 nt, about 2,000 nt to about 5,000 nt, about 2,000 nt to about 5,500 nt, about 2,000 nt to about 6,000 nt, about 2,000 nt to about 6,500 nt, about 2,000 nt to about 7,000 nt, about 2,000 nt to about 7,500 nt, about 2,000 nt to about 8,000 nt, about 3,000 nt to about 3,500 nt, about 3,000 nt to about 4,000 nt, about 3,000 nt to about 4,500 nt, about 3,000 nt to about 5,000 nt, about 3,000 nt to about 5,500 nt, about 3,000 nt to about 6,000 nt, about 3,000 nt to about 6,500 nt, about 3,000 nt to about 7,000 nt, about 3,000 nt to about 7,500 nt, about 3,000 nt to about 8,000 nt, about 3,500 nt to about 4,000 nt, about 3,500 nt to about 4,500 nt, about 3,500 nt to about 5,000 nt, about 3,500 nt to about 5,500 nt, about 3,500 nt to about 6,000 nt, about 3,500 nt to about 6,500 nt, about 3,500 nt to about 7,000 nt, about 3,500 nt to about 7,500 nt, about 3,500 nt to about 8,000 nt, about 4,000 nt to about 4,500 nt, about 4,000 nt to about 5,000 nt, about 4,000 nt to about 5,500 nt, about 4,000 nt to about 6,000 nt, about 4,000 nt to about 6,500 nt, about 4,000 nt to about 7,000 nt, about 4,000 nt to about 7,500 nt, about 4,000 nt to about 8,000 nt, about 4,500 nt to about 5,000 nt, about 4,500 nt to about 5,500 nt, about 4,500 nt to about 6,000 nt, about 4,500 nt to about 6,500 nt, about 4,500 nt to about 7,000 nt, about 4,500 nt to about 7,500 nt, about 4,500 nt to about 8,000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 6,000 nt to about 6,500 nt, about 6,000 nt to about 7,000 nt, about 6,000 nt to about 7,500 nt, about 6,000 nt to about 8,000 nt, about 6,500 nt to about 7,000 nt, about 6,500 nt to about 7,500 nt, about 6,500 nt to about 8,000 nt, about 7,000 nt to about 7,500 nt, about 7,000 nt to about 8,000 nt, or about 7,500 nt to about 8,000 nt, the total target protein coding sequence is about 2,000 nt, about 3,000 nt, about 3,500 nt, about 4,000 nt, about 4,500 nt, about 5,000 nt, about 5,500 nt, about 6,000 nt, about 6,500 nt, about 7,000 nt, about 7,500 nt, and about 8,000 nt; and/or
the RNA encoded by the two synthetic nucleic acid molecules has a total size selected from about 5,000 nt to about 9000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,000 nt to about 8,500 nt, about 5,000 nt to about 9,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 5,500 nt to about 8,500 nt, about 5,500 nt to about 9,000 nt, about 6,000 nt to about 6,500 nt, about 6,000 nt to about 7,000 nt, about 6,000 nt to about 7,500 nt, about 6,000 nt to about 8,000 nt, about 6,000 nt to about 8,500 nt, about 6,000 nt to about 9,000 nt, about 6,500 nt to about 7,000 nt, about 6,500 nt to about 7,500 nt, about 6,500 nt to about 8,000 nt, about 6,500 nt to about 8,500 nt, about 6,500 nt to about 9,000 nt, about 7,000 nt to about 7,500 nt, about 7,000 nt to about 8,000 nt, about 7,000 nt to about 8,500 nt, about 7,000 nt to about 9,000 nt, about 7,500 nt to about 8,000 nt, about 7,500 nt to about 8,500 nt, about 7,500 nt to about 9,000 nt, about 8,000 nt to about 8,500 nt, about 8,000 nt to about 9,000 nt, or about 8,500 nt to about 9,000 nt, the RNA encoded by the two synthetic nucleic acid molecules has a total size of about 5,000 nt, about 5,500 nt, about 6,000 nt, about 6,500 nt, about 7,000 nt, about 7,500 nt, about 8,000 nt, about 8,500 nt, and about 9,000 nt.

32. The system of any one of embodiments 2 and 4 to 30, wherein:
the synthetic nucleic acid molecules have a total size selected from about 7500 nt to about 15,000 nt, about 7,500 nt to about 8,500 nt, about 7,500 nt to about 9,500 nt, about 7,500 nt to about 10,000 nt, about 7,500 nt to about 10,500 nt, about 7,500 nt to about 11,000 nt, about 7,500 nt to about 11,500 nt, about 7,500 nt to about 12,000 nt, about 7,500 nt to about 12,500 nt, about 7,500 nt to about 13,000 nt, about 7,500 nt to about 14,000 nt, about 7,500 nt to about 15,000 nt, about 8,500 nt to about 9,500 nt, about 8,500 nt to about 10,000 nt, about 8,500 nt to about 10,500 nt, about 8,500 nt to about 11,000 nt, about 8,500 nt to about 11,500 nt, about 8,500 nt to about 12,000 nt, about 8,500 nt to about 12,500 nt, about 8,500 nt to about 13,000 nt, about 8,500 nt to about 14,000 nt, about 8,500 nt to about 15,000 nt, about 9,500 nt to about 10,000 nt, about 9,500 nt to about 10,500 nt, about 9,500 nt to about 11,000 nt, about 9,500 nt to about 11,500 nt, about 9,500 nt to about 12,000 nt, about 9,500 nt to about 12,500 nt, about 9,500 nt to about 13,000 nt, about 9,500 nt to about 14,000 nt, about 9,500 nt to about 15,000 nt, about 10,000 nt to about 10,500 nt, about 10,000 nt to about 11,000 nt, about 10,000 nt to about 11,500 nt, about 10,000 nt to about 12,000 nt, about 10,000 nt to about 12,500 nt, about 10,000 nt to about 13,000 nt, about 10,000 nt to about 14,000 nt, about 10,000 nt to about 15,000 nt, about 10,500 nt to about 11,000 nt, about 10,500 nt to about 11,500 nt, about 10,500 nt to about 12,000 nt, about 10,500 nt to about 12,500 nt, about 10,500 nt to about 13,000 nt, about 10,500 nt to about 14,000 nt, about 10,500 nt to about 15,000 nt, about 11,000 nt to about 11,500 nt, about 11,000 nt to about 12,000 nt, about 11,000 nt to about 12,500 nt, about 11,000 nt to about 13,000 nt, about 11,000 nt to about 14,000 nt, about 11,000 nt to about 15,000 nt, about 11,500 nt to about 12,000 nt, about 11,500 nt to about 12,500 nt, about 11,500 nt to about 13,000 nt, about 11,500 nt to about 14,000 nt, about 11,500 nt to about 15,000 nt, about 12,000 nt to about 12,500 nt, about 12,000 nt to about 13,000 nt, about 12,000 nt to about 14,000 nt, about 12,000 nt to about 15,000 nt, about 12,500 nt to about 13,000 nt, about 12,500 nt to about 14,000 nt, about 12,500 nt to about 15,000 nt, about 13,000 nt to about 14,000 nt, about 13,000 nt to about 15,000 nt, or about 14,000 nt to about 15,000 nt, the synthetic nucleic acid molecules have a total size of about 7,500 nt, about 8,500 nt, about 9,500 nt, about 10,000 nt, about 10,500 nt, about 11,000 nt, about 11,500 nt, about 12,000 nt, about 12,500 nt, about 13,000 nt, about 14,000 nt, and about 15,000 nt;
the total target protein coding sequence is selected from about 3000 nt to about 12,000 nt, about 3,000 nt to about 4,000 nt, about 3,000 nt to about 5,000 nt, about 3,000 nt to about 6,000 nt, about 3,000 nt to about 7,000 nt, about 3,000 nt to about 7,500 nt, about 3,000 nt to about 8,000 nt, about 3,000 nt to about 8,500 nt, about 3,000 nt to about 9,000 nt, about 3,000 nt to about 1,000 nt, about 3,000 nt to about 11,000 nt, about 3,000 nt to about 12,000 nt, about 4,000 nt to about 5,000 nt, about 4,000 nt to about 6,000 nt, about 4,000 nt to about 7,000 nt, about 4,000 nt to about 7,500 nt, about 4,000 nt to about 8,000 nt, about 4,000 nt to about 8,500 nt, about 4,000 nt to about 9,000 nt, about 4,000 nt to about 1,000 nt, about 4,000 nt to about 11,000 nt, about 4,000 nt to about 12,000 nt, about 5,000 nt to about 6,000 nt, about 5,000 nt to about 7,000 nt, about 5,000 nt to about 7,500 nt, about 5,000 nt to about 8,000 nt, about 5,000 nt to about 8,500 nt, about 5,000 nt to about 9,000 nt, about 5,000 nt to about 1,000 nt, about 5,000 nt to about 11,000 nt, about 5,000 nt to about 12,000 nt, about 6,000 nt to about 7,000 nt, about 6,000 nt to about 7,500 nt, about 6,000 nt to about 8,000 nt, about 6,000 nt to about 8,500 nt, about 6,000 nt to about 9,000 nt, about 6,000 nt to about 1,000 nt, about 6,000 nt to about 11,000 nt, about 6,000 nt to about 12,000 nt, about 7,000 nt to about 7,500 nt, about 7,000 nt to about 8,000 nt, about 7,000 nt to about 8,500 nt, about 7,000 nt to about 9,000 nt, about 7,000 nt to about 1,000 nt, about 7,000 nt to about 11,000 nt, about 7,000 nt to about 12,000 nt, about 7,500 nt to about 8,000 nt, about 7,500 nt to about 8,500 nt, about 7,500 nt to about 9,000 nt, about 7,500 nt to about 1,000 nt, about 7,500 nt to about 11,000 nt, about 7,500 nt to about 12,000 nt, about 8,000 nt to about 8,500 nt, about 8,000 nt to about 9,000 nt, about 8,000 nt to about 1,000 nt, about 8,000 nt to about 11,000 nt, about 8,000 nt to about 12,000 nt, about 8,500 nt to about 9,000 nt, about 8,500 nt to about 1,000 nt, about 8,500 nt to about 11,000 nt, about 8,500 nt to about 12,000 nt, about 9,000 nt to about 1,000 nt, about 9,000 nt to about 11,000 nt, about 9,000 nt to about 12,000 nt, about 1,000 nt to about 11,000 nt, about 1,000 nt to about 12,000 nt, or about 11,000 nt to about 12,000 nt, the total target protein coding sequence is about 3,000 nt, about 4,000 nt, about 5,000 nt, about 6,000 nt, about 7,000 nt, about 7,500 nt, about 8,000 nt, about 8,500 nt, about 9,000 nt, about 1,000 nt, about 11,000 nt, and about 12,000 nt; and/or
the RNA encoded by the three synthetic nucleic acid molecules has a total size selected from about 7500 nt to about 13,500 nt, about 7,500 nt to about 8,500 nt, about 7,500 nt to about 9,000 nt, about 7,500 nt to about 9,500 nt, about 7,500 nt to about 10,000 nt, about 7,500 nt to about 10,500 nt, about 7,500 nt to about 11,000 nt, about 7,500 nt to about 11,500 nt, about 7,500 nt to about 12,000 nt, about 7,500 nt to about 12,500 nt, about 7,500 nt to about 13,000 nt, about 7,500 nt to about 13,500 nt, about 8,500 nt to about 9,000 nt, about 8,500 nt to about 9,500 nt, about 8,500 nt to about 10,000 nt, about 8,500 nt to about 10,500 nt, about 8,500 nt to about 11,000 nt, about 8,500 nt to about 11,500 nt, about 8,500 nt to about 12,000 nt, about 8,500 nt to about 12,500 nt, about 8,500 nt to about 13,000 nt, about 8,500 nt to about 13,500 nt, about 9,000 nt to about 9,500 nt, about 9,000 nt to about 10,000 nt, about 9,000 nt to about 10,500 nt, about 9,000 nt to about 11,000 nt, about 9,000 nt to about 11,500 nt, about 9,000 nt to about 12,000 nt, about 9,000 nt to about 12,500 nt, about 9,000 nt to about 13,000 nt, about 9,000 nt to about 13,500 nt, about 9,500 nt to about 10,000 nt, about 9,500 nt to about 10,500 nt, about 9,500 nt to about 11,000 nt, about 9,500 nt to about 11,500 nt, about 9,500 nt to about 12,000 nt, about 9,500 nt to about 12,500 nt, about 9,500 nt to about 13,000 nt, about 9,500 nt to about 13,500 nt, about 10,000 nt to about 10,500 nt, about 10,000 nt to about 11,000 nt, about 10,000 nt to about 11,500 nt, about 10,000 nt to about 12,000 nt, about 10,000 nt to about 12,500 nt, about 10,000 nt to about 13,000 nt, about 10,000 nt to about 13,500 nt, about 10,500 nt to about 11,000 nt, about 10,500 nt to about 11,500 nt, about 10,500 nt to about 12,000 nt, about 10,500 nt to about 12,500 nt, about 10,500 nt to about 13,000 nt, about 10,500 nt to about 13,500 nt, about 11,000 nt to about 11,500 nt, about 11,000 nt to about 12,000 nt, about 11,000 nt to about 12,500 nt, about 11,000 nt to about 13,000 nt, about 11,000 nt to about 13,500 nt, about 11,500 nt to about 12,000 nt, about 11,500 nt to about 12,500 nt, about 11,500 nt to about 13,000 nt, about 11,500 nt to about 13,500 nt, about 12,000 nt to about 12,500 nt, about 12,000 nt to about 13,000 nt, about 12,000 nt to about 13,500 nt, about 12,500 nt to about 13,000 nt, about 12,500 nt to about 13,500 nt, or about 13,000 nt to about 13,500 nt, the RNA encoded by the two synthetic nucleic acid molecules has a total size of about 7,500 nt, about 8,500 nt, about 9,000 nt, about 9,500 nt, about 10,000 nt, about 10,500 nt, about 11,000 nt, about 11,500 nt, about 12,000 nt, about 12,500 nt, about 13,000 nt, and about 13,500 nt.

33. The system of any one of embodiments 3 and 4 to 30, wherein:
the synthetic nucleic acid molecules have a total size selected from about 10,000 nt to about 20,000 nt, about 10,000 nt to about 11,000 nt, about 10,000 nt to about 12,000 nt, about 10,000 nt to about 13,000 nt, about 10,000 nt to about 14,000 nt, about 10,000 nt to about 15,000 nt, about 10,000 nt to about 16,000 nt, about 10,000 nt to about 17,000 nt, about 10,000 nt to about 18,000 nt, about 10,000 nt to about 19,000 nt, about 10,000 nt to about 20,000 nt, about 11,000 nt to about 12,000 nt, about 11,000 nt to about 13,000 nt, about 11,000 nt to about 14,000 nt, about 11,000 nt to about 15,000 nt, about 11,000 nt to about 16,000 nt, about 11,000 nt to about 17,000 nt, about 11,000 nt to about 18,000 nt, about 11,000 nt to about 19,000 nt, about 11,000 nt to about 20,000 nt, about 12,000 nt to about 13,000 nt, about 12,000 nt to about 14,000 nt, about 12,000 nt to about 15,000 nt, about 12,000 nt to about 16,000 nt, about 12,000 nt to about 17,000 nt, about 12,000 nt to about 18,000 nt, about 12,000 nt to about 19,000 nt, about 12,000 nt to about 20,000 nt, about 13,000 nt to about 14,000 nt, about 13,000 nt to about 15,000 nt, about 13,000 nt to about 16,000 nt, about 13,000 nt to about 17,000 nt, about 13,000 nt to about 18,000 nt, about 13,000 nt to about 19,000 nt, about 13,000 nt to about 20,000 nt, about 14,000 nt to about 15,000 nt, about 14,000 nt to about 16,000 nt, about 14,000 nt to about 17,000 nt, about 14,000 nt to about 18,000 nt, about 14,000 nt to about 19,000 nt, about 14,000 nt to about 20,000 nt, about 15,000 nt to about 16,000 nt, about 15,000 nt to about 17,000 nt, about 15,000 nt to about 18,000 nt, about 15,000 nt to about 19,000 nt, about 15,000 nt to about 20,000 nt, about 16,000 nt to about 17,000 nt, about 16,000 nt to about 18,000 nt, about 16,000 nt to about 19,000 nt, about 16,000 nt to about 20,000 nt, about 17,000 nt to about 18,000 nt, about 17,000 nt to about 19,000 nt, about 17,000 nt to about 20,000 nt, about 18,000 nt to about 19,000 nt, about 18,000 nt to about 20,000 nt, or about 19,000 nt to about 20,000 nt, the synthetic nucleic acid molecules have a total size of about 10,000 nt, about 11,000 nt, about 12,000 nt, about 13,000 nt, about 14,000 nt, about 15,000 nt, about 16,000 nt, about 17,000 nt, about 18,000 nt, about 19,000 nt, and about 20,000 nt;
the total target protein coding sequence is selected from about 4000 nt to about 16,000 nt, about 5,000 nt to about 6,000 nt, about 5,000 nt to about 7,000 nt, about 5,000 nt to about 8,000 nt, about 5,000 nt to about 9,000 nt, about 5,000 nt to about 10,000 nt, about 5,000 nt to about 11,000 nt, about 5,000 nt to about 12,000 nt, about 5,000 nt to about 13,000 nt, about 5,000 nt to about 14,000 nt, about 5,000 nt to about 15,000 nt, about 5,000 nt to about 16,000 nt, about 6,000 nt to about 7,000 nt, about 6,000 nt to about 8,000 nt, about 6,000 nt to about 9,000 nt, about 6,000 nt to about 10,000 nt, about 6,000 nt to about 11,000 nt, about 6,000 nt to about 12,000 nt, about 6,000 nt to about 13,000 nt, about 6,000 nt to about 14,000 nt, about 6,000 nt to about 15,000 nt, about 6,000 nt to about 16,000 nt, about 7,000 nt to about 8,000 nt, about 7,000 nt to about 9,000 nt, about 7,000 nt to about 10,000 nt, about 7,000 nt to about 11,000 nt, about 7,000 nt to about 12,000 nt, about 7,000 nt to about 13,000 nt, about 7,000 nt to about 14,000 nt, about 7,000 nt to about 15,000 nt, about 7,000 nt to about 16,000 nt, about 8,000 nt to about 9,000 nt, about 8,000 nt to about 10,000 nt, about 8,000 nt to about 11,000 nt, about 8,000 nt to about 12,000 nt, about 8,000 nt to about 13,000 nt, about 8,000 nt to about 14,000 nt, about 8,000 nt to about 15,000 nt, about 8,000 nt to about 16,000 nt, about 9,000 nt to about 10,000 nt, about 9,000 nt to about 11,000 nt, about 9,000 nt to about 12,000 nt, about 9,000 nt to about 13,000 nt, about 9,000 nt to about 14,000 nt, about 9,000 nt to about 15,000 nt, about 9,000 nt to about 16,000 nt, about 10,000 nt to about 11,000 nt, about 10,000 nt to about 12,000 nt, about 10,000 nt to about 13,000 nt, about 10,000 nt to about 14,000 nt, about 10,000 nt to about 15,000 nt, about 10,000 nt to about 16,000 nt, about 11,000 nt to about 12,000 nt, about 11,000 nt to about 13,000 nt, about 11,000 nt to about 14,000 nt, about 11,000 nt to about 15,000 nt, about 11,000 nt to about 16,000 nt, about 12,000 nt to about 13,000 nt, about 12,000 nt to about 14,000 nt, about 12,000 nt to about 15,000 nt, about 12,000 nt to about 16,000 nt, about 13,000 nt to about 14,000 nt, about 13,000 nt to about 15,000 nt, about 13,000 nt to about 16,000 nt, about 14,000 nt to about 15,000 nt, about 14,000 nt to about 16,000 nt, or about 15,000 nt to about 16,000 nt, the total target protein coding sequence is about 5,000 nt, about 6,000 nt, about 7,000 nt, about 8,000 nt, about 9,000 nt, about 10,000 nt, about 11,000 nt, about 12,000 nt, about 13,000 nt, about 14,000 nt, about 15,000 nt, or about 16,000 nt, the total target protein coding sequence is at least about 5,000 nt, about 6,000 nt, about 7,000 nt, about 8,000 nt, about 9,000 nt, about 10,000 nt, about 11,000 nt, about 12,000 nt, about 13,000 nt, about 14,000 nt, and about 15,000 nt; and/or
the RNA encoded by the two synthetic nucleic acid molecules has a total size selected from about 10,000 nt to about 18,000 nt, about 10,000 nt to about 11,000 nt, about 10,000 nt to about 12,000 nt, about 10,000 nt to about 13,000 nt, about 10,000 nt to about 14,000 nt, about 10,000 nt to about 15,000 nt, about 10,000 nt to about 16,000 nt, about 10,000 nt to about 17,000 nt, about 10,000 nt to about 18,000 nt, about 11,000 nt to about 12,000 nt, about 11,000 nt to about 13,000 nt, about 11,000 nt to about 14,000 nt, about 11,000 nt to about 15,000 nt, about 11,000 nt to about 16,000 nt, about 11,000 nt to about 17,000 nt, about 11,000 nt to about 18,000 nt, about 12,000 nt to about 13,000 nt, about 12,000 nt to about 14,000 nt, about 12,000 nt to about 15,000 nt, about 12,000 nt to about 16,000 nt, about 12,000 nt to about 17,000 nt, about 12,000 nt to about 18,000 nt, about 13,000 nt to about 14,000 nt, about 13,000 nt to about 15,000 nt, about 13,000 nt to about 16,000 nt, about 13,000 nt to about 17,000 nt, about 13,000 nt to about 18,000 nt, about 14,000 nt to about 15,000 nt, about 14,000 nt to about 16,000 nt, about 14,000 nt to about 17,000 nt, about 14,000 nt to about 18,000 nt, about 15,000 nt to about 16,000 nt, about 15,000 nt to about 17,000 nt, about 15,000 nt to about 18,000 nt, about 16,000 nt to about 17,000 nt, about 16,000 nt to about 18,000 nt, or about 17,000 nt to about 18,000 nt, the RNA encoded by the two synthetic nucleic acid molecules has a total size of about 10,000 nt, about 11,000 nt, about 12,000 nt, about 13,000 nt, about 14,000 nt, about 15,000 nt, about 16,000 nt, about 17,000 nt, and about 18,000 nt.

34. The system of any one of embodiments 1 to 33, wherein the RNA recombination efficiency is about 10% to about 95%, about 10% to about 20%, about 10% to about 30%, about 10% to about 35%, about 10% to about 40%, about 10% to about 45%, about 10% to about 50%, about 10% to about 55%, about 10% to about 60%, about 10% to about 70%, about 10% to about 80%, about 10% to about 90%, about 20% to about 30%, about 20% to about 35%, about 20% to about 40%, about 20% to about 45%, about 20% to about 50%, about 20% to about 55%, about 20% to about 60%, about 20% to about 70%, about 20% to about 80%, about 20% to about 90%, about 30% to about 35%, about 30% to about 40%, about 30% to about 45%, about 30% to about 50%, about 30% to about 55%, about 30% to about 60%, about 30% to about 70%, about 30% to about 80%, about 30% to about 90%, about 35% to about 40%, about 35% to about 45%, about 35% to about 50%, about 35% to about 55%, about 35% to about 60%, about 35% to about 70%, about 35% to about 80%, about 35% to about 90%, about 40% to about 45%, about 40% to about 50%, about 40% to about 55%, about 40% to about 60%, about 40% to about 70%, about 40% to about 80%, about 40% to about 90%, about 45% to about 50%, about 45% to about 55%, about 45% to about 60%, about 45% to about 70%, about 45% to about 80%, about 45% to about 90%, about 50% to about 55%, about 50% to about 60%, about 50% to about 70%, about 50% to about 80%, about 50% to about 90%, about 55% to about 60%, about 55% to about 70%, about 55% to about 80%, about 55% to about 90%, about 60% to about 70%, about 60% to about 80%, about 60% to about 90%, about 70% to about 80%, about 70% to about 90%, about 80% to about 90%, about 10%, about 20%, about 30%, about 35%, about 40%, about 45%, about 50%, about 55%, about 60%, about 70%, about 80%, or about 90%, or about 95%.

35. The system of any one of embodiments 1 to 34, wherein the first dimerization domain and the second dimerization domain, the third dimerization domain and the fourth dimerization domain, and/or the fifth dimerization domain and the sixth dimerization domain, are each no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150, 200, 250, 300, 400, or 500 nt; and the system has a recombination efficiency of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

36. The system of any one of embodiments 1 to 35, wherein each dimerization domain is no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150, 200, 250, 300, 400, or 500 nt; and the system has a recombination efficiency of at least 20%, at least 30%, at least 40%, at least 50%, at least 60%, at least 70%, at least 75%, at least 80%, or at least 90%.

37. A composition comprising the system of any one of embodiments 1 to 36.

38. A composition comprising an RNA molecule as described in any one of embodiments 1 to 37.

39. A composition comprising one, two, three, or four RNA molecules as described in any one of embodiments 1 to 37.

40. The composition of any one of embodiments 37 to 39, wherein the composition comprises first, second, third and optionally fourth synthetic nucleic acid or RNA molecules, each encoding at least a portion of dystrophin, factor 8, ABCA4, or MYO7A.

41. An RNA molecule as described in any one of embodiments 1 to 36.

42. A kit comprising the system of any one of embodiments 1 to 41, or composition of any one of embodiments 37 to 40, wherein any of the synthetic first, second, third and fourth nucleic acid molecules can be in separate containers, and optionally further comprising a buffer such as a pharmaceutically acceptable carrier.

43. A method of expressing a target protein in a cell, comprising:
introducing the system of any one of embodiments 1 to 36, or composition of any one of embodiments 35 to 37, into a cell, and expressing the synthetic first and second, first, second, and third, or first, second, third and fourth RNA molecules in the cell, wherein the target protein is produced in the cell.

44. The method of embodiment 43, wherein the cell is in a subject, and introducing comprises administering a therapeutically effective amount the system to the subject.

45. The method of embodiment 44, wherein the method treats a genetic disease caused by a mutation in a gene encoding the target protein in the subject, wherein the method results in expression of functional target protein in the subject.

46. The method of embodiment 45, wherein
the genetic disease is Duchenne muscular dystrophy and the target protein is dystrophin;
the genetic disease is Hemophilia A and the target protein is F8;
the genetic disease is Stargardt disease and the target protein is ABCA4; or
the genetic disease is Usher syndrome and the target protein is MYO7A.

47. A nucleic acid molecule comprising at least 80%, at least 85%, at least 90%, at least 95%, at least 98%, at least 99%, or 100% sequence identity to a synthetic intron provided in any one of SEQ ID NOS: 1, 2, 3, 4, 5, 6, 7, 8, 9, 10, 11, 12, 13, 14, 15, 16, 17, 18, 19, 20, 21, 22, 23, 24, 25, 145, 146, 147, 148, 155, 156, 157, 158, 159, 160, 161, 162, 163, 164, 165, and 166.

48. The nucleic acid molecule of embodiment 47, wherein the synthetic intron is nt 3703 to 3975 of SEQ ID NO: 20, nt 1 to 228 of SEQ ID NO: 21, nt 3703 to 3975 of SEQ ID NO: 22, nt 1 to 225 of SEQ ID NO: 23, nt 3560 to 3828 of SEQ ID NO: 24, or nt 1-225 of SEQ ID NO: 25.

49. The synthetic nucleic acid molecule of embodiment 47 or 48, further comprising a portion of a protein coding sequence.

50. The synthetic nucleic acid molecule of embodiment 49, wherein the portion of the protein coding sequence comprises an N-terminal half, an N-terminal third, a middle portion, a C-terminal half, or a C-terminal third of the protein coding sequence.

51. A system of any one of embodiments 1 to 36, or composition of any one of embodiments 37 to 40, wherein at least one synthetic nucleic acid molecule comprises a synthetic intron comprising a nucleic acid molecule as set forth in any one of embodiments 47 to 50.

52. The composition, system, method, or kit of any preceding embodiment, wherein the synthetic nucleic acid is DNA that is produced by transcription of an RNA virus genome by reverse transcriptase.

### VIII. Exemplary Embodiments-3

1. A composition for expressing a target protein comprising (a) a first RNA molecule, the RNA molecule comprising from 5′ to 3′: (i) a coding sequence for an N-terminal portion of the target protein; (ii) a splice donor; and (iii) a first dimerization domain; and (b) a second RNA molecule, the RNA molecule comprising from 5′ to 3′: (i) a second dimerization domain, wherein the second dimerization domain binds to the first dimerization domain; (ii) a branch point sequence; (iii) a polypyrimidine tract; (iv) a splice acceptor; and (v) a coding sequence for a C-terminal portion of the target protein.

2. A composition for expressing a target protein comprising: (a) a first RNA molecule, the RNA molecule comprising from 5′ to 3′: (i) a coding sequence for an N-terminal portion of the target protein; (ii) a splice donor; and (iii) a first dimerization domain; and (b) a second RNA molecule, the RNA molecule comprising from 5′ to 3′: (i) a second dimerization domain, wherein the second dimerization domain binds to the first dimerization domain; (ii) a branch point sequence; (iii) a polypyrimidine tract; (iv) a splice acceptor; (v) a coding sequence for a middle portion of the target protein; (vi) a second splice donor; and (vii) a third dimerization domain; and (c) a third RNA molecule, the RNA molecule comprising from 5′ to 3′: (i) a fourth dimerization domain, wherein the fourth dimerization domain binds to the third dimerization domain; (ii) a branch point sequence; (iii) a polypyrimidine tract; (iv) a splice acceptor; and (v) a coding sequence for a C-terminal portion of the target protein. 3. A composition for expressing a target protein comprising (a) a first RNA molecule, the RNA molecule comprising from 5′ to 3′: (i) a coding sequence for an N-terminal portion of the target protein, (ii) a splice donor; and (iii) a first dimerization domain; (b) a second RNA molecule, the RNA molecule comprising from 5′ to 3′: (i) a second dimerization domain, wherein the second dimerization domain binds to the first dimerization domain; (ii) a branch point sequence; (iii) a polypyrimidine tract; (iv) a splice acceptor; (v) a coding sequence for a middle portion of the target protein; (vi) a second splice donor; and (vii) a third dimerization domain; and (c) a third RNA molecule, the RNA molecule comprising from 5′ to 3′: (i) a fourth dimerization domain, wherein the fourth dimerization domain binds to the third dimerization domain; (ii) a branch point sequence; (iii) a polypyrimidine tract; (iv) a splice acceptor; (v) a coding sequence for a first middle portion of the target protein; (vi) a second splice donor; and (vii) a fifth dimerization domain; and (d) a fourth RNA molecule, the RNA molecule comprising from 5′ to 3′: (i) a sixth dimerization domain, wherein the sixth dimerization domain binds to the fifth dimerization domain; (ii) a branch point sequence; (iii) a polypyrimidine tract; (iv) a splice acceptor; and (v) a coding sequence for a C-terminal portion of the target protein.

4. The composition of any one of embodiments 1 to 3, wherein the first and second dimerization domains, third and fourth dimerization domains, and/or fifth and sixth dimerization domains, bind by direct binding, indirect binding, or a combination thereof.

5. The composition of claim 4, wherein direct binding or indirect binding comprises basepairing interactions, non-canonical base pairing interactions, non-base pairing interactions, or a combination thereof.

6. The composition of claim 4 or 5, wherein direct binding comprises base pairing interactions between kissing loops or hypodiverse regions.

7. The composition of claim 4 or 5, wherein direct binding comprises non-canonical basepairing interactions, non-canonical base pairing interactions, non-base pairing interactions, or a combination thereof, between aptamer regions.

8. The composition of claim 4 or 5, wherein indirect binding comprises basepairing interactions through a nucleic acid bridge.

9. The composition of claim 4 or 5, wherein indirect binding comprises non-base pairing interactions between an aptamer and an aptamer target agent, or between two aptamers.

10. The composition of any one of embodiments 1 to 9, wherein the first, second, third, fourth, fifth and/or sixth dimerization domain does not comprise a cryptic splice acceptor.

11. The composition of any one of embodiments 1 to 10, comprising at least one pair of directly binding or indirectly binding aptamer sequence dimerization domains.

12. The composition of any one of embodiments 1 to 11, comprising at least one pair of kissing loop interaction dimerization domains.

13. The composition of any one of embodiments 1 to 12, wherein the target protein is a protein associated with disease, or a therapeutic protein.

14. The composition of embodiment 13, wherein the disease is a monogenic disease.

15. The composition of embodiment 14, wherein the therapeutic protein is a toxin.

16. The composition of any one of embodiments 13 to 15, wherein the disease and the target protein are one listed in Table 1.

17. The composition of any one of embodiments 1 to 16, wherein the first, second, third, and/or fourth RNA molecule further comprises a polyA tail at a 3′-end of the first, second, third, or fourth RNA molecule.

18. The composition of any one of embodiments 1 or 4 to 17, wherein the first RNA molecule further comprises one or both of a downstream intronic splice enhancer (DISE) 3′ to the splice donor and 5′ to the first dimerization domain, an intronic splice enhancer (ISE) 3′ to the splice donor and 5′ to the first dimerization domain; and/or
the second RNA molecule further comprises one or both of an ISE 3′ to the second dimerization domain and 5′ to the branch point sequence, and a DISE 3′ to the splice donor and 5′ to the dimerization domain;
or any combination thereof.

19. The composition of any one of embodiments 2 or 4 to 17, wherein the
the first RNA molecule further comprises a DISE 3′ to the first splice donor and 5′ to the first dimerization domain, an ISE 3′ to the first splice donor and 5′ to the first dimerization domain, or both;
the RNA molecule further comprises an ISE 3′ to the second dimerization domain and 5′ to the first branch point sequence, a DISE 3′ to the second splice donor and 5′ to the second dimerization domain, an ISE 3′ to the second splice donor and 5′ to the third dimerization domain, or any combination thereof; and/or
the third RNA molecule further comprises an ISE 3′ to the fourth dimerization domain and 5′ to the second branch point sequence;
or any combination thereof.

20. The composition of any one of embodiments 3 to 17, wherein
the first RNA molecule further comprises a DISE 3′ to the first splice donor and 5′ to the first dimerization domain, an ISE 3′ to the first splice donor and 5′ to the first dimerization domain, or both;
the second RNA molecule further comprises an ISE 3′ to the second dimerization domain and 5′ to the first branch point sequence, a DISE 3′ to the second splice donor and 5′ to the second dimerization domain, an ISE 3′ to the second splice donor and 5′ to the third dimerization domain, or any combination thereof;
the third RNA molecule further comprises an ISE 3′ to the fourth dimerization domain and 5′ to the second branch point sequence; and/or
the fourth RNA molecule further comprises an ISE 3′ to the fifth dimerization domain and 5′ to the third branch point sequence, a DISE 3′ to the third splice donor and 5′ to the fifth dimerization domain, an ISE 3′ to the third splice donor and 5′ to the sixth dimerization domain, or any combination thereof;
or any combination thereof.

24. The composition of any one of embodiments 1 to 23, wherein
the first and/or third RNA molecule further comprises a self-cleaving RNA sequence or an RNA-cleaving enzyme target sequence positioned anywhere 3′ to the splice donor such that it cleaves off the 3′ located polyadenylated tail to decrease or suppress protein fragment expression from a non-recombined RNA molecule;
the second and/or fourth RNA molecule further comprises a self-cleaving RNA sequence or an RNA-cleaving enzyme target sequence positioned anywhere 5′ to the branch point sequence such that it cleaves off the 5′ located RNA cap to decrease or suppress protein fragment expression from a non-recombined RNA molecule;
the second and/or fourth RNA molecule further comprises a start codon anywhere 5′ to the branch point sequence that is shifted relative to the open reading frame 3′ of the splice acceptor to decrease or suppress translation of a target protein fragment from a non-recombined RNA molecule;
the first and/or third RNA molecule further comprises a micro RNA target site anywhere 3′ to the splice donor such that an un-joined RNA fragment undergoes micro RNA dependent degradation once outside the nucleus;
the second and/or fourth RNA molecule further comprises a micro RNA target site anywhere 3′ to the coding sequence such that an un-joined RNA fragment undergoes micro RNA dependent degradation once outside the nucleus;
the first and/or third RNA molecule further comprises a sequence encoding a degron protein degradation tag anywhere 3′ to the splice donor such that it is in frame with the target protein open reading frame 5′ of the splice donor site such that an un-joined protein fragment is tagged for degradation;
the second and/or fourth RNA molecule further comprises a start codon and an in-frame degron protein degradation tag anywhere 5′ to the branch point sequence such that it is in frame with the target protein open reading frame 3′ of the splice acceptor site such that an un-joined protein fragment is tagged for degradation;
or any combination thereof.

25. A composition for expressing a target protein comprising: (a) a first synthetic DNA molecule that encodes the first RNA molecule of any one of embodiments 1 and 4 to 24, wherein the first synthetic DNA molecule comprises (i) a first promoter operably linked to a sequence encoding the first RNA molecule; and (b) a second synthetic DNA molecule that encodes the second RNA molecule of any one of embodiments 1 and 4 to 24, wherein the second synthetic DNA molecule comprises (i) a second promoter operably linked to a sequence encoding the second RNA molecule.

26. A composition for expressing a target protein comprising: (a) a first synthetic DNA molecule that encodes the first RNA molecule of any one of embodiments 2 and 4 to 24, wherein the first synthetic DNA molecule comprises (i) a first promoter operably linked to a sequence encoding the first RNA molecule; (b) a second synthetic DNA molecule that encodes the second RNA molecule of any one of embodiments 2 and 4 to 24, wherein the second synthetic DNA molecule comprises (i) a second promoter operably linked to a sequence encoding the second RNA molecule; and (c) a third synthetic DNA molecule that encodes the third RNA molecule of any one of embodiments 2 and 4 to 24, wherein the third synthetic DNA molecule comprises (i) a third promoter operably linked to a sequence encoding the third RNA molecule.

27. A composition for expressing a target protein comprising: (a) a first synthetic DNA molecule that encodes the first RNA molecule of any one of embodiments 3 and 4 to 24, wherein the first synthetic DNA molecule comprises (i) a first promoter operably linked to a sequence encoding the first RNA molecule; (b) a second synthetic DNA molecule that encodes the second RNA molecule of any one of embodiments 3 and 4 to 24, wherein the second synthetic DNA molecule comprises (i) a second promoter operably linked to a sequence encoding the second RNA molecule; (c) a third synthetic DNA molecule that encodes the third RNA molecule of any one of embodiments 3 and 4 to 24, wherein the third synthetic DNA molecule comprises (i) a third promoter operably linked to a sequence encoding the third RNA molecule; and (d) a fourth synthetic DNA molecule that encodes the fourth RNA molecule of any one of embodiments 3 and 4 to 24, wherein the fourth synthetic DNA molecule comprises (i) a fourth promoter operably linked to a sequence encoding the fourth RNA molecule.

28. The composition of any one of embodiments 25 to 27, wherein each promoter is independently selected.

29. The composition of any one of embodiments 25 to 28, wherein:
the first and second promoter are the same promoter;
the first and second promoter are different promoters;
the first, second, and third promoters are the same promoter;
the first, second, and third promoters are different promoters;
the first, second, third, and fourth promoters are the same promoter; or
the first, second, third and fourth promoters are different promoters.

30. The composition of any one of embodiments 25 to 29, wherein each of the first, second, third, and fourth promoter is independently selected from: a constitutive promoter; a tissue-specific promoter; and a promoter endogenous to the target protein.

31. A system for expressing a target protein comprising a composition of any one of embodiments to 30.

32. The system of embodiment 31, wherein when the system is introduced into a cell the RNA molecules are produced and recombine in the proper order, resulting in a full-length coding sequence of the target protein.

33. The system of embodiment 31 or 32, wherein each of the first and second RNA molecules (in a two-part system), each of the first, second and third RNA molecules (in a three-part system), or each of the first, second, third and fourth, RNA molecules (in a four-part system) is transcribed from a separate viral vector.

34. The system of any one of embodiments 31 to 33, wherein the viral vector is AAV.

35. The system of any one of embodiments 31 to 34, wherein the first, second, third, or fourth synthetic DNA molecule of the system each has a size independently selected from: about 2500 nt to about 5000 nt, 2,500 nt to about 2,750 nt, about 2,500 nt to about 3,000 nt, about 2,500 nt to about 3,250 nt, about 2,500 nt to about 3,500 nt, about 2,500 nt to about 3,750 nt, about 2,500 nt to about 4,000 nt, about 2,500 nt to about 4,250 nt, about 2,500 nt to about 4,500 nt, about 2,500 nt to about 4,750 nt, about 2,500 nt to about 5,000 nt, about 2,750 nt to about 3,000 nt, about 2,750 nt to about 3,250 nt, about 2,750 nt to about 3,500 nt, about 2,750 nt to about 3,750 nt, about 2,750 nt to about 4,000 nt, about 2,750 nt to about 4,250 nt, about 2,750 nt to about 4,500 nt, about 2,750 nt to about 4,750 nt, about 2,750 nt to about 5,000 nt, about 3,000 nt to about 3,250 nt, about 3,000 nt to about 3,500 nt, about 3,000 nt to about 3,750 nt, about 3,000 nt to about 4,000 nt, about 3,000 nt to about 4,250 nt, about 3,000 nt to about 4,500 nt, about 3,000 nt to about 4,750 nt, about 3,000 nt to about 5,000 nt, about 3,250 nt to about 3,500 nt, about 3,250 nt to about 3,750 nt, about 3,250 nt to about 4,000 nt, about 3,250 nt to about 4,250 nt, about 3,250 nt to about 4,500 nt, about 3,250 nt to about 4,750 nt, about 3,250 nt to about 5,000 nt, about 3,500 nt to about 3,750 nt, about 3,500 nt to about 4,000 nt, about 3,500 nt to about 4,250 nt, about 3,500 nt to about 4,500 nt, about 3,500 nt to about 4,750 nt, about 3,500 nt to about 5,000 nt, about 3,750 nt to about 4,000 nt, about 3,750 nt to about 4,250 nt, about 3,750 nt to about 4,500 nt, about 3,750 nt to about 4,750 nt, about 3,750 nt to about 5,000 nt, about 4,000 nt to about 4,250 nt, about 4,000 nt to about 4,500 nt, about 4,000 nt to about 4,750 nt, about 4,000 nt to about 5,000 nt, about 4,250 nt to about 4,500 nt, about 4,250 nt to about 4,750 nt, about 4,250 nt to about 5,000 nt, about 4,500 nt to about 4,750 nt, about 4,500 nt to about 5,000 nt, about 4,750 nt to about 5,000 nt, about 2,500 nt, about 2,750 nt, about 3,000 nt, about 3,250 nt, about 3,500 nt, about 3,750 nt, about 4,000 nt, about 4,250 nt, about 4,500 nt, about 4,750 nt, and about 5,000 nt.

36. The system of any one of embodiments 31 to 35, wherein the coding sequence for an N-terminal portion of the target protein (in a two, three, or four-part system), a middle portion of the target protein (in a three-part system), a first middle portion of the target protein (in a four-part system), or a C-terminal portion of the target protein (in a two, three, or four-part system) encoded by a synthetic DNA molecule of the system each has a size independently selected from: each has a size independently selected from: about 1,000 nt to about 4,500 nt, each has a size independently selected from: about 1,000 nt to about 1,500 nt, about 1,000 nt to about 2,000 nt, about 1,000 nt to about 2,500 nt, about 1,000 nt to about 3,000 nt, about 1,000 nt to about 3,500 nt, about 1,000 nt to about 4,000 nt, about 1,000 nt to about 4,500 nt, about 1,500 nt to about 2,000 nt, about 1,500 nt to about 2,500 nt, about 1,500 nt to about 3,000 nt, about 1,500 nt to about 3,500 nt, about 1,500 nt to about 4,000 nt, about 1,500 nt to about 4,500 nt, about 2,000 nt to about 2,500 nt, about 2,000 nt to about 3,000 nt, about 2,000 nt to about 3,500 nt, about 2,000 nt to about 4,000 nt, about 2,000 nt to about 4,500 nt, about 2,500 nt to about 3,000 nt, about 2,500 nt to about 3,500 nt, about 2,500 nt to about 4,000 nt, about 2,500 nt to about 4,500 nt, about 3,000 nt to about 3,500 nt, about 3,000 nt to about 4,000 nt, about 3,000 nt to about 4,500 nt, about 3,500 nt to about 4,000 nt, about 3,500 nt to about 4,500 nt, about 4,000 nt to about 4,500 nt, about 1,000 nt, about 1,500 nt, about 2,000 nt, about 2,500 nt, about 3,000 nt, about 3,500 nt, about 4,000 nt, or about 4,500 nt.

37. The system of any one of embodiments 31 to 36, wherein any one, two, three, or four RNA molecules encoded by any of the one, two, three, or four synthetic DNA molecules of the system, respectively, each has a size independently selected from: about 2500 to 4500 nt, about 2,500 nt to about 2,750 nt, about 2,500 nt to about 3,000 nt, about 2,500 nt to about 3,250 nt, about 2,500 nt to about 3,500 nt, about 2,500 nt to about 3,750 nt, about 2,500 nt to about 4,000 nt, about 2,500 nt to about 4,250 nt, about 2,500 nt to about 4,500 nt, about 2,750 nt to about 3,000 nt, about 2,750 nt to about 3,250 nt, about 2,750 nt to about 3,500 nt, about 2,750 nt to about 3,750 nt, about 2,750 nt to about 4,000 nt, about 2,750 nt to about 4,250 nt, about 2,750 nt to about 4,500 nt, about 3,000 nt to about 3,250 nt, about 3,000 nt to about 3,500 nt, about 3,000 nt to about 3,750 nt, about 3,000 nt to about 4,000 nt, about 3,000 nt to about 4,250 nt, about 3,000 nt to about 4,500 nt, about 3,250 nt to about 3,500 nt, about 3,250 nt to about 3,750 nt, about 3,250 nt to about 4,000 nt, about 3,250 nt to about 4,250 nt, about 3,250 nt to about 4,500 nt, about 3,500 nt to about 3,750 nt, about 3,500 nt to about 4,000 nt, about 3,500 nt to about 4,250 nt, about 3,500 nt to about 4,500 nt, about 3,750 nt to about 4,000 nt, about 3,750 nt to about 4,250 nt, about 3,750 nt to about 4,500 nt, about 4,000 nt to about 4,250 nt, about 4,000 nt to about 4,500 nt, about 4,250 nt to about 4,500 nt, about 2,500 nt, about 2,750 nt, about 3,000 nt, about 3,250 nt, about 3,500 nt, about 3,750 nt, about 4,000 nt, about 4,250 nt, and about 4,500 nt.

38. The system of any one of embodiments 31 to 37, the system comprising a composition of embodiment 25 and 28 to 30, wherein:
the first and second synthetic DNA molecules have a total size selected from about 5000 nt to about 10,000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,000 nt to about 8,500 nt, about 5,000 nt to about 9,000 nt, about 5,000 nt to about 9,500 nt, about 5,000 nt to about 10,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 5,500 nt to about 8,500 nt, about 5,500 nt to about 9,000 nt, about 5,500 nt to about 9,500 nt, about 5,500 nt to about 10,000 nt, about 6,000 nt to about 6,500 nt, about 6,000 nt to about 7,000 nt, about 6,000 nt to about 7,500 nt, about 6,000 nt to about 8,000 nt, about 6,000 nt to about 8,500 nt, about 6,000 nt to about 9,000 nt, about 6,000 nt to about 9,500 nt, about 6,000 nt to about 10,000 nt, about 6,500 nt to about 7,000 nt, about 6,500 nt to about 7,500 nt, about 6,500 nt to about 8,000 nt, about 6,500 nt to about 8,500 nt, about 6,500 nt to about 9,000 nt, about 6,500 nt to about 9,500 nt, about 6,500 nt to about 10,000 nt, about 7,000 nt to about 7,500 nt, about 7,000 nt to about 8,000 nt, about 7,000 nt to about 8,500 nt, about 7,000 nt to about 9,000 nt, about 7,000 nt to about 9,500 nt, about 7,000 nt to about 10,000 nt, about 7,500 nt to about 8,000 nt, about 7,500 nt to about 8,500 nt, about 7,500 nt to about 9,000 nt, about 7,500 nt to about 9,500 nt, about 7,500 nt to about 10,000 nt, about 8,000 nt to about 8,500 nt, about 8,000 nt to about 9,000 nt, about 8,000 nt to about 9,500 nt, about 8,000 nt to about 10,000 nt, about 8,500 nt to about 9,000 nt, about 8,500 nt to about 9,500 nt, about 8,500 nt to about 10,000 nt, about 9,000 nt to about 9,500 nt, about 9,000 nt to about 10,000 nt, about 9,500 nt to about 10,000 nt, about 5,000 nt, about 5,500 nt, about 6,000 nt, about 6,500 nt, about 7,000 nt, about 7,500 nt, about 8,000 nt, about 8,500 nt, about 9,000 nt, about 9,500 nt, and about 10,000 nt;
   the total target protein coding sequence size is selected from about 2000 nt to about 8000 nt, about 2,000 nt to about 3,000 nt, about 2,000 nt to about 3,500 nt, about 2,000 nt to about 4,000 nt, about 2,000 nt to about 4,500 nt, about 2,000 nt to about 5,000 nt, about 2,000 nt to about 5,500 nt, about 2,000 nt to about 6,000 nt, about 2,000 nt to about 6,500 nt, about 2,000 nt to about 7,000 nt, about 2,000 nt to about 7,500 nt, about 2,000 nt to about 8,000 nt, about 3,000 nt to about 3,500 nt, about 3,000 nt to about 4,000 nt, about 3,000 nt to about 4,500 nt, about 3,000 nt to about 5,000 nt, about 3,000 nt to about 5,500 nt, about 3,000 nt to about 6,000 nt, about 3,000 nt to about 6,500 nt, about 3,000 nt to about 7,000 nt, about 3,000 nt to about 7,500 nt, about 3,000 nt to about 8,000 nt, about 3,500 nt to about 4,000 nt, about 3,500 nt to about 4,500 nt, about 3,500 nt to about 5,000 nt, about 3,500 nt to about 5,500 nt, about 3,500 nt to about 6,000 nt, about 3,500 nt to about 6,500 nt, about 3,500 nt to about 7,000 nt, about 3,500 nt to about 7,500 nt, about 3,500 nt to about 8,000 nt, about 4,000 nt to about 4,500 nt, about 4,000 nt to about 5,000 nt, about 4,000 nt to about 5,500 nt, about 4,000 nt to about 6,000 nt, about 4,000 nt to about 6,500 nt, about 4,000 nt to about 7,000 nt, about 4,000 nt to about 7,500 nt, about 4,000 nt to about 8,000 nt, about 4,500 nt to about 5,000 nt, about 4,500 nt to about 5,500 nt, about 4,500 nt to about 6,000 nt, about 4,500 nt to about 6,500 nt, about 4,500 nt to about 7,000 nt, about 4,500 nt to about 7,500 nt, about 4,500 nt to about 8,000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 6,000 nt to about 6,500 nt, about 6,000 nt to about 7,000 nt, about 6,000 nt to about 7,500 nt, about 6,000 nt to about 8,000 nt, about 6,500 nt to about 7,000 nt, about 6,500 nt to about 7,500 nt, about 6,500 nt to about 8,000 nt, about 7,000 nt to about 7,500 nt, about 7,000 nt to about 8,000 nt, or about 7,500 nt to about 8,000 nt, the total target protein coding sequence is about 2,000 nt, about 3,000 nt, about 3,500 nt, about 4,000 nt, about 4,500 nt, about 5,000 nt, about 5,500 nt, about 6,000 nt, about 6,500 nt, about 7,000 nt, about 7,500 nt, and about 8,000 nt; and/or
   the summed size of the RNA molecules encoded by the two synthetic DNA molecules is about 5,000 nt to about 9000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,000 nt to about 8,500 nt, about 5,000 nt to about 9,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 5,500 nt to about 8,500 nt, about 5,500 nt to about 9,000 nt, about 6,000 nt to about 6,500 nt, about 6,000 nt to about 7,000 nt, about 6,000 nt to about 7,500 nt, about 6,000 nt to about 8,000 nt, about 6,000 nt to about 8,500 nt, about 6,000 nt to about 9,000 nt, about 6,500 nt to about 7,000 nt, about 6,500 nt to about 7,500 nt, about 6,500 nt to about 8,000 nt, about 6,500 nt to about 8,500 nt, about 6,500 nt to about 9,000 nt, about 7,000 nt to about 7,500 nt, about 7,000 nt to about 8,000 nt, about 7,000 nt to about 8,500 nt, about 7,000 nt to about 9,000 nt, about 7,500 nt to about 8,000 nt, about 7,500 nt to about 8,500 nt, about 7,500 nt to about 9,000 nt, about 8,000 nt to about 8,500 nt, about 8,000 nt to about 9,000 nt, about 8,500 nt to about 9,000 nt, about 5,000 nt, about 5,500 nt, about 6,000 nt, about 6,500 nt, about 7,000 nt, about 7,500 nt, about 8,000 nt, about 8,500 nt, or about 9,000 nt.

39. The system of any one of embodiments 31 to 36, the system comprising a composition of any one of embodiments 26 and 28 to 30, wherein:
the first, second, and third synthetic DNA molecules have a total size of about 7500 nt to about 15,000 nt, about 7,500 nt to about 8,500 nt, about 7,500 nt to about 9,500 nt, about 7,500 nt to about 10,000 nt, about 7,500 nt to about 10,500 nt, about 7,500 nt to about 11,000 nt, about 7,500 nt to about 11,500 nt, about 7,500 nt to about 12,000 nt, about 7,500 nt to about 12,500 nt, about 7,500 nt to about 13,000 nt, about 7,500 nt to about 14,000 nt, about 7,500 nt to about 15,000 nt, about 8,500 nt to about 9,500 nt, about 8,500 nt to about 10,000 nt, about 8,500 nt to about 10,500 nt, about 8,500 nt to about 11,000 nt, about 8,500 nt to about 11,500 nt, about 8,500 nt to about 12,000 nt, about 8,500 nt to about 12,500 nt, about 8,500 nt to about 13,000 nt, about 8,500 nt to about 14,000 nt, about 8,500 nt to about 15,000 nt, about 9,500 nt to about 10,000 nt, about 9,500 nt to about 10,500 nt, about 9,500 nt to about 11,000 nt, about 9,500 nt to about 11,500 nt, about 9,500 nt to about 12,000 nt, about 9,500 nt to about 12,500 nt, about 9,500 nt to about 13,000 nt, about 9,500 nt to about 14,000 nt, about 9,500 nt to about 15,000 nt, about 10,000 nt to about 10,500 nt, about 10,000 nt to about 11,000 nt, about 10,000 nt to about 11,500 nt, about 10,000 nt to about 12,000 nt, about 10,000 nt to about 12,500 nt, about 10,000 nt to about 13,000 nt, about 10,000 nt to about 14,000 nt, about 10,000 nt to about 15,000 nt, about 10,500 nt to about 11,000 nt, about 10,500 nt to about 11,500 nt, about 10,500 nt to about 12,000 nt, about 10,500 nt to about 12,500 nt, about 10,500 nt to about 13,000 nt, about 10,500 nt to about 14,000 nt, about 10,500 nt to about 15,000 nt, about 11,000 nt to about 11,500 nt, about 11,000 nt to about 12,000 nt, about 11,000 nt to about 12,500 nt, about 11,000 nt to about 13,000 nt, about 11,000 nt to about 14,000 nt, about 11,000 nt to about 15,000 nt, about 11,500 nt to about 12,000 nt, about 11,500 nt to about 12,500 nt, about 11,500 nt to about 13,000 nt, about 11,500 nt to about 14,000 nt, about 11,500 nt to about 15,000 nt, about 12,000 nt to about 12,500 nt, about 12,000 nt to about 13,000 nt, about 12,000 nt to about 14,000 nt, about 12,000 nt to about 15,000 nt, about 12,500 nt to about 13,000 nt, about 12,500 nt to about 14,000 nt, about 12,500 nt to about 15,000 nt, about 13,000 nt to about 14,000 nt, about 13,000 nt to about 15,000 nt, or about 14,000 nt to about 15,000 nt, about 7,500 nt, about 8,500 nt, about 9,500 nt, about 10,000 nt, about 10,500 nt, about 11,000 nt, about 11,500 nt, about 12,000 nt, about 12,500 nt, about 13,000 nt, about 14,000 nt, or about 15,000 nt;
the total target protein coding sequence about 3000 nt to about 12,000 nt, about 3,000 nt to about 4,000 nt, about 3,000 nt to about 5,000 nt, about 3,000 nt to about 6,000 nt, about 3,000 nt to about 7,000 nt, about 3,000 nt to about 7,500 nt, about 3,000 nt to about 8,000 nt, about 3,000 nt to about 8,500 nt, about 3,000 nt to about 9,000 nt, about 3,000 nt to about 1,000 nt, about 3,000 nt to about 11,000 nt, about 3,000 nt to about 12,000 nt, about 4,000 nt to about 5,000 nt, about 4,000 nt to about 6,000 nt, about 4,000 nt to about 7,000 nt, about 4,000 nt to about 7,500 nt, about 4,000 nt to about 8,000 nt, about 4,000 nt to about 8,500 nt, about 4,000 nt to about 9,000 nt, about 4,000 nt to about 1,000 nt, about 4,000 nt to about 11,000 nt, about 4,000 nt to about 12,000 nt, about 5,000 nt to about 6,000 nt, about 5,000 nt to about 7,000 nt, about 5,000 nt to about 7,500 nt, about 5,000 nt to about 8,000 nt, about 5,000 nt to about 8,500 nt, about 5,000 nt to about 9,000 nt, about 5,000 nt to about 1,000 nt, about 5,000 nt to about 11,000 nt, about 5,000 nt to about 12,000 nt, about 6,000 nt to about 7,000 nt, about 6,000 nt to about 7,500 nt, about 6,000 nt to about 8,000 nt, about 6,000 nt to about 8,500 nt, about 6,000 nt to about 9,000 nt, about 6,000 nt to about 1,000 nt, about 6,000 nt to about 11,000 nt, about 6,000 nt to about 12,000 nt, about 7,000 nt to about 7,500 nt, about 7,000 nt to about 8,000 nt, about 7,000 nt to about 8,500 nt, about 7,000 nt to about 9,000 nt, about 7,000 nt to about 1,000 nt, about 7,000 nt to about 11,000 nt, about 7,000 nt to about 12,000 nt, about 7,500 nt to about 8,000 nt, about 7,500 nt to about 8,500 nt, about 7,500 nt to about 9,000 nt, about 7,500 nt to about 1,000 nt, about 7,500 nt to about 11,000 nt, about 7,500 nt to about 12,000 nt, about 8,000 nt to about 8,500 nt, about 8,000 nt to about 9,000 nt, about 8,000 nt to about 1,000 nt, about 8,000 nt to about 11,000 nt, about 8,000 nt to about 12,000 nt, about 8,500 nt to about 9,000 nt, about 8,500 nt to about 1,000 nt, about 8,500 nt to about 11,000 nt, about 8,500 nt to about 12,000 nt, about 9,000 nt to about 1,000 nt, about 9,000 nt to about 11,000 nt, about 9,000 nt to about 12,000 nt, about 1,000 nt to about 11,000 nt, about 1,000 nt to about 12,000 nt, about 11,000 nt to about 12,000 nt, about 3,000 nt, about 4,000 nt, about 5,000 nt, about 6,000 nt, about 7,000 nt, about 7,500 nt, about 8,000 nt, about 8,500 nt, about 9,000 nt, about 1,000 nt, about 11,000 nt, or about 12,000 nt; and/or
the summed size of the RNA molecules encoded by the three synthetic DNA molecules is about 7500 nt to about 13,500 nt, about 7,500 nt to about 8,500 nt, about 7,500 nt to about 9,000 nt, about 7,500 nt to about 9,500 nt, about 7,500 nt to about 10,000 nt, about 7,500 nt to about 10,500 nt, about 7,500 nt to about 11,000 nt, about 7,500 nt to about 11,500 nt, about 7,500 nt to about 12,000 nt, about 7,500 nt to about 12,500 nt, about 7,500 nt to about 13,000 nt, about 7,500 nt to about 13,500 nt, about 8,500 nt to about 9,000 nt, about 8,500 nt to about 9,500 nt, about 8,500 nt to about 10,000 nt, about 8,500 nt to about 10,500 nt, about 8,500 nt to about 11,000 nt, about 8,500 nt to about 11,500 nt, about 8,500 nt to about 12,000 nt, about 8,500 nt to about 12,500 nt, about 8,500 nt to about 13,000 nt, about 8,500 nt to about 13,500 nt, about 9,000 nt to about 9,500 nt, about 9,000 nt to about 10,000 nt, about 9,000 nt to about 10,500 nt, about 9,000 nt to about 11,000 nt, about 9,000 nt to about 11,500 nt, about 9,000 nt to about 12,000 nt, about 9,000 nt to about 12,500 nt, about 9,000 nt to about 13,000 nt, about 9,000 nt to about 13,500 nt, about 9,500 nt to about 10,000 nt, about 9,500 nt to about 10,500 nt, about 9,500 nt to about 11,000 nt, about 9,500 nt to about 11,500 nt, about 9,500 nt to about 12,000 nt, about 9,500 nt to about 12,500 nt, about 9,500 nt to about 13,000 nt, about 9,500 nt to about 13,500 nt, about 10,000 nt to about 10,500 nt, about 10,000 nt to about 11,000 nt, about 10,000 nt to about 11,500 nt, about 10,000 nt to about 12,000 nt, about 10,000 nt to about 12,500 nt, about 10,000 nt to about 13,000 nt, about 10,000 nt to about 13,500 nt, about 10,500 nt to about 11,000 nt, about 10,500 nt to about 11,500 nt, about 10,500 nt to about 12,000 nt, about 10,500 nt to about 12,500 nt, about 10,500 nt to about 13,000 nt, about 10,500 nt to about 13,500 nt, about 11,000 nt to about 11,500 nt, about 11,000 nt to about 12,000 nt, about 11,000 nt to about 12,500 nt, about 11,000 nt to about 13,000 nt, about 11,000 nt to about 13,500 nt, about 11,500 nt to about 12,000 nt, about 11,500 nt to about 12,500 nt, about 11,500 nt to about 13,000 nt, about 11,500 nt to about 13,500 nt, about 12,000 nt to about 12,500 nt, about 12,000 nt to about 13,000 nt, about 12,000 nt to about 13,500 nt, about 12,500 nt to about 13,000 nt, about 12,500 nt to about 13,500 nt, about 13,000 nt to about 13,500 nt about 7,500 nt, about 8,500 nt, about 9,000 nt, about 9,500 nt, about 10,000 nt, about 10,500 nt, about 11,000 nt, about 11,500 nt, about 12,000 nt, about 12,500 nt, about 13,000 nt, or about 13,500 nt.

40. The system of any one of embodiments 31 to 36, the system comprising a composition of any one of embodiments 27 and 28 to 30, wherein:
the first, second, third and fourth synthetic DNA molecules have a total size of about 10,000 nt to about 20,000 nt, about 10,000 nt to about 11,000 nt, about 10,000 nt to about 12,000 nt, about 10,000 nt to about 13,000 nt, about 10,000 nt to about 14,000 nt, about 10,000 nt to about 15,000 nt, about 10,000 nt to about 16,000 nt, about 10,000 nt to about 17,000 nt, about 10,000 nt to about 18,000 nt, about 10,000 nt to about 19,000 nt, about 10,000 nt to about 20,000 nt, about 11,000 nt to about 12,000 nt, about 11,000 nt to about 13,000 nt, about 11,000 nt to about 14,000 nt, about 11,000 nt to about 15,000 nt, about 11,000 nt to about 16,000 nt, about 11,000 nt to about 17,000 nt, about 11,000 nt to about 18,000 nt, about 11,000 nt to about 19,000 nt, about 11,000 nt to about 20,000 nt, about 12,000 nt to about 13,000 nt, about 12,000 nt to about 14,000 nt, about 12,000 nt to about 15,000 nt, about 12,000 nt to about 16,000 nt, about 12,000 nt to about 17,000 nt, about 12,000 nt to about 18,000 nt, about 12,000 nt to about 19,000 nt, about 12,000 nt to about 20,000 nt, about 13,000 nt to about 14,000 nt, about 13,000 nt to about 15,000 nt, about 13,000 nt to about 16,000 nt, about 13,000 nt to about 17,000 nt, about 13,000 nt to about 18,000 nt, about 13,000 nt to about 19,000 nt, about 13,000 nt to about 20,000 nt, about 14,000 nt to about 15,000 nt, about 14,000 nt to about 16,000 nt, about 14,000 nt to about 17,000 nt, about 14,000 nt to about 18,000 nt, about 14,000 nt to about 19,000 nt, about 14,000 nt to about 20,000 nt, about 15,000 nt to about 16,000 nt, about 15,000 nt to about 17,000 nt, about 15,000 nt to about 18,000 nt, about 15,000 nt to about 19,000 nt, about 15,000 nt to about 20,000 nt, about 16,000 nt to about 17,000 nt, about 16,000 nt to about 18,000 nt, about 16,000 nt to about 19,000 nt, about 16,000 nt to about 20,000 nt, about 17,000 nt to about 18,000 nt, about 17,000 nt to about 19,000 nt, about 17,000 nt to about 20,000 nt, about 18,000 nt to about 19,000 nt, about 18,000 nt to about 20,000 nt, about 19,000 nt to about 20,000 nt about 10,000 nt, about 11,000 nt, about 12,000 nt, about 13,000 nt, about 14,000 nt, about 15,000 nt, about 16,000 nt, about 17,000 nt, about 18,000 nt, about 19,000 nt, or about 20,000 nt;
the total target protein coding sequence is about 4000 nt to about 16,000 nt, about 5,000 nt to about 6,000 nt, about 5,000 nt to about 7,000 nt, about 5,000 nt to about 8,000 nt, about 5,000 nt to about 9,000 nt, about 5,000 nt to about 10,000 nt, about 5,000 nt to about 11,000 nt, about 5,000 nt to about 12,000 nt, about 5,000 nt to about 13,000 nt, about 5,000 nt to about 14,000 nt, about 5,000 nt to about 15,000 nt, about 5,000 nt to about 16,000 nt, about 6,000 nt to about 7,000 nt, about 6,000 nt to about 8,000 nt, about 6,000 nt to about 9,000 nt, about 6,000 nt to about 10,000 nt, about 6,000 nt to about 11,000 nt, about 6,000 nt to about 12,000 nt, about 6,000 nt to about 13,000 nt, about 6,000 nt to about 14,000 nt, about 6,000 nt to about 15,000 nt, about 6,000 nt to about 16,000 nt, about 7,000 nt to about 8,000 nt, about 7,000 nt to about 9,000 nt, about 7,000 nt to about 10,000 nt, about 7,000 nt to about 11,000 nt, about 7,000 nt to about 12,000 nt, about 7,000 nt to about 13,000 nt, about 7,000 nt to about 14,000 nt, about 7,000 nt to about 15,000 nt, about 7,000 nt to about 16,000 nt, about 8,000 nt to about 9,000 nt, about 8,000 nt to about 10,000 nt, about 8,000 nt to about 11,000 nt, about 8,000 nt to about 12,000 nt, about 8,000 nt to about 13,000 nt, about 8,000 nt to about 14,000 nt, about 8,000 nt to about 15,000 nt, about 8,000 nt to about 16,000 nt, about 9,000 nt to about 10,000 nt, about 9,000 nt to about 11,000 nt, about 9,000 nt to about 12,000 nt, about 9,000 nt to about 13,000 nt, about 9,000 nt to about 14,000 nt, about 9,000 nt to about 15,000 nt, about 9,000 nt to about 16,000 nt, about 10,000 nt to about 11,000 nt, about 10,000 nt to about 12,000 nt, about 10,000 nt to about 13,000 nt, about 10,000 nt to about 14,000 nt, about 10,000 nt to about 15,000 nt, about 10,000 nt to about 16,000 nt, about 11,000 nt to about 12,000 nt, about 11,000 nt to about 13,000 nt, about 11,000 nt to about 14,000 nt, about 11,000 nt to about 15,000 nt, about 11,000 nt to about 16,000 nt, about 12,000 nt to about 13,000 nt, about 12,000 nt to about 14,000 nt, about 12,000 nt to about 15,000 nt, about 12,000 nt to about 16,000 nt, about 13,000 nt to about 14,000 nt, about 13,000 nt to about 15,000 nt, about 13,000 nt to about 16,000 nt, about 14,000 nt to about 15,000 nt, about 14,000 nt to about 16,000 nt, or about 15,000 nt to about 16,000 nt, the total target protein coding sequence is about 5,000 nt, about 6,000 nt, about 7,000 nt, about 8,000 nt, about 9,000 nt, about 10,000 nt, about 11,000 nt, about 12,000 nt, about 13,000 nt, about 14,000 nt, about 15,000 nt, about 16,000 nt about 5,000 nt, about 6,000 nt, about 7,000 nt, about 8,000 nt, about 9,000 nt, about 10,000 nt, about 11,000 nt, about 12,000 nt, about 13,000 nt, about 14,000 nt, or about 15,000 nt; and/or
the summed size of the RNA molecules encoded by the four synthetic DNA molecules is about 10,000 nt to about 18,000 nt, about 10,000 nt to about 11,000 nt, about 10,000 nt to about 12,000 nt, about 10,000 nt to about 13,000 nt, about 10,000 nt to about 14,000 nt, about 10,000 nt to about 15,000 nt, about 10,000 nt to about 16,000 nt, about 10,000 nt to about 17,000 nt, about 10,000 nt to about 18,000 nt, about 11,000 nt to about 12,000 nt, about 11,000 nt to about 13,000 nt, about 11,000 nt to about 14,000 nt, about 11,000 nt to about 15,000 nt, about 11,000 nt to about 16,000 nt, about 11,000 nt to about 17,000 nt, about 11,000 nt to about 18,000 nt, about 12,000 nt to about 13,000 nt, about 12,000 nt to about 14,000 nt, about 12,000 nt to about 15,000 nt, about 12,000 nt to about 16,000 nt, about 12,000 nt to about 17,000 nt, about 12,000 nt to about 18,000 nt, about 13,000 nt to about 14,000 nt, about 13,000 nt to about 15,000 nt, about 13,000 nt to about 16,000 nt, about 13,000 nt to about 17,000 nt, about 13,000 nt to about 18,000 nt, about 14,000 nt to about 15,000 nt, about 14,000 nt to about 16,000 nt, about 14,000 nt to about 17,000 nt, about 14,000 nt to about 18,000 nt, about 15,000 nt to about 16,000 nt, about 15,000 nt to about 17,000 nt, about 15,000 nt to about 18,000 nt, about 16,000 nt to about 17,000 nt, about 16,000 nt to about 18,000 nt, about 17,000 nt to about 18,000 nt, about 10,000 nt, about 11,000 nt, about 12,000 nt, about 13,000 nt, about 14,000 nt, about 15,000 nt, about 16,000 nt, about 17,000 nt, or about 18,000 nt.

41. The system of any one of embodiments 31 to 40, wherein the first dimerization domain and the second dimerization domain, the third dimerization domain and the fourth dimerization domain, and/or the fifth dimerization domain and the sixth dimerization domain, are each no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150, 200, 250, 300, 400, or 500 nt; and the system has a recombination efficiency of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

42. The system of any one of embodiments 31 to 41, wherein each dimerization domain is no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150, 200, 250, 300, 400, or 500 nt; and the system has a recombination efficiency of at least 20%, at least 30%, at least 40%, at least 50%, at least 60%, at least 70%, at least 75%, at least 80%, at least 90%, or about 100%.

43. The system of any one of embodiments 31 to 42, wherein the RNA recombination efficiency is about 10% to about 100%, about 10% to about 20%, about 10% to about 30%, about 10% to about 35%, about 10% to about 40%, about 10% to about 45%, about 10% to about 50%, about 10% to about 55%, about 10% to about 60%, about 10% to about 70%, about 10% to about 80%, about 10% to about 90%, about 20% to about 30%, about 20% to about 35%, about 20% to about 40%, about 20% to about 45%, about 20% to about 50%, about 20% to about 55%, about 20% to about 60%, about 20% to about 70%, about 20% to about 80%, about 20% to about 90%, about 30% to about 35%, about 30% to about 40%, about 30% to about 45%, about 30% to about 50%, about 30% to about 55%, about 30% to about 60%, about 30% to about 70%, about 30% to about 80%, about 30% to about 90%, about 35% to about 40%, about 35% to about 45%, about 35% to about 50%, about 35% to about 55%, about 35% to about 60%, about 35% to about 70%, about 35% to about 80%, about 35% to about 90%, about 40% to about 45%, about 40% to about 50%, about 40% to about 55%, about 40% to about 60%, about 40% to about 70%, about 40% to about 80%, about 40% to about 90%, about 45% to about 50%, about 45% to about 55%, about 45% to about 60%, about 45% to about 70%, about 45% to about 80%, about 45% to about 90%, about 50% to about 55%, about 50% to about 60%, about 50% to about 70%, about 50% to about 80%, about 50% to about 90%, about 55% to about 60%, about 55% to about 70%, about 55% to about 80%, about 55% to about 90%, about 60% to about 70%, about 60% to about 80%, about 60% to about 90%, about 70% to about 80%, about 70% to about 90%, about 80% to about 90%, about 10%, about 20%, about 30%, about 35%, about 40%, about 45%, about 50%, about 55%, about 60%, about 70%, about 80%, about 90%, about 95%, or about 100%.

44. A composition comprising a system of any one of embodiments 31 to 43.

45. The composition of embodiment 44, wherein the composition comprises first, second, third and optionally fourth RNA molecules, each encoding at least a portion of dystrophin, factor 8, ABCA4, or MYO7A.

46. A kit comprising the system of any one of embodiments 31 to 43, or composition of any one of embodiments 44 and 45, wherein any of the synthetic first, second, third and fourth nucleic acid molecules can be in separate containers, and optionally further comprising a buffer such as a pharmaceutically acceptable carrier.

47. A method of expressing a target protein in a cell, comprising:
introducing the system of any one of embodiments 31 to 43, or composition of any one of embodiments 44 and 45, into a cell, and expressing the first and second, first, second, and third, or first, second, third and fourth RNA molecules in the cell, wherein the target protein is produced in the cell.

48. The method of embodiment 47, wherein the cell is in a subject, and introducing comprises administering a therapeutically effective amount the system to the subject.

49. The method of embodiment 48, wherein the method treats a genetic disease caused by a mutation in a gene encoding the target protein in the subject, wherein the method results in expression of functional target protein in the subject.

50. The method of embodiment 49, wherein
the genetic disease is Duchenne muscular dystrophy and the target protein is dystrophin;
the genetic disease is Hemophilia A and the target protein is F8;
the genetic disease is Stargardt disease and the target protein is ABCA4; or
the genetic disease is Usher syndrome and the target protein is MYO7A.

51. A system of any one of embodiments 31 to 43, a composition of any one of embodiments 1 to 24, 44 and 45, a kit of embodiment 46, or a method of any one of embodiments 47 to 50, wherein one, two, three, or four RNA molecules comprise at least 80%, at least 85%, at least 90%, at least 95%, at least 98%, at least 99%, or 100% sequence identity to a synthetic intron provided in any one of SEQ ID NOS: 1, 2, 3, 4, 5, 6, 7, 8, 9, 10, 11, 12, 13, 14, 15, 16, 17, 18, 19, 20, 21, 22, 23, 24, 25, 145, 146, 147, 148, 155, 156, 157, 158, 159, 160, 161, 162, 163, 164, 165, and 166.

52. A system of any one of embodiments 31 to 43, and 51, a composition of any one of embodiments 1-24, 44 and 45, a kit of embodiment 46, or a method of any one of embodiments 47 to 50, wherein one, two, three, or four RNA molecules comprise a synthetic intron selected from: nt 3703 to 3975 of SEQ ID NO: 20, nt 1 to 228 of SEQ ID NO: 21, nt 3703 to 3975 of SEQ ID NO: 22, nt 1 to 225 of SEQ ID NO: 23, nt 3560 to 3828 of SEQ ID NO: 24, and nt 1-225 of SEQ ID NO: 25.

53. A system of any of embodiments 31 to 43, 51, and 52, a composition of any one of embodiments 1 to 24, 44 and 45, or a method of any one of embodiments 47 to 50, wherein the one, two, three, or four RNA molecules further comprise a portion of a protein coding sequence.

54. A system of any of embodiments 31 to 43, and 51 to 53, a composition of any one of embodiments 1 to 24, 44 and 45, or a method of any one of embodiments 47 to 50, wherein the portion of the protein coding sequence comprises an N-terminal half, an N-terminal third, a middle portion, a first middle portion, a C-terminal half, or a C-terminal third of the protein coding sequence.

55. A system of any one of embodiments 31 to 43 and 51 to 54, a composition of any one of embodiments 1 to 24, 44 and 45, or a method of any one of embodiments 47 to 50, comprising: (a) a first RNA molecule, the RNA molecule comprising from 5′ to 3′: (i) a coding sequence for an N-terminal portion of the target protein; (ii) a splice donor; (ii-2) a DISE, an ISE, or both; and (iii) a first dimerization domain; and (b) a second RNA molecule, the RNA molecule comprising from 5′ to 3′: (i) a second dimerization domain, wherein the second dimerization domain binds to the first dimerization domain; (i-2) at least one ISE sequences; (ii) a branch point sequence; (iii) a polypyrimidine tract; (iv) a splice acceptor; and (v) a coding sequence for a C-terminal portion of the target protein.

56. A system of any one of embodiments 31 to 43 and 51 to 55, a composition of any one of embodiments 1 to 24, 44 and 45, or a method of any one of embodiments 47 to 50, comprising: (a) a first RNA molecule, the RNA molecule comprising from 5′ to 3′: (i) a coding sequence for an N-terminal portion of the target protein; (ii) a splice donor; (ii-2) a DISE, an ISE, and an ISE; and (iii) a first dimerization domain; and (b) a second RNA molecule, the RNA molecule comprising from 5′ to 3′: (i) a second dimerization domain, wherein the second dimerization domain binds to the first dimerization domain; (i-2) three ISE sequences; (ii) a branch point sequence; (iii) a polypyrimidine tract; (iv) a splice acceptor; and (v) a coding sequence for a C-terminal portion of the target protein.

57. The composition of any one of embodiments 1 and 4-24, wherein any one or two of the two RNA molecules, or the composition of any one of embodiments 2 and 4-24, wherein any one, two or three of the three RNA molecules, or the composition of any one of embodiments 3 and 4-24, wherein any one, two, three, or four of the four RNA molecules, each has a size independently selected from: about 2500 to 4500 nt, about 2,500 nt to about 2,750 nt, about 2,500 nt to about 3,000 nt, about 2,500 nt to about 3,250 nt, about 2,500 nt to about 3,500 nt, about 2,500 nt to about 3,750 nt, about 2,500 nt to about 4,000 nt, about 2,500 nt to about 4,250 nt, about 2,500 nt to about 4,500 nt, about 2,750 nt to about 3,000 nt, about 2,750 nt to about 3,250 nt, about 2,750 nt to about 3,500 nt, about 2,750 nt to about 3,750 nt, about 2,750 nt to about 4,000 nt, about 2,750 nt to about 4,250 nt, about 2,750 nt to about 4,500 nt, about 3,000 nt to about 3,250 nt, about 3,000 nt to about 3,500 nt, about 3,000 nt to about 3,750 nt, about 3,000 nt to about 4,000 nt, about 3,000 nt to about 4,250 nt, about 3,000 nt to about 4,500 nt, about 3,250 nt to about 3,500 nt, about 3,250 nt to about 3,750 nt, about 3,250 nt to about 4,000 nt, about 3,250 nt to about 4,250 nt, about 3,250 nt to about 4,500 nt, about 3,500 nt to about 3,750 nt, about 3,500 nt to about 4,000 nt, about 3,500 nt to about 4,250 nt, about 3,500 nt to about 4,500 nt, about 3,750 nt to about 4,000 nt, about 3,750 nt to about 4,250 nt, about 3,750 nt to about 4,500 nt, about 4,000 nt to about 4,250 nt, about 4,000 nt to about 4,500 nt, about 4,250 nt to about 4,500 nt, about 2,500 nt, about 2,750 nt, about 3,000 nt, about 3,250 nt, about 3,500 nt, about 3,750 nt, about 4,000 nt, about 4,250 nt, and about 4,500 nt.

58. The composition of any one of embodiments 1 and 4-24, wherein:
the total target protein coding sequence is about 2000 nt to about 8000 nt, about 2,000 nt to about 3,000 nt, about 2,000 nt to about 3,500 nt, about 2,000 nt to about 4,000 nt, about 2,000 nt to about 4,500 nt, about 2,000 nt to about 5,000 nt, about 2,000 nt to about 5,500 nt, about 2,000 nt to about 6,000 nt, about 2,000 nt to about 6,500 nt, about 2,000 nt to about 7,000 nt, about 2,000 nt to about 7,500 nt, about 2,000 nt to about 8,000 nt, about 3,000 nt to about 3,500 nt, about 3,000 nt to about 4,000 nt, about 3,000 nt to about 4,500 nt, about 3,000 nt to about 5,000 nt, about 3,000 nt to about 5,500 nt, about 3,000 nt to about 6,000 nt, about 3,000 nt to about 6,500 nt, about 3,000 nt to about 7,000 nt, about 3,000 nt to about 7,500 nt, about 3,000 nt to about 8,000 nt, about 3,500 nt to about 4,000 nt, about 3,500 nt to about 4,500 nt, about 3,500 nt to about 5,000 nt, about 3,500 nt to about 5,500 nt, about 3,500 nt to about 6,000 nt, about 3,500 nt to about 6,500 nt, about 3,500 nt to about 7,000 nt, about 3,500 nt to about 7,500 nt, about 3,500 nt to about 8,000 nt, about 4,000 nt to about 4,500 nt, about 4,000 nt to about 5,000 nt, about 4,000 nt to about 5,500 nt, about 4,000 nt to about 6,000 nt, about 4,000 nt to about 6,500 nt, about 4,000 nt to about 7,000 nt, about 4,000 nt to about 7,500 nt, about 4,000 nt to about 8,000 nt, about 4,500 nt to about 5,000 nt, about 4,500 nt to about 5,500 nt, about 4,500 nt to about 6,000 nt, about 4,500 nt to about 6,500 nt, about 4,500 nt to about 7,000 nt, about 4,500 nt to about 7,500 nt, about 4,500 nt to about 8,000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 6,000 nt to about 6,500 nt, about 6,000 nt to about 7,000 nt, about 6,000 nt to about 7,500 nt, about 6,000 nt to about 8,000 nt, about 6,500 nt to about 7,000 nt, about 6,500 nt to about 7,500 nt, about 6,500 nt to about 8,000 nt, about 7,000 nt to about 7,500 nt, about 7,000 nt to about 8,000 nt, or about 7,500 nt to about 8,000 nt, the total target protein coding sequence is about 2,000 nt, about 3,000 nt, about 3,500 nt, about 4,000 nt, about 4,500 nt, about 5,000 nt, about 5,500 nt, about 6,000 nt, about 6,500 nt, about 7,000 nt, about 7,500 nt, or about 8,000 nt; and/or
the summed size of the two RNA molecules is about 5,000 nt to about 9000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,000 nt to about 8,500 nt, about 5,000 nt to about 9,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 5,500 nt to about 8,500 nt, about 5,500 nt to about 9,000 nt, about 6,000 nt to about 6,500 nt, about 6,000 nt to about 7,000 nt, about 6,000 nt to about 7,500 nt, about 6,000 nt to about 8,000 nt, about 6,000 nt to about 8,500 nt, about 6,000 nt to about 9,000 nt, about 6,500 nt to about 7,000 nt, about 6,500 nt to about 7,500 nt, about 6,500 nt to about 8,000 nt, about 6,500 nt to about 8,500 nt, about 6,500 nt to about 9,000 nt, about 7,000 nt to about 7,500 nt, about 7,000 nt to about 8,000 nt, about 7,000 nt to about 8,500 nt, about 7,000 nt to about 9,000 nt, about 7,500 nt to about 8,000 nt, about 7,500 nt to about 8,500 nt, about 7,500 nt to about 9,000 nt, about 8,000 nt to about 8,500 nt, about 8,000 nt to about 9,000 nt, about 8,500 nt to about 9,000 nt, about 5,000 nt, about 5,500 nt, about 6,000 nt, about 6,500 nt, about 7,000 nt, about 7,500 nt, about 8,000 nt, about 8,500 nt, or about 9,000 nt.

59. The composition of any one of embodiments 2 and 4-24, wherein:
the total target protein coding sequence size is about 3000 nt to about 12,000 nt, about 3,000 nt to about 4,000 nt, about 3,000 nt to about 5,000 nt, about 3.000 nt to about 6,000 nt, about 3,000 nt to about 7,000 nt, about 3,000 nt to about 7,500 nt, about 3,000 nt to about 8,000 nt, about 3,000 nt to about 8,500 nt, about 3,000 nt to about 9,000 nt, about 3,000 nt to about 1,000 nt, about 3,000 nt to about 11,000 nt, about 3,000 nt to about 12,000 nt, about 4,000 nt to about 5,000 nt, about 4,000 nt to about 6,000 nt, about 4,000 nt to about 7,000 nt, about 4,000 nt to about 7,500 nt, about 4,000 nt to about 8,000 nt, about 4,000 nt to about 8,500 nt, about 4,000 nt to about 9,000 nt, about 4,000 nt to about 1,000 nt, about 4,000 nt to about 11,000 nt, about 4,000 nt to about 12,000 nt, about 5,000 nt to about 6,000 nt, about 5,000 nt to about 7,000 nt, about 5,000 nt to about 7,500 nt, about 5,000 nt to about 8,000 nt, about 5,000 nt to about 8,500 nt, about 5,000 nt to about 9,000 nt, about 5,000 nt to about 1,000 nt, about 5,000 nt to about 11,000 nt, about 5,000 nt to about 12,000 nt, about 6,000 nt to about 7,000 nt, about 6,000 nt to about 7,500 nt, about 6,000 nt to about 8,000 nt, about 6,000 nt to about 8,500 nt, about 6,000 nt to about 9,000 nt, about 6,000 nt to about 1,000 nt, about 6,000 nt to about 11,000 nt, about 6,000 nt to about 12,000 nt, about 7,000 nt to about 7,500 nt, about 7,000 nt to about 8,000 nt, about 7,000 nt to about 8,500 nt, about 7,000 nt to about 9,000 nt, about 7,000 nt to about 1,000 nt, about 7,000 nt to about 11,000 nt, about 7,000 nt to about 12,000 nt, about 7,500 nt to about 8,000 nt, about 7,500 nt to about 8,500 nt, about 7,500 nt to about 9,000 nt, about 7,500 nt to about 1,000 nt, about 7,500 nt to about 11,000 nt, about 7,500 nt to about 12,000 nt, about 8,000 nt to about 8,500 nt, about 8,000 nt to about 9,000 nt, about 8,000 nt to about 1,000 nt, about 8,000 nt to about 11,000 nt, about 8,000 nt to about 12,000 nt, about 8,500 nt to about 9,000 nt, about 8,500 nt to about 1,000 nt, about 8,500 nt to about 11,000 nt, about 8,500 nt to about 12,000 nt, about 9,000 nt to about 1,000 nt, about 9,000 nt to about 11,000 nt, about 9,000 nt to about 12,000 nt, about 1,000 nt to about 11,000 nt, about 1,000 nt to about 12,000 nt, about 11,000 nt to about 12,000 nt, about 3,000 nt, about 4,000 nt, about 5,000 nt, about 6,000 nt, about 7,000 nt, about 7,500 nt, about 8,000 nt, about 8,500 nt, about 9,000 nt, about 1,000 nt, about 11,000 nt, or about 12,000 nt; and/or
the summed size of the three RNA molecules is about 7500 nt to about 13,500 nt, about 7,500 nt to about 8,500 nt, about 7,500 nt to about 9,000 nt, about 7,500 nt to about 9,500 nt, about 7,500 nt to about 10,000 nt, about 7,500 nt to about 10,500 nt, about 7,500 nt to about 11,000 nt, about 7,500 nt to about 11,500 nt, about 7,500 nt to about 12,000 nt, about 7,500 nt to about 12,500 nt, about 7,500 nt to about 13,000 nt, about 7,500 nt to about 13,500 nt, about 8,500 nt to about 9,000 nt, about 8,500 nt to about 9,500 nt, about 8,500 nt to about 10,000 nt, about 8,500 nt to about 10,500 nt, about 8.500 nt to about 11,000 nt, about 8,500 nt to about 11,500 nt, about 8,500 nt to about 12,000 nt, about 8,500 nt to about 12,500 nt, about 8,500 nt to about 13,000 nt, about 8,500 nt to about 13,500 nt, about 9,000 nt to about 9,500 nt, about 9,000 nt to about 10,000 nt, about 9,000 nt to about 10,500 nt, about 9,000 nt to about 11,000 nt, about 9,000 nt to about 11,500 nt, about 9,000 nt to about 12,000 nt, about 9,000 nt to about 12,500 nt, about 9,000 nt to about 13,000 nt, about 9,000 nt to about 13,500 nt, about 9,500 nt to about 10,000 nt, about 9,500 nt to about 10,500 nt, about 9,500 nt to about 11,000 nt, about 9,500 nt to about 11,500 nt, about 9,500 nt to about 12,000 nt, about 9,500 nt to about 12,500 nt, about 9,500 nt to about 13,000 nt, about 9,500 nt to about 13,500 nt, about 10,000 nt to about 10,500 nt, about 10,000 nt to about 11,000 nt, about 10,000 nt to about 11,500 nt, about 10,000 nt to about 12,000 nt, about 10,000 nt to about 12,500 nt, about 10,000 nt to about 13,000 nt, about 10,000 nt to about 13,500 nt, about 10,500 nt to about 11,000 nt, about 10,500 nt to about 11,500 nt, about 10,500 nt to about 12,000 nt, about 10,500 nt to about 12,500 nt, about 10,500 nt to about 13,000 nt, about 10,500 nt to about 13,500 nt, about 11,000 nt to about 11,500 nt, about 11,000 nt to about 12,000 nt, about 11,000 nt to about 12,500 nt, about 11,000 nt to about 13,000 nt, about 11,000 nt to about 13,500 nt, about 11,500 nt to about 12,000 nt, about 11,500 nt to about 12,500 nt, about 11,500 nt to about 13,000 nt, about 11,500 nt to about 13,500 nt, about 12,000 nt to about 12,500 nt, about 12,000 nt to about 13,000 nt, about 12,000 nt to about 13,500 nt, about 12,500 nt to about 13,000 nt, about 12,500 nt to about 13,500 nt, about 13,000 nt to about 13,500 nt about 7,500 nt, about 8,500 nt, about 9,000 nt, about 9,500 nt, about 10,000 nt, about 10,500 nt, about 11,000 nt, about 11,500 nt, about 12,000 nt, about 12,500 nt, about 13,000 nt, or about 13,500 nt.

60. The composition of any one of embodiments 3 and 4-24, wherein:
the total target protein coding sequence size is about 4000 nt to about 16,000 nt, about 5,000 nt to about 6,000 nt, about 5,000 nt to about 7,000 nt, about 5,000 nt to about 8,000 nt, about 5,000 nt to about 9,000 nt, about 5,000 nt to about 10,000 nt, about 5,000 nt to about 11,000 nt, about 5,000 nt to about 12,000 nt, about 5,000 nt to about 13,000 nt, about 5,000 nt to about 14,000 nt, about 5,000 nt to about 15,000 nt, about 5,000 nt to about 16,000 nt, about 6,000 nt to about 7,000 nt, about 6,000 nt to about 8,000 nt, about 6,000 nt to about 9,000 nt, about 6,000 nt to about 10,000 nt, about 6,000 nt to about 11,000 nt, about 6,000 nt to about 12,000 nt, about 6,000 nt to about 13,000 nt, about 6,000 nt to about 14,000 nt, about 6,000 nt to about 15,000 nt, about 6,000 nt to about 16,000 nt, about 7,000 nt to about 8,000 nt, about 7,000 nt to about 9,000 nt, about 7,000 nt to about 10,000 nt, about 7,000 nt to about 11,000 nt, about 7,000 nt to about 12,000 nt, about 7,000 nt to about 13,000 nt, about 7,000 nt to about 14,000 nt, about 7,000 nt to about 15,000 nt, about 7,000 nt to about 16,000 nt, about 8,000 nt to about 9,000 nt, about 8,000 nt to about 10,000 nt, about 8,000 nt to about 11,000 nt, about 8,000 nt to about 12,000 nt, about 8,000 nt to about 13,000 nt, about 8,000 nt to about 14,000 nt, about 8,000 nt to about 15,000 nt, about 8,000 nt to about 16,000 nt, about 9,000 nt to about 10,000 nt, about 9,000 nt to about 11,000 nt, about 9,000 nt to about 12,000 nt, about 9,000 nt to about 13,000 nt, about 9,000 nt to about 14,000 nt, about 9,000 nt to about 15,000 nt, about 9,000 nt to about 16,000 nt, about 10,000 nt to about 11,000 nt, about 10,000 nt to about 12,000 nt, about 10,000 nt to about 13,000 nt, about 10,000 nt to about 14,000 nt, about 10,000 nt to about 15,000 nt, about 10,000 nt to about 16,000 nt, about 11,000 nt to about 12,000 nt, about 11,000 nt to about 13,000 nt, about 11,000 nt to about 14,000 nt, about 11,000 nt to about 15,000 nt, about 11,000 nt to about 16,000 nt, about 12,000 nt to about 13,000 nt, about 12,000 nt to about 14,000 nt, about 12,000 nt to about 15,000 nt, about 12,000 nt to about 16,000 nt, about 13,000 nt to about 14,000 nt, about 13,000 nt to about 15,000 nt, about 13,000 nt to about 16,000 nt, about 14,000 nt to about 15,000 nt, about 14,000 nt to about 16,000 nt, or about 15,000 nt to about 16,000 nt, the total target protein coding sequence is about 5,000 nt, about 6,000 nt, about 7,000 nt, about 8,000 nt, about 9,000 nt, about 10,000 nt, about 11,000 nt, about 12,000 nt, about 13,000 nt, about 14,000 nt, about 15,000 nt, about 16,000 nt about 5,000 nt, about 6,000 nt, about 7,000 nt, about 8,000 nt, about 9,000 nt, about 10,000 nt, about 11,000 nt, about 12,000 nt, about 13,000 nt, about 14,000 nt, or about 15,000 nt; and/or
the summed size of the RNA molecules encoded by the four synthetic DNA molecules is about 10,000 nt to about 18,000 nt, about 10,000 nt to about 11,000 nt, about 10,000 nt to about 12,000 nt, about 10,000 nt to about 13,000 nt, about 10,000 nt to about 14,000 nt, about 10,000 nt to about 15,000 nt, about 10,000 nt to about 16,000 nt, about 10,000 nt to about 17,000 nt, about 10,000 nt to about 18,000 nt, about 11,000 nt to about 12,000 nt, about 11,000 nt to about 13,000 nt, about 11,000 nt to about 14,000 nt, about 11,000 nt to about 15,000 nt, about 11,000 nt to about 16,000 nt, about 11,000 nt to about 17,000 nt, about 11,000 nt to about 18,000 nt, about 12,000 nt to about 13,000 nt, about 12,000 nt to about 14,000 nt, about 12,000 nt to about 15,000 nt, about 12,000 nt to about 16,000 nt, about 12,000 nt to about 17,000 nt, about 12,000 nt to about 18,000 nt, about 13,000 nt to about 14,000 nt, about 13,000 nt to about 15,000 nt, about 13,000 nt to about 16,000 nt, about 13,000 nt to about 17,000 nt, about 13,000 nt to about 18,000 nt, about 14,000 nt to about 15,000 nt, about 14,000 nt to about 16,000 nt, about 14,000 nt to about 17,000 nt, about 14,000 nt to about 18,000 nt, about 15,000 nt to about 16,000 nt, about 15,000 nt to about 17,000 nt, about 15,000 nt to about 18,000 nt, about 16,000 nt to about 17,000 nt, about 16,000 nt to about 18,000 nt, about 17,000 nt to about 18,000 nt, about 10,000 nt, about 11,000 nt, about 12,000 nt, about 13,000 nt, about 14,000 nt, about 15,000 nt, about 16,000 nt, about 17,000 nt, or about 18,000 nt.

61. The system of any one of embodiments 1 to 24 and 57 to 60, wherein the first dimerization domain and the second dimerization domain, the third dimerization domain and the fourth dimerization domain, and/or the fifth dimerization domain and the sixth dimerization domain, are each no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150, 200, 250, 300, 400, or 500 nt; and the system has a recombination efficiency of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

62. The system of any one of embodiments 1 to 24 and 57 to 61, wherein each dimerization domain is no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150, 200, 250, 300, 400, or 500 nt; and the system has a recombination efficiency of at least 20%, at least 30%, at least 40%, at least 50%, at least 60%, at least 70%, at least 75%, at least 80%, or at least 90%.

63. The composition of any one of embodiments 1 to 24 and 57 to 62, wherein the RNA recombination efficiency is about 10% to about 100%, about 10% to about 20%, about 10% to about 30%, about 10% to about 35%, about 10% to about 40%, about 10% to about 45%, about 10% to about 50%, about 10% to about 55%, about 10% to about 60%, about 10% to about 70%, about 10% to about 80%, about 10% to about 90%, about 20% to about 30%, about 20% to about 35%, about 20% to about 40%, about 20% to about 45%, about 20% to about 50%, about 20% to about 55%, about 20% to about 60%, about 20% to about 70%, about 20% to about 80%, about 20% to about 90%, about 30% to about 35%, about 30% to about 40%, about 30% to about 45%, about 30% to about 50%, about 30% to about 55%, about 30% to about 60%, about 30% to about 70%, about 30% to about 80%, about 30% to about 90%, about 35% to about 40%, about 35% to about 45%, about 35% to about 50%, about 35% to about 55%, about 35% to about 60%, about 35% to about 70%, about 35% to about 80%, about 35% to about 90%, about 40% to about 45%, about 40% to about 50%, about 40% to about 55%, about 40% to about 60%, about 40% to about 70%, about 40% to about 80%, about 40% to about 90%, about 45% to about 50%, about 45% to about 55%, about 45% to about 60%, about 45% to about 70%, about 45% to about 80%, about 45% to about 90%, about 50% to about 55%, about 50% to about 60%, about 50% to about 70%, about 50% to about 80%, about 50% to about 90%, about 55% to about 60%, about 55% to about 70%, about 55% to about 80%, about 55% to about 90%, about 60% to about 70%, about 60% to about 80%, about 60% to about 90%, about 70% to about 80%, about 70% to about 90%, about 80% to about 90%, about 10%, about 20%, about 30%, about 35%, about 40%, about 45%, about 50%, about 55%, about 60%, about 70%, about 80%, about 90%, about 95%, or about 100%.

64. The composition of any one of embodiments 25 to 30 and 44 to 45, the system of any one of embodiments 31 to 43, or the method of any one of embodiments 47-50, wherein the synthetic DNA is produced by transcription of an RNA virus genome by reverse transcriptase.

It is understood that any embodiment of the disclosure as described herein may be applied, as appropriate, to the described compositions, systems, methods and kits relating to the expression of a nucleic acid editor protein. The present disclosure includes the use of a composition, system, method or kit as described herein to treat a disease in or otherwise provide benefit to a subject in need thereof, and a composition, system, method or kit as described herein for use in treating a disease in or otherwise provide benefit to a subject in need thereof.

### Example 1

### Synthetic RNA Dimerization and Recombination Domains

FIG. 1A depicts a schematic of vector designs (left) and RNA interactions and splicing (right). Left: 5′ trans-splice (trsp) DNA vector: Open arrows are two opposing promoters. RFP coding domain and 3′UTR with poly adenylation elements are expressed opposite from the N-terminal portion of YFP (n-yfp), followed by a splice donor sequence (SD), a downstream intronic splicing enhancer (DISE), and two intronic splicing enhancers (2×ISE), a binding domain (BD, also referred to as dimerization domain), and a stable stem loop BoxB element (boxB), a self-cleaving hammerhead ribozyme (HHrz), ending with a 3′ UTR containing poly adenylation elements. The n-yfp segment has a small intron inserted (white segment within n-yfp). 3′ trsp DNA vector: Open arrows are two opposing promoters. BFP coding domain and 3′UTR with poly adenylation elements are expressed opposite from complementary binding domain (anti-BD, also referred to as dimerization domain), followed by three intronic splicing enhancer sequences (3×ISE), a branch point (BP), a polypyrimidine tract (PPT), a splice acceptor sequence (SA), the c-terminal proton of the YFP coding sequence, ending with a 3′ UTR containing poly adenylation elements. Right: pre-mRNA interactions (5′ trsp-RNA+3′ trsp-RNA) and trans-splicing to generate an mRNA encoding YFP protein are shown.

FIG. 1B depicts transfection of only the N-terminal expression plasmid does not lead to YFP fluorescence. Flow cytometry displaying 20 k RFP+ cells.

FIG. 1C depicts transfection of only the C-terminal expression plasmid does not lead to YFP fluorescence. Flow cytometry displaying 20 k BFP+ cells.

FIG. 1D depicts expression of N-terminal and C-terminal fragments without binding domains shows low levels of YFP induction. Flow cytometry displaying red and green fluorescence values for 20k BFP+ cells.

FIG. 1E depicts rationally designed dimerization/binding domain in a looped configuration.

Segments of hypodiverse exclusively pyrimidine or exclusively purine containing sequences are interspaced with stable stem sequences. RNA folding predictions shows 6 stretches of open sequence (numbered 1-6) available for base pairing between the binding domain and its complementary sequence.

FIG. 1F depicts 3D rendering of the “looped” dimerization domain configuration showing the 6 stretches of open sequence (numbered 1-6).

FIG. 1G depicts negative control with no binding domain on the C-terminal half. Flow cytometry displaying red and green fluorescence values for 20k BFP+ cells.

FIG. 1H depicts negative control with no binding domain on the N-terminal half. Flow cytometry displaying red and green fluorescence values for 20k BFP+ cells.

FIG. 1I depicts matching binding domains on both N- and C-terminal half shows strong YFP induction in 90% of the cells. Flow cytometry displaying red and green fluorescence values for 20k BFP+ cells.

FIGS. 1J-1N depict data equivalent to that in FIGS. 1E-II for a configuration of a binding domain with a stretch of 150 hypodiverse exclusively pyrimidine or exclusively purine containing sequence resulting in a fully open configuration.

FIG. 1O depicts representative fluorescence images for cells shown in FIG. 1G.

FIG. 1P depicts representative fluorescence images for cells shown in FIG. 1L.

FIG. 1Q depicts a comparison of conditions shown in FIG. 1D, FIGS. 1G-1I, and FIGS. 1L-1N.

YFP induction coefficient is calculated: (#R+Y+÷#R+Y−)×100×med·Y−fluor(R+Y+). For comparison the recombination efficiency of a native intron (intron I of the mouse parvalbumin gene) on the N-terminus and an optimized binding domain for that intron on the C-terminal fragment are shown (white bar). This illustrates the benefits of the optimized synthetic RNA dimerization and recombination domains.

### Example 2

Reconstitution of Protein from Three Synthetic Fragments

FIG. 2A depicts an exemplary schematic of vector designs. The protein coding sequence of a YFP is split into an N-terminal fragment, a middle fragment (m-yfp) and a C-terminal fragment. The junction of the n and m fragments is joined by a looped design binding domain (BD1) and the junction between m and c fragments is joined by a looped binding domain (BD2). The pyrimidine (Y) and purine (R) sequences are arranged to avoid self-circularization of the m-fragment and avoid direct recombination of the N- and C-fragment. The N-terminal fragment is co-expressed with red fluorescent protein as a transfection control, the C-terminal fragment is coexpressed with blue fluorescent protein as a transfection control.

FIG. 2B depicts matching binding domains on all three fragments shows strong YFP induction in 80% of the cells. Flow cytometry displaying red and green fluorescence values for 20k BFP+ cells.

FIG. 2C depicts representative fluorescent image of expression of the n and m fragment only shows no YFP fluorescence (negative control).

FIG. 2D depicts representative fluorescent image of expression of the m and c fragment only shows no YFP fluorescence (negative control).

FIG. 2E depicts representative fluorescent image showing that strong YFP fluorescence is induced by co-transfection of all three fragments.

### Example 3

In Vivo Delivery of Reconstituted Full-Length YFP Divided into Two Portions

Reconstitution of a YFP coding sequence from two fragments is achieved by using two synthetic RNA sequences, wherein one included the n-terminal coding half fragment of YFP, and one included the c-terminal coding half fragment (FIG. 3A) (SEQ ID NOS 1 and 2). Each fragment was expressed from AAV2/8 after systemic (iv) administration in newborn (P3) mouse pups. A total of 1.88E11 viral genomes for each of the two fragments were administered per mouse. Expression of YFP was detected 3 weeks later in the liver, heart muscle, and skeletal muscle using fluorescence microscopy.

As shown in FIG. 3B, expression of full-length YFP was detected in the liver of the juvenile mouse, while uninjected liver showed no YFP expression.

As shown in FIG. 3C, expression of full-length YFP was detected in the heart muscle of the juvenile mouse, while uninjected heart muscle showed no YFP expression.

As shown in FIG. 3D, expression of full-length YFP was detected in the skeletal muscles of the leg, while uninjected liver showed no YFP expression.

Thus, the disclosed systems can be used to express full-length proteins in vivo, from two or more separate synthetic RNA molecules.

### Example 4

In Vivo Delivery of Reconstituted Full-Length YFP Divided into Three Portions

Reconstitution of a YFP coding sequence from three fragments is achieved by using three synthetic RNA sequences, wherein one included the n-terminal fragment of YFP, one included a middle fragment of YFP, and one included the c-terminal fragment (FIG. 4A) (SEQ ID NOS: 145, 146 and 2 respectively).

Each fragment was expressed from AAV2/8 after intramuscular injection into the tibialis anterior muscle of newborn (P3) mouse pups. A total of 1E11 viral genomes for each of the fragments was administered intramuscularly. Expression of YFP was detected 3 weeks later in the skeletal muscle using fluorescence microscopy.

As shown in FIG. 4B, expression of full-length YFP fluorescence was observed in the tibialis anterior muscle.

Thus, the disclosed systems can be used to express full-length proteins in vivo, from three or more separate synthetic RNA molecules.

### Example 5

### In Vivo Delivery of Reconstituted Full-Length Protein

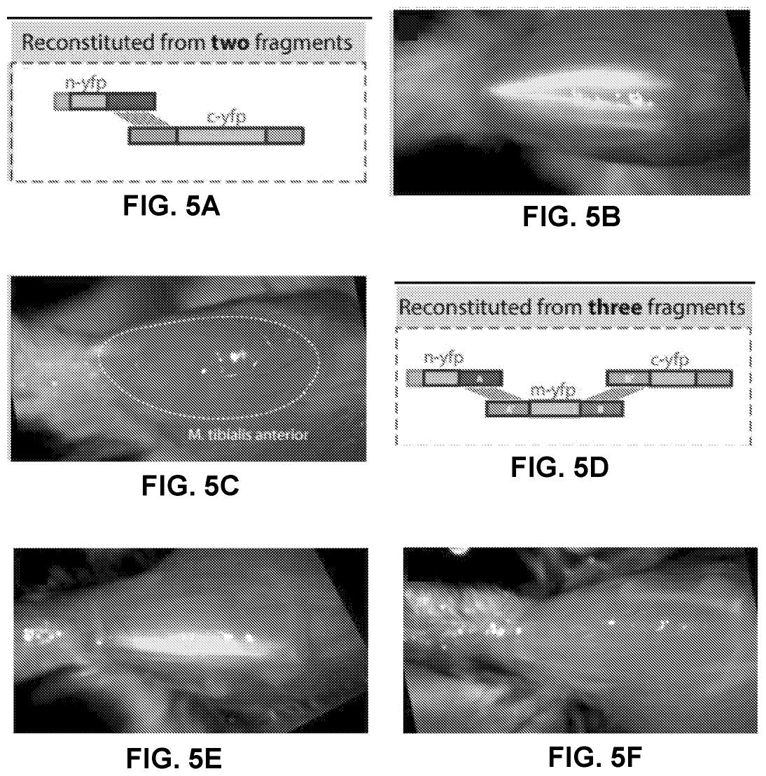

To demonstrate the feasibility of a three-part sRdR system in vivo, a combination of either two or three AAV-transfer plasmids (the DNA precursor plasmids of AAV) containing fragments of the YFP were transcutaneously electroporated into the tibialis anterior (TA) hindlimb muscle of adult mice. Efficient reconstitution of both the two part split-YFP system as well as the three part split-YFP system was observed five days after intramuscular electroporation (FIGS. 5A-5F).

FIGS. 5A-5F depict efficient reconstitution of YFP from two and from three fragments in adult mouse tibialis anterior muscle. FIG. 5A depicts N-terminal and C-terminal halves of YFP coding sequences are equipped with synthetic RNA-dimerization and recombination domains. FIG. 5B depicts two AAV transfer plasmids expressing these two fragments were electroporated transcutaneously into adult mouse tibialis anterior (TA) muscle and strong fluorescence was detected at 5 days post electroporation. FIG. 5C shows no fluorescence was detectable in contralateral non-injected TA. FIG. 5D depicts N-terminal, middle, and C-terminal YFP coding sequence are equipped with synthetic RNA-dimerization and recombination domains linking each fragment to its adjacent fragment(s). FIG. 5E depicts transcutaneous electroporation of three AAV transfer plasmids expressing these three fragments. Strong YFP fluorescence is detected indicating efficient reconstitution of YFP from three fragments. FIG. 5F depicts fluorescence in contralateral non-injected TA. Fluorescent channel is overlaid onto grey scale photographs for context.

Two or three vectors were used to successfully express YFP in liver, cardiac muscle and skeletal muscle (two AAV vectors), and in skeletal muscle (three AAV vectors).

Hence the synthetic RNA-dimerization and recombination system provided herein can be deployed in the muscle. Based on these results, one can substitute the YFP coding sequence with a dystrophin (or other gene) coding sequence to achieve therapeutic full-length dystrophin (or other gene) expression from AAVs into a desired subject and/or tissue.

### Example 6

### Delivery of Reconstituted Full-Length Dystrophin to Treat DMD

An effective gene therapy using full-length dystrophin for patients who suffer from Duchenne muscular dystrophy (DMD) has remained challenging, because the coding sequence of this large protein exceeds the capacity of most viral vectors. Adeno-associated viruses (AAVs) are a common and the preferred method of gene delivery in gene replacement therapy. AAVs are non-toxic, well tolerated, and lead to long term expression of the replacement gene without random integration into the genome. However, the dystrophin gene is too large to be delivered by a single virus. If broken down into fragments, full-length dystrophin can only be delivered using a minimum of three viruses. Smaller versions of dystrophin called “micro-Dystrophin” or “mini-Dystrophin” are currently being tested for dystrophin gene replacement therapy, but these truncated versions of dystrophin are not expected to have full functionality as they are missing key domains in the rod and hinge section of the protein. To date, past attempts to overcome this limitation have not yielded the efficiency required for treating DMD.

Provided herein is a novel technology that can be used to efficiently reconstitute the coding sequence of large genes, including dystrophin, from multiple serial fragments. Using this technology in combination with AAV as a delivery vector, full-length dystrophin will be expressed in a murine model (as well as pig and canine models) for DMD. In one example the subject is a human adult, juvenile, or infant with DMD. For example, the disclosed methods and systems can be used to deliver synthetic RNA-dimerization and recombination domains encoding full-length dystrophin over two or three AAVs (e.g., each AAV delivering a half or a third of the full-length coding sequence). In one example, the AAVs are myotropic AAVs (e.g., those that preferentially infect muscles). This approach can be used to ameliorate or prevent the onset of dystrophy symptoms in a mouse or canine model for DMD, as well as human subjects.

Part 1: Construct efficiently reconstituted three-way split dystrophin expression cassettes. Three expression cassettes are constructed that efficiently reconstitute the full-length dystrophin coding sequence in vitro while each individual expression cassette is within the packaging limit of conventional AAV vectors. To achieve therapeutically effective levels of dystrophin, the expression system can be optimized to achieve roughly physiological levels of dystrophin or moderately supraphysiological levels. Up to 50-fold overexpression of dystrophin is tolerated without adverse effects. The dystrophin coding sequence can be split at a number of different points along its length. Efficiency of reconstitution, however, is affected by the local RNA microenvironment and maximization of reconstitution efficiency is done empirically by comparing efficiency of several possible split points. The natural dystrophin coding sequence can be codon optimized for optimal expression and modified to accommodate maximal reconstitution efficiency. It is expected that the full-length dystrophin coding sequence can be reconstituted from a three-way split precursor using the synthetic RNA-dimerization and recombination approach herein disclosed. In screening different configurations, the set of three expression cassettes that lead to the most efficient reconstitution of dystrophin (e.g., approximately physiological or moderately supraphysiological levels) are selected. Experiments can be performed in HEK293T or Human Skeletal Muscle Cells (HSkMC, either primary or trans-differentiated). Using endogenous vs. exogenous specific quantitative RT-PCR probes, and by epitope tag detection in the exogenous dystrophin protein and Western blot analysis, reconstitution efficiencies will be determined different configurations of the split/reconstituted dystrophin.

Part 2: Maximize full-length dystrophin expression over non-reconstituted fragments. Suppression of fragmented background expression of non-reconstituted dystrophin can be achieved by modification of the synthetic RNA-dimerization and recombination domains. Non-reconstituted fragment expression caused by inefficiencies in RNA-recombination may lead to background expression of dystrophin fragments. Further, suppression of this fragmented background expression may be achieved by modification of the synthetic RNA-dimerization and recombination domains. With the disclosed approach, each fragment of dystrophin is transcribed separately. Reconstitution occurs on the RNA level. Each individual fragment can therefore potentially be translated without being reconstituted. In a western blot, with full-length dystrophin running at roughly 430 kDa, these fragments would run at sizes of about ⅔ (˜290 kDa) and ⅓ (˜140 kDa) of that. The synthetic RNA-dimerization and recombination domains can be optimized to avoid non-reconstituted fragment expression and favor full length expression of dystrophin. This can for example be achieved by strategically placing degron sequences, disrupting RNA nuclear export of non-recombined fragments, and introducing decoy translation initiation points. Experiments are carried out in HEK293T and HSkMC. The dystrophin coding sequence can be bookended with epitope tags that allow for identification and quantification of not fully reconstituted fragments of dystrophin using western blot analysis. Cellular distribution of these dystrophin fragments will be assessed using immunohistochemistry in skeletal human muscle cells. Additionally, quantitative assessment of fragment suppression will be done using conventional molecular biology techniques, including quantitative RT PCR across the recombination junctions will be used to determine how efficient the reconstitution on an RNA level occurs. It is expected that low levels of fragmented dystrophin expression will be observed. By modifying the synthetic RNA-dimerization and recombination domains, these fragments can be suppressed.

Part 3. Create high-titer AAV stocks of full-length dystrophin modules for in vitro and in vivo expression. Dystrophin expressing AAVs will be produced with high purity and viral genome counts higher than 3E13 GC/m1. Three myotropic AAV serotypes will be produced: AAV2/8, AAV2/9, and AAV2/rh10. A tripartite split fluorescent protein, a tripartite split of a full-length dystrophin bookended with epitope tags (see Part 2 above), and a non-tagged tripartite split of full-length dystrophin will be produced, resulting in 27 high-titer AAV preparations. Systemic delivery of therapeutic AAV particles requires high concentration large virus preparations. To achieve reconstituted expression of dystrophin form three separate viruses, repeated administration of the virus may be performed. AAV production in HEK293T cells. Iodixanol or CsCl purification. All batches will be tested in vitro in HEK293T and human skeletal muscle cells. As outlined in Part 1 and 2, reconstitution efficiency and unwanted fragment expression will be assessed.

Part 4. Measure expression/reconstitution levels of FLD-AAV modules in vivo and tissue distribution in vivo of full-length dystrophin expressing AAV modules. The same are assessed for a tripartite split fluorescent protein, as surrogate indicator. For in vivo delivery, direct intramuscular (cardiac and skeletal muscles) and systemic intravenous delivery in newborn and juvenile mice will be compared. Direct muscle injection of FLD-AAV may result in efficient expression of full-length dystrophin as indicated in the Examples above. Systemic delivery of FLD-AAV will be examined using immunohistochemistry and western blot analysis. Different routes of administration, including direct intramuscular and systemic intravenous delivery, in newborn and juvenile mice will be compared. The analysis will focus on: (1) skeletal muscles (major forelimb, hindlimb, shoulder, abdominal and, face muscles) and differential infectivity of fast vs. slow twitch muscles, will be assessed by comparing tibialis anterior and soleus muscles, (2) cardiac muscle expression, and (3) liver expression. This cohort of animals will be monitored for possible adverse effects of the high-titer AAV injections.

Although direct muscular injection of AAVs represents an approach to delivering the FLD-AAV modules (which in light of the results in FIGS. 5A-5F is likely to be successful), it is nonetheless desirable from a clinical perspective to achieve full-length dystrophin expression using systemic i.v. delivery of the virus. In vitro FLD-AAV testing will be used to determine how AAV copy number and reconstituted dystrophin levels correlate. Tissue distribution and efficiency of reconstitution will be assessed in vivo, and different delivery paradigms (e.g., serotype, viral titer, route of application, number of repeat applications) will be examined to achieve optimal tissue distribution. Tissue coverage and expression levels will be assessed. Beneficial outcomes can be achieved even if only a portion of muscle fibers express dystrophin (e.g., normal heart function with only about 50% of cardiomyocytes being dystrophin deficient under non-stress conditions). Both, physiological and supraphysiological levels of dystrophin are of therapeutic value. Quantitative assessment will be performed as outlined in Part 1 & 2. In vivo intramuscular and systemic virus application will be performed in neonatal or juvenile mice under aseptic condition.

Part 5. Treat DMD mouse model (mdx) with FLD-AAV and assess disease onset/progression. FLD-AAV delivery in neonatal mdx mice may prevent the onset and progression of myopathy and cardiomyopathy. After optimization of the viral delivery of reconstituted full-length dystrophin (Parts 1-4) FLD-AAV treatment will be administered to a mouse model of DMD. These mice, depending on the genetic background they are bred, present with myopathy that is notably less pronounced than human DMD. Mice with the genetic background that presents with a more severe phenotype (D2.B10-Dmdmdx) show increased hind-limb weakness, lower muscle weight, fewer myofibers, and increased fat and fibrosis. These parameters can be compared between wild-type controls, treated mdx, and untreated mdx mice. The desired outcome is an amelioration or prevention of disease onset/progression.

Two mouse lines, C57BL/10ScSn-Dmdmdx/J, and D2.B10-Dmdmdx/J, which carry a mutation in the dystrophin gene are used. FLD-AAV is delivered according to parameters established as described under Part 4. Animals are injected in the first postnatal week, in a time window before onset of myonecrosis in mdx mice. Wild-type, treated-mdx and vehicle/sham-treated-mdx mice are e assessed for behavioral and anatomical signs of skeletal and cardiac myopathy. Using kinematic and electromyographic testing equipment, performance of these mice in a variety of motor tasks is assessed, such as balance beam, grip strength, horizontal ladder, treadmill speed challenge, over ground locomotor kinematic assessment, and swimming kinematic assessment (ambient temperature and cold water challenge). It will be determined whether FLD-AAV therapy can prevent the presentation of cardiomyopathy in mdx mice following chemical challenge.

The desired outcome of these experiments would be an amelioration or prevention of disease onset/progression.

### Example 7

### Delivery of Reconstituted Full-Length MYO7A Treat Usher Syndrome

A first half of the MYO7A coding sequence is appended with a synthetic RNA dimerization and recombination domain and expressed from a first vector/plasmid. The second half of MYO7A is appended to the complementary RNA dimerization and recombination domain and expressed from a second vector/plasmid. If expressed together in the same cell the two halves of MYO7A are recombined to form the full-length MYO7A transcript which is then translated into protein.

### Example 8

### Transcriptional/Expressional Logic Gate

Breaking a target gene into two nonfunctional halves that get expressed from either two different promoters or using two different delivery vehicles can result in an intersectional expression pattern.

For example, promoter 1 of a first synthetic nucleic acid molecule provided herein can drive expression of the N-terminal half of the coding sequence in for example cell types A, B, and C, while promoter 2 of a second synthetic nucleic acid molecule provided herein drives expression of the C-terminal half in a subset of cells A, D, E, and F. In such an example, the effector gene encoding the target protein is only expressed in the overlapping area (in this example in cell population A).

A similar intersectionality can be used by making the two halves conditionally expressed, for example, under the condition of the presence of a recombinase. Another level at which intersectionality can be achieved is by delivering the two halves with two viruses that have different tropisms.

### Example 9

### Complementation

The disclosed methods and systems can be used to make any gene (and corresponding target protein) into complementation parts (similar to the principle of alpha complementation of LacZ), by encoding two non-functional halves on separate plasmids that only become active when both plasmids are present.

### Example 10

### Trigger RNA

The disclosed systems and methods can be configured such that reconstitution of the two or more portions of the coding sequences of the target protein depends on the presence of a specific “trigger” RNA molecule. As shown in FIG. 7B, in this example, the dimerization domains of each synthetic nucleic acid molecule are not reverse complements of one another, but instead specifically hybridize to adjacent regions of a third RNA molecule, a “trigger RNA”, which serves as a bridge to bring two synthetic nucleic acid molecules together. In this example, the system can “report” the presence of a specific RNA molecule which allows for “cell type specific triggering” of a reporter/effector protein.

### Example 11

### Inclusion of Stabilizing Element in 3′-UTR

This example describes methods used to evaluate recombination of split coding sequences in the presence of a sequence in the 3′-UTR that stabilizes RNA. Woodchuck hepatitis posttranscriptional regulatory element 3 (WPRE3) was used as an exemplary stabilizing sequence. One skilled in the art will appreciate that other RNA sequence stabilizers can be used in place of WPRE3.

Median YFP fluorescence was measured by flow-cytometry for a two-way split YFP that is reconstituted using the disclosed synthetic RNA dimerization and recombination approach. The C-terminal YFP coding fragment is followed by a poly adenylation signal only (w/o WPRE3) or by a truncated version of the woodchuck hepatitis posttranscriptional regulatory element, WPRE3 followed by a poly adenylation signal (labelled w/WPRE3). The N-terminal YFP coding fragment is coexpressed with a red fluorescent protein from a bidirectional promoter for transfection control. The C-terminal fragment is co-expressed with a blue fluorescent protein from a bidirectional promoter as transfection control. Cells with equal red and blue fluorescent control values between conditions are compared.

As shown in FIG. 8, inclusion of a stabilizing element in the 3′-UTR increased expression efficiency of the recombined full-length YFP by about 50-60%. This enhancement is observed even though WPRE sequences stimulate nuclear export of the RNA molecule they are contained in, which may have negatively impacted the RNA joining reaction (and thus gene expression) by shuttling molecule 150 of FIG. 6A outside the nucleus before the spliceosome mediated RNA joining can occur and thus rendering it non-functional.

Thus, the disclosed synthetic molecules (such as any of SEQ ID NOS: 159, 160, 161, 162, 163, 164, 165, and 166) can be modified to further include a RNA sequence stabilizer.

### Example 12

### Effect of Binding Domain Length on Reconstitution Efficiency

Binding domain length was assessed as follows. YFP was split into two non-fluorescent halves (SEQ ID NOS: 1 and 2, but with different length binding domains). Reconstitution efficiency for different length binding domains (ranging from 50 to 500 nucleotides) was assessed in cultured HEK 293t cells. N-terminal YFP is expressed from a bidirectional CMV promoter with a Red Fluorescent Protein (RFP) as a transfection control. C-terminal YFP is expressed from a bidirectional CMV promoter with a Blue Fluorescent Protein (BFP) as a transfection control. For the different binding domain lengths, YFP median fluorescence intensity was compared. Cells with matching RFP and BFP transfection levels are compared between conditions.

As shown in FIG. 11, all of the molecules achieved some level of expression of the full-length YFP, with varying degrees of reconstitution efficiency. Although maximal performance was observed with binding domain lengths of 150 bp and below (e.g. 50-150 bp), binding domain lengths of up to 500 bp were still able to recombine and express full-length YFP.

### Example 13

### Effect of Splicing Enhancer Sequences

This example describes methods used to assess the effect of including one or more intronic splicing enhancer sequences (e.g., 118, 120, 156 in FIG. 6A) in the disclosed synthetic introns.

YFP was split into two non-fluorescent halves (FIG. 12A). Reconstitution efficiency for different intron configurations was assessed in cultured HEK 293t cells. N-terminal YFP was expressed from a bidirectional CMV promoter with a Red Fluorescent Protein (RFP) as a transfection control. C-terminal YFP was expressed from a bidirectional CMV promoter with a Blue Fluorescent Protein (BFP) as a transfection control. For the different intron configurations, YFP median fluorescence intensity is compared. Cells with matching RFP and BFP transfection levels are compared between conditions.

As shown in FIG. 12A, the 5′ molecule (SEQ ID NO: 1) includes the coding region of the N-terminal portion of YFP (n-yfp), followed by a splice donor sequence (SD), a downstream intronic splicing enhancer (DISE), and two intronic splicing enhancers (2×ISE), a binding domain (BD), a self-cleaving hammerhead ribozyme (HHrz), ending with a poly adenylation signal (pA). The 3′ molecule (SEQ ID NO: 2) includes the complementary binding domain (anti-BD), followed by three intronic splicing enhancer sequences (3×ISE), a branch point (BP), a polypyrimidine tract (PPT), a splice acceptor sequence (SA), the c-terminal proton of the YFP coding sequence, ending with a poly adenylation signal (pA).

As shown in FIG. 12B, inclusion of splice enhancers to both the 5′ and the 3′ molecules increases reconstitution efficiency of the full-length YFP. Removal of the splice enhancers reduces the reconstitution efficiency of the two coding sequences by about 50-90%. In the first column, YFP is reconstituted using the reference configuration (SEQ ID NOS: 1 and 2), the second column shows the reconstitution efficiency with deletion of the ISE elements in the 5′ fragment, the third column shows reconstitution efficiency after deletion of the ISE and the DISE in the 5′ fragment. The fourth column shows the reconstitution efficiency after deletion of the HHrz in the 5′ fragment. The fifth column shows reconstitution efficiency using the reference configuration. The sixth column shows reconstitution efficiency after deletion of the ISE elements in the 3′ fragment. The seventh shows reconstitution efficiency after deletion of the ISE in both 5′ and 3′ fragment and the DISE in the 5′ fragment.

### Example 14

### Dual Projection Tracing

This example describes methods used to perform dual projection tracing by reconstitution of full-length flp recombinase (Flpo) from two fragments (SEQ ID NOS: 147 and 148). As shown in FIG. 13A, the Flp recombinase gene was split into two non-functional halves. The N-terminal half of the Flpo gene was joined at its 3′ end with a synthetic intron sequence followed by a dimerization domain sequence (RNA end joining module, REJ). The C-terminal half of the Flpo gene was joined at its 5′ end with a synthetic intron and a dimerization domain (REJ-module). Upon infection of a cell with both constructs, and expression of pre-mRNA from each construct, the pre-mRNAs bound at the dimerization domains (shown by dark parallel bars in FIG. 13A), and the resulting complex was spliced to produce the full length Flpo recombinase mRNA transcript. Thus, the functional recombinase protein was produced by reconstitution of the two fragments. FIG. 13B shows a schematic of a flp activity reporter mouse carrying a flpo dependent red fluorescent protein (tdTomato) (Rosa-CAG-frt-STOP-frt-tdTomato). The two synthetic nucleic acid (DNA) constructs were packaged into separate adeno-associated viruses (retrogradely transported serotype AAV2/retro). AAV2/retro-n-flpo, virus carrying the first construct, was injected in the left primary motor cortex of the mouse, and AAV2/retro-c-flpo, virus carrying the second construct, was injected in the right primary motor cortex of the mouse.

As shown in FIGS. 13C-13D, primary motor cortex cells with axons that cross the midline are labeled with the red fluorescent protein (and appear white in FIGS. 13C and 13D). Hoechst staining (nuclei) is shown for context.

### Example 15

### Expression of Long Protein In Vivo

This example describes methods used to achieve efficient expression of oversized cargo in cell culture and in vivo in the mouse primary motor cortex.

To simulate a large disease-causing gene that fills up the adeno-associated virus (AAV) cargo capacity of two viruses (i.e., it exceeds single AAV packaging capacity), a split YFP coding sequence was embedded inside a large uninterrupted open reading frame. N-terminally (i.e., on the 5′ side) the first part of the YFP coding sequence is flanked with long stuffer sequences (i.e., an uninterrupted open reading frame) followed by a sequence encoding a 2A self-cleaving peptide. On the C-terminus (i.e., 3′ side) the second part of the YFP coding sequence is followed by a 2A self-cleaving peptide coding sequence and then followed by a long stuffer sequence (i.e., and uninterrupted open reading frame) (FIG. 14A). The first and second synthetic DNA molecules encoding pre-mRNA molecules are shown in SEQ ID NOS: 22 and 23, excluding promoter sequences. The resulting RNA molecules expressed are each about 4000 nt between the transcriptional start site at position 1 of SEQ ID NO: 22 and the transcriptional start site at position 1 of SEQ ID NO: 23 and the polyA tail. The resulting transcribed pre-mRNA molecule (5′ fragment; transcribed from SEQ ID NO: 22) contains a stuffer open reading frame which is followed by a self-cleaving 2A peptide encoding sequence, followed by a sequence encoding the N-terminal portion of YFP, followed by a synthetic intron and a dimerization domain (having kissing loop architecture), and a polyA tail. The C-terminal pre-mRNA molecule (3′ fragment; transcribed from SEQ ID NO: 23) is composed of a complementary kissing loop dimerization domain, a synthetic intron sequence, followed by the C-terminal YFP coding sequence, followed by a self-cleaving 2A peptide coding sequence, followed by a stuffer open reading frame, followed by a polyA tail.

Following production of the pre-mRNA molecules, the dimerization domains bind, and splicing joins the pre-mRNAs to produce a full-length mRNA. During translation, the 2A cleavage sequences flanking the YFP result in the cleaving off of the N and C-terminal stuffer sequences and the production of functional YFP protein.

To determine reconstitution efficiency on an RNA level, two probe based (5′-hydrolysis) quantitative real-time PCR assays are used. The first assay spans a sequence fully contained in the 3′ exonic YFP sequence (labelled 3′ probe). The second assay spans the junction between the 5′ and the 3′ exonic YFP sequence (labelled junction probe). Reconstitution efficiency is calculated as the ratio of (junction probe count)/(3′ probe count).

Quantitative real-time PCR analysis of reconstitution efficiency of the oversize YFP constructs in HEK 293t cells was performed. Full-length oversized YFP is used as reference. The full-length oversized YFP ratio is set to 1 (FIG. 14B). Ratio of reconstituted is expressed as fraction of full-length (labelled split-REJ (split RNA end joining)). Reconstitution efficiency is calculated as follows: junction/3′prime. As shown in FIG. 14B about 60% of the RNAs joined in the split-REJ system.

Reconstituted YFP protein expression from full-length oversized YFP expression and split-REJ expression is assessed by flow cytometry of transiently transfected HEK 293t cells. As shown in FIG. 14C, the split REJ system achieved about a 45% joining efficiency, even with the large cargo.

in vivo analysis of reconstitution of the large YFP protein was performed as follows. 60nl of adeno-associated virus 2/8, containing 3E9 vg/injection/fragment, was injected into the primary motor cortex of the mouse. Tissue was harvested 10 days post injection. As shown in FIG. 14D, YFP fluorescence is readily detectable in the bulk tissue (top left, top middle panel, macroscopic top view of the mouse brain, YFP fluorescence plus auto-fluorescence for context are shown). Strong YFP signal is detected at and around the virus injection site in layer 5 of the motor cortex (right panel, cortical layers are numbered 1 to 6, approximate injection depth is indicated by gray bar, scale bar=100 micrometers). Thus, the disclosed system can be used to express large proteins in vivo.

### Example 16

### Expression of Factor VIII

This example describes methods used to achieve efficient reconstitution of full-length human coagulation factor VIII (FVIII).

A schematic of the 5′ and 3′ nucleic acid molecules used for the experiment is shown in FIG. 15A (DNA encoding the pre-RNA molecules are set forth in SEQ ID NOS: 24 and 25, respectively). Each half includes about 3.8 kb of FVIII coding sequence. The resulting RNA 5′-sequence containing the N-terminal half (e.g., as shown schematically in 110 of FIG. 6A) of the FVIII coding sequence is followed by an efficient synthetic intron and a dimerization domain (kissing loop architecture), and a polyA tail. The 3′-sequence containing the C-terminal half (e.g., 150 of FIG. 6A) of the FVIII coding sequence is preceded by the complementary kissing loop dimerization domain, and an efficient synthetic intron sequence. To determine reconstitution efficiency on an RNA level, two probe based (5′-hydrolysis) quantitative real-time PCR assays are used. The first assay spans a sequence fully contained in the 3′ exonic FVIII sequence (labelled 3′ probe). The second assay spans the junction between the 5′ and the 3′ exonic FVIII sequence (labelled junction probe). Reconstitution efficiency is calculated as the ratio of (junction probe count)/(3′ probe count).

PCR quantification of reconstitution efficiency after two days of expression in HEK 293t cells was performed. Full-length FVIII is used as reference. Full-length FVIII ratio is set to one. Reconstituted FVIII assay ratios are expressed as fraction of full-length (labelled split-REJ). As shown in FIG. 15B, a reconstitution efficiency of about 40-60% was achieved (that is about 40-60% of the two RNAs joined in the split-REJ system).

To demonstrate expression of FVIII in vitro, Western blotting was used. FVIII was tagged with an HA-tag at the N-terminus. Constructs are expressed in HEK 293t cells for 2 days. As shown in FIG. 15C, the disclosed split-REJ system successfully expressed full-length FVIII in vitro.

Based on these observations, expression of a full-length FVIII protein in vivo can be achieved, for example to treat hemophilia A. For example, a first half of a FVIII coding sequence is appended with a synthetic RNA dimerization and recombination domain and expressed from a first vector/plasmid. The second half of FVIII is appended to the complementary RNA dimerization and recombination domain and expressed from a second vector/plasmid. If expressed together in the same cell the two halves of FVIII are recombined to form the full-length FVIII transcript which is then translated into protein. For example, a sequence having at least 80%, at least 90%, at least 95%, at least 96%, at least 97%, at least 98%, at least 99% or 100% sequence identity to SEQ ID NO: 24, which includes an N-terminal FVIII coding sequence, and SEQ ID NO: 25 which includes a C-terminal FVII coding sequence, can be utilized for in vivo expression.

### Example 17

### Expression of Abca4

This example describes methods used to achieve efficient reconstitution of full-length human ATP binding cassette subfamily A member 4 (Abca4).

A schematic of the 5′ and 3′ molecules used are shown in FIG. 16A (DNA encoding the pre-RNA molecules are set forth in SEQ ID NOS: 20 and 21, respectively). The 5′ half includes about 3.6 kb of Abca4 coding sequence, and the 3′ half about 3.2 kb of the Abca4 coding region plus a C-terminal 3×FLAG tag. The 5′-sequence contains the N-terminal half of the coding sequence followed by the an efficient synthetic intron sequence and the first dimerization domain (kissing loop). The 3′-sequence containing the C-terminal half of the coding sequence is preceded by the complementary (kissing loop) dimerization domain and an efficient synthetic intron sequence. A Sanger sequencing trace across the junction is shown.

As shown in FIG. 16B, PCR amplification of the junction demonstrates faithful joining of the two coding sequences. To determine reconstitution efficiency on an RNA level, two probe based (5′-hydrolysis) quantitative real-time PCR assays are used (FIG. 16C). The first assay spans a sequence fully contained in the 3′ exonic Abca4 sequence (labelled 3′ probe). The second assay spans the junction between the 5′ and the 3′ exonic Abca4 sequence (labelled junction probe). Reconstitution efficiency is calculated as the ratio of (junction probe count)/(3′ probe count). PCR quantification of reconstitution efficiency after two days of expression in HEK 293t cells is shown in FIG. 16D. Full-length Abca4 is used as reference. Average full-length Abca4 ratio is set to one. Reconstituted Abca4 assay ratios are expressed as fraction of full-length (labelled split-REJ). As shown in FIG. 16D, a reconstitution efficiency of about 35% was achieved (that is about 30-40% of the two RNAs joined in the split-REJ system).

To demonstrate expression of Abca4 in vitro, Western blotting was used. Abca4 is tagged with a 3×FLAG-tag at the C-terminus. Constructs are expressed in HEK 293t cells for 2 days. As shown in FIG. 16E, the disclosed split-REJ system successfully expressed full-length Abca4 in vitro.

Quantification of the western blot is shown in FIG. 16F. To normalize for differential transfection efficiency between conditions, the full-length plasmid and the C-terminal plasmid co-express a Blue Fluorescent Protein for transfection control. BFP concentration in each sample was determined by dot blot and used to normalize between conditions. As shown in FIG. 16F reconstituted Abca4 is expressed at approximately 40% of the levels when compared with direct full-length expression. Hence, the protein levels as determined by western blot, track well with the RNA reconstitution efficiency determined by qPCR.

Based on these observations, expression of a full-length ABCA4 protein in vivo can be achieved, for example to treat Stargardt's Disease. For example, a first half of the ABCA4 coding sequence is appended with a synthetic RNA dimerization and recombination domain and expressed from a first vector/plasmid.

The second half of ABCA4 is appended to the complementary RNA dimerization and recombination domain and expressed from a second vector/plasmid. If expressed together in the same cell the two halves of ABCA4 are recombined to form the full-length ABCA4 transcript which is then translated into protein. For example, a sequence having at least 80%, at least 90%, at least 95%, at least 96%, at least 97%, at least 98%, at least 99% or 100% sequence identity to SEQ ID NO: 20 (FIGS. 10R-10U), which includes an N-terminal Abca4 coding sequence, and SEQ ID NO: 21 (FIGS. 10V-10Z) which includes a C-terminal Abca4 coding sequence, can be utilized for in vivo expression.

### Example 18

### Expression of Otof

This example describes methods used to achieve efficient reconstitution of full-length murine Otoferlin (Otof).

The sequences of the 5′ and 3′ DNA molecules used are shown in SEQ ID NOS: 155 and 156, respectively. The 5′ half includes about 3.5 kb of Otof coding sequence, the 3′ half about 2.5 kb of the Otof coding region plus a C-terminal 3×FLAG tag. The 3′-sequence containing the C-terminal half (e.g., 150 of FIG. 6A) is preceded by the complementary binding domain and an efficient synthetic intron sequence. One skilled in the art will appreciate that a human OTOF coding sequence (e.g., GenBank Accession No. NM_001287489.2 or NM_194248.3) can be substituted for the mouse coding sequence in SEQ ID NOS: 155 and 156.

To demonstrate expression of Otof in vitro, Western blotting was used. Otof is tagged with a 3×FLAG-tag at the C-terminus for Western blot detection. Constructs are expressed in HEK 293t cells for 2 days. As shown in FIG. 18A, the disclosed split-REJ system successfully expressed full-length Otof in vitro.

Quantification of the western blot is shown in FIGS. 18B-C. Raw quantification is shown in the left bar plot (FIG. 18B) as fraction of full-length control. To normalize for differential transfection efficiency between conditions, the full-length plasmid and the C-terminal plasmid co-express a Blue Fluorescent Protein for transfection control. BFP concentration in each sample was determined by confocal fluorescence microscopy prior to harvesting the cells and used to normalize between conditions. Normalized quantification is shown in the right bar plot (FIG. 18C) as normalized fraction of the full-length control. As shown in FIG. 18C reconstituted Otof is expressed at approximately 30% of the levels when compared with direct full-length expression.

Based on these observations, expression of a full-length OTOF protein in vivo can be achieved, for example to treat autosomal recessive deafness 9. For example, a first half of the OTOF coding sequence is appended with a synthetic RNA dimerization and recombination domain (that is an intron and binding domain) and expressed from a first vector/plasmid. The second half of OTOF is appended to the complementary RNA dimerization and recombination domain and expressed from a second vector/plasmid. If expressed together in the same cell the two RNA molecules are expressed in a target cell and the two halves of OTOF coding transcript are recombined to form the full-length OTOF transcript which is then translated into protein. For example, a sequence having at least 80%, at least 90%, at least 95%, at least 96%, at least 97%, at least 98%, at least 99% or 100% sequence identity to SEQ ID NO: 155, which includes an N-terminal Otof coding sequence, and SEQ ID NO: 156 which includes a C-terminal Otof coding sequence, can be utilized for in vivo expression, for example to treat hearing loss.

### Example 19

### Expression of Myo7a

This example describes methods used to achieve efficient reconstitution of full-length human MYOSIN VIIA (Myo7a).

The sequences of the 5′ and 3′ DNA molecules used are shown in SEQ ID NOS: 157 and 158, respectively. The 5′ half includes about 3.6 kb of Myo7a coding sequence, the 3′ half about 3.1 kb of the Myo7a coding region plus a C-terminal 3×FLAG tag. The 3′-sequence containing the C-terminal half (e.g., 150 of FIG. 6A) is preceded by the complementary binding domain and an efficient synthetic intron sequence.

To demonstrate expression of Myo7a in vitro, Western blotting was used. Myo7a is tagged with a 3×FLAG-tag at the C-terminus for Western blot detection. Constructs are expressed in HEK 293t cells for 2 days. As shown in FIG. 19A, the disclosed split-REJ system successfully expressed full-length Myo7a in vitro.

Quantification of the western blot is shown in FIGS. 19B-19C. Raw quantification is shown in the left bar plot (FIG. 19B) as fraction of full-length control. To normalize for differential transfection efficiency between conditions, the full-length plasmid and the C-terminal plasmid co-express a Blue Fluorescent Protein for transfection control. BFP concentration in each sample was determined by confocal fluorescence microscopy prior to harvesting the cells and used to normalize between conditions. Normalized quantification is shown in the right bar plot (FIG. 19C) as normalized fraction of the full-length control. As shown in FIG. 19C reconstituted Myo7a is expressed at approximately 60% of the levels when compared with direct full-length expression.

Based on these observations, expression of a full-length MYO7A protein in vivo can be achieved, for example to treat Usher syndrome, type 1B. For example, a first half of the MYO7A coding sequence is appended with a synthetic RNA dimerization and recombination domain (that is an intron and binding domain) and expressed from a first vector/plasmid. The second half of MYO7A is appended to the complementary RNA dimerization and recombination domain and expressed from a second vector/plasmid. If expressed together in the same cell the two RNA molecules are expressed in a target cell and the two halves of MYO7A coding transcript are recombined to form the full-length MYO7A transcript which is then translated into protein. For example, a sequence having at least 80%, at least 90%, at least 95%, at least 96%, at least 97%, at least 98%, at least 99% or 100% sequence identity to SEQ ID NO: 157, which includes an N-terminal Myo7a coding sequence, and SEQ ID NO: 158 which includes a C-terminal Myo7a coding sequence, can be utilized for in vivo expression.

### Example 20

Expression of dCas9-VPR in a Two-Part System

This example describes methods used to achieve efficient reconstitution of full-length enzymatically dead Cas9 fused to a VPR transcriptional activator domain (dCas9-VPR).

The sequences of the 5′ and 3′ DNA molecules (first and second DNA molecules, encoding first and second RNA molecules) used are shown in SEQ ID NOS: 159 and 160, respectively. The first DNA molecule includes about 3.3 kb of dCas9-VPR coding sequence (dCas9 N-terminal portion), and the second DNA molecule includes about 2.5 kb of the dCas9-VPR coding sequence (dCas9 C-terminal portion). The RNA encoded by the 5′-sequence (first DNA molecule) encodes the N-terminal portion of dCas9 (e.g., 110 of FIG. 6A), followed by an efficient synthetic intron sequence and the complementary binding domain (first dimerization domain). The RNA encoded by the 3′-sequence (second DNA molecule) encodes the C-terminal portion of dCas9 (e.g., 150 of FIG. 6A), preceded by the complementary binding domain (second dimerization domain) and an efficient synthetic intron sequence.

To demonstrate expression of dCas9-VPR in vitro, Western blotting was used. Constructs were expressed in HEK 293t cells for 2 days. As shown in FIG. 20A, the disclosed split-REJ system (“REJ-dual”) successfully expressed full-length dCas9-VPR in vitro.

Quantification of the western blot is shown in FIGS. 20B-20C. Raw quantification is shown in the left bar plot (FIG. 20B) as fraction of full-length control. The full-length control dCas9-VPR was produced from a single construct comprising the intact gene. To normalize for differential transfection efficiency between conditions, the full-length plasmid and the C-terminal plasmid used co-express a Blue Fluorescent Protein (BFP) for transfection control. BFP concentration in each sample was determined by confocal fluorescence microscopy prior to harvesting the cells and used to normalize between conditions. Normalized quantification is shown in the right bar plot (FIG. 20C) as normalized fraction of the full-length control. As shown in FIG. 20C reconstituted dCas9-VPR is expressed at approximately 35% of the levels when compared with direct full-length expression. When expressed together with a UAS targeting guide RNA in HEK 293t cells (FIG. 20D), both full-length and two-way split reconstituted dCas9-VPR induce yellow fluorescent protein expression from a UAS-YFP plasmid, demonstrating functionality of the reconstituted dCas9-VPR.

Based on these observations, expression of a full-length dCas9-VPR protein in vivo can be achieved, for example to activate or overexpress genes. For example, a first RNA molecule comprises a first portion of the dCAS9-VPR protein coding sequence that is appended to a first synthetic RNA dimerization and recombination domain (that is an intron and binding domain). This molecule is expressed from a first vector/plasmid. A second RNA molecule comprises a second portion of the dCAS9-VPR protein coding sequence appended to the complementary second RNA dimerization and recombination domain, is expressed from a second vector/plasmid. When the two RNA molecules are expressed in the same target cell, the two portions of DCAS9-VPR coding transcript are recombined to form the full-length dCAS9-VPR transcript which is then translated into protein. For example, a sequence having at least 80%, at least 90%, at least 95%, at least 96%, at least 97%, at least 98%, at least 99% or 100% sequence identity to SEQ ID NO: 159, which includes an N-terminal dCas9-VPR coding sequence, and SEQ ID NO: 160 which includes a C-terminal dCas9-VPR coding sequence, can be utilized for in vivo expression.

### Example 21

### Expression of Prime Editor in a Two-Part System

This example describes methods used to achieve efficient reconstitution of full-length humanized Cas9 Prime Editor (Prime Editor).

The sequences of the 5′ and 3′ DNA molecules (first and second DNA molecules, encoding first and second RNA molecules) used are shown in SEQ ID NOS: 161 and 162, respectively. The first DNA molecule includes about 3.3 kb of Prime Editor coding sequence (N-terminal portion), and the second portion includes about 3.0 kb of the Prime Editor coding sequence (C-terminal portion). The RNA encoded by the 5′-sequence (first DNA molecule) encodes the N-terminal portion of Prime Editor (e.g., 110 of FIG. 6A), followed by an efficient synthetic intron sequence and the complementary binding domain (first dimerization domain). The RNA encoded by the 3′-sequence (second DNA molecule) encodes the C-terminal portion of Prime Editor (e.g., 150 of FIG. 6A) preceded by the complementary binding domain (second dimerization domain) and an efficient synthetic intron sequence.

To demonstrate expression of Prime Editor in vitro, western blotting was used. Constructs were expressed in HEK 293t cells for 2 days. As shown in FIG. 21A, the disclosed split-REJ system successfully expressed full-length Prime Editor in vitro.

Quantification of the western blot is shown in FIGS. 21B-21C. Raw quantification is shown in the left bar plot (FIG. 21B) as fraction of full-length control. The full-length control Prime Editor was produced from a single construct comprising the intact gene. To normalize for differential transfection efficiency between conditions, the full-length plasmid and the C-terminal plasmid used co-express a Blue Fluorescent Protein for transfection control. BFP concentration in each sample was determined by confocal fluorescence microscopy prior to harvesting the cells and used to normalize between conditions. Normalized quantification is shown in the right bar plot (FIG. 21C) as normalized fraction of the full-length control. As shown in FIG. 21C reconstituted Prime Editor is expressed at approximately 60% of the levels when compared with direct full-length expression. FIG. 21D shows that targeted G to T transversion mutations could be introduced using the full-length and the two-way split prime editors, demonstrating functionality of the protein produced from the two-way split prime editor constructs.

Based on these observations, expression of a full-length PRIME EDITOR protein in vivo can be achieved, for example to treat genomic point mutations. For example, a first RNA molecule comprises a first portion of the PRIME EDITOR coding sequence that is appended with a first synthetic RNA dimerization and recombination domain (that is an intron and binding domain). This molecule is expressed from a first vector/plasmid. A second RNA molecule comprises a second portion of the PRIME EDITOR protein coding sequence appended to the second complementary RNA dimerization and recombination domain, and expressed from a second vector/plasmid. When the two RNA molecules are expressed in the same target cell the two portions of PRIME EDITOR coding transcript are recombined to form the full-length PRIME EDITOR transcript which is then translated into protein. For example, a sequence having at least 80%, at least 90%, at least 95%, at least %%, at least 97%, at least 98%, at least 99% or 100% sequence identity to SEQ ID NO: 161, which includes an N-terminal Prime Editor coding sequence, and SEQ ID NO: 162 which includes a C-terminal Prime Editor coding sequence, can be utilized for in vivo expression.

### Example 22

### Expression of AncBE4 in a Two-Part System

This example describes methods used to achieve efficient reconstitution of full-length humanized Cas9 Cytosine Base Editor (AncBE4).

The sequences of the 5′ and 3′ DNA molecules (first and second DNA molecules, encoding first and second RNA molecules) used are shown in SEQ ID NOS: 163 and 164, respectively. The first DNA molecule includes about 2.4 kb of AncBE4 coding sequence (AncBE4 N-terminal portion), and the second DNA molecule includes about 3.2 kb of the AncBE4 coding sequence (AncBE4 C-terminal portion). The RNA encoded by the 5′ sequence (first DNA molecule) encodes the N-terminal portion of AncBE4 (e.g., 110 of FIG. 6A), followed by an efficient synthetic intron sequence and the complementary binding domain (first dimerization domain). The RNA encoded by the 3′-sequence (by the second DNA molecule) encodes the C-terminal portion (e.g., 150 of FIG. 6A), preceded by the complementary binding domain and an efficient synthetic intron sequence.

To demonstrate expression of AncBE4 in vitro, Western blotting was used. Constructs were expressed in HEK 293t cells for 2 days. As shown in FIG. 22A, the disclosed split-REJ system successfully expressed full-length AncBE4 in vitro.

Quantification of the western blot is shown in FIG. 22B. Raw quantification is shown in the left bar plot (FIG. 22B) as fraction of full-length control. The full-length control AncBE4 was produced from a single construct comprising the intact gene. As shown in FIG. 22B reconstituted AncBE4 is expressed at approximately 40-50% of the levels when compared with direct full-length expression. FIG. 22C shows that targeted C to T transition mutations could be introduced using the full-length and the two-way split AncBE4s, demonstrating functionality of the two-way split AncBE4 constructs.

Based on these observations, expression of a full-length ANCBE4 protein in vivo can be achieved, for example to treat genomic point mutations. For example, a first RNA molecule comprises a first portion of the ANCBE4 protein coding sequence that is appended to a first synthetic RNA dimerization and recombination domain (that is an intron and binding domain). This molecule is expressed from a first vector/plasmid. A second RNA molecule that comprises a second portion of the ANCBE4 protein coding sequence appended to the complementary RNA dimerization and recombination domain is expressed from a second vector/plasmid. When the two RNA molecules are expressed in the same target cell, the two portions of the ANCBE4 coding transcript are recombined to form the full-length ANCBE4 transcript which is then translated into protein. For example, a sequence having at least 80%, at least 90%, at least 95%, at least 96%, at least 97%, at least 98%, at least 99% or 100% sequence identity to SEQ ID NO: 163, which includes an N-terminal AncBE4 coding sequence, and SEQ ID NO: 164 which includes a C-terminal AncBE4 coding sequence, can be utilized for in vivo expression.

### Example 23

### In Vivo Expression of ABESe and Rescue of a Dystrophin Mutation

This example describes methods used to achieve efficient reconstitution of full-length humanized Cas9 Adenosine Base Editor (ABE8e) in vivo, resulting in editing of the mdx premature stop codon and expression of dystrophin in treated muscle.

The sequences of the 5′ and 3′ DNA molecules used are shown in SEQ ID NOS: 225 and 26, respectively (comprising gRNA expression cassettes as described above). The base editor expression constructs in SEQ ID NOS: 225 and 226 are comprised by SEQ ID NOS: 165 and 166, respectively. The first DNA molecule (in SEQ ID NOS: 225 and 165) includes about 2.4 kb of ABE8e coding sequence (ABE8e N-terminal portion), and the second DNA molecule (in SEQ ID NOS: 226 and 166) includes about 3.2 kb of the ABE8e coding sequence. The RNA encoded by the 5′-sequence (first DNA molecule) encodes the N-terminal portion of ABE8e (e.g., 110 of FIG. 6A). The RNA encoded by the 3′-sequence (second DNA molecule) encodes the C-terminal portion (e.g., 150 of FIG. 6A) and precedes the complementary binding domain (second dimerization domain) and an efficient synthetic intron sequence.

To demonstrate expression of ABE8e in vitro, Western blotting was used. Constructs were expressed in HEK 293t cells for 2 days. As shown in FIG. 23A, the disclosed split-REJ system successfully expressed full-length ABE8e in vitro.

Quantification of the western blot is shown in FIG. 23B. Raw quantification is shown in the left bar plot (FIG. 23B) as fraction of full-length control. The full-length control ABE8e was produced from a single construct comprising the intact gene. As shown in FIG. 23B reconstituted ABE8e is expressed at approximately 70% of the levels when compared with direct full-length expression. FIG. 23C shows that targeted C to T transition mutations could be introduced using the full-length and the two-way split ABE8e constructs, demonstrating functionality of the two-way split ABE8e constructs.

To demonstrate efficient reconstitution and expression of full-length humanized Adenine Base Editor (ABE8e) (1606 aa) to correct a premature stop codon, an example for correcting a premature stop codon in the mdx-4cv mouse model for Duchenne muscular dystrophy is shown in FIG. 23D-G. FIG. 23D shows a schematic drawing of the experiment. The ABE8e base editor is split into two fragments, the coding DNA of which are individually packaged into two separate adeno-associated virus capsids. CRISPR gRNAs are designed to target the locus of the premature stop-codon such that the A to G conversion converts the TAA stop codon into a CAA codon.

To demonstrate functionality of the ABE8e base editor and the designed CRISPR gRNAs, FIG. 23E shows a yellow fluorescent protein (YFP) is split into two coding fragments that are connected with a short stretch of the mdx coding sequence surrounding the premature stop codon. The first half of the YFP is non-fluorescent if the open reading frame is terminated by the premature TAA stop codon. Stop codon correction results in translation of the full YFP sequence which is rendered fluorescent (this construct is referred to as YFP-editing-reporter). The left panel shows absence of YFP fluorescence when HEK293T cells were transfected with the YFP-editing-reporter (which co-expresses a red fluorescent transfection control), the N-terminal ABE8e vector, the C-terminal ABE8e vector, and a non-targeting gRNA. The right panel shows expression of YFP fluorescence in a high percentage of cells that are cotransfected with the YFP-editing-reporter (which co-expresses a red fluorescent transfection control), the N-terminal ABE8e vector, the C-terminal ABE8e vector, and a mdx locus targeting gRNA.

To demonstrate in vivo activity of the split ABE8e base editor that is reconstituted using efficient synthetic intron sequences, FIG. 23F shows in vivo editing of the mdx premature stop codon resulting in expression of dystrophin in treated muscle. A male mdx mutant mouse is injected with a mixture of N- and C-terminal ABE8e constructs packaged in adeno-associated virus 8 vectors. The virus genomes each contain two gRNA expression cassettes (composed of an RNA polymerase I promoter and the gRNA sequence). The virus mix was injected intra muscularly into the tibialis anterior muscle. Top right panel shows dystrophin staining in a wild-type tibialis anterior muscle cross section for reference (e.g., positive control). The bottom left panel shows untreated tibialis anterior muscle tissue (e.g., negative control). The bottom right panel shows expression of dystrophin in a tibialis anterior muscle that was injected with the two adeno-associated viruses.

To further demonstrate correction of the premature stop codon using the split ABE8e base editor that is reconstituted using efficient synthetic intron sequences, FIG. 23G shows dystrophin expression (top panel), ABESe expression (middle panel), and a GAPDH loading control (bottom panel) of tibialis anterior muscle treated with the adeno-associated virus mix for expression of ABE8e. This illustrates the rescue of dystrophin expression using the ABE8e base editor.

Based on these observations, expression of a full-length ABE8e protein in vivo can be achieved, for example to treat genomic point mutations. For example, a first RNA molecule comprises a first portion of the ABE8e protein coding sequence that is appended to a first synthetic RNA dimerization and recombination domain (that is an intron and binding domain). This molecule is expressed from a first vector/plasmid. A second RNA molecule comprises a second portion of the ABE8e protein coding sequence appended to the complementary second RNA dimerization and recombination domain and is expressed from a second vector/plasmid. One or both of these DNA vectors/plasmids contain an gRNA/guideRNA/gRNA expression cassette composed of an RNA polymerase III promoter and the gRNA/guideRNA/gRNA sequence as illustrated for example in FIG. 6G. For example, a composition of the present disclosure may include at least 1, at least 2, at least 3, at least 4, at least 5, at least 6, at least 7, at least 8 gRNA/guideRNA/gRNA expression cassettes. When the two RNA molecules are expressed in the same target cell, the two portions of ABE8e coding transcript are recombined to form the full-length ABE8e transcript which is then translated into protein. For example, a sequence having at least 80%, at least 90%, at least 95%, at least 96%, at least 97%, at least 98%, at least 99% or 100% sequence identity to SEQ ID NO: 165, which includes an N-terminal ABE8e coding sequence, and SEQ ID NO: 166 which includes a C-terminal ABE8e coding sequence, can be utilized for in vivo expression.

### Example 24

### Increasing RNA Fragment Length Results in Decreasing Two-Way Split Gene Reconstitution

The impact of length of the 5′ fragment encoding RNA molecule and the 3′ fragment encoding RNA molecule was assessed.

The yellow fluorescent protein (yfp) coding sequence was split into two fragments. To extend the RNA encoding molecules, stuffer open reading frames were installed at the 5′ end of the 5′ fragment and the 3′ end of the 3′ fragment, respectively. The 5′ yfp coding sequence was fused to an extended stuffer open reading frame via a self-cleaving 2A sequence. The 3′ yfp coding sequence of yfp was linked via a self-cleaving 2A sequence to an extended stuffer open reading frame. At the split point of the 5′ fragment of yfp and the 3′ fragment of yfp an RNA end joining module (synthetic intron plus binding domain) was installed. The self-cleaving 2A sequences allow for the YFP protein to separate from the respective stuffer open reading frames after translation. By incorporating different length stuffer open reading frames, four 5′ fragment encoding constructs and four 3′ fragment encoding constructs were assembled. The length of the RNA (protein coding sequence plus synthetic intron and binding domain) transcribed from these constructs were: 1000 nt, 2000 nt, 3000 nt, and 4000 nt for the 5′ fragment and 1000 nt, 2000 nt, 3000 nt, and 4000 nt for the 3′ fragment.

Efficiency of YFP reconstitution was compared between all sixteen 5′ to 3′ fragment pairs. In this comparison, YFP was most efficiently reconstituted when the shortest constructs (i.e., 5′-1000 nt with 3′-1000 nt) were paired. A decline of reconstitution efficiency was observed when fragments with longer stuffer sequences were paired. As percent of the shortest pairing (5′-1000 nt with 3′-1000 nt) the following YFP reconstitution efficiencies were observed:
5′-1000 nt with 3′-1000 nt: 100%
5′-1000 nt with 3′-2000 nt: ˜40%
5′-1000 nt with 3′-3000 nt: ˜20%
5′-1000 nt with 3′-4000 nt: ˜16%
5′-2000 nt with 3′-1000 nt: ˜55%
5′-2000 nt with 3′-2000 nt: ˜30%
5′-2000 nt with 3′-3000 nt: ˜20%
5′-2000 nt with 3′-4000 nt: ˜15%
5′-3000 nt with 3′-1000 nt: ˜60%
5′-3000 nt with 3′-2000 nt: ˜40%
5′-3000 nt with 3′-3000 nt: ˜25%
5′-3000 nt with 3′-4000 nt: ˜20%
5′-4000 nt with 3′-100 nt: ˜40%
5′-4000 nt with 3′-2000 nt: ˜35%
5′-4000 nt with 3′-3000 nt: ˜20%
5′-4000 nt with 3′-4000 nt: ˜15%.
   These data illustrate that increasing the length of the fragments that encode the 5′ and 3′ coding sequences of a split gene progressively lowers the efficiency of split gene reconstitution.

### Example 25

Enhancement of RNA End Joining Reaction with Downstream Intronic Splicing Enhancer and Intronic Splicing Enhancer Sequences

This example describes methods used to achieve efficient joining of two RNA molecules by incorporation of specific splicing enhancer sequences.

The effectiveness of select intronic splicing enhancer sequences was investigated using a screening platform where a split yellow fluorescent protein (YFP) was reconstituted using RNA end joining modules that were composed of a trimodal kissing loop RNA dimerization domain and a variable library of intronic segments. The sequences of the 5′ and 3′ DNA molecules used are shown in SEQ ID NOS: 171 and 172, respectively (the string of Ns in the sequence indicates the site of intronic library placement, such as at least one of the sequences in Table 5 below, such as 1, 2, 3, 4 or 5 of these sequences).

To demonstrate expression of reconstituted yfp in vitro, flow cytometry was used to determine yfp fluorescence intensity in HEK293t cells that were transfected with the 5′ and 3′ DNA molecules. As shown in FIG. 24A, the intronic portion of the disclosed split-REJ system was subdivided into individual segments to screen for efficient intronic splicing enhancer sequences that facilitate the RNA joining reaction. The sequences used in the three positions of the 5′ and the three positions of the 3′ intronic portion of the constructs are given in SEQ NOS: 173 to 204 (Table 5) and listed in FIG. 24C.

TABLE 5
Exemplary Intronic Splicing Enhancer Sequences

| label | name | Sequence (SEQ ID NO) |
|---|---|---|
| ds1 | FGFR-2 pre-mRNA, IAS1 | GTAAGTATTgctttcatttttgtctttttttaa (173) |
| ds2 | Fas URI6 | GTAagttcttgctttgttcaaactgtctat (174) |
| ds3 | CFTR E9 PY1/2 | GTAAGTATTCTTTTGTTCTTCACtcat (175) |
| ds4 | TIA1-preferred | GTAAGTATTTTTTTACTCCtcaTTTTTACTCC (176) |
| ds5 | FAS intron5 | GTAAGTATTTTTTTACGGTTATATTCTCCTTTCCCC (177) |
| ds6 | CD46-D1/9 | GTAAGTATTTTCTGTTGTTTATTttcag (178) |
| ds7 | B19V ISE | GTAAGTATTGGGGTTGATTATGTGTGGGACGGTGTAAGG |
| | | (179) |
| ds8 | ratFGFR2DISE | GTAAGTATTtcctctttctttccatgggttggcct (180) |
| ds9 | just donor scramble | GTAAGTATTaccagagattogtagacctgcttgac (181) |
| m1 | 6x WGGG | TGGGGCTGGGCAGAGGGTTGAGGGGAGAGGGTCCTGGGG |
| | | (182) |
| m2 | C9-E6-ISE | tcaTGGGTGGGTtcatTGGGTGGGTtca (183) |
| m3 | AdMLBPadj | Tagggcgcagtagtccagggttt (184) |
| m4 | bc12-12-BPadj | Ttctctgtggggggcattctctgctctct (185) |
| m5 | M2 | GGGttatGGGACCtcaGGGataaGGGACC (186) |
| m6 | GHlivs | CGGGGATGGGGGtca (187) |
| m7 | WangGrich | TGGGGGGAGGtcaTGGGGGGAGG (188) |
| m8 | WangISE2 | GTTGGTGGTTtcatGTTGGTGGTT (189) |
| m9 | WangA | GGGTTTCGGGTTTtcaGGTGGTCGTTGGT (190) |
| m10 | WangB | GGTGGTCGTTGGTtcaTTTGGGCTATTGG (191) |
| m11 | WangC | TTTGGGCTATTGGtcaAGGGGGCGAGGGG (192) |
| m12 | WangD | AGGGGGCGAGGGGtcaGGTATTCGGTATT (193) |
| m13 | WangE | GGTATTCGGTATTtcaaggtaaCaggtaa (194) |
| m14 | WangFmod | aggtaaCaggtaatcaGGGTTTCGGGTTT (195) |
| m15 | SMN-URC2/3 | TCTTACTTTTGTaaacTTTATGGTTTGTg (196) |
| m16 | just scramble | Cacgtattctcggtacggacgttacaga (197) |
| dd1 | scramble | Taagctggtatcc (198) |
| ap2 | 4.1R-E16-uISE | CACTAACTCTTTTTCCCCCCttttttttttACAG (199) |
| ap3 | P6-cons-to30 | TACTAACtctttcttttttCCTTTCCTTCTTCACAG (200) |
| ap4 | AdMLSA | CACTAACTCTgtcatacttatcctgtcccttttttttccaCAG (201) |
| ap5 | bc12-12-SA | CACTAACTCTctttctttttcttccctcctctcccccaactgCAG (202) |
| ap6 | perfectT | CACTAACTCTtttttttttttttttttttttACAGCAG (203) |
| ad1 | scramble | Taagctggtatcc (204) |

Quantification of the flow cytometry is shown in FIG. 24B. Incorporation of intronic sequences to stimulate recruitment of the 5′ splice site selection promoting splicing factor TIA-1 (T-Cell-Restricted Intracellular Antigen-1) can increase RNA end joining. In some examples, sequences containing WGGG motifs enhance RNA end joining.

Based on these observations, expression of a full-length split proteins in vivo can be enhanced by incorporating specific intronic splicing enhancer sequences into the intronic portion of the RNA end joining modules. For example, one or more sequences (such as 1, 2, or 3 sequences) having at least 80%, at least 90%, at least 95%, at least 96%, at least 97%, at least 98%, at least 99% or 100% sequence identity to any one of SEQ NOS: 173 to 180, 182-196, or 199 to 203, can be utilized for in vivo expression of RNA end joining reaction products (for example can be use as the ISE for any embodiment provided herein).

### Example 26

### Correction of Dystrophin In Vivo

This example describes methods and results further demonstrating that the disclosed methods can be used to edit a gene in vivo.

The two-part (dual) REJ Cas9-Adenine base editor (ABE) system used was that described in Example 23, comprising SEQ ID NOS: 225 and 226, each packaged in an AAV8 vector. In these two constructs (first and second DNA molecules) the nucleic acid editing protein is encoded in two parts by subsequences SEQ ID NOS: 165 and 166, respectively. The system was administered to tibialis anterior muscle of mdx-4cv mice (which have a mutant Dmd gene, and exhibit muscle fiber necrosis, fibrosis and centrally nucleated skeletable muscle fibers) by direct intramuscular injection of the two AAV8 vectors (total 2E11 viral genomes per muscle). Injection was done at 4 weeks of age followed by euthanasia at 8 weeks. Cas9-ABE and dystrophin expression in muscle extracts were quantified by western blot in wildtype (wt), untreated, and treated mice.

As shown in FIG. 25A, treated mdx-4cv mice have robust expression of Cas9-ABE, resulting in dystrophin expression restoration in muscle, while untreated mdx-4cv mice do not show such expression. GAPDH expression was used to monitor protein-extract concentration.

As shown in FIG. 25B, dystrophin expression in muscle visualized by immunohistochemistry using anti-dystrophin primary antibody demonstrates that treated mice show restoration of the characteristic dystrophin-sacroglycan complex in muscle sections while untreated mice do not.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

1. A composition for expressing a nucleic acid editing protein, comprising:
(a) a first RNA molecule comprising from 5′ to 3′:
(i) a coding sequence for an N-terminal portion of the nucleic acid editing protein;
(ii) a splice donor; and
(iii) a first dimerization domain; and
(b) a second RNA molecule comprising from 5′ to 3′:
(i) a second dimerization domain, wherein the second dimerization domain binds to the first dimerization domain;
(ii) a branch point sequence;
(iii) a polypyrimidine tract;
(iv) a splice acceptor; and
(v) a coding sequence for a C-terminal portion of the nucleic acid editing protein;
(c) optionally, a third RNA molecule comprising at least one first guide RNA (gRNA) specific for a first target nucleic acid molecule, wherein the at least one first gRNA directs the nucleic acid editing protein to a target editing site on the first target nucleic acid molecule;
(d) optionally, a fourth RNA molecule comprising at least one second gRNA specific for (i) the first target nucleic acid molecule, wherein the at least one second gRNA directs the nucleic acid editing protein to the same or different target editing site on the first nucleic acid molecule, or i(i) a second target nucleic acid molecule, wherein the at least one second gRNA directs the nucleic acid editing protein to a target editing site on the second target nucleic acid molecule
(e) optionally, a fifth RNA molecule comprising at least one third gRNA specific for (i) the first target nucleic acid molecule, wherein the at least one third gRNA directs the nucleic acid editing protein to the same or different target editing site on the first nucleic acid molecule as the first and second gRNA, (ii) the second target nucleic acid molecule, wherein the at least one third gRNA directs the nucleic acid editing protein to the same or different target editing site on the second nucleic acid molecule as the second gRNA, or (iii) a third target nucleic acid molecule, wherein the at least one third gRNA directs the nucleic acid editing protein to a target editing site on the third target nucleic acid molecule; and
(f) optionally, a sixth RNA molecule comprising at least one fourth gRNA specific for (i) the first target nucleic acid molecule, wherein the at least one fourth gRNA directs the nucleic acid editing protein to the same or different target editing site on the first nucleic acid molecule as the first, second, and third gRNA, (ii) the second target nucleic acid molecule, wherein the at least one fourth gRNA directs the nucleic acid editing protein to the same or different target editing site on the second nucleic acid molecule as the second and third gRNA, (iii) the third target nucleic acid molecule, wherein the at least one fourth gRNA directs the nucleic acid editing protein to the same or different target editing site on the third target nucleic acid molecule as the third gRNA or (iv) a fourth target nucleic acid molecule, wherein the at least one fourth gRNA directs the nucleic acid editing protein to a target editing site on the fourth target nucleic acid molecule.

2. The composition of claim 1, wherein the first and second dimerization domains bind by direct binding, indirect binding, or a combination thereof.

3. The composition of claim 2, wherein direct binding or indirect binding comprises basepairing interactions, non-canonical base pairing interactions, non-base pairing interactions, or a combination thereof.

4. The composition of claim 2, wherein
direct binding comprises base pairing interactions between kissing loops or hypodiverse regions,
direct binding comprises non-canonical basepairing interactions, non-canonical base pairing interactions, non-base pairing interactions, or a combination thereof, between aptamer regions:
indirect binding comprises basepairing interactions through a nucleic acid bridge, and/or indirect binding comprises non-base pairing interactions between an aptamer and an aptamer target, or between two aptamers.

5.-8. (canceled)

9. The composition of claim 1, wherein the dimerization domains are directly binding or indirectly binding aptamer sequence dimerization domains.

10. The composition of claim 1, wherein the dimerization domains are kissing loop interaction domains.

11. The composition of claim 1, wherein the target editing site is part of a target nucleic acid molecule or a regulatory region of a target nucleic acid molecule associated with disease.

12.-13. (canceled)

14. The composition ofany one of claim 11, wherein the disease and the first, second, third, and/or fourth target nucleic acid molecule are one listed in Tables 1-4.

15. The composition of claim 1, wherein the first RNA molecule further comprises one or both of a downstream intronic splice enhancer (DISE) 3′ to the splice donor and 5′ to the first dimerization domain, an intronic splice enhancer (ISE) 3′ to the splice donor and 5′ to the first dimerization domain; and/or
the second RNA molecule further comprises one or both of an ISE 3′ to the second dimerization domain and 5′ to the branch point sequence, and a DISE 3′ to the splice donor and 5′ to the dimerization domain;
or any combination thereof.

16. The composition of claim 1, wherein
the first RNA molecule further comprises a self-cleaving RNA sequence or an RNA-cleaving enzyme target sequence positioned anywhere 3′ to the splice donor such that it cleaves off the 3′ located polyadenylated tail to decrease or suppress protein fragment expression from a non-recombined RNA molecule;
the second RNA molecule further comprises a self-cleaving RNA sequence or an RNA-cleaving enzyme target sequence positioned anywhere 5′ to the branch point sequence such that it cleaves off the 5′ located RNA cap to decrease or suppress protein fragment expression from a non-recombined RNA molecule;
the second RNA molecule further comprises a start codon anywhere 5′ to the branch point sequence that is shifted relative to the open reading frame 3′ of the splice acceptor to decrease or suppress translation of a nucleic acid editing protein fragment from a non-recombined RNA molecule;
the first RNA molecule further comprises a micro RNA target site anywhere 3′ to the splice donor such that an un-joined RNA fragment undergoes micro RNA dependent degradation once outside the nucleus;
the second RNA molecule further comprises a micro RNA target site anywhere 3′ to the coding sequence such that an un-joined RNA fragment undergoes micro RNA dependent degradation once outside the nucleus;
the first RNA molecule further comprises a sequence encoding a degron protein degradation tag anywhere 3′ to the splice donor such that it is in frame with the nucleic acid editing protein open reading frame 5′ of the splice donor site such that an un-joined protein fragment is tagged for degradation;
the second RNA molecule further comprises a start codon and an in-frame degron protein degradation tag anywhere 5′ to the branch point sequence such that it is in frame with the nucleic acid editing protein open reading frame 3′ of the splice acceptor site such that an un-joined protein fragment is tagged for degradation;
or any combination thereof.

17. The composition of claim 1, wherein the nucleic acid editing protein comprises a Cas nuclease, zinc finger nuclease, or transcription activator-like effector nuclease.

18. The composition of claim 1, wherein
the first, second, third, and/or fourth target nucleic acid molecule are target DNA molecules, and the at least one first, second, third, and/or fourth gRNA comprises a crRNA and tracrRNA, or
the first, second, third, and/or fourth target nucleic acid molecule are target RNA, and the at least one first, second, third, and/or fourth gRNA comprises one or more direct repeats and one or more spacers.

19. The composition of claim 18, wherein the nucleic acid editing protein comprises Cas9, Cas13a, Cas13b, Cas13c, Cas13d, dead Cas13d (dCas13d), or dead Cas9 (dCas9).

20. (canceled)

21. The composition of claim 19, wherein the Cas9, Cas13a, Cas13b, Cas13c, Cas13d, dCas13d or dCas9 is part of a fusion protein.

22.-27. (canceled)

28. The composition of claim 1, wherein the at least one first, second, third, and/or fourth gRNA comprise multiple copies of each of the first, second, third, and/or fourth gRNA.

29. The composition of claim 1, further comprising a parvovirus inverted terminal repeat (ITR) at the 5′ and the 3′-end of each of the first RNA molecule and the second RNA molecule.

30. A DNA molecule composition for expressing the composition of claim 1, comprising:
(i) a first synthetic DNA molecule encoding the RNA molecules of (a), and optionally (c), (d), or both (c) and (d); and
(ii) a second synthetic DNA molecule encoding the RNA molecule of (b), and optionally and (e), (f), or both (e) and (f).

31. The DNA molecule of claim 30, further comprising:
(a) a first promoter operably linked to a DNA sequence encoding the N-terminal portion of the nucleic acid editing protein;
(b) a second promoter operably linked to a DNA sequence encoding the C-terminal portion of the nucleic acid editing protein
(c) if the first synthetic DNA molecule includes a DNA molecule encoding the RNA molecule of (c), a third promoter operably linked to a sequence encoding the at least one first gRNA;
(d) if the first synthetic DNA molecule includes a DNA molecule encoding the RNA molecule of (d), a fourth promoter operably linked to a sequence encoding the at least one second gRNA;
(e) if the second synthetic DNA molecule includes a DNA molecule encoding the RNA molecule of (e), a fifth promoter operably linked to a sequence encoding the at least one third gRNA; and
(f) if the second synthetic DNA molecule includes a DNA molecule encoding the RNA molecule of (d), a sixth promoter operably linked to a sequence encoding the at least one fourth gRNA

32. (canceled)

33. The composition of claim 31, wherein:
the first and second promoter are the same promoter;
the first and second promoter are different promoters,
the third, fourth, fifth and sixth promoters are the same promoter;
the third, fourth, fifth and sixth promoters are different promoters, or
combinations thereof.

34. The composition of claim 30, wherein each of the first and second promoters is independently selected from: a constitutive promoter; a tissue-specific promoter; and a promoter endogenous to the nucleic acid editing protein.

35. (canceled)

36. A system for expressing a nucleic acid editing protein comprising a composition of claim 30.

37. The system of claim 36, wherein when the system is introduced into a cell the RNA molecules are produced and recombine in the proper order, resulting in a full-length coding sequence of the nucleic acid editing protein.

38. The system of claim 36, wherein each of the synthetic first and second RNA molecules are transcribed from a separate viral vector.

39. (canceled)

40. The system of any claim 36, wherein
each of the synthetic DNA molecules has a size independently selected from: about 2500 nt to about 5000 nt, 2,500 nt to about 2,750 nt, about 2,500 nt to about 3,000 nt, about 2,500 nt to about 3,250 nt, about 2,500 nt to about 3,500 nt, about 2,500 nt to about 3,750 nt, about 2,500 nt to about 4,000 nt, about 2,500 nt to about 4,250 nt, about 2,500 nt to about 4,500 nt, about 2,500 nt to about 4,750 nt, about 2,500 nt to about 5,000 nt, about 2,750 nt to about 3,000 nt, about 2,750 nt to about 3,250 nt, about 2,750 nt to about 3,500 nt, about 2,750 nt to about 3,750 nt, about 2,750 nt to about 4,000 nt, about 2,750 nt to about 4,250 nt, about 2,750 nt to about 4,500 nt, about 2,750 nt to about 4,750 nt, about 2,750 nt to about 5,000 nt, about 3,000 nt to about 3,250 nt, about 3,000 nt to about 3,500 nt, about 3,000 nt to about 3,750 nt, about 3,000 nt to about 4,000 nt, about 3,000 nt to about 4,250 nt, about 3,000 nt to about 4,500 nt, about 3,000 nt to about 4,750 nt, about 3,000 nt to about 5,000 nt, about 3,250 nt to about 3,500 nt, about 3,250 nt to about 3,750 nt, about 3,250 nt to about 4,000 nt, about 3,250 nt to about 4,250 nt, about 3,250 nt to about 4,500 nt, about 3,250 nt to about 4,750 nt, about 3,250 nt to about 5,000 nt, about 3,500 nt to about 3,750 nt, about 3,500 nt to about 4,000 nt, about 3,500 nt to about 4,250 nt, about 3,500 nt to about 4,500 nt, about 3,500 nt to about 4,750 nt, about 3,500 nt to about 5,000 nt, about 3,750 nt to about 4,000 nt, about 3,750 nt to about 4,250 nt, about 3,750 nt to about 4,500 nt, about 3,750 nt to about 4,750 nt, about 3,750 nt to about 5,000 nt, about 4,000 nt to about 4,250 nt, about 4,000 nt to about 4,500 nt, about 4,000 nt to about 4,750 nt, about 4,000 nt to about 5,000 nt, about 4,250 nt to about 4,500 nt, about 4,250 nt to about 4,750 nt, about 4,250 nt to about 5,000 nt, about 4,500 nt to about 4,750 nt, about 4,500 nt to about 5,000 nt, about 4,750 nt to about 5,000 nt, about 2,500 nt, about 2,750 nt, about 3,000 nt, about 3,250 nt, about 3,500 nt, about 3,750 nt, about 4,000 nt, about 4,250 nt, about 4,500 nt, about 4,750 nt, and about 5,000 nt,
the coding sequence for an N-terminal portion of the nucleic acid editing protein, or a C-terminal portion of the nucleic acid editing protein encoded by a synthetic DNA molecule of the system each has a size independently selected from: about 2500 to 4500 nt, about 2,500 nt to about 2,750 nt, about 2,500 nt to about 3,000 nt, about 2,500 nt to about 3,250 nt, about 2,500 nt to about 3,500 nt, about 2,500 nt to about 3,750 nt, about 2,500 nt to about 4,000 nt, about 2,500 nt to about 4,250 nt, about 2,500 nt to about 4,500 nt, about 2,750 nt to about 3,000 nt, about 2,750 nt to about 3,250 nt, about 2,750 nt to about 3,500 nt, about 2,750 nt to about 3,750 nt, about 2,750 nt to about 4,000 nt, about 2,750 nt to about 4,250 nt, about 2,750 nt to about 4,500 nt, about 3,000 nt to about 3,250 nt, about 3,000 nt to about 3,500 nt, about 3,000 nt to about 3,750 nt, about 3,000 nt to about 4,000 nt, about 3,000 nt to about 4,250 nt, about 3,000 nt to about 4,500 nt, about 3,250 nt to about 3,500 nt, about 3,250 nt to about 3,750 nt, about 3,250 nt to about 4,000 nt, about 3,250 nt to about 4,250 nt, about 3,250 nt to about 4,500 nt, about 3,500 nt to about 3,750 nt, about 3,500 nt to about 4,000 nt, about 3,500 nt to about 4,250 nt, about 3,500 nt to about 4,500 nt, about 3,750 nt to about 4,000 nt, about 3,750 nt to about 4,250 nt, about 3,750 nt to about 4,500 nt, about 4,000 nt to about 4,250 nt, about 4,000 nt to about 4,500 nt, about 4,250 nt to about 4,500 nt, about 2,500 nt, about 2,750 nt, about 3,000 nt, about 3,250 nt, about 3,500 nt, about 3,750 nt, about 4,000 nt, about 4,250 nt, and about 4,500 nt:
any one or both of the RNA molecules encoded by the synthetic DNA molecules of the system, respectively, has a size independently selected from: about 2500 to 4500 nt, about 2,500 nt to about 2,750 nt, about 2,500 nt to about 3,000 nt, about 2,500 nt to about 3,250 nt, about 2,500 nt to about 3,500 nt, about 2,500 nt to about 3,750 nt, about 2,500 nt to about 4,000 nt, about 2,500 nt to about 4,250 nt, about 2,500 nt to about 4,500 nt, about 2,750 nt to about 3,000 nt, about 2,750 nt to about 3,250 nt, about 2,750 nt to about 3,500 nt, about 2,750 nt to about 3,750 nt, about 2,750 nt to about 4,000 nt, about 2,750 nt to about 4,250 nt, about 2,750 nt to about 4,500 nt, about 3,000 nt to about 3,250 nt, about 3,000 nt to about 3,500 nt, about 3,000 nt to about 3,750 nt, about 3,000 nt to about 4,000 nt, about 3,000 nt to about 4,250 nt, about 3,000 nt to about 4,500 nt, about 3,250 nt to about 3,500 nt, about 3,250 nt to about 3,750 nt, about 3,250 nt to about 4,000 nt, about 3,250 nt to about 4,250 nt, about 3,250 nt to about 4,500 nt, about 3,500 nt to about 3,750 nt, about 3,500 nt to about 4,000 nt, about 3,500 nt to about 4,250 nt, about 3,500 nt to about 4,500 nt, about 3,750 nt to about 4,000 nt, about 3,750 nt to about 4,250 nt, about 3,750 nt to about 4,500 nt, about 4,000 nt to about 4,250 nt, about 4,000 nt to about 4,500 nt, about 4,250 nt to about 4,500 nt, about 2,500 nt, about 2,750 nt, about 3,000 nt, about 3,250 nt, about 3,500 nt, about 3,750 nt, about 4,000 nt, about 4,250 nt, and about 4,500 nt:
the first dimerization domain and the second dimerization domain are each no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150, 200, 250, 300, 400, or 500 nt; and the system has a recombination efficiency of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%:
each dimerization domain is no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150, 200, 250, 300, 400, or 500 nt; and the system has a recombination efficiency of at least 20%, at least 30%, at least 40%, at least 50%, at least 60%, at least 70%, at least 75%, at least 80%, at least 90%, or about 100%; and/or
the RNA recombination efficiency is about 10% to about 100%, about 10% to about 20%, about 10% to about 30%, about 10% to about 35%, about 10% to about 40%, about 10% to about 45%, about 10% to about 50%, about 10% to about 55%, about 10% to about 60%, about 10% to about 70%, about 10% to about 80%, about 10% to about 90%, about 20% to about 30%, about 20% to about 35%, about 20% to about 40%, about 20% to about 45%, about 20% to about 50%, about 20% to about 55%, about 20% to about 60%, about 20% to about 70%, about 20% to about 80%, about 20% to about 90%, about 30% to about 35%, about 30% to about 40%, about 30% to about 45%, about 30% to about 50%, about 30% to about 55%, about 30% to about 60%, about 30% to about 70%, about 30% to about 80%, about 30% to about 90%, about 35% to about 40%, about 35% to about 45%, about 35% to about 50%, about 35% to about 55%, about 35% to about 60%, about 35% to about 70%, about 35% to about 80%, about 35% to about 90%, about 40% to about 45%, about 40% to about 50%, about 40% to about 55%, about 40% to about 60%, about 40% to about 70%, about 40% to about 80%, about 40% to about 90%, about 45% to about 50%, about 45% to about 55%, about 45% to about 60%, about 45% to about 70%, about 45% to about 80%, about 45% to about 90%, about 50% to about 55%, about 50% to about 60%, about 50% to about 70%, about 50% to about 80%, about 50% to about 90%, about 55% to about 60%, about 55% to about 70%, about 55% to about 80%, about 55% to about 90%, about 60% to about 70%, about 60% to about 80%, about 60% to about 90%, about 70% to about 80%, about 70% to about 90%, about 80% to about 90%, about 10%, about 20%, about 30%, about 35%, about 40%, about 45%, about 50%, about 55%, about 60%, about 70%, about 80%, about 90%, about 95%, or about 100%.

41.-46. (canceled)

47. A composition comprising the system of claim 36.

48. The composition of claim 47, wherein the composition comprises first, second, third and optionally fourth RNA molecules, each encoding at least a portion of a nucleic acid editing protein.

49. (canceled)

50. A method of expressing a nucleic acid editing protein in a cell, comprising:
introducing the system of claim 36 into a cell, and expressing the first and second RNA molecules in the cell, wherein the nucleic acid editing protein is produced in the cell.

51. The method of claim 50, wherein the cell is in a subject, and introducing comprises administering a therapeutically effective amount the system to the subject.

52. The method of claim 51, wherein the method treats a genetic disease caused by a mutation in a target nucleic acid molecule in the subject, wherein the method results in expression of the nucleic acid editing protein and optionally the first and second gRNAs and correction of the mutation in the subject.

53. (canceled)

54. The system of claim 36, wherein
one or both of the first and second RNA molecules comprise at least 80%, at least 85%, at least 90%, at least 95%, at least 98%, at least 99%, or 100% sequence identity to a synthetic intron provided in any one of SEQ ID NOS: 159, 160, 161, 162, 163, 164, 165, 166, 225, and 226;
one or both of the first and second RNA molecules comprise a synthetic intron selected from the RNA encoded by SEO ID NO: 159, 160, 161, 162, 163, 164, 165, 166, 225, or 22:
one or both of the first and second RNA molecules further comprise a portion of a protein coding sequence;
the portion of the protein coding sequence comprises an N-terminal half, an N-terminal portion, a C-terminal half, or a C-terminal portion, of the protein coding sequence.

55.-58. (canceled)

59. The composition of claim 1, wherein
any one or two of the first and second RNA molecules each has a size independently selected from: about 2500 to 4500 nt, about 2,500 nt to about 2,750 nt, about 2,500 nt to about 3,000 nt, about 2,500 nt to about 3,250 nt, about 2,500 nt to about 3,500 nt, about 2,500 nt to about 3,750 nt, about 2,500 nt to about 4,000 nt, about 2,500 nt to about 4,250 nt, about 2,500 nt to about 4,500 nt, about 2,750 nt to about 3,000 nt, about 2,750 nt to about 3,250 nt, about 2,750 nt to about 3,500 nt, about 2,750 nt to about 3,750 nt, about 2,750 nt to about 4,000 nt, about 2,750 nt to about 4,250 nt, about 2,750 nt to about 4,500 nt, about 3,000 nt to about 3,250 nt, about 3,000 nt to about 3,500 nt, about 3,000 nt to about 3,750 nt, about 3,000 nt to about 4,000 nt, about 3,000 nt to about 4,250 nt, about 3,000 nt to about 4,500 nt, about 3,250 nt to about 3,500 nt, about 3,250 nt to about 3,750 nt, about 3,250 nt to about 4,000 nt, about 3,250 nt to about 4,250 nt, about 3,250 nt to about 4,500 nt, about 3,500 nt to about 3,750 nt, about 3,500 nt to about 4,000 nt, about 3,500 nt to about 4,250 nt, about 3,500 nt to about 4,500 nt, about 3,750 nt to about 4,000 nt, about 3,750 nt to about 4,250 nt, about 3,750 nt to about 4,500 nt, about 4,000 nt to about 4,250 nt, about 4,000 nt to about 4,500 nt, about 4,250 nt to about 4,500 nt, about 2,500 nt, about 2,750 nt, about 3,000 nt, about 3,250 nt, about 3,500 nt, about 3,750 nt, about 4,000 nt, about 4,250 nt, and about 4,500 nt;
the total nucleic acid editing protein coding sequence size is about 2000 nt to about 8000 nt, about 2,000 nt to about 3,000 nt, about 2,000 nt to about 3,500 nt, about 2,000 nt to about 4,000 nt, about 2,000 nt to about 4,500 nt, about 2,000 nt to about 5,000 nt, about 2,000 nt to about 5,500 nt, about 2,000 nt to about 6,000 nt, about 2,000 nt to about 6,500 nt, about 2,000 nt to about 7,000 nt, about 2,000 nt to about 7,500 nt, about 2,000 nt to about 8,000 nt, about 3,000 nt to about 3,500 nt, about 3,000 nt to about 4,000 nt, about 3,000 nt to about 4,500 nt, about 3,000 nt to about 5,000 nt, about 3,000 nt to about 5,500 nt, about 3,000 nt to about 6,000 nt, about 3,000 nt to about 6,500 nt, about 3,000 nt to about 7,000 nt, about 3,000 nt to about 7,500 nt, about 3,000 nt to about 8,000 nt, about 3,500 nt to about 4,000 nt, about 3,500 nt to about 4,500 nt, about 3,500 nt to about 5,000 nt, about 3,500 nt to about 5,500 nt, about 3,500 nt to about 6,000 nt, about 3,500 nt to about 6,500 nt, about 3,500 nt to about 7,000 nt, about 3,500 nt to about 7,500 nt, about 3,500 nt to about 8,000 nt, about 4,000 nt to about 4,500 nt, about 4,000 nt to about 5,000 nt, about 4,000 nt to about 5,500 nt, about 4,000 nt to about 6,000 nt, about 4,000 nt to about 6,500 nt, about 4,000 nt to about 7,000 nt, about 4,000 nt to about 7,500 nt, about 4,000 nt to about 8,000 nt, about 4,500 nt to about 5,000 nt, about 4,500 nt to about 5,500 nt, about 4,500 nt to about 6,000 nt, about 4,500 nt to about 6,500 nt, about 4,500 nt to about 7,000 nt, about 4,500 nt to about 7,500 nt, about 4,500 nt to about 8,000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 6,000 nt to about 6,500 nt, about 6,000 nt to about 7,000 nt, about 6,000 nt to about 7,500 nt, about 6,000 nt to about 8,000 nt, about 6,500 nt to about 7,000 nt, about 6,500 nt to about 7,500 nt, about 6,500 nt to about 8,000 nt, about 7,000 nt to about 7,500 nt, about 7,000 nt to about 8,000 nt, about 7,500 nt to about 8,000 nt, about 2,000 nt, about 3,000 nt, about 3,500 nt, about 4,000 nt, about 4,500 nt, about 5,000 nt, about 5,500 nt, about 6,000 nt, about 6,500 nt, about 7,000 nt, about 7,500 nt, or about 8,000 nt:
the summed size of the two RNA molecules is about 5,000 nt to about 9000 nt, about 5,000 nt to about 5,500 nt, about 5,000 nt to about 6,000 nt, about 5,000 nt to about 6,500 nt, about 5,000 nt to about 7,000 nt, about 5,000 nt to about 7,500 nt, about 5,000 nt to about 8,000 nt, about 5,000 nt to about 8,500 nt, about 5,000 nt to about 9,000 nt, about 5,500 nt to about 6,000 nt, about 5,500 nt to about 6,500 nt, about 5,500 nt to about 7,000 nt, about 5,500 nt to about 7,500 nt, about 5,500 nt to about 8,000 nt, about 5,500 nt to about 8,500 nt, about 5,500 nt to about 9,000 nt, about 6,000 nt to about 6,500 nt, about 6,000 nt to about 7,000 nt, about 6,000 nt to about 7,500 nt, about 6,000 nt to about 8,000 nt, about 6,000 nt to about 8,500 nt, about 6,000 nt to about 9,000 nt, about 6,500 nt to about 7,000 nt, about 6,500 nt to about 7,500 nt, about 6,500 nt to about 8,000 nt, about 6,500 nt to about 8,500 nt, about 6,500 nt to about 9,000 nt, about 7,000 nt to about 7,500 nt, about 7,000 nt to about 8,000 nt, about 7,000 nt to about 8,500 nt, about 7,000 nt to about 9,000 nt, about 7,500 nt to about 8,000 nt, about 7,500 nt to about 8,500 nt, about 7,500 nt to about 9,000 nt, about 8,000 nt to about 8,500 nt, about 8,000 nt to about 9,000 nt, about 8,500 nt to about 9,000 nt, about 5,000 nt, about 5,500 nt, about 6,000 nt, about 6,500 nt, about 7,000 nt, about 7,500 nt, about 8,000 nt, about 8,500 nt, or about 9,000 nt;
the first dimerization domain and the second dimerization domain are each no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150, 200, 250, 300, 400, or 500 nt; and the system has a recombination efficiency of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%:
each dimerization domain is no more than 1000 nt, such as at least 50 nt, at least 100 nt, at least 150 nt, at least 200 nt, at least 300 nt, at least 400 nt, at least 500 nt, 50 to 1000 nt, 50 to 500 nt, 50 to 150 nt, 50, 100, 150, 200, 250, 300, 400, or 500 nt; and the system has a recombination efficiency of at least 20%, at least 30%, at least 40%, at least 50%, at least 60%, at least 70%, at least 75%, at least 80%, or at least 90%; and/or
the RNA recombination efficiency is about 10% to about 100%, about 10% to about 20%, about 10% to about 30%, about 10% to about 35%, about 10% to about 40%, about 10% to about 45%, about 10% to about 50%, about 10% to about 55%, about 10% to about 60%, about 10% to about 70%, about 10% to about 80%, about 10% to about 90%, about 20% to about 30%, about 20% to about 35%, about 20% to about 40%, about 20% to about 45%, about 20% to about 50%, about 20% to about 55%, about 20% to about 60%, about 20% to about 70%, about 20% to about 80%, about 20% to about 90%, about 30% to about 35%, about 30% to about 40%, about 30% to about 45%, about 30% to about 50%, about 30% to about 55%, about 30% to about 60%, about 30% to about 70%, about 30% to about 80%, about 30% to about 90%, about 35% to about 40%, about 35% to about 45%, about 35% to about 50%, about 35% to about 55%, about 35% to about 60%, about 35% to about 70%, about 35% to about 80%, about 35% to about 90%, about 40% to about 45%, about 40% to about 50%, about 40% to about 55%, about 40% to about 60%, about 40% to about 70%, about 40% to about 80%, about 40% to about 90%, about 45% to about 50%, about 45% to about 55%, about 45% to about 60%, about 45% to about 70%, about 45% to about 80%, about 45% to about 90%, about 50% to about 55%, about 50% to about 60%, about 50% to about 70%, about 50% to about 80%, about 50% to about 90%, about 55% to about 60%, about 55% to about 70%, about 55% to about 80%, about 55% to about 90%, about 60% to about 70%, about 60% to about 80%, about 60% to about 90%, about 70% to about 80%, about 70% to about 90%, about 80% to about 90%, about 10%, about 20%, about 30%, about 35%, about 40%, about 45%, about 50%, about 55%, about 60%, about 70%, about 80%, about 90%, about 95%, or about 100%.

60.-63. (canceled)

64. The composition of claim 1, wherein:
(a) the first and second RNA molecules are each about 2500 nt to 4500 nt;
(b) the total nucleic acid editing protein coding sequence size is about 2000 nt to about 8000 nt; and/or
(c) the summed size of the two RNA molecules is about 5,000 nt to about 9000 nt;
and the RNA recombination efficiency is about 10% to about 100%.

65. The composition of claim 1, further comprising one or more additional RNA molecules comprising one or more gRNAs specific for one or more target nucleic acid molecules.

66. The composition of claim 30, further comprising one or more additional DNA molecules encoding one or more gRNAs specific for one or more target nucleic acid molecules.
